(19) United States
(12) Patent Application Publication
(10) Pub. No.: US 2005/0053930 A1
(43) Pub. Date: Mar. 10, 2005

(54) Therapeutic polypeptides, nucleic acids encoding same, and methods of use

(76) Inventors: David Anderson, Plantsville, CT (US); Ferenc Boldog, North Haven, CT (US); Catherine Burgess, Wethersfield, CT (US); Stacie Casman, North Haven, CT (US); Shlomit Edinger, New Haven, CT (US); Andrew Eisen, Rockville, MD (US); Karen Ellerman, Branford, CT (US); Valerie Gerlach, Branford, CT (US); Linda Gorman, Branford, CT (US); Xiaojia (Sasha) Guo, Branford, CT (US); Vladimir Gusev, Madison, CT (US); Weizhen Ji, Branford, CT (US); Li Li, Branford, CT (US); John MacDougall, Hamden, CT (US); Uriel Malyankar, North Branford, CT (US); Isabelle Millet, Milford, CT (US); Tatiana Ort, Milford, CT (US); Muralidhara Padigaru, Branford, CT (US); Sudhirdas K. Prayaga, O'Fallon, MO (US); Meera Patturajan, Branford, CT (US); Carol Pena, New Haven, CT (US); John Peyman, New Haven, CT (US); Daniel Rieger, Branford, CT (US); Mark Rothenberg, Clinton, CT (US); Paul Sciore, North Haven, CT (US); Suresh Shenoy, Branford, CT (US); Glennda Smithson, Guilford, CT (US); Kimberly Spytek, Ellington, CT (US); David Stone, Guilford, CT (US); Raymond Taupier JR., East Haven, CT (US); Velizar Tchernev, Branford, CT (US); Corine A.M. Vernet, North Branford, CT (US); Edward Voss, Wallingford, CT (US); Bryan Zerhusen, Branford, CT (US); Mei Zhong, Branford, CT (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/454,246

(22) Filed: Jun. 3, 2003

Related U.S. Application Data
(63) Continuation-in-part of application No. 09/898,994, filed on Jul. 3, 2001, now abandoned.
(60) Provisional application No. 60/218,903, filed on Jul. 18, 2000.
(60) Provisional application No. 60/387,002, filed on Jun. 7, 2002.
(60) Provisional application No. 60/385,504, filed on Jun. 4, 2002.
(60) Provisional application No. 60/386,974, filed on Jun. 6, 2002.
(60) Provisional application No. 60/386,453, filed on Jun. 6, 2002.
(60) Provisional application No. 60/386,041, filed on Jun. 5, 2002.
(60) Provisional application No. 60/386,816, filed on Jun. 7, 2002.
(60) Provisional application No. 60/387,540, filed on Jun. 10, 2002.
(60) Provisional application No. 60/403,486, filed on Aug. 13, 2002.
(60) Provisional application No. 60/387,659, filed on Jun. 11, 2002.
(60) Provisional application No. 60/403,522, filed on Aug. 14, 2002.
(60) Provisional application No. 60/396,706, filed on Jul. 17, 2002.
(60) Provisional application No. 60/389,742, filed on Jun. 17, 2002.
(60) Provisional application No. 60/387,037, filed on Jun. 7, 2002.
(60) Provisional application No. 60/402,832, filed on Aug. 12, 2002.
(60) Provisional application No. 60/389,123, filed on Jun. 13, 2002.
(60) Provisional application No. 60/403,748, filed on Aug. 15, 2002.
(60) Provisional application No. 60/389,729, filed on Jun. 17, 2002.
(60) Provisional application No. 60/390,006, filed on Jun. 19, 2002.
(60) Provisional application No. 60/387,934, filed on Jun. 12, 2002.

(51) Int. Cl. C12Q 1/68; C07K 14/47; A61K 38/17; C07H 21/04

(52) U.S. Cl. 435/6; 435/69.1; 435/320.1; 435/325; 530/350; 514/12; 536/23.5

(57) ABSTRACT

Disclosed herein are nucleic acid sequences that encode novel polypeptides. Also disclosed are polypeptides encoded by these nucleic acid sequences, and antibodies that immunospecifically bind to the polypeptide, as well as derivatives, variants, mutants, or fragments of the novel polypeptide, polynucleotide, or antibody specific to the polypeptide. The invention further discloses therapeutic, diagnostic and research methods for diagnosis, treatment, and prevention of disorders involving any one of these novel human nucleic acids and proteins.

### RELATED APPLICATIONS

This application is a continuation in part of U.S. Ser. No. 09/898,994, filed Jul. 3, 2001, which claims priority to U.S. Ser. No. 60/218,903, filed Jul. 18, 2000; U.S. Ser. No. 10/016,248, filed Dec. 10, 2001, which claims priority to U.S. Ser. No. 60/255,648, filed Dec. 14, 2000; U.S. Ser. No. 10/028,248, filed Dec. 19, 2001, which claims priority to U.S. Ser. No. 60/256,619, filed Dec. 19, 2000; U.S. Ser. No. 10/044,564, filed Jan. 11, 2002, which claims priority to U.S. Ser. No. 60/261,013, filed Jan. 11, 2001; U.S. Ser. No. 10/136,071, filed May 1, 2002, which claims priority to U.S. Ser. No. 60/289,087, filed May 7, 2001; and U.S. Ser. No. 09/908,193, filed Jul. 18, 2001; and this application claims priority to the following provisional applications: U.S. Ser. No. 60/387,002, filed Jun. 7, 2002; U.S. Ser. No. 60/385,504, filed Jun. 4, 2002; U.S. Ser. No. 60/386,974, filed Jun. 6, 2002; U.S. Ser. No. 60/386,453, filed Jun. 6, 2002; U.S. Ser. No. 60/386,041, filed Jun. 5, 2002; U.S. Ser. No. 60/386,816, filed Jun. 7, 2002; U.S. Ser. No. 60/387,540, filed Jun. 10, 2002; U.S. Ser. No. 60/403,486, filed Aug. 13, 2002; U.S. Ser. No. 60/365,491, filed Jun. 14, 2002; U.S. Ser. No. 60/387,659, filed Jun. 11, 2002; U.S. Ser. No. 60/403,522, filed Aug. 14, 2002; U.S. Ser. No. 60/387,934, filed Jun. 12, 2002; U.S. Ser. No. 60/390,006, filed Jun. 19, 2002; U.S. Ser. No. 60/389,729, filed Jun. 17, 2002; U.S. Ser. No. 60/403,748, filed Aug. 15, 2002; U.S. Ser. No. 60/389,123, filed Jun. 13, 2002; U.S. Ser. No. 60/402,832, filed Aug. 12, 2002; U.S. Ser. No. 60/387,037, filed Nov. 6, 2002; U.S. Ser. No. 60/389,742, filed Jun. 17, 2002; and U.S. Ser. No. 60/396,706, filed Jul. 17, 2002.

### FIELD OF THE INVENTION

The present invention relates to novel polypeptides, and the nucleic acids encoding them, having properties related to stimulation of biochemical or physiological responses in a cell, a tissue, an organ or an organism. More particularly, the novel polypeptides are gene products of novel genes, or are specified biologically active fragments or derivatives thereof. Methods of use encompass diagnostic and prognostic assay procedures as well as methods of treating diverse pathological conditions.

### BACKGROUND OF THE INVENTION

Eukaryotic cells are characterized by biochemical and physiological processes, which under normal conditions are exquisitely balanced to achieve the preservation and propagation of the cells. When such cells are components of multicellular organisms such as vertebrates or, more particularly, organisms such as mammals, the regulation of the biochemical and physiological processes involves intricate signaling pathways. Frequently, such signaling pathways involve extracellular signaling proteins, cellular receptors that bind the signaling proteins and signal transducing components located within the cells.

Signaling proteins may be classified as endocrine effectors, paracrine effectors or autocrine effectors. Endocrine effectors are signaling molecules secreted by a given organ into the circulatory system, which are then transported to a distant target organ or tissue. The target cells include the receptors for the endocrine effector, and when the endocrine effector binds, a signaling cascade is induced. Paracrine effectors involve secreting cells and receptor cells in close proximity to each other, for example, two different classes of cells in the same tissue or organ. One class of cells secretes the paracrine effector, which then reaches the second class of cells, for example by diffusion through the extracellular fluid. The second class of cells contains the receptors for the paracrine effector; binding of the effector results in induction of the signaling cascade that elicits the corresponding biochemical or physiological effect. Autocrine effectors are highly analogous to paracrine effectors, except that the same cell type that secretes the autocrine effector also contains the receptor. Thus the autocrine effector binds to receptors on the same cell, or on identical neighboring cells. The binding process then elicits the characteristic biochemical or physiological effect.

Signaling processes may elicit a variety of effects on cells and tissues including, by way of nonlimiting example, induction of cell or tissue proliferation, suppression of growth or proliferation, induction of differentiation or maturation of a cell or tissue, and suppression of differentiation or maturation of a cell or tissue.

Many pathological conditions involve dysregulation of expression of important effector proteins. In certain classes of pathologies the dysregulation is manifested as diminished or suppressed level of synthesis and secretion of protein effectors. In other classes of pathologies the dysregulation is manifested as increased or up-regulated level of synthesis and secretion of protein effectors. In a clinical setting a subject may be suspected of suffering from a condition brought on by altered or mis-regulated levels of a protein effector of interest. Therefore there is a need to assay for the level of the protein effector of interest in a biological sample from such a subject, and to compare the level with that characteristic of a nonpathological condition. There also is a need to provide the protein effector as a product of manufacture. Administration of the effector to a subject in need thereof is useful in treatment of the pathological condition. Accordingly, there is a need for a method of treatment of a pathological condition brought on by a diminished or suppressed levels of the protein effector of interest. In addition, there is a need for a method of treatment of a pathological condition brought on by a increased or up-regulated levels of the protein effector of interest.

Antibodies are multichain proteins that bind specifically to a given antigen, and bind poorly, or not at all, to substances deemed not to be cognate antigens. Antibodies are comprised of two short chains termed light chains and two long chains termed heavy chains. These chains are constituted of immunoglobulin domains, of which generally there are two classes: one variable domain per chain, one constant domain in light chains, and three or more constant domains in heavy chains. The antigen-specific portion of the immunoglobulin molecules resides in the variable domains; the variable domains of one light chain and one heavy chain associate with each other to generate the antigen-binding moiety. Antibodies that bind immunospecifically to a cognate or target antigen bind with high affinities. Accordingly, they are useful in assaying specifically for the presence of the antigen in a sample. In addition, they have the potential of inactivating the activity of the antigen.

Therefore there is a need to assay for the level of a protein effector of interest in a biological sample from such a subject, and to compare this level with that characteristic of a nonpathological condition. In particular, there is a need for such an assay based on the use of an antibody that binds immunospecifically to the antigen. There further is a need to inhibit the activity of the protein effector in cases where a pathological condition arises from elevated or excessive levels of the effector based on the use of an antibody that binds immunospecifically to the effector. Thus, there is a need for the antibody as a product of manufacture. There further is a need for a method of treatment of a pathological condition brought on by an elevated or excessive level of the protein effector of interest based on administering the antibody to the subject.

### SUMMARY OF THE INVENTION

The invention is based in part upon the discovery of isolated polypeptides including amino acid sequences selected from mature forms of the amino acid sequences selected from the group consisting of SEQ ID NO:2n, wherein n is an integer between 1 and 141. The novel nucleic acids and polypeptides are referred to herein as NOVX, or NOVI, NOV2, NOV3, etc., nucleic acids and polypeptides. These nucleic acids and polypeptides, as well as derivatives, homologs, analogs and fragments thereof, will hereinafter be collectively designated as “NOVX” nucleic acid or polypeptide sequences.

The invention also is based in part upon variants of a mature form of the amino acid sequence selected from the group consisting of SEQ ID NO:2n, wherein n is an integer between 1 and 141, wherein any amino acid in the mature form is changed to a different amino acid, provided that no more than 15% of the amino acid residues in the sequence of the mature form are so changed. In another embodiment, the invention includes the amino acid sequences selected from the group consisting of SEQ ID NO:2n, wherein n is an integer between 1 and 141. In another embodiment, the invention also comprises variants of the amino acid sequence selected from the group consisting of SEQ ID NO:2n, wherein n is an integer between 1 and 141, wherein any amino acid specified in the chosen sequence is changed to a different amino acid, provided that no more than 15% of the amino acid residues in the sequence are so changed. The invention also involves fragments of any of the mature forms of the amino acid sequences selected from the group consisting of SEQ ID NO:2n, wherein n is an integer between 1 and 141, or any other amino acid sequence selected from this group. The invention also comprises fragments from these groups in which up to 15% of the residues are changed.

In another embodiment, the invention encompasses polypeptides that are naturally occurring allelic variants of the sequence selected from the group consisting of SEQ ID NO:2n, wherein n is an integer between 1 and 141. These allelic variants include amino acid sequences that are the translations of nucleic acid sequences differing by a single nucleotide from nucleic acid sequences selected from the group consisting of SEQ ID NOS: 2n−1, wherein n is an integer between 1 and 141. The variant polypeptide where any amino acid changed in the chosen sequence is changed to provide a conservative substitution.

In another embodiment, the invention comprises a pharmaceutical composition involving a polypeptide with an amino acid sequence selected from the group consisting of SEQ ID NO:2n, wherein n is an integer between 1 and 141, and a pharmaceutically acceptable carrier. In another embodiment, the invention involves a kit, including, in one or more containers, this pharmaceutical composition.

In another embodiment, the invention includes the use of a therapeutic in the manufacture of a medicament for treating a syndrome associated with a human disease, the disease being selected from a pathology associated with a polypeptide with an amino acid sequence selected from the group consisting of SEQ ID NO:2n, wherein n is an integer between 1 and 141, wherein said therapeutic is the polypeptide selected from this group.

In another embodiment, the invention comprises a method for determining the presence or amount of a polypeptide with an amino acid sequence selected from the group consisting of SEQ ID NO:2n, wherein n is an integer between 1 and 141, in a sample, the method involving providing the sample; introducing the sample to an antibody that binds immunospecifically to the polypeptide; and determining the presence or amount of antibody bound to the polypeptide, thereby determining the presence or amount of polypeptide in the sample.

In another embodiment, the invention includes a method for determining the presence of or predisposition to a disease associated with altered levels of a polypeptide with an amino acid sequence selected from the group consisting of SEQ ID NO:2n, wherein n is an integer between 1 and 141, in a first mammalian subject, the method involving measuring the level of expression of the polypeptide in a sample from the first mammalian subject; and comparing the amount of the polypeptide in this sample to the amount of the polypeptide present in a control sample from a second mammalian subject known not to have, or not to be predisposed to, the disease, wherein an alteration in the expression level of the polypeptide in the first subject as compared to the control sample indicates the presence of or predisposition to the disease.

In another embodiment, the invention involves a method of identifying an agent that binds to a polypeptide with an amino acid sequence selected from the group consisting of SEQ ID NO:2n, wherein n is an integer between 1 and 141, the method including introducing the polypeptide to the agent; and determining whether the agent binds to the polypeptide. The agent could be a cellular receptor or a downstream effector.

In another embodiment, the invention involves a method for identifying a potential therapeutic agent for use in treatment of a pathology, wherein the pathology is related to aberrant expression or aberrant physiological interactions of a polypeptide with an amino acid sequence selected from the group consisting of SEQ ID NO:2n, wherein n is an integer between 1 and 141, the method including providing a cell expressing the polypeptide of the invention and having a property or function ascribable to the polypeptide; contacting the cell with a composition comprising a candidate substance; and determining whether the substance alters the property or function ascribable to the polypeptide; whereby, if an alteration observed in the presence of the substance is not observed when the cell is contacted with a composition devoid of the substance, the substance is identified as a potential therapeutic agent.

In another embodiment, the invention involves a method for screening for a modulator of activity or of latency or predisposition to a pathology associated with a polypeptide having an amino acid sequence selected from the group consisting of SEQ ID NO:2n, wherein n is an integer between 1 and 141, the method including administering a test compound to a test animal at increased risk for a pathology associated with the polypeptide of the invention, wherein the test animal recombinantly expresses the polypeptide of the invention; measuring the activity of the polypeptide in the test animal after administering the test compound; and comparing the activity of the protein in the test animal with the activity of the polypeptide in a control animal not administered the polypeptide, wherein a change in the activity of the polypeptide in the test animal relative to the control animal indicates the test compound is a modulator of latency of, or predisposition to, a pathology associated with the polypeptide of the invention. The recombinant test animal could express a test protein transgene or express the transgene under the control of a promoter at an increased level relative to a wild-type test animal The promoter may or may not b the native gene promoter of the transgene.

In another embodiment, the invention involves a method for modulating the activity of a polypeptide with an amino acid sequence selected from the group consisting of SEQ ID NO:2n, wherein n is an integer between 1 and 141, the method including introducing a cell sample expressing the polypeptide with a compound that binds to the polypeptide in an amount sufficient to modulate the activity of the polypeptide.

In another embodiment, the invention involves a method of treating or preventing a pathology associated with a polypeptide with an amino acid sequence selected from the group consisting of SEQ ID NO:2n, wherein n is an integer between 1 and 141, the method including administering the polypeptide to a subject in which such treatment or prevention is desired in an amount sufficient to treat or prevent the pathology in the subject. The subject could be human.

In another embodiment, the invention involves a method of treating a pathological state in a mammal, the method including administering to the mammal a polypeptide in an amount that is sufficient to alleviate the pathological state, wherein the polypeptide is a polypeptide having an amino acid sequence at least 95% identical to a polypeptide having the amino acid sequence selected from the group consisting of SEQ ID NO:2n, wherein n is an integer between 1 and 141, or a biologically active fragment thereof.

In another embodiment, the invention involves an isolated nucleic acid molecule comprising a nucleic acid sequence encoding a polypeptide having an amino acid sequence selected from the group consisting of a mature form of the amino acid sequence given SEQ ID NO:2n, wherein n is an integer between 1 and 141, a variant of a mature form of the amino acid sequence selected from the group consisting of SEQ ID NO:2n, wherein n is an integer between 1 and 141, wherein any amino acid in the mature form of the chosen sequence is changed to a different amino acid, provided that no more than 15% of the amino acid residues in the sequence of the mature form are so changed; the amino acid sequence selected from the group consisting of SEQ ID NO:2n, wherein n is an integer between 1 and 141, a variant of the amino acid sequence selected from the group consisting of SEQ ID NO:2n, wherein n is an integer between 1 and 141, in which any amino acid specified in the chosen sequence is changed to a different amino acid, provided that no more than 15% of the amino acid residues in the sequence are so changed; a nucleic acid fragment encoding at least a portion of a polypeptide comprising the amino acid sequence selected from the group consisting of SEQ ID NO:2n, wherein n is an integer between 1 and 141, or any variant of the polypeptide wherein any amino acid of the chosen sequence is changed to a different amino acid, provided that no more than 10% of the amino acid residues in the sequence are so changed; and the complement of any of the nucleic acid molecules.

In another embodiment, the invention comprises an isolated nucleic acid molecule having a nucleic acid sequence encoding a polypeptide comprising an amino acid sequence selected from the group consisting of a mature form of the amino acid sequence given SEQ ID NO:2n, wherein n is an integer between 1 and 141, wherein the nucleic acid molecule comprises the nucleotide sequence of a naturally occurring allelic nucleic acid variant.

In another embodiment, the invention involves an isolated nucleic acid molecule including a nucleic acid sequence encoding a polypeptide having an amino acid sequence selected from the group consisting of a mature form of the amino acid sequence given SEQ ID NO:2n, wherein n is an integer between 1 and 141, that encodes a variant polypeptide, wherein the variant polypeptide has the polypeptide sequence of a naturally occurring polypeptide variant.

In another embodiment, the invention comprises an isolated nucleic acid molecule having a nucleic acid sequence encoding a polypeptide comprising an amino acid sequence selected from the group consisting of a mature form of the amino acid sequence given SEQ ID NO:2n, wherein n is an integer between 1 and 141, wherein the nucleic acid molecule differs by a single nucleotide from a nucleic acid sequence selected from the group consisting of SEQ ID NOS: 2n−1, wherein n is an integer between 1 and 141.

In another embodiment, the invention includes an isolated nucleic acid molecule having a nucleic acid sequence encoding a polypeptide including an amino acid sequence selected from the group consisting of a mature form of the amino acid sequence given SEQ ID NO:2n, wherein n is an integer between 1 and 141, wherein the nucleic acid molecule comprises a nucleotide sequence selected from the group consisting of the nucleotide sequence selected from the group consisting of SEQ ID NO:2n−1, wherein n is an integer between 1 and 141, a nucleotide sequence wherein one or more nucleotides in the nucleotide sequence selected from the group consisting of SEQ ID NO:2n−1, wherein n is an integer between 1 and 141, is changed from that selected from the group consisting of the chosen sequence to a different nucleotide provided that no more than 15% of the nucleotides are so changed; a nucleic acid fragment of the sequence selected from the group consisting of SEQ ID NO:2n−1, wherein n is an integer between 1 and 141, and a nucleic acid fragment wherein one or more nucleotides in the nucleotide sequence selected from the group consisting of SEQ ID NO:2n−1, wherein n is an integer between 1 and 141, is changed from that selected from the group consisting of the chosen sequence to a different nucleotide provided that no more than 15% of the nucleotides are so changed.

In another embodiment, the invention includes an isolated nucleic acid molecule having a nucleic acid sequence encoding a polypeptide including an amino acid sequence selected from the group consisting of a mature form of the amino acid sequence given SEQ ID NO:2n, wherein n is an integer between 1 and 141, wherein the nucleic acid molecule hybridizes under stringent conditions to the nucleotide sequence selected from the group consisting of SEQ ID NO:2n−1, wherein n is an integer between 1 and 141, or a complement of the nucleotide sequence.

In another embodiment, the invention includes an isolated nucleic acid molecule having a nucleic acid sequence encoding a polypeptide including an amino acid sequence selected from the group consisting of a mature form of the amino acid sequence given SEQ ID NO:2n, wherein n is an integer between 1 and 141, wherein the nucleic acid molecule has a nucleotide sequence in which any nucleotide specified in the coding sequence of the chosen nucleotide sequence is changed from that selected from the group consisting of the chosen sequence to a different nucleotide provided that no more than 15% of the nucleotides in the chosen coding sequence are so changed, an isolated second polynucleotide that is a complement of the first polynucleotide, or a fragment of any of them.

In another embodiment, the invention includes a vector involving the nucleic acid molecule having a nucleic acid sequence encoding a polypeptide including an amino acid sequence selected from the group consisting of a mature form of the amino acid sequence given SEQ ID NO:2n, wherein n is an integer between 1 and 141. This vector can have a promoter operably linked to the nucleic acid molecule. This vector can be located within a cell.

In another embodiment, the invention involves a method for determining the presence or amount of a nucleic acid molecule having a nucleic acid sequence encoding a polypeptide including an amino acid sequence selected from the group consisting of a mature form of the amino acid sequence given SEQ ID NO:2n, wherein n is an integer between 1 and 141, in a sample, the method including providing the sample; introducing the sample to a probe that binds to the nucleic acid molecule; and determining the presence or amount of the probe bound to the nucleic acid molecule, thereby determining the presence or amount of the nucleic acid molecule in the sample. The presence or amount of the nucleic acid molecule is used as a marker for cell or tissue type. The cell type can be cancerous.

In another embodiment, the invention involves a method for determining the presence of or predisposition for a disease associated with altered levels of a nucleic acid molecule having a nucleic acid sequence encoding a polypeptide including an amino acid sequence selected from the group consisting of a mature form of the amino acid sequence given SEQ ID NO:2n, wherein n is an integer between 1 and 141, in a first mammalian subject, the method including measuring the amount of the nucleic acid in a sample from the first mammalian subject; and comparing the amount of the nucleic acid in the sample of step (a) to the amount of the nucleic acid present in a control sample from a second mammalian subject known not to have or not be predisposed to, the disease; wherein an alteration in the level of the nucleic acid in the first subject as compared to the control sample indicates the presence of or predisposition to the disease.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed description and claims.

### DETAILED DESCRIPTION OF THE INVENTION

The present invention provides novel nucleotides and polypeptides encoded thereby. Included in the invention are the novel nucleic acid sequences, their encoded polypeptides, antibodies, and other related compounds. The sequences are collectively referred to herein as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table A provides a summary of the NOVX nucleic acids and their encoded polypeptides.

TABLE A
Sequences and Corresponding SEQ ID Numbers

| | | SEQ ID | SEQ ID | |
|---|---|---|---|---|
| | | NO | NO | |
| NOVX | Internal | (nucleic | (amino | |
| Assignment | Identification | acid) | acid) | Homology |
| NOV1a | CG103945-02 | 1 | 2 | Semaphorin sem2 (FLJ00014 protein) - |
| | | | | Homo sapiens |
| NOV1b | CG103945-01 | 3 | 4 | Semaphorin sem2 (FLJ00014 protein) - |
| | | | | Homo sapiens |
| NOV2a | CG106951-01 | 5 | 6 | Human semaphorin G-like NHP protein |
| NOV2b | CG106951-04 | 7 | 8 | Human semaphorin G-like NHP protein |
| NOV2c | 209829549 | 9 | 10 | Human semaphorin G-like NHP protein |
| NOV2d | 209829553 | 11 | 12 | Human semaphorin G-like NHP protein |
| NOV2e | 209829642 | 13 | 14 | Human semaphorin G-like NHP protein |
| NOV2f | 209829670 | 15 | 16 | Human semaphorin G-like NHP protein |
| NOV2g | CG106951-02 | 17 | 18 | Human semaphorin G-like NHP protein |
| NOV2h | CG106951-03 | 19 | 20 | Human semaphorin G-like NHP protein |
| NOV2i | SNP13382456 | 21 | 22 | Human semaphorin G-like NHP protein |
| NOV3a | CG121295-01 | 23 | 24 | Endothelin-1 precursor (ET-1) - Homo |
| | | | | sapiens |
| NOV4a | CG124756-01 | 25 | 26 | complement subcomponent C1q chain |
| | | | | B precursor [validated] |
| NOV4b | CG124756-02 | 27 | 28 | complement subcomponent C1q chain |
| | | | | B precursor [validated] |
| NOV4c | SNP13382475 | 29 | 30 | complement subcomponent C1q chain |
| | | | | B precursor [validated] |
| NOV4d | SNP13382476 | 31 | 32 | complement subcomponent C1q chain |
| | | | | B precursor [validated] |
| NOV5a | CG50353-01 | 33 | 34 | Wnt-7a protein precursor - Homo |
| | | | | sapiens |
| NOV5b | 228753443 | 35 | 36 | Wnt-7a protein precursor - Homo |
| | | | | sapiens |
| NOV5c | 169475673 | 37 | 38 | Wnt-7a protein precursor - Homo |
| | | | | sapiens |
| NOV5d | 228753459 | 39 | 40 | Wnt-7a protein precursor - Homo |
| | | | | sapiens |
| NOV5e | 228753462 | 41 | 42 | Wnt-7a protein precursor - Homo |
| | | | | sapiens |
| NOV5f | 228753446 | 43 | 44 | Wnt-7a protein precursor - Homo |
| | | | | sapiens |
| NOV5g | 228753465 | 45 | 46 | Wnt-7a protein precursor - Homo |
| | | | | sapiens |
| NOV5h | 228753438 | 47 | 48 | Wnt-7a protein precursor - Homo |
| | | | | sapiens |
| NOV5i | 228753449 | 49 | 50 | Wnt-7a protein precursor - Homo |
| | | | | sapiens |
| NOV5j | CG50353-02 | 51 | 52 | Wnt-7a protein precursor - Homo |
| | | | | sapiens |
| NOV5k | CG50353-03 | 53 | 54 | Wnt-7a protein precursor - Homo |
| | | | | sapiens |
| NOV5l | SNP13382474 | 55 | 56 | Wnt-7a protein precursor - Homo |
| | | | | sapiens |
| NOV6a | CG50709-03 | 57 | 58 | Wnt-9b protein precursor (Wnt-15) |
| | | | | (Wnt-14b) - Homo sapiens |
| NOV6b | 282997951 | 59 | 60 | Wnt-9b protein precursor (Wnt-15) |
| | | | | (Wnt-14b) - Homo sapiens |
| NOV6c | CG50709-05 | 61 | 62 | Wnt-9b protein precursor (Wnt-15) |
| | | | | (Wnt-14b) - Homo sapiens |
| NOV6d | 277582109 | 63 | 64 | Wnt-9b protein precursor (Wnt-15) |
| | | | | (Wnt-14b) - Homo sapiens |
| NOV6e | 277582117 | 65 | 66 | Wnt-9b protein precursor (Wnt-15) |
| | | | | (Wnt-14b) - Homo sapiens |
| NOV6f | CG50709-01 | 67 | 68 | Wnt-9b protein precursor (Wnt-15) |
| | | | | (Wnt-14b) - Homo sapiens |
| NOV6g | CG50709-02 | 69 | 70 | Wnt-9b protein precursor (Wnt-15) |
| | | | | (Wnt-14b) - Homo sapiens |
| NOV6h | CG50709-04 | 71 | 72 | Wnt-9b protein precursor (Wnt-15) |
| | | | | (Wnt-14b) - Homo sapiens |
| NOV6i | CG50709-06 | 73 | 74 | Wnt-9b protein precursor (Wnt-15) |
| | | | | (Wnt-14b) - Homo sapiens |
| NOV6j | CG50709-07 | 75 | 76 | Wnt-9b protein precursor (Wnt-15) |
| | | | | (Wnt-14b) - Homo sapiens |
| NOV6k | SNP13381605 | 77 | 78 | Wnt-9b protein precursor (Wnt-15) |
| | | | | (Wnt-14b) - Homo sapiens |
| NOV6l | SNP13381606 | 79 | 80 | Wnt-9b protein precursor (Wnt-15) |
| | | | | (Wnt-14b) - Homo sapiens |
| NOV6m | SNP13378337 | 81 | 82 | Wnt-9b protein precursor (Wnt-15) |
| | | | | (Wnt-14b) - Homo sapiens |
| NOV6n | SNP13381607 | 83 | 84 | Wnt-9b protein precursor (Wnt-15) |
| | | | | (Wnt-14b) - Homo sapiens |
| NOV6o | SNP13378336 | 85 | 86 | Wnt-9b protein precursor (Wnt-15) |
| | | | | (Wnt-14b) - Homo sapiens |
| NOV6p | SNP13378335 | 87 | 88 | Wnt-9b protein precursor (Wnt-15) |
| | | | | (Wnt-14b) - Homo sapiens |
| NOV7a | CG53054-02 | 89 | 90 | Wnt-9a protein precursor (Wnt-14) - |
| | | | | Homo sapiens |
| NOV7b | 170251039 | 91 | 92 | Wnt-9a protein precursor (Wnt-14) - |
| | | | | Homo sapiens |
| NOV7c | 170251076 | 93 | 94 | Wnt-9a protein precursor (Wnt-14) - |
| | | | | Homo sapiens |
| NOV7d | CG53054-01 | 95 | 96 | Wnt-9a protein precursor (Wnt-14) - |
| | | | | Homo sapiens |
| NOV7e | CG53054-03 | 97 | 98 | Wnt-9a protein precursor (Wnt-14) - |
| | | | | Homo sapiens |
| NOV7f | CG53054-04 | 99 | 100 | Wnt-9a protein precursor (Wnt-14) - |
| | | | | Homo sapiens |
| NOV8a | CG53473-02 | 101 | 102 | Neuromedin B-32 precursor [Contains: |
| | | | | Neuromedin B] - Homo sapiens |
| NOV8b | CG53473-01 | 103 | 104 | Neuromedin B-32 precursor [Contains: |
| | | | | Neuromedin B] - Homo sapiens |
| NOV8c | CG53473-03 | 105 | 106 | Neuromedin B-32 precursor [Contains: |
| | | | | Neuromedin B] - Homo sapiens |
| NOV8d | SNP13376396 | 107 | 108 | Neuromedin B-32 precursor [Contains: |
| | | | | Neuromedin B] - Homo sapiens |
| NOV8e | SNP13376395 | 109 | 110 | Neuromedin B-32 precursor [Contains: |
| | | | | Neuromedin B] - Homo sapiens |
| NOV8f | SNP13376394 | 111 | 112 | Neuromedin B-32 precursor [Contains: |
| | | | | Neuromedin B] - Homo sapiens |
| NOV9a | CG55184-03 | 113 | 114 | Cerebellin-like glycoprotein 1 precursor - |
| | | | | Homo sapiens |
| NOV9b | CG55184-01 | 115 | 116 | Cerebellin-like glycoprotein 1 precursor - |
| | | | | Homo sapiens |
| NOV9c | CG55184-02 | 117 | 118 | Cerebellin-like glycoprotein 1 precursor - |
| | | | | Homo sapiens |
| NOV9d | CG55184-04 | 119 | 120 | Cerebellin-like glycoprotein 1 precursor - |
| | | | | Homo sapiens |
| NOV9e | CG55184-05 | 121 | 122 | Cerebellin-like glycoprotein 1 precursor - |
| | | | | Homo sapiens |
| NOV10a | CG55274-05 | 123 | 124 | Human endozepine-like ENDO5 |
| NOV10b | CG55274-01 | 125 | 126 | Human endozepine-like ENDO5 |
| NOV10c | CG55274-02 | 127 | 128 | Human endozepine-like ENDO5 |
| NOV10d | CG55274-03 | 129 | 130 | Human endozepine-like ENDO5 |
| NOV10e | CG55274-04 | 131 | 132 | Human endozepine-like ENDO5 |
| NOV11a | CG55379-04 | 133 | 134 | HDDM36 - Homo sapiens |
| NOV11b | CG55379-01 | 135 | 136 | HDDM36 - Homo sapiens |
| NOV11c | 258065951 | 137 | 138 | HDDM36 - Homo sapiens |
| NOV11d | 209886264 | 139 | 140 | HDDM36 - Homo sapiens |
| NOV11e | 209886345 | 141 | 142 | HDDM36 - Homo sapiens |
| NOV11f | 209886357 | 143 | 144 | HDDM36 - Homo sapiens |
| NOV11g | CG55379-02 | 145 | 146 | HDDM36 - Homo sapiens |
| NOV11h | CG55379-03 | 147 | 148 | HDDM36 - Homo sapiens |
| NOV12a | CG55688-01 | 149 | 150 | CYR61 protein precursor |
| | | | | (Cysteine-rich, angiogenic inducer, 61) |
| | | | | (Insulin-like growth factor-binding |
| | | | | protein 10) (GIG1 protein) - Homo |
| | | | | sapiens |
| NOV12b | 254087906 | 151 | 152 | CYR61 protein precursor |
| | | | | (Cysteine-rich, angiogenic inducer, 61) |
| | | | | (Insulin-like growth factor-binding |
| | | | | protein 10) (GIG1 protein) - Homo |
| | | | | sapiens |
| NOV12c | 259278648 | 153 | 154 | CYR61 protein precursor |
| | | | | (Cysteine-rich, angiogenic inducer, 61) |
| | | | | (Insulin-like growth factor-binding |
| | | | | protein 10) (GIG1 protein) - Homo |
| | | | | sapiens |
| NOV12d | 259280032 | 155 | 156 | CYR61 protein precursor |
| | | | | (Cysteine-rich, angiogenic inducer, 61) |
| | | | | (Insulin-like growth factor-binding |
| | | | | protein 10) (GIG1 protein) - Homo |
| | | | | sapiens |
| NOV12e | 254756530 | 157 | 158 | CYR61 protein precursor |
| | | | | (Cysteine-rich, angiogenic inducer, 61) |
| | | | | (Insulin-like growth factor-binding |
| | | | | protein 10) (GIG1 protein) - Homo |
| | | | | sapiens |
| NOV12f | 229509618 | 159 | 160 | CYR61 protein precursor |
| | | | | (Cysteine-rich, angiogenic inducer, 61) |
| | | | | (Insulin-like growth factor-binding |
| | | | | protein 10) (GIG1 protein) - Homo |
| | | | | sapiens |
| NOV12g | 229509658 | 161 | 162 | CYR61 protein precursor |
| | | | | (Cysteine-rich, angiogenic inducer, 61) |
| | | | | (Insulin-like growth factor-binding |
| | | | | protein 10) (GIG1 protein) - Homo |
| | | | | sapiens |
| NOV12h | CG55688-02 | 163 | 164 | CYR61 protein precursor |
| | | | | (Cysteine-rich, angiogenic inducer, 61) |
| | | | | (Insulin-like growth factor-binding |
| | | | | protein 10) (GIG1 protein) - Homo |
| | | | | sapiens |
| NOV12i | CG55688-03 | 165 | 166 | CYR61 protein precursor |
| | | | | (Cysteine-rich, angiogenic inducer, 61) |
| | | | | (Insulin-like growth factor-binding |
| | | | | protein 10) (GIG1 protein) - Homo |
| | | | | sapiens |
| NOV12j | CG55688-04 | 167 | 168 | CYR61 protein precursor |
| | | | | (Cysteine-rich, angiogenic inducer, 61) |
| | | | | (Insulin-like growth factor-binding |
| | | | | protein 10) (GIG1 protein) - Homo |
| | | | | sapiens |
| NOV12k | CG55688-05 | 169 | 170 | CYR61 protein precursor |
| | | | | (Cysteine-rich, angiogenic inducer, 61) |
| | | | | (Insulin-like growth factor-binding |
| | | | | protein 10) (GIG1 protein) - Homo |
| | | | | sapiens |
| NOV12l | CG55688-06 | 171 | 172 | CYR61 protein precursor |
| | | | | (Cysteine-rich, angiogenic inducer, 61) |
| | | | | (Insulin-like growth factor-binding |
| | | | | protein 10) (GIG1 protein) - Homo |
| | | | | sapiens |
| NOV12m | SNP13376428 | 173 | 174 | CYR61 protein precursor |
| | | | | (Cysteine-rich, angiogenic inducer, 61) |
| | | | | (Insulin-like growth factor-binding |
| | | | | protein 10) (GIG1 protein) - Homo |
| | | | | sapiens |
| NOV13a | CG56768-01 | 175 | 176 | Wnt-5a protein precursor - Homo |
| | | | | sapiens |
| NOV13b | CG56768-02 | 177 | 178 | Wnt-5a protein precursor - Homo |
| | | | | sapiens |
| NOV13c | CG56768-03 | 179 | 180 | Wnt-5a protein precursor - Homo |
| | | | | sapiens |
| NOV14a | CG57054-03 | 181 | 182 | Wnt-10b protein precursor (Wnt-12) - |
| | | | | Homo sapiens |
| NOV14b | CG57054-01 | 183 | 184 | Wnt-10b protein precursor (Wnt-12) - |
| | | | | Homo sapiens |
| NOV14c | CG57054-02 | 185 | 186 | Wnt-10b protein precursor (Wnt-12) - |
| | | | | Homo sapiens |
| NOV15a | CG57431-03 | 187 | 188 | Endothelin-2 precursor (ET-2) - Homo |
| | | | | sapiens |
| NOV15b | CG57431-02 | 189 | 190 | Endothelin-2 precursor (ET-2) - Homo |
| | | | | sapiens |
| NOV15c | CG57431-01 | 191 | 192 | Endothelin-2 precursor (ET-2) - Homo |
| | | | | sapiens |
| NOV15d | CG57431-04 | 193 | 194 | Endothelin-2 precursor (ET-2) - Homo |
| | | | | sapiens |
| NOV16a | CG59253-01 | 195 | 196 | Semaphorin 6D isoform 2 - Homo |
| | | | | sapiens |
| NOV16b | 194877881 | 197 | 198 | Semaphorin 6D isoform 2 - Homo |
| | | | | sapiens |
| NOV16c | CG59253-02 | 199 | 200 | Semaphorin 6D isoform 2 - Homo |
| | | | | sapiens |
| NOV16d | 191815765 | 201 | 202 | Semaphorin 6D isoform 2 - Homo |
| | | | | sapiens |
| NOV16e | CG59253-03 | 203 | 204 | Semaphorin 6D isoform 2 - Homo |
| | | | | sapiens |
| NOV16f | CG59253-04 | 205 | 206 | Semaphorin 6D isoform 2 - Homo |
| | | | | sapiens |
| NOV16g | CG59253-05 | 207 | 208 | Semaphorin 6D isoform 2 - Homo |
| | | | | sapiens |
| NOV16h | CG59253-06 | 209 | 210 | Semaphorin 6D isoform 2 - Homo |
| | | | | sapiens |
| NOV16i | CG59253-07 | 211 | 212 | Semaphorin 6D isoform 2 - Homo |
| | | | | sapiens |
| NOV16j | CG59253-08 | 213 | 214 | Semaphorin 6D isoform 2 - Homo |
| | | | | sapiens |
| NOV16k | CG59253-09 | 215 | 216 | Semaphorin 6D isoform 2 - Homo |
| | | | | sapiens |
| NOV16l | CG59253-10 | 217 | 218 | Semaphorin 6D isoform 2 - Homo |
| | | | | sapiens |
| NOV16m | SNP13381547 | 219 | 220 | Semaphorin 6D isoform 2 - Homo |
| | | | | sapiens |
| NOV16n | SNP13378936 | 221 | 222 | Semaphorin 6D isoform 2 - Homo |
| | | | | sapiens |
| NOV16o | SNP13378935 | 223 | 224 | Semaphorin 6D isoform 2 - Homo |
| | | | | sapiens |
| NOV16p | SNP13381569 | 225 | 226 | Semaphorin 6D isoform 2 - Homo |
| | | | | sapiens |
| NOV16q | SNP13382528 | 227 | 228 | Semaphorin 6D isoform 2 - Homo |
| | | | | sapiens |
| NOV17a | CG95430-02 | 229 | 230 | Energen-related secreted protein - C2P |
| NOV17b | CG95430-04 | 231 | 232 | Energen-related secreted protein - C2P |
| NOV17c | CG95430-01 | 233 | 234 | Energen-related secreted protein - C2P |
| NOV17d | 319194717 | 235 | 236 | Energen-related secreted protein - C2P |
| NOV17e | CG95430-03 | 237 | 238 | Energen-related secreted protein - C2P |
| NOV17f | CG95430-05 | 239 | 240 | Energen-related secreted protein - C2P |
| NOV17g | CG95430-06 | 241 | 242 | Energen-related secreted protein - C2P |
| NOV17h | CG95430-07 | 243 | 244 | Energen-related secreted protein - C2P |
| NOV17i | CG95430-08 | 245 | 246 | Energen-related secreted protein - C2P |
| NOV17j | CG95430-09 | 247 | 248 | Energen-related secreted protein - C2P |
| NOV17k | CG95430-10 | 249 | 250 | Energen-related secreted protein - C2P |
| NOV17l | CG95430-11 | 251 | 252 | Energen-related secreted protein - C2P |
| NOV17m | CG95430-12 | 253 | 254 | Energen-related secreted protein - C2P |
| NOV17n | CG95430-13 | 255 | 256 | Energen-related secreted protein - C2P |
| NOV17o | SNP13379412 | 257 | 258 | Energen-related secreted protein - C2P |
| NOV17p | SNP13381828 | 259 | 260 | Energen-related secreted protein - C2P |
| NOV17q | SNP13379125 | 261 | 262 | Energen-related secreted protein - C2P |
| NOV17r | SNP13381827 | 263 | 264 | Energen-related secreted protein - C2P |
| NOV17s | SNP13381822 | 265 | 266 | Energen-related secreted protein - C2P |
| NOV17t | SNP13381826 | 267 | 268 | Energen-related secreted protein - C2P |
| NOV18a | CG97111-01 | 269 | 270 | Human IL-1 receptor antagonist protein |
| NOV18b | CG97111-02 | 271 | 272 | Human IL-1 receptor antagonist protein |
| NOV18c | CG97111-03 | 273 | 274 | Human IL-1 receptor antagonist protein |
| NOV18d | SNP13382516 | 275 | 276 | Human IL-1 receptor antagonist protein |
| NOV18e | SNP13382517 | 277 | 278 | Human IL-1 receptor antagonist protein |
| NOV18f | SNP13382518 | 279 | 280 | Human IL-1 receptor antagonist protein |
| NOV19a | 10132038.0.67 | 281 | 282 | Domain of CG50513-05 |

Table A indicates the homology of NOVX polypeptides to known protein families. Thus, the nucleic acids and polypeptides, antibodies and related compounds according to the invention corresponding to a NOVX as identified in column 1 of Table A will be useful in therapeutic and diagnostic applications implicated in, for example, pathologies and disorders associated with the known protein families identified in column 5 of Table A.

Pathologies, diseases, disorders, conditions and the like that are associated with NOVX sequences include, but are not limited to, e.g., cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, metabolic disturbances associated with obesity, transplantation, adrenoleukodystrophy, congenital adrenal hyperplasia, prostate cancer, diabetes, metabolic disorders, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, infectious disease, anorexia, cancer-associated cachexia, cancer, neurodegenerative disorders, Alzheimer's Disease, Parkinson's Disorder, immune disorders, hematopoietic disorders, and the various dyslipidemias, the metabolic syndrome X and wasting disorders associated with chronic diseases and various cancers, as well as conditions such as transplantation and fertility.

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

Consistent with other known members of the family of proteins, identified in column 5 of Table A, the NOVX polypeptides of the present invention show homology to, and contain domains that are characteristic of, other members of such protein families. Details of the sequence relatedness and domain analysis for each NOVX are presented in Example A.

The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit diseases associated with the protein families listed in Table A.

The NOVX nucleic acids and polypeptides are also useful for detecting specific cell types. Details of the expression analysis for each NOVX are presented in Example C. Accordingly, the NOVX nucleic acids, polypeptides, antibodies and related compounds according to the invention will have diagnostic and therapeutic applications in the detection of a variety of diseases with differential expression in normal vs. diseased tissues, e.g., detection of a variety of cancers.

Additional utilities for NOVX nucleic acids and polypeptides according to the invention are disclosed herein.

NOVX Clones

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

The NOVX genes and their corresponding encoded proteins are useful for preventing, treating or ameliorating medical conditions, e.g., by protein or gene therapy. Pathological conditions can be diagnosed by determining the amount of the new protein in a sample or by determining the presence of mutations in the new genes. Specific uses are described for each of the NOVX genes, based on the tissues in which they are most highly expressed. Uses include developing products for the diagnosis or treatment of a variety of diseases and disorders.

The NOVX nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) a biological defense weapon.

In one specific embodiment, the invention includes an isolated polypeptide comprising an amino acid sequence selected from the group consisting of: (a) a mature form of the amino acid sequence selected from the group consisting of SEQ ID NO:2n, wherein n is an integer between 1 and 141, (b) a variant of a mature form of the amino acid sequence selected from the group consisting of SEQ ID NO:2n, wherein n is an integer between 1 and 141, wherein any amino acid in the mature form is changed to a different amino acid, provided that no more than 15% of the amino acid residues in the sequence of the mature form are so changed; (c) an amino acid sequence selected from the group consisting of SEQ ID NO:2n, wherein n is an integer between 1 and 141, (d) a variant of the amino acid sequence selected from the group consisting of SEQ ID NO:2n, wherein n is an integer between 1 and 141, wherein any amino acid specified in the chosen sequence is changed to a different amino acid, provided that no more than 15% of the amino acid residues in the sequence are so changed; and (e) a fragment of any of (a) through (d).

In another specific embodiment, the invention includes an isolated nucleic acid molecule comprising a nucleic acid sequence encoding a polypeptide comprising an amino acid sequence selected from the group consisting of: (a) a mature form of the amino acid sequence given SEQ ID NO:2n, wherein n is an integer between 1 and 141; (b) a variant of a mature form of the amino acid sequence selected from the group consisting of SEQ ID NO:2n, wherein n is an integer between 1 and 141, wherein any amino acid in the mature form of the chosen sequence is changed to a different amino acid, provided that no more than 15% of the amino acid residues in the sequence of the mature form are so changed; (c) the amino acid sequence selected from the group consisting of SEQ ID NO:2n, wherein n is an integer between 1 and 141; (d) a variant of the amino acid sequence selected from the group consisting of SEQ ID NO:2n, wherein n is an integer between 1 and 141, in which any amino acid specified in the chosen sequence is changed to a different amino acid, provided that no more than 15% of the amino acid residues in the sequence are so changed; (e) a nucleic acid fragment encoding at least a portion of a polypeptide comprising the amino acid sequence selected from the group consisting of SEQ ID NO:2n, wherein n is an integer between 1 and 141, or any variant of said polypeptide wherein any amino acid of the chosen sequence is changed to a different amino acid, provided that no more than 10% of the amino acid residues in the sequence are so changed; and (f) the complement of any of said nucleic acid molecules.

In yet another specific embodiment, the invention includes an isolated nucleic acid molecule, wherein said nucleic acid molecule comprises a nucleotide sequence selected from the group consisting of: (a) the nucleotide sequence selected from the group consisting of SEQ ID NO:2n−1, wherein n is an integer between 1 and 141; (b) a nucleotide sequence wherein one or more nucleotides in the nucleotide sequence selected from the group consisting of SEQ ID NO:2n−1, wherein n is an integer between 1 and 141, is changed from that selected from the group consisting of the chosen sequence to a different nucleotide provided that no more than 15% of the nucleotides are so changed; (c) a nucleic acid fragment of the sequence selected from the group consisting of SEQ ID NO:2n−1, wherein n is an integer between 1 and 141; and (d) a nucleic acid fragment wherein one or more nucleotides in the nucleotide sequence selected from the group consisting of SEQ ID NO:2n−1, wherein n is an integer between 1 and 141, is changed from that selected from the group consisting of the chosen sequence to a different nucleotide provided that no more than 15% of the nucleotides are so changed.

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

A NOVX nucleic acid can encode a mature NOVX polypeptide. As used herein, a “mature” form of a polypeptide or protein disclosed in the present invention is the product of a naturally occurring polypeptide, precursor form, or proprotein. The naturally occurring polypeptide, precursor or proprotein includes, by way of nonlimiting example, the full-length gene product encoded by the corresponding gene. Alternatively, it may be defined as the polypeptide, precursor or proprotein encoded by an ORF described herein. The product “mature” form arises, by way of nonlimiting example, as a result of one or more naturally occurring processing steps that may take place within the cell (e.g., host cell) in which the gene product arises. Examples of such processing steps leading to a “mature” form of a polypeptide or protein include the cleavage of the N-terminal methionine residue encoded by the initiation codon of an ORF or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a post-translational modification step other than a proteolytic cleavage event. Such additional processes include, by way of non-limiting example, glycosylation, myristylation or phosphorylation. In general, a mature polypeptide or protein may result from the operation of only one of these processes, or a combination of any of them.

The term “probe”, as utilized herein, refers to nucleic acid sequences of variable length, preferably between at least about 10 nucleotides (nt), about 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as used herein, is a nucleic acid that is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, about 4 kb, about 3 kb, about 2 kb, about 1 kb, about 0.5 kb, or about 0.1 kb, of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material, or culture medium, or of chemical precursors or other chemicals.

A nucleic acid molecule of the invention, e.g., a nucleic acid molecule having the nucleotide sequence of SEQ ID NOS: 2n−1, wherein n is an integer between 1 and 141, or a complement of this nucleotide sequence, can be isolated using standard molecular biology techniques and the sequence information provided herein. Using all or a portion of the nucleic acid sequence of SEQ ID NOS:2n−1, wherein n is an integer between 1 and 141, as a hybridization probe, NOVX molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993).

A nucleic acid of the invention can be amplified using cDNA, mRNA or, alternatively, genomic DNA as a template with appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides of SEQ ID NOS:2n−1, wherein n is an integer between 1 and 141, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NOS:2n−1, wherein n is an integer between 1 and 141, or a portion of this nucleotide sequence (e.g., a fragment that can be used as a probe or primer or a fragment encoding a biologically-active portion of a NOVX polypeptide). A nucleic acid molecule that is complementary to the nucleotide sequence of SEQ ID NOS:2n−1, wherein n is an integer between 1 and 141, is one that is sufficiently complementary to the nucleotide sequence of SEQ ID NOS:2n−1, wherein n is an integer between 1 and 141, that it can hydrogen bond with few or no mismatches to a nucleotide sequence of SEQ ID NOS:2n−1, wherein n is an integer between 1 and 141, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

A “fragment” provided herein is defined as a sequence of at least 6 (contiguous) nucleic acids or at least 4 (contiguous) amino acids, a length sufficient to allow for specific hybridization in the case of nucleic acids or for specific recognition of an epitope in the case of amino acids, and is at most some portion less than a full length sequence. Fragments may be derived from any contiguous portion of a nucleic acid or amino acid sequence of choice.

A full-length NOVX clone is identified as containing an ATG translation start codon and an in-frame stop codon. Any disclosed NOVX nucleotide sequence lacking an ATG start codon therefore encodes a truncated C-terminal fragment of the respective NOVX polypeptide, and requires that the corresponding full-length cDNA extend in the 5′ direction of the disclosed sequence. Any disclosed NOVX nucleotide sequence lacking an in-frame stop codon similarly encodes a truncated N-terminal fragment of the respective NOVX polypeptide, and requires that the corresponding full-length cDNA extend in the 3′ direction of the disclosed sequence.

A “derivative” is a nucleic acid sequence or amino acid sequence formed from the native compounds either directly, by modification or partial substitution. An “analog” is a nucleic acid sequence or amino acid sequence that has a structure similar to, but not identical to, the native compound, e.g. they differs from it in respect to certain components or side chains. Analogs may be synthetic or derived from a different evolutionary origin and may have a similar or opposite metabolic activity compared to wild type. A “homolog” is a nucleic acid sequence or amino acid sequence of a particular gene that is derived from different species.

Derivatives and analogs may be full length or other than full length. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences include those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for a NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NO:2n−1, wherein n is an integer between 1 and 141, as well as a polypeptide possessing NOVX biological activity. Various biological activities of the NOVX proteins are described below.

A NOVX polypeptide is encoded by the open reading frame (“ORF”) of a NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bonafide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g. from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of SEQ ID NO:2n−1, wherein n is an integer between 1 and 141; or an anti-sense strand nucleotide sequence of SEQ ID NO:2n−1, wherein n is an integer between 1 and 141; or of a naturally occurring mutant of SEQ ID NO:2n−1, wherein n is an integer between 1 and 141.

Probes based on the human NOVX nucleotide sequences can be used to detect transcripts or genomic sequences encoding the same or homologous proteins. In various embodiments, the probe has a detectable label attached, e.g. the label can be a radioisotope, a fluorescent compound, an enzyme, or an enzyme co-factor. Such probes can be used as a part of a diagnostic test kit for identifying cells or tissues which mis-express a NOVX protein, such as by measuring a level of a NOVX-encoding nucleic acid in a sample of cells from a subject e.g., detecting NOVX mRNA levels or determining whether a genomic NOVX gene has been mutated or deleted.

“A polypeptide having a biologically-active portion of a NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion of SEQ ID NO:2n−1, wherein n is an integer between 1 and 141, that encodes a polypeptide having a NOVX biological activity (the biological activities of the NOVX proteins are described below), expressing the encoded portion of NOVX protein (e.g., by recombinant expression in vitro) and assessing the activity of the encoded portion of NOVX.

NOVX Nucleic Acid and Polypeptide Variants

The invention further encompasses nucleic acid molecules that differ from the nucleotide sequences of SEQ ID NO:2n−1, wherein n is an integer between 1 and 141, due to degeneracy of the genetic code and thus encode the same NOVX proteins as that encoded by the nucleotide sequences of SEQ ID NO:2n−1, wherein n is an integer between 1 and 141. In another embodiment, an isolated nucleic acid molecule of the invention has a nucleotide sequence encoding a protein having an amino acid sequence of SEQ ID NO:2n, wherein n is an integer between 1 and 141.

In addition to the human NOVX nucleotide sequences of SEQ ID NO:2n−1, wherein n is an integer between 1 and 141, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding a NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from a human SEQ ID NO:2n−1, wherein n is an integer between 1 and 141, are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NO:2n−1, wherein n is an integer between 1 and 141. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least about 65% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to a sequence of SEQ ID NO:2n−1, wherein n is an integer between 1 and 141, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NO:2n−1, wherein n is an integer between 1 and 141, or fragments, analogs or derivatives thereof, under conditions of moderate stringency is provided. A non-limiting example of moderate stringency hybridization conditions are hybridization in 6× SSC, 5×Reinhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, NY, and Krieger, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences of SEQ ID NO:2n−1, wherein n is an integer between 1 and 141, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

Conservative Mutations

In addition to naturally-occurring allelic variants of NOVX sequences that may exist in the population, the skilled artisan will further appreciate that changes can be introduced by mutation into the nucleotide sequences of SEQ ID NO:2n−1, wherein n is an integer between 1 and 141, thereby leading to changes in the amino acid sequences of the encoded NOVX protein, without altering the functional ability of that NOVX protein. For example, nucleotide substitutions leading to amino acid substitutions at “non-essential” amino acid residues can be made in the sequence of SEQ ID NO:2n, wherein n is an integer between 1 and 141. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are predicted to be particularly non-amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NO:2n−1, wherein n is an integer between 1 and 141, yet retain biological activity. In one embodiment, the isolated nucleic acid molecule comprises a nucleotide sequence encoding a protein, wherein the protein comprises an amino acid sequence at least about 40% homologous to the amino acid sequences of SEQ ID NO:2n, wherein n is an integer between 1 and 141. Preferably, the protein encoded by the nucleic acid molecule is at least about 60% homologous to SEQ ID NO:2n, wherein n is an integer between 1 and 141; more preferably at least about 70% homologous to SEQ ID NO:2n, wherein n is an integer between 1 and 141; still more preferably at least about 80% homologous to SEQ ID NO:2n, wherein n is an integer between 1 and 141; even more preferably at least about 90% homologous to SEQ ID NO:2n, wherein n is an integer between 1 and 141; and most preferably at least about 95% homologous to SEQ ID NO:2n, wherein n is an integer between 1 and 141.

An isolated nucleic acid molecule encoding a NOVX protein homologous to the protein of SEQ ID NO:2n, wherein n is an integer between 1 and 141, can be created by introducing one or more nucleotide substitutions, additions or deletions into the nucleotide sequence of SEQ ID NO:2n−1, wherein n is an integer between 1 and 141, such that one or more amino acid substitutions, additions or deletions are introduced into the encoded protein.

Mutations can be introduced any one of SEQ ID NO:2n−1, wherein n is an integer between 1 and 141, by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the NOVX protein is replaced with another amino acid residue from the same side chain family. Alternatively, in another embodiment, mutations can be introduced randomly along all or part of a NOVX coding sequence, such as by saturation mutagenesis, and the resultant mutants can be screened for NOVX biological activity to identify mutants that retain activity. Following mutagenesis of a nucleic acid of SEQ ID NO:2n−1, wherein n is an integer between 1 and 141, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and a NOVX ligand; or (iii) the ability of a mutant NOVX protein to bind to an intracellular target protein or biologically-active portion thereof; (e.g. avidin proteins).

In yet another embodiment, a mutant NOVX protein can be assayed for the ability to regulate a specific biological function (e.g., regulation of insulin release).

Interfering RNA

In one aspect of the invention, NOVX gene expression can be attenuated by RNA interference. One approach well-known in the art is short interfering RNA (siRNA) mediated gene silencing where expression products of a NOVX gene are targeted by specific double stranded NOVX derived siRNA nucleotide sequences that are complementary to at least a 19-25 nt long segment of the NOVX gene transcript, including the 5′ untranslated (UT) region, the ORF, or the 3′ UT region. See, e.g., PCT applications WO00/44895, WO99/32619, WO01/75164, WO01/92513, WO01/29058, WO01/89304, WO02/16620, and WO02/29858, each incorporated by reference herein in their entirety. Targeted genes can be a NOVX gene, or an upstream or downstream modulator of the NOVX gene. Nonlimiting examples of upstream or downstream modulators of a NOVX gene include, e.g., a transcription factor that binds the NOVX gene promoter, a kinase or phosphatase that interacts with a NOVX polypeptide, and polypeptides involved in a NOVX regulatory pathway.

According to the methods of the present invention, NOVX gene expression is silenced using short interfering RNA. A NOVX polynucleotide according to the invention includes a siRNA polynucleotide. Such a NOVX siRNA can be obtained using a NOVX polynucleotide sequence, for example, by processing the NOVX ribopolynucleotide sequence in a cell-free system, such as but not limited to a Drosophila extract, or by transcription of recombinant double stranded NOVX RNA or by chemical synthesis of nucleotide sequences homologous to a NOVX sequence. See, e.g., Tuscbl, Zamore, Lehmann, Bartel and Sharp (1999), Genes & Dev. 13: 3191-3197, incorporated herein by reference in its entirety. When synthesized, a typical 0.2 micromolar-scale RNA synthesis provides about 1 milligram of siRNA, which is sufficient for 1000 transfection experiments using a 24-well tissue culture plate format.

The most efficient silencing is generally observed with siRNA duplexes composed of a 21-nt sense strand and a 21-nt antisense strand, paired in a manner to have a 2-nt 3′ overhang. The sequence of the 2-nt 3′ overhang makes an additional small contribution to the specificity of siRNA target recognition. The contribution to specificity is localized to the unpaired nucleotide adjacent to the first paired bases. In one embodiment, the nucleotides in the 3′ overhang are ribonucleotides. In an alternative embodiment, the nucleotides in the 3′ overhang are deoxyribonucleotides. Using 2′-deoxyribonucleotides in the 3′ overhangs is as efficient as using ribonucleotides, but deoxyribonucleotides are often cheaper to synthesize and are most likely more nuclease resistant.

A contemplated recombinant expression vector of the invention comprises a NOVX DNA molecule cloned into an expression vector comprising operatively-linked regulatory sequences flanking the NOVX sequence in a manner that allows for expression (by transcription of the DNA molecule) of both strands. An RNA molecule that is antisense to NOVX mRNA is transcribed by a first promoter (e.g., a promoter sequence 3′ of the cloned DNA) and an RNA molecule that is the sense strand for the NOVX mRNA is transcribed by a second promoter (e.g., a promoter sequence 5′ of the cloned DNA). The sense and antisense strands may hybridize in vivo to generate siRNA constructs for silencing of the NOVX gene. Alternatively, two constructs can be utilized to create the sense and anti-sense strands of a siRNA construct. Finally, cloned DNA can encode a construct having secondary structure, wherein a single transcript has both the sense and complementary antisense sequences from the target gene or genes. In an example of this embodiment, a hairpin RNAi product is homologous to all or a portion of the target gene. In another example, a hairpin RNAi product is a siRNA. The regulatory sequences flanking the NOVX sequence may be identical or may be different, such that their. expression may be modulated independently, or in a temporal or spatial manner.

In a specific embodiment, siRNAs are transcribed intracellularly by cloning the NOVX gene templates into a vector containing, e.g., a RNA pol III transcription unit from the smaller nuclear RNA (snRNA) U6 or the human RNase P RNA H1. One example of a vector system is the GeneSuppressor™ RNA Interference kit (commercially available from Imgenex). The U6 and H1 promoters are members of the type III class of Pol III promoters. The +1 nucleotide of the U6-like promoters is always guanosine, whereas the +1 for H1 promoters is adenosine. The termination signal for these promoters is defined by five consecutive thymidines. The transcript is typically cleaved after the second uridine. Cleavage at this position generates a 3′ UU overhang in the expressed siRNA, which is similar to the 3′ overhangs of synthetic siRNAs. Any sequence less than 400 nucleotides in length can be transcribed by these promoter, therefore they are ideally suited for the expression of around 21-nucleotide siRNAs in, e.g., an approximately 50-nucleotide RNA stem-loop transcript.

A siRNA vector appears to have an advantage over synthetic siRNAs where long term knock-down of expression is desired. Cells transfected with a siRNA expression vector would experience steady, long-term mRNA inhibition. In contrast, cells transfected with exogenous synthetic siRNAs typically recover from mRNA suppression within seven days or ten rounds of cell division. The long-term gene silencing ability of siRNA expression vectors may provide for applications in gene therapy.

In general, siRNAs are chopped from longer dsRNA by an ATP-dependent ribonuclease called DICER. DICER is a member of the RNase III family of double-stranded RNA-specific endonucleases. The siRNAs assemble with cellular proteins into an endonuclease complex. In vitro studies in Drosophila suggest that the siRNAs/protein complex (siRNP) is then transferred to a second enzyme complex, called an RNA-induced silencing complex (RISC), which contains an endoribonuclease that is distinct from DICER. RISC uses the sequence encoded by the antisense siRNA strand to find and destroy mRNAs of complementary sequence. The siRNA thus acts as a guide, restricting the ribonuclease to cleave only mRNAs complementary to one of the two siRNA strands.

A NOVX mRNA region to be targeted by siRNA is generally selected from a desired NOVX sequence beginning 50 to 100 nt downstream of the start codon. Alternatively, 5′ or 3′ UTRs and regions nearby the start codon can be used but are generally avoided, as these may be richer in regulatory protein binding sites. UTR-binding proteins and/or translation initiation complexes may interfere with binding of the siRNP or RISC endonuclease complex. An initial BLAST homology search for the selected siRNA sequence is done against an available nucleotide sequence library to ensure that only one gene is targeted. Specificity of target recognition by siRNA duplexes indicate that a single point mutation located in the paired region of an siRNA duplex is sufficient to abolish target mRNA degradation. See, Elbashir et al. 2001 EMBO J. 20(23):6877-88. Hence, consideration should be taken to accommodate SNPs, polymorphisms, allelic variants or species-specific variations when targeting a desired gene.

In one embodiment, a complete NOVX siRNA experiment includes the proper negative control. A negative control siRNA generally has the same nucleotide composition as the NOVX siRNA but lack significant sequence homology to the genome. Typically, one would scramble the nucleotide sequence of the NOVX siRNA and do a homology search to make sure it lacks homology to any other gene.

Two independent NOVX siRNA duplexes can be used to knock-down a target NOVX gene. This helps to control for specificity of the silencing effect. In addition, expression of two independent genes can be simultaneously knocked down by using equal concentrations of different NOVX siRNA duplexes, e.g., a NOVX siRNA and an siRNA for a regulator of a NOVX gene or polypeptide. Availability of siRNA-associating proteins is believed to be more limiting than target mRNA accessibility.

A targeted NOVX region is typically a sequence of two adenines (AA) and two thymidines (TT) divided by a spacer region of nineteen (N 19) residues (e.g., AA(N19)TT). A desirable spacer region has a G/C-content of approximately 30% to 70%, and more preferably of about 50%. If the sequence AA(N19)TT is not present in the target sequence, an alternative target region would be AA(N21). The sequence of the NOVX sense siRNA corresponds to (N19)TT or N21, respectively. In the latter case, conversion of the 3′ end of the sense siRNA to TT can be performed if such a sequence does not naturally occur in the NOVX polynucleotide. The rationale for this sequence conversion is to generate a symmetric duplex with respect to the sequence composition of the sense and antisense 3′ overhangs. Symmetric 3′ overhangs may help to ensure that the siRNPs are formed with approximately equal ratios of sense and antisense target RNA-cleaving siRNPs. See, e.g., Elbashir, Lendeckel and Tuschl (2001). Genes & Dev. 15: 188-200, incorporated by reference herein in its entirely. The modification of the overhang of the sense sequence of the siRNA duplex is not expected to affect targeted mRNA recognition, as the antisense siRNA strand guides target recognition.

Alternatively, if the NOVX target mRNA does not contain a suitable AA(N21) sequence, one may search for the sequence NA(N21). Further, the sequence of the sense strand and antisense strand may still be synthesized as 5′ (N19)TT, as it is believed that the sequence of the 3′-most nucleotide of the antisense siRNA does not contribute to specificity. Unlike antisense or ribozyme technology, the secondary structure of the target mRNA does not appear to have a strong effect on silencing. See, Harborth, et al. (2001) J. Cell Science 114: 4557-4565, incorporated by reference in its entirety.

Transfection of NOVX siRNA duplexes can be achieved using standard nucleic acid transfection methods, for example, OLIGOFECTAMINE Reagent (commercially available from Invitrogen). An assay for NOVX gene silencing is generally performed approximately 2 days after transfection. No NOVX gene silencing has been observed in the absence of transfection reagent, allowing for a comparative analysis of the wild-type and silenced NOVX phenotypes. In a specific embodiment, for one well of a 24-well plate, approximately 0.84 μg of the siRNA duplex is generally sufficient. Cells are typically seeded the previous day, and are transfected at about 50% confluence. The choice of cell culture media and conditions are routine to those of skill in the art, and will vary with the choice of cell type. The efficiency of transfection may depend on the cell type, but also on the passage number and the confluency of the cells. The time and the manner of formation of siRNA-liposome complexes (e.g. inversion versus vortexing) are also critical. Low transfection efficiencies are the most frequent cause of unsuccessful NOVX silencing. The efficiency of transfection needs to be carefully examined for each new cell line to be used. Preferred cell are derived from a mammal, more preferably from a rodent such as a rat or mouse, and most preferably from a human. Where used for therapeutic treatment, the cells are preferentially autologous, although non-autologous cell sources are also contemplated as within the scope of the present invention.

For a control experiment, transfection of 0.84 μg single-stranded sense NOVX siRNA will have no effect on NOVX silencing, and 0.84 μg antisense siRNA has a weak silencing effect when compared to 0.84 μg of duplex siRNAs. Control experiments again allow for a comparative analysis of the wild-type and silenced NOVX phenotypes. To control for transfection efficiency, targeting of common proteins is typically performed, for example targeting of lamin A/C or transfection of a CMV-driven EGFP-expression plasmid (e.g. commercially available from Clontech). In the above example, a determination of the fraction of lamin A/C knockdown in cells is determined the next day by such techniques as immunofluorescence, Western blot, Northern blot or other similar assays for protein expression or gene expression. Lamin A/C monoclonal antibodies may be obtained from Santa Cruz Biotechnology.

Depending on the abundance and the half life (or turnover) of the targeted NOVX polynucleotide in a cell, a knock-down phenotype may become apparent after 1 to 3 days, or even later. In cases where no NOVX knock-down phenotype is observed, depletion of the NOVX polynucleotide may be observed by immunofluorescence or Western blotting. If the NOVX polynucleotide is still abundant after 3 days, cells need to be split and transferred to a fresh 24-well plate for re-transfection. If no knock-down of the targeted protein is observed, it may be desirable to analyze whether the target mRNA (NOVX or a NOVX upstream or downstream gene) was effectively destroyed by the transfected siRNA duplex. Two days after transfection, total RNA is prepared, reverse transcribed using a target-specific primer, and PCR-amplified with a primer pair covering at least one exon-exon junction in order to control for amplification of pre-mRNAs. RT/PCR of a non-targeted mRNA is also needed as control. Effective depletion of the mRNA yet undetectable reduction of target protein may indicate that a large reservoir of stable NOVX protein may exist in the cell. Multiple transfection in sufficiently long intervals may be necessary until the target protein is finally depleted to a point where a phenotype may become apparent. If multiple transfection steps are required, cells are split 2 to 3 days after transfection. The cells may be transfected immediately after splitting.

An inventive therapeutic method of the invention contemplates administering a NOVX siRNA construct as therapy to compensate for increased or aberrant NOVX expression or activity. The NOVX ribopolynucleotide is obtained and processed into siRNA fragments, or a NOVX siRNA is synthesized, as described above. The NOVX siRNA is administered to cells or tissues using known nucleic acid transfection techniques, as described above. A NOVX siRNA specific for a NOVX gene will decrease or knockdown NOVX transcription products, which will lead to reduced NOVX polypeptide production, resulting in reduced NOVX polypeptide activity in the cells or tissues.

The present invention also encompasses a method of treating a disease or condition associated with the presence of a NOVX protein in an individual comprising administering to the individual an RNAi construct that targets the mRNA of the protein (the mRNA that encodes the protein) for degradation. A specific RNAi construct includes a siRNA or a double stranded gene transcript that is processed into siRNAs. Upon treatment, the target protein is not produced or is not produced to the extent it would be in the absence of the treatment.

Where the NOVX gene function is not correlated with a known phenotype, a control sample of cells or tissues from healthy individuals provides a reference standard for determining NOVX expression levels. Expression levels are detected using the assays described, e.g., RT-PCR, Northern blotting, Western blotting, ELISA, and the like. A subject sample of cells or tissues is taken from a mammal, preferably a human subject, suffering from a disease state. The NOVX ribopolynucleotide is used to produce siRNA constructs, that are specific for the NOVX gene product. These cells or tissues are treated by administering NOVX siRNA's to the cells or tissues by methods described for the transfection of nucleic acids into a cell or tissue, and a change in NOVX polypeptide or polynucleotide expression is observed in the subject sample relative to the control sample, using the assays described. This NOVX gene knockdown approach provides a rapid method for determination of a NOVX minus (NOVX⁻) phenotype in the treated subject sample. The NOVX⁻ phenotype observed in the treated subject sample thus serves as a marker for monitoring the course of a disease state during treatment.

In specific embodiments, a NOVX siRNA is used in therapy. Methods for the generation and use of a NOVX siRNA are known to those skilled in the art. Example techniques are provided below.

Production of RNAs

Sense RNA (ssRNA) and antisense RNA (asRNA) of NOVX are produced using known methods such as transcription in RNA expression vectors. In the initial experiments, the sense and antisense RNA are about 500 bases in length each. The produced ssRNA and asRNA (0.5 μM) in 10 mM Tris-HCl (pH 7.5) with 20 mM NaCl were heated to 95° C. for 1 min then cooled and annealed at room temperature for 12 to 16 h. The RNAs are precipitated and resuspended in lysis buffer (below). To monitor annealing, RNAs are electrophoresed in a 2% agarose gel in TBE buffer and stained with ethidium bromide. See, e.g., Sambrook et al., Molecular Cloning. Cold Spring Harbor Laboratory Press, Plainview, N.Y. (1989).

Lysate Preparation

Untreated rabbit reticulocyte lysate (Ambion) are assembled according to the manufacturer's directions. dsRNA is incubated in the lysate at 30° C. for 10 min prior to the addition of mRNAs. Then NOVX mRNAs are added and the incubation continued for an additional 60 min. The molar ratio of double stranded RNA and mRNA is about 200:1. The NOVX mRNA is radiolabeled (using known techniques) and its stability is monitored by gel electrophoresis.

In a parallel experiment made with the same conditions, the double stranded RNA is internally radiolabeled with a ³²P-ATP. Reactions are stopped by the addition of 2× proteinase K buffer and deproteinized as described previously (Tuschl et al., Genes Dev., 13:3191-3197 (1999)). Products are analyzed by electrophoresis in 15% or 18% polyacrylamide sequencing gels using appropriate RNA standards. By monitoring the gels for radioactivity, the natural production of 10 to 25 nt RNAs from the double stranded RNA can be determined.

The band of double stranded RNA, about 21-23 bps, is eluded. The efficacy of these 21-23 mers for suppressing NOVX transcription is assayed in vitro using the same rabbit reticulocyte assay described above using 50 nanomolar of double stranded 21-23 mer for each assay. The sequence of these 21-23 mers is then determined using standard nucleic acid sequencing techniques.

RNA Preparation

21 nt RNAs, based on the sequence determined above, are chemically synthesized using Expedite RNA phosphoramidites and thymidine phosphoramidite (Proligo, Germany). Synthetic oligonucleotides are deprotected and gel-purified (Elbashir, Lendeckel, & Tuschl, Genes & Dev. 15, 188-200 (2001)), followed by Sep-Pak C18 cartridge (Waters, Milford, Mass., USA) purification (Tuschl, et al., Biochemistry, 32:11658-11668 (1993)).

These RNAs (20 μM) single strands are incubated in annealing buffer (100 mM potassium acetate, 30 mM HEPES-KOH at pH 7.4, 2 mM magnesium acetate) for 1 min at 90° C. followed by 1 h at 37° C.

Cell Culture

A cell culture known in the art to regularly express NOVX is propagated using standard conditions. 24 hours before transfection, at approx. 80% confluency, the cells are trypsinized and diluted 1:5 with fresh medium without antibiotics (1-3×105 cells/ml) and transferred to 24-well plates (500 ml/well). Transfection is performed using a commercially available lipofection kit and NOVX expression is monitored using standard techniques with positive and negative control. A positive control is cells that naturally express NOVX while a negative control is cells that do not express NOVX. Base-paired 21 and 22 nt siRNAs with overhanging 3′ ends mediate efficient sequence-specific mRNA degradation in lysates and in cell culture. Different concentrations of siRNAs are used. An efficient concentration for suppression in vitro in mammalian culture is between 25 nM to 100 nM final concentration. This indicates that siRNAs are effective at concentrations that are several orders of magnitude below the concentrations applied in conventional antisense or ribozyme gene targeting experiments.

The above method provides a way both for the deduction of NOVX siRNA sequence and the use of such siRNA for in vitro suppression. In vivo suppression may be performed using the same siRNA using well known in vivo transfection or gene therapy transfection techniques.

Antisense Nucleic Acids

Another aspect of the invention pertains to isolated antisense nucleic acid molecules that are hybridizable to or complementary to the nucleic acid molecule comprising the nucleotide sequence of SEQ ID NO:2n−1, wherein n is an integer between 1 and 141, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of a NOVX protein of SEQ ID NO:2n, wherein n is an integer between 1 and 141, or antisense nucleic acids complementary to a NOVX nucleic acid sequence of SEQ ID NO:2n−1, wherein n is an integer between 1 and 141, are additionally provided.

In one embodiment, an antisense nucleic acid molecule is antisense to a “coding region” of the coding strand of a nucleotide sequence encoding a NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences which flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-carboxymethylaminomethyl-2-thiouridine, 5-(carboxyhydroxylmethyl) uracil, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methyl guanine, 5-methoxyuracil, 3-methylcytosine, 5-methylcytosine, N6-adenine, 7-methylguanine, 5-methylaminomethyluracil, 5-methoxyaminomethyl-2-thiouracil, 2-thiouracil, 4-thiouracil, beta-D-mannosylqueosine, 5′-methoxycarboxymethyluracil, 2-methylthio-N6-isopentenyladenine, uracil-5-oxyacetic acid (v), wybutoxosine, pseudouracil, queosine, 2-thiocytosine, 5-methyl-2-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (See, e.g., Inoue, et al. 1987. Nucl. Acids Res. 15: 6131-6148) or a chimeric RNA-DNA analogue (See, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for a NOVX-encoding nucleic acid can be designed based upon the nucleotide sequence of a NOVX cDNA disclosed herein (i.e., SEQ ID NO:2n−1, wherein n is an integer between 1 and 141). For example, a derivative of a Tetrahymena L-19 IVS RNA can be constructed in which the nucleotide sequence of the active site is complementary to the nucleotide sequence to be cleaved in a NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann. N.Y Acad. Sci. 660: 27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleotide bases are retained. The neutral backbone of PNAs has been shown to allow for specific hybridization to DNA and RNA under conditions of low ionic strength. The synthesis of PNA oligomer can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also bemused, for example, in the analysis of single base pair mutations in a gene (e.g., PNA directed PCR clamping; as artificial restriction enzymes when used in combination with other enzymes, e.g., S₁ nucleases (See, Hyrup, et al., 1996.supra); or as probes or primers for DNA sequence and hybridization (See, Hyrup, et al., 1996, supra; Perry-O'Keefe, et al., 1996. supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleotide bases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in any one of SEQ ID NO:2n, wherein n is an integer between 1 and 141. The invention also includes a mutant or variant protein any of whose residues may be changed from the corresponding residues shown in any one of SEQ ID NO:2n, wherein n is an integer between 1 and 141, while still encoding a protein that maintains its NOVX activities and physiological functions, or a functional fragment thereof.

In general, a NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, a NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence of SEQ ID NO:2n, wherein n is an integer between 1 and 141) that include fewer amino acids than the full-length NOVX proteins, and exhibit at least one activity of a NOVX protein. Typically, biologically-active portions comprise a domain or motif with at least one activity of the NOVX protein. A biologically-active portion of a NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence of SEQ ID NO:2n, wherein n is an integer between 1 and 141. In other embodiments, the NOVX protein is substantially homologous to SEQ ID NO:2n, wherein n is an integer between 1 and 141, and retains the functional activity of the protein of SEQ ID NO:2n, wherein n is an integer between 1 and 141, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence of SEQ ID NO:2n, wherein n is an integer between 1 and 141, and retains the functional activity of the NOVX proteins of SEQ ID NO:2n, wherein n is an integer between 1 and 141.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence of SEQ ID NO:2n−1, wherein n is an integer between 1 and 141.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, a NOVX “chimeric protein” or “fusion protein” comprises a NOVX polypeptide operatively-linked to a non-NOVX polypeptide. An “NOVX polypeptide” refers to a polypeptide having an amino acid sequence corresponding to a NOVX protein of SEQ ID NO:2n, wherein n is an integer between 1 and 141, whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within a NOVX fusion protein the NOVX polypeptide can correspond to all or a portion of a NOVX protein. In one embodiment, a NOVX fusion protein comprises at least one biologically-active portion of a NOVX protein. In another embodiment, a NOVX fusion protein comprises at least two biologically-active portions of a NOVX protein. In yet another embodiment, a NOVX fusion protein comprises at least three biologically-active portions of a NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is a NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is a NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between a NOVX ligand and a NOVX protein on the surface of a cell, to thereby suppress NOVX-mediated signal transduction in vivo. The NOVX-immunoglobulin fusion proteins can be used to affect the bioavailability of a NOVX cognate ligand. Inhibition of the NOVX ligand/NOVX interaction may be useful therapeutically for both the treatment of proliferative and differentiative disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with a NOVX ligand.

A NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the NOVX protein coding sequences can be used to generate a variegated population of NOVX fragments for screening and subsequent selection of variants of a NOVX protein. In one embodiment, a library of coding sequence fragments can be generated by treating a double stranded PCR fragment of a NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad. Sci. USA 89: 7811-7815; Delgrave, et al., 1993. Protein Engineering 6:327-331.

NOVX Antibodies

The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an F_{ab} expression library. In general, antibody molecules obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated protein of the invention intended to serve as an antigen, or a portion or fragment thereof, can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein, such as an amino acid sequence of SEQ ID NO:2n, wherein n is an integer between 1 and 141, and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of NOVX that is located on the surface of the protein, e.g., a hydrophilic region. A hydrophobicity analysis of the human NOVX protein sequence will indicate which regions of a NOVX polypeptide are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each incorporated herein by reference in their entirety. Antibodies that are specific for one or more domains within an antigenic protein, or derivatives, fragments, analogs or homologs thereof, are also provided herein.

The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. A NOVX polypeptide or a fragment thereof comprises at least one antigenic epitope. An anti-NOVX antibody of the present invention is said to specifically bind to antigen NOVX when the equilibrium binding constant (K_{D}) is ≦1 μM, preferably ≦100 nM, more preferably ≦10 nM, and most preferably ≦100 pM to about 1 pM, as measured by assays such as radioligand binding assays or similar assays known to those skilled in the art.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp.51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). It is an objective, especially important in therapeutic applications of monoclonal antibodies, to identify antibodies having a high degree of specificity and a high binding affinity for the target antigen.

After the desired hybridoma cells are identified, the clones can be subcloned by limiting dilution procedures and grown by standard methods (Goding, 1986). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fe), typically that of a human immunoglobulin (Jones et al., 1986; Riechmann et al., 1988; and Presta, Curr. Op. Struct. Biol., 2:593-596 (1992)).

Human Antibodies

Fully human antibodies essentially relate to antibody molecules in which the entire sequence of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hodgenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368 812-13 (1994)); Fishwild et al,(Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridylditbiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

Immunoliposomes

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide-interchange reaction. A chemotherapeutic agent (such as Doxorubicin) is optionally contained within the liposome. See Gabizon et al., J. National Cancer Inst., 81(19): 1484 (1989).

Diagnostic Applications of Antibodies Directed Against the Proteins of the Invention

Antibodies directed against a protein of the invention may be used in methods known within the art relating to the localization and/or quantitation of the protein (e.g., for use in measuring levels of the protein within appropriate physiological samples, for use in diagnostic methods, for use in imaging the protein, and the like). In a given embodiment, antibodies against the proteins, or derivatives, fragments, analogs or homologs thereof, that contain the antigen binding domain, are utilized as pharmacologically-active compounds (see below).

An antibody specific for a protein of the invention can be used to isolate the protein by standard techniques, such as immunoaffinity chromatography or immunoprecipitation. Such an antibody can facilitate the purification of the natural protein antigen from cells and of recombinantly produced antigen expressed in host cells. Moreover, such an antibody can be used to detect the antigenic protein (e.g., in a cellular lysate or cell supernatant) in order to evaluate the abundance and pattern of expression of the antigenic protein. Antibodies directed against the protein can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidinibiotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibody Therapeutics

Antibodies of the invention, including polyclonal, monoclonal, humanized and fully human antibodies, may used as therapeutic agents. Such agents will generally be employed to treat or prevent a disease or pathology in a subject. An antibody preparation, preferably one having high specificity and high affinity for its target antigen, is administered to the subject and will generally have an effect due to its binding with the target. Such an effect may be one of two kinds, depending on the specific nature of the interaction between the given antibody molecule and the target antigen in question. In the first instance, administration of the antibody may abrogate or inhibit the binding of the target with an endogenous ligand to which it naturally binds. In this case, the antibody binds to the target and masks a binding site of the naturally occurring ligand, wherein the ligand serves as an effector molecule. Thus the receptor mediates a signal transduction pathway for which ligand is responsible.

Alternatively, the effect may be one in which the antibody elicits a physiological result by virtue of binding to an effector binding site on the target molecule. In this case the target, a receptor having an endogenous ligand which may be absent or defective in the disease or pathology, binds the antibody as a surrogate effector ligand, initiating a receptor-based signal transduction event by the receptor.

A therapeutically effective amount of an antibody of the invention relates generally to the amount needed to achieve a therapeutic objective. As noted above, this may be a binding interaction between the antibody and its target antigen that, in certain cases, interferes with the functioning of the target, and in other cases, promotes a physiological response. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or, antibody fragment of the invention may be, by way of nonlimiting example, from about 0.1 mg/kg body weight to about 50 mg/kg body weight. Common dosing frequencies may range, for example, from twice daily to once a week.

Pharmaceutical Compositions of Antibodies

Antibodies specifically binding a protein of the invention, as well as other molecules identified by the screening assays disclosed herein, can be administered for the treatment of various disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

If the antigenic protein is intracellular and whole antibodies are used as inhibitors, internalizing antibodies are preferred. However, liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., Proc. Natl. Acad. Sci. USA, 90: 7889-7893 (1993). The formulation herein can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

ELISA Assay

An agent for detecting an analyte protein is an antibody capable of binding to an analyte protein, preferably an antibody with a detectable label. Antibodies can be polyclonal, or more preferably, monoclonal. An intact antibody, or a fragment thereof (e.g., F_{ab} or F_{(ab)2}) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the invention can be used to detect an analyte mRNA, protein, or genomic DNA in a biological sample in vitro as well as in vivo. For example, in vitro techniques for detection of an analyte mRNA include Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, N.J., 1995; “Immunoassay”, E. Diamandis and T. Christopoulus, Academic Press, Inc., San Diego, Calif., 1996; and “Practice and Theory of Enzyme Immunoassays”, P. Tijssen, Elsevier Science Publishers, Amsterdam, 1985. Furthermore, in vivo techniques for detection of an analyte protein include introducing into a subject a labeled anti-an analyte protein antibody. For example, the antibody can be labeled with a radioactive marker whose presence and location in a subject can be detected by standard imaging techniques.

NOVX Recombinant Expression Vectors and Host Cells

Another aspect of the invention pertains to vectors, preferably expression vectors, containing a nucleic acid encoding a NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, useful expression vectors in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec 1 (Baldari, et al., 1987. EMBO J. 6: 229-234), pMFa (Kutjan and Herskowitz, 1982. Cell 30: 933-943), pJRY88 (Schultz et al., 1987. Gene 54: 113-123), pYES2 (Invitrogen Corporation, San Diego, Calif), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the cc-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that. remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences, i.e., any one of SEQ ID NOS:2n−1, wherein n is an integer between 1 and 141, can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of a NOVX gene into which a deletion, addition or substitution has been introduced to thereby alter, e.g., functionally disrupt, the NOVX gene. The NOVX gene can be a human gene (e.g., the cDNA of any one of SEQ ID NOS:2n−1, wherein n is an integer between 1 and 141), but more preferably, is a non-human homologue of a human NOVX gene. For example, a mouse homologue of human NOVX gene of SEQ ID NOS:2n−1, wherein n is an integer between 1 and 141, can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those-enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in a NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease(possesses anti-microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds which bind to or modulate the activity of the membrane-bound form of a NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to a NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with a NOVX protein, wherein determining the ability of the test compound to interact with a NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with a NOVX target molecule. As used herein, a “target molecule” is a molecule with which a NOVX protein binds or interacts in nature, for example, a molecule on the surface of a cell which expresses a NOVX interacting protein, a molecule on the surface of a second cell, a molecule in the extracellular milieu, a molecule associated with the internal surface of a cell membrane or a cytoplasmic molecule. a NOVX target molecule can be a non-NOVX molecule or a NOVX protein or polypeptide of the invention. In one embodiment, a NOVX target molecule is a component of a signal transduction pathway that facilitates transduction of an extracellular signal (e.g. a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with a NOVX target molecule can be accomplished by one of the methods described above for determining direct binding. In one embodiment, determining the ability of the NOVX protein to bind to or interact with a NOVX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising a NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting a NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with a NOVX protein, wherein determining the ability of the test compound to interact with a NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof. Determining the ability of the test compound to modulate the activity of NOVX can be accomplished, for example, by determining the ability of the NOVX protein to bind to a NOVX target molecule by one of the methods described above for determining direct binding. In an alternative embodiment, determining the ability of the test compound to modulate the activity of NOVX protein can be accomplished by determining the ability of the NOVX protein further modulate a NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with a NOVX protein, wherein determining the ability of the test compound to interact with a NOVX protein comprises determining the ability of the NOVX protein to preferentially bind to or modulate the activity of a NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as -n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl--N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also likely to be involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of a NOVX sequence, i.e., of SEQ ID NOS:2n−1, wherein n is an integer between 1 and 141, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If coding sequences, such as those of SEQ ID NOS:2n−1, wherein n is an integer between 1 and 141, are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOVX protein, nucleic acid expression or activity. For example, mutations in a NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.) Yet another aspect of the invention pertains to monitoring the influence of agents (e.g., drugs, compounds) on the expression or activity of NOVX in clinical trials. These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NOS:2n−1, wherein n is an integer between 1 and 141, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in a NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding a NOVX-protein, or the misexpression of the NOVX gene. For example, such genetic lesions can be detected by ascertaining the existence of at least one of: (i) a deletion of one or more nucleotides from a NOVX gene; (ii) an addition of one or more nucleotides to a NOVX gene; (iii) a substitution of one or more nucleotides of a NOVX gene, (iv) a chromosomal rearrangement of a NOVX gene; (v) an alteration in the level of a messenger RNA transcript of a NOVX gene, (vi) aberrant modification of a NOVX gene, such as of the methylation pattern of the genomic DNA, (vii) the presence of a non-wild-type splicing pattern of a messenger RNA transcript of a NOVX gene, (viii) a non-wild-type level of a NOVX protein, (ix) allelic loss of a NOVX gene, and (x) inappropriate post-translational modification of a NOVX protein. As described herein, there are a large number of assay techniques known in the art which can be used for detecting lesions in a NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to a NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing sample and control nucleic acids, e.g., DNA or RNA to high-density arrays containing hundreds or thousands of oligonucleotide probes. See, e.g., Cronin, et al., 1996. Human Mutation 7: 244-255; Kozal, et al., 1996. Nat. Med. 2: 753-759. For example, genetic mutations in NOVX can be identified in two-dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on a NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders (The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers.) In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g. N-acetyltransferase 2 (NAT 2) and cytochrome Pregnancy Zone Protein Precursor enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C 19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of a NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostocodystrophy, and other diseases, disorders and conditions of the like.

These methods of treatment will be discussed more fully, below.

Diseases and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof, (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, a NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of a NOVX protein, a peptide, a NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of a NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering a NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The NOVX nucleic acids and proteins of the invention are useful in potential prophylactic and therapeutic applications implicated in a variety of disorders including, but not limited to: metabolic disorders, diabetes, obesity, infectious disease, anorexia, cancer-associated cancer, neurodegenerative disorders, Alzheimer's Disease, Parkinson's Disorder, immune disorders, hematopoietic disorders, and the various dyslipidemias, metabolic disturbances associated with obesity, the metabolic syndrome X and wasting disorders associated with chronic diseases and various cancers.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from: metabolic disorders, diabetes, obesity, infectious disease, anorexia, cancer-associated cachexia, cancer, neurodegenerative disorders, Alzheimer's Disease, Parkinson's Disorder, immune disorders, hematopoietic disorders, and the various dyslipidemias.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

The invention will be further described in the following examples, which do not limit the scope of the invention described in the claims.

### EXAMPLES

### Example A

### Polynucleotide And Polypeptide Sequences, And Homology Data

### Example 1

The NOV1 clone was analyzed, and the nucleotide and encoded polypeptide sequences are shown in Table 1A.

TABLE 1A
NOV1 Sequence Analysis

| | | |
|---|---|---|
| NOV1a, CG103945-02 | SEQ ID NO: 1 | 2414 bp |
| DNA Sequence | ORF Start: ATG at 1 | ORF Stop: TAG at 2401 |

ATGGCCCCCTCGGCCTGGGCCATTTGCTGGCTGCTAGGGGGCCTCCTGCTCCATGGGGGTAGCTCTGGCCCCA
GCCCCGGCCCCAGTGTGCCCCGCCTGCGGCTCTCCTACCGAGGAGCCGTGGTCCGAAAGCCTTCCAGCACCAT
GTGGATGGAAACATTTTCCAGATACCTCCTGTCTGCCAACCGCTCTGCCATCTTTCTGGGCCCCCAGGGCTCC
CTGAACCTCCAGGCCATGTACCTAGATGAGTACCGAGACCGCCTCTTTCTGGGTGGCCTGGACGCCCTCTACT
CTCTGCGGCTGGACCAGGCATGGCCAGATCCCCGGGAGGTCCTGTGGCCACCGCAGCCAGGACAGAGGGAGGA
GTGTGTTCGAAAGGGAAGAGATCCTTTGACAGAGTGCGCCAACTTCGTGCGGGTGCTACAGCCTCACAACCGG
ACCCACCTGCTAGCCTGTGGCACTGGGGCCTTCCAGCCCACCTGTGCCCTCATCACAGTTGGCCACCGTGGGG
AGCATGTGCTCCACCTGGAGCCTGGCAGTGTGGAAAGTGGCCGGGGGCGGTGCCCTCACGAGCCCAGCCGTCC
CTTTGCCAGCACCTTCATAGACGGGGAGCTGTACACGGGTCTCACTGCTGACTTCCTGGGGCGAGAGGCCATG
ATCTTCCGAAGTGGAGGTCCTCGGCCAGCTCTGCGTTCCGACTCTGACCAGAGTCTCTTGCACGACCCCCGGT
TTGTGATGGCCGCCCGGATCCCTGAGAACTCTGACCAGGACAATGACAAGGTGTACTTCTTCTTCTCGGAGAC
GGTCCCCTCGCCCGATGGTGGCTCGAACCATGTCACTGTCAGCCGCGTGGGCCGCGTCTGCGTGAATGATGCT
GGGGGCCAGCGGGTGCTGGTGAACAAATGGAGCACTTTCCTCAAGGCCAGGCTGGTCTGCTCGGTGCCCGGCC
CTGGTGGTGCCGAGACCCACTTTGACCAGCTAGAGGATGTGTTCCTGCTGTGGCCCAAGGCCGGGAAGAGCCT
CGAGGTGTACGCGCTGTTCAGCACCGTCAGTGCCGTGTTCCAGGGCTTCGCCGTCTGTGTGTACCACATGGCA
GACATCTGGGAGGTTTTCAACGGGCCCTTTGCCCACCGAGATGGGCCTCAGCACCAGTGGGGGCCCTATGGGG
GCAAGGTGCCCTTCCCTCGCCCTGGCGTGTGCCCCAGCAAGATGACCGCACAGCCAGGACGGCCTTTTGGCAG
CACCAAGGACTACCCAGATGAGGTGCTGCAGTTTGCCCGAGCCCACCCCCTCATGTTCTGGCCTGTGCGGCCT
CGACATGGCCGCCCTGTCCTTGTCAAGACCCACCTGGCCCAGCAGCTACACCAGATCGTGGTGGACCGCGTGG
AGGCAGAGGATGGGACCTACGATGTCATTTTCCTGGGGACTGACTCAGGGTCTGTGCTCAAAGTCATCGCTCT
CCAGGCAGGGGGCTCAGCTGAACCTGAGGAAGTGGTTCTGGAGGAGCTCCAGGTGTTTAAGGTGCCAACACCT
ATCACCGAAATGGAGATCTCTGTCAAAAGGCAAATGCTATACGTGGGCTCTCGGCTGGGTGTGGCCCAGCTGC
GGCTGCACCAATGTGAGACTTACGGCACTGCCTGTGCAGAGTGCTGCCTGGCCCGGGACCCATACTGTGCCTG
GGATGGTGCCTCCTGTACCCACTACCGCCCCAGCCTTGGCAACCGCCGGTTCCGCCGGCAGGACATCCGGCAC
GGCAACCCTGCCCTGCAGTGCCTGGGCCAGAGCCAGGAAGAAGAGGCAGTGGGACTTGTGGCAGCCACCATGG
TCTACGGCACGGAGCACAATAGCACCTTCCTGGAGTGCCTGCCCAAGTCTCCCCAGGCTGCTGTGCGCTGGCT
CTTGCAGAGGCCAGGGGATGAGGGGCCTGACCAGGTGAAGACGGACGAGCGAGTCTTGCACACGGAGCGGGGG
CTGCTGTTCCGCAGGCTTAGCCGTTTCGATGCGGGCACCTACACCTGCACCACTCTGGAGCATGGCTTCTCCC
AGACTGTGGTCCGCCTGGCTCTGGTGGTGATTGTGGCCTCACAGCTGGACAACCTGTTCCCTCCGGAGCCAAA
GCCAGAGGAGCCCCCAGCCCGGGGAGGCCTGGCTTCCACCCCACCCAAGGCCTGGTACAAGGACATCCTGCAG
CTCATTGGCTTCGCCAACCTGCCCCGGGTGGATGAGTACTGTGAGCGCGTGTGGTGCAGGGGCACCACGGAAT
GCTCAGGCTGCTTCCGGAGCCGGAGCCGGGGCAAGCAGGCCAGGGGCAAGAGCTGGGCAGGGCTGGAGCTAGG
CAAGAAGATGAAGAGCCGGGTGCATGCCGAGCACAATCGGACGCCCCGGGAGGTGGAGGCCACGTAGAAGGGG
GCAGA

| | | | |
|---|---|---|---|
| NOV1a, CG103945-02 | | | |
| Protein Sequence | SEQ ID NO: 2 | 800 aa | MW at 88800.3kD |

MAPSAWAICWLLGGLLLHGGSSGPSPGPSVPRLRLSYRGAVVRKPSSTMWMETFSRYLLSANRSAIFLGPQGS
LNLQAMYLDEYRDRLFLGGLDALYSLRLDQAWPDPREVLWPPQPGQREECVRKGRDPLTECANFVRVLQPHNR
THLLACGTGAFQPTCALITVGHRGEHVLHLEPGSVESGRGRCPHEPSRPFASTFIDGELYTGLTADFLGREAM
IFRSGGPRPALRSDSDQSLLHDPRFVMAARIPENSDQDNDKVYFFFSETVPSPDGGSNHVTVSRVGRVCVNDA
GGQRVLVNKWSTFLKARLVCSVPGPGGAETHFDQLEDVFLLWPKAGKSLEVYALFSTVSAVFQGFAVCVYHMA
DIWEVFNGPFAHRDGPQHQWGPYGGKVPFPRPGVCPSKMTAQPGRPFGSTKDYPDEVLQFARAHPLMFWPVRP
RHGRPVLVKTHLAQQLHQIVVDRVEAEDGTYDVIFLGTDSGSVLKVIALQAGGSAEPEEVVLEELQVFKVPTP
ITEMEISVKRQMLYVGSRLGVAQLRLHQCETYGTACAECCLARDPYCAWDGASCTHYRPSLGKRRFRRQDIRH
GNPALQCLGQSQEEEAVGLVAATMVYGTEHNSTFLECLPKSPQAAVRWLLQRPGDEGPDQVKTDERVLHTERG
LLFRRLSRFDAGTYTCTTLEHGFSQTVVRLALVVIVASQLDNLFPPEPKPEEPPARGGLASTPPKAWYKDILQ
LIGFANLPRVDEYCERVWCRGTTECSGCFRSRSRGKQARGKSWAGLELGKKMKSRVHAEHNRTPREVEAT

| | | |
|---|---|---|
| NOV1b, CG103945-01 | SEQ ID NO: 3 | 4700 bp |
| DNA Sequence | ORF Start: ATG at 1 | ORF Stop: TAG at 2347 |

ATGGCCCCCTCGGCCTGGGCCATTTGCTGGCTGCTAGGGGGCCTCCTGCTCCATGGGGGTAGCTCTGGCCCCA
GCCCCGGCCCCAGTGTGCCCCGCCTGCGGCTCTCCTACCGAGACCTCCTGTCTGCCAACCGCTCTGCCATCTT
TCTGGGCCCCCAGGGCTCCCTGAACCTCCAGGCCATGTACCTAGATGAGTACCGAGACCGCCTCTTTCTGGGT
GGCCTGGACGCCCTCTACTCTCTGCGGCTGGACCAGGCATGGCCAGATCCCCGGGAGGTCCTGTGGCCACCGC
AGCCAGGACAGAGGGAGGAGTGTGTTCGAAAGGGAAGAGATCCTTTGACAGAGTGCGCCAACTTCGTGCGGGT
GCTACAGCCTCACAACCGGACCCACCTGCTAGCCTGTGGCACTGGGGCCTTCCAGCCCACCTGTGCCCTCATC
ACAGTTGGCCACCGTGGGGAGCATGTGCTCCACCTGGAGCCTGGCAGTGTGGAAAGTGGCCGGGGGCGGTGCC
CTCACGAGCCCAGCCGTCCCTTTGCCAGCACCTTCATAGACGGGGAGCTGTACACGGGTCTCACTGCTGACTT
CCTGGGGCGAGAGGCCATGATCTTCCGAAGTGGAGGTCCTCGGCCAGCTCTGCGTTCCGACTCTGACCAGAGT
CTCTTGCACGACCCCCGGTTTGTGATGGCCGCCCGGATCCCTGAGAACTCTGACCAGGACAATGACAAGGTGT
ACTTCTTCTTCTCGGAGACGGTCCCCTCGCCCGATGGTGGCTCGAACCATGTCACTGTCAGCCGCGTGGGCCG
CGTCTGCGTGAATGATGCTGGGGGCCAGCGGGTGCTGGTGAACAAATGGAGCACTTTCCTCAAGGCCAGGCTG
GTCTGCTCGGTGCCCGGCCCTGGTGGTGCCGAGACCCACTTTGACCAGCTAGAGGATGTGTTCCTGCTGTGGC
CCAAGGCCGGGAAGAGCCTCGAGGTGTACGCGCTGTTCAGCACCGTCAGTGCCGTGTTCCAGGGCTTCGCCGT
CTGTGTGTACCACATGGCAGACATCTGGGAGGTTTTCAACGGGCCCTTTGCCCACCGAGATGGGCCTCAGCAC
CAGTGGGGGCCCTATGGGGGCAAGGTGCCCTTCCCTCGCCCTGGCGTGTGCCCCAGCAAGATGACCGCACAGC
CAGGACGGCCTTTTGGCAGCACCAAGGACTACCCAGATGAGGTGCTGCAGTTTGCCCGAGCCCACCCCCTCAT
GTTCTGGCCTGTGCGGCCTCGACATGGCCGCCCTGTCCTTGTCAAGACCCACCTGGCCCAGCAGCTACACCAG
ATCGTGGTGGACCGCGTGGAGGCAGAGGATGGGACCTACGATGTCATTTTCCTGGGGACTGACTCAGGGTCTG
TGCTCAAAGTCATCGCTCTCCAGGCAGGGGGCTCAGCTGAACCTGAGGAAGTGGTTCTGGAGGAGCTCCAGGT
GTTTAAGGTGCCAACACCTATCACCGAAATGGAGATCTCTGTCAAAAGGCAAATGCTATACGTGGGCTCTCGG
CTGGGTGTGGCCCAGCTGCGGCTGCACCAATGTGAGACTTACGGCACTGCCTGTGCAGAGTGCTGCCTGGCCC
GGGACCCATACTGTGCCTGGGATGGTGCCTCCTGTACCCACTACCGCCCCAGCCTTGGCAAGCGCCGGTTCCG
CCGGCAGGACATCCGGCACGGCAACCCTGCCCTGCAGTGCCTGGGCCAGAGCCAGGAAGAAGAGGCAGTGGGA
CTTGTGGCAGCCACCATGGTCTACGGCACGGAGCACAATAGCACCTTCCTGGAGTGCCTGCCCAAGTCTCCCC
AGGCTGCTGTGCGCTGGCTCTTGCAGAGGCCAGGGGATGAGGGGCCTGACCAGGTGAAGACGGACGAGCGAGT
CTTGCACACGGAGCGGGGGCTGCTGTTCCGCAGGCTTAGCCGTTTCGATGCGGGCACCTACACCTGCACCACT
CTGGAGCATGGCTTCTCCCAGACTGTGGTCCGCCTGGCTCTGGTGGTGATTGTGGCCTCACAGCTGGACAACC
TGTTCCCTCCGGAGCCAAAGCCAGAGGAGCCCCCAGCCCGGGGAGGCCTGGCTTCCACCCCACCCAAGGCCTG
GTACAAGGACATCCTGCAGCTCATTGGCTTCGCCAACCTGCCCCGGGTGGATGAGTACTGTGAGCGCGTGTGG
TGCAGGGGCACCACGGAATGCTCAGGCTGCTTCCGGAGCCGGAGCCGGGGCAAGCAGGCCAGGGGCAAGAGCT
GGGCAGGGCTGGAGCTAGGCAAGAAGATGAAGAGCCGGGTGCATGCCGAGCACAATCGGACGCCCCGGGAGGT
GGAGGCCACGTAGAAGGGGGCAGAGGAGGGGTGGTCAGGATGGGCTGGGGGGCCCACTAGCAGCCCCCAGCAT
CTCCCACCCACCCAGCTAGGGCAGAGGGGTCAGCATGTCTGTTTGCCTCTTAGAGACAGGTGTCTCTGCCCCC
ACACCGCTACTGGGGTCTAATGGAGGGGCTGGGTTCTTGAAGCCTGTTCCCTGCCCTTCTCTGTGCTCTTAGA
CCCAGCTGGAGCCAGCACCCTCTGGCTGCTGGCAGCCCCAAGGGATCTGCCATTTGTTCTCAGAGATGGCCTG
GCTTCCGCAACACATTTCCGGGTGTGCCCAGAGGCAAGAGGGTTGGGTGGTTCTTTCCCAGCCTACAGAACAA
TGGCCATTCTGAGTGACCCTCAGAGTGGGTGTGTGGGTGCGTCTAGGGGGTATCCCGGTAGGGGGCCTGCAGG
GAGCCAGAGGGTGGAAATGGCCTCTAAGCTAGCACCCCGTAAGAAGAGCCTACCTGACCGACTTGGGGAGGGA
ACACAGAGGTGTTGGGAAGGTGGAGCAACAATGCACCTCCCCTCCTGTCGCGCCGTGATATCTTGGTGGCTCC
CTGCCACTGCCCACCGCCTCTTCTCCATCTGAGAATCACGGAGAGCTGTAGATAATCTAGAGGCATAGACTGC
TAGAGCCCCCAGGATCTGGGGTGGTCAGGGCTCAGGCTTCACTTTGTAAACCAGGTGGGGGCATCTCACAGC
CTGACTTCCCTTCCCCAGGCCAGGGTTGCTGGGATGCCTGCCCCTCCTGAGAGGACCCCCTCCCCATTGTCAG
GCTCTCCATGTCCACGAGCGGGGAGGGGTGGGTTCTGGGGCATTGTTGTCCCTTGTGTCTGTGGACTAGAGAT
AGGGTGGGGGAGCTGGGGAAGGGTGCAGGCGGGAAGAGTGGGCTGTCTTTCCCAGGGTGATGCAAGCATGCCG
CAGCCCTGGAGGCTGGGAATGTGGAGGCTCTGTGAGCCCTGCAGCCC
TCAGAATCAGGGCCAGGGATGCAGAAGATTGAGAGGATATGGAGATGGATAGAGGGCAGGAGACCCTTAGGAT
AGATTGTGGGACCCAGGCAGGAACAGGTGTCCACAAGAACTCAGGATGGCATCAGTTAGCTCAGAAGCCACCT
GGAAGACCCAGTGTTTCCATCTCTGGAATCTCTGTTTTATGCTAAATGGATTTAGGAAGACTGTTTTTCTTTT
AAGGGGGAAACAAGGTAGAGAAAAGGACGAAGAAGTGTAAGTCCCGCTGATTCTCGGGGGTAAGGCTCGGATG
GCAAGGACGCGTTCTGCCTGGGCATGTAGGGGAGGTGTTTTTGCCATCACCAGTTTCTCAGGCTGGGGAGCAC
AGAGGGGAGGAGGAGGACTAAATGAAAAGTTGTTCCCAGCCTGCACATGAACACATTCATGACACACAAAACT
GGCTGGAAGGAGATAAGAGCACTGGGTTTGAGATTCCCTCCATTAAAACAACCAAGACAAAGAAAGGAGGGGA
AAAAAAGATAAAAAGCAAGCCAGGGTTCCCTGCCCTATTGAAACTCAAACCCAGACTGCCTTGGGTTTTATCT
TTCCCTTACCCCTGGCACCTCCAGAGAACTGGGACCTGAAATAGTCCCTCCGTTCTCCCCTTTGACCATGTAA
TAAATGAACCAGAAGCACTGAGATTAACCTATCAACGCCCTGAGAAGCCTTCCAGCCTGCGGTGCTGTCTGCT
GGGAGGTCAGCTGGTCAAGGCAGAGGAGGAGAGGAGGAAAGGATGGGGGCTGAAGAGCAGAAGGGAGGGGAGA
CAGAGGGGATTAAAGAGGGGAGGAGAGAGTGCAGAGCTCCAGGAAAGGGTATCAGAGCTGCAGCCAGCTCTGC
CCTCTACCCTAGGGAGGCCAGAAAGACACAAACAGCCCTCCGGGCCTTTACGCTGGACTCTGGCTTGGCAGGC
TCCAGGCAGGGTCCTCTGGGAAGTTACTCTAGAAAACGAAGGGAGGAGGAGCACAAGATCCTCAGCAACGAAC
ACCTGCACTTAGAAAAAGTGGACAGCTTCTGCCAACCACACCCTACCCATGGTACTGTATGCTATTAACTCCT
GGAAACGCCCCGTAAATGCGAGTTGTTTTTGTATTTGTGTGTTGAGATGGGCCTTGTGGTTTCTCTGTACTCA
GAGCACATTTCTTGTAATTACTATTGTTATTTTTATTGTCATGACTGCCCCTGAGCTCTGGTGAGAAAAGCTG
AATTTACAAGGAAAGGGATGAAGTTAATATTTGCATCACATAATTATATCATTACTGTGTATCTGTGTATTGT
ACTAAATGGACTGATGCTGCGCACATGAGCTGAAAATGAAGAGCCCTCCCATCC

| | | | |
|---|---|---|---|
| NOV1b, CG103945-01 | | | |
| Protein Sequence | SEQ ID NO: 4 | 782 aa | MW at 86699.9kD |

MAPSAWAICWLLGGLLLHGGSSGPSPGPSVPRLRLSYRDLLSANRSAIFLGPQGSLNLQAMYLDEYRDRLFLG
GLDALYSLRLDQAWPDPREVLWPPQPGQREECVRKGRDPLTECANFVRVLQPHNRTHLLACGTGAFQPTCALI
TVGHRGEHVLHLEPGSVESGRGRCPHEPSRPFASTFIDGELYTGLTADFLGREAMIFRSGGPRPALRSDSDQS
LLHDPRFVMAARIPENSDQDNDKVYFFFSETVPSPDGGSNHVTVSRVGRVCVNDAGGQRVLVNKWSTFLKARL
VCSVPGPGGAETHFDQLEDVFLLWPKAGKSLEVYALFSTVSAVFQGFAVCVYHMADIWEVFNGPFAHRDGPQH
QWGPYGGKVPFPRPGVCPSKMTAQPGRPFGSTKDYPDEVLQFARAHPLMFWPVRPRHGRPVLVKTHLAQQLHQ
IVVDRVEAEDGTYDVIFLGTDSGSVLKVIALQAGGSAEPEEVVLEELQVFKVPTPITEMEISVKRQMLYVGSR
LGVAQLRLHQCETYGTACAECCLARDPYCAWDGASCTHYRPSLGKRRFRRQDIRHGNPALQCLGQSQEEEAVG
LVAATMVYGTEHNSTFLECLPKSPQAAVRWLLQRPGDEGPDQVKTDERVLHTERGLLFRRLSRFDAGTYTCTT
LEHGFSQTVVRLALVVIVASQLDNLFPPEPKPEEPPARGGLASTPPKAWYKDILQLIGFANLPRVDEYCERVW
CRGTTECSGCFRSRSRGKQARGKSWAGLELGKKMKSRVHAEHNRTPREVEAT

A ClustalW comparison of the above protein sequences yields the following sequence alignment shown in Table 1B.

TABLE 1B
Comparison of the NOV1 protein sequences.

| | |
|---|---|
| NOV1a | MAPSAWAICWLLGGLLLHGGSSGPSPGPSVPRLRLSYRGAVVRKPSSTMWMETFSRYLLS |
| NOV1b | MAPSAWAICWLLGGLLLHGGSSGPSPGPSVPRLRLSYR-------------D-----LLS |
| NOV1a | ANRSAIFLGPQGSLNLQAMYLDEYRDRLFLGGLDALYSLRLDQAWPDPREVLWPPQPGQR |
| NOV1b | ANRSAIFLGPQGSLNLQAMYLDEYRDRLFLGGLDALYSLRLDQAWPDPREVLWPPQPGQR |
| NOV1a | EECVRKGRDPLTECANFVRVLQPHNRTHLLACGTGAFQPTCALITVGHRGEHVLHLEPGS |
| NOV1b | EECVRKGRDPLTECANFVRVLQPHNRTHLLACGTGAFQPTCALITVGHRGEHVLHLEPGS |
| NOV1a | VESGRGRCPHEPSRPFASTFIDGELYTGLTADFLGREAMIFRSGGPRPALRSDSDQSLLH |
| NOV1b | VESGRGRCPHEPSRPFASTFIDGELYTGLTADFLGREAMIFRSGGPRPALRSDSDQSLLH |
| NOV1a | DPRFVMAARIPENSDQDNDKVYFFFSETVPSPDGGSNHVTVSRVGRVCVNDAGGQRVLVN |
| NOV1b | DPRFVMAARIPENSDQDNDKVYFFFSETVPSPDGGSNHVTVSRVGRVCVNDAGGQRVLVN |
| NOV1a | KWSTFLKARLVCSVPGPGGAETHFDQLEDVFLLWPKAGKSLEVYALFSTVSAVFQGFAVC |
| NOV1b | KWSTFLKARLVCSVPGPGGAETHFDQLEDVFLLWPKAGKSLEVYALFSTVSAVFQGFAVC |
| NOV1a | VYHMADIWEVFNGPFAHRDGPQHQWGPYGGKVPFPRPGVCPSKMTAQPGRPFGSTKDYPD |
| NOV1b | VYHMADIWEVFNGPFAHRDGPQHQWGPYGGKVPFPRPGVCPSKMTAQPGRPFGSTKDYPD |
| NOV1a | EVLQFARAHPLMFWPVRPRHGRPVLVKTHLAQQLHQIVVDRVEAEDGTYDVIFLGTDSGS |
| NOV1b | EVLQFARAHPLMFWPVRPRHGRPVLVKTHLAQQLHQIVVDRVEAEDGTYDVIFLGTDSGS |
| NOV1a | VLKVIALQAGGSAEPEEVVLEELQVFKVPTPITEMEISVKRQMLYVGSRLGVAQLRLHQC |
| NOV1b | VLKVIALQAGGSAEPEEVVLEELQVFKVPTPITEMEISVKRQMLYVGSRLGVAQLRLHQC |
| NOV1a | ETYGTACAECCLARDPYCAWDGASCTHYRPSLGKRRFRRQDIRHGNPALQCLGQSQEEEA |
| NOV1b | ETYGTACAECCLARDPYCAWDGASCTHYRPSLGKRRFRRQDIRHGNPALQCLGQSQEEEA |
| NOV1a | VGLVAATMVYGTEHNSTFLECLPKSPQAAVRWLLQRPGDEGPDQVKTDERVLHTERGLLF |
| NOV1b | VGLVAATMVYGTEHNSTFLECLPKSPQAAVRWLLQRPGDEGPDQVKTDERVLHTERGLLF |
| NOV1a | RRLSRFDAGTYTCTTLEHGFSQTVVRLALVVIVASQLDNLFPPEPKPEEPPARGGLASTP |
| NOV1b | RRLSRFDAGTYTCTTLEHGFSQTVVRLALVVIVASQLDNLFPPEPKPEEPPARGGLASTP |
| NOV1a | PKAWYKDILQLIGFANLPRVDEYCERVWCRGTTECSGCFRSRSRGKQARGKSWAGLELGK |
| NOV1b | PKAWYKDILQLIGFANLPRVDEYCERVWCRGTTECSGCFRSRSRGKQARGKSWAGLELGK |
| NOV1a | KMKSRVHAEHNRTPREVEAT |
| NOV1b | KMKSRVHAEHNRTPREVEAT |
| NOV1a | (SEQ ID NO: 2) |
| NOV1b | (SEQ ID NO: 4) |

Further analysis of the NOV1a protein yielded the following properties shown in Table 1C.

TABLE 1C
Protein Sequence Properties NOV1a

| SignalP | Cleavage site between residues 23 and 24 | | | |
|---|---|---|---|---|
| analysis: | | | | |
| PSORT II | PSG: a new signal peptide prediction method | | | |
| analysis: | N-region: length 0; pos. chg 0; neg. chg 0 | | | |
| | H-region: length 31; peak value 9.35 | | | |
| | PSG score: 4.95 | | | |
| | GvH: von Heijne's method for signal seq. recognition | | | |
| | GvH score (threshold: −2.1): 1.50 | | | |
| | possible cleavage site: between 22 and 23 | | | |
| | >>> Seems to have a cleavable signal peptide (1 to 22) | | | |
| | ALOM: Klein et al's method for TM region allocation | | | |
| | Init position for calculation: 23 | | | |
| | Tentative number of TMS(s) for the threshold 0.5: 3 | | | |
| | Number of TMS(s) for threshold 0.5: 1 | | | |
| | INTEGRAL | Likelihood = | −2.02 | Transmembrane 345-361 |
| | PERIPHERAL | Likelihood = | 2.86 (at 150) | |
| | ALOM score: −2.02 (number of TMSs: 1) | | | |
| | MTOP: Prediction of membrane topology (Hartmann et al.) | | | |
| | Center position for calculation: 11 | | | |
| | Charge difference: 0.5 C(1.5)-N(1.0) | | | |
| | C > N: C-terminal side will be inside | | | |
| | >>>Caution: Inconsistent mtop result with signal peptide | | | |
| | >>> membrane topology: type 1a (cytoplasmic tail 362 to 800) | | | |
| | MITDISC: discrimination of mitochondrial targeting seq | | | |
| | R content: | 4 | Hyd Moment(75): | 2.13 |
| | Hyd Moment(95): | 2.46 | G content: | 7 |
| | D/E content: | 1 | S/T content: | 9 |

Score: −2.94
Gavel: prediction of cleavage sites for mitochondrial preseq
R-2 motif at 73 NRS|AI
NUCDISC: discrimination of nuclear localization signals
pat4: none
pat7: PSLGKRR (3) at 570
bipartite: none
content of basic residues: 11.4%
NLS Score: −0.22
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: too long tail
Dileucine motif in the tail: found
LL at 633
LL at 658
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 89
COIL: Lupas's algorithm to detect coiled-coil regions
total: 0 residues
Final Results (k = 9/23):
44.4%: endoplasmic reticulum
22.2%: Golgi
22.2%: extracellular, including cell wall
11.1%: plasma membrane
>> prediction for CG103945-02 is end (k = 9)

A search of the NOV1a protein against the Geneseq database, a proprietary database that contains sequences published in patents and patent publication, yielded several homologous proteins shown in Table 1D.

TABLE 1D
Geneseq Results for NOV1a

| | | NOV1a | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| AAG65620 | Novel human protein (NHP) sequence - | 1 . . . 800 | 781/800 (97%) | 0.0 |
| | Homo sapiens, 782 aa. | 1 . . . 782 | 781/800 (97%) | |
| | [WO200170806-A2, 27 SEP. 2001] | | | |
| AAG65619 | Novel human protein (NHP) sequence - | 1 . . . 800 | 781/800 (97%) | 0.0 |
| | Homo sapiens, 875 aa. | 94 . . . 875 | 781/800 (97%) | |
| | [WO200170806-A2, 27 SEP. 2001] | | | |
| AAB23609 | Human secreted protein SEQ ID NO: | 1 . . . 800 | 781/800 (97%) | 0.0 |
| | 18 - Homo sapiens, 782 aa. | 1 . . . 782 | 781/800 (97%) | |
| | [WO200049134-A1, 24 AUG. 2000] | | | |
| AAB23636 | Human secreted protein SEQ ID NO: | 1 . . . 800 | 781/803 (97%) | 0.0 |
| | 92 - Homo sapiens, 785 aa. | 1 . . . 785 | 781/803 (97%) | |
| | [WO200049134-A1, 24 AUG. 2000] | | | |
| AAG78481 | Human ZSMF-16 - Homo sapiens, | 1 . . . 800 | 778/800 (97%) | 0.0 |
| | 779 aa. [US2001049432-A1, | 1 . . . 779 | 779/800 (97%) | |
| | 06 DEC. 2001] | | | |

In a BLAST search of public sequence databases, the NOV1a protein was found to have homology to the proteins shown in the BLASTP data in Table 1E.

TABLE 1E
Public BLASTP Results for NOV1a

| | | NOV1a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9NS98 | Semaphorin sem2 (FLJ00014 | 1 . . . 800 | 781/800 (97%) | 0.0 |
| | protein) - Homo sapiens (Human), | 1 . . . 782 | 781/800 (97%) | |
| | 782 aa. | | | |
| CAC42673 | Sequence 1 from Patent WO0140278 - | 1 . . . 800 | 778/800 (97%) | 0.0 |
| | Homo sapiens (Human), 779 aa. | 1 . . . 779 | 779/800 (97%) | |
| Q9QX23 | Semaphorin M-SemaK - Mus | 1 . . . 795 | 399/805 (49%) | 0.0 |
| | musculus (Mouse), 775 aa. | 1 . . . 770 | 525/805 (64%) | |
| P70275 | Semaphorin 3E precursor | 1 . . . 795 | 398/805 (49%) | 0.0 |
| | (Semaphorin H) (Sema H) - Mus | 1 . . . 770 | 524/805 (64%) | |
| | musculus (Mouse), 775 aa. | | | |
| O42237 | Semaphorin 3E precursor | 1 . . . 797 | 398/806 (49%) | 0.0 |
| | (Collapsin-5) (COLL-5) - Gallus | 5 . . . 782 | 519/806 (64%) | |
| | gallus (Chicken), 785 aa. | | | |

PFam analysis predicts that the NOV1a protein contains the domains shown in the Table 1F.

TABLE 1F
Domain Analysis of NOV1a

| | | Identities/ | |
|---|---|---|---|
| Pfam | | Similarities | Expect |
| Domain | NOV1a Match Region | for the Matched Region | Value |
| Sema | 76 . . . 521 | 217/497 (44%) | 1.3e−176 |
| | | 360/497 (72%) | |
| PSI | 539 . . . 622 | 14/101 (14%) | 0.76 |
| | | 56/101 (55%) | |
| ig | 614 . . . 675 | 15/66 (23%) | 0.0061 |
| | | 45/66 (68%) | |

### Example 2

The NOV2 clone was analyzed, and the nucleotide and encoded polypeptide sequences are shown in Table 2A.

TABLE 2A
NOV2 Sequence Analysis

| | | |
|---|---|---|
| NOV2a, CG106951-01 | SEQ ID NO: 5 | 6408 bp |
| DNA Sequence | ORF Start: ATG at 1400 | ORF Stop: TGA at 5456 |

CCTGGGACTCTGGGAGAATGGTCCAGAGCTCATTGTCCTTGTTGATAAAATGATAGATTTGGACTCAATATCC
CATGCTGCCTCTTCCAACTTGATTTTTACCCCAGACTGGGCTACCAGACTGGTATGCCCACACATGCCCGTTT
CCTTTCTTTTCTTCTCTGCATCTCTGCCTTTGTGTCCAGAGCGTGTTTTCCCTTTGCAAGTTTCTCTCCATTC
TGCACATTATGAGTTTCAGCATTTCTGTTGCCCTAGAAAGTCTATCTTTGAGATCTTGCACTGTTTCTCTTTT
TACAGTGTCTCATAAACTCCCTTCTTGGATTCAGAACCACCCTTTCTTTCCCATTATCCTGTCAAACTGCTTC
TTGCCATGGTCCAGGGGTAGGAGGATGGCAGGCAGGAGGTGCTTCTCTGGGGCTCTTAGTGTCTCAATTCTTC
TGCTTTATCTGGGTTTTCCTTTACCCAGAATTTTATTATGTAAAATGCTTCACTCAGACTTTGTTCTAATTAT
CCAATTTTTGGCATACTCTAGAAAGTCTTTTGATATTTTCCTTCCTCCAACTTATCTATTTTTATTTCATAGT
TCTCTTTGGTTATCTCTTAGAATCACACTTTCCTGGTTTTAATTTTTCAAATCCTTTGTCTTTCTCACTCGTT
CTTAGGTCACCTTTTTTTACATTTTCAAATATATTTTTTGTTCAGCAGAGGGCTCCCTTCCCATCCCTCTTGC
AGCCCGGGCAGCTAGGATTTGAAGCTTGCCCCTTGAATCTTTCTCTCCCGCCTTCTAGCCATCAGAAACACTA
GATCACTTAAACTTGTAAACAATTCGGCCTCGCTCCTTGTGATTGCGCTAAACCTTCCGTCCTCAGCTGAGAA
CGCTCCACCACCTCCCCGGATCGCTCATCTCTTGGCTGCCCTCCCACTGTTCCTGATGTTATTTTACTCCCCG
TATCCCCTACTCGTTCTTCACAATTCTGTAGGGTGCGTATTACTAACCCCAGTTTACAGCTGAGGAAACTGAG
GCTTGGAGAGGTTCGCTCGGTATCGTACAGTTTGCAAGGTTAACCCTAATCCGGCCAGTTCTGGCTTTCCAGC
CCAGCCCAGCAGCCTAGCCTCCCTCTCTGCCGCTGCAGGTTATAACGGCTCTCCCCCGTTTTACACGAGGTCC
CTTCCCCTTCAAATCCACAGGCAGGAAGATCGTTCCGAACTGACGGGGCTGGGGAATGTGGGAGTCCGGAGTG
GGGTTTGGGGGAGCTTCCTCAGGCCCTGAGTGTTGGGGTGGGCAGGCCGCGCCGATGGCCCTCGGGGATGTCA
CATTCGAGATGGGGTGACCGAGAACGGCAAGGCGGGATGTGGCAAACGGCGGCAAGTGCTCGGAGTCCTAGGT
CTTGCCGCCGGAATGCCGGCCGGGGAAGGGGCTTCGGCCCACCGGGCTGGTCACCACACTCGGCAGGCCCGGG
GCGGGAGTCGGCCGAGCAGCCGCGGGATGCAGGGCGCCCCCTCGCGCTCCTCCGCGCGCCTCGAGGCTGGCGG
GTGCAGCGCCCGCCGCGGCAGGTCTGCTCCAGCCCCCTCCTCTTTTTCGCTCCCGCTCCCCTCCTTCTCTCCC
TTTGCTTGCAACTCCTCCCCCACCGCCCCCTCCCTCCTTCTGCTCCCGCGGTCTCCTCCTCCCTGCTCTCTCC
GAGCGCCGGGTCGGGAGCTAGTTGGAGCGCGGGGGTTGGTGCCAGAGCCCAGCTCCGCCGAGCCGGGCGGGTC
GGCAGCGCATCCAGCGGCTGCTGGGAGCCCGAGCGCAGCGGGCGCGGGCCCGGGTGGGGACTGCACCGGAGCG
CTGAGAGCTGGAGGCCGTTCCTGCGCGGCCGCCCCATTCCCAGACCGGCCGCCAGCCCATCTGGTTAGCTCCC
GCCGCTCCGCGCCGCCCGGGAGTCGGGAGCCGCGGGGAACCGGGCACCTGCACCCGCCTCTGGGAGTGAGTGG
TTCCAGCTGGTGCCTGGCCTGTGTCTCTTGGATGCCCTGTGGCTTCAGTCCGTCTCCTGTTGCCCACCACCTC
GTCCCTGGGCCGCCTGATACCCCAGCCCAACAGCTAAGGTGTGGATGGACAGTAGGGGGCTGGCTTCTCTCAC
TGGTCAGGGGTCTTCTCCCCTGTCTGCCTCCCGGAGCTAGGACTGCAGAGGGGCCTATCATGGTGCTTGCAGG
CCCCCTGGCTGTCTCGCTGTTGCTGCCCAGCCTCACACTGCTGGTGTCCCACCTCTCCAGCTCCCAGGATGTC
TCCAGTGAGCCCAGCAGTGAGCAGCAGCTGTGCGCCCTTAGCAAGCACCCCACCGTGGCCTTTGAAGACCTGC
AGCCGTGGGTCTCTAACTTCACCTACCCTGGAGCCCGGGATTTCTCCCAGCTGGCTTTGGACCCCTCCGGGAA
CCAGCTCATCGTGGGAGCCAGGAACTACCTCTTCAGACTCAGCCTTGCCAATGTCTCTCTTCTTCAGGCCACA
GAGTGGGCCTCCAGTGAGGACACGCGCCGCTCCTGCCAAAGCAAAGGGAAGACTGAGGAGGAGTGTCAGAACT
ACGTGCGAGTCCTGATCGTCGCCGGCCGGAAGGTGTTCATGTGTGGAACCAATGCCTTTTCCCCCATGTGCAC
CAGCAGACAGGTGGGGAACCTCAGCCGGACTACTGAGAAGATCAATGGTGTGGCCCGCTGCCCCTATGACCCA
CGCCACAACTCCACAGCTGTCATCTCCTCCCAGGGGGAGCTCTATGCAGCCACGGTCATCGACTTCTCAGGTC
GGGACCCTGCCATCTACCGCAGCCTGGGCAGTGGGCCACCGCTTCGCACTGCCCAATATAACTCCAAGTGGCT
TAATGAGCCAAACTTCGTGGCAGCCTATGATATTGGGCTGTTTGCATACTTCTTCCTGCGGGAGAACGCAGTG
GAGCACGACTGTGGACGCACCGTGTACTCTCGCGTGGCCCGCGTGTGCAAGAATGACGTGGGGGGCCGATTCC
TGCTGGAGGACACATGGACCACATTCATCAAGGCCCGGCTCAACTGCTCCCGCCCGGGCGAGGTCCCCTTCTA
CTATAACGAGCTGCAGAGTGCCTTCCACTTGCCAGAGCAGGACCTCATCTATGGAGTTTTCACAACCAACGTA
AACAGCATCGCGGCTTCTGCTGTCTGCGCCTTCAACCTCAGTGCTATCTCCCAGGCTTTCAATGGCCCATTTC
GCTACCAGGAGAACCCCAGGGCTGCCTGGCTCCCCATAGCCAACCCCATCCCCAATTTCCAGTGTGGCACCCT
GCCTGAGACCGGTCCCAACGAGAACCTGACGGAGCGCAGCCTGCAGGACGCGCAGCGCCTCTTCCTGATGAGC
GAGGCCGTGCAGCCGGTGACACCCGAGCCCTGTGTCACCCAGGACAGCGTGCGCTTCTCACACCTCGTGGTGG
ACCTGGTGCAGGCTAAAGACACGCTCTACCATGTACTCTACATTGGCACCGAGTCGGGCACCATCCTGAAGGC
GCTGTCCACGGCGAGCCGCAGCCTCCACGGCTGCTACCTGGAGGAGCTGCACGTGCTGCCCCCCGGGCGCCGC
GAGCCCCTGCGCAGCCTGCGCATCCTGCACAGCGCCCGCGCGCTCTTCGTGGGGCTGAGAGACGGCGTCCTGC
GGGTCCCACTGGAGAGGTGCGCCGCCTACCGCAGCCAGGGGGCATGCCTGGGGGCCCGGGACCCGTACTGTGG
CTGGGACGGGAAGCAGCAACGTTGCAGCACACTCGAGGACAGCTCCAACATGAGCCTCTGGACCCAGAACATC
ACCGCCTGTCCTGTGCGGAATGTGACACGGGATGGGGGCTTCGGCCCATGGTCACCATGGCAACCATGTGAGC
ACTTGGATGGGGACAACTCAGGCTCTTGCCTGTGTCGAGCTCGATCCTGTGATTCCCCTCGACCCCGCTGTGG
GGGCCTTGACTGCCTGGGGCCAGCCATCCACATCGCCAACTGCTCCAGGAATGGGGCGTGGACCCCGTGGTCA
TCGTGGGCGCTGTGCAGCACGTCCTGTGGCATCGGCTTCCAGGTCCGCCAGCGAAGTTGCAGCAACCCTGCTC
CCCGCCACGGGGGCCGCATCTGCGTGGGCAAGAGCCGGGAGGAACGGTTCTGTAATGAGAACACGCCTTGCCC
GGTGCCCATCTTCTGGGCTTCCTGGGGCTCCTGGAGCAAGTGCAGCAGCAACTGTGGAGGGGGCATGCAGTCG
CGGCGTCGGGCCTGCGAGAACGGCAACTCCTGCCTGGGCTGCGGCGTGGAGTTCAAGACGTGCAACCCCGAGG
GCTGCCCCGAAGTGCGGCGCAACACCCCCTGGACGCCGTGGCTGCCCGTGAACGTGACGCAGGGCGGGGCACG
GCAGGAGCAGCGGTTCCGCTTCACCTGCCGCGCGCCCCTTGCAGACCCGCACGGCCTGCAGTTCGGCAGGAGA
AGGACCGAGACGAGGACCTGTCCCGCGGACGGCTCCGGCTCCTGCGACACCGACGCCCTGGTGGAGGACCTCC
TGCGCAGCGGGAGCACCTCCCCGCACACGGTGAGCGGGGGCTGGGCCGCCTGGGGCCCGTGGTCGTCCTGCTC
CCGGGACTGCGAGCTGGGCTTCCGCGTCCGCAAGAGAACGTGCACTAACCCGGAGCCCCGCAACGGGGGCCTG
CCCTGCGTCGGCGATGCTGCCGAGTACCAGGACTGCAACCCCCAGGCTTGCCCAGTTCGGGGTGCTTGGTCCT
GCTGGACCTCATGGTCTCCATGCTCAGCTTCCTGTGGTGGGGGTCACTATCAACGCACCCGTTCCTGCACCAG
CCCCGCACCCTCCCCAGGTGAGGACATCTGTCTCGGGCTGCACACGGAGGAGGCACTATGTGCCACACAGGCC
TGCCCAGAAGGCTGGTCGCCCTGGTCTGAGTGGAGTAAGTGCACTGACGACGGAGCCCAGAGCCGAAGCCGGC
ACTGTGAGGAGCTCCTCCCAGGGTCCAGCGCCTGTGCTGGAAACAGCAGCCAGAGCCGCCCCTGCCCCTACAG
CGAGATTCCCGTCATCCTGCCAGCCTCCAGCATGGAGGAGGCCACCGGCTGTGCAGGGTTCAATCTCATCCAC
TTGGTGGCCACGGGCATCTCCTGCTTCTTGGGCTCTGGGCTCCTGACCCTAGCAGTGTACCTGTCTTGCCAGC
ACTGCCAGCGTCAGTCCCAGGAGTCCACACTGGTCCATCCTGCCACCCCCAACCATTTGCACTACAAGGGCGG
AGGCACCCCGAAGAATGAAAAGTACACACCCATGGAATTCAAGACCCTGAACAAGAATAACTTGATCCCTGAT
GACAGAGCCAACTTCTACCCATTGCAGCAGACCAATGTGTACACGACTACTTACTACCCAAGCCCCCTGAACA
AACACAGCTTCCGGCCCGAGGCCTCACCTGGACAACGGTGCTTCCCCAACAGCTGATACCGCCGTCCTGGGGA
CTTGGGCTTCTTGCCTTCATAAGGCACAGAGCAGATGGAGATGGGACAGTGGAGCCAGTTTGGTTTTCTCCCT
CTGCACTAGGCCAAGAACTTGCTGCCTTGCCTGTGGGGGGTCCCATCCGGCTTCAGAGAGCTCTGGCTGGCAT
TGACCATGGGGGAAAGGGCTGGTTTCAGGCTGACATATGGCCGCAGGTCCAGTTCAGCCCAGGTCTCTCATGG
TTATCTTCCAACCCACTGTCACGCTGACACTATGCTGCCATGCCTGGGCTGTGGACCTACTGGGCATTTGAGG
AACTGGAGAATGGAGATGGCAAGAGGGCAGGCTTTTAAGTTTGGGTTGGAGACAACTTCCTGTGGCCCCCACA
AGCTGAGTCTGGCCTTCTCCAGCTGGCCCCAAAAAAGGCCTTTGCTACATCCTGATTATCTCTGAAAGTAATC
AATCAAGTGGCTCCAGTAGCTCTGGATTTTCTGCCAGGGCTGGGCCATTGTGGTGCTGCCCCAGTATGACATG
GGACCAAGGCCAGCGCAGGTTATCCACCTCTGCCTGGAAGTCTATACTCTACCCAGGGCATCCCTCTGGTCAG
AGGCAGTGAGTACTGGGAACTGGAGGCTGACCTGTGCTTAGAAGTCCTTTAATCTGGGCTGGTACAGGCCTCA
GCCTTGCCCTCAATGCACGAAAGGTGGCCCAGGAGAGAGGATCAATGCCACAGGAGGCAGAAGTCTGGCCTCT
GTGCCTCTATGGAGACTATCTTCCAGTTGCTGCTCAACAGAGTTGTTGGCTGAGACCTGCTTGGGAGTCTCTG
CTGGCCCTTCATCTGTTCAGGAACACACACACACACACACTCACACACGCACACACAATCACAATTTGCTACA
GCAACAAAAAAGACATTGGGCTGTGGCATTATTAATTAAAGATGATATCCAGTCTCC

| | | | |
|---|---|---|---|
| NOV2a, CG106951-01 | | | |
| Protein Sequence | SEQ ID NO: 6 | 1352 aa | MW at 145674.1kD |

MPAGEGASAHRAGHHTRQARGGSRPSSRGMQGAPSRSSARLEAGGCSARRGRSAPAPSSFSLPLPSFSPFACN
SSPTAPSLLLLPRSPPPCSLRAPGRELVGARGLVPEPSSAEPGGSAAHPAAAGSPSAAGAGPGGDCTGALRAG
GRSCAAAPFPDRPPAHLVSSRRSAPPGSREPRGTGHLHPPLGVSGSSWCLACVSWMPCGFSPSPVAHHLVPGP
PDTPAQQLRCGWTVGGWLLSLVRGLLPCLPPGARTAEGPIMVLAGPLAVSLLLPSLTLLVSHLSSSQDVSSEP
SSEQQLCALSKHPTVAFEDLQPWVSNFTYPGARDFSQLALDPSGNQLIVGARNYLFRLSLANVSLLQATEWAS
SEDTRRSCQSKGKTEEECQNYVRVLIVAGRKVFMCGTNAFSPMCTSRQVGNLSRTTEKINGVARCPYDPRHNS
TAVISSQGELYAATVIDFSGRDPAIYRSLGSGPPLRTAQYNSKWLNEPNFVAAYDIGLFAYFFLRENAVEHDC
GRTVYSRVARVCKNDVGGRFLLEDTWTTFMKARLNCSRPGEVPFYYNELQSAFHLPEQDLIYGVFTTNVNSIA
ASAVCAFNLSAISQAFNGPFRYQENPRAAWLPIANPIPNFQCGTLPETGPNENLTERSLQDAQRLFLMSEAVQ
PVTPEPCVTQDSVRFSHLVVDLVQAKDTLYHVLYIGTESGTILKALSTASRSLHGCYLEELHVLPPGRREPLR
SLRILHSARALFVGLRDGVLRVPLERCAAYRSQGACLGARDPYCGWDGKQQRCSTLEDSSNMSLWTQNITACP
VRNVTRDGGFGPWSPWQPCEHLDGDNSGSCLCRARSCDSPRPRCGGLDCLGPAIHIANCSRNGAWTPWSSWAL
CSTSCGIGFQVRQRSCSNPAPRHGGRICVGKSREERFCNENTPCPVPIFWASWGSWSKCSSNCGGGMQSRRRA
CENGNSCLGCGVEFKTCNPEGCPEVRRNTPWTPWLPVNVTQGGARQEQRFRFTCRAPLADPHGLQFGRRRTET
RTCPADGSGSCDTDALVEDLLRSGSTSPHTVSGGWAAWGPWSSCSRDCELGFRVRKRTCTNPEPRNGGLPCVG
DAAEYQDCNPQACPVRGAWSCWTSWSPCSASCGGGHYQRTRSCTSPAPSPGEDICLGLHTEEALCATQACPEG
WSPWSEWSKCTDDGAQSRSRHCEELLPGSSACAGNSSQSRPCPYSEIPVILPASSMEEATGCAGFNLIHLVAT
GISCFLGSGLLTLAVYLSCQHCQRQSQESTLVHPATPNHLHYKGGGTPKNEKYTPMEFKTLNKNNLIPDDRAN
FYPLQQTNVYTTTYYPSPLNKHSFRPEASPGQRCFPNS

| | | |
|---|---|---|
| NOV2b, CG106951-04 | SEQ ID NO: 7 | 3631 bp |
| DNA Sequence | ORF Start: ATG at 154 | ORF Stop: TGA at 3544 |

GCGGCCGCCCCATTCCCAGACCGGCCGCCAGCCCATCTGGTTAGCTCCCGCCGCTCCGCGCCGCCCGGGAGTC
GGGAGCCGCGGGGAACCGGGCACCTGCACCCGCCTCTGGGAGTGAGTGGTTCCAGCTGGTGCCTGGCCTGTGT
CTCTTGGATGCCCTGTGGCTTCAGTCCGTCTCCTGTTGCCCACCACCTCGTCCCTGGGCCGCCTGATACCCCA
GCCCAACAGCTAAGGTGTGGATGGACAGTAGGGGGCTGGCTTCTCTCACTGGTCAGGGGTCTTCTCCCCTGTC
TGCCTCCCGGAGCTAGGACTGCAGAGGGGCCTATCATGGTGCTTGCAGGCCCCCTGGCTGTCTCGCTGTTGCT
GCCCAGCCTCACACTGCTGGTGTCCCACCTCTCCAGCTCCCAGGATGTCTCCAGTGAGCCCAGCAGTGAGCAG
CAGCTGTGCGCCCTTAGCAAGCACCCCACCGTGGCCTTTGAAGACCTGCAGCCGTGGGTCTCTAACTTCACCT
ACCCTGGAGCCCGGGATTTCTCCCAGCTGGCTTTGGACCCCTCCGGGAACCAGCTCATCGTGGGAGCCAGGAA
CTACCTCTTCAGACTCAGCCTTGCCAATGTCTCTCTTCTTCAGGCCACAGAGTGGGCCTCCAGTGAGGACACG
CGCCGCTCCTGCCAAAGCAAAGGGAAGACTGAGGAGGAGTGTCAGAACTACGTGCGAGTCCTGATCGTCGCCG
GCCGGAAGGTGTTCATGTGTGGAACCAATGCCTTTTCCCCCATGTGCACCAGCAGACAGGTGGGGAACCTCAG
CCGGACTACTGAGAAGATCAATGGTGTGGCCCGCTGCCCCTATGACCCACGCCACAACTCCACAGCTGTCATC
TCCTCCCAGGGGGAGCTCTATGCAGCCACGGTCATCGACTTCTCAGGTCGGGACCCTGCCATCTACCGCAGCC
TGGGCAGTGGGCCACCGCTTCGCACTGCCCAATATAACTCCAAGTGGCTTAATGAGCCAAACTTCGTGGCAGC
CTATGATATTGGGCTGTTTGCATACTTCTTCCTGCGGGAGAACGCAGTGGAGCACGACTGTGGACGCACCGTG
TACTCTCGCGTGGCCCGCGTGTGCAAGAATGACGTGGGGGGCCGATTCCTGCTGGAGGACACATGGACCACAT
TCATGAAGGCCCGGCTCAACTGCTCCCGCCCGGGCGAGGTCCCCTTCTACTATAACGAGCTGCAGAGTGCCTT
CCACTTGCCAGAGCAGGACCTCATCTATGGAGTTTTCACAACCAACGTAAACAGCATCGCGGCTTCTGCTGTC
TGCGCCTTCAACCTCAGTGCTATCTCCCAGGCTTTCAATGGCCCATTTCGCTACCAGGAGAACCCCAGGGCTG
CCTGGCTCCCCATAGCCAACCCCATCCCCAATTTCCAGTGTGGCACCCTGCCTGAGACCGGTCCCAACGAGAA
CCTGACGGAGCGCAGCCTGCAGGACGCGCAGCGCCTCTTCCTGATGAGCGAGGCCGTGCAGCCGGTGACACCC
GAGCCCTGTGTCACCCAGGACAGCGTGCGCTTCTCACACCTCGTGGTGGACCTGGTGCAGGCTAAAGACACGC
TCTACCATGTACTCTACATTGGCACCGAGTCGGGCACCATCCTGAAGGCGCTGTCCACGGCGAGCCGCAGCCT
CCACGGCTGCTACCTGGAGGAGCTGCACGTGCTGCCCCCCGGGCGCCGCGAGCCCCTGCGCAGCCTGCGCATC
CTGCACAGCGCCCGCGCGCTCTTCGTGGGGCTGAGAGACGGCGTCCTGCGGGTCCCACTGGAGAGGTGCGCCG
CCTACCGCAGCCAGGGGGCATGCCTGGGGGCCCGGGACCCGTACTGTGGCTGGGACGGGAAGCAGCAACGTTG
CAGCACACTCGAGGACAGCTCCAACATGAGCCTCTGGACCCAGAACATCACCGCCTGTCCTGTGCGGAATGTG
ACACGGGATGGGGGCTTCGGCCCATGGTCACCATGGCAACCATGTGAGCACTTGGATGGGGACAACTCAGGCT
CTTGCCTGTGTCGAGCTCGATCCTGTGATTCCCCTCGACCCCGCTGTGGGGGCCTTGACTGCCTGGGGCCAGC
CATCCACATCGCCAACTGCTCCAGGAATGGGGCGTGGACCCCGTGGTCATCGTGGGCGCTGTGCAGCACGTCC
TGTGGCATCGGCTTCCAGGTCCGCCAGCGAAGTTGCAGCAACCCTGCTCCCCGCCACGGGGGCCGCATCTGCG
TGGGCAAGAGCCGGGAGGAACGGTTCTGTAATGAGAACACGCCTTGCCCGGTGCCCATCTTCTGGGCTTCCTG
GGGCTCCTGGAGCAAGTGCAGCAGCAACTGTGGAGGGGGCATGCAGTCGCGGCGTCGGGCCTGCGAGAACGGC
AACTCCTGCCTGGGCTGCGGCGTGGAGTTCAAGACGTGCAACCCCGAGGGCTGCCCCGAAGTGCGGCGCAACA
CCCCCTGGACGCCGTGGCTGCCCGTGAACGTGACGCAGGGCGGGGCACGGCAGGAGCAGCGGTTCCGCTTCAC
CTGCCGCGCGCCCCTTGCAGACCCGCACGGCCTGCAGTTCGGCAGGAGAAGGACCGAGACGAGGACCTGTCCC
GCGGACGGCTCCGGCTCCTGCGACACCGACGCCCTGGTGGAGGACCTCCTGCGCAGCGGGAGCACCTCCCCGC
ACACGGTGAGCGGGGGCTGGGCCGCCTGGGGCCCGTGGTCGTCCTGCTCCCGGGACTGCGAGCTGGGCTTCCG
CGTCCGCAAGAGAACGTGCACTAACCCGGAGCCCCGCAACGGGGGCCTGCCCTGCGTGGGCGATGCTGCCGAG
TACCAGGACTGCAACCCCCAGGCTTGCCCAGTTCGGGGTGCTTGGTCCTGCTGGACCTCATGGTCTCCATGCT
CAGCTTCCTGTGGTGGGGGTCACTATCAACGCACCCGTTCCTGCACCAGCCCCGCACCCTCCCCAGAAGGCTG
GTCGCCCTGGTCTGAGTGGAGTAAGTGCACTGACGACGGAGCCCAGAGCCGAAGCCGGCACTGTGAGGAGCTC
CTCCCAGGGTCCAGCGCCTGTGCTGGAAACAGCAGCCAGAGCCGCCCCTGCCCCTACAGCGAGATTCCCGTCA
TCCTGCCAGCCTCCAGCATGGAGGAGGCCACCGGCTGTGCAGGGTTCAATCTCATCCACTTGGTGGCCACGGG
CATCTCCTGCTTCTTGGGCTCTGGGCTCCTGACCCTAGCAGTGTACCTGTCTTGCCAGCACTGCCAGCGTCAG
TCCCAGGAGTCCACACTGGTCCATCCTGCCACCCCCAACCATTTGCACTACAAGGGCGGAGGCACCCCGAAGA
ATGAAAAGTACACACCCATGGAATTCAAGACCCTGAACAAGAATAACTTGATCCCTGATGACAGAGCCAACTT
CTACCCATTGCAGCAGACCAATGTGTACACGACTACTTACTACCCAAGCCCCCTGAACAAACACAGCTTCCGG
CCCGAGGCCTCACCTGGACAACGGTGCTTCCCCAACAGCTGATACCGCCGTCCTGGGGACTTGGGCTTCTTGC
CTTCATAAGGCACAGAGCAGATGGAGATGGGACAGTGGAGCCAGTTTGGTTTCT

| | | | |
|---|---|---|---|
| NOV2b, CG106951-04 | | | |
| Protein Sequence | SEQ ID NO: 8 | 1130 aa | MW at 123700.9kD |

MPCGFSPSPVAHHLVPGPPDTPAQQLRCGWTVGGWLLSLVRGLLPCLPPGARTAEGPIMVLAGPLAVSLLLPS
LTLLVSHLSSSQDVSSEPSSEQQLCALSKHPTVAFEDLQPWVSNFTYPGARDFSQLALDPSGNQLIVGARNYL
FRLSLANVSLLQATEWASSEDTRRSCQSKGKTEEECQNYVRVLIVAGRKVFMCGTNAFSPMCTSRQVGNLSRT
TEKINGVARCPYDPRHNSTAVISSQGELYAATVIDFSGRDPAIYRSLGSGPPLRTAQYNSKWLNEPNFVAAYD
IGLFAYFFLRENAVEHDCGRTVYSRVARVCKNDVGGRFLLEDTWTTFMKARLNCSRPGEVPFYYNELQSAFHL
PEQDLIYGVFTTNVNSIAASAVCAFNLSAISQAFNGPFRYQENPRAAWLPIANPIPNFQCGTLPETGPNENLT
ERSLQDAQRLFLMSEAVQPVTPEPCVTQDSVRFSHLVVDLVQAKDTLYHVLYIGTESGTILKALSTASRSLHG
CYLEELHVLPPGRREPLRSLRILHSARALFVGLRDGVLRVPLERCAAYRSQGACLGARDPYCGWDGKQQRCST
LEDSSNMSLWTQNITACPVRNVTRDGGFGPWSPWQPCEHLDGDNSGSCLCRARSCDSPRPRCGGLDCLGPAIH
IANCSRNGAWTPWSSWALCSTSCGIGFQVRQRSCSNPAPRHGGRICVGKSREERFCNENTPCPVPIFWASWGS
WSKCSSNCGGGMQSRRRACENGNSCLGCGVEFKTCNPEGCPEVRRNTPWTPWLPVNVTQGGARQEQRFRFTCR
APLADPHGLQFGRRRTETRTCPADGSGSCDTDALVEDLLRSGSTSPHTVSGGWAAWGPWSSCSRDCELGFRVR
KRTCTNPEPRNGGLPCVGDAAEYQDCNPQACPVRGAWSCWTSWSPCSASCGGGHYQRTRSCTSPAPSPEGWSP
WSEWSKCTDDGAQSRSRHCEELLPGSSACAGNSSQSRPCPYSEIPVILPASSMEEATGCAGFNLIHLVATGIS
CFLGSGLLTLAVYLSCQHCQRQSQESTLVHPATPNHLHYKGGGTPKNEKYTPMEFKTLNKNNLIPDDRANFYP
LQQTNVYTTTYYPSPLNKHSFRPEASPGQRCFPNS

| | | |
|---|---|---|
| NOV2c, 209829549 | SEQ ID NO: 9 | 1203 bp |
| DNA Sequence | ORF Start: at 1 | ORF Stop: end of sequence |

GGATCCGGCCCATGGTCACCATGGCAACCATGTGAGCACTTGGATGGGGACAACTCAGGCTCTTGCCTGTGTC
GAGCTCGATCCTGTGATTCCCCTCGACCCCGCTGTGGGGGCCTTGACTGCCTGGGGCCAGCCATCCACATCGC
CAACTGCTCCAGGAATGGGGCGTGGACCCCGTGGTCATCGTGGGCGCTGTGCAGCACGTCCTGTGGCATCGGC
TTCCAGGTCCGCCAGCGAAGTTGCAGCAACCCTGCTCCCCGCCACGGGGGCCGCATCTGCGTGGGCAAGAGCC
GGGAGGAACGGTTCTGTAATGAGAACACGCCTTGCCCGGTGCCCATCTTCTGGGCTTCCTGGGGCTCCTGGAG
CAAGTGCAGCAGCAACTGTGGAGGGGGCATGCGGTCGCGGCGTCGGGCCTGCGAGAACGGCAACTCCTGCCTG
GGCTGCGGCGTGGAGTTCAAGACGTGCAACCCCGAGGGCTGCCCCGAAGTGCGGCGCAACACCCCCTGGACGC
CGTGGCTGCCCGTGAACGTGACGCAGGGCGGGGCACGGCAGGAGCAGCGGTTCCGCTTCACCTGCCGCGCGCC
CCTTGCAGACCCGCACGGCCTGCAGTTCGGCAGGAGAAGGACCGAGACGAGGACCTGTCCCGCGGACGGCTCC
GGCTCCTGCGACACCGACGCCCTGGTGGAGGACCTCCTGCGCAGCGGGAGCACCTCCCCGCACACGGTGAGCG
GGGGCTGGGCCGCCTGGGGCCCGTGGTCGTCCTGCTCCCGGGACTGCGAGCTGGGCTTCCGCGTCCGCAAGAG
AACGTGCACTAACCCGGAGCCCCGCAACGGGGGCCTGCCCTGCGTGGGCGATGCTGCCGAGTACCAGGACTGC
AACCCCCAGGCTTGCCCAGTTCGGGGTGCTTGGTCCTGCTGGACCTCATGGTCTCCATGCTCAGCTTCCTGTG
GTGGGGGTCACTATCAACGCACCCGTTCCTGCACCAGCCCCGCACCCTCCCCAGGTGAGGACATCTGTCTCGG
GCTGCACACGGAGGAGGCACTATGTGCCACACAGGCCTGCCCAGAAGGCTGGTCGCCCTGGTCTGAGTGGAGT
AAGTGCACTGACGACGGAGCCCAGAGCCGAAGCCGGCACTGTGAGGAGCTCCTCCCAGGGTCCAGCGCCTGTG
CTGGAAACAGCAGCCAGAGCCGCCCCTGCGTCGAC

| | | | |
|---|---|---|---|
| NOV2c, 209829549 | | | |
| Protein Sequence | SEQ ID NO: 10 | 401 aa | MW at 43284.5kD |

GSGPWSPWQPCEHLDGDNSGSCLCRARSCDSPRPRCGGLDCLGPAIHIANCSRNGAWTPWSSWALCSTSCGIG
FQVRQRSCSNPAPRHGGRICVGKSREERFCNENTPCPVPIFWASWGSWSKCSSNCGGGMRSRRRACENGNSCL
GCGVEFKTCNPEGCPEVRRNTPWTPWLPVNVTQGGARQEQRFRFTCRAPLADPHGLQFGRRRTETRTCPADGS
GSCDTDALVEDLLRSGSTSPHTVSGGWAAWGPWSSCSRDCELGFRVRKRTCTNPEPRNGGLPCVGDAAEYQDC
NPQACPVRGAWSCWTSWSPCSASCGGGHYQRTRSCTSPAPSPGEDICLGLHTEEALCATQACPEGWSPWSEWS
KCTDDGAQSRSRHCEELLPGSSACAGNSSQSRPCVD

| | | |
|---|---|---|
| NOV2d, 209829553 | SEQ ID NO: 11 | 1203 bp |
| DNA Sequence | ORF Start: at 1 | ORF Stop: end of sequence |

GGATCCGGCCCATGGTCACCATGGCAACCATGTGAGCACTTGGATGGGGACAACTCAGGCTCTTGCCTGTGTC
GAGCTCGATCCTGTGATTCCCCTCGACCCCGCTGTGGGGGCCTTGACTGCCTGGGGCCAGCCATCCACATCGC
CAACTGCTCCAGGAATGGGGCGTGGACCCCGTGGTCATCGTGGGCGCTGTGCAGCACGTCCTGTGGCATCGGC
TTCCAGGTCCGCCAGCGAAGTTGCAGCAACCCTGCTCCCCGCCACGGGGGCCGCATCTGCGTGGGCAAGAGCC
GGGAGGAACGGTTCTGTAATGAGAACACGCCTTGCCCGGTGCCCATCTTCTGGGCTTCCTGGGGCTCCTGGAG
CAAGTGCAGCAGCAACTGTGGAGGGGGCATGCAGTCGCGGCGTCGGGCCTGCGAGAACGGCAACTCCTGCCTG
GGCTGCGGCGTGGAGTTCAAGACGTGCAACCCCGAGGGCTGCCCCGAAGTGCGGCGCAACACCCCCTGGACGC
CGTGGCTGCCCGTGAACGTGACGCAGGGCGGGGCACGGCAGGAGCAGCGGTTCCGCTTCACCTGCCGCGCGCC
CCTTGCAGACCCGCACGGCCTGCAGTTCGGCAGGAGAAGGACCGAGACGAGGACCTGTCCCGCGGACGGCTCC
GGCTCCTGCGACACCGACGCCCTGGTGGAGGACCTCCTGCGCAGCGGGAGCACCTCCCCGCACACGGTGAGCG
GGGGCTGGGCCGCCTGGGGCCCGTGGTCGTCCTGCTCCCGGGACTGCGAGCTGGGCTTCCGCGTCCGCAAGAG
AACGTGCACTAACCCGGAGTCCCGCAACGGGGGCCTGCCCTGCGTGGGCGATGCTGCCGAGTACCAGGACTGC
AACCCCCAGGCTTGCCCAGTTCGGGGTGCTTGGTCCTGCTGGACCTCATGGTCTCCATGCTCAGCTTCCTGTG
GTGGGGGTCACTATCAACGCACCCGTTCCTGCACCAGCCCCGCACCCTCCCCAGGTGAGGACATCTGTCTCGG
GCTGCACACGGAGGAGGCACTATGTGCCACACAGGCCTGCCCAGAAGGCTGGTCGCCCTGGTCTGAGTGGAGT
AAGTGCACTGACGACGGAGCCCAGAGCCGAAGCCGGCACTGTGAGGAGCTCCTCCCAGGGTCCAGCGCCTGTG
CTGGAAACAGCAGCCAGAGCCGCCCCTGCGTCGAC

| | | | |
|---|---|---|---|
| NOV2d, 209829553 | | | |
| Protein Sequence | SEQ ID NO: 12 | 401 aa | MW at 43246.4kD |

GSGPWSPWQPCEHLDGDNSGSCLCRARSCDSPRPRCGGLDCLGPAIHIANCSRNGAWTPWSSWALCSTSCGIG
FQVRQRSCSNPAPRHGGRICVGKSREERFCNENTPCPVPIFWASWGSWSKCSSNCGGGMQSRRRACENGNSCL
GCGVEFKTCNPEGCPEVRRNTPWTPWLPVNVTQGGARQEQRFRFTCRAPLADPHGLQFGRRRTETRTCPADGS
GSCDTDALVEDLLRSGSTSPHTVSGGWAAWGPWSSCSRDCELGFRVRKRTCTNPESRNGGLPCVGDAAEYQDC
NPQACPVRGAWSCWTSWSPCSASCGGGHYQRTRSCTSPAPSPGEDICLGLHTEEALCATQACPEGWSPWSEWS
KCTDDGAQSRSRHCEELLPGSSACAGNSSQSRPCVD

| | | |
|---|---|---|
| NOV2e, 209829642 | SEQ ID NO: 13 | 1203 bp |
| DNA Sequence | ORF Start: at 1 | ORF Stop: end of sequence |

GGATCCGGCCCATGGTCACCATGGCAACCATGTGAGCACTTGGATGGGGACAACTCAGGCTCTTGCCTGTGTC
GAGCTCGATCCTGTGATTCCCCTCGACCCCGCTGTGGGGGCCTTGACTGCCTGGGGCCAGCCATCCACATCGC
CAACTGCTCCAGGAATGGGGCGTGGACCCCGTGGTCATCGTGGGCGCTGTGCAGCACGTCCTGTGGCATCGGC
TTCCAGGTCCGCCAGCGAAGTTGCAGCAACCCTGCTCCCCGCCACGGGGGCCGCATCTGCGTGGGCAAGAGCC
GGGAGGAACGGTTCTGTAATGAGAACACGCCTTGCCCGGTGCCCATCTTCTGGGCTTCCTGGGGCTCCTGGAG
CAAGTGCAGCAGCAACTGTGGAGGGGGCATGCAGTCGCGGCGTCGGGCCTGCGAGAACGGCAACTCCTGCCTG
GGCTGCGGCGTGGAGTTCAAGACGTGCAACCCCGAGGGCTGCCCCGAAGTGCGGCGCAACACCCCCTGGACGC
CGTGGCTGCCCGTGAACGTGACGCAGGGCGGGGCACGGCAGGAGCAGCGGTTCCGCTTCACCTGCCGCGCGCC
CCTTGCAGACCCGCACGGCCTGCAGTTCGGCAGGAGAAGGACCGAGACGAGGACCTGTCCCGCGGACGGCTCC
GGCTCCTGCGACACCGACGCCCTGGTGGAGGACCTCCTGCGCAGCGGGAGCACCTCCCCGCACACGGTGAGCG
GGGGCTGGGCCGCCTGGGGCCCGTGGTCGTCCTGCTCCCGGGACTGCGAGCTGGGCTTCCGCGTCCGCAAGAG
AACGTGCACTAACCCGGAGCCCCGCAACGGGGGCCTGCCCTGCGTGGGCCATGCTGCCGAGTACCAGGACTGC
AACCCCCAGGCTTGCCCAGTTCGGGGTGCTTGGTCCTGCTGGACCTCATGGTCTCCATGCTCAGCTTCCTGTG
GTGGGGGTCACTATCAACGCACCCGTTCCTGCACCAGCCCCGCACCCTCCCCAGGTGAGGACATCTGTCTCGG
GCTGCACACGGAGGAGGCACTATGTGCCACACAGGCCTGCCCAGAAGGCTGGTCGCCCTGGTCTGAGTGGAGT
AAGTGCACTGACGACGGAGCCCAGAGCCGAAGCCGGCACTGTGAGGAGCTCCTCCCAGGGTCCAGCGCCTGTG
CTGGAAACAGCAGCCAGAGCCGCCCCTGCGTCGAC

| | | | |
|---|---|---|---|
| NOV2e, 209829642 | | | |
| Protein Sequence | SEQ ID NO: 14 | 401 aa | MW at 43256.4kD |

GSGPWSPWQPCEHLDGDNSGSCLCRARSCDSPRPRCGGLDCLGPAIHIANCSRNGAWTPWSSWALCSTSCGIG
FQVRQRSCSNPAPRHGGRICVGKSREERFCNENTPCPVPIFWASWGSWSKCSSNCGGGMQSRRRACENGNSCL
GCGVEFKTCNPEGCPEVRRNTPWTPWLPVNVTQGGARQEQRFRFTCRAPLADPHGLQFGRRRTETRTCPADGS
GSCDTDALVEDLLRSGSTSPHTVSGGWAAWGPWSSCSRDCELGFRVRKRTCTNPEPRNGGLPCVGDAAEYQDC
NPQACPVRGAWSCWTSWSPCSASCGGGHYQRTRSCTSPAPSPGEDICLGLHTEEALCATQACPEGWSPWSEWS
KCTDDGAQSRSRHCEELLPGSSACAGNSSQSRPCVD

| | | |
|---|---|---|
| NOV2f, 209829670 | SEQ ID NO: 15 | 1203 bp |
| DNA Sequence | ORF Start: at 1 | ORF Stop: end of sequence |

GGATCCGCCCCATGGTCACCATGGCAACCATGTGAGCACTTGGATGGGGACAACTCAGGCTCTTGCCTGTGTC
GAGCTCGATCCTGTGATTCCCCTCGACCCCGCTGTGGGGGCCTTGACTGCCTGGGGCCAACCATCCACATCGC
CAACTGCTCCAGGAATGGGGCGTGGACCCCGTGGTCATCGTGGGCGCTGTGCAGCACGTCCTGTGGCATCGGC
TTCCAGGTCCGCCAGCGAAGTTGCAGCAACCCTGCTCCCCGCCACGGGGGCCGCATCTGCGTGGGCAAGAGCC
GGGAGGAACGGTTCTGTAATGAGAACACGCCTTGCCCGGTGCCCATCTTCTGGGCTTCCTGGGGCTCCTGGAG
CAAGTGCGGCAGCAACTGTGGAGGGGGCATGCAGTCGCGGCGTCGGGCCTGCGAGAACGGCAACTCCTGCCTG
GGCTGCGGCGTGGAGTTCAAGACGTGCAACCCCGAGGGCTGCCCCGAAGTGCGGCGCAACACCCCCTGGACGC
CGTGGCTGCCCGTGAACGTGACGCAGGGCGGGGCACGGCAGGAGCAGCGGTTCCGCTTCACCTGCCGCGCGCC
CCTTGCAGACCCGCACGGCCTGCAGTTCGGCAGGAGAAGGACCGAGACGAGGACCTGTCCCGCGGACGGCTCC
GGCTCCTGCGACACCGACGCCCTGGTGGAGGTCCTCCTGCGCAGCGGGAGCACCTCCCCGCACACGGTGAGCG
GGGGCTGGGCCGCCTGGGGCCCGTGGTCGTCCTGCTCCCGGGACTGCGAGCTGGGCTTCCGCGTCCGCAAGAG
AACGTGCACTAACCCGGAGCCCCGCAACGGGGGCCTGCCCTGCGTGGGCGATGCTGCCGAGTACCAGGACTGC
AACCCCCAGGCTTGCCCAGTTCGGGGTGCTTGGTCCTGCTGGACCTCATGGTCTCCATGCTCAGCTTCCTGTG
GTGGGGGTCACTATCAACGCACCCGTTCCTGCACCAGCCCCGCACCCTCCCCAGGTGAGGACATCTGTCTCGG
GCTGCACACGGAGGAGGCACTATGTGCCACACAGGCCTGCCCAGAAGGCTGGTCGCCCTGGTCTGAGTGGAGT
AAGTGCACTGACGACGGAGCCCAGAGCCGAAGCCGGCACTGTGAGGAGCTCCTCCCAGGGTCCAGCGCCTGTG
CTGGAAACAGCAGCCAGAGCCGCCCCTGCGTCGAC

| | | | |
|---|---|---|---|
| NOV2f, 209829670 | | | |
| Protein Sequence | SEQ ID NO: 16 | 401 aa | MW at 43240.5kD |

GSGPWSPWQPCEHLDGDNSGSCLCRARSCDSPRPRCGGLDCLGPTIHIANCSRNGAWTPWSSWALCSTSCGIG
FQVRQRSCSNPAPRHGGRICVGKSREERFCNENTPCPVPIFWASWGSWSKCGSNCGGGMQSRRRACENGNSCL
GCGVEFKTCNPEGCPEVRRNTPWTPWLPVNVTQGGARQEQRFRFTCRAPLADPHGLQFGRRRTETRTCPADGS
GSCDTDALVEVLLRSGSTSPHTVSGGWAAWGPWSSCSRDCELGFRVRKRTCTNPEPRNGGLPCVGDAAEYQDC
NPQACPVRGAWSCWTSWSPCSASCGGGHYQRTRSCTSPAPSPGEDICLGLHTEEALCATQACPEGWSPWSEWS
KCTDDGAQSRSRHCEELLPGSSACAGNSSQSRPCVD

| | | |
|---|---|---|
| NOV2g, CG106951-02 | SEQ ID NO: 17 | 4233 bp |
| DNA Sequence | ORF Start: ATG at 2 | ORF Stop: TGA at 3281 |

CATGGTGCTTGCAGGCCCCCTGGCTGTCTCGCTGTTGCTGCCCAGCCTCACACTGCTGGTGTCCCACCTCTCC
AGCTCCCAGGATGTCTCCAGTGAGCCCAGCAGTGAGCAGCAGCTGTGCGCCCTTAGCAAGCACCCCACCGTGG
CCTTTGAAGACCTGCAGCCGTGGGTCTCTAACTTCACCTACCCTGGAGCCCGGGATTTCTCCCAGCTGGCTTT
GGACCCCTCCGGGAACCAGCTCATCGTGGGAGCCAGGAACTACCTCTTCAGACTCAGCCTTGCCAATGTCTCT
CTTCTTCAGGCCACAGAGTGGGCCTCCAGTGAGGACACGCGCCGCTCCTGCCAAAGCAAAGGGAAGACTGAGG
AGGAGTGTCAGAACTACGTGCGAGTCCTGATCGTCGCCGGCCGGAAGGTGTTCATGTGTGGAACCAATGCCTT
TTCCCCCATGTGCACCAGCAGACAGGTGGGGAACCTCAGCCGGACTACTGAGAAGATCAATGGTGTGGCCCGC
TGCCCCTATGACCCACGCCACAACTCCACAGCTGTCATCTCCTCCCAGGGGGAGCTCTATGCAGCCACGGTCA
TCGACTTCTCAGGTCGGGACCCTGCCATCTACCGCAGCCTGGGCAGTGGGCCACCGCTTCGCACTGCCCAATA
TAACTCCAAGTGGCTTAATGAGCCAAACTTCGTGGCAGCCTATGATATTGGGCTGTTTGCATACTTCTTCCTG
CGGGAGAACGCAGTGGAGCACGACTGTGGACGCACCGTGTACTCTCGCGTGGCCCGCGTGTGCAAGAATGACG
TGGGGGGCCGATTCCTGCTGGAGGACACATGGACCACATTCATGAAGGCCCGGCTCAACTGCTCCCGCCCGGG
CGAGGTCCCCTTCTACTATAACGAGCTGCAGAGTGCCTTCCACTTGCCAGAGCAGGACCTCATCTATGGAGTT
TTCACAACCAACGTAAACAGCATCGCGGCTTCTGCTGTCTGCGCCTTCAACCTCAGTGCTATCTCCCAGGCTT
TCAATGGCCCATTTCGCTACCAGGAGAACCCCAGGGCTGCCTGGCTCCCCATAGCCAACCCCATCCCCAATTT
CCAGTGTGGCACCCTGCCTGAGACCGGTCCCAACGAGAACCTGACGGAGCGCAGCCTGCAGGACGCGCAGCGC
CTCTTCCTGATGAGCGAGGCCGTGCAGCCGGTGACACCCGAGCCCTGTGTCACCCAGGACAGCGTGCGCTTCT
CACACCTCGTGGTGGACCTGGTGCAGGCTAAAGACACGCTCTACCATGTACTCTACATTGGCACCGAGTCGGG
CACCATCCTGAAGGCGCTGTCCACGGCGAGCCGCAGCCTCCACGGCTGCTACCTGGAGGAGCTGCACGTGCTC
CCCCCCGGGCGCCGCGAGCCCCTGCGCAGCCTGCGCATCCTGCACAGCGCCCGCGCGCTCTTCGTGGGGCTGA
GAGACGGCGTCCTGCGGGTCCCACTGGAGAGGTGCGCCGCCTACCGCAGCCAGGGGGCATGCCTGGGGGCCCG
GGACCCGTACTGTGGCTGGGACGGGAAGCAGCAACGTTGCAGCACACTCGAGGACAGCTCCAACATGAGCCTC
TGGACCCAGAACATCACCGCCTGTCCTGTGCGGAATGTGACACGGGATGGGGGCTTCGGCCCATGGTCACCAT
GGCAACCATGTGAGCACTTGGATGGGGACAACTCAGGCTCTTGCCTGTGTCGAGCTCGATCCTGTGATTCCCC
TCGACCCCGCTGTGGGGGCCTTGACTGCCTGGGGCCAGCCATCCACATCGCCAACTGCTCCAGGAATGGGGCG
TGGACCCCGTGGTCATCGTGGGCGCTGTGCAGCACGTCCTGTGGCATCGGCTTCCAGGTCCGCCAGCGAAGTT
GCAGCAACCCTGCTCCCCGCCACGGGGGCCGCATCTGCGTGGGCAAGAGCCGGGAGGAACGGTTCTGTAATGA
GAACACGCCTTGCCCGGTGCCCATCTTCTGGGCTTCCTGGGGCTCCTGGAGCAAGTGCAGCAGCAACTGTGGA
GGGGGCATGCAGTCGCGGCGTCGGGCCTGCGAGAACGGCAACTCCTGCCTGGGCTGCGGCGTGGAGTTCAAGA
CGTGCAACCCCGAGGGCTGCCCCGAAGTGCGGCGCAACACCCCCTGGACGCCGTGGCTGCCCGTGAACGTGAC
GCAGGGCGGGGCACGGCAGGAGCAGCGGTTCCGCTTCACCTGCCGCGCGCCCCTTGCAGACCCGCACGGCCTG
CAGTTCGGCAGGAGAAGGACCGAGACGAGGACCTGTCCCGCGGACGGCTCCGGCTCCTGCGACACCGACGCCC
TGGTGGAGGACCTCCTGCGCAGCGGGAGCACCTCCCCGCACACGGTGAGCGGGGGCTGGGCCGCCTGGGGCCC
GTGGTCGTCCTGCTCCCGGGACTGCGAGCTGGGCTTCCGCGTCCGCAAGAGAACGTGCACTAACCCGGAGCCC
CGCAACGGGGGCCTGCCCTGCGTGGGCGATGCTGCCGAGTACCAGGACTGCAACCCCCAGGCTTGCCCAGTTC
GGGGTGCTTGGTCCTGCTGGACCTCATGGTCTCCATGCTCAGCTTCCTGTGGTGGGGGTCACTATCAACGCAC
CCGTTCCTGCACCAGCCCCGCACCCTCCCCAGGTGAGGACATCTGTCTCGGGCTGCACACGGAGGAGGCACTA
TGTGCCACACAGGCCTGCCCAGAAGGCTGGTCGCCCTGGTCTGAGTGGAGTAAGTGCACTGACGACGGAGCCC
AGAGCCGAAGCCGGCACTGTGAGGAGCTCCTCCCAGGGTCCAGCGCCTGTGCTGGAAACAGCAGCCAGAGCCG
CCCCTGCCCCTACAGCGAGATTCCCGTCATCCTGCCAGCCTCCAGCATGGAGGAGGCCACCGGCTGTGCAGGG
TTCAATCTCATCCACTTGGTGGCCACGGGCATCTCCTGCTTCTTGGGCTCTGGGCTCCTGACCCTAGCAGTGT
ACCTGTCTTGCCAGCACTGCCAGCGTCAGTCCCAGGAGTCCACACTGGTCCATCCTGCCACCCCCAACCATTT
GCACTACAAGGGCGGAGGCACCCCGAAGAATGAAAAGTACACACCCATGGAATTCAAGACCCTGAACAAGAAT
AACTTGATCCCTGATGACAGAGCCAACTTCTACCCATTGCAGCAGACCAATGTGTACACGACTACTTACTACC
CAAGCCCCCTGAACAAACACAGCTTCCGGCCCGAGGCCTCACCTGGACAACGGTGCTTCCCCAACAGCTGATA
CCGCCGTCCTGGGGACTTGGGCTTCTTGCCTTCATAAGGCACAGAGCAGATGGAGATGGGACAGTGGAGCCAG
TTTGGTTTTCTCCCTCTGCACTAGGCCAAGAACTTGCTGCCTTGCCTGTGGGGGGTCCCATCCGGCTTCAGAG
AGCTCTGGCTGGCATTGACCATGGGGGAAAGGGCTGGTTTCAGGCTGACATATGGCCGCAGGTCCACTTCAGC
CCAGGTCTCTCATGGTTATCTTCCAACCCACTGTCACGCTGACACTATGCTGCCATGCCTGGGCTGTGGACCT
ACTGGGCATTTGAGGAACTGGAGAATGGAGATGGCAAGAGGGCAGGCTTTTAAGTTTGGGTTGGAGACAACTT
CCTGTGGCCCCCACAAGCTGAGTCTGGCCTTCTCCAGCTGGCCCCAAAAAAGGCCTTTGCTACATCCTGATTA
TCTCTGAAAGTAATCAATCAAGTGGCTCCAGTAGCTCTGGATTTTCTGCCAGGGCTGGGCCATTGTGGTGCTG
CCCCAGTATGACATGGGACCAAGGCCAGCGCAGGTTATCCACCTCTGCCTGGAAGTCTATACTCTACCCAGGG
CATCCCTCTGGTCAGAGGCAGTGAGTACTGGGAACTGGAGGCTGACCTGTGCTTAGAAGTCCTTTAATCTGGG
CTGGTACAGGCCTCAGCCTTGCCCTCAATGCACGAAAGGTGGCCCAGGAGAGAGGATCAATGCCACAGGAGGC
AGAAGTCTGGCCTCTGTGCCTCTATGGAGACTATCTTCCAGTTGCTGCTCAACAGAGTTGTTGGCTGAGACCT
GCTTGGGAGTCTCTGCTGGCCCTTCATCTGTTCAGGAACACACACACACACACACTCACACACGCACACACAA
TCACAATTTGCTACAGCAACAAAAAAGACATTGGGCTGTGGCATTATTAATTAAAGATGATATCCAGTCTCC

| | | | |
|---|---|---|---|
| NOV2g, CG106951-02 | | | |
| Protein Sequence | SEQ ID NO: 18 | 1093 aa | MW at 119865.3kD |

MVLAGPLAVSLLLPSLTLLVSHLSSSQDVSSEPSSEQQLCALSKHPTVAFEDLQPWVSNFTYPGARDFSQLAL
DPSGNQLIVGARNYLFRLSLANVSLLQATEWASSEDTRRSCQSKGKTEEECQNYVRVLIVAGRKVFMCGTNAF
SPMCTSRQVGNLSRTTEKINGVARCPYDPRHNSTAVISSQGELYAATVIDFSGRDPAIYRSLGSGPPLRTAQY
NSKWLNEPNFVAAYDIGLFAYFFLRENAVEHDCGRTVYSRVARVCKNDVGGRFLLEDTWTTFMKARLNCSRPG
EVPFYYNELQSAFHLPEQDLIYGVFTTNVNSIAASAVCAFNLSAISQAFNGPFRYQENPRAAWLPIANPIPNF
QCGTLPETGPNENLTERSLQDAQRLFLMSEAVQPVTPEPCVTQDSVRFSHLVVDLVQAKDTLYHVLYIGTESG
TILKALSTASRSLHGCYLEELHVLPPGRREPLRSLRILHSARALFVGLRDGVLRVPLERCAAYRSQGACLGAR
DPYCGWDGKQQRCSTLEDSSNMSLWTQNITACPVRNVTRDGGFGPWSPWQPCEHLDGDNSGSCLCRARSCDSP
RPRCGGLDCLGPAIHIANCSRNGAWTPWSSWALCSTSCGIGFQVRQRSCSNPAPRHGGRICVGKSREERFCNE
NTPCPVPIFWASWGSWSKCSSNCGGGMQSRRRACENGNSCLGCGVEFKTCNPEGCPEVRRNTPWTPWLPVNVT
QGGARQEQRFRFTCRAPLADPHGLQFGRRRTETRTCPADGSGSCDTDALVEDLLRSGSTSPHTVSGGWAAWGP
WSSCSRDCELGFRVRKRTCTNPEPRNGGLPCVGDAAEYQDCNPQACPVRGAWSCWTSWSPCSASCGGGHYQRT
RSCTSPAPSPGEDICLGLHTEEALCATQACPEGWSPWSEWSKCTDDGAQSRSRHCEELLPGSSACAGNSSQSR
PCPYSEIPVILPASSMEEATGCAGFNLIHLVATGISCFLGSGLLTLAVYLSCQHCQRQSQESTLVHPATPNHL
HYKGGGTPKNEKYTPMEFKTLNKNNLIPDDRANFYPLQQTNVYTTTYYPSPLNKHSFRPEASPGQRCFPNS

| | | |
|---|---|---|
| NOV2h, CG106951-03 | SEQ ID NO: 19 | 1203 bp |
| DNA Sequence | ORF Start: at 7 | ORF Stop: at 1198 |

GGATCCGGCCCATGGTCACCATGGCAACCATGTGAGCACTTGGATGGGGACAACTCAGGCTCTTGCCTGTGTC
GAGCTCGATCCTGTGATTCCCCTCGACCCCGCTGTGGGGGCCTTGACTGCCTGGGGCCAACCATCCACATCGC
CAACTGCTCCAGGAATGGGGCGTGGACCCCGTGGTCATCGTGGGCGCTGTGCAGCACGTCCTGTGGCATCGGC
TTCCAGGTCCGCCAGCGAAGTTGCAGCAACCCTGCTCCCCGCCACGGGGGCCGCATCTGCGTGGGCAAGAGCC
GGGAGGAACGGTTCTGTAATGAGAACACGCCTTGCCCGGTGCCCATCTTCTGGGCTTCCTGGGGCTCCTGGAG
CAAGTGCGGCAGCAACTGTGGAGGGGGCATGCAGTCGCGGCGTCGGGCCTGCGAGAACGGCAACTCCTGCCTG
GGCTGCGGCGTGGAGTTCAAGACGTGCAACCCCGAGGGCTGCCCCGAAGTGCGGCGCAACACCCCCTGGACGC
CGTGGCTGCCCGTGAACGTGACGCAGGGCGGGGCACGGCAGGAGCAGCGGTTCCGCTTCACCTGCCGCGCGCC
CCTTGCAGACCCGCACGGCCTGCAGTTCGGCAGGAGAAGGACCGAGACGAGGACCTGTCCCGCGGACGGCTCC
GGCTCCTGCGACACCGACGCCCTGGTGGAGGTCCTCCTGCGCAGCGGGAGCACCTCCCCGCACACGGTGAGCG
GGGGCTGGGCCGCCTGGGGCCCGTGGTCGTCCTGCTCCCGGGACTCCGAGCTGGGCTTCCGCGTCCGCAAGAG
AACGTGCACTAACCCGGAGCCCCGCAACGGGGGCCTGCCCTGCGTGGGCGATGCTGCCGAGTACCAGGACTGC
AACCCCCAGGCTTGCCCAGTTCGGGGTGCTTGGTCCTGCTGGACCTCATGGTCTCCATGCTCAGCTTCCTGTG
GTGGGGGTCACTATCAACGCACCCGTTCCTGCACCAGCCCCGCACCCTCCCCAGGTGAGGACATCTGTCTCGG
GCTGCACACGGAGGAGGCACTATGTGCCACACAGGCCTGCCCAGAAGGCTGGTCGCCCTGGTCTGAGTGGAGT
AAGTGCACTGACGACGGAGCCCAGAGCCGAAGCCGGCACTGTGAGGAGCTCCTCCCAGGGTCGAGCGCCTGTG
CTGGAAACAGCAGCCAGAGCCGCCCCTGCGTCGAC

| | | | |
|---|---|---|---|
| NOV2h, CG106951-03 | | | |
| Protein Sequence | SEQ ID NO: 20 | 397 aa | MW at 42882.1kD |

GPWSPWQPCEHLDGDNSGSCLCRARSCDSPRPRCGGLDCLGPTIHIANCSRNGAWTPWSSWALCSTSCGIGFQ
VRQRSCSNPAPRHGGRICVGKSREERFCNENTPCPVPIFWASWGSWSKCGSNCGGGMQSRRRACENGNSCLGC
GVEFKTCNPEGCPEVRRNTPWTPWLPVNVTQGGARQEQRFRFTCRAPLADPHGLQFGRRRTETRTCPADGSGS
CDTDALVEVLLRSGSTSPHTVSGGWAAWGPWSSCSRDCELGFRVRKRTCTNPEPRNGGLPCVGDAAEYQDCNP
QACPVRGAWSCWTSWSPCSASCGGGHYQRTRSCTSPAPSPGEDICLGLHTEEALCATQACPEGWSPWSEWSKC
TDDGAQSRSRHCEELLPGSSACAGNSSQSRPC

| | | |
|---|---|---|
| | SEQ ID NO: 21 | 6408 bp |
| NOV2i, SNP13382456 of | ORF Start: ATG at 1400 | ORF Stop: TGA at 5456 |
| CG106951-01, DNA Sequence | SNP Pos: 5770 | SNP Change: C to T |

CCTGGGACTCTGGGAGAATGGTCCAGAGCTCATTGTCCTTGTTGATAAAATGATAGATTTGGACTCAATATCC
CATGCTGCCTCTTCCAACTTGATTTTTACCCCAGACTGGGCTACCAGACTGGTATGCCCACACATGCCCGTTT
CCTTTCTTTTCTTCTCTGCATCTCTGCCTTTGTGTCCAGAGCGTGTTTTCCCTTTGCAAGTTTCTCTCCATTC
TGCACATTATGAGTTTCAGCATTTCTGTTGCCCTAGAAAGTCTATCTTTGAGATCTTGCACTGTTTCTCTTTT
TACAGTGTCTCATAAACTCCCTTCTTGGATTCAGAACCACCCTTTCTTTCCCATTATCCTGTCAAACTGCTTC
TTGCCATGGTCCAGGGGTAGGAGGATGGCAGGCAGGAGGTGCTTCTCTGGGGCTCTTAGTGTCTCAATTCTTC
TGCTTTATCTGGGTTTTCCTTTACCCAGAATTTTATTATGTAAAATGCTTCACTCAGACTTTGTTCTAATTAT
CCAATTTTTGGCATACTCTAGAAAGTCTTTTGATATTTTCCTTCCTCCAACTTATCTATTTTTATTTCATAGT
TCTCTTTGGTTATCTCTTAGAATCACACTTTCCTGGTTTTAATTTTTCAAATCCTTTGTCTTTCTCACTCGTT
CTTAGGTCACCTTTTTTTACATTTTCAAATATATTTTTTGTTCAGCAGAGGGCTCCCTTCCCATCCCTCTTGC
AGCCCGGGCAGCTAGGATTTGAAGCTTGCCCCTTGAATCTTTCTCTCCCGCCTTCTAGCCATCAGAAACACTA
GATCACTTAAACTTGTAAACAATTCGGCCTCGCTCCTTGTGATTGCGCTAAACCTTCCGTCCTCAGCTGAGAA
CGCTCCACCACCTCCCCGGATCGCTCATCTCTTGGCTGCCCTCCCACTGTTCCTGATGTTATTTTACTCCCCG
TATCCCCTACTCGTTCTTCACAATTCTGTAGGGTGCGTATTACTAACCCCAGTTTACAGCTGAGGAAACTGAG
GCTTGGAGAGGTTCGCTCGGTATCGTACAGTTTGCAAGGTTAACCCTAATCCGGCCAGTTCTGGCTTTCCAGC
CCAGCCCAGCAGCCTAGCCTCCCTCTCTGCCGCTGCAGGTTATAACGGCTCTCCCCCGTTTTACACGAGGTCC
CTTCCCCTTCAAATCCACAGGCAGGAAGATCGTTCCGAACTGACGGGGCTGGGGAATGTGGGAGTCCGGAGTG
GGGTTTGGGGGAGCTTCCTCAGGCCCTGAGTGTTGGGGTGGGCAGGCCGCGCCGATGGCCCTCGGGGATGTCA
CATTCGAGATGGGGTGACCGAGAACGGCAAGGCGGGATGTGGCAAACGGCGGCAAGTGCTCGGAGTCCTAGGT
CTTGCCGCCGGAATGCCGGCCGGGGAAGGGGCTTCGGCCCACCGGGCTGGTCACCACACTCGGCAGGCCCGGG
GCGGGAGTCGGCCGAGCAGCCGCGGGATGCAGGGCGCCCCCTCGCGCTCCTCCGCGCGCCTCGAGGCTGGCGG
GTGCAGCGCCCGCCGCGGCAGGTCTGCTCCAGCCCCCTCCTCTTTTTCGCTCCCGCTCCCCTCCTTCTCTCCC
TTTGCTTGCAACTCCTCCCCCACCGCCCCCTCCCTCCTTCTGCTCCCGCGGTCTCCTCCTCCCTGCTCTCTCC
GAGCGCCGGGTCGGGAGCTAGTTGGAGCGCGGGGGTTGGTGCCAGAGCCCAGCTCCGCCGAGCCGGGCGGGTC
GGCAGCGCATCCAGCGGCTGCTGGGAGCCCGAGCGCAGCGGGCGCGGGCCCGGGTGGGGACTGCACCGGAGCG
CTGAGAGCTGGAGGCCGTTCCTGCGCGGCCGCCCCATTCCCAGACCGGCCGCCAGCCCATCTGGTTAGCTCCC
GCCGCTCCGCGCCGCCCGGGAGTCGGGAGCCGCGGGGAACCGGGCACCTGCACCCGCCTCTGGGAGTGAGTGG
TTCCAGCTGGTGCCTGGCCTGTGTCTCTTGGATGCCCTGTGGCTTCAGTCCGTCTCCTGTTGCCCACCACCTC
GTCCCTGGGCCGCCTGATACCCCAGCCCAACAGCTAAGGTGTGGATGGACAGTAGGGGGCTGGCTTCTCTCAC
TGGTCAGGGGTCTTCTCCCCTGTCTGCCTCCCGGAGCTAGGACTGCAGAGGGGCCTATCATGGTGCTTGCAGG
CCCCCTGGCTGTCTCGCTGTTGCTGCCCAGCCTCACACTGCTGGTGTCCCACCTCTCCAGCTCCCAGGATGTC
TCCAGTGAGCCCAGCAGTGAGCAGCAGCTGTGCGCCCTTAGCAAGCACCCCACCGTGGCCTTTGAAGACCTGC
AGCCGTGGGTCTCTAACTTCACCTACCCTGGAGCCCGGGATTTCTCCCAGCTGGCTTTGGACCCCTCCGGGAA
CCAGCTCATCGTGGGAGCCAGGAACTACCTCTTCAGACTCAGCCTTGCCAATGTCTCTCTTCTTCAGGCCACA
GAGTGGGCCTCCAGTGAGGACACGCGCCGCTCCTGCCAAAGCAAAGGGAAGACTGAGGAGGAGTGTCAGAACT
ACGTGCGAGTCCTGATCGTCGCCGGCCGGAAGGTGTTCATGTGTGGAACCAATGCCTTTTCCCCCATGTGCAC
CAGCAGACAGGTGGGGAACCTCAGCCGGACTACTGAGAAGATCAATGGTGTGGCCCGCTGCCCCTATGACCCA
CGCCACAACTCCACAGCTGTCATCTCCTCCCAGGGGGAGCTCTATGCAGCCACGGTCATCGACTTCTCAGGTC
GGGACCCTGCCATCTACCGCAGCCTGGGCAGTGGGCCACCGCTTCGCACTGCCCAATATAACTCCAAGTGGCT
TAATGAGCCAAACTTCGTGGCAGCCTATGATATTGGGCTGTTTGCATACTTCTTCCTGCGGGAGAACGCAGTG
GAGCACGACTGTGGACGCACCGTGTACTCTCGCGTGGCCCGCGTGTGCAAGAATGACGTGGGGGGCCGATTCC
TGCTGGAGGACACATGGACCACATTCATGAAGGCCCGGCTCAACTGCTCCCGCCCGGGCGAGGTCCCCTTCTA
CTATAACGAGCTGCAGAGTGCCTTCCACTTGCCAGAGCAGGACCTCATCTATGGAGTTTTCACAACCAACGTA
AACAGCATCGCGGCTTCTGCTGTCTGCGCCTTCAACCTCAGTGCTATCTCCCAGGCTTTCAATGGCCCATTTC
GCTACCAGGAGAACCCCAGGGCTGCCTGGCTCCCCATAGCCAACCCCATCCCCAATTTCCAGTGTGGCACCCT
GCCTGAGACCGGTCCCAACGAGAACCTGACGGAGCGCAGCCTGCAGGACGCGCAGCGCCTCTTCCTGATGAGC
GAGGCCGTGCAGCCGGTGACACCCGAGCCCTGTGTCACCCAGGACAGCGTGCGCTTCTCACACCTCGTGGTGG
ACCTGGTGCAGGCTAAAGACACGCTCTACCATGTACTCTACATTGGCACCGAGTCGGGCACCATCCTGAAGGC
GCTGTCCACGGCGAGCCGCAGCCTCCACGGCTGCTACCTGGAGGAGCTGCACGTGCTGCCCCCCGGGCGCCGC
GAGCCCCTGCGCAGCCTGCGCATCCTGCACAGCGCCCGCGCGCTCTTCGTGGGGCTGAGAGACGGCGTCCTGC
GGGTCCCACTGGAGAGGTGCGCCGCCTACCGCAGCCAGGGGGCATGCCTGGGGGCCCGGGACCCGTACTGTGG
CTGGGACGGGAAGCAGCAACGTTCCAGCACACTCGAGGACAGCTCCAACATGAGCCTCTGGACCCAGAACATC
ACCGCCTGTCCTGTGCGGAATGTGACACGGGATGGGGGCTTCGGCCCATGGTCACCATGGCAACCATGTGAGC
ACTTGGATGGGGACAACTCAGGCTCTTGCCTGTGTCGAGCTCGATCCTGTGATTCCCCTCGACCCCGCTGTGG
GGGCCTTGACTGCCTGGGGCCAGCCATCCACATCGCCAACTGCTCCAGGAATGGGGCGTGGACCCCGTCGTCA
TCGTGGGCGCTGTGCAGCACGTCCTGTGGCATCGGCTTCCAGGTCCGCCAGCGAAGTTGCAGCAACCCTGCTC
CCCGCCACGGGGGCCGCATCTGCGTGGGCAAGAGCCGGGAGGAACGGTTCTGTAATGAGAACACGCCTTGCCC
GGTGCCCATCTTCTGGGCTTCCTGGGGCTCCTGGAGCAAGTGCAGCAGCAACTGTGGAGGGGGCATGCAGTCG
CGGCGTCGGGCCTGCGAGAACGGCAACTCCTGCCTGGGCTGCGGCGTGGAGTTCAAGACGTGCAACCCCGAGG
GCTGCCCCGAAGTGCGGCGCAACACCCCCTGGACGCCGTGGCTGCCCGTGAACGTGACGCAGGGCGGGGCACG
GCAGGAGCAGCGGTTCCGCTTCACCTGCCGCGCGCCCCTTGCAGACCCGCACGGCCTGCAGTTCGGCAGGAGA
AGGACCGAGACGAGGACCTGTCCCGCGGACGGCTCCGGCTCCTGCGACACCGACGCCCTGGTGGAGGACCTCC
TGCGCAGCGGGAGCACCTCCCCGCACACGGTGAGCGGGGGCTGGGCCGCCTGGGGCCCGTGGTCGTCCTGCTC
CCGGGACTGCGAGCTGGGCTTCCGCGTCCGCAAGAGAACGTGCACTAACCCGGAGCCCCGCAACGGGGGCCTG
CCCTGCGTGGGCGATGCTGCCGAGTACCAGGACTGCAACCCCCAGGCTTGCCCAGTTCGGGCTGCTTGGTCCT
GCTGGACCTCATGGTCTCCATGCTCAGCTTCCTGTGGTGGGGGTCACTATCAACGCACCCGTTCCTGCACCAG
CCCCGCACCCTCCCCAGGTGAGGACATCTGTCTCGGGCTGCACACGGAGGAGGCACTATGTGCCACACAGGCC
TGCCCAGAAGGCTGGTCGCCCTGGTCTGAGTGGAGTAAGTGCACTGACGACGGAGCCCAGAGCCGAAGCCGGC
ACTGTGAGGAGCTCCTCCCAGGGTCCAGCGCCTGTGCTGGAAACAGCAGCCAGAGCCGCCCCTGCCCCTACAG
CGAGATTCCCGTCATCCTGCCAGCCTCCAGCATGGAGGAGGCCACCGGCTGTGCAGGGTTCAATCTCATCCAC
TTGGTGGCCACGGGCATCTCCTGCTTCTTGGGCTCTGGGCTCCTGACCCTAGCAGTGTACCTGTCTTGCCAGC
ACTGCCAGCGTCAGTCCCAGGAGTCCACACTGGTCCATCCTGCCACCCCCAACCATTTGCACTACAAGGGCGG
AGGCACCCCGAAGAATGAAAAGTACACACCCATGGAATTCAAGACCCTGAACAAGAATAACTTGATCCCTGAT
GACAGAGCCAACTTCTACCCATTGCAGCAGACCAATGTGTACACGACTACTTACTACCCAAGCCCCCTGAACA
AACACAGCTTCCGGCCCGAGGCCTCACCTGGACAACGGTGCTTCCCCAACAGCTGATACCGCCGTCCTGGGGA
CTTGGGCTTCTTGCCTTCATAAGGCACAGAGCAGATGGAGATGGGACAGTGGAGCCAGTTTGGTTTTCTCCCT
CTGCACTAGGCCAAGAACTTGCTGCCTTGCCTGTGGGGGGTCCCATCCGGCTTCAGAGAGCTCTGGCTGGCAT
TGACCATGGGGGAAAGGGCTGGTTTCAGGCTGACATATGGCCGCAGGTCCAGTTCAGCCCAGGTCTCTCATGG
TTATCTTCCAACCCACTGTCACGCTGACACTATGCTGCCATGCCTGGGCTGTGGACCTACTGGGCATTTGAGG
AATTGGAGAATGGAGATGGCAAGAGGGCAGGCTTTTAAGTTTGGGTTGGAGACAACTTCCTGTGGCCCCCACA
AGCTGAGTCTGGCCTTCTCCAGCTGGCCCCAAAAAAGGCCTTTGCTACATCCTGATTATCTCTGAAAGTAATC
AATCAAGTGGCTCCAGTAGCTCTGGATTTTCTGCCAGGGCTGGGCCATTGTGGTGCTGCCCCAGTATGACATG
GGACCAAGGCCAGCGCAGGTTATCCACCTCTGCCTGGAAGTCTATACTCTACCCAGGGCATCCCTCTGGTCAG
AGGCAGTGAGTACTGGGAACTGGAGGCTGACCTGTGCTTAGAAGTCCTTTAATCTGGGCTGGTACAGGCCTCA
GCCTTGCCCTCAATGCACGAAAGGTGGCCCAGGAGAGAGGATCAATGCCACAGGAGGCAGAAGTCTGGCCTCT
GTGCCTCTATGGAGACTATCTTCCAGTTGCTGCTCAACAGAGTTGTTGGCTGAGACCTGCTTGGGAGTCTCTG
CTGGCCCTTCATCTGTTCAGGAACACACACACACACACACTCACACACGCACACACAATCACAATTTGCTACA
GCAACAAAAAAGACATTGGGCTGTGGCATTATTAATTAAAGATGATATCCAGTCTCC

| | | | |
|---|---|---|---|
| NOV2i, SNP13382456 of | | | MW at 145674.1kD |
| CG106951-01, Protein Sequence | SEQ ID NO: 22 | 1352 aa | SNP Change: no change |

MPAGEGASAHRAGHHTRQARGGSRPSSRGMQGAPSRSSARLEAGGCSARRGRSAPAPSSFSLPLPSFSPFACN
SSPTAPSLLLLPRSPPPCSLRAPGRELVGARGLVPEPSSAEPGGSAAHPAAAGSPSAAGAGPGGDCTGALRAG
GRSCAAAPFPDRPPAHLVSSRRSAPPGSREPRGTGHLHPPLGVSGSSWCLACVSWMPCGFSPSPVAHHLVPGP
PDTPAQQLRCGWTVGGWLLSLVRGLLPCLPPGARTAEGPIMVLAGPLAVSLLLPSLTLLVSHLSSSQDVSSEP
SSEQQLCALSKHPTVAFEDLQPWVSNFTYPGARDFSQLALDPSGNQLIVGARNYLFRLSLANVSLLQATEWAS
SEDTRRSCQSKGKTEEECQNYVRVLIVAGRKVFMCGTNAFSPMCTSRQVGNLSRTTEKINGVARCPYDPRHNS
TAVISSQGELYAATVIDFSGRDPAIYRSLGSGPPLRTAQYNSKWLNEPNFVAAYDIGLFAYFFLRENAVEHDC
GRTVYSRVARVCKNDVGGRFLLEDTWTTFMKARLNCSRPGEVPFYYNELQSAFHLPEQDLIYGVFTTNVNSIA
ASAVCAFNLSAISQAFNGPFRYQENPRAAWLPIANPIPNFQCGTLPETGPNENLTERSLQDAQRLFLMSEAVQ
PVTPEPCVTQDSVRFSHLVVDLVQAKDTLYHVLYIGTESGTILKALSTASRSLHGCYLEELHVLPPGRREPLR
SLRILHSARALFVGLRDGVLRVPLERCAAYRSQGACLGARDPYCCWDGKQQRCSTLEDSSNMSLWTQNITACP
VRNVTRDGGFGPWSPWQPCEHLDGDNSGSCLCRARSCDSPRPRCGGLDCLGPAIHIANCSRNGAWTPWSSWAL
CSTSCGIGFQVRQRSCSNPAPRHGGRICVGKSREERFCNENTPCPVPIFWASWGSWSKCSSNCGGCMQSRRRA
CENGNSCLGCGVEFKTCNPEGCPEVRRNTPWTPWLPVNVTQGGARQEQRFRFTCRAPLADPHGLQFGRRRTET
RTCPADGSGSCDTDALVEDLLRSGSTSPHTVSGGWAAWGPWSSCSRDCELGFRVRKRTCTNPEPRNGGLPCVG
DAAEYQDCNPQACPVRGAWSCWTSWSPCSASCGGGHYQRTRSCTSPAPSPGEDICLGLHTEEALCATQACPEG
WSPWSEWSKCTDDGAQSRSRHCEELLPGSSACAGNSSQSRPCPYSEIPVILPASSMEEATGCAGFNLIHLVAT
GISCFLGSGLLTLAVYLSCQHCQRQSQESTLVHPATPNHLHYKGGGTPKNEKYTPMEFKTLNKNNLIPDDRAN
FYPLQQTNVYTTTYYPSPLNKHSFRPEASPGQRCFPNS

A ClustalW comparison of the above protein sequences yields the following sequence alignment shown in Table 2B.

TABLE 2B

| Comparison of the NOV2 protein sequences. | |
|---|---|
| NOV2a | MPAGEGASAHRAGHHTRQARGGSRPSSRGMQGAPSRSSARLEAGGCSARRGRSAPAPSSF |
| NOV2b | ------------------------------------------------------------ |
| NOV2c | ------------------------------------------------------------ |
| NOV2d | ------------------------------------------------------------ |
| NOV2e | ------------------------------------------------------------ |
| NOV2f | ------------------------------------------------------------ |
| NOV2g | ------------------------------------------------------------ |
| NOV2h | ------------------------------------------------------------ |
| NOV2a | SLPLPSFSPFACNSSPTAPSLLLLPRSPPPCSLRAPGRELVGARGLVPEPSSAEPGGSAA |
| NOV2b | ------------------------------------------------------------ |
| NOV2c | ------------------------------------------------------------ |
| NOV2d | ------------------------------------------------------------ |
| NOV2e | ------------------------------------------------------------ |
| NOV2f | ------------------------------------------------------------ |
| NOV2g | ------------------------------------------------------------ |
| NOV2h | ------------------------------------------------------------ |
| NOV2a | HPAAAGSPSAAGAGPGGDCTGALRAGGRSCAAAPFPDRPPAHLVSSRRSAPPGSREPRGT |
| NOV2b | ------------------------------------------------------------ |
| NOV2c | ------------------------------------------------------------ |
| NOV2d | ------------------------------------------------------------ |
| NOV2e | ------------------------------------------------------------ |
| NOV2f | ------------------------------------------------------------ |
| NOV2g | ------------------------------------------------------------ |
| NOV2h | ------------------------------------------------------------ |
| NOV2a | GHLHPPLGVSGSSWCLACVSWMPCGFSPSPVAHHLVPGPPDTPAQQLRCGWTVGGWLLSL |
| NOV2b | ---------------------MPCGFSPSPVAHHLVPGPPDTPAQQLRCGWTVGGWLLSL |
| NOV2c | ------------------------------------------------------------ |
| NOV2d | ------------------------------------------------------------ |
| NOV2e | ------------------------------------------------------------ |
| NOV2f | ------------------------------------------------------------ |
| NOV2g | ------------------------------------------------------------ |
| NOV2h | ------------------------------------------------------------ |
| NOV2a | VRGLLPCLPPGARTAEGPIMVLAGPLAVSLLLPSLTLLVSHLSSSQDVSSEPSSEQQLCA |
| NOV2b | VRGLLPCLPPGARTAEGPIMVLAGPLAVSLLLPSLTLLVSHLSSSQDVSSEPSSEQQLCA |
| NOV2c | ------------------------------------------------------------ |
| NOV2d | ------------------------------------------------------------ |
| NOV2e | ------------------------------------------------------------ |
| NOV2f | ------------------------------------------------------------ |
| NOV2g | -------------------MVLAGPLAVSLLLPSLTLLVSHLSSSQDVSSEPSSEQQLCA |
| NOV2h | ------------------------------------------------------------ |
| NOV2a | LSKHPTVAFEDLQPWVSNFTYPGARDFSQLALDPSGNQLIVGARNYLFRLSLANVSLLQA |
| NOV2b | LSKHPTVAFEDLQPWVSNFTYPGARDFSQLALDPSGNQLIVGARNYLFRLSLANVSLLQA |
| NOV2c | ------------------------------------------------------------ |
| NOV2d | ------------------------------------------------------------ |
| NOV2e | ------------------------------------------------------------ |
| NOV2f | ------------------------------------------------------------ |
| NOV2g | LSKHPTVAFEDLQPWVSNFTYPGARDFSQLALDPSGNQLIVGARNYLFRLSLANVSLLQA |
| NOV2h | ------------------------------------------------------------ |
| NOV2a | TEWASSEDTRRSCQSKGKTEEECQNYVRVLIVAGRKVFMCGTNAFSPMCTSRQVGNLSRT |
| NOV2b | TEWASSEDTRRSCQSKGKTEEECQNYVRVLIVAGRKVFMCGTNAFSPMCTSRQVGNLSRT |
| NOV2c | ------------------------------------------------------------ |
| NOV2d | ------------------------------------------------------------ |
| NOV2e | ------------------------------------------------------------ |
| NOV2f | ------------------------------------------------------------ |
| NOV2g | TEWASSEDTRRSCQSKGKTEEECQNYVRVLIVAGRKVFMCGTNAFSPMCTSRQVGNLSRT |
| NOV2h | ------------------------------------------------------------ |
| NOV2a | TEKINGVARCPYDPRHNSTAVISSQGELYAATVIDFSGRDPAIYRSLGSGPPLRTAQYNS |
| NOV2b | TEKINGVARCPYDPRHNSTAVISSQGELYAATVIDFSGRDPAIYRSLGSGPPLRTAQYNS |
| NOV2c | ------------------------------------------------------------ |
| NOV2d | ------------------------------------------------------------ |
| NOV2e | ------------------------------------------------------------ |
| NOV2f | ------------------------------------------------------------ |
| NOV2g | TEKINGVARCPYDPRHNSTAVISSQGELYAATVIDFSGRDPAIYRSLCSGPPLRTAQYNS |
| NOV2h | ------------------------------------------------------------ |
| NOV2a | KWLNEPNFVAAYDIGLFAYFFLRENAVEHDCGRTVYSRVARVCKNDVGGRFLLEDTWTTF |
| NOV2b | KWLNEPNFVAAYDIGLFAYFFLRENAVEHDCGRTVYSRVARVCKNDVGGRFLLEDTWTTF |
| NOV2c | ------------------------------------------------------------ |
| NOV2d | ------------------------------------------------------------ |
| NOV2e | ------------------------------------------------------------ |
| NOV2f | ------------------------------------------------------------ |
| NOV2g | KWLNEPNFVAAYDIGLFAYFFLRENAVEHDCGRTVYSRVARVCKNDVGGRFLLEDTWTTF |
| NOV2h | ------------------------------------------------------------ |
| NOV2a | MKARLNCSRPGEVPFYYNELQSAFHLPEQDLIYGVFTTNVNSIAASAVCAFNLSAISQAF |
| NOV2b | MKARLNCSRPGEVPFYYNELQSAFHLPEQDLIYGVFTTNVNSIAASAVCAFNLSAISQAF |
| NOV2c | ------------------------------------------------------------ |
| NOV2d | ------------------------------------------------------------ |
| NOV2e | ------------------------------------------------------------ |
| NOV2f | ------------------------------------------------------------ |
| NOV2g | MKARLNCSRPGEVPFYYNELQSAFHLPEQDLIYGVFTTNVNSIAASAVCAFNLSAISQAF |
| NOV2h | ------------------------------------------------------------ |
| NOV2a | NGPFRYQENPRAAWLPIANPIPNFQCGTLPETGPNENLTERSLQDAQRLFLMSEAVQPVT |
| NOV2b | NGPFRYQENPRAAWLPIANPIPNFQCGTLPETGPNENLTERSLQDAQRLFLMSEAVQPVT |
| NOV2c | ------------------------------------------------------------ |
| NOV2d | ------------------------------------------------------------ |
| NOV2e | ------------------------------------------------------------ |
| NOV2f | ------------------------------------------------------------ |
| NOV2g | NGPFRYQENPRAAWLPIANPIPNFQCGTLPETGPNENLTERSLQDAQRLFLMSEAVQPVT |
| NOV2h | ------------------------------------------------------------ |
| NOV2a | PEPCVTQDSVRFSHLVVDLVQAKDTLYHVLYIGTESGTILKALSTASRSLHGCYLEELHV |
| NOV2b | PEPCVTQDSVRFSHLVVDLVQAKDTLYHVLYIGTESGTILKALSTASRSLHGCYLEELHV |
| NOV2c | ------------------------------------------------------------ |
| NOV2d | ------------------------------------------------------------ |
| NOV2e | ------------------------------------------------------------ |
| NOV2f | ------------------------------------------------------------ |
| NOV2g | PEPCVTQDSVRFSHLVVDLVQAKDTLYHVLYIGTESGTILKALSTASRSLHGCYLEELHV |
| NOV2h | ------------------------------------------------------------ |
| NOV2a | LPPGRREPLRSLRILHSARALFVGLRDGVLRVPLERCAAYRSQGACLGARDPYCGWDGKQ |
| NOV2b | LPPGRREPLRSLRILHSARALFVGLRDGVLRVPLERCAAYRSQGACLGARDPYCGWDGKQ |
| NOV2c | ------------------------------------------------------------ |
| NOV2d | ------------------------------------------------------------ |
| NOV2e | ------------------------------------------------------------ |
| NOV2f | ------------------------------------------------------------ |
| NOV2g | LPPGRREPLRSLRILHSARALFVGLRDGVLRVPLERCAAYRSQGACLGARDPYCGWDGKQ |
| NOV2h | ------------------------------------------------------------ |
| NOV2a | QRCSTLEDSSNMSLWTQNITACPVRNVTRDGGFGPWSPWQPCEHLDGDNSGSCLCRARSC |
| NOV2b | QRCSTLEDSSNMSLWTQNITACPVRNVTRDGGFGPWSPWQPCEHLDGDNSGSCLCRARSC |
| NOV2c | -------------------------------GSGPWSPWQPCEHLDGDNSGSCLCRARSC |
| NOV2d | -------------------------------GSGPWSPWQPCEHLDGDNSGSCLCRARSC |
| NOV2e | -------------------------------GSGPWSPWQPCEHLDGDNSGSCLCRARSC |
| NOV2f | -------------------------------GSGPWSPWQPCEHLDGDNSGSCLCRARSC |
| NOV2g | QRCSTLEDSSNMSLWTQNITACPVRNVTRDGGFGPWSPWQPCEHLDGDNSGSCLCRARSC |
| NOV2h | ---------------------------------GPWSPWQPCEHLDGDNSGSCLCRARSC |
| NOV2a | DSPRPRCGGLDCLGPAIHIANCSRNGAWTPWSSWALCSTSCGIGFQVRQRSCSNPAPRHG |
| NOV2b | DSPRPRCGGLDCLGPAIHIANCSRNGAWTPWSSWALCSTSCGIGFQVRQRSCSNPAPHG |
| NOV2c | DSPRPRCGGLDCLGPAIHIANCSRNGAWTPWSSWALCSTSCGIGFQVRQRSCSNPAPRHG |
| NOV2d | DSPRPRCGGLDCLGPAIHIANCSRNGAWTPWSSWALCSTSCGIGFQVRQRSCSNPAPRHG |
| NOV2e | DSPRPRCGGLDCLGPAIHIANCSRNGAWTPWSSWALCSTSCGIGFQVRQRSCSNPAPRHG |
| NOV2f | DSPRPRCGGLDCLGPTIHIANCSRNGAWTPWSSWALCSTSCGIGFQVRQRSCSNPAPRHG |
| NOV2g | DSPRPRCGGLDCLGPAIHIANCSRNGAWTPWSSWALCSTSCGIGFQVRQRSCSNPAPRHG |
| NOV2h | DSPRPRCGGLDCLGPTIHIANCSRNGAWTPWSSWALCSTSCGIGFQVRQRSCSNPAPRHG |
| NOV2a | GRICVGKSREERFCNENTPCPVPIFWASWGSWSKCSSNCGGGMQSRRRACENGNSCLGCG |
| NOV2b | GRICVGKSREERFCNENTPCPVPIFWASWGSWSKCSSNCGGGMQSRRRACENGNSCLGCG |
| NOV2c | GRICVGKSREERFCNENTPCPVPIFWASWGSWSKCSSNCGGGMRSRRRACENGNSCLGCG |
| NOV2d | GRICVGKSREERFCNENTPCPVPIFWASWGSWSKCSSNCGGGMQSRRRACENGNSCLGCG |
| NOV2e | GRICVGKSREERFCNENTPCPVPIFWASWGSWSKCSSNCGGGMQSRRRACENGNSCLGCG |
| NOV2f | GRICVGKSREERFCNENTPCPVPIFWASWGSWSKCGSNCGGGMQSRRRACENGNSCLGCG |
| NOV2g | GRICVGKSREERFCNENTPCPVPIFWASWGSWSKCSSNCGGGMQSRRRACENGNSCLGCG |
| NOV2h | GRICVGKSREERFCNENTPCPVPIFWASWGSWSKCGSNCGGGMQSRRRACENGNSCLGCG |
| NOV2a | VEFKTCNPEGCPEVRRNTPWTPWLPVNVTQGGARQEQRFRFTCRAPLADPHGLQFGRRRT |
| NOV2b | VEFKTCNPEGCPEVRRNTPWTPWLPVNVTQGGARQEQRFRFTCRAPLADPHGLQFGRRRT |
| NOV2c | VEFKTCNPEGCPEVRRNTPWTPWLPVNVTQGGARQEQRFRFTCRAPLADPHGLQFGRRRT |
| NOV2d | VEFKTCNPEGCPEVRRNTPWTPWLPVNVTQGGARQEQRFRFTCRAPLADPHGLQFGRRRT |
| NOV2e | VEFKTCNPEGCPEVRRNTPWTPWLPVNVTQGGARQEQRFRFTCRAPLADPHGLQFGRRRT |
| NOV2f | VEFKTCNPEGCPEVRRNTPWTPWLPVNVTQGGARQEQRFRFTCRAPLADPHGLQFGRRRT |
| NOV2g | VEFKTCNPEGCPEVRRNTPWTPWLPVNVTQGGARQEQRFRFTCRAPLADPHGLQFGRRRT |
| NOV2h | VEFKTCNPEGCPEVRRNTPWTPWLPVNVTQGGARQEQRFRFTCRAPLADPHGLQFGRRRT |
| NOV2a | ETRTCPADGSGSCDTDALVEDLLRSGSTSPHTVSGGWAAWGPWSSCSRDCELGFRVRKRT |
| NOV2b | ETRTCPADGSGSCDTDALVEDLLRSGSTSPHTVSGGWAAWGPWSSCSRDCELGFRVRKRT |
| NOV2c | ETRTCPADGSGSCDTDALVEDLLRSGSTSPHTVSGGWAAWGPWSSCSRDCELGFRVRKRT |
| NOV2d | ETRTCPADGSGSCDTDALVEDLLRSGSTSPHTVSGGWAAWGPWSSCSRDCELGFRVRKRT |
| NOV2e | ETRTCPADGSGSCDTDALVEDLLRSGSTSPHTVSGGWAAWGPWSSCSRDCELGFRVRKRT |
| NOV2f | ETRTCPADGSGSCDTDALVEVLLRSGSTSPHTVSGGWAAWGPWSSCSRDCELGFRVRKRT |
| NOV2g | ETRTCPADGSGSCDTDALVEDLLRSGSTSPHTVSGGWAAWGPWSSCSRDCELGFRVRKRT |
| NOV2h | ETRTCPADGSGSCDTDALVEVLLRSGSTSPHTVSGGWAAWGPWSSCSRDCELGFRVRKRT |
| NOV2a | CTNPEPRNGGLPCVGDAAEYQDCNPQACPVRGAWSCWTSWSPCSASCGGGHYQRTRSCTS |
| NOV2b | CTNPEPRNGGLPCVGDAAEYQDCNPQACPVRGAWSCWTSWSPCSASCGGGHYQRTRSCTS |
| NOV2c | CTNPEPRNGGLPCVGDAAEYQDCNPQACPVRGAWSCWTSWSPCSASCGGGHYQRTRSCTS |
| NOV2d | CTNPESRNGGLPCVGDAAEYQDCNPQACPVRGAWSCWTSWSPCSASCGGGHYQRTRSCTS |
| NOV2e | CTNPEPRNGGLPCVGDAAEYQDCNPQACPVRGAWSCWTSWSPCSASCGGGHYQRTRSCTS |
| NOV2f | CTNPEPRNGGLPCVGDAAEYQDCNPQACPVRGAWSCWTSWSPCSASCGGGHYQRTRSCTS |
| NOV2g | CTNPEPRNGGLPCVGDAAEYQDCNPQACPVRGAWSCWTSWSPCSASCGGGHYQRTRSCTS |
| NOV2h | CTNPEPRNGGLPCVGDAAEYQDCNPQACPVRGAWSCWTSWSPCSASCGGGHYQRTRSCTS |
| NOV2a | PAPSPGEDICLGLHTEEALCATQACPEGWSPWSEWSKCTDDGAQSRSRHCEELLPGSSAC |
| NOV2b | PAPSP---------------------EGWSPWSEWSKCTDDGAQSRSRHCEELLPGSSAC |
| NOV2c | PAPSPGEDICLGLHTEEALCATQACPEGWSPWSEWSKCTDDGAQSRSRHCEELLPGSSAC |
| NOV2d | PAPSPGEDICLGLHTEEALCATQACPEGWSPWSEWSKCTDDGAQSRSRHCEELLPGSSAC |
| NOV2e | PAPSPGEDICLGLHTEEALCATQACPEGWSPWSEWSKCTDDGAQSRSRHCEELLPGSSAC |
| NOV2f | PAPSPGEDICLGLHTEEALCATQACPEGWSPWSEWSKCTDDGAQSRSRHCEELLPGSSAC |
| NOV2g | PAPSPGEDICLGLHTEEALCATQACPEGWSPWSEWSKCTDDGAQSRSRHCEELLPGSSAC |
| NOV2h | PAPSPGEDICLGLHTEEALCATQACPEGWSPWSEWSKCTDDGAQSRSRHCEELLPGSSAC |
| NOV2a | AGNSSQSRPCPYSEIPVILPASSMEEATGCAGFNLIHLVATGISCFLGSGLLTLAVYLSC |
| NOV2b | AGNSSQSRPCPYSEIPVILPASSMEEATGCAGFNLIHLVATGISCFLGSGLLTLAVYLSC |
| NOV2c | AGNSSQSRPCVD------------------------------------------------ |
| NOV2d | AGNSSQSRPCVD------------------------------------------------ |
| NOV2e | AGNSSQSRPCVD------------------------------------------------ |
| NOV2f | AGNSSQSRPCVD------------------------------------------------ |
| NOV2g | AGNSSQSRPCPYSEIPVILPASSMEEATGCAGFNLIHLVATGISCFLGSGLLTLAVYLSC |
| NOV2h | AGNSSQSRPC-------------------------------------------------- |
| NOV2a | QHCQRQSQESTLVHPATPNHLHYKGGGTPKNEKYTPMEFKTLNKNNLIPDDRANFYPLQQ |
| NOV2b | QHCQRQSQESTLVHPATPNHLHYKGGGTPKNEKYTPMEFKTLNKNNLIPDDRANFYPLQQ |
| NOV2c | ------------------------------------------------------------ |
| NOV2d | ------------------------------------------------------------ |
| NOV2e | ------------------------------------------------------------ |
| NOV2f | ------------------------------------------------------------ |
| NOV2g | QHCQRQSQESTLVHPATPNHLHYKGGGTPKNEKYTPMEFKTLNKNNLIPDDRANFYPLQQ |
| NOV2h | ------------------------------------------------------------ |
| NOV2a | TNVYTTTYYPSPLNKHSFRPEASPGQRCFPNS |
| NOV2b | TNVYTTTYYPSPLNKHSFRPEASPGQRCFPNS |
| NOV2c | -------------------------------- |
| NOV2d | -------------------------------- |
| NOV2e | -------------------------------- |
| NOV2f | -------------------------------- |
| NOV2g | TNVYTTTYYPSPLNKHSFRPEASPGQRCFPNS |
| NOV2h | -------------------------------- |
| NOV2a | (SEQ ID NO: 6) |
| NOV2b | (SEQ ID NO: 8) |
| NOV2c | (SEQ ID NO: 10) |
| NOV2d | (SEQ ID NO: 12) |
| NOV2e | (SEQ ID NO: 14) |
| NOV2f | (SEQ ID NO: 16) |
| NOV2g | (SEQ ID NO: 18) |
| NOV2h | (SEQ ID NO: 20) |

Further analysis of the NOV2a protein yielded the following properties shown in Table 2C.

TABLE 2C
Protein Sequence Properties NOV2a

| SignalP | No Known Signal Sequence Predicted | | | |
|---|---|---|---|---|
| analysis: | | | | |
| PSORT II | PSG: a new signal peptide prediction method | | | |
| analysis: | N-region: length 11; pos. chg 1; neg. chg 1 | | | |
| | H-region: length 5; peak value −8.91 | | | |
| | PSG score: −13.31 | | | |
| | GvH: von Heijne's method for signal seq. recognition | | | |
| | GvH score (threshold: −2.1): −7.65 | | | |
| | possible cleavage site: between 53 and 54 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 3 | | | |
| | Number of TMS(s) for threshold 0.5: 1 | | | |
| | INTEGRAL | Likelihood = | −4.83 | Transmembrane 259-275 |
| | PERIPHERAL | Likelihood = | 1.54 (at 232) | |
| | ALOM score: −4.83 (number of TMSs: 1) | | | |
| | MTOP: Prediction of membrane topology (Hartmann et al.) | | | |
| | Center position for calculation: 266 | | | |
| | Charge difference: −3.5 C(−2.5)-N(1.0) | | | |
| | N >= C: N-terminal side will be inside | | | |
| | >>> membrane topology: type 2 (cytoplasmic tail 1 to 259) | | | |
| | MITDISC: discrimination of mitochondrial targeting seq | | | |
| | R content: | 7 | Hyd Moment(75): | 4.89 |
| | Hyd Moment(95): | 4.42 | G content: | 7 |
| | D/E content: | 2 | S/T content: | 8 |

```
Score: −1.94
Gavel: prediction of cleavage sites for mitochondrial preseq
R-2 motif at 104 LRA|PG
NUCDISC: discrimination of nuclear localization signals
pat4: none
pat7: none
bipartite: none
content of basic residues: 9.3%
NLS Score: −0.47
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: too long tail
Dileucine motif in the tail: found
LL at 81
LL at 82
LL at 83
LL at 237
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: nuclear
Reliability: 94.1
COIL: Lupas's algorithm to detect coiled-coil regions
total: 0 residues
Final Results (k = 9/23):
47.8%: nuclear
26.1%: mitochondrial
  8.7%: cytoplasmic
  4.3%: Golgi
  4.3%: plasma membrane
  4.3%: extracellular, including cell wall
  4.3%: peroxisomal
>> prediction for CG106951-01 is nuc (k = 23)
```

A search of the NOV2a protein against the Geneseq database, a proprietary database that contains sequences published in patents and patent publication, yielded several homologous proteins shown in Table 2D.

TABLE 2D
Geneseq Results for NOV2a

| | | NOV2a | | |
|---|---|---|---|---|
| | | Residues/ | Identities/ | |
| Geneseq | Protein/Organism/Length [Patent | Match | Similarities for the | Expect |
| Identifier | #, Date] | Residues | Matched Region | Value |
| AAE18212 | Human MOL4 protein - Homo | 1 . . . 1352 | 1352/1352 (100%) | 0.0 |
| | sapiens, 1352 aa. | 1 . . . 1352 | 1352/1352 (100%) | |
| | [WO200206339-A2, 24 JAN. 2002] | | | |
| AAG68293 | Human semaphorin G-like NHP | 202 . . . 1352 | 1150/1151 (99%) | 0.0 |
| | protein SEQ ID NO: 10 - Homo | 1 . . . 1151 | 1150/1151 (99%) | |
| | sapiens, 1151 aa. | | | |
| | [WO200188133-A2, 22 NOV. 2001] | | | |
| AAG68294 | Human semaphorin G-like NHP | 202 . . . 1352 | 1135/1151 (98%) | 0.0 |
| | protein SEQ ID NO: 12 - Homo | 1 . . . 1136 | 1135/1151 (98%) | |
| | sapiens, 1136 aa. | | | |
| | [WO200188133-A2, 22 NOV. 2001] | | | |
| AAG68290 | Human semaphorin G-like NHP | 260 . . . 1352 | 1092/1093 (99%) | 0.0 |
| | protein SEQ ID NO: 4 - Homo | 1 . . . 1093 | 1092/1093 (99%) | |
| | sapiens, 1093 aa. | | | |
| | [WO200188133-A2, 22 NOV. 2001] | | | |
| AAG68292 | Human semaphorin G-like NHP | 260 . . . 1352 | 1077/1093 (98%) | 0.0 |
| | protein SEQ ID NO: 8 - Homo | 1 . . . 1078 | 1077/1093 (98%) | |
| | sapiens, 1078 aa. | | | |
| | [WO200188133-A2, 22 NOV. 2001] | | | |

In a BLAST search of public sequence databases, the NOV2a protein was found to have homology to the proteins shown in the BLASTP data in Table 2E.

TABLE 2E
Public BLASTP Results for NOV2a

| | | NOV2a | | |
|---|---|---|---|---|
| Protein | | Residues/ | Identities/ | |
| Accession | | Match | Similarities for the | Expect |
| Number | Protein/Organism/Length | Residues | Matched Portion | Value |
| Q9P283 | Hypothetical protein KIAA1445 - | 151 . . . 1352 | 1202/1202 (100%) | 0.0 |
| | Homo sapiens (Human), 1202 aa | 1 . . . 1202 | 1202/1202 (100%) | |
| | (fragment). | | | |
| Q60519 | Semaphorin 5B precursor | 260 . . . 1352 | 1021/1093 (93%) | 0.0 |
| | (Semaphorin G) (Sema G) - Mus | 1 . . . 1093 | 1053/1093 (95%) | |
| | musculus (Mouse), 1093 aa. | | | |
| Q13591 | Semaphorin 5A precursor | 299 . . . 1336 | 616/1043 (59%) | 0.0 |
| | (Semaphorin F) (Sema F) - Homo | 30 . . . 1071 | 781/1043 (74%) | |
| | sapiens (Human), 1074 aa. | | | |
| Q62217 | Semaphorin 5A precursor | 299 . . . 1336 | 617/1046 (58%) | 0.0 |
| | (Semaphorin F) (Sema F) - Mus | 30 . . . 1074 | 776/1046 (73%) | |
| | musculus (Mouse), 1077 aa. | | | |
| Q8BXU8 | Sema domain - Mus musculus | 299 . . . 1109 | 507/811 (62%) | 0.0 |
| | (Mouse), 844 aa. | 30 . . . 839 | 632/811 (77%) | |

PFam analysis predicts that the NOV2a protein contains the domains shown in the Table 2F.

TABLE 2F
Domain Analysis of NOV2a

| | | Identities/ | |
|---|---|---|---|
| Pfam | | Similarities | Expect |
| Domain | NOV2a Match Region | for the Matched Region | Value |
| Sema | 327 . . . 738 | 217/491 (44%) | 7e−202 |
| | | 372/491 (76%) | |
| PSI | 756 . . . 803 | 18/67 (27%) | 2.5e−14 |
| | | 40/67 (60%) | |
| tsp_1 | 869 . . . 920 | 23/54 (43%) | 3.5e−12 |
| | | 38/54 (70%) | |
| tsp_1 | 927 . . . 971 | 17/53 (32%) | 4.3e−06 |
| | | 31/53 (58%) | |
| tsp_1 | 1058 . . . 1108 | 24/53 (45%) | 9.1e−11 |
| | | 34/53 (64%) | |
| tsp_1 | 1115 . . . 1165 | 23/53 (43%) | 5.9e−08 |
| | | 35/53 (66%) | |
| tsp_1 | 1170 . . . 1210 | 17/53 (32%) | 0.0034 |
| | | 27/53 (51%) | |

### Example 3

The NOV3 clone was analyzed, and the nucleotide and encoded polypeptide sequences are shown in Table 3A.

TABLE 3A
NOV3 Sequence Analysis

| | | |
|---|---|---|
| NOV3a, CG121295-01 | SEQ ID NO: 23 | 750 bp |
| DNA Sequence | ORF Start: ATG at 41 | ORF Stop: TGA at 701 |

TTCAGTTTGAACGGGAGGTTTTTGATCCCTTTTTTTCAGAATGGATTATTTGCTCATGATTTTCTCTCTGCTG
TTTGTGGCTTGCCAAGGAGCTCCAGAAACAGCAGTCTTAGGCGCTGAGCTCAGCGCGGTGGGTGAGAACGGCG
GGGAGAAACCCACTCCCAGTCCACCCTGGCGGCTCCGCCGGTCCAAGCGCTGCTCCTGCTCGTCCCTGATGGA
TAAAGAGTGTGTCTACTTCTGCCACCTGGACATCATTTGGGTCAACACTCCCGATTTCTTTCTCTCTTTGGAT
AATAGGCACGTTGTTCCGTATGGACTTGGAAGCCCTAGGTCCAAGAGAGCCTTGGAGAATTTACTTCCCACAA
AGGCAACAGACCGTGAGAATAGATGCCAATGTGCTAGCCAAAAAGACAAGAAGTGCTGGAATTTTTGCCAAGC
AGGAAAAGAACTCAGGGCTGAAGACATTATGGAGAAAGACTGGAATAATCATAAGAAAGGAAAAGACTGTTCC
AAGCTTGGGAAAAAGTGTATTTATCAGCAGTTAGTGAGAGGAAGAAAAATCAGAAGAAGTTCAGAGGAACACC
TAAGACAAACCAGGTCGGAGACCATGAGAAACAGCGTCAAATCATCTTTTCATGATCCCAAGCTGAAAGGCAA
GCCCTCCAGAGAGCGTTATGTGACCCACAACCGAGCACATTGGTGACAGACCTTCGGGGCCTGTCTGAAGCCA
TAGCCTCCACGGAGAGCCCT

| | | | |
|---|---|---|---|
| NOV3a, CG121295-01 | | | |
| Protein Sequence | SEQ ID NO: 24 | 220 aa | MW at 25403.9kD |

MDYLLMIFSLLFVACQGAPETAVLGAELSAVGENGGEKPTPSPPWRLRRSKRCSCSSLMDKECVYFCHLDIIW
VNTPDFFLSLDNRHVVPYGLGSPRSKRALENLLPTKATDRENRCQCASQKDKKCWNFCQAGKELRAEDIMEKD
WNNHKKGKDCSKLGKKCIYQQLVRGRKIRRSSEEHLRQTRSETMRNSVKSSFHDPKLKGKPSRERYVTHNRAH
W

Further analysis of the NOV3a protein yielded the following properties shown in Table 3B.

TABLE 3B
Protein Sequence Properties NOV3a

| SignalP | Cleavage site between residues 18 and 19 | | | |
|---|---|---|---|---|
| analysis: | | | | |
| PSORT II | PSG: a new signal peptide prediction method | | | |
| analysis: | N-region: length 2; pos. chg 0; neg. chg 1 | | | |
| | H-region: length 17; peak value 0.00 | | | |
| | PSG score: −4.40 | | | |
| | GvH: von Heijne's method for signal seq. recognition | | | |
| | GvH score (threshold: −2.1): 1.68 | | | |
| | possible cleavage site: between 17 and 18 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 1 | | | |
| | Number of TMS(s) for threshold 0.5: 0 | | | |
| | PERIPHERAL | Likelihood = 6.31 (at 67) | | |
| | ALOM score: −1.59 (number of TMSs: 0) | | | |
| | MITDISC: discrimination of mitochondrial targeting seq | | | |
| | R content: | 0 | Hyd Moment (75): | 4.56 |
| | Hyd Moment (95): | 7.21 | G content: | 1 |
| | D/E content: | 2 | S/T content: | 1 |

```
Score: −7.31
Gavel: prediction of cleavage sites for mitochondrial preseq
cleavage site motif not found
NUCDISC: discrimination of nuclear localization signals
pat4: none
pat7: PPWRLRR (3) at 43
pat7: PWRLRRS (4) at 44
pat7: PRSKRAL (5) at 96
bipartite: KKCIYQQLVRGRKIRRS at 161
content of basic residues: 18.6%
NLS Score: 1.05
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: nuclear
Reliability: 94.1
COIL: Lupas's algorithm to detect coiled-coil regions
total: 0 residues
Final Results (k = 9/23):
69.6%: nuclear
13.0%: mitochondrial
  8.7%: extracellular, including cell wall
  8.7%: cytoplasmic
>> prediction for CG121295-01 is nuc (k = 23)
```

A search of the NOV3a protein against the Geneseq database, a proprietary database that contains sequences published in patents and patent publication, yielded several homologous proteins shown in Table 3C.

TABLE 3C
Geneseq Results for NOV3a

| | | NOV3a | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| ABU03518 | Angiogenesis-associated human protein | 1 . . . 220 | 211/220 (95%) | e−125 |
| | sequence #63 - Homo sapiens, 212 aa. | 1 . . . 212 | 212/220 (95%) | |
| | [WO200279492-A2, 10 OCT. 2002] | | | |
| ABP65215 | Hypoxia-regulated protein #89 - Homo | 1 . . . 220 | 211/220 (95%) | e−125 |
| | sapiens, 212 aa. [WO200246465-A2, | 1 . . . 212 | 212/220 (95%) | |
| | 13 JUN. 2002] | | | |
| AAG64862 | Heart muscle cell differentiation related | 1 . . . 220 | 211/220 (95%) | e−125 |
| | protein SEQ ID NO: 65 - Homo | 1 . . . 212 | 212/220 (95%) | |
| | sapiens, 212 aa. [WO200148151-A1, | | | |
| | 05 JUL. 2001] | | | |
| AAB99933 | Human ET1 protein sequence SEQ ID | 1 . . . 220 | 211/220 (95%) | e−125 |
| | NO: 65 - Homo sapiens, 212 aa. | 1 . . . 212 | 212/220 (95%) | |
| | [WO200148150-A1, 05 JUL. 2001] | | | |
| AAB00197 | Preproendothelin-1 - Homo sapiens, | 1 . . . 220 | 211/220 (95%) | e−125 |
| | 212 aa. [WO200055314-A2, | 1 . . . 212 | 212/220 (95%) | |
| | 21 SEP. 2000] | | | |

In a BLAST search of public sequence databases, the NOV3a protein was found to have homology to the proteins shown in the BLASTP data in Table 3D.

TABLE 3D
Public BLASTP Results for NOV3a

| | | NOV3a | | |
|---|---|---|---|---|
| Protein | | Residues/ | Identities/ | |
| Accession | | Match | Similarities for the | Expect |
| Number | Protein/Organism/Length | Residues | Matched Portion | Value |
| P05305 | Endothelin-1 precursor (ET-1) - | 1 . . . 220 | 211/220 (95%) | e−124 |
| | Homo sapiens (Human), 212 aa. | 1 . . . 212 | 212/220 (95%) | |
| P17322 | Endothelin-1 precursor (ET-1) - | 1 . . . 219 | 148/220 (67%) | 3e−80 |
| | Bos taurus (Bovine), 202 aa. | 1 . . . 202 | 167/220 (75%) | |
| P09558 | Endothelin-1 precursor (ET-1) - Sus | 1 . . . 219 | 145/221 (65%) | 7e−78 |
| | scrofa (Pig), 203 aa. | 1 . . . 203 | 168/221 (75%) | |
| P22387 | Endothelin-1 precursor (ET-1) - | 1 . . . 219 | 147/220 (66%) | 1e−77 |
| | Mus musculus (Mouse), 202 aa. | 1 . . . 202 | 165/220 (74%) | |
| Q9BG76 | Preproendothelin-1 - Ovis aries | 1 . . . 219 | 142/220 (64%) | 9e−76 |
| | (Sheep), 202 aa. | 1 . . . 202 | 164/220 (74%) | |

PFam analysis predicts that the NOV3a protein contains the domains shown in the Table 3E.

TABLE 3E
Domain Analysis of NOV3a

| | | Identities/ | |
|---|---|---|---|
| | NOV3a Match | Similarities | Expect |
| Pfam Domain | Region | for the Matched Region | Value |
| endothelin | 48 . . . 78 | 26/31 (84%) | 8.6e−20 |
| | | 31/31 (100%) | |

### Example 4

The NOV4 clone was analyzed, and the nucleotide and encoded polypeptide sequences are shown in Table 4A.

TABLE 4A
NOV4 Sequence Analysis

| | | |
|---|---|---|
| NOV4a, CG124756-01 | SEQ ID NO: 25 | 1076 bp |
| DNA Sequence | ORF Start: ATG at 75 | ORF Stop: TGA at 834 |

GGCTCCTGGTCCCACTGCTGCTCAGCCCAGTGGCCTCACAGGACACCAGCTTCCCAGGAGGCGTCTGACACAG
TATGATGATGAAGATCCCATGGGGCAGCATCCCAGTACTGATGTTGCTCCTGCTCCTGGGCCTAATCGATATC
TCCCAGGCCCAGCTCAGCTGCACCGGGCCCCCAGCCATCCCTGGCATCCCGGGTATCCCTGGGACACCTGGCC
CCGATGGCCAACCTGGGACCCCAGGGATAAAAGGAGAGAAAGGGCTTCCAGGGCTGGCTGGAGACCATGGTGA
GTTCGGAGAGAAGGGAGACCCAGGGATTCCTGGGAATCCAGGAAAAGTCGGCCCCAAGGGCCCCATGGGCCCT
AAAGGTGGCCCAGGGGCCCCTGGAGCCCCAGGCCCCAAAGGTGAATCGGGAGACTACAAGGCCACCCAGAAAA
TCGCCTTCTCTGCCACAAGAACCATCAACGTCCCCCTGCGCCGGGACCAGACCATCCGCTTCGACCACGTGAT
CACCAACATGAACAACAATTATGAGCCCCGCAGTGGCAAGTTCACCTGCAAGGTGCCCGGTCTCTACTACTTC
ACCTACCACGCCAGCTCTCGAGGGAACCTGTGCGTGAACCTCATGCGTGGCCGGGAGCGTGCACAGAAGGTGG
TCACCTTCTGTGACTATGCCTACAACACCTTCCAGGTCACCACCGGTGGCATGGTCCTCAAGCTGGAGCAGGG
GGAGAACGTCTTCCTGCAGGCCACCGACAAGAACTCACTACTGGGCATGGAGGGTGCCAACAGCATCTTTTCC
GGGTTCCTGCTCTTTCCAGATATGGAGGCCTGACCTGTGGGCTGCTTCACATCCACCCCGGCTCCCCCTGCCA
GCAACGCTCACTCTACCCCCAACACCACCCCTTGCCCAGCCAATGCACACAGTAGGGCTTGGTGAATGCTGCT
GAGTGAATGAGTAAATAAACTCTTCAAGGCCAAGGGAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAA

| | | | |
|---|---|---|---|
| NOV4a, CG124756-01 | | | |
| Protein Sequence | SEQ ID NO: 26 | 253 aa | MW at 26721.5kD |

MMMKIPWGSIPVLMLLLLLGLIDISQAQLSCTGPPAIPGIPGIPGTPGPDGQPGTPGIKGEKGLPGLAGDHGE
FGEKGDPGIPGNPGKVGPKGPMGPKGGPGAPGAPGPKGESGDYKATQKIAFSATRTINVPLRRDQTIRFDHVI
TNMNNNYEPRSGKFTCKVPGLYYFTYHASSRGNLCVNLMRGRERAQKVVTFCDYAYNTFQVTTGGMVLKLEQG
ENVFLQATDKNSLLGMEGANSIFSGFLLFPDMEA

| | | |
|---|---|---|
| NOV4b, CG124756-02 | SEQ ID NO: 27 | 816 bp |
| DNA Sequence | ORF Start: ATG at 48 | ORF Stop: TGA at 807 |

GTGGTAACCTTCACATTGTCTTCTCCACAGGAGGCGTCTGACACAGTATGATGATGAAGATCCCATGGGGCAG
CATCCCAGTACTGATGTTGCTCCTGCTCCTGGGCCTAATCGATATCTCCCAGGCCCAGCTCAGCTGCACCGGG
CCCCCAGCCATCCCTGGCATCCCGGGTATCCCTGGGACACCTGGCCCCGATGGCCAACCTGGGACCCCAGGGA
TAAAAGGAGAGAAAGGGCTTCCAGGGCTGGCTGGAGACCATGGTGAGTTCGGAGAGAAGGGAGACCCAGGGAT
TCCTGGGAATCCAGGAAAAGTCGGCCCCAAGGGCCCCATGGGCCCTAAAGGTGGCCCAGGGGCCCCTGGAGCC
CCAGGCCCCAAAGGTGAATCGGGAGACTACAAGGCCACCCAGAAAATCGCCTTCTCTGCCACAAGAACCATCA
ACGTCCCCCTGCGCCGGGACCAGACCATCCGCTTCGACCACGTGATCACCAACATGAACAACAATTATGAGCC
CCGCAGTGGCAAGTTCACCTGCAAGGTGCCCGGTCTCTACTACTTCACCTACCACGCCAGCTCTCGAGGGAAC
CTGTGCGTGAACCTCATGCGTGGCCGGGAGCGTGCACAGAAGGTGGTCACCTTCTGTGACTATGCCTACAACA
CCTTCCAGGTCACCACCGGTGCCATGGTCCTCAAGCTGGAGCAGGGGGAGAACGTCTTCCTGCAGGCCACCGA
CAAGAACTCACTACTGGGCATGGAGGGTGCCAACAGCATCTTTTCCGGGTTCCTGCTCTTTCCAGATATGGAG
GCCTGACCTGTGG

| | | | |
|---|---|---|---|
| NOV4b, CG124756-02 | | | |
| Protein Sequence | SEQ ID NO: 28 | 253 aa | MW at 26721.5kD |

MMMKIPWGSIPVLMLLLLLGLIDISQAQLSCTGPPAIPGIPGIPGTPGPDGQPGTPGIKGEKGLPGLAGDHGE
FGEKGDPGIPGNPGKVGPKGPMGPKGGPGAPGAPGPKGESGDYKATQKIAFSATRTINVPLRRDQTIRFDHVI
TNMNNNYEPRSGKFTCKVPGLYYFTYHASSRGNLCVNLMRGRERAQKVVTFCDYAYNTFQVTTGGMVLKLEQG
ENVFLQATDKNSLLGMEGANSIFSGFLLFPDMEA

| | | |
|---|---|---|
| | SEQ ID NO: 29 | 1076 bp |
| NOV4c, SNP13382475 of | ORF Start: ATG at 75 | ORF Stop: TGA at 834 |
| CG124756-01, DNA Sequence | SNP Pos: 302 | SNP Change: G to T |

GGCTCCTGGTCCCACTGCTGCTCAGCCCAGTGGCCTCACAGGACACCAGCTTCCCAGGAGGCGTCTGACACAG
TATGATGATGAAGATCCCATGGGGCAGCATCCCAGTACTGATGTTGCTCCTGCTCCTGGGCCTAATCGATATC
TCCCAGGCCCAGCTCAGCTGCACCGGGCCCCCAGCCATCCCTGGCATCCCGGGTATCCCTGGGACACCTGGCC
CCGATGGCCAACCTGGGACCCCAGGGATAAAAGGAGAGAAAGGGCTTCCAGGGCTGGCTGGAGACCATGGTGA
GTTCGGAGATAAGGGAGACCCAGGGATTCCTGGGAATCCAGGAAAAGTCGGCCCCAAGGGCCCCATGGGCCCT
AAAGGTGGCCCAGGGGCCCCTGGAGCCCCAGGCCCCAAAGGTGAATCGGGAGACTACAAGGCCACCCAGAAAA
TCGCCTTCTCTGCCACAAGAACCATCAACGTCCCCCTGCGCCGGGACCAGACCATCCGCTTCGACCACGTGAT
CACCAACATGAACAACAATTATGAGCCCCGCAGTGGCAAGTTCACCTGCAAGGTGCCCGGTCTCTACTACTTC
ACCTACCACGCCAGCTCTCGAGGGAACCTGTGCGTGAACCTCATGCGTGGCCGGGAGCGTGCACAGAAGGTGG
TCACCTTCTGTGACTATGCCTACAACACCTTCCAGGTCACCACCGGTGGCATGGTCCTCAAGCTGGAGCAGGG
GGAGAACGTCTTCCTGCAGGCCACCGACAAGAACTCACTACTGGGCATGGAGGGTGCCAACAGCATCTTTTCC
GGGTTCCTGCTCTTTCCAGATATGGAGGCCTGACCTGTGGGCTGCTTCACATCCACCCCGGCTCCCCCTGCCA
GCAACGCTCACTCTACCCCCAACACCACCCCTTGCCCAGCCAATGCACACAGTAGGGCTTGGTGAATGCTGCT
GAGTGAATGAGTAAATAAACTCTTCAAGGCCAAGGGAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAA

| | | | |
|---|---|---|---|
| NOV4c, SNP13382475 of | SEQ ID NO: 30 | | MW at 26707.5kD |
| CG124756-01, Protein Sequence | SNP Pos: 76 | 253 aa | SNP Change: Glu to Asp |

MMMKIPWGSIPVLMLLLLLGLIDISQAQLSCTGPPAIPGIPGIPGTPGPDGQPGTPGIKGEKGLPGLAGDHGE
FGDKGDPGIPGNPGKVGPKGPMGPKGGPGAPGAPGPKGESGDYKATQKIAFSATRTINVPLRRDQTIRFDHVI
TNMNNNYEPRSGKFTCKVPGLYYFTYHASSRGNLCVNLMRGRERAQKVVTFCDYAYNTFQVTTGGMVLKLEQG
ENVFLQATDKNSLLGMEGANSIFSGFLLFPDMEA

| | | |
|---|---|---|
| | SEQ ID NO: 31 | 1076 bp |
| NOV4d, SNP13382476 of | ORF Start: ATG at 75 | ORF Stop: TGA at 834 |
| CG124756-01, DNA Sequence | SNP Pos: 433 | SNP Change: A to G |

GGCTCCTGGTCCCACTGCTGCTCAGCCCAGTGGCCTCACAGGACACCAGCTTCCCAGGAGGCGTCTGACACAG
TATGATGATGAAGATCCCATGGGGCAGCATCCCAGTACTGATGTTGCTCCTGCTCCTGGGCCTAATCGATATC
TCCCAGGCCCAGCTCAGCTGCACCGGGCCCCCAGCCATCCCTGGCATCCCGGGTATCCCTGGGACACCTGGCC
CCGATGGCCAACCTGGGACCCCAGGGATAAAAGGAGAGAAAGGGCTTCCAGGGCTGGCTGGAGACCATGGTGA
GTTCGGAGAGAAGGGAGACCCAGGGATTCCTGGGAATCCAGGAAAAGTCGGCCCCAAGGGCCCCATGGGCCCT
AAAGGTGGCCCAGGGGCCCCTGGAGCCCCAGGCCCCAAAGGTGAATCGGGAGACTACAAGGCCACCCGGAAAA
TCGCCTTCTCTGCCACAAGAACCATCAACGTCCCCCTGCGCCGGGACCAGACCATCCGCTTCGACCACGTGAT
CACCAACATGAACAACAATTATGAGCCCCGCAGTGGCAAGTTCACCTGCAAGGTGCCCGGTCTCTACTACTTC
ACCTACCACGCCAGCTCTCGAGGGAACCTGTGCGTGAACCTCATGCGTGGCCGGGAGCGTGCACAGAAGGTGG
TCACCTTCTGTGACTATGCCTACAACACCTTCCAGGTCACCACCGGTGGCATGGTCCTCAAGCTGGAGCAGGG
GGAGAACGTCTTCCTGCAGGCCACCGACAAGAACTCACTACTGGGCATGGAGGGTGCCAACAGCATCTTTTCC
GGGTTCCTGCTCTTTCCAGATATGGAGGCCTGACCTGTGGGCTGCTTCACATCCACCCCGGCTCCCCCTGCCA
GCAACGCTCACTCTACCCCCAACACCACCCCTTGCCCAGCCAATGCACACAGTAGGGCTTGGTGAATGCTGCT
GAGTGAATGAGTAAATAAACTCTTCAAGGCCAAGGGAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAA

| | | | |
|---|---|---|---|
| NOV4d, SNP13382476 of | SEQ ID NO: 32 | | MW at 26749.6kD |
| CG124756-01, Protein Sequence | SNP Pos: 120 | 253 aa | SNP Change: Gln to Arg |

MMMKIPWGSIPVLMLLLLLGLIDISQAQLSCTGPPAIPGIPGIPGTPGPDGQPGTPGIKGEKGLPGLAGDHGE
FGEKGDPGIPGNPGKVGPKGPMGPKGGPGAPGAPGPKGESCDYKATRKIAFSATRTINVPLRRDQTIRFDHVI
TNMNNNYEPRSGKFTCKVPGLYYFTYHASSRGNLCVNLMRGRERAQKVVTFCDYAYNTFQVTTGGMVLKLEQG
ENVFLQATDKNSLLGMEGANSIFSGFLLFPDMEA

A ClustalW comparison of the above protein sequences yields the following sequence alignment shown in Table 4B.

TABLE 4B

| Comparison of the NOV4 protein sequences. | |
|---|---|
| NOV4a | MMMKIPWGSIPVLMLLLLLGLIDISQAQLSCTGPPAIPGIPGIPGTPGPDGQPGTPGIKG |
| NOV4b | MMMKIPWGSIPVLMLLLLLGLIDISQAQLSCTGPPAIPGIPGIPGTPGPDGQPGTPGIKG |
| NOV4a | EKGLPGLAGDHGEFGEKGDPGIPGNPGKVGPKGPMGPKGGPGAPGAPGPKGESGDYKATQ |
| NOV4b | EKGLPGLAGDHGEFGEKGDPGIPGNPGKVGPKGPMGPKGGPGAPGAPGPKGESGDYKATQ |
| NOV4a | KIAFSATRTINVPLRRDQTIRFDHVITNMNNNYEPRSGKFTCKVPGLYYFTYHASSRGNL |
| NOV4b | KIAFSATRTINVPLRRDQTIRFDHVITNMNNNYEPRSGKFTCKVPGLYYFTYHASSRGNL |
| NOV4a | CVNLMRGRERAQKVVTFCDYAYNTFQVTTGGMVLKLEQGENVFLQATDKNSLLGMEGANS |
| NOV4b | CVNLMRGRERAQKVVTFCDYAYNTFQVTTGGMVLKLEQGENVFLQATDKNSLLGMEGANS |
| NOV4a | IFSGFLLFPDMEA |
| NOV4b | IFSGFLLFPDMEA |
| NOV4a | (SEQ ID NO: 26) |
| NOV4b | (SEQ ID NO: 28) |

Further analysis of the NOV4a protein yielded the following properties shown in Table 4C.

TABLE 4C
Protein Sequence Properties NOV4a

| SignalP | Cleavage site between residues 28 and 29 | | | |
|---|---|---|---|---|
| analysis: | | | | |
| PSORT II | PSG: a new signal peptide prediction method | | | |
| analysis: | N-region: length 4; pos. chg 1; neg. chg 0 | | | |
| | H-region: length 18; peak value 11.91 | | | |
| | PSG score: 7.51 | | | |
| | GvH: von Heijne's method for signal seq. recognition | | | |
| | GvH score (threshold: −2.1): 4.21 | | | |
| | possible cleavage site: between 27 and 28 | | | |
| | >>> Seems to have a cleavable signal peptide (1 to 27) | | | |
| | ALOM: Klein et al's method for TM region allocation | | | |
| | Init position for calculation: 28 | | | |
| | Tentative number of TMS(s) for the threshold 0.5: 0 | | | |
| | number of TMS(s) . . . fixed | | | |
| | PERIPHERAL | Likelihood = 2.60 (at 232) | | |
| | ALOM score: 2.60 (number of TMSs: 0) | | | |
| | MTOP: Prediction of membrane topology (Hartmann et al.) | | | |
| | Center position for calculation: 13 | | | |
| | Charge difference: −3.0 C(−1.0)-N(2.0) | | | |
| | N >= C: N-terminal side will be inside | | | |
| | MITDISC: discrimination of mitochondrial targeting seq | | | |
| | R content: | 0 | Hyd Moment(75): | 6.93 |
| | Hyd Moment(95): | 5.45 | G content: | 2 |
| | D/E content: | 1 | S/T content: | 1 |

Score: −5.61
Gavel: prediction of cleavage sites for mitochondrial preseq
cleavage site motif not found
NUCDISC: discrimination of nuclear localization signals
pat4: none
pat7: none
bipartite: none
content of basic residues: 9.5%
NLS Score: −0.47
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 76.7
COIL: Lupas's algorithm to detect coiled-coil regions
total: 0 residues
Final Results (k = 9/23):
22.2%: extracellular, including cell wall
22.2%: vacuolar
22.2%: mitochondrial
22.2%: endoplasmic reticulum
11.1%: Golgi
>> prediction for CG124756-01 is exc (k = 9)

A search of the NOV4a protein against the Geneseq database, a proprietary database that contains sequences published in patents and patent publication, yielded several homologous proteins shown in Table 4D.

TABLE 4D
Geneseq Results for NOV4a

| | | NOV4a | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| AAM40607 | Human polypeptide SEQ ID NO 5538 - | 1 . . . 253 | 253/253 (100%) | e−151 |
| | Homo sapiens, 255 aa. | 3 . . . 255 | 253/253 (100%) | |
| | [WO200153312-A1, 26 JUL. 2001] | | | |
| AAM38821 | Human polypeptide SEQ ID NO 1966 - | 1 . . . 253 | 253/253 (100%) | e−151 |
| | Homo sapiens, 253 aa. | 1 . . . 253 | 253/253 (100%) | |
| | [WO200153312-A1, 26 JUL. 2001] | | | |
| ABB57231 | Mouse ischaemic condition related | 3 . . . 253 | 201/253 (79%) | e−117 |
| | protein sequence SEQ ID NO: 599 - | 1 . . . 253 | 218/253 (85%) | |
| | Mus musculus, 253 aa. | | | |
| | [WO200188188-A2, 22 NOV. 2001] | | | |
| AAU32411 | Novel human secreted protein #2902 - | 1 . . . 248 | 203/267 (76%) | e−103 |
| | Homo sapiens, 309 aa. | 3 . . . 269 | 212/267 (79%) | |
| | [WO200179449-A2, 25 OCT. 2001] | | | |
| AAU30709 | Novel human secreted protein #1200 - | 23 . . . 248 | 195/243 (80%) | e−102 |
| | Homo sapiens, 287 aa. | 2 . . . 244 | 199/243 (81%) | |
| | [WO200179449-A2, 25 OCT. 2001] | | | |

In a BLAST search of public sequence databases, the NOV4a protein was found to have homology to the proteins shown in the BLASTP data in Table 4E.

TABLE 4E
Public BLASTP Results for NOV4a

| | | NOV4a | | |
|---|---|---|---|---|
| Protein | | Residues/ | Identities/ | |
| Accession | | Match | Similarities for the | Expect |
| Number | Protein/Organism/Length | Residues | Matched Portion | Value |
| C1HUQB | complement subcomponent C1q | 1 . . . 253 | 253/253 (100%) | e−151 |
| | chain B precursor [validated] - | 1 . . . 253 | 253/253 (100%) | |
| | human, 253 aa. | | | |
| P02746 | Complement C1q subcomponent, B | 3 . . . 253 | 251/251 (100%) | e−150 |
| | chain precursor - Homo sapiens | 1 . . . 251 | 251/251 (100%) | |
| | (Human), 251 aa. | | | |
| P14106 | Complement C1q subcomponent, B | 3 . . . 253 | 201/253 (79%) | e−117 |
| | chain precursor - Mus musculus | 1 . . . 253 | 219/253 (86%) | |
| | (Mouse), 253 aa. | | | |
| I49560 | complement C1q B chain precursor - | 3 . . . 253 | 201/253 (79%) | e−117 |
| | mouse, 253 aa. | 1 . . . 253 | 218/253 (85%) | |
| P31721 | Complement C1q subcomponent, B | 3 . . . 252 | 197/252 (78%) | e−115 |
| | chain precursor - Rattus norvegicus | 1 . . . 252 | 217/252 (85%) | |
| | (Rat), 253 aa. | | | |

PFam analysis predicts that the NOV4a protein contains the domains shown in the Table 4F.

TABLE 4F
Domain Analysis of NOV4a

| | | Identities/ | |
|---|---|---|---|
| | NOV4a Match | Similarities | Expect |
| Pfam Domain | Region | for the Matched Region | Value |
| Collagen | 51 . . . 110 | 35/60 (58%) | 8.7e−09 |
| | | 45/60 (75%) | |
| C1q | 123 . . . 247 | 69/138 (50%) | 2.4e−72 |
| | | 124/138 (90%) | |

### Example 5

The NOV5 clone was analyzed, and the nucleotide and encoded polypeptide sequences are shown in Table 5A.

Table 5A
NOV5 Sequence Analysis

| | | |
|---|---|---|
| NOV5a, CG50353-01 | SEQ ID NO: 33 | 1628 bp |
| DNA Sequence | ORF Start: ATG at 1 | ORF Stop: TGA at 1048 |

ATGAACCGGAAAGCGCGGCGCTGCCTGGGCCACCTCTTTCTCAGCCTGGGCATGGTCTGTCTCCTAGCATGTG
GCTTCTCCTCAGTGGTAGCTCTGGGCGCAACGGTCATCTGTAACAAGATCCCAGGCCTGGCTCCCAGACAGCG
GGCGATCTGCCAGAGCCGGCCCGACGCCATCATCGTCATAGGAGAAGGCTCACAAATGGGCCTGGACGAGTGT
CAGTTTCAGTTCCGCAATGGCCGCTGGAACTGCTCTGCACTGGGAGAGCGCACCGTCTTCGGGAAGGAGCTCA
AAGTGGGGAGCCGGGACGGTGCGTTCACCTACGCCATCATTGCCGCCGGCGTGGCCCACGCCATCACAGCTGC
CTGTACCCATGGCAACCTGAGCGACTGTGGCTGCGACAAAGAGAAGCAAGGCCAGTACCACCGGGACGAGGGC
TGGAAGTGGGGTGGCTGCTCTGCCGACATCCGCTACGGCATCGGCTTCGCCAAGGTCTTCGTGGACGCTCGGG
AGATCATGAAGAACGCGCGGCGCCTCATGAACCTGCATAACAATGAGGCCGGCAGGAAGGTTCTAGAGGACCG
GATGCAGCTGGAGTGCAAGTGCCACGGCGTGTCTGGCTCCTGCACCACCAAAACCTGCTGGACCACGCTGCCC
AAGTTCCGAGAGGTGGGCCACCTGCTGAAGGAGAAGTACAACGCGGCCGTGCAGGTGGAGGTGGTGCGGGCCA
GCCGTCTGCGGCAGCCCACCTTCCTGCGCATCAAACAGCTGCGCAGCTATCGCAAGCCCATGAAGACGGACCT
GGTGTACATCGAGAAGTCGCCCAACTACTGCGAGGAGGACCCGGTGACCGGCAGTGTGGGCACGCAGGGCCGC
GCCTGCAACAAGACGGCTCCCCAGGCCAGCGGCTGTGACCTCATGTGCTGTGGGCGTGGCTACAACACCCACC
AGTACGCCCGCGTGTGGCAGTGCAACTGTAAGTTCCACTGGTGCTGCTATGTCAAGTGCAACACGTGCAGCGA
GCGCACGGAGATGTACACGTGCAAGTGAGCCCCGTGTGCACACCACCCTCCCGCTGCAAGTCAGATTGCTGGG
AGGACTGGACCGTTTCCAAGCTGCGGGCTCCCTGGCAGGATGCTGAGCTTGTCTTTTCTGCTGAGGAGGGTAC
TTTTCCTGGGTTTCCTGCAGGCATCCGTGGGGGAAAAAAAATCTCTCAGAGCCCTCAACTATTCTGTTCCACA
CCCAATGCTGCTCCACCCTCCCCCAGACACAGCCCAGGTCCCTCCGCGGCTGGAGCGAAGCCTTCTGCAGCAG
GAACTCTGGACCCCTGGGCCTCATCACAGCAATATTTAACAATTTATTCTGATAAAAATAATATTAATTTATT
TAATTAAAAAGAATTCTTCCACCTCGTCGGGATCCGTTTTCTGCAATCAAAGTGGACTGCTTGCTTTCCTAGC
AGGATGATTTTGTTGCTAGGACAAGGAGCCGTGTAGAAGTGTACATAACTATTCTTTATGCAGATATTTCTAC
TAGCTGATTTTGCAGGTACCCACCTTGCAGCACTAGATGTTTAAGTACAAGAGGAGACATCTTTTATGCATAT
ATAGATATACACACACGAAAAA

| | | | |
|---|---|---|---|
| NOV5a, CG50353-01 | | | |
| Protein Sequence | SEQ ID NO: 34 | 349 aa | MW at 38980.7kD |

MNRKARRCLGHLFLSLGMVCLLACGFSSVVALGATVICNKIPGLAPRQRAICQSRPDAIIVIGEGSQMGLDEC
QFQFRNGRWNCSALGERTVFGKELKVGSRDGAFTYAIIAAGVAHAITAACTHGNLSDCGCDKEKQGQYHRDEG
WKWGGCSADIRYGIGFAKVFVDAREIMKNARRLMNLHNNEAGRKVLEDRMQLECKCHGVSGSCTTKTCWTTLP
KFREVGHLLKEKYNAAVQVEVVRASRLRQPTFLRIKQLRSYRKPMKTDLVYIEKSPNYCEEDPVTGSVGTQGR
ACNKTAPQASGCDLMCCGRGYNTHQYARVWQCNCKFHWCCYVKCNTCSERTEMYTCK

| | | |
|---|---|---|
| NOV5b, 228753443 | SEQ ID NO: 35 | 966 bp |
| DNA Sequence | ORF Start: at 1 | ORF Stop: end of sequence |

AGATCTCTGGGCGCAACGGTCATCTGTAACAAGATCCCAGGCCTGGCTCCCAGACAGCGGGCGATCTGCCAGA
GCCGGCCCGACGCCATCATCGTCATAGGAGAAGGCTCACAAATGGGCCTGGACGAGTGTCAGTTTCAGTTCCG
CAATGGCCGCTGGAACTGCTCTGCACTGGGAGAGCGCACCGTCTTCGGGAAGGAGCTCAAAGTGGGGAGCCGG
GAGGCTGCATTCACCTACGCCATCATTGCCGCCGGCGTGGCCCACGCCATCACAGCTGCCTGTACCCAGGGCA
ACCTGAGCGACTGTGGCTGCGACAAAGAGAAGCAAGGCCAGTACCACCGGGACGAGGGCTGGAAGTGGGGTGG
CTGCTCCGCCGACATCCGCTACGGCATCGGCTTCGCCAAGGTCTTTGTGGATGCCCGGGAGATCAAGCAGAAT
GCCCGGACTCTCATGAACTTGCACAACAACGAGGCAGGCCGAAAGATCCTGGAGGAGAACATGAAGCTGGAAT
GTAAGTGCCACGGCGTGTCAGGCTCGTGCACCACCAAGACGTGCTGGACCACACTGCCACAGTTTCGGGAGCT
GGGTACGTGCTCAAGGACAAGTACAACGAGGCCGTTCACGTGGAGCCTGTGCGTGCCAGCCGCAACAAGCGG
CCCACCTTCCTGAAGATCAAGAAGCCACTGTCGTACCGCAAGCCCATGGACACGGACCTGGTGTACATCGAGA
AGTCGCCCAACTACTGCGAGGAGGACCCGGTGACCGGCAGTGTGGGCACCCAGGGCCGCGCCTGCAACAAGAC
GGCTCCCCAGGCCAGCGGCTGTGACCTCATGTGCTGTGGGCGTGGCTACAACACCCACCAGTACGCCCGCGTG
TGGCAGTGCAACTGTAAGTTCCACTGGTGCTGCTATGTCAAGTGCAACACGTGCAGCGAGCGCACGGAGATGT
ACACGTGCAAGCTCGAG

| | | | |
|---|---|---|---|
| NOV5b, 228753443 | | | |
| Protein Sequence | SEQ ID NO: 36 | 322 aa | MW at 36054.9kD |

RSLGATVICNKIPGLAPRQRAICQSRPDAIIVIGEGSQMGLDECQFQFRNGRWNCSALGERTVFGKELKVGSR
EAAFTYAIIAAGVAHAITAACTQGNLSDCGCDKEKQGQYHRDEGWKWGGCSADIRYGIGFAKVFVDAREIKQN
ARTLMNLHNNEAGRKILEENMKLECKCHGVSGSCTTKTCWTTLPQFRELGYVLKDKYNEAVHVEPVRASRNKR
PTFLKIKKPLSYRKPMDTDLVYIEKSPNYCEEDPVTGSVGTQGRACNKTAPQASGCDLMCCGRGYNTHQYARV
WQCNCKFHWCCYVKCNTCSERTEMYTCKLE

| | | |
|---|---|---|
| NOV5c, 169475673 | SEQ ID NO: 37 | 966 bp |
| DNA Sequence | ORF Start: at 1 | ORF Stop: end of sequence |

AGATCTCTGGGCGCAACGGTCATCTGTAACAAGATCCCAGGCCTGGCTCCCAGACAGCGGGCGATCTGCCAGA
GCCGGCCCGACGCCATCATCGTCATAGGAGAAGGCTCACAAATGGGCCTCGACGAGTGTCAGTTTCAGTTCCG
CAATGGCCGCTGGAACTGCTCTGCACTGGGAGAGCGCACCGTCTTCGGGAAGGAGCTCAAAGTGGGGAGCCGG
GAGGCTGCGTTCACCTACGCCATCATTGCCGCCGGCGTGGCCCACGCCATCACAGCTGCCTGTACCCAGGGCA
ACCTGAGCGACTGTGGCTGCGACAAAGAGAAGCAAGGCCAGTACCACCGGGACGAGGGCTGGAAGTGGGGTGG
CTGCTCTGCCGACATCCGCTACGGCATCGGCTTCGCCAAGGTCTTTGTGGATGCCCGGGAGATCAAGCAGAAT
GCCCGGACTCTCATGAACTTGCACAACAACGAGGCAGGCCGAAAGATCCTGGAGGAGAACATGAAGCTGGAAT
GTAAGTGCCACGGCGTGTCAGGCTCGTGCACCACCAAGACGTGCTGGACCACACTGCCACAGTTTCGGGAGCT
GGGCTACGTGCTCAAGGACAAGTACAACGAGGCCGTTCACGTGGAGCCTGTGCGTGCCAGCCGCAACAAGCGG
CCCACCTTCCTGAAGATCAAGAAGCCACTGTCGTACCGCAAGCCCATGGACACGGACCTGGTGTACATCGAGA
AGTCGCCCAACTACTGCGAGGAGGACCCGGTGACCGGCAGTGTGGGCACCCAGGGCCGCGCCTGCAACAAGAC
GGCTCCCCAGGCCAGCGGCTGTGACCTCATGTGCTGTGGGCGTGGCTACAACACCCACCAGTACGCCCGCGTG
TGGCAGTGCAACTGTAAGTTCCACTGGTGCTGCTATGTCAAGTGCAACACGTGCAGCGAGCGCACGGAGATGT
ACACGTGCAAGCTCGAG

| | | | |
|---|---|---|---|
| NOV5c, 169475673 | | | |
| Protein Sequence | SEQ ID NO: 38 | 322 aa | MW at 36054.9kD |

RSLGATVICNKIPGLAPRQRAICQSRPDAIIVIGEGSQMGLDECQFQFRNGRWNCSALGERTVFGKELKVGSR
EAAFTYAIIAAGVAHAITAACTQGNLSDCGCDKEKQGQYHRDEGWKWGGCSADIRYGIGFAKVFVDAREIKQN
ARTLMNLHNNEAGRKILEENMKLECKCHGVSGSCTTKTCWTTLPQFRELGYVLKDKYNEAVHVEPVRASRNKR
PTFLKIKKPLSYRKPMDTDLVYIEKSPNYCEEDPVTGSVGTQGRACNKTAPQASGCDLMCCGRGYNTHQYARV
WQCNCKFHWCCYVKCNTCSERTEMYTCKLE

| | | |
|---|---|---|
| NOV5d, 228753459 | SEQ ID NO: 39 | 966 bp |
| DNA Sequence | ORF Start: at 1 | ORF Stop: end of sequence |

AGATCTCTGGGCGCAACGGTCATCTGTAACAAGATCCCAGGCCTGGCTCCCAGACAGCGGGCGATCTGCCAGA
GCCGGCCCGACGCCATCATCGTCATAGGAGAAGGCTCACAAATGGGCCTGGACGAGTGTCAGTTTCAGTTCCG
CAATGGCCGCTGGAACTGCTCTGCACTGGGAGAGCGCACCGTCTTCGGGAAGGAGCTCAAAGTGGGGAGCCGG
GAGGCTGCATTCACCTACGCCATCATTGCCGCCGGCGTGGCCCACGCCATCACAGCTGCCTGTACCCAGGGCA
ACCTGAGCGACTGTGGCTGCGACAAAGAGAAGCAAGGCCAGTACCACCGGGACGAGGGCTGGAAGTGGGGTGG
CTGCTCCGCCGACATCCGCTACGGCATCGGCTTCGCCAAGGTCTTTGTGGATGCCCGGGAGATCAAGCAGAAT
GCCCGGACTCTCATGAACTTGCACAACAACGAGGCAGGCCGAAAGATCCTGGAGGAGAACATGAAGCTGGAAT
GTAAGTGCCACGGCGTGTCAGGCTCGTGCACCACCAAGACGTGCTGGACCACACTGCCACAGTTTCGGGAGCT
GGGCTACGTACTCAAGGACAAGTACAACGAGGCCGTTCACGTGGAGCCTGTGCGTGCCAGCCGCAACAAGCGG
CCCACCTTCCTGAAGATCAAGAAGCCACTGTCGTACCGCAAGCCCATGGACACGGACCTGGTGTACATCGAGA
AGTCGCCCAACTACTGCGAGGAGGACCCGGTGACCGGCAGTGTGGGCACCCAGGGCCGCGCCTGCAACAAGAC
GGCTCCCCAGGCCAGCGGCTGTGACCTCATGTGCTGTGGGCGTGGCTACAACACCCACCAGTACGCCCGCGTG
TGGCAGTGCAACTGTAAGTTCCACTGGTGCTGCTATGTCAAGTGCAACACGTGCAGCGAGCGCACGGAGATGT
ACACGTGCAAGCTCGAG

| | | | |
|---|---|---|---|
| NOV5d, 228753459 | | | |
| Protein Sequence | SEQ ID NO: 40 | 322 aa | MW at 36054.9kD |

RSLGATVICNKIPGLAPRQRAICQSRPDAIIVIGEGSQMGLDECQFQFRNGRWNCSALGERTVFGKELKVGSR
EAAFTYAIIAAGVAHAITAACTQGNLSDCGCDKEKQGQYHRDEGWKWGGCSADIRYGIGFAKVFVDAREIKQN
ARTLMNLHNNEAGRKILEENMKLECKCHGVSGSCTTKTCWTTLPQFRELGYVLKDKYNEAVHVEPVRASRNKR
PTFLKIKKPLSYRKPMDTDLVYIEKSPNYCEEDPVTGSVGTQGRACNKTAPQASGCDLMCCGRGYNTHQYARV
WQCNCKFHWCCYVKCNTCSERTEMYTCKLE

| | | |
|---|---|---|
| NOV5e, 228753462 | SEQ ID NO: 41 | 966 bp |
| DNA Sequence | ORF Start: at 1 | ORF Stop: end of sequence |

AGATCTCTGGGCGCAACGGTCATCTGTAACAAGATCCCAGGCCTGGCTCCCAGACAGCGGGCGATCTGCCAGA
GCCGGCCCGACGCCATCATCGTCATAGGAGAAGGCTCACAAATGGGCCTCGACGAGTGTCAGTTTCAGTTCCG
CAATGGCCGCTGGAACTGCTCTGCACTGGGAGAGCGCACCGTCTTCGGGAAGGAGCTCAAAGTGGGGAGCCGG
GAGGCTGCATTCACCTACGCCATCATTGCCGCCGGCGTGGTCCACGCCATCACAGCTGCCTGTACCCAGGGCA
ACCTGAGCGACTGTGGCTGCGACAAAGAGAAGCAAGGCCAGTACCACCGGGACGAGGGCTGGAAGTGGGGTGG
CTGCTCCGCCGACATCCGCTACGGCATCGGCTTCGCCAAGGTCTTTGTGGATGCCCGGGAGATCAAGCAGAAT
GCCCGGACTCTCATGAACTTGCACAACAACGAGGCAGGCCGAAAGATCCTGGAGGAGAACATGAAGCTGGAAT
GTAAGTGCCACGGCGTGTCAGGCTCGTGCACCACCAAGACGTGCTGGACCACACTGCCACAGTTTCGGGAGCT
GGGCTACGTGCTCAAGGACAAGTACAACGAGGCCGTTCACGTGGAGCCTGTGCGTGCCAGCCGCAACAAGCGG
CCCACCTTCCTGAAGATCAAGAAGCCACTGTCGTACCGCAAGCCCATGGACACGGACCTGGTGTACATCGAGA
AGTCGCCCAACTACTGCGAGGAGGACCCGGTGACCGGCAGTGTGGGCACCCAGGGCCGCGCCTGCAACAAGAC
GGCTCCCCAGGCCAGCGGCTGTGACCTCATGTGCTGTGGGCGTGGCTACAACACCCACCAGTACGCCCGCGTG
TGGCAGTGCAACTGTAAGTTCCACTGGTGCTGCTATGTCAAGTGCAACACGTGCAGCGAGCGCACGGAGATGT
ACACGTGCAAGCTCGAG

| | | | |
|---|---|---|---|
| NOV5e, 22873462 | | | |
| Protein Sequence | SEQ ID NO: 42 | 322 aa | MW at 36083.0kD |

RSLGATVICNKIPGLAPRQRAICQSRPDAIIVIGEGSQMGLDECQFQFRNGRWNCSALGERTVFGKELKVGSR
EAAFTYAIIAAGVVHAITAACTQGNLSDCGCDKEKQGQYHRDEGWKWGGCSADIRYGIGFAKVFVDAREIKQN
ARTLMNLHNNEAGRKILEENMKLECKCHGVSGSCTTKTCWTTLPQFRELGYVLKDKYNEAVHVEPVRASRNKR
PTFLKIKKPLSYRKPMDTDLVYIEKSPNYCEEDPVTGSVGTQGRACNKTAPQASGCDLMCCGRGYNTHQYARV
WQCNCKFHWCCYVKCNTCSERTEMYTCKLE

| | | |
|---|---|---|
| NoV5f, 228753446 | SEQ ID NO: 43 | 985 bp |
| DNA Sequence | ORF Start: at 2 | ORF Stop: end of sequence |

ATCTGCAGAATTCGCCCTTAGATCTCTGGGCGCAACGGTCATCTGTAACAAGATCCCAGGCCTGGCTCCCAGA
CAGCGGGCGATCTGCCAGAGCCGGCCCGACGCCATCATCGTCATAGGAGAAGGCTCACAAATGGGCCTGGACG
AGTGTCAGTTTCAGTTCCGCAATGGCCGCTGGAACTGCTCTGCACTGGGAGAGCGCACCGTCTTCGGGAAGGA
GCTCAAAGTGGGGAGCCGGGAGGCTGCGTTCACCTACGCCATCATTGCCGCCGGCGTGGCCCACGCCATCACA
GCTGCCTGTACCCAGGGCAACCTCAGCGACTGTGGCTGCGACAAAGAGAAGCAAGGCCAGTACCACCGGGACG
AGGGCTGGAAGTGGGGTGGCTGCTCTGCCGACATCCGCTACGGCATCGGCTTCGCCAAGGTCTTTGTGGATGC
CCGGGAGATCAAGCAGAATGCCCGGACTCTCATGAACTTGCACAACAACGAGGCAGGCCGAAAGATCCTGGAG
GAGAACATGAAGCTGGAATGTAAGTGCCACGGCGTGTCAGGCTCGTGCACCACCAAGACGTGCTGGACCACAC
TGCCACAGTTTCGGGAGCTGGGCTACGTGCTCAAGGACAAGTACAACGAGGCCGTTCACGTGGAGCCTGTGCG
TGCCAGCCGCAACAAGCGGCCCACCTTCCTGAAGATCAAGAAGCCACTGTCGTACCGCAAGCCCATGGACACG
GACCTGGTGTACATCGAGAAGTCGCCCAACTACTGCGAGGAGGACCCGGTGACCGGCAGTGTGGGCACCCAGG
GCCGCGCCTGCAACAAGACGGCTCCCCAGGCCAGCGGCTGTGACCTCATGTGCTGTGGGCGTGGCTACAACAC
CCACCAGTACGCCCGCGTGTGGCAGTGCAACTGTAAGTTCCACTGGTGCTACTATGTCAAGTGCAACACGTGC
AGCGAGCGCACGGAGATGTACACGTGCAAGCTCGAG

| | | | |
|---|---|---|---|
| NOV5f, 228753446 | | | |
| Protein Sequence | SEQ ID NO: 44 | 328 aa | MW at 36733.61kD |

SAEFALRSLGATVICNKIPGLAPRQRAICQSRPDAIIVIGEGSQMGLDECQFQFRNGRWNCSALGERTVFGKE
LKVGSREAAFTYAIIAAGVAHAITAACTQGNLSDCGCDKEKQGQYHRDEGWKWGGCSADIRYGIGFAKVFVDA
REIKQNARTLMNLHNNEAGRKILEENMKLECKCHGVSGSCTTKTCWTTLPQFRELGYVLKDKYNEAVHVEPVR
ASRNKRPTFLKIKKPLSYRKPMDTDLVYIEKSPNYCEEDPVTGSVGTQGRACNKTAPQASGCDLMCCGRGYNT
HQYARVWQCNCKFHWCYYVKCNTCSERTEMYTCKLE

| | | |
|---|---|---|
| NOV5g, 228753465 | SEQ ID NO: 45 | 966 bp |
| DNA Sequence | ORF Start: at 1 | ORF Stop: end of sequence |

AGATCTCTGGGCGCAACGGTCATCTGTAACAAGATCCCAGGCCTGGCTCCCAGACAGCGGGCGATCTGCCAGA
GCCGGCCCGACGCCATCATCGTCATAGGAGAAGGCTCACAAATGGGCCTGGACGAGTGTCAGTTTCAGTTCCG
CAATGGCCGCTGGAACTGCTCTGCACTGGGAGAGCGCACCGTCTTCGGGAAGGAGCTCAAAGTGGGGAGCCGG
GAGGCTGCGTTCACCTACGCCATCATTGCCGCCGGCGTGGCCCACGCCATCACAGCTGCCTGTACCCAGGGCA
ACCTGAGCGACTGTGACTGCGACAAAGAGAAGCAAGGCCAGTACCACCGGGACGAGGGCTGGAAGTGGGGTGG
CTGCTCTGCCGACATCCGCTACGGCATCGGCTTCGCCAAGGTCTTTGTGGATGCCCGGGAGATCAAGCAGAAT
GCCCGGACTCTCATGAACTTGCACAACAACGAGGCAGGCCGAAAGATCCTGGAGGAGAACATGAAGCTGGAAT
GTAAGTGCCACGGCGTGTCAGGCTCGTGCACCACCAAGACGTGCTGGACCACACTGCCACAGTTTCGGGAGCT
GGGCTACGTGCTCAAGGACAAGTACAACGAGGCCGTTCACGTGGAGCCTGTGCGTGCCAGCCGCAACAAGCGG
CCCACCTTCCTGAAGATCAAGAACCCACTGTCGTACCGCAAGCCCATGGACACGGACCTGGTGTACATCGAGA
AGTCGCCCAACTACTGCGAGGAGGACCCGGTGACCGGCAGTGTGGGCACCCAGGGCCGCGCCTGCAACAAGAC
GGCTCCCCAGGCCAGCGGCTGTGACCTCATGTGCTGTGGGCGTGGCTACAACACCCACCAGTACGCCCGCGTG
TGGCAGTACAACTGTAAGTTCCACTGGTGCTGCTATGTCAAGTGCAACACGTGCAGCGAGCGCACGGAGATGT
ACACGTGCAAGCTCGAG

| | | | |
|---|---|---|---|
| NOV5g, 228753465 | | | |
| Protein Sequence | SEQ ID NO: 46 | 322 aa | MW at 36173.0kD |

RSLGATVICNKIPGLAPRQRAICQSRPDAIIVIGEGSQMGLDECQFQFRNGRWNCSALGERTVFGKELKVGSR
EAAFTYAIIAAGVAHAITAACTQGNLSDCDCDKEKQGQYHRDEGWKWGGCSADIRYGIGFAKVFVDAREIKQN
ARTLMNLHNNEAGRKILEENMKLECKCHGVSGSCTTKTCWTTLPQFRELGYVLKDKYNEAVHVEPVRASRNKR
PTFLKIKKPLSYRKPMDTDLVYIEKSPNYCEEDPVTGSVGTQGRACNKTAPQASGCDLMCCGRGYNTHQYARV
WQYNCKFHWCCYVKCNTCSERTEMYTCKLE

| | | |
|---|---|---|
| NOV5h, 228753438 | SEQ ID NO: 47 | 966 bp |
| DNA Sequence | ORF Start: at 1 | ORF Stop: end of sequence |

AGATCTCTGGGCGCAACGGTCATCTGTAACAAGATCCCAGGCCTGGCTCCCAGACAGCGGGCGATCTGCCAGA
GCCGGCCCGACGCCATCATCGTCATAGGAGAAGGCTCACAAATGGGCCTGGACGAGTGTCAGTTTCAGTTCCG
CAATGGCCGCTGGAACTGCTCTGCACTGGGAGAGCGCACCGTCTTCGGGAAGGAGCTCAAAGTGGGGAGCCGG
GAGGCTGCGTTCACCTACGCCATCATTGCCGCCGGCGTGGCCCACGCCATCACAGCTGCCTGTACCCAGGGCA
ACCTGAGCGACTGTGGCTGCGACAAAGAGAAGCAAGGCCAGTACCACCGGGACGAGGGCTGGAAGTGGGGTGG
CTGCTCTGCCGACATCCGCTACGGCATCGGCTTCGCCAAGGTCTTTGTGGATGCCCGGGAGATCAAGCAGAAT
GCCCGGACTCTCATGAACTTGCACAACAACGAGGCAGGCCGAAAGATCCTGGAGGAGAACATGAAGCTGGAAT
GTAAGTGCCACGGCGTGTCAGGCTCGTGCACCACCAAGACGTGCTGGACCACACTGCCACAGTTTCGGGAGCT
GGGCTACGTGCTCAAGGACAAGTACAACGAGGCCGTTCACGTGGAGCCTGTGCGTGCCAGCCGCAACAAGCGG
CCCACCTTCCTGAAGATCAAGAAGCCACTGTCGTACCGCAAGCCCATGGACACGGACCTGGTGTACATCGAGA
AGTCGACCAACTGCTGCGAGGAGGACCCGGTGACCGGCAGTGTGGGCACCCAGGGCCGCGCCTGCAACAAGAC
GGCTCCCCAGGCCAGCGGCTGTGACCTCATGTGCTGTGGGCGTGGCTACAACACCCACCAGTACGCCCGCGTG
TGGCAGTGCAACTGTAAGTTCCACTGGTGCTGCTATGTCAAGTGCAACACGTGCAGCGAGCGCACGGAGATGT
ACACGTGCAAGCTCGAG

| | | | |
|---|---|---|---|
| NOV5h, 228753438 | | | |
| Protein Sequence | SEQ ID NO: 48 | 322 aa | MW at 35998.9kD |

RSLGATVICNKIPGLAPRQRAICQSRPDAIIVIGEGSQMGLDECQFQFRNGRWNCSALGERTVFGKELKVGSR
EAAFTYAIIAAGVAHAITAACTQGNLSDCGCDKEKQGQYHRDEGWKWGGCSADIRYGIGFAKVFVDAREIKQN
ARTLMNLHNNEAGRKILEENMKLECKCHGVSGSCTTKTCWTTLPQFRELGYVLKDKYNEAVHVEPVRASRNKR
PTFLKIKKPLSYRKPMDTDLVYIEKSTNCCEEDPVTGSVGTQGRACNKTAPQASGCDLMCCGRGYNTHQYARV
WQCNCKFHWCCYVKCNTCSERTEMYTCKLE

| | | |
|---|---|---|
| NOV5i, 228753449 | SEQ ID NO: 49 | 966 bp |
| DNA Sequence | ORF Start: at 1 | ORF Stop: end of sequence |

AGATCTCTGGGCGCAACGGTCATCTGTAACAAGATCCCAGGCCTGGCTCCCAGACAGCGGGCGATCTGCCAGA
GCCGGCCCGACGCCATCATCGTCATAGGAGAAGGCTCACAAATGGGCCTGGACGAGTGTCAGTTTCAGTTCCG
CAATGGCCGCTGGAACTGCTCTGCACTGGGAGAGCGCACCGTCTTCGGGAAGGAGCTCAAAGTGGGGAGCCGG
GAGGCTGCGTTCACCTACGCCATCATTGCCGCCGGCGTGGCCCACGCCATCACAGCTGCCTGTACCCAGGGCA
ACCTGAGCGACTGTGGCTGCGACAAAGAGAAGCAAGGCCAGTACCACCGGGACGAGGGCTGGAAGTGGGGTGG
CTGCTCTGCCGACATCCGCTACGGCATCGGCTTCGCCAAGGTCTTTGTGGATGCCCGGGAGATCAAGCAGAAT
GCCCGGACTCTCATGAACTTGCACAACAACGAGGCAGGCCGAAAGATCCTGGAGGAGAACATGAAGCTGGGAT
GTAAGTGCCACGGCGTGTCAGGCTCGTGCACCACCAAGACGTGCTGGACCACACTGCCACAGTTTCGGGAGCT
GGGCTACGTGCTCAAGGACAAGTACAACGAGGCCGTTCACGTGGAGCCTGTGCGTGCCAGCCGCAACAAGCGG
CCCACCTTCCTGAAGATCAAGAAGCCACTGTCGTACCGCAAGCCCATGGACACGGACCTGGTGTACATCGAGA
AGTCGACCAACTGCTGCGAGGAGGACCCGGTGACCGGCAGTCTGGGCACCCAGGGCCGCGCCTGCAACAAGAC
GGCTCCCCAGGCCAGCGGCTGTGACCTCATGTGCTGTGGGCGTGGCTACAACACCCACCAGTACGCCCGCGTG
TGGCAGTGCAACTGTAAGTTCCACTGGTGCTGCTATGTCAAGTGCAACACGTGCAGCGAGCGCACGGAGATGT
ACACGTGCAAGCTCGAG

| | | | |
|---|---|---|---|
| NOV5i, 228753449 | | | |
| Protein Sequence | SEQ ID NO: 50 | 322 aa | MW at 35926.8kD |

RSLGATVICNKIPGLAPRQRAICQSRPDAIIVIGEGSQMGLDECQFQFRNGRWNCSALGERTVFGKELKVGSR
EAAFTYAIIAAGVAHAITAACTQGNLSDCGCDKEKQGQYHRDEGWKWGGCSADIRYGIGFAKVFVDAREIKQN
ARTLMNLNNNEAGRKILEENMKLGCKCHGVSGSCTTKTCWTTLPQFRELGYVLKDKYNEAVHVEPVRASRNKR
PTFLKIKKPLSYRKPMDTDLVYIEKSTNCCEEDPVTCSVGTQGRACNKTAPQASGCDLMCCGRGYNTHQYARV
WQCNCKFHWCCYVKCNTCSERTEMYTCKLE

| | | |
|---|---|---|
| NOV5j, CG50353-02 | SEQ ID NO: 51 | 966 bp |
| DNA Sequence | ORF Start: at 7 | ORF Stop: at 961 |

AGATCTCTGGGCGCAACGGTCATCTGTAACAAGATCCCAGGCCTGGCTCCCAGACAGCGGGCGATCTGCCAGA
GCCGGCCCGACGCCATCATCGTCATAGGAGAAGGCTCACAAATGGGCCTGGACGAGTGTCAGTTTCAGTTCCG
CAATGGCCGCTGGAACTGCTCTGCACTGGGAGAGCGCACCGTCTTCGGGAAGGAGCTCAAAGTGGGGAGCCGG
GAGGCTGCGTTCACCTACGCCATCATTGCCGCCGGCGTGGCCCACGCCATCACAGCTGCCTGTACCCAGGGCA
ACCTGAGCGACTGTGGCTGCGACAAAGAGAAGCAAGGCCAGTACCACCGGGACGAGGGCTGGAAGTGGGGTGG
CTGCTCTGCCGACATCCGCTACGGCATCGGCTTCGCCAAGGTCTTTGTGGATGCCCGGGAGATCAAGCAGAAT
GCCCGGACTCTCATGAACTTGCACAACAACGAGGCAGGCCGAAAGATCCTGGAGGAGAACATGAAGCTGGAAT
GTAAGTGCCACGGCGTGTCAGGCTCGTGCACCACCAAGACGTGCTGGACCACACTGCCACAGTTTCGGGAGCT
GGGCTACGTGCTCAAGGACAAGTACAACGAGGCCGTTCACGTGGAGCCTGTGCGTGCCAGCCGCAACAAGCGG
CCCACCTTCCTGAAGATCAAGAAGCCACTGTCGTACCGCAAGCCCATGGACACGGACCTGGTGTACATCGAGA
AGTCGCCCAACTACTGCGAGGAGGACCCGGTGACCGGCAGTGTGGGCACCCAGGGCCGCGCCTGCAACAAGAC
GGCTCCCCAGGCCAGCGGCTGTGACCTCATGTGCTGTGGGCGTGGCTACAACACCCACCAGTACGCCCGCGTG
TGGCAGTGCAACTGTAAGTTCCACTGGTGCTGCTATGTCAAGTGCAACACGTGCAGCGAGCGCACGGAGATGT
ACACGTGCAAGCTCGAG

| | | | |
|---|---|---|---|
| NOV5j, CG50353-02 | | | |
| Protein Sequence | SEQ ID NO: 52 | 318 aa | MW at 35569.4kD |

LGATVICNKIPGLAPRQRAICQSRPDAIIVIGEGSQMGLDECQFQFRNGRWNCSALGERTVFGKELKVGSREA
AFTYAIIAAGVAHAITAACTQGNLSDCGCDKEKQGQYHRDEGWKWGGCSADIRYGIGFAKVFVDAREIKQNAR
TLMNLHNNEAGRKILEENMKLECKCHGVSGSCTTKTCWTTLPQFRELGYVLKDKYNEAVHVEPVRASRNKRPT
FLKIKKPLSYRKPMDTDLVYIEKSPNYCEEDPVTGSVGTQGRACNKTAPQASGCDLMCCGRGYNTHQYARVWQ
CNCKFHWCCYVKCNTCSERTEMYTCK

| | | |
|---|---|---|
| NOV5k, CG50353-03 | SEQ ID NO: 53 | 1057 bp |
| DNA Sequence | ORF Start: ATG at 1 | ORF Stop: TGA at 1048 |

ATGAACCGGAAAGCGCGGCGCTGCCTGGGCCACCTCTTTCTCAGCCTGGGCATGGTCTGTCTCCTAGCATGTG
GCTTCTCCTCAGTGGTAGCTCTGGGCGCAACGGTCATCTGTAACAAGATCCCAGGCCTGGCTCCCAGACAGCG
GGCGATCTGCCAGAGCCGGCCCGACGCCATCATCGTCATAGGAGAAGGCTCACAAATGGGCCTGGACGAGTGT
CAGTTTCAGTTCCGCAATGGCCGCTGGAACTGCTCTGCACTGGGAGAGCGCACCGTCTTCGGGAAGGAGCTCA
AAGTGGGGAGCCGGGACGGTGCGTTCACCTACGCCATCATTGCCGCCGGCGTGGCCCACGCCATCACAGCTGC
CTGTACCCATGGCAACCTGAGCGACTGTGGCTGCGACAAAGAGAAGCAAGGCCAGTACCACCGGGACGAGGGC
TGGAAGTGGGGTGGCTGCTCTGCCGACATCCGCTACGGCATCGGCTTCGCCAAGGTCTTCGTGGACGCTCGGG
AGATCATGAACAACGCGCGGCGCCTCATGAACCTGCATAACAATGAGGCCGGCAGGAAGGTTCTAGAGGACCG
GATGCAGCTGGAGTGCAAGTGCCACGGCGTGTCTGGCTCCTGCACCACCAAAACCTGCTGGACCACGCTGCCC
AAGTTCCGAGAGGTGGGCCACCTGCTGAAGGAGAAGTACAACGCGGCCGTGCAGGTGGAGGTGGTGCGGGCCA
GCCGTCTGCGGCAGCCCACCTTCCTGCGCATCAAACAGCTGCGCAGCTATCGCAAGCCCATGAAGACGGACCT
GGTGTACATCGAGAAGTCGCCCAACTACTGCGAGCAGGACCCGGTGACCGGCAGTGTGGGCACGCAGGGCCGC
GCCTGCAACAAGACGGCTCCCCAGGCCAGCGGCTGTGACCTCATGTGCTGTGGGCGTGGCTACAACACCCACC
AGTACGCCCGCGTGTGGCAGTGCAACTGTAAGTTCCACTGGTGCTGCTATGTCAAGTGCAACACGTGCAGCGA
GCGCACGGAGATGTACACGTGCAAGTGAGCCCCGT

| | | | |
|---|---|---|---|
| NOV5k, CG50353-03 | | | |
| Protein Sequence | SEQ ID NO: 54 | 349 aa | MW at 38980.7kD |

MNRKARRCLGHLFLSLGMVCLLACGFSSVVALGATVICNKIPGLAPRQRAICQSRPDAIIVIGEGSQMGLDEC
QFQFRNGRWNCSALGERTVFGKELKVGSRDGAFTYAIIAAGVAHAITAACTHGNLSDCGCDKEKQGQYHRDEG
WKWGGCSADIRYGIGFAKVFVDAREIMKNARRLMNLHNNEAGRKVLEDRMQLECKCHGVSGSCTTKTCWTTLP
KFREVGHLLKEKYNAAVQVEVVRASRLRQPTFLRIKQLRSYRKPMKTDLVYIEKSPNYCEEDPVTGSVGTQGR
ACNKTAPQASGCDLMCCGRGYNTHQYARVWQCNCKFHWCCYVKCNTCSERTEMYTCK

| | | |
|---|---|---|
| | SEQ ID NO: 55 | 1628 bp |
| NOV5l, SNP13382474 of | ORF Start: ATG at 1 | ORF Stop: TGA at 1048 |
| CG50353-01, DNA Sequence | SNP Pos: 951 | SNP Change: G to T |

ATGAACCGGAAAGCGCGGCGCTGCCTGGGCCACCTCTTTCTCAGCCTGGGCATGGTCTGTCTCCTAGCATGTG
GCTTCTCCTCAGTGGTAGCTCTGGGCGCAACGGTCATCTGTAACAAGATCCCAGGCCTGGCTCCCAGACAGCG
GGCGATCTGCCAGAGCCGGCCCGACGCCATCATCGTCATAGGAGAAGGCTCACAAATGGGCCTGGACGAGTGT
CAGTTTCAGTTCCGCAATGGCCGCTGGAACTGCTCTGCACTGGGAGAGCGCACCGTCTTCGGGAAGGAGCTCA
AAGTGGGGAGCCGGGACGGTGCGTTCACCTACGCCATCATTGCCGCCGGCGTGGCCCACGCCATCACAGCTGC
CTGTACCCATGGCAACCTGAGCGACTGTGGCTGCGACAAAGAGAAGCAAGGCCAGTACCACCGGGACGAGGGC
TGGAAGTGGGGTGGCTGCTCTGCCGACATCCGCTACGGCATCGGCTTCGCCAAGGTCTTCGTGGACGCTCGGG
AGATCATGAAGAACGCGCGGCGCCTCATGAACCTGCATAACAATGAGGCCGGCAGGAAGGTTCTAGAGGACCG
GATGCAGCTGGAGTGCAAGTGCCACGGCGTGTCTGGCTCCTGCACCACCAAAACCTGCTGGACCACGCTGCCC
AAGTTCCGAGAGGTGGGCCACCTGCTGAAGGAGAAGTACAACGCGGCCGTGCAGGTGGAGGTGGTGCGGGCCA
GCCGTCTGCGGCAGCCCACCTTCCTGCGCATCAAACAGCTGCGCAGCTATCGCAAGCCCATGAAGACGGACCT
GGTGTACATCGAGAAGTCGCCCAACTACTGCGAGGAGGACCCGGTGACCGGCAGTGTGGGCACGCAGGGCCGC
GCCTGCAACAAGACGGCTCCCCAGGCCAGCGGCTGTGACCTCATGTGCTGTGGGCGTGGCTACAACACCCACC
ATTACGCCCGCGTGTGGCAGTGCAACTGTAAGTTCCACTGGTGCTGCTATGTCAAGTGCAACACGTGCAGCGA
GCGCACGGAGATGTACACGTGCAAGTGAGCCCCGTGTGCACACCACCCTCCCGCTGCAAGTCAGATTGCTGGG
AGGACTGGACCGTTTCCAAGCTGCGGGCTCCCTGGCAGGATGCTGAGCTTGTCTTTTCTGCTGACGAGGGTAC
TTTTCCTGGGTTTCCTGCAGGCATCCGTGGGGGAAAAAAAATCTCTCAGAGCCCTCAACTATTCTGTTCCACA
CCCAATGCTGCTCCACCCTCCCCCAGACACAGCCCAGGTCCCTCCGCGGCTGGAGCGAAGCCTTCTGCAGCAG
GAACTCTGGACCCCTGGGCCTCATCACAGCAATATTTAACAATTTATTCTGATAAAAATAATATTAATTTATT
TAATTAAAAAGAATTCTTCCACCTCGTCGGGATCCGTTTTCTGCAATCAAAGTGGACTGCTTGCTTTCCTAGC
AGGATGATTTTGTTGCTAGGACAAGGAGCCGTGTAGAAGTGTACATAACTATTCTTTATGCAGATATTTCTAC
TAGCTGATTTTGCAGGTACCCACCTTGCAGCACTAGATGTTTAAGTACAAGAGGAGACATCTTTTATGCATAT
ATAGATATACACACACGAAAAA

| | | | |
|---|---|---|---|
| NOV5l, SNP13382474 of | SEQ ID NO: 56 | | MW at 38989.7kD |
| CG50353-01, Protein Sequence | SNP Pos: 317 | 349 aa | SNP Change: Gln to His |

MNRKARRCLGHLFLSLGMVCLLACGFSSVVALGATVICNKIPGLAPRQRAICQSRPDAIIVIGEGSQMGLDEC
QFQFRNGRWNCSALGERTVFGKELKVGSRDGAFTYAIIAAGVAHAITAACTHGNLSDCGCDKEKQGQYHRDEG
WKWGGCSADIRYGIGFAKVFVDAREIMKNARRLMNLHNNEAGRKVLEDRMQLECKCHGVSGSCTTKTCWTTLP
KFREVGHLLKEKYNAAVQVEVVRASRLRQPTFLRIKQLRSYRKPMKTDLVYIEKSPNYCEEDPVTGSVGTQGR
ACNKTAPQASGCDLMCCGRGYNTHHYARVWQCNCKFHWCCYVKCNTCSERTEMYTCK

A ClustalW comparison of the above protein sequences yields the following sequence alignment shown in Table 5B.

TABLE 5B
Comparison of the NOV5 protein sequences.

| | |
|---|---|
| NOV5a | MNRKARRCLGHLFLSLGMVCLLACGFSSVVALGATVICNKIPGLAPRQRAICQSRPDAII |
| NOV5b | -----------------------------RSLGATVICNKIPGLAPRQRAICQSRPDAII |
| NOV5c | -----------------------------RSLGATVICNKIPGLAPRQRAICQSRPDAII |
| NOV5d | -----------------------------RSLGATVICNKIPGLAPRQRAICQSRPDAII |
| NOV5e | -----------------------------RSLGATVICNKIPGLAPRQRAICQSRPDAII |
| NOV5f | -----------------------SAEFALRSLGATVICNKIPGLAPRQRAICQSRPDAII |
| NOV5g | -----------------------------RSLGATVICNKIPGLAPRQRAICQSRPDAII |
| NOV5h | -----------------------------RSLGATVICNKIPGLAPRQRAICQSRPDAII |
| NOV5i | -----------------------------RSLGATVICNKIPGLAPRQRAICQSRPDAII |
| NOV5j | -------------------------------LGATVICNKIPGLAPRQRAICQSRPDAII |
| NOV5k | MNRKARRCLGHLFLSLGMVCLLACGFSSVVALGATVICNKIPGLAPRQRAICQSRPDAII |
| NOV5a | VIGEGSQMGLDECQFQFRNGRWNCSALGERTVFGKELKVGSRDGAFTYAIIAAGVAHAIT |
| NOV5b | VIGEGSQMGLDECQFQFRNGRWNCSALGERTVFGKELKVGSREAAFTYAIIAAGVAHAIT |
| NOV5c | VIGEGSQMGLDECQFQFRNGRWNCSALGERTVFGKELKVGSREAAFTYAIIAAGVAHAIT |
| NOV5d | VIGEGSQMGLDECQFQFRNGRWNCSALGERTVFGKELKVGSREAAFTYAIIAAGVAHAIT |
| NOV5e | VIGEGSQMGLDECQFQFRNGRWNCSALGERTVFGKELKVGSREAAFTYAIIAAGVVHAIT |
| NOV5f | VIGEGSQMGLDECQFQFRNGRWNCSALGERTVFGKELKVGSREAAFTYAIIAAGVAHAIT |
| NOV5g | VIGEGSQMGLDECQFQFRNGRWNCSALGERTVFGKELKVGSREAAFTYAIIAAGVAHAIT |
| NOV5h | VIGEGSQMGLDECQFQFRNGRWNCSALGERTVFGKELKVGSREAAFTYAIIAAGVAHAIT |
| NOV5i | VIGEGSQMGLDECQFQFRNGRWNCSALGERTVFGKELKVGSREAAFTYAIIAAGVAHAIT |
| NOV5j | VIGEGSQMGLDECQFQFRNGRWNCSALGERTVFGKELKVGSREAAFTYAIIAAGVAHAIT |
| NOV5k | VIGEGSQMGLDECQFQFRNGRWNCSALGERTVFGKELKVGSRDGAFTYAIIAAGVAHAIT |
| NOV5a | AACTHGNLSDCGCDKEKQGQYHRDEGWKWGGCSADIRYGIGFAKVFVDAREIMKNARRLM |
| NOV5b | AACTQGNLSDCGCDKEKQGQYHRDEGWKWGGCSADIRYGIGFAKVFVDAREIKQNARTLM |
| NOV5c | AACTQGNLSDCGCDKEKQGQYHRDEGWKWGGCSADIRYGIGFAKVFVDAREIKQNARTLM |
| NOV5d | AACTQGNLSDCGCDKEKQGQYHRDEGWKWGGCSADIRYGIGFAKVFVDAREIKQNARTLM |
| NOV5e | AACTQGNLSDCGCDKEKQGQYHRDEGWKWGGCSADIRYGIGFAKVFVDAREIKQNARTLM |
| NOV5f | AACTQGNLSDCGCDKEKQGQYHRDEGWKWGGCSADIRYGIGFAKVFVDAREIKQNARTLM |
| NOV5g | AACTQGNLSDCDCDKEKQGQYHRDEGWKWGGCSADIRYGIGFAKVFVDAREIKQNARTLM |
| NOV5h | AACTQGNLSDCGCDKEKQGQYHRDEGWKWGGCSADIRYGIGFAKVFVDAREIKQNARTLM |
| NOV5i | AACTQGNLSDCGCDKEKQGQYHRDEGWKWGGCSADIRYGIGFAKVFVDAREIKQNARTLM |
| NOV5j | AACTQGNLSDCGCDKEKQGQYHRDEGWKWGGCSADIRYGIGFAKVFVDAREIKQNARTLM |
| NOV5k | AACTHGNLSDCGCDKEKQGQYHRDEGWKWGGCSADIRYGIGFAKVFVDAREIMKNARRLM |
| NOV5a | NLHNNEAGRKVLEDRMQLECKCHGVSGSCTTKTCWTTLPKFREVGHLLKEKYNAAVQVEV |
| NOV5b | NLHNNEAGRKILEENMKLECKCHGVSGSCTTKTCWTTLPQFRELGYVLKDKYNEAVHVEP |
| NOV5c | NLHNNEAGRKILEENMKLECKCHGVSGSCTTKTCWTTLPQFRELGYVLKDKYNEAVHVEP |
| NOV5d | NLHNNEAGRKILEENMKLECKCHGVSGSCTTKTCWTTLPQFRELGYVLKDKYNEAVHVEP |
| NOV5e | NLHNNEAGRKILEENMKLECKCHGVSGSCTTKTCWTTLPQFRELGYVLKDKYNEAVHVEP |
| NOV5f | NLHNNEAGRKILEENMKLECKCHGVSGSCTTKTCWTTLPQFRELGYVLKDKYNEAVHVEP |
| NOV5g | NLHNNEAGRKILEENMKLECKCHGVSCSCTTKTCWTTLPQFRELGYVLKDKYNEAVHVEP |
| NOV5h | NLHNNEAGRKILEENMKLECKCHGVSGSCTTKTCWTTLPQFRELGYVLKDKYNEAVHVEP |
| NOV5i | NLHNNEAGRKILEENMKLGCKCHGVSGSCTTKTCWTTLPQFRELGYVLKDKYNEAVHVEP |
| NOV5j | NLHNNEAGRKILEENMKLECKCHGVSGSCTTKTCWTTLPQFRELGYVLKDKYNEAVHVEP |
| NOV5k | NLHNNEAGRKVLEDRMQLECKCHGVSGSCTTKTCWTTLPKFREVGHLLKEKYNAAVQVEV |
| NOV5a | VRASRLRQPTFLRIKQLRSYRKPMKTDLVYIEKSPNYCEEDPVTGSVGTQGRACNKTAPQ |
| NOV5b | VRASRNKRPTFLKIKKPLSYRKPMDTDLVYIEKSPNYCEEDPVTGSVGTQGRACNKTAPQ |
| NOV5c | VRASRNKRPTFLKIKKPLSYRKPMDTDLVYIEKSPNYCEEDPVTGSVGTQGRACNKTAPQ |
| NOV5d | VRASRNKRPTFLKIKKPLSYRKPMDTDLVYIEKSPNYCEEDPVTGSVGTQGRACNKTAPQ |
| NOV5e | VRASRNKRPTFLKIKKPLSYRKPMDTDLVYIEKSPNYCEEDPVTGSVGTQGRACNKTAPQ |
| NOV5f | VRASRNKRPTFLKIKKPLSYRKPMDTDLVYIEKSPNYCEEDPVTGSVGTQGRACNKTAPQ |
| NOV5g | VRASRNKRPTFLKIKKPLSYRKPMDTDLVYIEKSPNYCEEDPVTGSVGTQGRACNKTAPQ |
| NOV5h | VRASRNKRPTFLKIKKPLSYRKPMDTDLVYIEKSTNCCEEDPVTGSVGTQGRACNKTAPQ |
| NOV5i | VRASRNKRPTFLKIKKPLSYRKPMDTDLVYIEKSTNCCEEDPVTGSVGTQGRACNKTAPQ |
| NOV5j | VRASRNKRPTFLKIKKPLSYRKPMDTDLVYIEKSPNYCEEDPVTGSVCTQGRACNKTAPQ |
| NOV5k | VRASRLRQPTFLRIKQLRSYRKPMKTDLVYIEKSPNYCEEDPVTGSVGTQGRACNKTAPQ |
| NOV5a | ASGCDLMCCGRGYNTHQYARVWQCNCKFHWCCYVKCNTCSERTEMYTCK-- |
| NOV5b | ASGCDLMCCGRGYNTHQYARVWQCNCKFHWCCYVKCNTCSERTEMYTCKLE |
| NOV5c | ASGCDLMCCGRGYNTHQYARVWQCNCKFHWCCYVKCNTCSERTEMYTCKLE |
| NOV5d | ASGCDLMCCGRGYNTHQYARVWQCNCKFHWCVYVKCNTCSERTEMYTCKLE |
| NOV5e | ASGCDLMCCGRGYNTHQYARVWQCNCKFHWCCYVKCNTCSERTEMYTCKLE |
| NOV5f | ASGCDLMCCGRGYNTHQYARVWQCNCKFHWCYYVKCNTCSERTEMYTCKLE |
| NOV5g | ASGCDLMCCGRGYNTHQYARVWQYNCKFHWCCYVKCNTCSERTEMYTCKLE |
| NOV5h | ASGCDLMCCGRGYNTHQYARVWQCNCKFHWCCYVKCNTCSERTEMYTCKLE |
| NOV5i | ASGCDLMCCGRGYNTHQYARVWQCNCKFHWCCYVKCNTCSERTEMYTCKLE |
| NOV5j | ASGCDLMCCGRGYNTHQYARVWQCNCKFHWCCYVKCNTCSERTEMYTCK-- |
| NOV5k | ASGCDLMCCGRGYNTHQYARVWQCNCKFHWCCYVKCNTCSERTEMYTCK-- |
| NOV5a | (SEQ ID NO: 34) |
| NOV5b | (SEQ ID NO: 36) |
| NOV5c | (SEQ ID NO: 38) |
| NOV5d | (SEQ ID NO: 40) |
| NOV5e | (SEQ ID NO: 42) |
| NOV5f | (SEQ ID NO: 44) |
| NOV5g | (SEQ ID NO: 46) |
| NOV5h | (SEQ ID NO: 48) |
| NOV5i | (SEQ ID NO: 50) |
| NOV5j | (SEQ ID NO: 52) |
| NOV5k | (SEQ ID NO: 54) |

Further analysis of the NOV5a protein yielded the following properties shown in Table 5C.

TABLE 5C
Protein Sequence Properties NOV5a

| SignalP | Cleavage site between residues 32 and 33 | | | |
|---|---|---|---|---|
| analysis: | | | | |
| PSORT II | PSG: a new signal peptide prediction method | | | |
| analysis: | N-region: length 7; pos. chg 4; neg. chg 0 | | | |
| | H-region: length 32; peak value 10.30 | | | |
| | PSG score: 5.90 | | | |
| | GvH: von Heijne's method for signal seq. recognition | | | |
| | GvH score (threshold: −2.1): −0.60 | | | |
| | possible cleavage site: between 27 and 28 | | | |
| | >>> Seems to have a cleavable signal peptide (1 to 27) | | | |
| | ALOM: Klein et al's method for TM region allocation | | | |
| | Init position for calculation: 28 | | | |
| | Tentative number of TMS(s) for the threshold 0.5: 2 | | | |
| | Number of TMS(s) for threshold 0.5: 0 | | | |
| | PERIPHERAL | Likelihood = 6.89 (at 151) | | |
| | ALOM score: 0.05 (number of TMSs: 0) | | | |
| | MTOP: Prediction of membrane topology (Hartmann et al.) | | | |
| | Center position for calculation: 13 | | | |
| | Charge difference: −2.5 C(3.0)-N(5.5) | | | |
| | N >= C: N-terminal side will be inside | | | |
| | MITDISC: discrimination of mitochondrial targeting seq | | | |
| | R content: | 6 | Hyd Moment(75): | 11.39 |
| | Hyd Moment(95): | 16.83 | G content: | 5 |
| | D/E content: | 1 | S/T content: | 5 |

```
Score: 1.59
Gavel: prediction of cleavage sites for mitochondrial preseq
R-2 motif at 65 SRP|DA
NUCDISC: discrimination of nuclear localization signals
pat4: none
pat7: none
bipartite: none
content of basic residues: 14.6%
NLS Score: −0.47
KDEL: ER retention motif in the C-terminus: none
ER Membrane Retention Signals:
XXRR-like motif in the N-terminus: NRKA
none
SKL: peroxisomal targeting signal in the C-terminus: none
PTS2: 2nd peroxisomal targeting signal: none
VAC: possible vacuolar targeting motif: found
TLPK at 217
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 55.5
COIL: Lupas's algorithm to detect coiled-coil regions
total: 0 residues
Final Results (k = 9/23):
65.2%: mitochondrial
13.0%: Golgi
  8.7%: extracellular, including cell wall
  8.7%: endoplasmic reticulum
  4.3%: cytoplasmic
>> prediction for CG50353-01 is mit (k = 23)
```

A search of the NOV5a protein against the Geneseq database, a proprietary database that contains sequences published in patents and patent publication, yielded several homologous proteins shown in Table 5D.

TABLE 5D
Geneseq Results for NOV5a

| | | NOV5a | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| ABJ10594 | Human novel protein NOV5a SEQ ID | 1 . . . 349 | 349/349 (100%) | 0.0 |
| | NO: 16 - Homo sapiens, 349 aa. | 1 . . . 349 | 349/349 (100%) | |
| | [WO200259315-A2, 01 AUG. 2002] | | | |
| AAY57598 | Human Wnt-7a protein - Homo | 1 . . . 349 | 321/349 (91%) | 0.0 |
| | sapiens, 349 aa. [WO9957248-A1, | 1 . . . 349 | 335/349 (95%) | |
| | 11 NOV. 1999] | | | |
| AAY70737 | Human Wnt-7a protein - Homo | 1 . . . 349 | 321/349 (91%) | 0.0 |
| | sapiens, 349 aa. [WO200021555-A1, | 1 . . . 349 | 335/349 (95%) | |
| | 20 APR. 2000] | | | |
| AAB19789 | Human Wnt-7a protein involved in | 1 . . . 349 | 321/349 (91%) | 0.0 |
| | kidney tubulogenesis - Homo sapiens, | 1 . . . 349 | 335/349 (95%) | |
| | 349 aa. [WO200061630-A1, | | | |
| | 19 OCT. 2000] | | | |
| AAE34043 | WNT-7A protein - Unidentified, 349 | 1 . . . 349 | 317/349 (90%) | 0.0 |
| | aa. [WO200290992-A2, | 1 . . . 349 | 333/349 (94%) | |
| | 14 NOV. 2002] | | | |

In a BLAST search of public sequence databases, the NOV5a protein was found to have homology to the proteins shown in the BLASTP data in Table 5E.

TABLE 5E
Public BLASTP Results for NOV5a

| | | NOV5a | Identities/ | |
|---|---|---|---|---|
| Protein | | Residues/ | Similarities for | |
| Accession | | Match | the Matched | Expect |
| Number | Protein/Organism/Length | Residues | Portion | Value |
| O00755 | Wnt-7a protein precursor - Homo | 1 . . . 349 | 321/349 (91%) | 0.0 |
| | sapiens (Human), 349 aa. | 1 . . . 349 | 335/349 (95%) | |
| Q96H90 | Hypothetical protein - Homo sapiens | 1 . . . 349 | 317/349 (90%) | 0.0 |
| | (Human), 349 aa. | 1 . . . 349 | 333/349 (94%) | |
| AAH49093 | Hypothetical protein - Mus | 1 . . . 349 | 315/349 (90%) | 0.0 |
| | musculus (Mouse), 433 aa | 85 . . . 433 | 332/349 (94%) | |
| | (fragment). | | | |
| Q9DBY3 | Wingless-related MMTV integration | 1 . . . 349 | 315/349 (90%) | 0.0 |
| | site 7A - Mus musculus (Mouse), | 1 . . . 349 | 332/349 (94%) | |
| | 349 aa. | | | |
| P24383 | Wnt-7a protein precursor - Mus | 1 . . . 349 | 313/349 (89%) | 0.0 |
| | musculus (Mouse), 349 aa. | 1 . . . 349 | 330/349 (93%) | |

PFam analysis predicts that the NOV5a protein contains the domains shown in the Table 5F.

TABLE 5F
Domain Analysis of NOV5a

| | | Identities/ | |
|---|---|---|---|
| Pfam | | Similarities | Expect |
| Domain | NOV5a Match Region | for the Matched Region | Value |
| wnt | 37 . . . 349 | 180/352 (51%) | 3.2e−212 |
| | | 298/352 (85%) | |

### Example 6

The NOV6 clone was analyzed, and the nucleotide and encoded polypeptide sequences are shown in Table 6A.

TABLE 6A
NOV6 Sequence Analysis

| | | |
|---|---|---|
| NOV6a, CG50709-03 | SEQ ID NO: 57 | 993 bp |
| DNA Sequence | ORF Start: at 1 | ORF Stop: end of sequence |

CTGACCGGGCGGGAAGTCCTGACGCCCTTCCCAGGATTGGGCACTGCGGCAGCCCCGGCACAGGGCGGGGCCC
ACCTGAAGCAGTGTGACCTGCTGAAGCTGTCCCGGCGGCAGAAGCAGCTCTGCCGGAGGGAGCCCGGCCTGGC
TGAGACCCTGAGGGATGCTGCGCACCTCGGCCTGCTTGAGTGCCAGTTTCAGTTCCGGCATGAGCGCTGGAAC
TGTAGCCTGGAGGGCAGGACGGGCCTGCTCAAGAGAGGCTTCAAAGAGACAGCTTTCCTGTACGCGGTGTCCT
CTGCCGCCCTCACCCACACCCTGGCCCGGGCCTGCAGCGCTGGGCGCATGGAGCGCTGCACCTGTGATGACTC
TCCGGGGCTGGAGAGCCGACAGGCCTGGCAATGGGGCGTGTGCGGTGACAACCTCAAGTACAGCACCAAGTTT
CTGAGCAACTTCCTGGGGTCCAAGAGAGCAAACAAGGACCTGCGGGCACGGGCAGACGCCCACAATACCCACG
TGGGCATCAAGGCTGTGAAGAGTGGCCTCAGGACCACGTGTAAGTGCCATGGCGTATCAGGCTCCTGTGCCGT
GCGCACCTGCTGGAAGCAGCTCTCCCCGTTCCGTGAGACGGGCCAGGTGCTGAAACTGCGCTATGACTCGGCT
GTCAAGGTGTCCAGTGCCACCAATGAGGCCTTGGGCCGCCTAGAGCTGTGGGCCCCTGCCAGGCAGGGCAGCC
TCACCAAAGGCCTGGCCCCAAGGTCTGGGGACCTGGTGTACATGGAGGACTCACCCAGCTTCTGCCGGCCCAG
CAAGTACTCACCTGGCACAGCAGGTAGGGTGTGCTCCCGGGAGGCCAGCTGCAGCAGCCTGTGCTGCGGGCGG
GGCTATGACACCCAGAGCCGCCTGGTGGCCTTCTCCTGCCACTGCCAGGTGCAGTGGTGCTGCTACGTGGAGT
GCCAGCAATGTGTGCAGGAGGAGCTTGTGTACACCTGCAAGCAC

| | | | |
|---|---|---|---|
| NOV6a, CG50709-03 | | | |
| Protein Sequence | SEQ ID NO: 58 | 331 aa | MW at 36432.2kD |

LTGREVLTPFPGLGTAAPAQGGAHLKQCDLLKLSRRQKQLCRREPGLAETLRDAAHLGLLECQFQFRHERWN
CSLEGRTGLLKRGFKETAFLYAVSSAALTHTLARACSAGRMERCTCDDSPGLESRQAWQWGVCGDNLKYSTKF
LSNFLGSKRGNKDLRARADAHNTHVGIKAVKSGLRTTCKCHGVSGSCAVRTCWKQLSPFRETGQVLKLRYDSA
VKVSSATNEALGRLELWAPARQGSLTKGLAPRSGDLVYMEDSPSFCRPSKYSPGTAGRVCSREASCSSLCCGR
GYDTQSRLVAFSCHCQVQWCCYVECQQCVQEELVYTCKH

| | | |
|---|---|---|
| NOV6b, 282997951 | SEQ ID NO: 59 | 928 bp |
| DNA Sequence | ORF Start: at 2 | ORF Stop: end of sequence |

CACCGGATCCCAGTGTGACCTGCTGAAGCTGTCCCGGCGGCAGAAGCAGCTCTGCCGGAGGGAGCCCGGCCTG
GCTGAGACCCTGAGGGATGCTGCGCACCTCGGCCTGCTTGAGTGCCAGTTTCAGTTCCGGCATGAGCGCTGGA
ACTGTAGCCTGGAGGGCAGGACGGGCCTGCTCAAGAGAGGCTTCAAAGAGACAGCTTTCCTGTACGCGGTGTC
CTCTGCCGCCCTCACCCACACCCTGGCCCGGGCCTGCAGCGCTGGGCGCATGGAGCGCTGCACCTGTGATGAC
TCTCCGGGGCTGGAGAGCCGGCAGGCCTGGCAGTGGGGCGTGTGCGGTGACAACCTCAAGTACAGCACCAAGT
TTCTGAGCAACTTCCTGGGGTCCAAGAGAGCAAACAAGGACCTGCGGGCACGGGCAGACGCCCACAATACCCA
CGTGGGCATCAAGGCTGTGAAGAGTGGCCTCAGGACCACGTGTAAGTGCCATGGCGTATCAGGCTCCTGTGCC
GTGCGCACCTGCTGGAAGCAGCTCTCCCCGTTCCGTGAGACGGGCCAGGTGCTGAAACTGCGCTATGACTCGG
CTGTCAAGGTGTCCAGTGCCACCAATGAGGCCTTGGGCCGCCTAGAGCTGTGGGCCCCTGCCAGGCAGGGCAG
CCTCACCAAAGGCCTGGCCCCAAGGTCTGGGGACCTGGTGTACATGGAGGACTCACCCAGCTTCTGCCGGCCC
AGCAAGTACTCACCTGGCACAGCAGGTAGGGTGTGCTCCCGGGAGGCCAGCTGCAGCAGCCTGTGCTGCGGGC
GGGGCTATGACACCCAGAGCCGCCTGGTGGCCTTCTCCTGCCACTGCCAGGTGCAGTGGTGCTGCTACGTGGA
GTGCCAGCAATGTGTGCAGGAGGAGCTTGTGTACACCTGCAAGCTCGAGGGC

| | | | |
|---|---|---|---|
| NOV6b, 282997951 | | | |
| Protein Sequence | SEQ ID NO: 60 | 309 aa | MW at 34226.6kD |

TGSQCDLLKLSRRQKQLCRREPGLAETLRDAAHLGLLECQFQFRHERWNCSLEGRTGLLKRGFKETAFLYAVS
SAALTHTLARACSAGRMERCTCDDSPGLESRQAWQWGVCGDNLKYSTKFLSNFLGSKRGNKDLRARADAHNTH
VGIKAVKSGLRTTCKCHGVSGSCAVRTCWKQLSPFRETGQVLKLRYDSAVKVSSATNEALGRLELWAPARQGS
LTKGLAPRSGDLVYMEDSPSFCRPSKYSPGTAGRVCSREASCSSLCCGRGYDTQSRLVAFSCHCQVQWCCYVE
CQQCVQEELVYTCKLEG

| | | |
|---|---|---|
| NOV6c, CG50709-05 | SEQ ID NO: 61 | 1464 bp |
| DNA Sequence | ORF Start: ATG at 38 | ORF Stop: TAG at 1109 |

GCGAGGAGATGCTAGAGGGCGCAGCGCCGCCAGCACCATGCGCCCCCCGCCCGCGCTGGCCCTGGCCGGGCTC
TGCCTGCTGGCGCTGCCCGCCGCCGCCGCCTCCTACTTCGGCCTGACCGGGCGGGAAGTCCTGACGCCCTTCC
CAGGATTGGGCACTGCGGCAGCCCCGGCACAGGGCGGGGCCCACCTGAAGCAGTGTGACCTGCTGAAGCTGTC
CCGGCGGCAGAAGCAGCTCTGCCGGAGGGAGCCCGGCCTGGCTGAGACCCTGAGGGATGCTGCGCACCTCGGC
CTGCTTGAGTGCCAGTTTCAGTTCCGGCATGAGCGCTGGAACTGTAGCCTGGAGGGCAGGACGGGCCTGCTCA
AGAGAGGCTTCAAAGAGACAGCTTTCCTGTACGCGGTGTCCTCTGCCGCCCTCACCCACACCCTGGCCCGGGC
CTGCAGCGCTGGGCGCATGGAGCGCTGCACCTGTGATGACTCTCCGGGGCTGGAGAGCCGGCAGGCCTGGCAG
TGGGGCGTGTGCGGTGACAACCTCAAGTACAGCACCAAGTTTCTGAGCAACTTCCTGGGGTCCAAGAGAGGAA
ACAAGGACCTGCGGGCACGGGCAGACGCCCACAATACCCACGTGGGCATCAAGGCTGTGAAGAGTGGCCTCAG
GACCACGTGTAAGTGCCATGGCGTATCAGGCTCCTGTGCCGTGCGCACCTGCTGGAAGCAGCTCTCCCCGTTC
CGTGAGACGGGCCAGGTGCTGAAACTGCGCTATGACTCGGCTGTCAAGGTGTCCAGTGCCACCAATGAGGCCT
TGGGCCGCCTAGAGCTGTGGGCCCCTGCCAGGCAGGGCAGCCTCACCAAAGGCCTGGCCCCAAGGTCTGGGGA
CCTGGTGTACATGGAGGACTCACCCAGCTTCTGCCGGCCCAGCAAGTACTCACCTGGCACAGCAGGTAGGGTG
TGCTCCCGGGAGGCCAGCTGCAGCAGCCTGTGCTGCGGGCGGGGCTATGACACCCAGAGCCGCCTGGTGGCCT
TCTCCTGCCACTGCCAGGTGCAGTGGTGCTGCTACGTGGAGTGCCAGCAATGTGTGCAGGAGGAGCTTGTGTA
CACCTGCAAGCACTAGGCCTACTGCCCAGCAAGCCAGTCTGGCACTGCCAGGACCTCCTGTGGCACCCTTCAA
GCTGCCCAGCCGGCCCTCTGGGCAGACTGTCATCACATGCATGCATAAACCGGCATGTGTGCCAATGCACACG
AGTGTGCCACTCACCACCATTCCTTGGCCAGCCTTTTGCCTCCCTCGATACTCAACAAAGAGAAGCAAAGCCT
CCTCCCTTAACCCAAGCATCCCCAACCTTGTTGAGGACTTGGAGAGGAGGGCAGAGTGAGAAAGACATGGAGG
GAAATAAGGGAGACCAAGAGCACAGCAGGACTGAAATTTTGGACGGGAGAGAGGGGCTATTCCATCTTGCTTC
CTGG

| | | | |
|---|---|---|---|
| NOV6c, CG50709-05 | | | |
| Protein Sequence | SEQ ID NO: 62 | 357 aa | MW at 38970.2kD |

65MRPPPALALAGLCLLALPAAAASYFGLTGREVLTPFPGLGTAAAPAQGGAHLKQCDLLKLSRRQKQLCRREPG
LAETLRDAAHLGLLECQFQFRHERWNCSLEGRTGLLKRGFKETAFLYAVSSAALTHTLARACSAGRMERCTCD
DSPGLESRQAWQWGVCGDNLKYSTKFLSNFLGSKRGNKDLRARADAHNTHVGIKAVKSGLRTTCKCHGVSGSC
AVRTCWKQLSPFRETGQVLKLRYDSAVKVSSATNEALGRLELWAPARQGSLTKGLAPRSGDLVYMEDSPSFCR
PSKYSPGTAGRVCSREASCSSLCCGRGYDTQSRLVAFSCHCQVQWCCYVECQQCVQEELVYTCKH

| | | |
|---|---|---|
| NOV6d, 277582109 | SEQ ID NO: 63 | 1093 bp |
| DNA Sequence | ORF Start: at 2 | ORF Stop: end of sequence |

CACCGGATCCACCATGCGCCCCCCGCCCGCGCTGGCCCTGGCCGGGCTCTGCCTGCTGGCGCTGCCCGCCGCC
GCCGCCTCCTACTTCGGCCTGACCGGGCGGGAAGTCCTGACGCCCTTCCCAGGATTGGGCACTGCGGCAGCCC
CGGCACAGGGCGGGGCCCACCTGAAGCAGTGTGACCTGCTGAAGCTGTCCCGGCGGCAGAAGCAGCTCTGCCG
GAGGGAGCCCGGCCTGGCTGAGACCCTGAGGGATGCTGCGCACCTCGGCCTGCTTGAGTGCCAGTTTCAGTTC
CGGCATGAGCGCTGGAACTGTAGCCTGGAGGGCAGGACGGGCCTGCTCAAGAGAGGCTTCAAAGAGACAGCTT
TCCTGTACGCGGTGTCCTCTGCCGCCCTCACCCACACCCTGGCCCGGGCCTGCAGCGCTCGGCGCATGGAGCG
CTGCACCTGTGATGACTCTCCGGGGCTGGAGAGCCGGCAGGCCTGGCAGTGGGGCGTGTGCGGTGACAACCTC
AAGTACAGCACCAAGTTTCTGAGCAACTTCCTGGGGTCCAAGAGAGGAAACAAGGACCTGCGGGCACGGGCAG
ACGCCCACAATACCCACGTGGGCATCAAGGCTGTGAAGAGTGGCCTCAGGACCACGTGTAAGTGCCATGGCGT
ATCAGGCTCCTGTGCCGTGCGCACCTGCTGGAAGCAGCTCTCCCCGTTCCGTGAGACGGGCCAGGTGCTGAAA
CTGCGCTATGACTCGGCTGTCAAGGTGTCCAGTGCCACCAATGAGGCCTTGGGCCGCCTAGAGCTGTGGGCCC
CTGCCAGGCAGGGCAGCCTCACCAAAGGCCTGGCCCCAAGGTCTGGGGACCTGGTGTACATGGAGGACTCACC
CAGCTTCTGCCGGCCCAGCAAGTACTCACCTGGCACAGCAGGTAGGGTGTGCTCCCGGGAGGCCAGCTGCAGC
AGCCTGTGCTGCGGGCGGGGCTATGACACCCAGAGCCGCCTGGTGGCCTTCTCCTGCCACTGCCAGGTGCAGT
GGTGCTGCTACGTGGAGTGCCAGCAATGTGTGCAGGAGGAGCTTGTGTACACCTGCAAGCACCTCGAGGGC

| | | | |
|---|---|---|---|
| NOV6d, 277582109 | | | |
| Protein Sequence | SEQ ID NO: 64 | 364 aa | MW at 39615.9kD |

TGSTMRPPPALALAGLCLLALPAAAASYFGLTGREVLTPFPGLGTAAAPAQGGAHLKQCDLLKLSRRQKQLCR
REPGLAETLRDAAHLGLLECQFQFRHERWNCSLEGRTGLLKRGFKETAFLYAVSSAALTHTLARACSAGRMER
CTCDDSPGLESRQAWQWGVCGDNLKYSTKFLSNFLGSKRGNKDLRARADAHNTHVGIKAVKSGLRTTCKCHGV
SGSCAVRTCWKQLSPFRETGQVLKLRYDSAVKVSSATNEALGRLELWAPARQGSLTKGLAPRSGDLVYMEDSP
SFCRPSKYSPGTAGRVCSREASCSSLCCGRGYDTQSRLVAFSCHCQVQWCCYVECQQCVQEELVYTCKHLEG

| | | |
|---|---|---|
| NOV6e, 277582117 | SEQ ID NO: 65 | 1024 bp |
| DNA Sequence | ORF Start: at 2 | ORF Stop: end of sequence |

AACCGGATCCTCCTACTTCGGCCTGACCGGGCGGGAAGTCCTGACGCCCTTCCCAGGATTGGGCACTGCGGCA
GCCCCGGCACAGGGCGGGGCCCACCTGAAGCAGTGTGACCTGCTGAAGCTGTCCCGGCGGCAGAAGCAGCTCT
GCCGGAGGGAGCCCGGCCTGGCTGAGACCCTGAGGGATGCTGCGCACCTCGGCCTGCTTGAGTGCCAGTTTCA
GTTCCGGCATGAGCGCTGGAACTGTAGCCTGGAGGGCAGGACGGGCCTGCTCAAGAGAGGCTTCAAAGAGACA
GCTTTCCTGTACGCGGTGTCCTCTGCCGCCCTCACCCACACCCTGGCCCGGGCCTGCAGCGCTGGGCGCATGG
AGCGCTGCACCTGTGATGACTCTCCGGGGCTCGAGAGCCGGCAGGCCTGGCAGTGGGGCGTGTGCGGTGACAA
CCTCAAGTACAGCACCAAGTTTCTGAGCAACTTCCTGGGGTCCAAGAGAGGAAACAAGGACCTGCGGGCACGG
GCAGACGCCCACAATACCCACGTGGGCATCAAGGCTGTGAAGAGTGGCCTCAGGACCACGTGTAAGTGCCATC
GCGTATCAGGCTCCTGTGCCGTGCGCACCTGCTGGAAGCAGCTCTCCCCGTTCCGTGAGACGGGCCAGGTGCT
GAAACTGCGCTATGACTCGGCTGTCAAGGTGTCCAGTGCCACCAATGAGGCCTTGGGCCGCCTAGAGCTGTGG
GCCCCTGCCAGGCAGGGCAGCCTCACCAAAGGCCTGGCCCCAAGGTCTGGGGACCTGGTGTACATGGAGGACT
CACCCAGCTTCTGCCGGCCCAGCAAGTACTCACCTGGCACAGCAGGTAGGGTGTGCTCCCGGGAGGCCAGCTG
CAGCAGCCTGTGCTGCGGGCGGGGCTATGACACCCAGAGCCGCCTGGTGGCCTTCTCCTGCCACTGCCAGGTG
CAGTGGTGCTGCTACGTGGAGTGCCAGCAATGTGTGCAGGAGGAGCTTGTGTACACCTGCAAGCACCTCGAGG
GC

| | | | |
|---|---|---|---|
| NOV6e, 277582117 | | | |
| Protein Sequence | SEQ ID NO: 66 | 341 aa | MW at 37431.2kD |

TGSSYFGLTGREVLTPFPGLGTAAAPAQGGAHLKQCDLLKLSRRQKQLCRREPGLAETLRDAAHLGLLECQFQ
FRHERWNCSLEGRTGLLKRGFKETAFLYAVSSAALTHTLARACSAGRMERCTCDDSPGLESRQAWQWGVCGDN
LKYSTKFLSNFLGSKRGNKDLRARADAHNTHVGIKAVKSGLRTTCKCHGVSCSCAVRTCWKQLSPFRETGQVL
KLRYDSAVKVSSATNEALGRLELWAPARQGSLTKGLAPRSGDLVYMEDSPSFCRPSKYSPGTAGRVCSREASC
SSLCCGRGYDTQSRLVAFSCHCQVQWCCYVECQQCVQEELVYTCKHLEG

| | | |
|---|---|---|
| NOV6f, CG50709-01 | SEQ ID NO: 67 | 1021 bp |
| DNA Sequence | ORF Start: at 3 | ORF Stop: TAG at 996 |

TCCTGACCGGGCGGGAAGTCCTGACGCCCTTCCCAGGATTGGGCACTGCGGCAGCCCCGGCACAGGGCGGGGC
CCACCTGAAGCAGTGTGACCTGCTGAAGCTGTCCCGGCGGCAGAAGCAGCTCTGCCGGAGGGAGCCCGGCCTG
GCTGAGACCCTGAGGGATGCTGCGCACCTCCGCCTGCTTGAGTGCCAGTTTCAGTTCCGGCATGAGCGCTGGA
ACTGTAGCCTGGAGGGCAGGATGGGCCTGCTCAAGAGAGGCTTCAAAGAGACAGCTTTCCTGTACGCGGTGTC
CTCTGCCGCCCTCACCCACACCCTGGCCCGGGCCTGCAGCGCTGGGCGCATGGAGCGCTGCACCTGTGATGAC
TCTCCGGGGCTGGAGAGCCGGCAGGCCTGGCAGTGGGGCGTGTGCGGTGACAACCTCAACTACAGCACCAAGT
TTCTGAGCAACTTCCTGGGGTCCAAGAGAGGAAACAAGGACCTGCGGGCACGGGCAGACGCCCACAATACCCA
CGTGGGCATCAAGGCTGTGAAGAGTGGCCTCAGGACCACGTGTAAGTGCCATGGCGTATCAGGCTCCTGTGCC
GTGCGCACCTGCTGGAAGCAGCTCTCCCCGTTCCGTGAGACGGGCCAGGTGCTCAAACTGCGCTATGACTCGG
CTGTCAAGGTGTCCAGTGCCACCAATGAGGCCTTGGGCCGCCTAGAGCTGTGGGCCCCTGCCAGGCAGGGCAG
CCTCACCAAAGGCCTGGCCCCAAGGTCTGGGGACCTGGTGTACATGGAGGACTCACCCAGCTTCTGCCGGCCC
AGCAAGTACTCACCTGGCACAGCAGGTAGGGTGTGCTCCCGGGAGGCCAGCTGCAGCAGCCTGTGCTGCGGGC
GGGGCTATGACACCCAGAGCCGCCTGGTGGCCTTCTCCTGCCACTGCCAGGTGCAGTGGTGCTGCTACGTGGA
GTGCCAGCAATGTGTGCAGGAGGAGCTTGTGTACACCTGCAAGCACTAGGCCTACTGCCCAGCAAGCCAGTC

| | | | |
|---|---|---|---|
| NOV6f, CG50709-01 | | | |
| Protein Sequence | SEQ ID NO: 68 | 331 aa | MW at 36462.3kD |

LTGREVLTPFPGLGTAAAPAQGGAHLKQCDLLKLSRRQKQLCRREPGLAETLRDAAHLGLLECQFQFRHERWN
CSLEGRMGLLKRGFKETAFLYAVSSAALTHTLARACSAGRMERCTCDDSPGLESRQAWQWGVCGDNLKYSTKF
LSNFLGSKRGNKDLRARADAHNTHVGIKAVKSGLRTTCKCHGVSGSCAVRTCWKQLSPFRETGQVLKLRYDSA
VKVSSATNEALGRLELWAPARQGSLTKGLAPRSGDLVYMEDSPSFCRPSKYSPGTAGRVCSREASCSSLCCGR
GYDTQSRLVAFSCHCQVQWCCYVECQQCVQEELVYTCKH

| | | |
|---|---|---|
| NOV6g, CG50709-02 | SEQ ID NO: 69 | 933 bp |
| DNA Sequence | ORF Start: ATG at 274 | ORF Stop: TAG at 928 |

GGGGCCCACCTGAAGCAGTGTGACCTGCTGAAGCTGTCCCGGCGGCAGAAGCAGCTCTGCCGGAGGGAGCCCG
GCCTGGCTGAGACCCTGAGGGATGCTGCGCACCTCGGCCTGCTTGAGTGCCAGTTTCAGTTCCGGCATGAGCG
CTGGAACTGTAGCCTGGAGGGCAGGACGGGCCTGCTCAAGAGAGGCTTCAAAGAGACAGCTTTCCTGTACGCG
GTGTCCTCTGCCGCCCTCACCCACACCCTGGCCCGGGCCTGCAGCGCTGGGCGCATGGAGCGCTGCACCTGTG
ATGACTCTCCGGGGCTGGAGAGCCGGCAGGCCTGGCAGTGGGGCGTGTGCGGTGACAACCTCAAGTACAGCAC
CAAGTTTCTGAGCAACTTCCTGGGGTCCAAGAGAGGAAACAAGGACCTGCGGGCACGGGCAGACGCCCACAAT
ACCCACGTGGGCATCAAGGCTGTGAAGAGTGGCCTCAGGACCACGTGTAAGTGCCATGGCGTATCAGGCTCCT
GTGCCGTGCGCACCTGCTGGAAGCAGCTCTCCCCGTTCCGTGAGACGGGCCAGGTGCTGAAACTGCGCTATGA
CTCGGCTGTCAAGGTGTCCAGTGCCACCAATGAGGCCTTGGGCCGCCTAGAGCTGTGGGCCCCTGCCAGGCAG
GGCAGCCTCACCAAAGGCCTGGCCCCAAGGTCTGGGGACCTGGTGTACATGGAGGACTCACCCAGCTTCTGCC
GGCCCAGCAAGTACTCACCTGGCACAGCAGGTAGGGTGTGCTCCCGGGAGGCCAGCTGCAGCAGCCTGTGCTG
CGGGCGGGGCTATGACACCCAGAGCCGCCTGGTGGCCTTCTCCTGCCACTGCCAGGTGCAGTGGTGCTGCTAC
GTGGAGTGCCAGCAATGTGTGCAGGAGGAGCTGGTGTACACCTGCAAGCACTAGGCC

| | | | |
|---|---|---|---|
| NOV6g, CG50709-02 | | | |
| Protein Sequence | SEQ ID NO: 70 | 218 aa | MW at 24076.1kD |

MERCTCDDSPGLESRQAWQWGVCGDNLKYSTKFLSNFLGSKRGNKDLRARADAHNTHVGIKAVKSGLRTTCKC
HGVSGSCAVRTCWKQLSPFRETGQVLKLRYDSAVKVSSATNEALGRLELWAPARQGSLTKGLAPRSGDLVYME
DSPSFCRPSKYSPGTAGRVCSREASCSSLCCGRGYDTQSRLVAFSCHCQVQWCCYVECQQCVQEELVYTCKH

| | | |
|---|---|---|
| NOV6h, CG50709-04 | SEQ ID NO: 71 | 849 bp |
| DNA Sequence | ORF Start: at 1 | ORF Stop: end of sequence |

AAGCAGTGTGACCTGCTGAAGCTGTCCCGGCGGCAGAAGCAGCTCTGCCGGAGGGAGCCCGGCCTGGCTGAGA
CCCTGAGGGATGCTGCGCACCTCGGCCTGCTTGAGTGCCAGTTTCAGTTCCGGCATGAGCGCTGGAACTCTAG
CCTGGAGGGCAGGATGGGCCTGCTCAAGAGAGGCTTCAAAGAGACAGCTTTCCTGTACGCGGTGTCCTCTGCC
GCCCTCACCCACACCCTGGCCCGGGCCTGCAGCGCTGGGCGCATGGAGCGCTGCACCTGTGATGACTCTCCGG
GGCTGGAGAGCCGGCAGGCCTGGCAGTGGGGCGTGTGCGGTGACAACCTCAAGTACAGCACCAAGTTTCTGAG
CAACTTCCTGGGGTCCAAGAGAGGAAACAAGGACCTGCGGGCACGGGCAGACGCCCACAATACCCACGTGGGC
ATCAAGGCTGTGAAGAGTGGCCTCAGGACCACGTGTAAGTGCCATGGCGTATCAGGCTCCTGTGCCGTGCGCA
CCTGCTGGAAGCAGCTCTCCCCGTTCCGTGAGACGGGCCAGGTGCTGAAACTGCGCTATGACTCGGCTGTCAA
GGTGTCCAGTGCCACCAATGAGGCCTTGGGCCGCCTAGAGCTGTGGGCCCCTGCCAGGCAGGGCAGCCTCACC
AAAGGCCTGGCCCCAAGGTCTGGGGACCTGGTGTACATGGAGGACTCACCCAGCTTCTGCCGGCCCAGCAAGT
ACTCACCTGGCACAGCAGGTAGGGTGTGCTCCCGGGAGGCCAGCTGCAGCAGCCTGTGCTGCGGGCGGGGCTA
TGACACCCAGAGCCGCCTGGTGGCCTTCTCCTGCCACTGCCAGGTG

| | | | |
|---|---|---|---|
| NOV6h, CG50709-04 | | | |
| Protein Sequence | SEQ ID NO: 72 | 283 aa | MW at 31272.4kD |

KQCDLLKLSRRQKQLCRREPGLAETLRDAAHLGLLECQFQFRHERWNCSLEGRMGLLKRGFKETAFLYAVSSA
ALTHTLARACSAGRMERCTCDDSPGLESRQAWQWGVCGDNLKYSTKFLSNFLGSKRGNKDLRARADAHNTHVG
IKAVKSGLRTTCKCHGVSGSCAVRTCWKQLSPFRETGQVLKLRYDSAVKVSSATNEALGRLELWAPARQGSLT
KGLAPRSGDLVYMEDSPSFCRPSKYSPGTAGRVCSREASCSSLCCGRGYDTQSRLVAFSCHCQV

| | | |
|---|---|---|
| NOV6i, CG50709-06 | SEQ ID NO: 73 | 1093 bp |
| DNA Sequence | ORF Start: ATG at 14 | ORF Stop: end of sequence |

CACCGGATCCACCATGCGCCCCCCGCCCGCGCTGGCCCTGGCCGGGCTCTGCCTGCTGGCGCTGCCCGCCGCC
GCCGCCTCCTACTTCGGCCTGACCGGGCGGGAAGTCCTGACGCCCTTCCCAGGATTGGGCACTGCGGCAGCCC
CGGCACAGGGCGGGGCCCACCTGAAGCAGTGTGACCTGCTGAAGCTGTCCCGGCGGCAGAAGCAGCTCTGCCG
GAGGGAGCCCGGCCTGGCTGAGACCCTGAGGGATGCTGCGCACCTCGGCCTGCTTGAGTGCCAGTTTCAGTTC
CGGCATGAGCGCTGGAACTGTAGCCTGGAGGGCAGGACGGGCCTGCTCAAGAGAGGCTTCAAAGAGACAGCTT
TCCTGTACGCGGTGTCCTCTGCCGCCCTCACCCACACCCTGGCCCGGGCCTGCAGCGCTGGGCGCATGGAGCG
CTGCACCTGTGATGACTCTCCGGGGCTGGAGAGCCGGCAGGCCTGGCAGTGGGGCGTGTGCGGTGACAACCTC
AAGTACAGCACCAAGTTTCTGAGCAACTTCCTGGGGTCCAAGAGAGGAAACAAGGACCTGCGGGCACGGGCAG
ACGCCCACAATACCCACGTGGGCATCAAGGCTGTGAAGAGTGGCCTCAGGACCACGTGTAAGTGCCATGGCGT
ATCAGGCTCCTGTGCCGTGCGCACCTGCTGGAAGCAGCTCTCCCCGTTCCGTGAGACGGGCCAGGTGCTGAAA
CTGCGCTATGACTCGGCTGTCAAGGTGTCCAGTGCCACCAATGAGGCCTTGGGCCGCCTAGAGCTGTGGGCCC
CTGCCAGGCAGGGCAGCCTCACCAAAGGCCTGGCCCCAAGGTCTGGGGACCTGGTGTACATGGAGGACTCACC
CAGCTTCTGCCGGCCCAGCAAGTACTCACCTGGCACAGCAGGTAGGGTGTGCTCCCGGGAGGCCAGCTGCAGC
AGCCTGTGCTGCGGGCGGGGCTATGACACCCAGAGCCGCCTGGTGGCCTTCTCCTGCCACTGCCAGGTGCAGT
GGTGCTGCTACGTGGAGTGCCAGCAATGTGTGCAGGAGGAGCTTGTGTACACCTGCAAGCACCTCGAGGGC

| | | | |
|---|---|---|---|
| NOV6i, CG50709-06 | | | |
| Protein Sequence | SEQ ID NO: 74 | 360 aa | MW at 39269.6kD |

MRPPPALALAGLCLLALPAAAASYFGLTGREVLTPFPGLGTAAAPAQGGAHLKQCDLLKLSRRQKQLCRREPG
LAETLRDAAHLGLLECQFQFRHERWNCSLEGRTGLLKRGFKETAFLYAVSSAALTHTLARACSAGRMERCTCD
DSPGLESRQAWQWGVCGDNLKYSTKFLSNFLGSKRGNKDLRARADAHNTHVGIKAVKSGLRTTCKCHGVSGSC
AVRTCWKQLSPFRETGQVLKLRYDSAVKVSSATNEALGRLELWAPARQGSLTKGLAPRSGDLVYMEDSPSFCR
PSKYSPGTAGRVCSREASCSSLCCGRGYDTQSRLVAFSCHCQVQWCCYVECQQCVQEELVYTCKHLEG

| | | |
|---|---|---|
| NOV6j, CG50709-07 | SEQ ID NO: 75 | 1024 bp |
| DNA Sequence | ORF Start: at 2 | ORF Stop: end of sequence |

AACCGGATCCTCCTACTTCGGCCTGACCGGGCGGGAAGTCCTGACGCCCTTCCCAGGATTGGGCACTGCGGCA
GCCCCGGCACAGGGCGGGGCCCACCTGAAGCAGTGTGACCTGCTGAAGCTGTCCCGGCGGCAGAAGCAGCTCT
GCCGGAGGGAGCCCGGCCTGGCTGAGACCCTGAGGGATGCTGCGCACCTCGGCCTGCTTGAGTGCCAGTTTCA
GTTCCGGCATGAGCGCTGGAACTGTAGCCTGGAGGGCAGGACGGGCCTGCTCAAGAGAGGCTTCAAAGAGACA
GCTTTCCTGTACGCGGTGTCCTCTGCCGCCCTCACCCACACCCTGGCCCGGGCCTGCAGCGCTGGGCGCATGG
AGCGCTGCACCTGTGATGACTCTCCGGGGCTGGAGAGCCGGCAGGCCTGGCAGTGGGGCGTGTGCGGTGACAA
CCTCAAGTACAGCACCAAGTTTCTGAGCAACTTCCTGGGGTCCAAGAGAGGAAACAAGGACCTGCGGGCACGG
GCAGACGCCCACAATACCCACGTGGGCATCAAGGCTGTGAAGAGTGGCCTCAGGACCACGTGTAAGTGCCATG
GCGTATCAGGCTCCTGTGCCGTGCGCACCTGCTGGAAGCAGCTCTCCCCGTTCCGTGAGACGGGCCAGGTGCT
GAAACTGCGCTATGACTCGGCTGTCAAGGTGTCCAGTGCCACCAATGAGGCCTTGGGCCGCCTAGAGCTGTGG
GCCCCTGCCAGGCAGGGCAGCCTCACCAAAGGCCTGGCCCCAAGGTCTGGGGACCTGGTGTACATGGAGGACT
CACCCAGCTTCTGCCGGCCCAGCAAGTACTCACCTGGCACAGCAGGTAGGGTGTGCTCCCGGGAGGCCAGCTG
CAGCAGCCTGTGCTGCGGGCGGGGCTATGACACCCAGAGCCGCCTGGTGGCCTTCTCCTGCCACTGCCAGGTG
CAGTGGTGCTGCTACGTGGAGTGCCAGCAATGTGTGCAGGAGGAGCTTGTGTACACCTGCAAGCACCTCGAGG
GC

| | | | |
|---|---|---|---|
| NOV6j, CG50709-07 | | | |
| Protein Sequence | SEQ ID NO: 76 | 341 aa | MW at 37431.2kD |

TGSSYFGLTGREVLTPFPGLGTAAAPAQGGAHLKQCDLLKLSRRQKQLCRREPGLAETLRDAAHLGLLECQFQ
FRHERWNCSLEGRTGLLKRGFKETAFLYAVSSAALTHTLARACSAGRMERCTCDDSPGLESRQAWQWGVCGDN
LKYSTKFLSNFLGSKRGNKDLRARADAHNTHVGIKAVKSGLRTTCKCHGVSGSCAVRTCWKQLSPFRETGQVL
KLRYDSAVKVSSATNEALGRLELWAPARQGSLTKGLAPRSGDLVYMEDSPSFCRPSKYSPGTAGRVCSREASC
SSLCCGRGYDTQSRLVAFSCHCQVQWCCYVECQQCVQEELVYTCKHLEG

| | | |
|---|---|---|
| | SEQ ID NO: 77 | 993 bp |
| NOV6k, SNP13381605 of | ORF Start: at 1 | ORF Stop: end of sequence |
| CG50709-03, DNA Sequence | SNP Pos: 653 | SNP Change: C to T |

CTGACCGGGCGGGAAGTCCTGACGCCCTTCCCAGGATTGGGCACTGCGGCAGCCCCGGCACAGGGCGGGGCCC
ACCTGAAGCAGTGTGACCTGCTGAAGCTGTCCCGGCGGCAGAAGCAGCTCTGCCGGAGGGAGCCCGGCCTGGC
TGAGACCCTGAGGGATGCTGCGCACCTCGGCCTGCTTGAGTGCCAGTTTCAGTTCCGGCATGAGCGCTGGAAC
TGTAGCCTGGAGGGCAGGACGGGCCTGCTCAAGAGAGGCTTCAAAGAGACAGCTTTCCTGTACGCGGTGTCCT
CTGCCGCCCTCACCCACACCCTGGCCCGGGCCTGCAGCGCTGGGCGCATGGAGCGCTGCACCTGTGATGACTC
TCCGGGGCTGGAGAGCCGACAGGCCTGGCAATGGGGCGTGTGCGGTGACAACCTCAAGTACAGCACCAAGTTT
CTGAGCAACTTCCTGGGGTCCAAGAGAGGAAACAAGGACCTGCGGGCACGGGCAGACGCCCACAATACCCACG
TGGGCATCAAGGCTGTGAAGAGTGGCCTCAGGACCACGTGTAAGTGCCATGGCGTATCAGGCTCCTGTGCCGT
GCGCACCTGCTGGAAGCAGCTCTCCCCGTTCCGTGAGACGGGCCAGGTGCTGAAACTGCGCTATGACTTGGCT
GTCAAGGTGTCCAGTGCCACCAATGAGGCCTTGGGCCGCCTAGAGCTGTGGGCCCCTGCCAGGCAGGGCAGCC
TCACCAAAGGCCTGGCCCCAAGGTCTGGGGACCTGGTGTACATGGAGGACTCACCCAGCTTCTGCCGGCCCAG
CAAGTACTCACCTGGCACAGCAGGTAGGGTGTGCTCCCGGGAGGCCAGCTGCAGCAGCCTGTGCTGCGGGCGG
GGCTATGACACCCAGAGCCGCCTGGTGGCCTTCTCCTGCCACTGCCAGGTGCAGTGGTGCTGCTACGTGGAGT
GCCAGCAATGTGTGCAGGAGGAGCTTGTGTACACCTGCAAGCAC

| | | | |
|---|---|---|---|
| NOV6k, SNP13381605 of | SEQ ID NO: 78 | | MW at 36458.3kD |
| CG50709-03, Protein Sequence | SNP Pos: 218 | 331 aa | SNP Change: Ser to Leu |

LTGREVLTPFPGLGTAAAPAQGGAHLKQCDLLKLSRRQKQLCRREPGLAETLRDAAHLGLLECQFQFRHERWN
CSLEGRTGLLKRGFKETAFLYAVSSAALTHTLARACSAGRMERCTCDDSPGLESRQAWQWGVCGDNLKYSTKF
LSNFLGSKRGNKDLRARADAHNTHVGIKAVKSGLRTTCKCHGVSGSCAVRTCWKQLSPFRETGQVLKLRYDLA
VKVSSATNEALGRLELWAPARQGSLTKGLAPRSGDLVYMEDSPSFCRPSKYSPGTAGRVCSREASCSSLCCGR
GYDTQSRLVAFSCHCQVQWCCYVECQQCVQEELVYTCKH

| | | |
|---|---|---|
| | SEQ ID NO: 79 | 993 bp |
| NOV6l, SNP13381606 of | ORF Start: at 1 | ORF Stop: end of sequence |
| CG50709-03, DNA Sequence | SNP Pos: 743 | SNP Change: T to C |

CTGACCGGGCGGGAAGTCCTGACGCCCTTCCCAGGATTGGGCACTGCGGCAGCCCCGGCACAGGGCGGGGCCC
ACCTGAAGCAGTGTGACCTGCTGAAGCTGTCCCGGCGGCAGAAGCAGCTCTGCCGGAGGGAGCCCGGCCTGGC
TGAGACCCTGAGGGATGCTGCGCACCTCGGCCTGCTTGAGTGCCAGTTTCAGTTCCGGCATGAGCGCTGGAAC
TGTAGCCTGGAGGGCAGGACGGGCCTGCTCAAGAGAGGCTTCAAAGAGACAGCTTTCCTGTACGCGGTGTCCT
CTGCCGCCCTCACCCACACCCTGGCCCGGGCCTGCAGCGCTGGGCGCATGGAGCGCTGCACCTGTGATGACTC
TCCGGGGCTGGAGAGCCGACAGGCCTGGCAATGGGGCGTGTGCGGTGACAACCTCAAGTACAGCACCAAGTTT
CTGAGCAACTTCCTGGGGTCCAAGAGAGGAAACAAGGACCTGCGGGCACGGGCAGACGCCCACAATACCCACG
TGGGCATCAAGGCTGTGAAGAGTGGCCTCAGGACCACGTGTAAGTGCCATGGCGTATCAGGCTCCTGTGCCGT
GCGCACCTGCTGGAAGCAGCTCTCCCCGTTCCGTGAGACGGGCCAGGTGCTGAAACTGCGCTATGACTCGGCT
GTCAAGGTGTCCAGTGCCACCAATGAGGCCTTGGGCCGCCTAGAGCTGTGGGCCCCTGCCAGGCAGGGCAGCC
TCACCAAAGGCCCGGCCCCAAGGTCTGGGGACCTGGTGTACATGGAGGACTCACCCAGCTTCTGCCGGCCCAG
CAAGTACTCACCTGGCACAGCAGGTAGGGTGTGCTCCCGGGAGGCCAGCTGCAGCAGCCTGTGCTGCGGGCGG
GGCTATGACACCCAGAGCCGCCTGGTGGCCTTCTCCTGCCACTGCCAGGTGCAGTGGTGCTGCTACGTGGAGT
GCCAGCAATGTGTGCAGGAGGAGCTTGTGTACACCTGCAAGCAC

| | | | |
|---|---|---|---|
| NOV6l, SNP13381606 of | SEQ ID NO: 80 | | MW at 36416.2kD |
| CG50709-03, Protein Sequence | SNP Pos: 248 | 331 aa | ISNP Change: Leu to Pro |

LTGREVLTPFPGLGTAAAPAQGGAHLKQCDLLKLSRRQKQLCRREPGLAETLRDAAHLGLLECQFQFRHERWN
CSLEGRTGLLKRGFKETAFLYAVSSAALTHTLARACSAGRMERCTCDDSPGLESRQAWQWGVCGDNLKYSTKF
LSNFLGSKRGNKDLRARADAHNTHVGIKAVKSGLRTTCKCHGVSGSCAVRTCWKQLSPFRETGQVLKLRYDSA
VKVSSATNEALGRLELWAPARQGSLTKGPAPRSGDLVYMEDSPSFCRPSKYSPGTAGRVCSREASCSSLCCGR
GYDTQSRLVAFSCHCQVQWCCYVECQQCVQEELVYTCKH

| | | |
|---|---|---|
| | SEQ ID NO: 81 | 993 bp |
| NOV6m, SNP13378337 of | ORF Start: at 1 | ORF Stop: end of sequence |
| CG50709-03, DNA Sequence | SNP Pos: 764 | SNP Change: T to C |

CTGACCGGGCGGGAAGTCCTGACGCCCTTCCCAGGATTGGGCACTGCGGCAGCCCCGGCACAGGGCGGGGCCC
ACCTGAAGCAGTGTGACCTGCTGAAGCTGTCCCGGCGGCAGAAGCAGCTCTGCCGGAGGGAGCCCGGCCTGGC
TGAGACCCTGAGGGATGCTGCGCACCTCGGCCTGCTTGAGTGCCAGTTTCAGTTCCGGCATGAGCGCTGGAAC
TGTAGCCTGGAGGGCAGGACGGGCCTGCTCAAGAGAGGCTTCAAAGAGACAGCTTTCCTGTACGCGGTGTCCT
CTGCCGCCCTCACCCACACCCTGGCCCGGGCCTGCAGCGCTGGGCGCATGGAGCGCTGCACCTCTGATGACTC
TCCGGGGCTGGAGAGCCGACAGGCCTGGCAATGGGGCGTGTGCGGTGACAACCTCAAGTACAGCACCAAGTTT
CTGAGCAACTTCCTGGGGTCCAAGAGAGGAAACAAGGACCTGCGGGCACGGGCAGACGCCCACAATACCCACG
TGGGCATCAAGGCTGTGAAGAGTGGCCTCAGGACCACGTGTAAGTGCCATGGCGTATCAGGCTCCTGTGCCGT
GCGCACCTGCTGGAAGCAGCTCTCCCCGTTCCGTGAGACGGGCCAGGTGCTGAAACTGCGCTATGACTCGGCT
GTCAAGGTGTCCAGTGCCACCAATGAGGCCTTGGGCCGCCTAGAGCTGTGGGCCCCTGCCAGGCAGGGCAGCC
TCACCAAAGGCCTGGCCCCAAGGTCTGGGGACCCGGTGTACATGGAGGACTCACCCAGCTTCTGCCGGCCCAG
CAAGTACTCACCTGGCACAGCAGGTAGGGTGTGCTCCCGGGAGGCCAGCTGCAGCAGCCTGTGCTGCGGGCGG
GGCTATGACACCCAGAGCCGCCTGGTGGCCTTCTCCTGCCACTGCCAGGTGCAGTGGTGCTGCTACGTGGAGT
GCCAGCAATGTGTGCAGGAGGAGCTTGTGTACACCTGCAAGCAC

| | | | |
|---|---|---|---|
| NOV6m, SNP13378337 of | SEQ ID NO: 82 | | MW at 36416.2kD |
| CG50709-03, Protein Sequence | SNP Pos: 255 | 331 aa | SNP Change: Leu to Pro |

LTGREVLTPFPGLGTAAAPAQGGAHLKQCDLLKLSRRQKQLCRREPGLAETLRDAAHLGLLECQFQFRHERWN
CSLEGRTGLLKRGFKETAFLYAVSSAALTHTLARACSAGRMERCTCDDSPGLESRQAWQWGVCGDNLKYSTKF
LSNFLGSKRGNKDLRARADAHNTHVGIKAVKSGLRTTCKCHGVSGSCAVRTCWKQLSPFRETGQVLKLRYDSA
VKVSSATNEALGRLELWAPARQGSLTKGLAPRSGDPVYMEDSPSFCRPSKYSPGTAGRVCSREASCSSLCCGR
GYDTQSRLVAFSCHCQVQWCCYVECQQCVQEELVYTCKH

| | | |
|---|---|---|
| | SEQ ID NO: 83 | 993 bp |
| NOV6n, SNP13381607 of | ORF Start: at 1 | ORF Stop: end of sequence |
| CG50709-03, DNA Sequence | SNP Pos: 799 | SNP Change: C to T |

CTGACCGGGCGGGAAGTCCTGACGCCCTTCCCAGGATTGGGCACTGCGGCAGCCCCGGCACAGGGCGGGGCCC
ACCTGAAGCAGTGTGACCTGCTGAAGCTGTCCCGGCGGCAGAAGCAGCTCTGCCGGAGGGAGCCCGGCCTGGC
TGAGACCCTGAGGGATGCTGCGCACCTCGGCCTGCTTGAGTGCCAGTTTCAGTTCCGGCATGAGCGCTGGAAC
TGTAGCCTGGAGGGCAGGACGGGCCTGCTCAAGAGAGGCTTCAAAGAGACAGCTTTCCTGTACGCGGTGTCCT
CTGCCGCCCTCACCCACACCCTGGCCCGGGCCTGCAGCGCTGGGCGCATGGAGCGCTGCACCTGTGATGACTC
TCCGGGGCTGGAGAGCCGACAGGCCTGGCAATGGGGCGTGTGCGGTGACAACCTCAAGTACAGCACCAAGTTT
CTGAGCAACTTCCTGGGGTCCAAGAGAGGAAACAAGGACCTGCGGGCACGGGCAGACGCCCACAATACCCACG
TGGGCATCAAGGCTGTGAAGAGTGGCCTCAGGACCACGTGTAAGTGCCATGGCGTATCAGGCTCCTGTGCCGT
GCGCACCTGCTGGAAGCAGCTCTCCCCGTTCCGTGAGACGGGCCAGGTGCTGAAACTGCGCTATGACTCGGCT
GTCAAGGTGTCCAGTGCCACCAATGAGGCCTTGGGCCGCCTAGAGCTGTGGGCCCCTGCCAGGCAGGGCAGCC
TCACCAAAGGCCTGGCCCCAAGGTCTGGGGACCTGGTGTACATGGAGGACTCACCCAGCTTCTGCCGGTCCAG
CAAGTACTCACCTGGCACAGCAGGTAGGGTGTGCTCCCGGGAGGCCAGCTGCAGCAGCCTGTGCTGCGGGCGG
GGCTATGACACCCAGAGCCGCCTGGTGGCCTTCTCCTGCCACTGCCAGGTGCAGTGGTGCTGCTACGTGGAGT
GCCAGCAATGTGTGCAGGAGGAGCTTGTGTACACCTGCAAGCAC

| | | | |
|---|---|---|---|
| NOV6n, SNP13381607 of | SEQ ID NO: 84 | | MW at 36422.2kD |
| CG50709-03, Protein Sequence | SNP Pos: 267 | 331 aa | SNP Change: Pro to Ser |

LTGREVLTPFPGLGTAAAPAQGGAHLKQCDLLKLSRRQKQLCRREPGLAETLRDAAHLGLLECQFQFRHERWN
CSLEGRTGLLKRGFKETAFLYAVSSAALTHTLARACSAGRMERCTCDDSPGLESRQAWQWGVCGDNLKYSTKF
LSNFLGSKRGNKDLRARADAHNTHVGIKAVKSGLRTTCKCHGVSGSCAVRTCWKQLSPFRETGQVLKLRYDSA
VKVSSATNEALGRLELWAPARQGSLTKGLAPRSGDLVYMEDSPSFCRSSKYSPGTAGRVCSREASCSSLCCGR
GYDTQSRLVAFSCHCQVQWCCYVECQQCVQEELVYTCKH

| | | |
|---|---|---|
| | SEQ ID NO: 85 | 993 bp |
| NOV6o, SNP13378336 of | ORF Start: at 1 | ORF Stop: end of sequence |
| CG50709-03, DNA Sequence | SNP Pos: 881 | SNP Change: A to G |

CTGACCGGGCCGGAAGTCCTGACGCCCTTCCCAGGATTGGGCACTGCGGCAGCCCCGGCACAGGGCGGGGCCC
ACCTGAAGCAGTGTGACCTGCTGAAGCTGTCCCGGCGGCAGAAGCAGCTCTGCCGGAGGGAGCCCGGCCTGGC
TGAGACCCTGAGGGATGCTGCGCACCTCGGCCTGCTTGAGTGCCAGTTTCAGTTCCGGCATGAGCGCTGGAAC
TGTAGCCTGGAGGGCAGGACGGGCCTGCTCAAGAGAGGCTTCAAAGAGACAGCTTTCCTGTACGCGGTGTCCT
CTGCCGCCCTCACCCACACCCTGGCCCGGGCCTGCAGCGCTGGGCGCATGGAGCGCTGCACCTGTGATGACTC
TCCGGGGCTGGAGAGCCGACAGGCCTGGCAATGGGGCGTGTGCGGTGACAACCTCAAGTACAGCACCAAGTTT
CTGAGCAACTTCCTGGGGTCCAAGAGAGGAAACAAGGACCTGCGGGCACGGGCAGACGCCCACAATACCCACG
TGGGCATCAAGGCTGTGAAGAGTGGCCTCAGGACCACGTGTAAGTGCCATGGCGTATCAGGCTCCTGTGCCGT
GCGCACCTGCTGGAAGCAGCTCTCCCCGTTCCGTGAGACGGGCCAGGTGCTGAAACTGCGCTATGACTCGGCT
GTCAAGGTGTCCAGTGCCACCAATGAGGCCTTGGGCCGCCTAGAGCTGTGGGCCCCTGCCAGGCAGGGCAGCC
TCACCAAAGGCCTGGCCCCAAGGTCTGGGGACCTGGTGTACATGGAGGACTCACCCAGCTTCTGCCGGCCCAG
CAAGTACTCACCTGGCACAGCAGGTAGGGTGTGCTCCCGGGAGGCCAGCTGCAGCAGCCTGTGCTGCGGGCGG
GGCTGTGACACCCAGAGCCGCCTGGTGGCCTTCTCCTGCCACTGCCAGGTGCAGTGGTGCTGCTACGTGCAGT
GCCAGCAATGTGTGCAGGAGGAGCTTGTGTACACCTGCAAGCAC

| | | | |
|---|---|---|---|
| NOV6o, SNP13378336 of | SEQ ID NO: 86 | | MW at 36372.2kD |
| CG50709-03, Protein Sequence | SNP Pos: 294 | 331 aa | SNP Change: Tyr to Cys |

LTGREVLTPFPGLGTAAAPAQGGAHLKQCDLLKLSRRQKQLCRREPGLAETLRDAAHLGLLECQFQFRHERWN
CSLEGRTGLLKRGFKETAFLYAVSSAALTHTLARACSAGRMERCTCDDSPGLESRQAWQWGVCGDNLKYSTKF
LSNFLGSKRGNKDLRARADAHNTHVGIKAVKSGLRTTCKCHGVSGSCAVRTCWKQLSPFRETGQVLKLRYDSA
VKVSSATNEALGRLELWAPARQGSLTKGLAPRSGDLVYMEDSPSFCRPSKYSPGTAGRVCSREASCSSLCCGR
GCDTQSRLVAFSCHCQVQWCCYVECQQCVQEELVYTCKH

| | | |
|---|---|---|
| | SEQ ID NO: 87 | 993 bp |
| NOV6p, SNP13378335 of | ORF Start: at 1 | ORF Stop: end of sequence |
| CG50709-03, DNA Sequence | SNP Pos: 977 | SNP Change: T to C |

CTGACCGGGCGGGAAGTCCTGACGCCCTTCCCAGGATTGGGCACTGCGGCAGCCCCGGCACAGGGCGGGGCCC
ACCTGAAGCAGTGTGACCTGCTGAAGCTGTCCCGGCGGCAGAAGCAGCTCTGCCGGAGGGAGCCCGGCCTGGC
TGAGACCCTGAGGGATGCTGCGCACCTCGGCCTGCTTGAGTGCCAGTTTCAGTTCCGGCATGAGCGCTGGAAC
TGTAGCCTGGAGGGCAGGACGGGCCTGCTCAAGAGAGGCTTCAAAGAGACAGCTTTCCTGTACGCGGTGTCCT
CTGCCGCCCTCACCCACACCCTGGCCCGGGCCTGCAGCGCTGGGCGCATGGAGCGCTGCACCTGTGATGACTC
TCCGGGGCTGGAGAGCCGACAGGCCTGGCAATGGGGCGTGTGCGGTGACAACCTCAAGTACAGCACCAAGTTT
CTGAGCAACTTCCTGGGGTCCAAGAGAGGAAACAAGGACCTGCGGGCACGGGCAGACGCCCACAATACCCACG
TGGGCATCAAGGCTGTGAAGAGTGGCCTCAGGACCACGTGTAAGTGCCATGGCGTATCAGGCTCCTGTGCCGT
GCGCACCTGCTGGAAGCAGCTCTCCCCGTTCCGTGAGACGGGCCAGGTGCTGAAACTGCGCTATGACTCGGCT
GTCAAGGTGTCCAGTGCCACCAATGAGGCCTTGGGCCGCCTAGAGCTGTGGGCCCCTGCCAGGCAGGGCAGCC
TCACCAAAGGCCTGGCCCCAAGGTCTGGGGACCTGGTGTACATGGAGGACTCACCCAGCTTCTGCCGGCCCAG
CAAGTACTCACCTGGCACAGCAGGTAGGGTGTGCTCCCGGGAGGCCAGCTGCAGCAGCCTGTGCTGCGGGCGG
GGCTATGACACCCAGAGCCGCCTGGTGGCCTTCTCCTGCCACTGCCAGGTGCAGTGGTGCTGCTACGTGGACT
GCCAGCAATGTGTGCAGGAGGAGCTTGCGTACACCTGCAAGCAC

| | | | |
|---|---|---|---|
| NOV6p, SNP13378335 of | SEQ ID NO: 88 | | MW at 36404.1kD |
| CG50709-03, Protein Sequence | SNP Pos: 326 | 331 aa | SNP Change: Val to Ala |

LTGREVLTPFPGLGTAAAPAQGGAHLKQCDLLKLSRRQKQLCRREPGLAETLRDAAHLGLLECQFQFRHERWN
CSLEGRTGLLKRGFKETAFLYAVSSAALTHTLARACSAGRMERCTCDDSPGLESRQAWQWGVCGDNLKYSTKF
LSNFLGSKRGNKDLRARADAHNTHVGIKAVKSGLRTTCKCHGVSGSCAVRTCWKQLSPFRETGQVLKLRYDSA
VKVSSATNEALGRLELWAPARQGSLTKGLAPRSGDLVYMEDSPSFCRPSKYSPGTAGRVCSREASCSSLCCGR
GYDTQSRLVAFSCHCQVQWCCYVECQQCVQEELAYTCKH

A ClustalW comparison of the above protein sequences yields the following sequence alignment shown in Table 6B.

TABLE 6B

| Comparison of the NOV6 protein sequences. | |
|---|---|
| NOV6a | ------------------------------LTGREVLTPFPGLGTAAAPAQGGAHLKQCD |
| NOV6b | ------------------------------------------------------TGSQCD |
| NOV6c | ----MRPPPALALAGLCLLALPAAAASYFGLTGREVLTPFPGLGTAAAPAQGGAHLKQCD |
| NOV6d | TGSTMRPPPALALAGLCLLALPAAAASYFGLTGREVLTPFPGLGTAAAPAQGGAHLKQCD |
| NOV6e | -----------------------TGSSYFGLTGREVLTPFPGLGTAAAPAQGGAHLKQCD |
| NOV6f | ------------------------------LTGREVLTPFPGLGTAAAPAQGGAHLKQCD |
| NOV6g | ------------------------------------------------------------ |
| NOV6h | --------------------------------------------------------KQCD |
| NOV6i | ----MRPPPALALAGLCLLALPAAAASYFGLTGREVLTPFPGLGTAAAPAQGGAHLKQCD |
| NOV6j | -----------------------TGSSYFGLTGREVLTPFPGLGTAAAPAQGGAHLKQCD |
| NOV6a | LLKLSRRQKQLCRREPGLAETLRDAAHLGLLECQFQFRHERWNCSLEGRTGLLKRGFKET |
| NOV6b | LLKLSRRQKQLCRREPGLAETLRDAAHLGLLECQFQFRHERWNCSLEGRTGLLKRGFKET |
| NOV6c | LLKLSRRQKQLCRREPGLAETLRDAAHLGLLECQFQFRHERWNCSLEGRTGLLKRGFKET |
| NOV6d | LLKLSRRQKQLCRREPGLAETLRDAAHLGLLECQFQFRHERWNCSLEGRTGLLKRGFKET |
| NOV6e | LLKLSRRQKQLCRREPGLAETLRDAAHLGLLECQFQFRHERWNCSLEGRTGLLKRGFKET |
| NOV6f | LLKLSRRQKQLCRREPGLAETLRDAAHLGLLECQFQFRHERWNCSLEGRMGLLKRGFKET |
| NOV6g | ------------------------------------------------------------ |
| NOV6h | LLKLSRRQKQLCRREPGLAETLRDAAHLGLLECQFQFRHERWNCSLEGRMGLLKRGFKET |
| NOV6i | LLKLSRRQKQLCRREPGLAETLRDAAHLGLLECQFQFRHERWNCSLEGRTGLLKRGFKET |
| NOV6j | LLKLSRRQKQLCRREPGLAETLRDAAHLGLLECQFQFRHERWNCSLEGRTGLLKRGFKET |
| NOV6a | AFLYAVSSAALTHTLARACSAGRMERCTCDDSPGLESRQAWQWGVCGDNLKYSTKFLSNF |
| NOV6b | AFLYAVSSAALTHTLARACSAGRMERCTCDDSPGLESRQAWQWGVCGDNLKYSTKFLSNF |
| NOV6c | AFLYAVSSAALTHTLARACSAGRMERCTCDDSPGLESRQAWQWGVCGDNLKYSTKFLSNF |
| NOV6d | AFLYAVSSAALTHTLARACSAGRMERCTCDDSPGLESRQAWQWGVCGDNLKYSTKFLSNF |
| NOV6e | AFLYAVSSAALTHTLARACSAGRMERCTCDDSPGLESRQAWQWGVCGDNLKYSTKFLSNF |
| NOV6f | AFLYAVSSAALTHTLARACSAGRMERCTCDDSPGLESRQAWQWGVCGDNLKYSTKFLSNF |
| NOV6g | -----------------------MERCTCDDSPGLESRQAWQWGVCGDNLKYSTKFLSNF |
| NOV6h | AFLYAVSSAALTHTLARACSAGRMERCTCDDSPGLESRQAWQWGVCGDNLKYSTKFLSNF |
| NOV6i | AFLYAVSSAALTHTLARACSAGRMERCTCDDSPGLESRQAWQWGVCGDNLKYSTKFLSNF |
| NOV6j | AFLYAVSSAALTHTLARACSAGRMERCTCDDSPGLESRQAWQWGVCGDNLKYSTKFLSNF |
| NOV6a | LGSKRGNKDLRARADAHNTHVGIKAVKSGLRTTCKCHGVSGSCAVRTCWKQLSPFRETGQ |
| NOV6b | LGSKRGNKDLRARADAHNTHVGIKAVKSGLRTTCKCHGVSGSCAVRTCWKQLSPFRETGQ |
| NOV6c | LGSKRGNKDLRARADAHNTHVGIKAVKSGLRTTCKCHGVSGSCAVRTCWKQLSPFRETGQ |
| NOV6d | LGSKRGNKDLRARADAHNTHVGIKAVKSGLRTTCKCHGVSGSCAVRTCWKQLSPFRETGQ |
| NOV6e | LGSKRGNKDLRARADAHNTHVGIKAVKSGLRTTCKCHGVSGSCAVRTCWKQLSPFRETGQ |
| NOV6f | LGSKRGNKDLRARADAHNTHVGIKAVKSGLRTTCKCHGVSGSCAVRTCWKQLSPFRETGQ |
| NOV6g | LGSKRGNKDLRARADAHNTHVGIKAVKSGLRTTCKCHGVSGSCAVRTCWKQLSPFRETGQ |
| NOV6h | LGSKRGNKDLRARADAHNTHVGIKAVKSGLRTTCKCHGVSGSCAVRTCWKQLSPFRETGQ |
| NOV6i | LGSKRGNKDLRARADAHNTHVGIKAVKSGLRTTCKCHGVSGSCAVRTCWKQLSPFRETGQ |
| NOV6j | LGSKRGNKDLRARADAHNTHVGIKAVKSGLRTTCKCHGVSGSCAVRTCWKQLSPFRETGQ |
| NOV6a | VLKLRYDSAVKVSSATNEALGRLELWAPARQGSLTKGLAPRSGDLVYMEDSPSFCRPSKY |
| NOV6b | VLKLRYDSAVKVSSATNEALGRLELWAPARQGSLTKGLAPRSGDLVYMEDSPSFCRPSKY |
| NOV6c | VLKLRYDSAVKVSSATNEALGRLELWAPARQGSLTKGLAPRSGDLVYMEDSPSFCRPSKY |
| NOV6d | VLKLRYDSAVKVSSATNEALGRLELWAPARQGSLTKGLAPRSGDLVYMEDSPSFCRPSKY |
| NOV6e | VLKLRYDSAVKVSSATNEALGRLELWAPARQGSLTKGLAPRSGDLVYMEDSPSFCRPSKY |
| NOV6f | VLKLRYDSAVKVSSATNEALGRLELWAPARQGSLTKGLAPRSGDLVYMEDSPSFCRPSKY |
| NOV6g | VLKLRYDSAVKVSSATNEALGRLELWAPARQGSLTKGLAPRSGDLVYMEDSPSFCRPSKY |
| NOV6h | VLKLRYDSAVKVSSATNEALGRLELWAPARQGSLTKGLAPRSGDLVYMEDSPSFCRPSKY |
| NOV6i | VLKLRYDSAVKVSSATNEALGRLELWAPARQGSLTKGLAPRSGDLVYMEDSPSFCRPSKY |
| NOV6j | VLKLRYDSAVKVSSATNEALGRLELWAPARQGSLTKGLAPRSGDLVYMEDSPSFCRPSKY |
| NOV6a | SPGTAGRVCSREASCSSLCCGRGYDTQSRLVAFSCHCQVQWCCYVECQQCVQEELVYTCK |
| NOV6b | SPGTAGRVCSREASCSSLCCGRGYDTQSRLVAFSCHCQVQWCCYVECQQCVQEELVYTCK |
| NOV6c | SPGTAGRVCSREASCSSLCCGRGYDTQSRLVAFSCHCQVQWCCYVECQQCVQEELVYTCK |
| NOV6d | SPGTAGRVCSREASCSSLCCGRGYDTQSRLVAFSCHCQVQWCCYVECQQCVQEELVYTCK |
| NOV6e | SPGTAGRVCSREASCSSLCCGRGYDTQSRLVAFSCHCQVQWCCYVECQQCVQEELVYTCK |
| NOV6f | SPGTAGRVCSREASCSSLCCCRGYDTQSRLVAFSCHCQVQWCCYVECQQCVQEELVYTCK |
| NOV6g | SPGTAGRVCSREASCSSLCCGRGYDTQSRLVAFSCHCQVQWCCYVECQQCVQEELVYTCK |
| NOV6h | SPGTAGRVCSREASCSSLCCGRGYDTQSRLVAFSCHCQV--------------------- |
| NOV6i | SPGTAGRVCSREASCSSLCCGRGYDTQSRLVAFSCHCQVQWCCYVECQQCVQEELVYTCK |
| NOV6j | SPGTAGRVCSREASCSSLCCGRGYDTQSRLVAFSCHCQVQWCCYVECQQCVQEELVYTCK |
| NOV6a | H--- |
| NOV6b | LEG- |
| NOV6c | H--- |
| NOV6d | HLEG |
| NOV6e | HLEG |
| NOV6f | H--- |
| NOV6g | H--- |
| NOV6h | ---- |
| NOV6i | HLEG |
| NOV6j | HLEG |
| NOV6a | (SEQ ID NO: 58) |
| NOV6b | (SEQ ID NO: 60) |
| NOV6c | (SEQ ID NO: 62) |
| NOV6d | (SEQ ID NO: 64) |
| NOV6e | (SEQ ID NO: 66) |
| NOV6f | (SEQ ID NO: 68) |
| NOV6g | (SEQ ID NO: 70) |
| NOV6h | (SEQ ID NO: 72) |
| NOV6i | (SEQ ID NO: 74) |
| NOV6j | (SEQ ID NO: 76) |

Further analysis of the NOV6a protein yielded the following properties shown in Table 6C.

TABLE 6C
Protein Sequence Properties NOV6a

| SignalP | No Known Signal Sequence Predicted | | | |
|---|---|---|---|---|
| analysis: | | | | |
| PSORT II | PSG: a new signal peptide prediction method | | | |
| analysis: | N-region: length 5; pos. chg 1; neg. chg 1 | | | |
| | H-region: length 21; peak value 7.18 | | | |
| | PSG score: 2.78 | | | |
| | GvH: von Heijne's method for signal seq. recognition | | | |
| | GvH score (threshold: −2.1): −5.38 | | | |
| | possible cleavage site: between 22 and 23 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 0 | | | |
| | number of TMS(s) . . . fixed | | | |
| | PERIPHERAL | Likelihood = 4.08 (at 90) | | |
| | ALOM score: 4.08 (number of TMSs: 0) | | | |
| | MTOP: Prediction of membrane topology (Hartmann et al.) | | | |
| | Center position for calculation: 6 | | | |
| | Charge difference: 3.5 C(4.5)-N(1.0) | | | |
| | C > N: C-terminal side will be inside | | | |
| | >>>Caution: Inconsistent mtop result with signal peptide | | | |
| | MITDISC: discrimination of mitochondrial targeting seq | | | |
| | R content: | 1 | Hyd Moment(75): | 11.91 |
| | Hyd Moment(95): | 8.21 | G content: | 5 |
| | D/E content: | 2 | S/T content: | 3 |

Score: −6.56
Gavel: prediction of cleavage sites for mitochondrial preseq
R-2 motif at 53 CRR|EP
NUCDISC: discrimination of nuclear localization signals
pat4: none
pat7: none
bipartite: none
content of basic residues: 14.2%
NLS Score: −0.47
KDEL: ER retention motif in the C-terminus: none
ER Membrane Retention Signals:
XXRR-like motif in the N-terminus: TGRE
KKXX-like motif in the C-terminus: YTCK
SKL: peroxisomal targeting signal in the C-terminus: none
PTS2: 2nd peroxisomal targeting signal: found
KLSRRQKQL at 33
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: nuclear
Reliability: 76.7
COIL: Lupas's algorithm to detect coiled-coil regions
total: 0 residues
Final Results (k = 9/23):
78.3%: nuclear
13.0%: mitochondrial
8.7%: cytoplasmic
>> prediction for CG50709-03 is nuc (k = 23)

A search of the NOV6a protein against the Geneseq database, a proprietary database that contains sequences published in patents and patent publication, yielded several homologous proteins shown in Table 6D.

TABLE 6D
Geneseq Results for NOV6a

| | | NOV6a | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| AAE17306 | Human WNT15 protein, | 1 . . . 331 | 330/331 (99%) | 0.0 |
| | sbg389686WNT15a #2 - Homo | 31 . . . 361 | 330/331 (99%) | |
| | sapiens, 361 aa. [WO200198342-A1, | | | |
| | 27 DEC. 2001] | | | |
| AAE17305 | Human WNT15 protein, | 1 . . . 331 | 330/331 (99%) | 0.0 |
| | sbg389686WNT15a #1 - Homo | 17 . . . 347 | 330/331 (99%) | |
| | sapiens, 704 aa. [WO200198342-A1, | | | |
| | 27 DEC. 2001] | | | |
| ABB77769 | Amino acid sequence of human Wnt | 1 . . . 331 | 330/331 (99%) | 0.0 |
| | (Zwnt5) polypeptide variant - Homo | 4 . . . 334 | 330/331 (99%) | |
| | sapiens, 334 aa. [WO200231148-A2, | | | |
| | 18 APR. 2002] | | | |
| ABB77768 | Amino acid sequence of human Wnt | 1 . . . 331 | 330/331 (99%) | 0.0 |
| | (Zwnt5) polypeptide - Homo sapiens, | 31 . . . 361 | 330/331 (99%) | |
| | 361 aa. [WO200231148-A2, | | | |
| | 18 APR. 2002] | | | |
| ABB83080 | Wnt family related protein 2 - Homo | 1 . . . 331 | 330/331 (99%) | 0.0 |
| | sapiens, 363 aa. [WO200250278-A2, | 33 . . . 363 | 330/331 (99%) | |
| | 27 JUN. 2002] | | | |

In a BLAST search of public sequence databases, the NOV6a protein was found to have homology to the proteins shown in the BLASTP data in Table 6E.

TABLE 6E
Public BLASTP Results for NOV6a

| | | NOV6a | | |
|---|---|---|---|---|
| Protein | | Residues/ | Identities/ | |
| Accession | | Match | Similarities for the | Expect |
| Number | Protein/Organism/Length | Residues | Matched Portion | Value |
| O14905 | Wnt-9b protein precursor (Wnt-15) | 1 . . . 331 | 331/331 (100%) | 0.0 |
| | (Wnt-14b) - Homo sapiens (Human), | 27 . . . 357 | 331/331 (100%) | |
| | 357 aa. | | | |
| Q8C718 | WNT14B - Mus musculus (Mouse), | 1 . . . 330 | 310/330 (93%) | 0.0 |
| | 359 aa. | 29 . . . 358 | 319/330 (95%) | |
| O35468 | Wnt-9b protein precursor (Wnt-15) | 1 . . . 330 | 310/330 (93%) | 0.0 |
| | (Wnt-14b) - Mus musculus (Mouse), | 29 . . . 358 | 319/330 (95%) | |
| | 359 aa. | | | |
| O14904 | Wnt-9a protein precursor (Wnt-14) - | 1 . . . 330 | 209/335 (62%) | e−124 |
| | Homo sapiens (Human), 365 aa. | 33 . . . 364 | 255/335 (75%) | |
| Q8R5M2 | Wnt-9a protein precursor (Wnt-14) - | 1 . . . 330 | 208/335 (62%) | e−123 |
| | Mus musculus (Mouse), 365 aa. | 33 . . . 364 | 255/335 (76%) | |

PFam analysis predicts that the NOV6a protein contains the domains shown in the Table 6F.

TABLE 6F
Domain Analysis of NOV6a

| | | Identities/ | |
|---|---|---|---|
| Pfam | | Similarities | Expect |
| Domain | NOV6a Match Region | for the Matched Region | Value |
| wnt | 28 . . . 330 | 132/354 (37%) | 2.1e−104 |
| | | 234/354 (66%) | |

### Example 7

The NOV7 clone was analyzed, and the nucleotide and encoded polypeptide sequences are shown in Table 7A.

TABLE 7A
NOV7 Sequence Analysis

| | | |
|---|---|---|
| NOV7a, CG53054-02 | SEQ ID NO: 89 | 1128 bp |
| DNA Sequence | ORF Start: ATG at 31 | ORF Stop: TGA at 1102 |

TCCCGGCCCTCCGCGCCCTCTCGCGCGGCGATGGCCCCACTCGGATACTTCTTACTCCTCTGCAGCCTGAAGC
AGGCTCTGGGCAGCTACCCGATCTGGTGGCTGACGGGCAGCGAGCCCCTGACCATCCTCCCGCTGACCCTGGA
GCCAGAGGCGGCTGCCCAGGCGCACTACAAGGCCTGCGACCGGCTGAAGCTGGAGCGGAAGCAGCGGCGCATG
TGCCGCCGGGACCCGGGCGTGGCAGAGACGCTGGTGGAGGCCGTGAGCATGAGTGCGCTCGAGTGCCAGTTCC
AGTTCCGCTTTGAGCGCTGGAACTGCACGCTGGAGGGCCGCTACCGGGCCAGCCTGCTCAAGCGAGGTTTCAA
GGAGACTGCCTTCCTCTATGCCATCTCCTCGGCTGGCCTGACGCACGCACTGGCCAAGGCGTGCAGCGCGGGC
CGCATGGAGCGCTGTACCTGCGATGAGGCACCCGACCTGGAGAACCGTGAGGCCTGGCAGTGGGGGGGCTGCG
GAGACAACCTTAAGTACAGCAGCAAGTTCGTCAAGGAATTCCTGGGCAGACGGTCAAGCAAGGATCTGCGAGC
CCGTGTGGACTTCCACAACAACCTCGTGGGTGTGAAGGTGATCAAGGCTGGGGTGGAGACCACCTGCAAGTGC
CACGGCGTGTCAGGCTCATGCACGGTGCGGACCTGCTGGCGGCAGTTGGCGCCTTTCCATGAGGTGGGCAAGC
ATCTGAAGCACAAGTATGAGACGGCACTCAAGGTGGGCAGCACCACCAATGAAGCTGCCGGCGAGGCAGGTGC
CATCTCCCCACCACGGGGCCGTGCCTCGGGGGCAGGTGGCAGCGACCCGCTGCCCCGCACTCCAGAGCTGGTG
CACCTGGATGACTCGCCTAGCTTCTGCCTGGCTGGCCGCTTCTCCCCGGGCACCGCTGGCCGTAGGTGCCACC
GTGAGAAGAACTGCGAGAGCATCTGCTGTGGCCGCGGCCATAACACACAGAGCCGGGTGGTGACAAGGCCCTG
CCAGTGCCAGGTGCGTTGGTGCTGCTATGTGGAGTGCAGGCAGTGCACGCAGCGTGAGGAGGTCTACACCTGC
AAGGGCTGAGTTCCCAGGCCCTGCCAGCCCTGC

| | | | |
|---|---|---|---|
| NOV7a, CG53054-02 | | | |
| Protein Sequence | SEQ ID NO: 90 | 357 aa | MW at 39756.1kD |

MAPLGYFLLLCSLKQALGSYPIWWLTGSEPLTILPLTLEPEAAAQAHYKACDRLKLERKQRRMCRRDPGVAET
LVEAVSMSALECQFQFRFERWNCTLEGRYRASLLKRGFKETAFLYAISSAGLTHALAKACSAGRMERCTCDEA
PDLENREAWQWGGCGDNLKYSSKFVKEFLGRRSSKDLRARVDFHNNLVGVKVIKAGVETTCKCHGVSGSCTVR
TCWRQLAPFHEVGKHLKHKYETALKVGSTTNEAAGEAGAISPPRGRASGAGGSDPLPRTPELVHLDDSPSFCL
AGRFSPGTAGRRCHREKNCESICCGRGHNTQSRVVTRPCQCQVRWCCYVECRQCTQREEVYTCKG

| | | |
|---|---|---|
| NOV7b, 170251039 | SEQ ID NO: 91 | 1029 bp |
| DNA Sequence | ORF Start: at 1 | ORF Stop: end of sequence |

GGATCCAGCTACCCGATCTGGTGGCTGACGGGCAGCGAGCCCCTGACCATCCTCCCGCTGACCCTGGAGCCAG
AGGCGGGCGCCCAGGCGCACTACAAGGCCTGCGACCGGCTGAAGCTGGAGCGGAAGCAGCGGCGCATGTGCCG
CCGGGACCCGGGCGTGGTAGAGACGCTGGTGGAGGCCGTGAGCATGAGTGCGCTCGAGTGCCAGTTCCAGTTC
CGCTTTGAGCGCTGGAACTGCACGCTGGAGGGCCGCTACCGGGCCAGCCTGCTCAAGCGAGGCTTCAAGGAGA
CTGCCTTCCTCTATGCCATCTCCTCGGCTGGCCTGACGCACGCACTGGCCAAGGCGTGCAGCGCGGGCCGCAT
GGAGCGCTGTACCTGCGATGAGGCACCCGACCTGGAGAACCGTGAGGCCTGGCAGTGGGGGGGCTGCGGAGAC
AACCTTAAGTACAGCAGCAAGTTCGTCAAGGAATTCCTGGGCAGACGGTCAAGCAAGCATCTGCGAGCCCGTG
TGCACTTCCACAACAACCTCGTGGGTGTGAAGGTGATCAAGGCTGGGGTGGAGACCACCTGCAAGTGCCACGG
CGTGTCAGGCTCATGCACGGTGCGGACCTGCTGGCGGCAGTTGGCGCCTTTCCATGAGGTGGGCAAGCATCTG
AAGCACAAGTATGAGACGGCACTCAAGGTGGGCAGCACCACCAATGAAGCTGCCGGCGAGGCAGGTGCCATCT
CCCCACCACGGGGCCGTGCCTCGGGGGCAGGTGGCAGCGACCCGCTGCCCCGCACTCCAGAGCTGGTGCACCT
GGATGACTCGCCTAGCTTCTGCCTGGCTGGCCGCTTCTCCCCGGGCACCGCTGGCCGTAGGTGCCACCGTGAG
AAGAACTGCGAGAGCATCTGCTGTGGCCGCGGCCATAACACACAGAGCCGGGTGGTGACAAGGCCCTGCCAGT
GCCAGGTGCGTTGGTGCTGCTATGTGGAGTGCAGGCAGTGCACGCAGCGTGAGGAGGTCTACACCTGCAAGGG
CGTCGAC

| | | | |
|---|---|---|---|
| NOV7b, 170251039 | | | |
| Protein Sequence | SEQ ID NO: 92 | 343 aa | MW at 38208.1kD |

GSSYPIWWLTGSEPLTILPLTLEPEAGAQAHYKACDRLKLERKQRRMCRRDPGVVETLVEAVSMSALECQFQF
RFERWNCTLEGRYRASLLKRGFKETAFLYAISSAGLTHALAKACSAGRMERCTCDEAPDLENREAWQWGGCGD
NLKYSSKFVKEFLGRRSSKDLRARVDFHNNLVGVKVIKAGVETTCKCHGVSGSCTVRTCWRQLAPFHEVGKHL
KHKYETALKVGSTTNEAAGEAGAISPPRGRASGAGGSDPLPRTPELVHLDDSPSFCLAGRFSPGTAGRRCHRE
KNCESICCGRGHNTQSRVVTRPCQCQVRWCCYVECRQCTQREEVYTCKGVD

| | | |
|---|---|---|
| NOV7c, 170251076 | SEQ ID NO: 93 | 1029 bp |
| DNA Sequence | ORF Start: at 1 | ORF Stop: end of sequence |

GGATCCAGCTACCCGATCTGGTGGCTGACGGGCAGCGACCCCCTGACCATCCTCCCGCTGACCCTGGAGCCAG
AGGCGGCCGCCCAGGCGCACTACAAGGCCTGCGACCGGCTGAAGCTGGAGCGGAAGCAGCGGCGCATGTGCCG
CCCGGACCCGGGCGTGGCAGAGACGCTGGTGGAGGCCGTGAGCATGAGTGCGCTCGAGTGCCAGTTCCAGTTC
CGCTTTGAGCGCTGGAACTGCACGCTGGAGGGCCGCTACCGGGCCAGCCTGCTCAAGCGAGGCTTCAAGGAGA
CTGCCTTCCTCTATGCCATCTCCTCGGCTGGCCTGACGCACGCACTGGCCAAGGCGTGCAGCGCGGGCCGCAT
GGAGCGCTGTACCTGCGATGAGGCACCCGACCTGGAGAACCGTGAGGCCTGGCAGTGGGGGGGCTGCGGAGAC
AACCTTAAGTACAGCAGCAAGTTCGTCAAGGAATTCCTGGGCAGACGGTCAAGCAAGGATCTGCGAGCCCGTG
TGGACTTCCACAACAACCTCGTGGGTGTGAAGGTGATCAAGGCTGGGGTGGAGACCACCTGCAAGTGCCACGG
CGTGTCAGGCTCATGCACGGTGCGGACCTGCTGGCGGCAGTTGGCGCCTTTCCATGAGGTGGGCAAGCATCTG
AAGCACAAGTATGAGACGGCACTCAAGGTGGGCAGCACCACCAATGAAGCTGCCGGCGAGGCAGGTGCCATCT
CCCCACCACGGGGCCGTGCCTCGGGGGCAGGTGGCAGCGACCCGCTGCCCCGCACTCCAGAGCTGGTGCACCT
GGATGACTCGCCTAGCTTCTGCCTGGCTGGCCGCTTCTCCCCGGGCACCGCTGGCCGTAGGTGCCACCGTGAG
AAGAACTGCGAGAGCATCTGCTGTGGCCGCGGCCATAACACACAGAGCCGGGTGGTGACAAGGCCCTGCCAGT
GCCAGGTGCGTTGGTGCTGCTATGTGGAGTGCAGGCAGTGCACGCAGCGTGAGGAGGTCTACACCTGCAAGGG
CGTCGAC

| | | | |
|---|---|---|---|
| NOV7c, 170251076 | | | |
| Protein Sequence | SEQ ID NO: 94 | 343 aa | MW at 38194.1kD |

GSSYPIWWLTGSEPLTILPLTLEPEAAAQAHYKACDRLKLERKQRRMCRRDPGVAETLVEAVSMSALECQFQF
RFERWNCTLEGRYRASLLKRGFKETAFLYAISSAGLTHALAKACSAGRMERCTCDEAPDLENREAWQWGGCGD
NLKYSSKFVKEFLGRRSSKDLRARVDFHNNLVGVKVIKAGVETTCKCHGVSGSCTVRTCWRQLAPFHEVGKHL
KHKYETALKVGSTTNEAAGEAGAISPPRGRASGAGGSDPLPRTPELVHLDDSPSFCLAGRFSPGTAGRRCHRE
KNCESICCGRGHNTQSRVVTRPCQCQVRWCCYVECRQCTQREEVYTCKGVD

| | | |
|---|---|---|
| NOV7d, CG53054-01 | SEQ ID NO: 95 | 1085 bp |
| DNA Sequence | ORF Start: ATG at 13 | ORF Stop: TGA at 1078 |

TAGTGAGCCGAGATGGCACTACTATATTCCAGCTTGGGTGTGGTTGTGTGCACCTGTAGTCCTAGTTACTTTG
GACTGACGGGCAGCGAGCCCCTGACCATCCTCCCGCTGACCCTGGAGCCAGAGGCGGCTGCCCAGGCGCACTA
CAAGGCCTGCGACCGGCTGAAGCTGGAGCGGAAGCAGCGGCGCATGTGCCGCCGGGACCCGGGCGTGGCAGAG
ACGCTGGTGGAGGCCGTGAGCATGAGTGCGCTCGAGTGCCAGTTCCAGTTCCGCTTTGAGCGCTGGAACTGCA
CGCTGGAGGGCCGCTACCGGGCCAGCCTGCTCAAGCGAGGTTTCAAGGAGACTGCCTTCCTCTATGCCATCTC
CTCGGCTGGCCTGACGCACGCACTGGCCAAGGCGTGCAGCGCGGGCCGCATGGAGCGCTGTACCTGCGATGAG
GCACCCGACCTGGAGAACCGTGAGGGCTGGAAGTGGGGTGGCTGTAGCGAGGACATCGAGTTTGGTGGGATGG
TGTCTCGGGAGTTCGCCGACGCCCGGGAGAACCGGCCAGATGCCCGCTCAGCCATGAACCGCCACAACAACGA
GGCTGGGCGCCAGGTGATCAAGGCTGGGGTGGAGACCACCTGCAAGTGCCACGGCGTGTCAGGCTCATGCACG
GTGCGGACCTGCTGGCGGCAGTTGGCGCCTTTCCATGAGGTGGGCAAGCATCTGAAGCACAAGTATGAGTCGG
CACTCAAGGTGGGCAGCACCACCAATGAAGCTGCCGGCGAGGCAGGTGCCATCTCCCCACCACGGGGCCGTGC
CTCGGGGGCAGGTGGCAGCGACCCGCTGCCCCGCACTCCAGAGCTGGTGCACCTGGATGACTCGCCTAGCTTC
TGCCTGGCTGGCCGCTTCTCCCCGGGCACCGCTGGCCGTAGGTGCCACCGTGAGAAGAACTGCGAGAGCATCT
GCTGTGGCCGCGGCCATAACACACAGAGCCGGGTGGTGACAAGGCCCTGCCAGTGCCAGGTGCGTTGGTGCTG
CTATGTGGAGTGCAGGCAGTGCACGCAGCGTGAGGAGGTCTACACCTGCAAGGGCTGAGTTCC

| | | | |
|---|---|---|---|
| NOV7d, CG53054-01 | | | |
| Protein Sequence | SEQ ID NO: 96 | 355 aa | MW at 39194.1kD |

MALLYSSLGVVVCTCSPSYFGLTGSEPLTILPLTLEPEAAAQAHYKACDRLKLERKQRRMCRRDPGVAETLVE
AVSMSALECQFQFRFERWNCTLEGRYRASLLKRGFKETAFLYAISSAGLTHALAKACSAGRMERCTCDEAPDL
ENREGWKWGGCSEDIEFGGMVSREFADARENRPDARSAMNRHNNEAGRQVIKAGVETTCKCHGVSGSCTVRTC
WRQLAPFHEVGKHLKHKYESALKVGSTTNEAAGEAGAISPPRGRASGAGGSDPLPRTPELVHLDDSPSFCLAG
RFSPGTAGRRCHREKNCESICCGRGHNTQSRVVTRPCQCQVRWCCYVECRQCTQREEVYTCKG

| | | |
|---|---|---|
| NOV7e, CG53054-03 | SEQ ID NO: 97 | 1029 bp |
| DNA Sequence | ORF Start: at 7 | ORF Stop: at 1024 |

GGATCCAGCTACCCGATCTGGTGGCTGACGGGCAGCGAGCCCCTGACCATCCTCCCGCTGACCCTGGAGCCAG
AGGCGGCCGCCCAGGCGCACTACAAGGCCTGCGACCGGCTGAAGCTGGAGCGGAAGCAGCGGCGCATGTGCCG
CCGGGACCCGGGCGTGGCAGAGACGCTGGTGGAGGCCGTGAGCATGAGTGCGCTCGAGTGCCAGTTCCAGTTC
CGCTTTGAGCGCTGGAACTGCACGCTGGAGGGCCGCTACCGGGCCAGCCTGCTCAAGCGAGGCTTCAAGGAGA
CTGCCTTCCTCTATGCCATCTCCTCGGCTGGCCTGACGCACGCACTGGCCAAGGCGTGCAGCGCGGGCCGCAT
GGAGCGCTGTACCTGCGATGAGGCACCCGACCTGGAGAACCGTGAGGCCTGGCAGTGGGGGGGCTGCGGAGAC
AACCTTAAGTACAGCAGCAAGTTCGTCAAGGAATTCCTGGGCAGACGGTCAAGCAAGGATCTGCGAGCCCGTG
TGGACTTCCACAACAACCTCGTGGGTGTGAAGGTGATCAAGGCTGGGGTGGAGACCACCTGCAAGTGCCACGG
CGTCTCAGGCTCATGCACGGTGCGGACCTGCTGGCGGCAGTTGGCGCCTTTCCATGAGGTGGGCAAGCATCTG
AAGCACAAGTATGAGACGGCACTCAAGGTGGGCAGCACCACCAATGAAGCTGCCGGCGAGGCAGGTGCCATCT
CCCCACCACGGGGCCGTGCCTCGGGGGCAGGTGGCAGCGACCCGCTGCCCCGCACTCCAGAGCTGGTGCACCT
GGATGACTCGCCTAGCTTCTGCCTGGCTGGCCGCTTCTCCCCGGGCACCGCTGGCCGTAGGTGCCACCGTGAG
AAGAACTGCGAGAGCATCTGCTGTGGCCGCGGCCATAACACACAGAGCCGGGTGGTCACAAGGCCCTGCCAGT
GCCAGGTGCGTTGGTGCTGCTATGTGGAGTGCAGGCAGTGCACGCAGCGTGAGGAGGTCTACACCTGCAAGGG
CGTCGAC

| | | | |
|---|---|---|---|
| NOV7e, CG53054-03 | | | |
| Protein Sequence | SEQ ID NO: 98 | 339 aa | MW at 37835.8kD |

SYPIWWLTGSEPLTILPLTLEPEAAAQAHYKACDRLKLERKQRRMCRRDPGVAETLVEAVSMSALECQFQFRF
ERWNCTLEGRYRASLLKRGFKETAFLYAISSAGLTHALAKACSAGRMERCTCDEAPDLENREAWQWGGCGDNL
KYSSKFVKEFLGRRSSKDLRARVDFHNNLVGVKVIKAGVETTCKCHGVSGSCTVRTCWRQLAPFHEVGKHLKH
KYETALKVGSTTNEAAGEAGAISPPRGRASGAGGSDPLPRTPELVHLDDSPSFCLAGRFSPGTAGRRCHREKN
CESICCGRGHNTQSRVVTRPCQCQVRWCCYVECRQCTQREEVYTCKG

| | | |
|---|---|---|
| NOV7f, CG53054-04 | SEQ ID NO: 99 | 1631 bp |
| DNA Sequence | ORF Start: ATG at 12 | ORF Stop: TGA at 1107 |

GGCGCGGCAAGATGCTGGATGGGTCCCCGCTGGCGCGCTGGCTGGCCGCGGCCTTCGGGCTGACGCTGCTGCT
CGCCGCGCTGCGCCCTTCGGCCGCCTACTTCGGGCTGACGGGCAGCGAGCCCCTGACCATCCTCCCGCTGACC
CTGGAGCCAGAGGCGGCCGCCCAGGCGCACTACAAGGCCTGCGACCGGCTGAAGCTGCAGCGGAAGCAGCGGC
GCATGTGCCGCCGGGACCCGGGCGTGGCAGAGACGCTGGTGGAGGCCGTGAGCATGAGTGCGCTCGAGTGCCA
GTTCCAGTTCCGCTTTGAGCGCTGGAACTGCACGCTGGAGGGCCGCTACCGGGCCAGCCTGCTCAAGCGAGGC
TTCAAGGAGACTGCCTTCCTCTATGCCATCTCCTCGGCTGGCCTGACGCACGCACTGGCCAAGGCGTGCAGCG
CGGGCCGCATGGAGCGCTGTACCTGCGATGAGGCACCCGACCTGGAGAACCGTGAGGCCTGGCAGTGGGGGGG
CTGCGGAGACAACCTTAAGTACAGCAGCAAGTTCGTCAAGGAATTCCTGGGCAGACGGTCAACCAAGGATCTG
CGAGCCCGTGTGGACTTCCACAACAACCTCGTGGGTGTGAAGGTGATCAAGGCTGGGGTGGAGACCACCTGCA
AGTGCCACGGCGTGTCAGGCTCATGCACGGTGCGGACCTGCTGGCGGCAGTTGGCGCCTTTCCATGAGGTGGG
CAAGCATCTGAAGCACAAGTATGAGACGGCACTCAAGGTGGGCAGCACCACCAATGAAGCTGCCGGCGAGGCA
GGTGCCATCTCCCCACCACGGGGCCGTGCCTCGGGGGCAGGTGGCAGCGACCCGCTGCCCCGCACTCCAGAGC
TGGTGCACCTGGATGACTCGCCTAGCTTCTGCCTGGCTGGCCGCTTCTCCCCGGGCACCGCTGGCCGTAGGTG
CCACCGTGAGAAGAACTGCGAGAGCATCTGCTGTGGCCGCGGCCATAACACACAGAGCCGGGTGGTGACAAGG
CCCTGCCAGTGCCAGGTGCGTTGGTGCTGCTATGTGGAGTGCAGGCAGTGCACGCAGCGTGAGGAGGTCTACA
CCTGCAAGGGCTGACTTCCCAGGCCCTGCCAGCCCTGCTGCACAGGCTGCAGGCATTGCACACGGTGTGAAGG
GTCTACACCTGCACAGGCTGAGTTCCTGGGCTCGACCAGCCCAGCTGCGTGGGGTACAGGCATTGCACACACT
GTGAATGGGTCTACACCTGCATGGGCTGAGTCCCTGGGCTCAGACCTAGCAGCGTGGGGTAGTCCCTGGGCTC
AGTCCTAGCTGCATGGGGTGCAGGCATTGCACAGAGCATGAATGGGCCTACACCTGCCAAGGCTGAATCCCTG
GGCCCAGCCAGCCCTGCTGCACATGGCACAGGCATTGCACACGGTGTGAGGAGTGTACACCTGCAAGGGCTGA
GGCCCTGGGCCCAGTCAGCCCTGCTGCTCAGAGTGCAGGCATTGCACATGGTGTGAGAAGGTCTACACCTGCA
AGGGACGAGTCCCCGGGCCTGGCCAACCCTGCTGTGCAGGGTGAGGGCCATGCATGCTAGTATGAGGGGTCTA
CACCTGCAAGGACTGAGAGGCTTTT

| | | | |
|---|---|---|---|
| NOV7f, CG53054-04 | | | |
| Protein Sequence | SEQ ID NO: 100 | 365 aa | MW at 40319.7kD |

MLDGSPLARWLAAAFGLTLLLAALRPSAAYFGLTGSEPLTILPLTLEPEAAAQAHYKACDRLKLERKQRRMCR
RDPGVAETLVEAVSMSALECQFQFRFERWNCTLEGRYRASLLKRGFKETAFLYAISSAGLTHALAKACSAGRM
ERCTCDEAPDLENREAWQWGGCGDNLKYSSKFVKEFLGRRSSKDLRARVDFHNNLVGVKVIKAGVETTCKCHG
VSGSCTVRTCWRQLAPFHEVGKHLKHKYETALKVGSTTNEAAGEAGAISPPRGRASGAGGSDPLPRTPELVHL
DDSPSFCLAGRFSPGTAGRRCHREKNCESICCGRGHNTQSRVVTRPCQCQVRWCCYVECRQCTQREEVYTCKG

A ClustalW comparison of the above protein sequences yields the following sequence alignment shown in Table 7B.

TABLE 7B
Comparison of the NOV7 protein sequences.

| | |
|---|---|
| NOV7a | --------MAPLGYFLLLCSLKQALGSYPIWWLTGSEPLTILPLTLEPEAAAQAHYKACD |
| NOV7b | ------------------------GSSYPIWWLTGSEPLTILPLTLEPEAGAQAHYKACD |
| NOV7c | ------------------------GSSYPIWWLTGSEPLTILPLTLEPEAAAQAHYKACD |
| NOV7d | -----------MALLYSSLGVVVCTCSPSYFGLTGSEPLTILPLTLEPEAAAQAHYKACD |
| NOV7e | --------------------------SYPIWWLTGSEPLTILPLTLEPEAAAQAHYKACD |
| NOV7f | MLDGSPLARWLAAAFGLTLLLAALRPSAAYFGLTGSEPLTILPLTLEPEAAAQAHYKACD |
| NOV7a | RLKLERKQRRMCRRDPGVAETLVEAVSMSALECQFQFRFERWNCTLEGRYRASLLKRGFK |
| NOV7b | RLKLERKQRRMCRRDPGVVETLVEAVSMSALECQFQFRFERWNCTLEGRYRASLLKRGFK |
| NOV7c | RLKLERKQRRMCRRDPGVAETLVEAVSMSALECQFQFRFERWNCTLEGRYRASLLKRGFK |
| NOV7d | RLKLERKQRRMCRRDPGVAETLVEAVSMSALECQFQFRFERWNCTLEGRYRASLLKRGFK |
| NOV7e | RLKLERKQRRMCRRDPGVAETLVEAVSMSALECQFQFRFERWNCTLEGRYRASLLKRGFK |
| NOV7f | RLKLERKQRRMCRRDPGVAETLVEAVSMSALECQFQFRFERWNCTLEGRYRASLLKRGFK |
| NOV7a | ETAFLYAISSAGLTHALAKACSAGRMERCTCDEAPDLENREAWQWGGCGDNLKYSSKFVK |
| NOV7b | ETAFLYAISSAGLTHALAKACSAGRMERCTCDEAPDLENREAWQWGGCGDNLKYSSKFVK |
| NOV7c | ETAFLYAISSAGLTHALAKACSAGRMERCTCDEAPDLENREAWQWGGCGDNLKYSSKFVK |
| NOV7d | ETAFLYAISSAGLTHALAKACSAGRMERCTCDEAPDLENREGWKWGGCSEDIEFGGMVSR |
| NOV7e | ETAFLYAISSAGLTHALAKACSAGRMERCTCDEAPDLENREAWQWGGCGDNLKYSSKFVK |
| NOV7f | ETAFLYAISSAGLTHALAKACSAGRMERCTCDEAPDLENREAWQWGGCGDNLKYSSKFVK |
| NOV7a | EFLGRRSSK-DLRARVDFHNNLVGVKVIKAGVETTCKCHGVSGSCTVRTCWRQLAPFHEV |
| NOV7b | EFLGRRSSK-DLRARVDFHNNLVGVKVIKAGVETTCKCHGVSGSCTVRTCWRQLAPFHEV |
| NOV7c | EFLGRRSSK-DLRARVDFHNNLVGVKVIKAGVETTCKCHGVSGSCTVRTCWRQLAPFHEV |
| NOV7d | EFADARENRPDARSAMNRHNNEAGRQVIKAGVETTCKCHGVSGSCTVRTCWRQLAPFHEV |
| NOV7e | EFLGRRSSK-DLRARVDFHNNLVGVKVTKAGVETTCKCHGVSGSCTVRTCWRQLAPFHEV |
| NOV7f | EFLGRRSSK-DLRARVDFHNNLVGVKVIKAGVETTCKCHGVSGSCTVRTCWRQLAPFHEV |
| NOV7a | GKHLKHKYETALKVGSTTNEAAGEAGAISPPRGRASGAGGSDPLPRTPELVHLDDSPSFC |
| NOV7b | GKHLKHKYETALKVGSTTNEAAGEAGAISPPRGRASGAGGSDPLPRTPELVHLDDSPSFC |
| NOV7c | GKHLKHKYETALKVGSTTNEAAGEAGAISPPRGRASGAGGSDPLPRTPELVHLDDSPSFC |
| NOV7d | GKHLKHKYESALKVGSTTNEAAGEAGAISPPRGRASGAGGSDPLPRTPELVHLDDSPSFC |
| NOV7e | GKHLKHKYETALKVGSTTNEAAGEAGAISPPRGRASGAGGSDPLPRTPELVHLDDSPSFC |
| NOV7f | GKHLKHKYETALKVGSTTNEAAGEAGAISPPRGRASGAGGSDPLPRTPELVHLDDSPSFC |
| NOV7a | LAGRFSPGTAGRRCHREKNCESICCGRGHNTQSRVVTRPCQCQVRWCCYVECRQCTQREE |
| NOV7b | LAGRFSPGTAGRRCHREKNCESICCGRGHNTQSRVVTRPCQCQVRWCCYVECRQCTQREE |
| NOV7c | LAGRFSPGTAGRRCHREKNCESICCGRGHNTQSRVVTRPCQCQVRWCCYVECRQCTQREE |
| NOV7d | LAGRFSPGTAGRRCHREKNCESICCGRGHNTQSRVVTRPCQCQVRWCCYVECRQCTQREE |
| NOV7e | LAGRFSPGTAGRRCHREKNCESICCGRGHNTQSRVVTRPCQCQVRWCCYVECRQCTQREE |
| NOV7f | LAGRFSPGTAGRRCHREKNCESICCGRGHNTQSRVVTRPCQCQVRWCCYVECRQCTQREE |
| NOV7a | VYTCKG-- |
| NOV7b | VYTCKGVD |
| NOV7c | VYTCKGVD |
| NOV7d | VYTCKG-- |
| NOV7e | VYTCKG-- |
| NOV7f | VYTCKG-- |
| NOV7a | (SEQ ID NO: 90) |
| NOV7b | (SEQ ID NO: 92) |
| NOV7c | (SEQ ID NO: 94) |
| NOV7d | (SEQ ID NO: 96) |
| NOV7e | (SEQ ID NO: 98) |
| NOV7f | (SEQ ID NO: 100) |

Further analysis of the NOV7a protein yielded the following properties shown in Table 7C.

TABLE 7C
Protein Sequence Properties NOV7a

| SignalP | Cleavage site between residues 19 and 20 | | | |
|---|---|---|---|---|
| analysis: | | | | |
| PSORT II | PSG: a new signal peptide prediction method | | | |
| analysis: | N-region: length 0; pos. chg 0; neg. chg 0 | | | |
| | H-region: length 13; peak value 9.00 | | | |
| | PSG score: 4.60 | | | |
| | GvH: von Heijne's method for signal seq. recognition | | | |
| | GvH score (threshold: −2.1): 0.73 | | | |
| | possible cleavage site: between 18 and 19 | | | |
| | >>> Seems to have a cleavable signal peptide (1 to 18) | | | |
| | ALOM: Klein et al's method for TM region allocation | | | |
| | Init position for calculation: 19 | | | |
| | Tentative number of TMS(s) for the threshold 0.5: 0 | | | |
| | number of TMS(s) . . . fixed | | | |
| | PERIPHERAL | Likelihood = 3.76 (at 114) | | |
| | ALOM score: 3.76 (number of TMSs: 0) | | | |
| | MTOP: Prediction of membrane topology (Hartmann et al.) | | | |
| | Center position for calculation: 9 | | | |
| | Charge difference: 0.0 C(1.0)-N(1.0) | | | |
| | N >= C: N-terminal side will be inside | | | |
| | MITDISC: discrimination of mitochondrial targeting seq | | | |
| | R content: | 0 | Hyd Moment(75): | 1.56 |
| | Hyd Moment(95): | 3.50 | G content: | 3 |
| | D/E content: | 1 | S/T content: | 4 |

Score: −6.15
Gavel: prediction of cleavage sites for mitochondrial preseq
cleavage site motif not found
NUCDISC: discrimination of nuclear localization signals
pat4: none
pat7: none
bipartite: none
content of basic residues: 14.8%
NLS Score: −0.47
KDEL: ER retention motif in the C-terminus: none
ER Membrane Retention Signals:
KKXX-like motif in the C-terminus: YTCK
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: nuclear
Reliability: 70.6
COIL: Lupas's algorithm to detect coiled-coil regions
total: 0 residues
Final Results (k = 9/23):
55.6%: extracellular, including cell wall
22.2%: mitochondrial
11.1%: vacuolar
11.1%: nuclear
>> prediction for CG53054-02 is exc (k = 9)

A search of the NOV7a protein against the Geneseq database, a proprietary database that contains sequences published in patents and patent publication, yielded several homologous proteins shown in Table 7D.

TABLE 7D
Geneseq Results for NOV7a

| | | NOV7a | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| AAE34048 | WNT-14 protein - Unidentified, 365 | 2 . . . 357 | 339/356 (95%) | 0.0 |
| | aa. [WO200290992-A2, | 13 . . . 365 | 343/356 (96%) | |
| | 14 NOV. 2002] | | | |
| ABU55894 | Human WNT-14 protein - Homo | 2 . . . 357 | 339/356 (95%) | 0.0 |
| | sapiens, 365 aa. [WO200277204-A2, | 13 . . . 365 | 343/356 (96%) | |
| | 03 OCT. 2002] | | | |
| ABG69638 | Human secreted protein SCEP-18 - | 2 . . . 357 | 311/357 (87%) | 0.0 |
| | Homo sapiens, 366 aa. | 13 . . . 366 | 327/357 (91%) | |
| | [WO200248337-A2, 20 JUN. 2002] | | | |
| AAO18744 | Human NOV8 protein - Homo sapiens, | 25 . . . 357 | 302/334 (90%) | 0.0 |
| | 355 aa. [WO200257450-A2, | 22 . . . 355 | 316/334 (94%) | |
| | 25 JUL. 2002] | | | |
| AAE17305 | Human WNT15 protein, | 22 . . . 356 | 210/338 (62%) | e−124 |
| | sbg389686WNT15a #1 -Homo | 14 . . . 346 | 257/338 (75%) | |
| | sapiens, 704 aa. [WO200198342-A1, | | | |
| | 27 DEC. 2001] | | | |

In a BLAST search of public sequence databases, the NOV7a protein was found to have homology to the proteins shown in the BLASTP data in Table 7E.

TABLE 7E
Public BLASTP Results for NOV7a

| | | NOV7a | Identities/ | |
|---|---|---|---|---|
| Protein | | Residues/ | Similarities for | |
| Accession | | Match | the Matched | Expect |
| Number | Protein/Organism/Length | Residues | Portion | Value |
| O14904 | Wnt-9a protein precursor (Wnt-14) - | 2 . . . 357 | 339/356 (95%) | 0.0 |
| | Homo sapiens (Human), 365 aa. | 13 . . . 365 | 343/356 (96%) | |
| Q8R5M2 | Wnt-9a protein precursor (Wnt-14) - | 2 . . . 357 | 333/356 (93%) | 0.0 |
| | Mus musculus (Mouse), 365 aa. | 13 . . . 365 | 340/356 (94%) | |
| O42280 | Wnt-9a protein precursor (Wnt-14) - | 25 . . . 356 | 283/333 (84%) | e−173 |
| | Gallus gallus (Chicken), 354 aa. | 24 . . . 353 | 310/333 (92%) | |
| Q8C718 | WNT14B - Mus musculus (Mouse), | 8 . . . 356 | 216/354 (61%) | e−125 |
| | 359 aa. | 12 . . . 358 | 264/354 (74%) | |
| O35468 | Wnt-9b protein precursor (Wnt-15) | 8 . . . 356 | 216/354 (61%) | e−125 |
| | (Wnt-14b) - Mus musculus (Mouse), | 12 . . . 358 | 264/354 (74%) | |
| | 359 aa. | | | |

PFam analysis predicts that the NOV7a protein contains the domains shown in the Table 7F.

TABLE 7F
Domain Analysis of NOV7a

| | | Identities/ | |
|---|---|---|---|
| Pfam | | Similarities | Expect |
| Domain | NOV7a Match Region | for the Matched Region | Value |
| wnt | 50 . . . 356 | 129/359 (36%) | 4.6e−103 |
| | | 234/359 (65%) | |

### Example 8

The NOV8 clone was analyzed, and the nucleotide and encoded polypeptide sequences are shown in Table 8A.

TABLE 8A
NOV8 Sequence Analysis

| | | |
|---|---|---|
| NOV8a, CG53473-02 | SEQ ID NO: 101 | 514 bp |
| DNA Sequence | ORF Start: ATG at 37 | ORF Stop: TGA at 400 |

CGCGCGCCCGAACGAAGCCGCGGCCCGGGCACAGCCATGGCCCGGCGGGCGGGGGGCGCTCGGATGTTCGGCA
GCCTCCTGCTCTTCGCCCTGCTCGCTGCCGGCGTCGCCCCGCTCAGCTGGGATCTCCCGGAGCCCCGCAGCCG
AGCCAGCAAGATCCGAGTGCACTCGCGAGGCAACCTCTGGGCCACCGGTCACTTCATGGGCAAGAAGAGTCTG
GAGCCTTCCAGCCCATCCCCATTGGGGACAGCTCCCCACACCTCCCTGAGGGACCAGCGACTGCAGCTGAGTC
ATGATCTGCTCGGAATCCTCCTGCTAAAGAAGGCTCTGGGCGTGAGCCTCAGCCGCCCCGCACCCCAAATCCA
GTACAGGAGGCTGCTGGTACAAATACTGCAGAAATGACACCAATAATGGGGCAGACACAACAGCCTGCCTTAG
ATTGTGCCCACCCAGGGAAGGTGCTGAATGGGACCCTGTTGATGGCCATCAACAGGGTCCCATTCAGCACAGG
CTG

| | | | |
|---|---|---|---|
| NOV8a, CG53473-02 | | | |
| Protein Sequence | SEQ ID NO: 102 | 121 aa | MW at 13251.4kD |

MARRAGGARMFGSLLLFALLAAGVAPLSWDLPEPRSRASKIRVHSRGNLWATGHFMGKKSLEPSSPSPLGTAP
HTSLRDQRLQLSHDLLGILLLKKALGVSLSRPAPQIQYRRLLVQILQK

| | | |
|---|---|---|
| NOV8b, CG53473-01 | SEQ ID NO: 103 | 646 bp |
| DNA Sequence | ORF Start: ATG at 62 | ORF Stop: TGA at 398 |

AGCGCGCCCGAACGAAGCCGCGGCCCGGGCACAGCATGGCCCGCGGCGGGAGGGCGCTCGGATGTTCGGCAGC
CTCCTGCACTTCGCCCTGCTCGCTGCCGGCGTCGTCCCGCTCAGCTGGGATCTCCCGGAGCCCCGCAGCCGAG
CCAGCAAGATCCGAGTGCACTCGCGAGGCAAGCTCTGGGCCATCGGTCACTTCATGGGCAAGAAGAGTCTGGA
GCCTTCCAGCCCATCCCCATTGGGGACAGCTCCCCACACCTCCCTGAGGGACCAGCGACTGCAGCTGAGTCAT
GATCTGCTCGGAATCCTCCTGCTAAAGAAGGCTCTGGGCGTGAGCCTCAGCCGCCCCGCACCCCAAATCCAGT
ACAGGAGGCTGCTGGTACAAATACTGCAGAAATGACACCAATAATGGGGCAGACACAACAGCGTGGCTTAGAT
TGTGCCCACCCAGGGAAGGTGCTGAATGGGACCCTGTTGATGGCCCCATCTGGATGTAAATCCTGAGCTCAAA
TCTCTGTTACTCCATTACTGTGATTTCTGGCTGGGTCACCAGAAATATCGCTGATGCAGACACAGATTATGTT
CCTGCTGTATTTCCTGCTTCCCTGTTGAATTGGTGAATAAAACCTTGCTCTATACATACAAA

| | | | |
|---|---|---|---|
| NOV8b, CG53473-01 | | | |
| Protein Sequence | SEQ ID NO: 104 | 112 aa | MW at 12402.5kD |

MFGSLLHFALLAAGVVPLSWDLPEPRSRASKIRVHSRGKLWAIGHFMGKKSLEPSSPSPLGTAPHTSLRDQRL
QLSHDLLGILLLKKALGVSLSRPAPQIQYRRLLVQILQK

| | | |
|---|---|---|
| NOV8c, CG53473-03 | SEQ ID NO: 105 | 30 bp |
| DNA Sequence | ORF Start: at 1 | ORF Stop: end of sequence |

GGCAAGCTCTGGGCCATCGGTCACTTCATG

| | | | |
|---|---|---|---|
| NOV8c, CG53473-03 | | | |
| Protein Sequence | SEQ ID NO: 106 | 10 aa | MW at 1159.4kD |

GKLWAIGHFM

| | | |
|---|---|---|
| | SEQ ID NO: 107 | 514 bp |
| NOV8d, SNP13376396 of | ORF Start: ATG at 37 | ORF Stop: TGA at 400 |
| CG53473-02, DNA Sequence | SNP Pos: 190 | SNP Change: A to G |

CGCGCGCCCGAACGAAGCCGCGGCCCGGGCACAGCCATGGCCCGGCGGGCGGGGGGCGCTCGGATGTTCGGCA
GCCTCCTGCTCTTCGCCCTGCTCGCTGCCGGCGTCGCCCCGCTCAGCTGGGATCTCCCGGAGCCCCGCAGCCG
AGCCAGCAAGATCCGAGTGCACTCGCGAGGCAACCTCTGGGCCGCCGGTCACTTCATGGGCAAGAAGAGTCTG
GAGCCTTCCAGCCCATCCCCATTGGGGACAGCTCCCCACACCTCCCTGAGGGACCAGCGACTGCAGCTGAGTC
ATGATCTGCTCGGAATCCTCCTGCTAAAGAAGGCTCTGGGCGTGAGCCTCAGCCGCCCCGCACCCCAAATCCA
GTACAGGAGGCTGCTGGTACAAATACTGCAGAAATGACACCAATAATGGGGCAGACACAACAGCGTGGCTTAG
ATTGTGCCCACCCAGGGAAGGTGCTGAATGGGACCCTGTTGATGGCCATCAACAGGGTCCCATTCAGCACAGG
CTG

| | | | |
|---|---|---|---|
| NOV8d, SNP13376396 of | SEQ ID NO: 108 | | MW at 13221.4kD |
| CG53473-02, Protein Sequence | SNP Pos: 52 | 121 aa | SNP Change: Thr to Ala |

MARRAGGARMFGSLLLFALLAAGVAPLSWDLPEPRSRASKIRVHSRGNLWAAGHFMGKKSLEPSSPSPLGTAP
HTSLRDQRLQLSHDLLGILLLKKALGVSLSRPAPQIQYRRLLVQILQK

| | | |
|---|---|---|
| | SEQ ID NO: 109 | 514 bp |
| NOV8e, SNP13376395 of | ORF Start: ATG at 37 | ORF Stop: TGA at 400 |
| CG53473-02, DNA Sequence | SNP Pos: 253 | SNP Change: C to A |

CGCGCGCCCGAACGAAGCCGCGGCCCGGGCACAGCCATGGCCCGGCGGGCGGGGGGCGCTCGGATGTTCGGCA
GCCTCCTGCTCTTCGCCCTGCTCGCTGCCGGCGTCGCCCCGCTCAGCTGGGATCTCCCGGAGCCCCGCAGCCG
AGCCAGCAAGATCCGAGTGCACTCGCGAGGCAACCTCTGGGCCACCGGTCACTTCATGGGCAAGAAGAGTCTG
GAGCCTTCCAGCCCATCCCCATTGGGGACAGCTACCCACACCTCCCTGAGGGACCAGCGACTGCAGCTGAGTC
ATGATCTGCTCGGAATCCTCCTGCTAAAGAAGGCTCTGGGCGTGAGCCTCAGCCGCCCCGCACCCCAAATCCA
GTACAGGAGGCTGCTGGTACAAATACTGCAGAAATGACACCAATAATGGGGCAGACACAACAGCGTGGCTTAG
ATTGTGCCCACCCAGGGAAGGTGCTGAATGGGACCCTGTTGATGGCCATCAACAGGGTCCCATTCAGCACAGG
CTG

| | | | |
|---|---|---|---|
| NOV8e, SNP13376395 of | SEQ ID NO: 110 | | MW at 13255.4kD |
| CG53473-02, Protein Sequence | SNP Pos: 73 | 121 aa | SNP Change: Pro to Thr |

MARRAGGARMFGSLLLFALLAAGVAPLSWDLPEPRSRASKIRVHSRGNLWATGHFMGKKSLEPSSPSPLGTAT
HTSLRDQRLQLSHDLLGILLLKKALGVSLSRPAPQIQYRRLLVQILQK

| | | |
|---|---|---|
| | SEQ ID NO: 111 | 514 bp |
| NOV8f, SNP13376394 of | ORF Start: ATG at 37 | ORF Stop: TAA at 400 |
| CG53473-02, DNA Sequence | SNP Pos: 401 | SNP Change: G to A |

CGCGCGCCCGAACGAAGCCGCGGCCCGGGCACAGCCATGGCCCGGCGGGCGGGGGGCGCTCGGATGTTCGGCA
GCCTCCTGCTCTTCGCCCTGCTCGCTGCCGGCGTCGCCCCGCTCAGCTGGGATCTCCCGGAGCCCCGCAGCCG
AGCCAGCAAGATCCGAGTGCACTCGCGAGGCAACCTCTGGGCCACCGGTCACTTCATGGGCAAGAAGAGTCTG
GAGCCTTCCAGCCCATCCCCATTGGGGACAGCTCCCCACACCTCCCTGAGGGACCAGCGACTGCAGCTGAGTC
ATGATCTGCTCGGAATCCTCCTGCTAAAGAAGGCTCTGGGCGTGAGCCTCAGCCGCCCCGCACCCCAAATCCA
GTACAGGAGGCTGCTGGTACAAATACTGCAGAAATAACACCAATAATGGGGCAGACACAACAGCGTGGCTTAG
ATTGTGCCCACCCAGGGAAGGTGCTGAATGGGACCCTGTTGATCGCCATCAACAGGGTCCCATTCAGCACAGG
CTG

| | | | |
|---|---|---|---|
| NOV8f, SNP13376394 of | | | MW at 13251.4kD |
| CG53473-02, Protein Sequence | SEQ ID NO: 112 | 112 aa | SNP change: no change |

MARRAGGARMFGSLLLFALLAAGVAPLSWDLPEPRSRASKIRVHSRGNLWATGHFMGKKSLEPSSPSPLGTAP
HTSLRDQRLQLSHDLLGILLLKKALGVSLSRPAPQIQYRRLLVQILQK

A ClustalW comparison of the above protein sequences yields the following sequence alignment shown in Table 8B.

TABLE 8B
Comparison of the NOV8 protein sequences.

| | |
|---|---|
| NOV8a | MARRAGGARMFGSLLLFALLAAGVAPLSWDLPEPRSRASKIRVHSRGNLWATGHFMGKKS |
| NOV8b | ---------MFGSLLHFALLAAGVVPLSWDLPEPRSRASKIRVHSRGKLWAIGHFMGKKS |
| NOV8c | ----------------------------------------------GKLWAIGHFM---- |
| NOV8a | LEPSSPSPLGTAPHTSLRDQRLQLSHDLLGILLLKKALGVSLSRPAPQIQYRRLLVQILQ |
| NOV8b | LEPSSPSPLGTAPHTSLRDQRLQLSHDLLGILLLKKALGVSLSRPAPQIQYRRLLVQILQ |
| NOV8c | ------------------------------------------------------------ |
| NOV8a | K |
| NOV8b | K |
| NOV8c | - |
| NOV8a | (SEQ ID NO: 102) |
| NOV8b | (SEQ ID NO: 104) |
| NOV8c | (SEQ ID NO: 106) |

Further analysis of the NOV8a protein yielded the following properties shown in Table 8C.

TABLE 8C
Protein Sequence Properties NOV8a

| SignalP | | | | |
|---|---|---|---|---|
| analysis: | Cleavage site between residues 27 and 28 | | | |
| PSORT II | PSG: a new signal peptide prediction method | | | |
| analysis: | N-region: length 9; pos. chg 3; neg. chg 0 | | | |
| | H-region: length 20; peak value 10.93 | | | |
| | PSG score: 6.53 | | | |
| | GvH: von Heijne's method for signal seq. recognition | | | |
| | GvH score (threshold: −2.1): 2.23 | | | |
| | possible cleavage site: between 26 and 27 | | | |
| | >>> Seems to have a cleavable signal peptide (1 to 26) | | | |
| | ALOM: Klein et al's method for TM region allocation | | | |
| | Init position for calculation: 27 | | | |
| | Tentative number of TMS(s) for the threshold 0.5: 0 | | | |
| | number of TMS(s) . . . fixed | | | |
| | PERIPHERAL | Likelihood = 1.85 (at 87) | | |
| | ALOM score: 1.85 (number of TMSs: 0) | | | |
| | MTOP: Prediction of membrane topology (Hartmann et al.) | | | |
| | Center position for calculation: 13 | | | |
| | Charge difference: −1.5 C(2.5)-N(4.0) | | | |
| | N >= C: N-terminal side will be inside | | | |
| | MITDISC: discrimination of mitochondrial targeting seq | | | |
| | R content: | 3 | Hyd Moment(75): | 12.45 |
| | Hyd Moment(95): | 10.60 | G content: | 4 |
| | D/E content: | 1 | S/T content: | 2 |

Score: −2.16
Gavel: prediction of cleavage sites for mitochondrial preseq
R-2 motif at 19 ARM|FG
NUCDISC: discrimination of nuclear localization signals
pat4: none
pat7: none
bipartite: none
content of basic residues: 14.9%
NLS Score: −0.47
KDEL: ER retention motif in the C-terminus: none
ER Membrane Retention Signals:
XXRR-like motif in the N-terminus: ARRA
none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: nuclear
Reliability: 89
COIL: Lupas's algorithm to detect coiled-coil regions
total: 0 residues
Final Results (k = 9/23):
44.4%: extracellular, including cell wall
33.3%: mitochondrial
22.2%: nuclear
>> prediction for CG53473-02 is exc (k = 9)

A search of the NOV8a protein against the Geneseq database, a proprietary database that contains sequences published in patents and patent publication, yielded several homologous proteins shown in Table 8D.

TABLE 8D
Geneseq Results for NOV8a

| | | NOV8a | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| AAE17605 | Human extracellular messenger | 1 . . . 121 | 121/121 (100%) | 1e−63 |
| | (XMES)-7 protein - Homo sapiens, | 1 . . . 121 | 121/121 (100%) | |
| | 121 aa. [WO200194587-A2, | | | |
| | 13 DEC 2001] | | | |
| ABP51992 | NOVNEUR homologous amino acid | 1 . . . 121 | 114/121 (94%) | 3e−58 |
| | sequence SEQ ID NO: 29 - Homo | 1 . . . 121 | 114/121 (94%) | |
| | sapiens, 121 aa. [US2002068279-A1, | | | |
| | 06 JUN. 2002] | | | |
| ABP51987 | NOVNEUR homologous amino acid | 4 . . . 121 | 112/118 (94%) | 7e−58 |
| | sequence SEQ ID NO: 24 - Homo | 1 . . . 118 | 112/118 (94%) | |
| | sapiens, 118 aa. [US2002068279-A1, | | | |
| | 06 JUN. 2002] | | | |
| ABP51989 | NOVNEUR homologous amino acid | 4 . . . 121 | 111/118 (94%) | 1e−56 |
| | sequence SEQ ID NO: 26 - Homo | 1 . . . 118 | 111/118 (94%) | |
| | sapiens, 118 aa. [US2002068279-A1, | | | |
| | 06 JUN. 2002] | | | |
| ABP51990 | NOVNEUR homologous amino acid | 10 . . . 121 | 108/112 (96%) | 7e−56 |
| | sequence SEQ ID NO: 27 - Homo | 1 . . . 112 | 108/112 (96%) | |
| | sapiens, 112 aa. [US2002068279-A1, | | | |
| | 06 JUN. 2002] | | | |

In a BLAST search of public sequence databases, the NOV8a protein was found to have homology to the proteins shown in the BLASTP data in Table 8E.

TABLE 8E
Public BLASTP Results for NOV8a

| | | NOV8a | Identities/ | |
|---|---|---|---|---|
| Protein | | Residues/ | Similarities for | |
| Accession | | Match | the Matched | Expect |
| Number | Protein/Organism/Length | Residues | Portion | Value |
| P08949 | Neuromedin B-32 precursor | 1 . . . 121 | 120/121 (99%) | 2e−62 |
| | [Contains: Neuromedin B] - Homo | 1 . . . 121 | 120/121 (99%) | |
| | sapiens (Human), 121 aa. | | | |
| Q9CR53 | Neuromedin B-32 precursor | 1 . . . 121 | 89/121 (73%) | 2e−43 |
| | [Contains: Neuromedin B] - Mus | 1 . . . 121 | 99/121 (81%) | |
| | musculus (Mouse), 121 aa. | | | |
| A37178 | neuromedin B precursor - rat, 117 aa. | 1 . . . 115 | 84/115 (73%) | 2e−41 |
| | | 1 . . . 115 | 94/115 (81%) | |
| A28945 | neuromedin B precursor - human, 76 | 1 . . . 73 | 69/73 (94%) | 5e−33 |
| | aa. | 1 . . . 73 | 69/73 (94%) | |
| P01297 | Neuromedin B-32 [Contains: | 25 . . . 56 | 30/32 (93%) | 2e−11 |
| | Neuromedin B] - Sus scrofa (Pig), 32 | 1 . . . 32 | 30/32 (93%) | |
| | aa. | | | |

PFam analysis predicts that the NOV8a protein contains the domains shown in the Table 8F.

TABLE 8F
Domain Analysis of NOV8a

| | | Identities/ | |
|---|---|---|---|
| | NOV8a Match | Similarities | Expect |
| Pfam Domain | Region | for the Matched Region | Value |
| Bombesin | 47 . . . 56 | 8/10 (80%) | 0.26 |
| | | 10/10 (100%) | |

### Example 9

The NOV9 clone was analyzed, and the nucleotide and encoded polypeptide sequences are shown in Table 9A.

TABLE 9A
NOV9 Sequence Analysis

| | | |
|---|---|---|
| NOV9a, CG55184-03 | SEQ ID NO: 113 | 614 bp |
| DNA Sequence | ORF Start: ATG at 4 | ORF Stop: TAG at 607 |

ACCATGGGCTCCGGGCGCCGGGCGCTGTCCGCGGTGCCGGCCGTGCTGCTGGTCCTCACGCTGCCGGGGCTGC
CCGTCTGGGCACAGAACGACACGGAGCCCATCGTGCTGGAGGGCAAGTGTCTGGTGGTGTGCGACTCGAACCC
GGCCACGGACTCCAAGGGCTCCTCTTCCTCCCCGCTGGGGATATCGGTCCGGGCGGCCAACTCCAAGGTCGCC
TTCTCGGCGGTGCGGAGCACCAACCACGAGCCATCCGAGATGAGCAACAAGACGCGCATCATTTACTTCGATC
AGATCCTGGTGAATGTGGGTAATTTTTTCACATTGGAGTCTGTCTTTGTAGCACCAAGAAAAGGAATTTACAG
TTTCAGTTTTCACGTGATTAAAGTCTACCAGAGCCAAACTATCCAGGTTAACTTGATGTTAAATGGAAAACCA
GTAATATCTGCCTTTGCGGGGGACAAAGATGTTACTCGTGAAGCTGCCACGAATGGTGTCCTGCTCTACCTAG
ATAAAGAGGATAAGGTTTACCTAAAACTGGAGAAAGGTAATTTGGTTGGAGGCTGGCAGTATTCCACGTTTTC
TGGCTTTCTGGTGTTCCCCCTATAGGATTC

| | | | |
|---|---|---|---|
| NOV9a, CG55184-03 | | | |
| Protein Sequence | SEQ ID NO: 114 | 201 aa | MW at 21807.9kD |

MGSGRRALSAVPAVLLVLTLPGLPVWAQNDTEPIVLEGKCLVVCDSNPATDSKGSSSSPLGISVRAANSKVAF
SAVRSTNHEPSEMSNKTRIIYFDQILVNVGNFFTLESVFVAPRKGIYSFSFHVIKVYQSQTIQVNLMLNGKPV
ISAFAGDKDVTREAATNGVLLYLDKEDKVYLKLEKGNLVGGWQYSTFSGFLVFPL

| | | |
|---|---|---|
| NOV9b, CG55184-01 | SEQ ID NO: 115 | 614 bp |
| DNA Sequence | ORF Start: ATG at 4 | ORF Stop: TAG at 607 |

ACCATGGGCTCCGGGCGCCGGGCGCTGTCCGCGGTGCCGGCCGTGCTGCTGGTCCTCACGCTGCCGGGGCTGC
CCGTCTGGGCACAGAACGACACGGAGCCCATCGTGCTGGAGGGCAAGTGTCTGGTGGTGTGCGACTCGAACCC
GGCCACGGACTCCAAGGGCTCCTCTTCCTCCCCGCTGGGGATATCGGTCCGGGCGGCCAACTCCAAGGTCGCC
TTCTCGGCGGTGCGGAGCACCAACCACGAGCCATCCGAGATGAGCAACAAGACGCGCATCATTTACTTCGATC
AGATCCTGGTGAATGTGGGTAATTTTTTCACATTGGAGTCTGTCTTTGTAGCACCAAGAAAAGGAATTTACAG
TTTCAGTTTTCACGTGATTAAAGTCTACCAGAGCCAAACTATCCAGGTTAACTTGATGTTAAATGGAAAACCA
GTAATATCTGCCTTTGCGGGGGACAAAGATGTTACTCGTGAAGCTGCCACGAATGGTGTCCTGCTCTACCTAG
ATAAAGAGGATAAGGTTTACCTAAAACTGGAGAAAGGTAATTTGGTTGGAGGCTGGCAGTATTCCACGTTTTC
TGGCTTTCTGGTGTTCCCCCTATAGGATTC

| | | | |
|---|---|---|---|
| NOV9b, CG55184-01 | | | |
| Protein Sequence | SEQ ID NO: 116 | 201 aa | MW at 21807.9kD |

MGSGRRALSAVPAVLLVLTLPGLPVWAQNDTEPIVLEGKCLVVCDSNPATDSKGSSSSPLGISVRAANSKVAF
SAVRSTNHEPSEMSNKTRIIYFDQILVNVGNFFTLESVFVAPRKGIYSFSFHVIKVYQSQTIQVNLMLNGKPV
ISAFAGDKDVTREAATNGVLLYLDKEDKVYLKLEKGNLVGGWQYSTFSGFLVFPL

| | | |
|---|---|---|
| NOV9c, CG55184-02 | SEQ ID NO: 117 | 522 bp |
| DNA Sequence | ORF Start: at 1 | ORF Stop: end of sequence |

CAGAACGACACGGAGCCCATTGTGCTGGAGGGCAAGTGTCTGGTGGTGTGCGACTCGAACCCGGCCACGGACT
CCAAGGGCTCCTCTTCCTCCCCGCTGGGGATATCGGTCCGGGCGGCCAACTCCAAGGTCGCCTTCTCGGCGGT
GCGGAGCACCAACCACGAGCCATCCGAGATGAGCAACAAGACGCGCATCATTTACTTCGATCAGATCCTGGTG
AATGTGGGTAATTTTTTCACATTGGAGTCTGTCTTTGTAGCACCAAGAAAAGGAATTTACAGTTTCAGTTTTC
ACGTGATTAAAGTCTACCAGAGCCAAACTATCCAGGTTAACTTGATGTTAAATGGAAAACCAGTAATATCTGC
CTTTGCGGGGGACAAAGATGTTACTCGTGAAGCTGCCACGAATGGTGTCCTGCTCTACCTAGATAAAGAGGAT
AAGGTTTACCTAAAACTGGAGAAAGGTAATTTGGTTGGAGGCTGGCAGTATTCCACGTTTTCTGGCTTTCTGG
TGTTCCCCCTA

| | | | |
|---|---|---|---|
| NOV9c, CG55184-02 | | | |
| Protein Sequence | SEQ ID NO: 118 | 174 aa | MW at 19080.6kD |

QNDTEPIVLEGKCLVVCDSNPATDSKGSSSSPLGISVRAANSKVAFSAVRSTNHEPSEMSNKTRIIYFDQILV
NVGNFFTLESVFVAPRKGIYSFSFHVIKVYQSQTIQVNLMLNGKPVISAFAGDKDVTREAATNGVLLYLDKED
KVYLKLEKGNLVGGWQYSTFSGFLVFPL

| | | |
|---|---|---|
| NOV9d, CG55184-04 | SEQ ID NO: 119 | 148 bp |
| DNA Sequence | ORF Start: at 1 | ORF Stop: end of sequence |

GCGGCCAACTCCAAGGTCGCCTTCTCGGCGGTGCGGAGCACCAACCAC

| | | | |
|---|---|---|---|
| NOV9d, CG55184-04 | | | |
| Protein Sequence | SEQ ID NO: 120 | 16 aa | MW at 1659.8kD |

AANSKVAFSAVRSTNH

| | | |
|---|---|---|
| NOV9e, CG55184-05 | SEQ ID NO: 121 | 45 bp |
| DNA Sequence | ORF Start: at 1 | ORF Stop: end of sequence |

GCCAACTCCAAGGTCGCCTTCTCGGCGGTGCGGAGCACCAACCAC

| | | | |
|---|---|---|---|
| NOV9e, CG55184-05 | | | |
| Protein Sequence | SEQ ID NO: 122 | 15 aa | MW at 1588.7kD |

ANSKVAFSAVRSTNH

A ClustalW comparison of the above protein sequences yields the following sequence alignment shown in Table 9B.

TABLE 9B
Comparison of the NOV9 protein sequences.

| | |
|---|---|
| NOV9a | MGSGRRALSAVPAVLLVLTLPGLPVWAQNDTEPIVLEGKCLVVCDSNPATDSKGSSSSPL |
| NOV9b | MGSGRRALSAVPAVLLVLTLPGLPVWAQNDTEPIVLEGKCLVVCDSNPATDSKGSSSSPL |
| NOV9c | ---------------------------QNDTEPIVLEGKCLVVCDSNPATDSKGSSSSPL |
| NOV9d | ------------------------------------------------------------ |
| NOV9e | ------------------------------------------------------------ |
| NOV9a | GISVRAANSKVAFSAVRSTNHEPSEMSNKTRIIYFDQILVNVGNFFTLESVFVAPRKGIY |
| NOV9b | GISVRAANSKVAFSAVRSTNHEPSEMSNKTRIIYFDQILVNVGNFFTLESVFVAPRKGIY |
| NOV9c | GISVRAANSKVAFSAVRSTNHEPSEMSNKTRIIYFDQILVNVGNFFTLESVFVAPRKGIY |
| NOV9d | -----AANSKVAFSAVRSTNH--------------------------------------- |
| NOV9e | ------ANSKVAFSAVRSTNH--------------------------------------- |
| NOV9a | SFSFHVIKVYQSQTIQVNLMLNGKPVISAFAGDKDVTREAATNGVLLYLDKEDKVYLKLE |
| NOV9b | SFSFHVIKVYQSQTIQVNLMLNGKPVISAFAGDKDVTREAATNGVLLYLDKEDKVYLKLE |
| NOV9c | SFSFHVIKVYQSQTIQVNLMLNGKPVISAFAGDKDVTREAATNGVLLYLDKEDKNYLKLE |
| NOV9d | ------------------------------------------------------------ |
| NOV9e | ------------------------------------------------------------ |
| NOV9a | KGNLVGGWQYSTFSGFLVFPL |
| NOV9b | KGNLVGGWQYSTFSGFLVFPL |
| NOV9c | KGNLVGGWQYSTFSGFLVFPL |
| NOV9d | --------------------- |
| NOV9e | --------------------- |
| NOV9a | (SEQ ID NO: 114) |
| NOV9b | (SEQ ID NO: 116) |
| NOV9c | (SEQ ID NO: 118) |
| NOV9d | (SEQ ID NO: 120) |
| NOV9e | (SEQ ID NO: 122) |

Further analysis of the NOV9a protein yielded the following properties shown in Table 9C.

TABLE 9C
Protein Sequence Properties NOV9a

| SignalP | Cleavage site between residues 28 and 29 | | | |
|---|---|---|---|---|
| analysis: | | | | |
| PSORT II | PSG: a new signal peptide prediction method | | | |
| analysis: | N-region: length 6; pos. chg 2; neg. chg 0 | | | |
| | H-region: length 23; peak value 10.04 | | | |
| | PSG score: 5.64 | | | |
| | GvH: von Heijne's method for signal seq. recognition | | | |
| | GvH score (threshold: −2.1): 0.95 | | | |
| | possible cleavage site: between 27 and 28 | | | |
| | >>> Seems to have a cleavable signal peptide (1 to 27) | | | |
| | ALOM: Klein et al's method for TM region allocation | | | |
| | Init position for calculation: 28 | | | |
| | Tentative number of TMS(s) for the threshold 0.5: 1 | | | |
| | Number of TMS(s) for threshold 0.5: 0 | | | |
| | PERIPHERAL | Likelihood = 5.67 (at 60) | | |
| | ALOM score: 0.10 (number of TMSs: 0) | | | |
| | MTOP: Prediction of membrane topology (Hartmann et al.) | | | |
| | Center position for calculation: 13 | | | |
| | Charge difference: −5.0 C(−2.0)-N(3.0) | | | |
| | N >= C: N-terminal side will be inside | | | |
| | MITDISC: discrimination of mitochondrial targeting seq | | | |
| | R content: | 2 | Hyd Moment(75): | 11.01 |
| | Hyd Moment(95): | 9.83 | G content: | 3 |
| | D/E content: | 1 | S/T content: | 3 |

Score: −2.58
Gavel: prediction of cleavage sites for mitochondrial preseq
R-2 motif at 16 RRA|LS
NUCDISC: discrimination of nuclear localization signals
pat4: none
pat7: none
bipartite: none
content of basic residues: 9.5%
NLS Score: −0.47
KDEL: ER retention motif in the C-terminus: none
ER Membrane Retention Signals:
XXRR-like motif in the N-terminus: GSGR
none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 94.1
COIL: Lupas's algorithm to detect coiled-coil regions
total: 0 residues
Final Results (k = 9/23):
33.3%: extracellular, including cell wall
33.3%: mitochondrial
22.2%: endoplasmic reticulum
11.1%: Golgi
>> prediction for CG55184-03 is exc (k = 9)

A search of the NOV9a protein against the Geneseq database, a proprietary database that contains sequences published in patents and patent publication, yielded several homologous proteins shown in Table 9D.

TABLE 9D
Geneseq Results for NOV9a

| | | NOV9a | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| AAE16346 | Human cerebellin-like protein, | 1 . . . 201 | 201/201 (100%) | e−111 |
| | POLY10 - Homo sapiens, 201 aa. | 1 . . . 201 | 201/201 (100%) | |
| | [WO200185767-A2, 15 NOV. 2001] | | | |
| ABB84924 | Human PRO1382 protein sequence | 1 . . . 201 | 201/201 (100%) | e−111 |
| | SEQ ID NO: 216 - Homo sapiens, 201 | 1 . . . 201 | 201/201 (100%) | |
| | aa. [WO200200690-A2, | | | |
| | 03 JAN. 2002] | | | |
| ABB95530 | Human angiogenesis related protein | 1 . . . 201 | 201/201 (100%) | e−111 |
| | PRO1382 SEQ ID NO: 216 - Homo | 1 . . . 201 | 201/201 (100%) | |
| | sapiens, 201 aa. [WO200208284-A2, | | | |
| | 31 JAN. 2002] | | | |
| AAO15422 | Human genset metabolic gene | 1 . . . 201 | 201/201 (100%) | e−111 |
| | (GMG-8) protein - Homo sapiens, 201 | 1 . . . 201 | 201/201 (100%) | |
| | aa. [WO200255694-A2, | | | |
| | 18 JUL. 2002] | | | |
| AAB66151 | Protein of the invention #63 - | 1 . . . 201 | 201/201 (100%) | e−111 |
| | Unidentified, 201 aa. | 1 . . . 201 | 201/201 (100%) | |
| | [WO200078961-A1, 28 DEC. 2000] | | | |

In a BLAST search of public sequence databases, the NOV9a protein was found to have homology to the proteins shown in the BLASTP data in Table 9E.

TABLE 9E
Public BLASTP Results for NOV9a

| | | NOV9a | | |
|---|---|---|---|---|
| Protein | | Residues/ | Identities/ | |
| Accession | | Match | Similarities for the | Expect |
| Number | Protein/Organism/Length | Residues | Matched Portion | Value |
| Q9NTU7 | Cerebellin-like glycoprotein 1 | 1 . . . 201 | 201/201 (100%) | e−111 |
| | precursor - Homo sapiens (Human), | 1 . . . 201 | 201/201 (100%) | |
| | 201 aa. | | | |
| Q8BME9 | CEREBELLIN-like glycoprotein | 1 . . . 201 | 193/201 (96%) | e−105 |
| | precursor - Mus musculus (Mouse), | 1 . . . 198 | 195/201 (96%) | |
| | 198 aa. | | | |
| Q8BMF0 | CEREBELLIN-like glycoprotein | 1 . . . 201 | 192/201 (95%) | e−104 |
| | precursor - Mus musculus (Mouse), | 1 . . . 198 | 194/201 (95%) | |
| | 198 aa. | | | |
| Q8BGU2 | Cerebellin 2 precursor protein - Mus | 7 . . . 201 | 145/196 (73%) | 2e−76 |
| | musculus (Mouse), 224 aa. | 31 . . . 224 | 170/196 (85%) | |
| P98087 | Cerebellin-like glycoprotein 1 - | 7 . . . 201 | 144/196 (73%) | 6e−76 |
| | Rattus norvegicus (Rat), 224 aa. | 31 . . . 224 | 169/196 (85%) | |

PFam analysis predicts that the NOV9a protein contains the domains shown in the Table 9F.

TABLE 9F
Domain Analysis of NOV9a

| | | Identities/ | |
|---|---|---|---|
| Pfam | | Similarities | Expect |
| Domain | NOV9a Match Region | for the Matched Region | Value |
| C1q | 72 . . . 198 | 48/137 (35%) | 1.4e−48 |
| | | 113/137 (82%) | |

### Example 10

The NOV10 clone was analyzed, and the nucleotide and encoded polypeptide sequences are shown in Table 10A.

TABLE 10A
NOV10 Sequence Analysis

| | | |
|---|---|---|
| NOV10a, CG55274-05 | SEQ ID NO: 123 | 274 bp |
| DNA Sequence | ORF Start: ATG at 7 | ORF Stop: TAG at 265 |

ACCACCATGGCACTGCAGGCTGAATTCGACAAGGCTGCAGAAGACGTGAGGAAGCTGCCAACAAGACCAGCAG
ATAATAAAGAACTGAAAAAACTCGATGGACTTTACAAACAAGCTATAATTGGAGACATTAATATTGAGTATCT
GGGAATGCTGGATTTAAAGGGCAAGGCCAAATGCGCAGCATGGACCCTCCAAAAAAGGTTGTCAAAGGAAGAT
GCAACGAGTGTCTCTATTTCTAAGGCAAAAGAGCCGATAGAAAAATAGGACATTT

| | | | |
|---|---|---|---|
| NOV10a, CG55274-05 | | | |
| Protein Sequence | SEQ ID NO: 124 | 86 aa | MW at 9590.0kD |

MALQAEFDKAAEDVRKLPTRPADNKELKKLDGLYKQAIIGDINIEYLGMLDLKGKAKCAAWTLQKRLSKEDAT
SVSISKAKEPIEK

| | | |
|---|---|---|
| NOV10b, CG55274-01 | SEQ ID NO: 125 | 280 bp |
| DNA Sequence | ORF Start: ATG at 7 | ORF Stop: TAG at 265 |

ACCACCATGGCACTGCAGGCTGAATTCGACAAGGCTGCAGAAGACGTGAGGAAGCTGCCAACAAGACCAGCAG
ATAATAAAGAACTGAAAAAACTCGATGGACTTTACAAACAAGCTATAATTGGAGACATTAATATTGAGTATCT
GGGAATGCTGGACTTTAAGGGCAAGGCCAAATGCGCAGCATGGACCCTCCAAAAAAGGTTGTCAAAGGAAGAT
GCAACGAGTGTCTCTATTTCTAAGGCAAAAGAGCCGATAGAAAAATAGGACATTTAGAATA

| | | | |
|---|---|---|---|
| NOV10b, CG55274-01 | | | |
| Protein Sequence | SEQ ID NO: 126 | 86 aa | MW at 9624.1kD |

MALQAEFDKAAEDVRKLPTRPADNKELKKLDGLYKQAIIGDINIEYLGMLDFKGKAKCAAWTLQKRLSKEDAT
SVSISKAKEPIEK

| | | |
|---|---|---|
| NOV10c, CG55274-02 | SEQ ID NO: 127 | 289 bp |
| DNA Sequence | ORF Start: ATG at 17 | ORF Stop: TAG at 272 |

TGCGGCCGCCACCACCATGGCACTGCAGGCTGATCGAGACAAGGCTGCAGAAGACGTGAGGAAGCTGCCAACA
AGACCAGATGAGAAAGAACTGAAAAAACTCGATGGACTTTACAAACAAGCTATAATTGGAGACATTAATATTG
AGTATCTGGGAATGCTGGATTTAAAGGGCAAGGCCAAATGCGCAGCATGGACCCTCCAAAAAAGGTTGTCAAA
GGAAGATGCAACGAGTGTCTCTATTTCTAAGGCAAAACAGCCGATAGAAAAATAGGACATTTAGAATACA

| | | | |
|---|---|---|---|
| NOV10c, CG55274-02 | | | |
| Protein Sequence | SEQ ID NO: 128 | 85 aa | MW at 9528.9kD |

MALQADRDKAAEDVRKLPTRPDEKELKKLDGLYKQAIIGDINIEYLGMLDLKGKAKCAAWTLQKRLSKEDATS
VSISKAKEPIEK

| | | |
|---|---|---|
| NOV10d, CG55274-03 | SEQ ID NO: 129 | 60 bp |
| DNA Sequence | ORF: Start a 1 | ORF Stop: end of sequence |

CAAGCTATAATTGGAGACATTAATATTGAGTATCTGGGAATGCTGGATTTAAAGGGCAAG

| | | | |
|---|---|---|---|
| NOV10d, CG55274-03 | | | |
| Protein Sequence | SEQ ID NO: 130 | 20 aa | MW at 2204.6kD |

QAIIGDINIEYLGMLDLKGK

| | | |
|---|---|---|
| NOV10e, CG55274-04 | SEQ ID NO: 131 | 54 bp |
| DNA Sequence | ORF Start: at 1 | ORF Stop: end of sequence |

CAAGCTATAATTGGAGACATTAATATTGAGTATCTGGGAATGCTGGACTTTAAG

| | | | |
|---|---|---|---|
| NOV10e, CG55274-04 | | | |
| Protein Sequence | SEQ ID NO: 132 | 18 aa | MW at 2053.4kD |

QAIIGDINIEYLGMLDFK

A ClustalW comparison of the above protein sequences yields the following sequence alignment shown in Table 10B.

TABLE 10B
Comparison of the NOV10 protein sequences.

| | |
|---|---|
| NOV10a | MALQAEFDKAAEDVRKLPTRPADNKELKKLDGLYKQAIIGDINIEYLGMLDLKGKAKCAA |
| NOV10b | MALQAEFDKAAEDVRKLPTRPADNKELKKLDGLYKQAIIGDINIEYLGMLDFKGKAKCAA |
| NOV10c | MALQADRDKAAEDVRKLPTRPDE-KELKKLDGLYKQAIIGDINIEYLGMLDLKGKAKCAA |
| NOV10d | -----------------------------------QAIIGDINIEYLGMLDLKGK----- |
| NOV10e | -----------------------------------QAIIGDINIEYLGMLDFK------- |
| NOV10a | WTLQKRLSKEDATSVSISKAKEPIEK |
| NOV10b | WTLQKRLSKEDATSVSTSKAKEPIEK |
| NOV10c | WTLQKRLSKEDATSVSISKAKEPIEK |
| NOV10d | -------------------------- |
| NOV10e | -------------------------- |
| NOV10a | (SEQ ID NO: 124) |
| NOV10b | (SEQ ID NO: 126) |
| NOV10c | (SEQ ID NO: 128) |
| NOV10d | (SEQ ID NO: 130) |
| NOV10e | (SEQ ID NO: 132) |

Further analysis of the NOV10a protein yielded the following properties shown in Table 10C.

TABLE 10C
Protein Sequence Properties NOV10a

| SignalP | No Known Signal Sequence Predicted | | | |
|---|---|---|---|---|
| analysis: | | | | |
| PSORT II | PSG: a new signal peptide prediction method | | | |
| analysis: | N-region: length 9; pos. chg 1; neg. chg 2 | | | |
| | H-region: length 2; peak value 0.00 | | | |
| | PSG score: −4.40 | | | |
| | GvH: von Heijne's method for signal seq. recognition | | | |
| | GvH score (threshold: −2.1): −10.68 | | | |
| | possible cleavage site: between 58 and 59 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 0 | | | |
| | number of TMS(s) . . . fixed | | | |
| | PERIPHERAL | Likelihood = 7.11 (at 36) | | |
| | ALOM score: 7.11 (number of TMSs: 0) | | | |
| | MITDISC: discrimination of mitochondrial targeting seq | | | |
| | R content: | 0 | Hyd Moment(75): | 3.71 |
| | Hyd Moment(95): | 2.95 | G content: | 0 |
| | D/E content: | 2 | S/T content: | 0 |

```
Score: −7.75
Gavel: prediction of cleavage sites for mitochondrial preseq
cleavage site motif not found
NUCDISC: discrimination of nuclear localization signals
pat4: none
pat7: none
bipartite: none
content of basic residues: 19.8%
NLS Score: −0.47
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: nuclear
Reliability: 76.7
COIL: Lupas's algorithm to detect coiled-coil regions
total: 0 residues
Final Results (k = 9/23):
82.6%: nuclear
  4.3%: cytoskeletal
  4.3%: mitochondrial
  4.3%: cytoplasmic
  4.3%: peroxisomal
>> prediction for CG55274-05 is nuc (k = 23)
```

A search of the NOV10a protein against the Geneseq database, a proprietary database that contains sequences published in patents and patent publication, yielded several homologous proteins shown in Table 10D.

TABLE 10D
Geneseq Results for NOV10a

| | | NOV10a | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| AAB81814 | Human endozepine-like ENDO5 SEQ | 1 . . . 86 | 85/86 (98%) | 1e−42 |
| | ID NO: 10 - Homo sapiens, 86 aa. | 1 . . . 86 | 85/86 (98%) | |
| | [WO200125436-A2, 12 APR. 2001] | | | |
| ABU11538 | Human MDDT polypeptide SEQ ID 485 - | 1 . . . 86 | 64/86 (74%) | 1e−26 |
| | Homo sapiens, 100 aa. | 13 . . . 97 | 70/86 (80%) | |
| | [WO200279449-A2, 10 OCT. 2002] | | | |
| AAB81811 | Human endozepine-like ENDO4 SEQ | 3 . . . 86 | 61/84 (72%) | 5e−25 |
| | ID NO: 8 - Homo sapiens, 96 aa. | 11 . . . 93 | 68/84 (80%) | |
| | [WO200125436-A2, 12 APR. 2001] | | | |
| ABJ05397 | Frog acyl coenzyme A binding protein | 4 . . . 86 | 57/83 (68%) | 2e−23 |
| | (ACBP) - Rana sp, 86 aa. | 2 . . . 83 | 66/83 (78%) | |
| | [WO200261096-A1, 08 AUG. 2002] | | | |
| ABJ05396 | Duck acyl coenzyme A binding protein | 4 . . . 86 | 55/83 (66%) | 3e−23 |
| | (ACBP) 2 - Anas sp, 86 aa. | 2 . . . 83 | 66/83 (79%) | |
| | [WO200261096-A1, 08 AUG. 2002] | | | |

In a BLAST search of public sequence databases, the NOV10a protein was found to have homology to the proteins shown in the BLASTP data in Table 10E.

TABLE 10E
Public BLASTP Results for NOV10a

| | | NOV10a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q8N6N7 | Similar to RIKEN cDNA 9230116B18 | 1 . . . 86 | 64/86 (74%) | 4e−26 |
| | gene - Homo sapiens (Human), 88 aa. | 1 . . . 85 | 70/86 (80%) | |
| Q9D258 | 9230116B18Rik protein - Mus musculus | 1 . . . 86 | 60/86 (69%) | 3e−25 |
| | (Mouse), 88 aa. | 1 . . . 85 | 70/86 (80%) | |
| A57711 | diazepam-binding inhibitor - laughing | 1 . . . 86 | 58/86 (67%) | 2e−23 |
| | frog, 88 aa. | 1 . . . 85 | 68/86 (78%) | |
| P45883 | Acyl-CoA-binding protein homolog | 4 . . . 86 | 57/83 (68%) | 4e−23 |
| | (ACBP) (Diazepam binding inhibitor | 3 . . . 84 | 66/83 (78%) | |
| | homolog) (DBI) - Rana ridibunda | | | |
| | (Laughing frog) (Marsh frog), 87 aa. | | | |
| P45882 | Acyl-CoA-binding protein (ACBP) | 4 . . . 86 | 55/83 (66%) | 7e−23 |
| | (Diazepam binding inhibitor) (DBI) | 19 . . . 100 | 66/83 (79%) | |
| | (Endozepine) (EP) - Anas platyrhynchos | | | |
| | (Domestic duck), 103 aa. | | | |

PFam analysis predicts that the NOV10a protein contains the domains shown in the Table 10F.

TABLE 10F
Domain Analysis of NOV10a

| | | Identities/ | |
|---|---|---|---|
| Pfam | | Similarities | Expect |
| Domain | NOV10a Match Region | for the Matched Region | Value |
| ACBP | 3 . . . 86 | 42/90 (47%) | 5.1e−18 |
| | | 66/90 (73%) | |

### Example 11

The NOV11 clone was analyzed, and the nucleotide and encoded polypeptide sequences are shown in Table 11A.

TABLE 11A
NOV11 Sequence Analysis

| | | |
|---|---|---|
| NOV11a, CG55379-04 | SEQ ID NO: 133 | 6291 bp |
| DNA Sequence | ORF Start: ATG at 1 | ORF Stop: TAG at 3763 |

ATGGCGCGGGGGGACGCCGGCCGCGGCCGCGGGCTCCTCGCGTTGACCTTCTGCCTGTTGGCCGCGCGCGGGG
AGCTGCTGTTGCCCCAGGAGACGACTGTGGAGCTGAGCTGTGGAGTGGGGCCACTGCAAGTGATCCTGGGCCC
AGAGCAGGCTGCAGTGCTAAACTGTAGCCTGGGGGCTGCTGCCGCTGGACCCCCCACCAGGGTGACCTGGAGC
AAGGATGGGGACACCCTGCTGGAGCACGACCACTTACACCTGCTGCCCAATGGTTCCCTGTGGCTGTCCGAGC
CACTAGCACCCAATGGCAGTGACGAGTCAGTCCCTGAGGCTGTGGGGGTCATTGAAGGCAACTATTCGTGCCT
AGCCCACGGCCCCCTCGGAGTGCTGGCCAGCCAGACTGCTGTCGTCAAGCTTGCCAGTCTCGCAGACTTCTCT
CTGCACCCGGAGTCTCAGACGGTGGAGGAGAACGGGACAGCTCGCTTTGAGTGCCACATTGAAGGGCTGCCAG
CTCCCATCATTACTTGGGAGAAGGACCAGGTGACATTGCCTGAGGAGCCTCGGCTCATCGTGCTTCCCAACGG
CGTCCTTCAGATCCTGGATGTTCAGGAGAGTGATGCAGGCCCCTACCGCTGCGTGGCCACCAACTCAGCTCGC
CAGCACTTCAGCCAGGAGGCCCTACTCAGTGTGGCCCACAGAGGTTCCCTGGCGTCCACCAGGGGGCAGGACG
TGGTCATTGTGGCAGCCCCAGAGAACACCACAGTGGTGTCTGGCCAGAGTGTGGTGATGGAATGTGTGGCCTC
AGCTGACCCCACCCCTTTTGTGTCCTGGGTCCGAGACGGGAAGCCCATCTCCACAGATGTCATCGTCCTGGGC
CGCACCAACCTACTAATTGCCAACGCGCAGCCCTGGCACTCCGGCGTCTATGTCTGCCGCGCCAACAAGCCCC
GCACGCGCCACTTCGCCACTGCAGCCGCTGAGCTCCGTGTGCTGCTAGCGGCTCCCGCCATCACTCAGGCGCC
CGAGGCGCTGTCGCGGACGCGGGCGAGCACAGCGCGCTTCGTGTGCCGCGCGTCGGGGGAGCCGCGGCCAGCG
CTGCGCTGGCTGCACAACGGGGCGCCGCTGCGGCCCAACGGGCGCGTCAAGGTCCAGGGCGGCGGTGGCAGCC
TGGTCATCACACAGATCGGCCTGCAGGACGCCGGCTACTACCAGTGCGTGGCTGAGAACAGCGCGGGAATGGC
GTGCGCTGCCGCGTCGCTGGCCGTGGTGGTGCGCGAGGGGCTGCCCAGCGCCCCCACGCGGGTCACTGCTACG
CCACTGAGCAGCTCCGCTGTGTTGGTGGCCTGGGAGCGGCCCGAGATGCACAGCGAGCAGATCATCGGCTTCT
CTCTCCACTACCAGAAGGCACGGGGTATGGACAATGTGGAATACCAGTTTGCAGTGAACAACGACACCACAGA
ACTACAGGTTCGGGACCTGGAACCCAACACAGATTATGAGTTCTACGTGGTGGCCTACTCCCAGCTGGGAGCC
AGCCGCACCTCCACCCCAGCACTGGTGCACACACTGGATGATGTCCCCAGTGCAGCACCCCAGCTCTCCCTGT
CCAGCCCCAACCCTTCGGACATCAGGGTGGCGTGGCTGCCCCTGCCCCCCAGCCTGAGCAATGGGCAGGTGGT
GAAGTACAAGATAGAATACGGTTTGGGAAAGGAAGGTGAGTGGGGGGATCAGATTTTCTCTACTGAGGTGCGA
CGAAATGAGACACAGCTTATGCTGAACTCGCTTCAGCCAAACAAGGTGTATCGAGTACGGATTTCGGCTGGTA
CAGCAGCCGGCTTCGGGGCCCCCTCCCAGTGGATGCATCACAGGACGCCCAGTATGCACAACCAGAGCCATGT
CCCTTTTGCCCCTGCAGAGTTGAAGGTGCAGGCAAAGATGGAGTCCCTGGTCGTGTCATGGCAGCCACCCCCT
CACCCCACCCAGATCTCTGGCTACAAACTATATTGGCGGGAGGTGGGGGCTGAGGAGGAGGCCAATGGCGATC
GCCTGCCAGGGGGCCGTGGAGACCAGGCTTGGGATGTGGGGCCTGTCCGGCTCAAGAAGAAAGTGAAGCAGTA
TGAGCTGACCCAGCTAGTCCCTGGCCGGCTGTACGAGGTGAAGCTCGTGGCTTTCAACAAACATGAGGATGGC
TATGCAGCAGTGTGGAAGGGCAAGACGGAGAAGGCGCCGGCACCAGACATGCCTATCCAGAGGGGACCACCCC
TGCCTCCAGCCCACGTCCATGCGGAATCAAACAGCTCCACATCCATCTGGCTTCGGTGGAAAAAGCCAGATTT
CACCACAGTCAAGATTGTCAACTACACTGTGCGCTTCAGCCCCTGGGGGCTCAGGAATGCCTCCCTGGTCACC
TATTACACCAGTTCTGGAGAAGACATCCTCATTGGCGGCTTGAAGCCATTCACCAAATACGAGTTTGCAGTGC
AGTCTCACGGCGTGGACATGGATGGGCCTTTCGGCTCTGTGGTGGAGCGCTCCACCCTGCCTGACCGGCCCTC
CACACCCCCATCCGACCTGCGACTGAGCCCCCTGACACCGTCCACGGTTCGGCTGCACTGGTGCCCCCCCACA
GAGCCCAACGGGGAGATCGTCGAGTATCTGATCCTGTACAGCAGCAACCACACGCAGCCTGAGCACCAGTGGA
CCTTGCTCACCACGCAGGGAAACATCTTCAGTGCTGAGGTCCATGGCCTGGAGAGCGACACTCGGTACTTCTT
CAAGATGGGGGCGCGCACAGAGGTGGGACCTGGGCCTTTCTCCCGCCTGCAGGATGTGATCACGCTCCAGGAG
AAGCTGTCAGACTCGCTGGACATGCACTCAGTCACGGGCATCATCGTGGGTGTCTGCCTGGGCCTCCTCTGCC
TCCTGGCCTGCATGTGTGCTGGCCTGCGCCGCAGCCCCCACAGGGAATCCCTCCCAGGCCTGTCCTCCACCGC
CACCCCCGGGAATCCCGCGCTGTACTCCAGAGCTCGGCTTGGCCCCCCCAGCCCCCCAGCTGCCCATGAATTG
GAGTCCCTTGTGCACCCCCATCCCCAGGACTGGTCCCCGCCACCCTCAGACGTGGAGGACAGGGCTGAAGTGC
ACAGCCTTATGGGTGGCGGTGTTTCTGAAGGCCGGAGTCACTCCAAAAGAAAGATCTCCTGGGCTCAACCAAG
CGGGCTGAGCTGGGCTGGTTCCTGGGCAGGCTGTGAGCTGCCCCAGGCAGGCCCCCGGCCGGCTCTGACCCGG
GCCCTGCTGCCCCCTGCTGGAACTGGGCAGACGCTGTTGCTGCAGGC
TCTGGTGTACGACGCCATAAAGGGCAATGGGAGGAAGAAGTCACCCCCAGCCTGCAGGAACCAGGTGGAGGCT
GAAGTCATTGTCCACTCTGACTTTAGTGCATCTAACGGGAACCCTGACCTCCATCTCCAAGACCTGGAGCCTG
AGGACCCCCTGCCTCCAGAGGCTCCTGATCTCATCTCGGGTGTTGGGGATCCAGGGCAGGGGGCAGCCTGGCT
GGACAGGGAGTTGGGAGGGTGTGAGCTGGCAGCCCCCGGGCCAGACAGACTTACCTGCTTGCCAGAGGCAGCC
AGTGCTTCCTGCTCCTACCCGGACCTCCAGCCAGGCGAGGTGCTAGAGGAGACCCCTGGAGATAGCTGCCAGC
TCAAATCCCCCTGCCCTCTAGGAGCCAGCCCAGGCCTGCCCAGATCCCCGGTCTCCTCCTCTGCCTAGCTCTT
CCCAGAGGATGTGGTTTGGGGCAGGCAGGTATGGATCACATAGGATGCGATACCTGTGGCCGTGTATGTCCAC
ATGTGTGCCTGTAGATACATCATCAAGCCCTTTGGAGCTTCCTAAGTTGCTTTGGCTGAGGGGAGAGGAAAAC
ATGGATTATTCACTCCCCCCATACTCTTTGTGATACACATGTGACATGTGAAAGACATACGAGACATAGCTAC
ATGTGATGTGCACATGTGTGAAGTGCATGTATGCGTACTGGTTGTTGAGCTGGGAAACCGTGGCCCAGGCAGT
GGTCACTACAGCCTGATTGGTCCTCCAGGTCAGAACGGTGCCCCACAGTGGTCAGTCCCCAGCCCTGTGGGCC
CCCACCTCCATCGCCCAGCCTTTTATTACACACTCTGAGAGTGTCTCCAATGCCTGTCTGACAAAGACAGTCC
CAGCCCATTCTCCTGTCTGGCTGGGTTGGGTGCAAGCAGGCTCTGAATGCCTGGCATTTCAGCTGCATCACCT
CCCAGCTCCTTATTGCCCAAATAGAGAGGGTGGCCCTGGCTCCCCTCCGAGCAACTCTGCATTTAATTTTGTA
ATCTGGGAAGTGCCTGGTTTTGAAAATCCGCTTTCTCTCACTCTTCCCCTCCTTCCTTGCCCCTGGCTGCTCT
AGTGTTCTGTCTCCCAGTCACCTCGCTCTCCCAGCACCAGTGCCCTTCTCCTGCTCCCAGATACTCTTTCCTT
TCCTCTCTCCTGTTTTCCTTCCTCTGCTATCTCTCACACCTCTCCCAGACTATGTCATCTTGTTCTCCTGCCT
GGGTTCAAACTCTGCATCCTTCTCTAACAACGTGACTACCTCATGTCTGCTTCAAGGCCCCCGTGCCCTTCCT
GTATCCGCGGCTGCCGCGCACTCGCCTGCCATCCTCCTGCCTCCTCTTCACTCAGTGCTTCTGCTTGCCCTGC
CCCAGGCAGCCCACCCACGCCCAGTCCGGGTGTGGAGAAGATCTTCTGGCTTCCCTGCATCTTGCCTTTGGGA
TTGGGATCCAAGGGTTCTCCATGGATGGATCCAAGTCATAGAGGGGAATGTTTGAGACAGGGAAGGGGGCTGT
GATCCAGAGGCTCAGAATAAAAAGATGCCCTCCCTTCTATGCAGGGGGGCAAGTTTACTGGATGGAGATGATT
TGGGCCTCTCTTCCAGAAGAAGCTAAAGGAAGAGAAGGGGAGTGAGAGTTCAGGGAGGCCCTTCCCACCCTGT
GAGGCTTGACTTGATCTGGATTGGGGATGACAGGAATCTCACCCTCTGGGGTGCTGGCAAGGAGGTCTTTGCA
CAGGAAAAGGGGTAGCTCATTTCAGTTTGTTTTTTCTTTAAATTGAATCCTCAAGTCATTTTCTGTTCACCTG
CCGCACAGGGACAAGCTTGACTTCTATTTTCTGTGTAGTGAAAACAATGTCATTTATTTGGTTTTTCACCTCA
GCCCTCTCATAGGAGCATAGAATGTTAGGGTCTTTACTCCCTAATGATGTCTGATTGGCACATCAAGAGTTAA
CTCTGCCTTCTGGGCCAAATTCGAAATAACCAGTCCATTTTTCCTTTTTTTTTTTTTTTTTTTTTTTTAAATG
GTGGAATGTCTCTCAGCACAGTTGCGGCTTCCTCAAACCCTGAAAGCATCTGTGTTTATTATACTCGGGTGTC
ACTCACTGTTGATGTCTGCACCTACGTTTCCACCTCCTCCCCCTCCTTCAGCCAGCCTATGATAACACTAAAG
ATTATTAATGTTGGTTTTGTATCTCGTTAAAGACAGAATTGTCACTTGTAGTATTTCTGTAGCATTCAGCGCT
GCTGTGGCTAACACCACTGTGTATGTTTCATCATTGCTCTGAAGGTCAAAAGCCTCATTTTATTTTGCTGGTT
TGATTTTTTTTTTTTAAAGAAGAAAAAAAAACTGCCCTGAATTAAATGGCTGTTTTAACAGTAGGCTCTTAGC
ATTATACCACATAGTCATTTTTCATGTTCTTGTTTAACAGGCACTGAGGTTCTGGTTTAAATTAAATAGCTGC
AAATGAGACAATTTATAACCCATTAGGTTGGGTGGAAAATTGTTTCTCAAAAGCAAATAAGTAATAAATCTGG
TATCTGCCTATAACTCACAGTTGATAAGAAAGTGGCCATTTCTCACTAGCACTATATATGATTTGGGCTCTGG
GTAATTTGGAAGTGTTAGGTTTGTGTCTTTGTAGCAGTATTTTTATTAGAAAAGAATCTATTGGCCTTTTACA
GGGTATTAATCCCTTTGTCACCTACCATTGATGCCTTAAGTTTTCTGAGTCTCAATTAAAAATCTTCCTTTTC
TTGATGCATGACAAGTGTAATCAGTACTTGCTCATTTATTTGTCTGTATTTAGTTTATGCTGTACTATTTAAT
TATCCTTCCAGCGTTTTTTTTTTCTCCTTACAAATATGATACTCTTTAGTGTTAAGCTAAGGCATTGATTCAT
GTATCTGTCCTTATAATGAATTAATAAACTATTTTCCAG

| | | | |
|---|---|---|---|
| NOV11a, CG55379-04 | | | |
| Protein Sequence | SEQ ID NO: 134 | 1254 aa | MW at 13408.7kD |

MARGDAGRGRGLLALTFCLLAARGELLLPQETTVELSCGVGPLQVILGPEQAAVLNCSLGAAAAGPPTRVTWS
KDGDTLLEHDHLHLLPNGSLWLSQPLAPNGSDESVPEAVGVIEGNYSCLAHCPLGVLASQTAVVKLASLADFS
LHPESQTVEENGTARFECHIEGLPAPIITWEKDQVTLPEEPRLIVLPNGVLQILDVQESDAGPYRCVATNSAR
QHFSQEALLSVAHRGSLASTRGQDVVIVAAPENTTVVSGQSVVMECVASADPTPFVSWVRDGKPISTDVIVLG
RTNLLIANAQPWHSGVYVCRANKPRTRDFATAAAELRVLLAAPAITQAPEALSRTRASTARFVCRASGEPRPA
LRWLHNGAPLRPNGRVKVQGGGGSLVITQIGLQDAGYYQCVAENSAGMACAAASLAVVVREGLPSAPTRVTAT
PLSSSAVLVAWERPEMHSEQIIGFSLHYQKARGMDNVEYQFAVNNDTTELQVRDLEPNTDYEFYVVAYSQLGA
SRTSTPALVHTLDDVPSAAPQLSLSSPNPSDIRVAWLPLPPSLSNGQVVKYKIEYGLGKEGEWGDQIFSTEVR
GNETQLMLNSLQPNKVYRVRISAGTAAGFGAPSQWMHHRTPSMHNQSHVPFAPAELKVQAKMESLVVSWQPPP
HPTQISGYKLYWREVGAEEEANGDRLPGGRGDQAWDVGPVRLKKKVKQYELTQLVPGRLYEVKLVAFNKHEDG
YAAVWKGKTEKAPAPDMPIQRGPPLPPAHVHAESNSSTSIWLRWKKPDFTTVKIVNYTVRFSPWGLRNASLVT
YYTSSGEDILIGGLKPFTKYEFAVQSHGVDMDGPFGSVVERSTLPDRPSTPPSDLRLSPLTPSTVRLHWCPPT
EPNGEIVEYLILYSSNHTQPEHQWTLLTTQGNIFSAEVHGLESDTRYFFKMGARTEVGPGPFSRLQDVITLQE
KLSDSLDMHSVTGIIVGVCLGLLCLLACMCAGLRRSPHRESLPGLSSTATPGNPALYSRARLGPPSPPAAHEL
ESLVHPHPQDWSPPPSDVEDRAEVHSLMGGGVSEGRSHSKRKISWAQPSGLSWAGSWAGCELPQAGPRPALTR
ALLPPAGTGQTLLLQALVYDAIKGNGRKKSPPACRNOVEAEVIVHSDFSASNGNPDLHLQDLEPEDPLPPEAP
DLISGVGDPGQGAAWLDRELGGCELAAPGPDRLTCLPEAASASCSYPDLQPGEVLEETPGDSCQLKSPCPLGA
SPGLPRSPVSSSA

| | | |
|---|---|---|
| NOV11b, CG55379-01 | SEQ ID NO: 135 | 3741 bp |
| DNA Sequence | ORF Start: ATG at 1 | ORF Stop: end of sequence |

ATGGCGCGGGGGGACGCCGGCCGCGGCCGCGGGCTCCTCGCGTTGACCTTCTGCCTGTTGGCCGCGCGCGGGG
AGCTGCTGTTGCCCCAGGAGACGACTGTGGAGCTGAGCTGTGGAGTGGGGCCACTGCAAGTGATCCTGGGCCC
AGAGCAGGCTGCAGTGCTAAACTGTAGCCTGGGGGCTGCTGCCGCTGGACCCCCCACCAGGGTGACCTGGAGC
AAGGATGGGGACACCCTGCTGGAGCACGACCACTTACACCTGCTGGCCAATGGTTCCCTGTGGCTGTCCCAGC
CACTAGCACCCAATGGCAGTGACGAGTCAGTCCCTGAGGCTGTCGGGGTCATTGAAGGCAACTATTCGTGCCT
ACCCCACGGNCCCCCTCGAGTGCTGGCCAGCCAGACTGCTGTCGTCAAGCTTGCCAGTCTCGCAGACTTCTCT
CTGCACCCGGAGTCTCAGACGGTGGAGGAGAACGGGACAGCTCGCTTTGAGTGCCACATTGAAGGGCTGCCAG
CTCCCATCATTACTTGGGAGAAGGACCAGGTGACATTGCCTGAGGAGCCTCGGCTCATCGTGCTTCCCAACGG
CGTCCTTCAGATCCTGGATGTTCAGGAGAGTGATGCAGGCCCCTACCGCTGCGTGGCCACCAACTCAGCTCGC
CAGCACTTCAGCCAGGAGGCCCTACTCAGTGTGGCCCACAGAGGTTCCCTGGCGTCCACCAGGGGGCAGGACG
TGGTCATTGTGGCAGCCCCAGAGAACACCACAGTGGTGTCTGGCCAGAGTGTGGTGATGGAATGTGTGGCCTC
AGCTGACCCCACCCCTTTTGTGTCCTGGGTCCGAGACGGGAAGCCCATCTCCACAGATGTCATCGTCCTGGGC
CGCACCAACCTACTAATTGCCAACGCGCAGCCCTGGCACTCCGGCGTCTATGTCTGCCGCGCCAACAAGCCCC
GCACGCGCGACTTCGCCACTGCAGCCGCTGAGCTCCGTGTGCTGCTAGCGGCTCCCGCCATCACTCAGGCGCC
CGAGGCGCTGTCGCGGACGCGGGCGAGCACAGCGCGCTTCGTGTGCCGCGCGTCGGGGGAGCCGCGGCCAGCG
CTGCGCTGGCTGCACAACGGGGCGCCGCTGCGGCCCAACGGGCGCGTCAAGGTCCAGGGCGGCGGTGGCAGCC
TGGTCATCACACAGATCGGCCTGCAGGACGCCGGCTACTACCAGTGCGTGGCTGAGAACAGCGCGGGAATGGC
GTGCGCTGCCGCGTCGCTGGCCGTGGTGGTGCGCGAGGGGCTGCCCAGCGCCCCCACGCGGGTCACTGCTACG
CCACTGAGCAGCTCCGCTGTGTTGGTGGCCTGGGAGCGGCCCGAGATGCACAGCGAGCAGATCATCGGCTTCT
CTCTCCACTACCAGAAGGCACGGGGTATGGACAATGTGGAATACCAGTTTGCAGTGAACAACGACACCACAGA
ACTACAGGTTCGGGACCTGGAACCCAACACAGATTATGAGTTCTACGTGGTGGCCTACTCCCAGCTGGGAGCC
AGCCGCACCTCCACCCCAGCACTGGTGCACACACTGGATGATGTCCCCAGTGCAGCACCCCAGCTCTCCCTGT
CCAGCCCCAACCCTTCGGACATCAGGGTGGCGTGGCTGCCCCTGCCCCCCAGCCTGAGCAATGGGCAGGTGGT
GAAGTACAAGATAGAATACGGTTTGGGAAAGGAAGATCAGATTTTCTCTACTGAGGTGCGAGGAAATGAGACA
CAGCTTATGCTGAACTCGCTTCAGCCAAACAAGGTGTATCGAGTACGGATTTCGGCTGGTACAGCAGCCGGCT
TCGGGGCCCCCTCCCAGTGGATGCATCACAGGACGCCCAGTATGCACAACCAGAGCCATGTCCCTTTTGCCCC
TGCAGAGTTGAAGGTGCAGGCAAAGATGGAGTCCCTGGTCGTGTCATGGCAGCCACCCCCTCACCCCACCCAG
ATCTCTGGCTACAAACTATATTGGCGGGAGGTGGGGGCTGAGGAGGAGGCCAATGGCGATCGCCTGCCAGGGG
GCCGTGGAGACCAGGCTTGGGATGTGGGGCCTGTCCGGCTCAAGAAGAAAGTGAAGCAGTATGAGCTGACCCA
GCTAGTCCCTGGCCGGCTGTACGAGGTGAAGCTCGTGGCTTTCAACAAACATGAGGATGGCTATGCAGCAGTG
TGGAAGGGCAAGACGGAGAAGGCGCCGGCACCAGACATGCCTATCCAGAGGGGACCACCCCTGCCTCCAGCCC
ACGTCCATGCGGAATCAAACAGCTCCACATCCATCTGGCTTCGGTGGAAAAAGCCAGATTTCACCACAGTCAA
GATTGTCAACTACACTGTGCGCTTCAGCCCCTGGGGGCTCAGGAATGCCTCCCTGGTCACCTATTACAGTTCT
GGAGAAGACATCCTCATTGGCGGCTTGAAGCCATTCACCAAATACGAGTTTGCAGTGCAGTCTCACGGCGTGG
ACATGGATGGGCCTTTCGGCTCTGTGGTGGAGCGCTCCACCCTGCCTGACCGTCCCTCCACACCCCCATCCGA
CCTGCGACTGAGCCCCCTGACACCGTCCACGGTTCGGCTGCACTGGTGCCCCCCCACAGAGCCCAACGGGGAG
ATCGTGGAGTATCTGATCCTGTACAGCAGCAACCACACGCAGCCTGAGCACCAGTGGACCTTGCTCACCACGC
AGGGTGAGGGAAACATCTTCAGTGCTGAGGTCCATGGCCTGGAGAGCGACACTCGGTACTTCTTCAAGATGGG
GGCGCGCACAGAGGTGGGACCTGGGCCTTTCTCCCGCCTGCAGGATGTGATCACGCTCCAGGAGAAGCTGTCA
GACTCGCTGGACATGCACTCAGTCACGGGCATCATCGTGGGTGTCTGCCTGGGCCTCCTCTGCCTCCTGGCCT
GCATGTGTGCTGGCCTGCGCCGCAGCCCCCACAGGGAATCCCTCCCAGGCCTGTCCTCCACCGCCACCCCCGG
GAATCCCGCGCTGTACTCCAGAGCTCGGCTTGGCCCCCCCAGCCCCCCAGCTGCCCATGAATTGGAGTCCCTT
GTGCACCCCCATCCCCAGGACTGGTCCCCGCCACCCTCAGACGTGGAGGACAGGGCTGAAGTGCACAGCCTTA
TGGGTGGCGGTGTTTCTGAAGGCCGGAGTCACTCCAAAAGAAAGGTAAGTGCTCAACCAAGCGGGCTGAGCTG
GGCTGGTTCCTGGGCAGGCTGTGAGCTGCCCCAGGCAGGCCCCCGGCCGGCTCTGACCCGGGCCCTGCTGCCC
CCTGCTGGAACTGGGCAGACGCTGTTGCTGCAGGTTCTCTGCTCTGA
TCAGGGCAATGGGAGGAAGAAGTCACCCCCAGCCTGCAGGAACCAGGTGGAGGCTGAAGTCATTGTCCACTCT
GACTTTAGTGCATCTAACGGGAACCCTGACCTCCATCTCCAAGACCTGGAGCCTGAGGACCCCCTGCCTCCAG
AGGCTCCTGATCTCATCTCGGGTGTTGGGGATCCAGGGCAGGGGGCAGCCTGGCTGGACAGGGAGTTGGGAGG
GTGTGAGCTGGCAGCCCCCGGGCCAGACAGACTTACCTGCTTGCCAGAGGCAGCCAGTGCTTCCTGCTCCTAC
CCGGACCTCCAGCCAGGCGAGGTGCTAGAGGAGACCCCTGGAGATAGCTGCCAGCTCAAATCCCCCTGCCCTC
TAGGAGCCAGCCCAGGCCTGCCCAGATCCCCGGTCTCCTCCTCT

| | | | |
|---|---|---|---|
| NOV11b, CG55379-01 | | | |
| Protein Sequence | SEQ ID NO: 136 | 1247 aa | MW at 133821.8kD |

MARGDAGRGRGLLALTFCLLAARGELLLPQETTVELSCGVGPLQVILGPEQAAVLNCSLGAAAAGPPTRVTWS
KDGDTLLEHDHLHLLANGSLWLSQPLAPNGSDESVPEAVGVIEGNYSCLAHGPPRVLASQTAVVKLASLADFS
LHPESQTVEENGTARFECHIEGLPAPIITWEKDQVTLPEEPRLIVLPNGVLQILDVQESDAGPYRCVATNSAR
QHFSQEALLSVAHRGSLASTRGQDVVIVAAPENTTVVSGQSVVMECVASADPTPFVSWVRDGKPISTDVIVLG
RTNLLIANAQPWHSGVYVCRANKPRTRDFATAAAELRVLLAAPAITQAPEALSRTRASTARFVCRASGEPRPA
LRWLHNGAPLRPNGRVKVQGGGGSLVITQIGLQDAGYYQCVAENSAGMACAAASLAVVVREGLPSAPTRVTAT
PLSSSAVLVAWERPEMHSEQIIGFSLHYQKARGMDNVEYQFAVNNDTTELQVRDLEPNTDYEFYVVAYSQLGA
SRTSTPALVHTLDDVPSAAPQLSLSSPNPSDIRVAWLPLPPSLSNGQVVKYKIEYGLGKEDQIFSTEVRGNET
QLMLNSLQPNKVYRVRISAGTAAGFGAPSQWMHHRTPSMHNQSHVPFAPAELKVQAKMESLVVSWQPPPHPTQ
ISGYKLYWREVGAEEEANGDRLPGGRGDQAWDVGPVRLKKKVKQYELTQLVPGRLYEVKLVAFNKHEDGYAAV
WKGKTEKAPAPDMPIQRGPPLPPAHVHAESNSSTSIWLRWKKPDFTTVKIVNYTVRFSPWGLRNASLVTYYSS
GEDILIGGLKPFTKYEFAVQSHGVDMDGPFGSVVERSTLPDRPSTPPSDLRLSPLTPSTVRLHWCPPTEPNGE
IVEYLILYSSNHTQPEHQWTLLTTQGEGNIFSAEVHGLESDTRYFFKMGARTEVGPGPFSRLQDVITLQEKLS
DSLDMHSVTGIIVGVCLGLLCLLACMCAGLRRSPHRESLPGLSSTATPGNPALYSRARLGPPSPPAAHELESL
VHPHPQDWSPPPSDVEDRAEVHSLMGGGVSEGRSHSKRKVSAQPSGLSWAGSWAGCELPQAGPRPALTRALLP
PAGTGQTLLLQVLCSDQGNGRKKSPPACRNQVEAEVIVHSDFSASNGNPDLHLQDLEPEDPLPPEAPDLISGV
GDPGQGAAWLDRELGGCELAAPGPDRLTCLPEAASASCSYPDLQPGEVLEETPGDSCQLKSPCPLGASPGLPR
SPVSSS

| | | |
|---|---|---|
| NOV11c, 258065951 | SEQ ID NO: 137 | 1609 bp |
| DNA Sequence | ORF Start: at 1 | ORF Stop: at 1609 |

GGTACCGCGTCGCTGGCCGTGGTGGTGCGCGAGGGGCTGCCCAGCGCCCCCACGCGGGTCACTGCTACGCCAC
TGAGCAGCTCCGCTGTGTTGGTGGCCTGGGAGCGGCCCGAGATGCACAGCGAGCAGATCATCGGCTTCTCTCT
CCACTACCAGAAGGCACGGGGCATGGACAATGTGGAATACCAGTTTGCAGTGAACAACGACACCACAGAACTA
CAGGTTCGGGACCTGGAACCCAACACAGATTATGAGTTCTACGTGGTGGCCTACTCCCAGCTGGGAGCCAGCC
GCACCTCCACCCCAGCACTGGTGCACACACTGGATGATGTCCCCAGTGCAGCACCCCAGCTCTCCCTGTCCAG
CCCCAACCCTTCGGACATCAGGGTGGCGTGGCTGCCCCTGCCCCCCAGCCTGAGCAATGGGCAGGTGGTGAAG
TACAAGATAGAATACGGTTTGGGAAAGGAAGATCAGATTTTCTCTACTGAGGTGCGAGGAAATGAGACACAGC
TTATGCTGAACTCGCTTCAGCCAAACAAGGTGTATCGAGTACGGATTTCGGCTGGTACAGCAGCCGGCTTCGG
GGCCCCCTCCCAGTGGATGCATCACAGGACGCCCAGTATGCACAACCAGAGCCATGTCCCTTTTGCCCCTGCA
GAGTTGAAGGTGCAGGCAAAGATGGAGTCCCTGGTCGTGTCATGGCAGCCACCCCCTCACCCCACCCAGATCT
CTGGCTACAAACTATATTGGCGGGAGGTGGGGGCTGAGGAGGAGGCCAATGGCGATCGCCTGCCAGGGGGCCG
TGGAGACCAGGCTTGGGATGTGGGGCCTCTCCGGCTCAAGAAGAAAGTGAAGCAGTATGAGCTGACCCAGCTA
GTCCCTGGCCGGCTGTACGAGGTGAAGCTCGTGGCTTTCAACAAACATGAGGATGGCTATGCAGCAGTGTGGA
AGGGCAAGACGGAGAAGGCGCCGGCACCAGACATGCCTATCCAGAGGGGACCACCCCTGCCTCCAGCCCACGT
CCATGCGGAATCAAACAGCTCCACATCCATCTGGCTTCGGTGGAAAAAGCCAGATTTCACCACAGTCAAGATT
GTCAACTACACTGTGCGCTTCAGCCCCTGGGGGCTCAGGAATGCCTCCCTGGTCACCTATTACACCAGTTCTG
GAGAAGACATCCTCATTGGCGGCTTGAAGCCATTCACCAAATACGAGTTTGCAGTGCAGTCTCACGGCGTGGA
CATGGATGGGCCTTTCGGCTCTGTGGTGGAGCGCTCCACCCTGCCTGACCGGCCCTCCACACCCCCATCCGAC
CTGCGACTGAGCCCCCTGACACCGTCCACGGTTCGGCTGCACTGCTGCCCCCCCACAGAGCCCAACGGGGAGA
TCGTGGAGTATCTGATCCTGTACAGCAGCAACCACACGCAGCCTGAGCACCAGTGGACCTTGCTCACCACGCA
GGGAAACATCTTCAGTGCTGAGGTCCATGGCCTGGAGAGCGACACTCGGTACTTCTTCAAGATGGGGGCGCGC
ACAGAGGTGGGACCTGGGCCTTTCTCCCGCCTGCAGGATGTGATCACGCTCCAGGAGAAGCTGTCAGACTCGG
TCG

| | | | |
|---|---|---|---|
| NOV11c, 258065951 | | | |
| Protein Sequence | SEQ ID NO: 138 | 536 aa | MW at 59532.7kD |

GTASLAVVVREGLPSAPTRVTATPLSSSAVLVAWERPEMHSEQIIGFSLHYQKARGMDNVEYQFAVNNDTTEL
QVRDLEPNTDYEFYVVAYSQLGASRTSTPALVHTLDDVPSAAPQLSLSSPNPSDIRVAWLPLPPSLSNGQVVK
YKIEYGLGKEDQIFSTEVRGNETQLMLNSLQPNKVYRVRISAGTAAGFGAPSQWMHHRTPSMHNQSHVPFAPA
ELKVQAKMESLVVSWQPPPHPTQISGYKLYWREVGAEEEANGDRLPGGRGDQAWDVGPVRLKKKVKQYELTQL
VPGRLYEVKLVAFNKHEDGYAAVWKGKTEKAPAPDMPIQRGPPLPPAHVHAESNSSTSIWLRWKKPDFTTVKI
VNYTVRFSPWGLRNASLVTYYTSSGEDILIGGLKPFTKYEFAVQSHGVDMDGPFGSVVERSTLPDRPSTPPSD
LRLSPLTPSTVRLHWCPPTEPNGEIVEYLILYSSNHTQPEHQWTLLTTQGNIFSAEVHGLESDTRYFFKMGAR
TEVGPGPFSRLQDVITLQEKLSDSV

| | | |
|---|---|---|
| Nov11d, 209886264 | SEQ ID NO: 139 | 1611 bp |
| DNA Sequence | ORF Start: at 1 | ORF Stop: end of sequence |

GGTACCGCGTCGCTGGCCGTGGTGGTGCGCGAGGGGCTGCCCAGCGCCCCCACGCGGGTCACTGCTACGCCAC
TGAGCAGCTCCGCTGTGTTGGTGGCCTGGGAGCGGCCCGAGATGCCCAGCGAGCAGATCATCGGCTTCTCTCT
CCACTACCAGAAGGCACGGCGCATGGACAATGTGGAATACCAGTTTGCAGTGAACAACGACACCACAGAACTA
CAGGTTCGGGACCTGGAACCCAACACAGATTATGAGTTCTACGTGGTGGCCTACTCCCAGCTGGGAGCCAGCC
GCACCTCCACCCCAGCACTGGTGCACACACTGGATGATGTCCCCAGTGCAGCACCCCAGCTCTCCCTGTCCAG
CCCCAACCCTTCGGACATCAGGGTGGCGTGGCTGCCCCTGCCCCCCAGCCTGAGCAATGGGCAGGTGGTGAAG
TACAAGATAGAATACGGTTTGGGAAAGGAAGATCAGATTTTCTCTACTGAGGTGCGAGGAAATGAGACACAGC
TTATGTTGAACTCGCTTCAGCCAAACAAGGTGTATCGAGTACCGATTTCGGCTGGTACAGCAGCCGGCTTCGG
GGCCCCCTCCCAGTGGATGCATCACAGGACGCCCAGTATGCACAACCAGAGCCATGTCCCTTTTGCCCCTGCA
GAGTTGAAGGTGCAGGCAAAGATGGAGTCCCTGGTCGTATCATGGCAGCCACCCCCTCACCCCACCCAGATCT
CTGGCTACAAACTATATTGGCGGCAGGTGGGGGCTGAGGAGGAGGCCAATGGCGATCGCCTGCCAGGGGGCCG
TGGAGACCAGGCTTGGGATGTGGGGCCTGTCCGGCTCAAGAAGAAAGTGAAGCAGTATGAGCTGACCCAGCTA
GTCCCTGGCCGGCTGTACGAGGTGAAGCTCGTGGCTTTCAACAAACATGAGGATGGCTATGCAGCAGTGTGGA
AGGGCAAGACGGAGAAGGCGCCGGCACCAGACATGCCTATCCAGAGGGGACCACCCCTGCCTCCAGCCCACGT
CCATGCGGAATCAAACAGCTCCACATCCATCTGGCTTCGGTGGAAAAAGCCAGATTTCACCACAGTCAAGATT
GTCAACTACACTGTGCGCTTCAGCCCCTGGGGGCTCAGGAATGCCTCCCTGGTCACCTATTACACCAGTTCTG
GAGAAGACATCCTCATTGGCGGCTTGAAGCCATTCACCAAATACGAGTTTGCAGTGCAGTCTCACGGCGTGGA
CATGGATGGGCCTTTCGGCTCTGTGGTGGAGCGCTCCACCCTGCCTGACCGGCCCTCCACACCCCCATCCGAC
CTGCGACTGAGCCCCCTGACGCCGTCCACGGTTCGGCTGCACTGGTGCCCCCCCACAGAGCCCAACGGGGAGA
TCGTGGAGTATCTGATCCTGTACAGCAGCAACCACACGCAGCCTGAGCACCAGTGGACCTTGCTCACCACGCA
GGGAAACATCTTCAGTGCTGAGGTCCATGGCCTGGAGAGCGACACTCGGTACTTCTTCAAGATGGGGGCGCGC
ACAGAGGTGGGACCTGGGCCTTTCTCCCGCCTGCAGGATGTGATCACGCTCCAGGAGAAGCTGTCAGACTCGG
TCGAC

| | | | |
|---|---|---|---|
| NOV11d, 209886264 | | | |
| Protein Sequence | SEQ ID NO: 140 | 537 aa | MW at 59607.7kD |

GTASLAVVVREGLPSAPTRVTATPLSSSAVLVAWERPEMPSEQIIGFSLHYQKARGMDNVEYQFAVNNDTTEL
QVRDLEPNTDYEFYVVAYSQLGASRTSTPALVHTLDDVPSAAPQLSLSSPNPSDIRVAWLPLPPSLSNGQVVK
YKIEYGLGKEDQIFSTEVRGNETQLMLNSLQPNKVYRVRISAGTAAGFGAPSQWMHHRTPSMHNQSHVPFAPA
ELKVQAKMESLVVSWQPPPHPTQISGYKLYWREVGAEEEANGDRLPGGRGDQAWDVGPVRLKKKVKQYELTQL
VPGRLYEVKLVAFNKHEDGYAAVWKGKTEKAPAPDMPIQRGPPLPPAHVHAESNSSTSIWLRWKKPDFTTVKI
VNYTVRFSPWGLRNASLVTYYTSSGEDILIGGLKPFTKYEFAVQSHGVDMDGPFGSVVERSTLPDRPSTPPSD
LRLSPLTPSTVRLHWCPPTEPNGEIVEYLILYSSNHTQPEHQWTLLTTQGNIFSAEVHGLESDTRYFFKMGAR
TEVGPGPFSRLQDVITLQEKLSDSVD

| | | |
|---|---|---|
| NOV11e, 209886345 | SEQ ID NO: 141 | 1672 bp |
| DNA Sequence | ORF Start: at 1 | ORF Stop: at 1672 |

GGTACCGCGTCGCTGGCCGTGGTGGTGCGCGAGGGGCTGCCCAGCGCCCCCACGCGGGTCACTGCTACGCCAC
TGAGCAGCTCCGCTGTGTTGGTGGCCTGGGAGCGGCCCGAGATGCCCAGCGAGCAGATCATCGGCTTCTCTCT
CCACTACCAGAAGGCACGGGGCATGGACAATGTGGAATACCAGTTTGCAGTGAACAACGACACCACAGAACTA
CAGGTTCGGGACCTGGAACCCAACACAGATTATGAGTTCTACGTGGTGGCCTACTCCCAGCTGGGAGCCAGCC
GCACCTCCACCCCAGCACTGGTGCACACACTGGATGATGTCCCCAGTGCAGCACCCCAGCTCTCCCTGTCCAG
CCCCAACCCTTCGGACATCAGGGTGGCGTGGCTGCCCCTGCCCCCCAGCCTGAGCAATGGGCAGGTGCTGAAG
TACAAGATAGAATACGGTTTGGGAAAGGAAGATCAGATTTTCTCTACTGAGGTGCGAGGAAATGAGACACAGC
TTATGTTGAACTCGCTTCAGCCAAACAAGGTGTATCGAGTACGGATTTCGGCTGGTACAGCAGCCGGCTTCGG
GGCCCCCTCCCAGTGGATGCATCACAGGACGCCCAGTATGCACAACCAGAGCCATGTCCCTTTTGCCCCTGCA
GAGTTGAAGGTGCAGGCAAGGATGGAGTCCCTGGTCGTGTCATGGCAGCCACCCCCTCACCCCACCCAGATCT
CTGGCTACAAACTATATTGGCGGGAGGTGGGGGCTGAGGAGGAGGCCAATGGCGATCGCCTGCCAGGGGGCCG
TGGAGACCAGGCTTGGGATGTGGGGCCTGTCCGGCTCAAGAAGAAAGTGAAGCAGTATGAGCTGACCCAGCTA
GTCCCTGGCCGGCTGTACGAGGTGAAGCTCGTGGCTTTCAACAAACATGAGGATGGCTATGCAGCAGTGTGGA
AGGGCAAGACGGAGAACGCGCCGGCACCAGACATGCCTATCCAGAGGGGACCACCCCTGCCTCCAGCCCACGT
CCATGCGGAATCAAACAGCTCCACATCCATCTGGCTTCGGTGGAAAAAGCCAGATTTCACCACAGTCAAGATT
GTCAACTACACTGTGCGCTTCAGCCCCTGGGGGCTCAGGAATGCCTCCCTGGTCACCTATTACACCAGTTCTG
GAGAAGACATCCTCATTGGCGGCTTGAAGCCATTCACCAAATACGAGTTTGCAGTGCAGTCTCACGGCGTGGA
CATGGATGGGCCTTTCGGCTCTGTGGTGGAGCGCTCCACCCTGCCTGACCGGCCCTCCACACCCCCATCCGAC
CTGCGACTGAGCCCCCTGACGCCGTCCACGGTTCGGCTGCACTGGTGCCCCCCCACAGAGCCCAACGGGGAGA
TCGTGGAGTATCTGATCCTGTACAGCAGCAACCACACGCAGCCTGAGCACCAGTGGACCTTGCTCACCACGCA
GGGAAACATCTTCAGTGCTGAGGTCCATGGCCTGGAGAGCGACACTCGGTACTTCTTCAAGATGGGGGCGCGC
ACAGAGGTGGGACCTGGGCCTTTCTCCCGCCTGCAGGATGTGATCACGCTCCAGGAGAAGCTGTCAGACTCGG
TCGACAGCTTCTCCTGGAGCGTGATCACAGCCCCTCGCGCACCACCACGGCCAGCGACGCGGTACC

| | | | |
|---|---|---|---|
| NOV11e, 209886345 | | | |
| Protein Sequence | SEQ ID NO: 142 | 557 aa | MW at 61878.3kD |

GTASLAVVVREGLPSAPTRVTATPLSSSAVLVAWERPEMPSEQIIGFSLHYQKARGMDNVEYQFAVNNDTTEL
QVRDLEPNTDYEFYVVAYSQLGASRTSTPALVHTLDDVPSAAPQLSLSSPNPSDIRVAWLPLPPSLSNGQVVK
YKIEYGLGKEDQIFSTEVRGNETQLMLNSLQPNKVYRVRISAGTAAGFGAPSQWMHHRTPSMHNQSHVPFAPA
ELKVQARMESLVVSWQPPPHPTQISGYKLYWREVCAEEEANGDRLPGGRGDQAWDVCPVRLKKKVKQYELTQL
VPGRLYEVKLVAFHKHEDGYAAVWKGKTEKAPAPDMPIQRGPPLPPAHVHAESNSSTSIWLRWKKPDFTTVKI
VNYTVRFSPWGLRNASLVTYYTSSGEDILIGGLKPFTKYEFAVQSHGVDMDGPFGSVVERSTLPDRPSTPPSD
LRLSPLTPSTVRLHWCPPTEPNGEIVEYLILYSSNHTQPEHQWTLLTTQGNIFSAEVHGLESDTRYFFKMGAR
TEVGPGPFSRLQDVITLQEKLSDSVDSFSWSVITAPRAPPRPATRY

| | | |
|---|---|---|
| NOV11f, 209886357 | SEQ ID NO: 143 | 1611 bp |
| DNA Sequence | ORF Start: at 1 | ORF Stop: end of sequence |

GGTACCGCGTCGCTGGCCGTGGTGGTGCGCGAGGGGCTGCCCAGCGCCCCCACGCGGGTCACTGCTACGCCAC
TGAGCAGCTCCGCTGTGTTGGTGGCCTGGGAGCGGCCCGAGATGCCCAGCGAGCAGATCATCGGCTTCTCTCT
CCACTACCAGAAGGCACGGGGCATGGACAATGTGGAATACCAGTTTGCAGTGAACAACGACACCACAGAACTA
CAGGTTCGGGACCTGGAACCCAACACAGATTATGAGTTCTACGTGGTGGCCTACTCCCAGCTGGGAGCCAGCC
GCACCTCCACCCCAGCACTGGTGCACACACTGGATGATGTCCCCAGTGCAGCACCCCAGCTCTCCCTGTCCAG
CCCCAACCCTTCGGACATCAGGGTGGCGTGGCTGCCCCTGCCCCCCAGCCTGAGCAATGGGCAGGTGGTGAAG
TACAAGATAGAATACGGTTTGGGAAAGGAAGATCAGATTTTCTCTACTGAGGTGCGAGGAAATGAGACACAGC
TTATGTTGAACTCGCTTCAGCCAAACAAGGTGTATCGAGTACGGATTTCGGCTGGTACAGCAGCCGGCTTCGG
GGCCCCCTCCCAGTGGATGCATCACAGGACGCCCAGTATGCACAACCAGAGCCATGTCCCTTTTGCCCCTGCA
GAGTTGAAGGTGCAGGCAAAGATGGAGTCCCTGGTCGTGTCATGGCAGCCACCCCCTCACCCCACCCAGATCT
CTGGCTACAAACTATATTGGCGGGAGGTGGGGGCTGAGGAGGAGGCCAATGGCGATCGCCTGCCAGGGGGCCG
TGGAGACCAGGCTTGGGATGTGGGGCCTGTCCGGCTCAAGAAGAAAGTGAAGCAGTATGAGCTGACCCAGCTA
GTCCCTGGCCGGCTGTACGAGGTGAAGCTCGTGGCTTTCAACAAACATGAGGATGGCTATGCAGCAGTGTGGA
AGGGCAACACGGAGAAGGCGCCGGCACCAGACATGCCTATCCAGAGGGGACCACCCCTGCCTCCAGCCCACGT
CCATGCGGAATCAAACAGCTCCACATCCATCTGGCTTCGGTGGAAAAAGCCAGATTTCACCACAGTCAAGATT
GTCAACTACACTGTGCGCTTCAGCCCCTGGGGGCTCAGGAATGCCTCCCTGGTCACCTATTACACCAGTTCTG
GAGAAGACATCCTCATTGGCGGCTTGAAGCCATTCACCAAATACGAGTTTGCAGTGCAGTCTCACGGCGTGGA
CATGGATGGGCCTTTCGGCTCTGTGGTGGAGCGCTCCACCCTGCCTGACCGGCCCTCCACACCCCCATCCGAC
CTGCGACTGAGCCCCCTGACGCCGTCCACGGTTCGGCTGCACTGGTGCCCCCCCACAGAGCCCAACGGGGAGA
TCGTGGAGTATCTGATCCTGTACAGCAGCAACCACACGCAGCCTGAGCACCAGTGGACCTTGCTCACCACGCA
GGGAAACATCTTCAGTGCTGAGGTCCATGGCCTGGAGAGCGACACTCGGTACTTCTTCAAGATGGGGGCGCGC
ACAGAGGTGGGACCTGGGCCTTTCTCCCGCCTGCAGGATGTGATCACGCTCCAGGAGAAGCTGTCAGACTCGG
TCGAC

| | | | |
|---|---|---|---|
| NOV11f, 209886357 | | | |
| Protein Sequence | SEQ ID NO: 144 | 537 aa | MW at 59607.7kD |

GTASLAVVVREGLPSAPTRVTATPLSSSAVLVAWERPEMPSEQIIGFSLHYQKARGMDNVEYQFAVNNDTTEL
QVRDLEPNTDYEFYVVAYSQLGASRTSTPALVHTLDDVPSAAPQLSLSSPNPSDIRVAWLPLPPSLSNGQVVK
YKIEYGLGKEDQIFSTEVRGNETQLMLNSLQPNKVYRVRISAGTAAGFGAPSQWMHHRTPSMHNQSHVPFAPA
ELKVQAKMESLVVSWQPPPHPTQISGYKLYWREVGAEEEANGDRLPGGRGDQAWDVGPVRLKKKVKQYELTQL
VPGRLYEVKLVAFNKHEDGYAAVWKGKTEKAPAPDMPIQRGPPLPPAHVHAESNSSTSIWLRWKKPDFTTVKI
VNYTVRFSPWGLRNASLVTYYTSSGEDILIGGLKPFTKYEFAVQSHGVDMDGPFGSVVERSTLPDRPSTPPSD
LRLSPLTPSTVRLHWCPPTEPNGEIVEYLILYSSNHTQPEHQWTLLTTQGNIFSAEVHGLESDTRYFFKMGAR
TEVGPGPFSRLQDVITLQEKLSDSVD

| | | |
|---|---|---|
| NOV11g, CG55379-02 | SEQ ID NO: 145 | 1611 bp |
| DNA Sequence | ORF Start: at 7 | ORF Stop: at 1606 |

GGTACCGCGTCGCTGGCCGTGGTGGTGCGCGAGGGGCTGCCCAGCGCCCCCACGCGGGTCACTGCTACGCCAC
TGAGCAGCTCCGCTGTGTTGGTGGCCTGGGAGCGGCCCGAGATGCCCAGCGAGCAGATCATCGGCTTCTCTCT
CCACTACCAGAAGGCACGCGGCATGGACAATGTGGAATACCAGTTTGCAGTGAACAACGACACCACAGAACTA
CAGGTTCGGGACCTGGAACCCAACACAGATTATGAGTTCTACGTGGTGGCCTACTCCCAGCTGGGAGCCAGCC
GCACCTCCACCCCAGCACTGGTGCACACACTGGATGATGTCCCCAGTGCAGCACCCCAGCTCTCCCTGTCCAG
CCCCAACCCTTCGGACATCAGGGTGGCGTGGCTGCCCCTGCCCCCCAGCCTGAGCAATGGGCAGGTGGTGAAG
TACAAGATAGAATACGGTTTGGGAAAGGAAGATCAGATTTTCTCTACTGAGGTGCGAGGAAATGAGACACAGC
TTATGTTGAACTCGCTTCAGCCAAACAAGGTGTATCGAGTACGGATTTCGGCTGGTACAGCAGCCGGCTTCGG
GGCCCCCTCCCAGTGGATGCATCACAGGACGCCCAGTATGCACAACCAGAGCCATGTCCCTTTTGCCCCTGCA
GAGTTGAAGGTGCAGGCAAAGATGGAGTCCCTGGTCGTATCATGGCAGCCACCCCCTCACCCCACCCAGATCT
CTGGCTACAAACTATATTGGCGGGAGGTGGGGGCTGAGGAGGAGGCCAATGGCGATCGCCTGCCAGGGGGCCG
TGGAGACCAGGCTTGGGATGTGGGGCCTGTCCGGCTCAAGAAGAAAGTGAAGCAGTATGAGCTGACCCAGCTA
GTCCCTGGCCGGCTGTACGAGGTGAAGCTCGTGGCTTTCAACAAACATGAGGATGGCTATGCAGCAGTGTGGA
AGGGCAAGACGGAGAAGGCGCCGGCACCAGACATGCCTATCCAGAGGGGACCACCCCTGCCTCCAGCCCACGT
CCATGCGGAATCAAACAGCTCCACATCCATCTGGCTTCGGTGGAAAAAGCCAGATTTCACCACAGTCAAGATT
GTCAACTACACTGTGCGCTTCAGCCCCTGGGGGCTCAGGAATGCCTCCCTGGTCACCTATTACACCAGTTCTG
GAGAAGACATCCTCATTGGCGGCTTGAAGCCATTCACCAAATACGAGTTTGCAGTGCAGTCTCACGGCGTGGA
CATGGATGGGCCTTTCGGCTCTGTGGTGGAGCGCTCCACCCTGCCTGACCGGCCCTCCACACCCCCATCCGAC
CTGCGACTGAGCCCCCTGACGCCGTCCACGGTTCGGCTGCACTGGTCCCCCCCCACAGAGCCCAACGGGGAGA
TCGTGGAGTATCTGATCCTGTACACCAGCAACCACACGCAGCCTGAGCACCAGTGGACCTTGCTCACCACGCA
GGGAAACATCTTCAGTGCTGAGGTCCATGGCCTGGAGAGCGACACTCGGTACTTCTTCAAGATGGGGGCGCGC
ACAGAGGTGGGACCTGGGCCTTTCTCCCGCCTGCAGGATGTGATCACGCTCCAGGAGAAGCTGTCAGACTCGG
TCGAC

| | | | |
|---|---|---|---|
| NOV11g, CG55379-02 | | | |
| Protein Sequence | SEQ ID NO: 146 | 533 aa | MW at 59235.4kD |

ASLAVVVREGLPSAPTRVTATPLSSSAVLVAWERPEMPSEQIIGFSLHYQKARGMDNVEYQFAVNNDTTELQV
RDLEPNTDYEFYVVAYSQLGASRTSTPALVHTLDDVPSAAPQLSLSSPNPSDIRVAWLPLPPSLSNGQVVKYK
IEYGLGKEDQIFSTEVRGNETQLMLNSLQPNKVYRVRISAGTAAGFGAPSQWMHHRTPSMHNQSHVPFAPAEL
KVQAKMESLVVSWQPPPHPTQISGYKLYWREVGAEEEANGDRLPGGRGDQAWDVGPVRLKKKVKQYELTQLVP
GRLYEVKLVAFNKHEDGYAAVWKGKTEKAPAPDMPIQRGPPLPPAHVHAESNSSTSIWLRWKKPDFTTVKIVN
YTVRFSPWGLRNASLVTYYTSSGEDILIGGLKPFTKYEFAVQSHGVDMDGPFGSVVERSTLPDRPSTPPSDLR
LSPLTPSTVRLHWCPPTEPNGEIVEYLILYSSNHTQPEHQWTLLTTQGNIFSAEVHGLESDTRYFFKMGARTE
VGPGPFSRLQDVITLQEKLSDS

| | | |
|---|---|---|
| NOV11h, CG55379-03 | SEQ ID NO: 147 | 1672 bp |
| DNA Sequence | ORF Start: at 7 | ORF Stop: at 1606 |

GGTACCGCGTCGCTGGCCGTGGTGGTGCGCGAGGGGCTGCCCAGCGCCCCCACGCGGGTCACTGCTACGCCAC
TGAGCAGCTCCGCTGTGTTGGTGGCCTGGGAGCGGCCCGAGATGCCCAGCGAGCAGATCATCGGCTTCTCTCT
CCACTACCAGAGGCACGGGGCATGGACAATGTGGAATACCAGTTTGCAGTGAACAACGACACCACAGAACTA
CAGGTTCGGGACCTGGAACCCAACACAGATTATGAGTTCTACGTGGTGGCCTACTCCCAGCTGGGAGCCAGCC
GCACCTCCACCCCAGCACTGGTGCACACACTGGATGATGTCCCCAGTGCAGCACCCCAGCTCTCCCTGTCCAG
CCCCAACCCTTCGGACATCAGGGTGGCGTGGCTGCCCCTGCCCCCCAGCCTGAGCAATGGGCAGGTGGTGAAG
TACAAGATAGAATACGGTTTGGGAAAGGAAGATCAGATTTTCTCTACTGAGGTGCGAGGAAATGAGACACAGC
TTATGTTGAACTCGCTTCAGCCAAACAAGGTGTATCGAGTACGGATTTCGGCTGGTACAGCAGCCGGCTTCGG
GGCCCCCTCCCAGTGGATGCATCACAGGACGCCCAGTATGCACAACCACAGCCATGTCCCTTTTGCCCCTGCA
GAGTTGAAGGTGCAGGCAAGGATGGAGTCCCTGGTCGTGTCATGGCAGCCACCCCCTCACCCCACCCAGATCT
CTGGCTACAAACTATATTGGCGGGAGGTGGGGGCTGAGGAGGAGGCCAATGGCGATCGCCTGCCAGGGGGCCG
TGGAGACCAGGCTTGGGATGTGGGGCCTGTCCGGCTCAAGAAGAAAGTGAAGCAGTATGAGCTGACCCAGCTA
GTCCCTGGCCGGCTGTACGAGGTGAAGCTCGTGGCTTTCAACAAACATGAGGATGGCTATGCAGCAGTGTGGA
AGGGCAAGACGCAGAAGGCGCCGGCACCAGACATGCCTATCCAGAGGGGACCACCCCTGCCTCCAGCCCACGT
CCATGCGGAATCAAACAGCTCCACATCCATCTGGCTTCCGTGGAAAAAGCCAGATTTCACCACAGTCAAGATT
GTCAACTACACTGTGCGCTTCAGCCCCTGGGGGCTCAGGAATGCCTCCCTGGTCACCTATTACACCAGTTCTG
GAGAAGACATCCTCATTGGCGGCTTGAAGCCATTCACCAAATACGAGTTTGCAGTGCAGTCTCACGGCGTGGA
CATGGATGGGCCTTTCGGCTCTGTGGTGGAGCGCTCCACCCTGCCTGACCGGCCCTCCACACCCCCATCCGAC
CTGCGACTGACCCCCCTGACGCCGTCCACGGTTCGGCTGCACTGGTGCCCCCCCACAGAGCCCAACGGGGAGA
TCGTGGAGTATCTGATCCTGTACAGCAGCAACCACACGCAGCCTGAGCACCAGTGGACCTTGCTCACCACGCA
GGGAAACATCTTCAGTGCTGAGGTCCATGGCCTGGAGAGCGACACTCGGTACTTCTTCAAGATGGGGGCGCGC
ACAGAGGTGGGACCTGGGCCTTTCTCCCGCCTGCAGGATGTGATCACGCTCCAGGAGAAGCTGTCAGACTCGG
TCGACAGCTTCTCCTGGAGCGTGATCACAGCCCCTCGCGCACCACCACGGCCAGCGACGCGGTACC

| | | | |
|---|---|---|---|
| NOV11h, CG55379-03 | | | |
| Protein Sequence | SEQ ID NO: 148 | 533 aa | MW at 59263.4kD |

ASLAVVVREGLPSAPTRVTATPLSSSAVLVAWERPEMPSEQIIGFSLHYQKARGMDNVEYQFAVNNDTTELQV
RDLEPNTDYEFYVVAYSQLGASRTSTPALVHTLDDVPSAAPQLSLSSPNPSDIRVAWLPLPPSLSNGQVVKYK
IEYGLGKEDQIFSTEVRGNETQLMLNSLQPNKVYRVRISAGTAAGFGAPSQWMHHRTPSMHNQSHVPFAPAEL
KVQARMESLVVSWQPPPHPTQISGYKLYWREVGAEEEANGDRLPGGRGDQAWDVGPVRLKKKVKQYELTQLVP
GRLYEVKLVAFNKHEDGYAAVWKGKTEKAPAPDMPIQRGPPLPPAHVHAESNSSTSIWLRWKKPDFTTVKIVN
YTVRFSPWGLRNASLVTYYTSSGEDILIGGLKPFTKYEFAVQSHGVDMDGPFGSVVERSTLPDRPSTPPSDLR
LSPLTPSTVRLHWCPPTEPNGEIVEYLILYSSNHTQPEHQWTLLTTQGNIFSAEVHGLESDTRYFFKMGARTE
VGPGPFSRLQDVITLQEKLSDS

A ClustalW comparison of the above protein sequences yields the following sequence alignment shown in Table 11B.

TABLE 11B
Comparison of the NOV11 protein sequences.

| | |
|---|---|
| NOV11a | MARGDAGRGRGLLALTFCLLAARGELLLPQETTVELSCGVGPLQVILGPEQAAVLNCSLG |
| NOV11b | MARGDAGRGRGLLALTFCLLAARGELLLPQETTVELSCGVGPLQVILGPEQAAVLNCSLG |
| NOV11c | ------------------------------------------------------------ |
| NOV11d | ------------------------------------------------------------ |
| NOV11e | ------------------------------------------------------------ |
| NOV11f | ------------------------------------------------------------ |
| NOV11g | ------------------------------------------------------------ |
| NOV11h | ------------------------------------------------------------ |
| NOV11a | AAAAGPPTRVTWSKDGDTLLEHDHLHLLPNGSLWLSQPLAPNGSDESVPEAVGVIEGNYS |
| NOV11b | AAAAGPPTRVTWSKDGDTLLEHDHLHLLANGSLWLSQPLAPNGSDESVPEAVGVIEGNYS |
| NOV11c | ------------------------------------------------------------ |
| NOV11d | ------------------------------------------------------------ |
| NOV11e | ------------------------------------------------------------ |
| NOV11f | ------------------------------------------------------------ |
| NOV11g | ------------------------------------------------------------ |
| NOV11h | ------------------------------------------------------------ |
| NOV11a | CLAHGPLGVLASQTAVVKLASLADFSLHPESQTVEENGTARFECHIEGLPAPIITWEKDQ |
| NOV11b | CLAHGPPRVLASQTAVVKLASLADFSLHPESQTVEENGTARFECHIEGLPAPIITWEKDQ |
| NOV11c | ------------------------------------------------------------ |
| NOV11d | ------------------------------------------------------------ |
| NOV11e | ------------------------------------------------------------ |
| NOV11f | ------------------------------------------------------------ |
| NOV11g | ------------------------------------------------------------ |
| NOV11h | ------------------------------------------------------------ |
| NOV11a | VTLPEEPRLIVLPNGVLQILDVQESDAGPYRCVATNSARQHFSQEALLSVAHRGSLASTR |
| NOV11b | VTLPEEPRLIVLPNGVLQILDVQESDAGPYRCVATNSARQHFSQEALLSVAHRGSLASTR |
| NOV11c | ------------------------------------------------------------ |
| NOV11d | ------------------------------------------------------------ |
| NOV11e | ------------------------------------------------------------ |
| NOV11f | ------------------------------------------------------------ |
| NOV11g | ------------------------------------------------------------ |
| NOV11h | ------------------------------------------------------------ |
| NOV11a | GQDVVIVAAPENTTVVSGQSVVMECVASADPTPFVSWVRDGKPISTDVIVLGRTNLLIAN |
| NOV11b | GQDVVIVAAPENTTVVSGQSVVMECVASADPTPFVSWVRDGKPISTDVIVLGRTNLLIAN |
| NOV11c | ------------------------------------------------------------ |
| NOV11d | ------------------------------------------------------------ |
| NOV11e | ------------------------------------------------------------ |
| NOV11f | ------------------------------------------------------------ |
| NOV11g | ------------------------------------------------------------ |
| NOV11h | ------------------------------------------------------------ |
| NOV11a | AQPWHSGVYVCRANKPRTRDFATAAAELRVLLAAPAITQAPEALSRTRASTARFVCRASC |
| NOV11b | AQPWHSGVYVCRANKPRTRDFATAAAELRVLLAAPAITQAPEALSRTRASTARFVCRASG |
| NOV11c | ------------------------------------------------------------ |
| NOV11d | ------------------------------------------------------------ |
| NOV11e | ------------------------------------------------------------ |
| NOV11f | ------------------------------------------------------------ |
| NOV11g | ------------------------------------------------------------ |
| NOV11h | ------------------------------------------------------------ |
| NOV11a | EPRPALRWLHNGAPLRPNGRVKVQGGGGSLVITQIGLQDAGYYQCVAENSAGMACAAASL |
| NOV11b | EPRPALRWLHNGAPLRPNGRVKVQGGGGSLVITQIGLQDAGYYQCVAENSAGMACAAASL |
| NOV11c | -------------------------------------------------------GTASL |
| NOV11d | -------------------------------------------------------GTASL |
| NOV11e | -------------------------------------------------------GTASL |
| NOV11f | -------------------------------------------------------GTASL |
| NOV11g | ---------------------------------------------------------ASL |
| NOV11h | ---------------------------------------------------------ASL |
| NOV11a | AVVVREGLPSAPTRVTATPLSSSAVLVAWERPEMHSEQIIGFSLHYQKARGMDNVEYQFA |
| NOV11b | AVVVREGLPSAPTRVTATPLSSSAVLVAWERPEMHSEQIIGFSLHYQKARGMDNVEYQFA |
| NOV11c | AVVVREGLPSAPTRVTATPLSSSAVLVAWERPEMHSEQIIGFSLHYQKARGMDNVEYQFA |
| NOV11d | AVVVREGLPSAPTRVTATPLSSSAVLVAWERPEMPSEQIIGFSLHYQKARGMDNVEYQFA |
| NOV11e | AVVVREGLPSAPTRVTATPLSSSAVLVAWERPEMPSEQIIGFSLHYQKARGMDNVEYQFA |
| NOV11f | AVVVREGLPSAPTRVTATPLSSSAVLVAWERPEMPSEQIIGFSLHYQKARGMDNVEYQFA |
| NOV11g | AVVVREGLPSAPTRVTATPLSSSAVLVAWERPEMPSEQIIGFSLHYQKARGMDNVEYQFA |
| NOV11h | AVVVREGLPSAPTRVTATPLSSSAVLVAWERPEMPSEQIIGFSLHYQKARGMDNVEYQFA |
| NOV11a | VNNDTTELQVRDLEPNTDYEFYVVAYSQLGASRTSTPALVHTLDDVPSAAPQLSLSSPNP |
| NOV11b | VNNDTTELQVRDLEPNTDYEFYVVAYSQLGASRTSTPALVHTLDDVPSAAPQLSLSSPNP |
| NOV11c | VNNDTTELQVRDLEPNTDYEFYVVAYSQLGASRTSTPALVHTLDDVPSAAPQLSLSSPNP |
| NOV11d | VNNDTTELQVRDLEPNTDYEFYVVAYSQLGASRTSTPALVHTLDDVPSAAPQLSLSSPNP |
| NOV11e | VNNDTTELQVRDLEPNTDYEFYVVAYSQLGASRTSTPALVHTLDDVPSAAPQLSLSSPNP |
| NOV11f | VNNDTTELQVRDLEPNTDYEFYVVAYSQLGASRTSTPALVHTLDDVPSAAPQLSLSSPNP |
| NOV11g | VNNDTTELQVRDLEPNTDYEFYVVAYSQLGASRTSTPALVHTLDDVPSAAPQLSLSSPNP |
| NOV11h | VNNDTTELQVRDLEPNTDYEFYVVAYSQLGASRTSTPALVHTLDDVPSAAPQLSLSSPNP |
| NOV11a | SDIRVAWLPLPPSLSNGQVVKYKIEYGLGKEGEWGDQIFSTEVRGNETQLMLNSLQPNKV |
| NOV11b | SDIRVAWLPLPPSLSNGQVVKYKIEYGLGKE----DQIFSTEVRGNETQLMLNSLQPNKV |
| NOV11c | SDIRVAWLPLPPSLSNGQVVKYKIEYGLGKE----DQIFSTEVRGNETQLMLNSLQPNKV |
| NOV11d | SDIRVAWLPLPPSLSNGQVVKYKIEYGLGKE----DQIFSTEVRGNETQLMLNSLQPNKV |
| NOV11e | SDIRVAWLPLPPSLSNGQVVKYKIEYGLGKE----DQIFSTEVRGNETQLMLNSLQPNKV |
| NOV11f | SDIRVAWLPLPPSLSNGQVVKYKIEYGLGKE----DQIFSTEVRGNETQLMLNSLQPNKV |
| NOV11g | SDIRVAWLPLPPSLSNGQVVKYKIEYGLGKE----DQIFSTEVRGNETQLMLNSLQPNKV |
| NOV11h | SDIRVAWLPLPPSLSNGQVVKYKIEYGLGKE----DQIFSTEVRGNETQLMLNSLQPNKV |
| NOV11a | YRVRISAGTAAGFGAPSQWMHHRTPSMHNQSHVPFAPAELKVQAKMESLVVSWQPPPHPT |
| NOV11b | YRVRISAGTAAGFGAPSQWMHHRTPSNHNQSHVPFAPAELKVQAKMESLVVSWQPPPHPT |
| NOV11c | YRVRISAGTAAGFGAPSQWMHHRTPSMHNQSHVPFAPAELKVQAKMESLVVSWQPPPHPT |
| NOV11d | YRVRISAGTAAGFGAPSQWMHHRTPSMHNQSHVPFAPAELKVQAKMESLVVSWQFPPHPT |
| NOV11e | YRVRISAGTAAGFGAPSQWMHHRTPSMHNQSHVPFAPAELKVQARMESLVVSWQPPPHPT |
| NOV11f | YRVRISAGTAAGFGAPSQWMHHRTPSMHNQSNVPFAPAELKVQAKMESLVVSWQPPPHPT |
| NOV11g | YRVRISAGTAAGFGAPSQWMHHRTPSMHNQSHVPFAPAELKVQAKMESLVVSWQPPPHPT |
| NOV11h | YRVRISAGTAAGFGAPSQWMHHRTPSMHNQSHVPFAPAELKVQARMESLVVSWQPPPHPT |
| NOV11a | QISGYKLYWREVGAEEEANGDRLPGGRGDQAWDVGPVRLKKKVKQYELTQLVPGRLYEVK |
| NOV11b | QISGYKLYWREVGAEEEANGDRLPGGRGDQAWDVGPVRLKKKVKQYELTQLVPGRLYEVK |
| NOV11c | QISGYKLYWREVGAEEEANGDRLPGGRGDQAWDVGPVRLKKKVKQYELTQLVPGRLYEVK |
| NOV11d | QISGYKLYWREVGAEEEANGDRLPGGRGDQAWDVGPVRLKKKVKQYELTQLVPGRLYEVK |
| NOV11e | QISGYKLYWREVGAEEEANGDRLPGGRGDQAWDVGPVRLKKKVKQYELTQLVPGRLYEVK |
| NOV11f | QISGYKLYWREVGAEEEANGDRLPGGRGDQAWDVGPVRLKKKVKQYELTQLVPGRLYEVK |
| NOV11g | QISGYKLYWREVGAEEEANGDRLPGGRGDQAWDVGPVRLKKKVKQYELTQLVPGRLYEVK |
| NOV11h | QISCYKLYWREVGAEEEANGDRLPGGRGDQAWDVGPVRLKKKVKQYELTQLVPGRLYEVK |
| NOV11a | LVAFNKHEDGYAAVWKGKTEKAPAPDMPIQRGPPLPPAHVHAESNSSTSIWLRWKKPDFT |
| NOV11b | LVAFNKHEDGYAAVWKGKTEKAPAPDMPIQRGPPLPPAHVHAESNSSTSIWLRWKKPDFT |
| NOV11c | LVAFNKHEDGYAAVWKGKTEKAPAPDMPIQRGPPLPPAHVHAESNSSTSIWLRWKKPDFT |
| NOV11d | LVAFNKHEDGYAAVWKGKTEKAPAPDMPIQRGPPLPPAHVHAESNSSTSIWLRWKKPDFT |
| NOV11e | LVAFNKHEDGYAAVWKGKTEKAPAPDMPIQRGPPLPPAHVHAESNSSTSIWLRWKKPDFT |
| NOV11f | LVAFNKHEDGYAAVWKGKTEKAPAPDMPIQRGPPLPPAHVHAESNSSTSIWLRWKKPDFT |
| NOV11g | LVAFNKHEDGYAAVWKGKTEKAPAPDMPIQRGPPLPPAHVHAESNSSTSIWLRWKKPDFT |
| NOV11h | LVAFNKHEDGYAAVWKGKTEKAPAPDMPIQRGPPLPPAHVHAESNSSTSIWLRWKKPDFT |
| NOV11a | TVKIVNYTVRFSPWGLRNASLVTYYTSSGEDILIGGLKPFTKYEFAVQSHGVDMDGPFGS |
| NOV11b | TVKIVNYTVRFSPWGLRNASLVTYYSS-GEDILIGGLKPFTKYEFAVQSHGVDMDGPFGS |
| NOV11c | TVKIVNYTVRFSPWGLRNASLVTYYTSSGEDILIGGLKPFTKYEFAVQSHGVDMDGPFGS |
| NOV11d | TVKIVNYTVRFSPWGLRNASLVTYYTSSGEDILIGGLKPFTKYEFAVQSHGVDMDGPFGS |
| NOV11e | TVKIVNYTVRFSPWGLRNASLVTYYTSSGEDILIGGLKPFTKYEFAVQSHGVDMDGPFGS |
| NOV11f | TVKIVNYTVRFSPWGLRNASLVTYYTSSGEDILIGGLKPFTKYEFAVQSHGVDMDGPFGS |
| NOV11g | TVKIVNYTVRFSPWGLRNASLVTYYTSSGEDILIGGLKPFTKYEFAVQSHGVDMDGPFGS |
| NOV11h | TVKIVNYTVRFSPWGLRNASLVTYYTSSGEDILIGGLKPFTKYEFAVQSHGVDMDGPFGS |
| NOV11a | VVERSTLPDRPSTPPSDLRLSPLTPSTVRLHWCPPTEPNGEIVEYLILYSSNHTQPEHQW |
| NOV11b | VVERSTLPDRPSTPPSDLRLSPLTPSTVRLHWCPPTEPNGEIVEYLILYSSNHTQPENQW |
| NOV11c | VVERSTLPDRPSTPPSDLRLSPLTPSTVRLHWCPPTEPNGEIVEYLILYSSNHTQPEHQW |
| NOV11d | VVERSTLPDRPSTPPSDLRLSPLTPSTVRLHWCPPTEPNGEIVEYLILYSSNHTQPEHQW |
| NOV11e | VVERSTLPDRPSTPPSDLRLSPLTPSTVRLHWCPPTEPNGEIVEYLILYSSNHTQPEHQW |
| NOV11f | VVERSTLPDRPSTPPSDLRLSPLTPSTVRLHWCPPTEPNGEIVEYLILYSSNHTQPEHQW |
| NOV11g | VVERSTLPDRPSTPPSDLRLSPLTPSTVRLHWCPPTEPNGEIVEYLILYSSNHTQPEHQW |
| NOV11h | VVERSTLPDRPSTPPSDLRLSPLTPSTVRLHWCPPTEPNGEIVEYLILYSSNNTQPEHQW |
| NOV11a | TLLTTQG--NIFSAEVHGLESDTRYFFKMGARTEVGPGPFSRLQDVITLQEKLSDSLDMH |
| NOV11b | TLLTTQGEGNIFSAEVHGLESDTRYFFKMGARTEVGPGPFSRLQDVITLQEKLSDSLDMH |
| NOV11c | TLLTTQG--NIFSAEVHGLESDTRYFFKMGARTEVGPGPFSRLQDVITLQEKLSDSV--- |
| NOV11d | TLLTTQG--NIFSAEVHGLESDTRYFFKMGARTEVGPGPFSRLQDVITLQEKLSDSVD-- |
| NOV11e | TLLTTQG--NIFSAEVHGLESDTRYFFKMGARTEVGPGPFSRLQDVITLQEKLSDSVDSF |
| NOV11f | TLLTTQC--NIFSAEVHGLESDTRYFFKMGARTEVGPGPFSRLQDVITLQEKLSDSVD-- |
| NOV11g | TLLTTQG--NIFSAEVHGLESDTRYFFKMGARTEVGPGPFSRLQDVITLQEKLSDS---- |
| NOV11h | TLLTTQG--NIFSAEVHGLESDTRYFFKMGARTEVGPGPFSRLQDVITLQEKLSDS---- |
| NOV11a | SVTGIIVGVCLGLLCLLACMCAGLRRSPHRESLPGLSSTATPGNPALYSRARLGPPSPPA |
| NOV11b | SVTGIIVGVCLGLLCLLACMCAGLRRSPHRESLPGLSSTATPGNPALYSRARLGPPSPPA |
| NOV11c | ------------------------------------------------------------ |
| NOV11d | ------------------------------------------------------------ |
| NOV11e | SWSVITAPRAPPRPATRY------------------------------------------ |
| NOV11f | ------------------------------------------------------------ |
| NOV11g | ------------------------------------------------------------ |
| NOV11h | ------------------------------------------------------------ |
| NOV11a | AHELESLVHPHPQDWSPPPSDVEDRAEVHSLMGGGVSEGRSHSKRKISWAQPSGLSWAGS |
| NOV11b | AHELESLVHPHPQDWSPPPSDVEDRAEVHSLMGGGVSEGRSHSKRK-VSAQPSGLSWAGS |
| NOV11c | ------------------------------------------------------------ |
| NOV11d | ------------------------------------------------------------ |
| NOV11e | ------------------------------------------------------------ |
| NOV11f | ------------------------------------------------------------ |
| NOV11g | ------------------------------------------------------------ |
| NOV11h | ------------------------------------------------------------ |
| NOV11a | WAGCELPQAGPRPALTRALLPPAGTGQTLLLQALVYDAIKGNGRKKSPPACRNQVEAEVI |
| NOV11b | WAGCELPQAGPRPALTRALLPPAGTGQTLLLQVLCSD--QGNGRKKSPPACRNQVEAEVI |
| NOV11c | ------------------------------------------------------------ |
| NOV11d | ------------------------------------------------------------ |
| NOV11e | ------------------------------------------------------------ |
| NOV11f | ------------------------------------------------------------ |
| NOV11g | ------------------------------------------------------------ |
| NOV11h | ------------------------------------------------------------ |
| NOV11a | VHSDFSASNGNPDLHLQDLEPEDPLPPEAPDLISGVGDPGQGAAWLDRELGGCELAAPGP |
| NOV11b | VHSDFSASNGNPDLHLQDLEPEDPLPPEAPDLISGVGDPGQGAAWLDRELGGCELAAPGP |
| NOV11c | ------------------------------------------------------------ |
| NOV11d | ------------------------------------------------------------ |
| NOV11e | ------------------------------------------------------------ |
| NOV11f | ------------------------------------------------------------ |
| NOV11g | ------------------------------------------------------------ |
| NOV11h | ------------------------------------------------------------ |
| NOV11a | DRLTCLPEAASASCSYPDLQPGEVLEETPGDSCQLKSPCPLGASPGLPRSPVSSSA |
| NOV11b | DRLTCLPEAASASCSYPDLQPGEVLEETPGDSCQLKSPCPLGASPGLPRSPVSSS- |
| NOV11c | ------------------------------------------------------------ |
| NOV11d | ------------------------------------------------------------ |
| NOV11e | ------------------------------------------------------------ |
| NOV11f | ------------------------------------------------------------ |
| NOV11g | ------------------------------------------------------------ |
| NOV11h | ------------------------------------------------------------ |
| NOV11a | (SEQ ID NO: 134) |
| NOV11b | (SEQ ID NO: 136) |
| NOV11c | (SEQ ID NO: 138) |
| NOV11d | (SEQ ID NO: 140) |
| NOV11e | (SEQ ID NO: 142) |
| NOV11f | (SEQ ID NO: 144) |
| NOV11g | (SEQ ID NO: 146) |
| NOV11h | (SEQ ID NO: 148) |

Further analysis of the NOV11a protein yielded the following properties shown in Table 11C.

TABLE 11C
Protein Sequence Properties NOV11a

| SignalP | Cleavage site between residues 25 and 26 | | | |
|---|---|---|---|---|
| analysis: | | | | |
| PSORT II | PSG: a new signal peptide prediction method | | | |
| analysis: | N-region: length 10; pos. chg 3; neg. chg 1 | | | |
| | H-region: length 12; peak value 10.30 | | | |
| | PSG score: 5.90 | | | |
| | GvH: von Heijne's method for signal seq. recognition | | | |
| | GvH score (threshold: −2.1): 1.11 | | | |
| | possible cleavage site: between 24 and 25 | | | |
| | >>> Seems to have a cleavable signal peptide (1 to 24) | | | |
| | ALOM: Klein et al's method for TM region allocation | | | |
| | Init position for calculation: 25 | | | |
| | Tentative number of TMS(s) for the threshold 0.5: 1 | | | |
| | Number of TMS(s) for threshold 0.5: 1 | | | |
| | INTEGRAL | Likelihood = | −11.89 | Transmembrane 963-979 |
| | PERIPHERAL | Likelihood = | 0.90 (at 321) | |
| | ALOM score: −11.89 (number of TMSs: 1) | | | |
| | MTOP: Prediction of membrane topology (Hartmann et al.) | | | |
| | Center position for calculation: 12 | | | |
| | Charge difference: −5.0 C(−2.0)-N(3.0) | | | |
| | N >= C: N-terminal side will be inside | | | |
| | >>> membrane topology: type 1a (cytoplasmic tail 980 to 1254) | | | |
| | MITDISC: discrimination of mitochondrial targeting seq | | | |
| | R content: | 4 | Hyd Moment(75): | 11.83 |
| | Hyd Moment(95): | 5.63 | G content: | 5 |
| | D/E content: | 2 | S/T content: | 1 |

Score: −4.74
Gavel: prediction of cleavage sites for mitochondrial preseq
R-2 motif at 33 ARG|EL
NUCDISC: discrimination of nuclear localization signals
pat4: none
pat7: PVRLKKK (4) at 696
bipartite: none
content of basic residues: 8.2%
NLS Score: −0.13
KDEL: ER retention motif in the C-terminus: none
ER Membrane Retention Signals:
XXRR-like motif in the N-terminus: ARGD
none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: too long tail
Dileucine motif in the tail: found
LL at 1097
LL at 1107
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 55.5
COIL: Lupas's algorithm to detect coiled-coil regions
total: 0 residues
Final Results (k = 9/23):
55.6%: endoplasmic reticulum
22.2%: Golgi
11.1%: plasma membrane
11.1%: extracellular, including cell wall
>> prediction for CG55379-04 is end (k = 9)

A search of the NOV11a protein against the Geneseq database, a proprietary database that contains sequences published in patents and patent publication, yielded several homologous proteins shown in Table 11D.

TABLE 11D
Geneseq Results for NOV11a

| | | NOV11a | | |
|---|---|---|---|---|
| | | Residues/ | Identities/ | |
| Geneseq | Protein/Organism/Length [Patent | Match | Similarities for the | Expect |
| Identifier | #,Date] | Residues | Matched Region | Value |
| AAG65914 | Amino acid sequence of GSK gene Id | 1 . . . 1254 | 1248/1255 (99%) | 0.0 |
| | 27142 - Homo sapiens, 1250 aa. | 1 . . . 1250 | 1249/1255 (99%) | |
| | [WO200172961-A2, 04 OCT. 2001] | | | |
| AAU77405 | Human NOV1 protein, homologue of | 1 . . . 1253 | 1237/1255 (98%) | 0.0 |
| | NOPE/PUNC Ig proteins - Homo | 1 . . . 1247 | 1239/1255 (98%) | |
| | sapiens, 1247 aa. | | | |
| | [WO200206329-A2, 24 JAN. 2002] | | | |
| AAE05251 | Mouse Nope (neighbour of punc ell) | 1 . . . 1253 | 1093/1256 (87%) | 0.0 |
| | protein - Mus musculus, 1252 aa. | 1 . . . 1249 | 1146/1256 (91%) | |
| | [WO200149714-A2, 12 JUL. 2001] | | | |
| ABP69002 | Human polypeptide SEQ ID NO | 332 . . . 1254 | 919/923 (99%) | 0.0 |
| | 1049 - Homo sapiens, 980 aa. | 62 . . . 980 | 919/923 (99%) | |
| | [WO200270539-A2, 12 SEP. 2002] | | | |
| AAE05252 | Mouse Nope (neighbour of punc ell) | 24 . . . 958 | 843/937 (89%) | 0.0 |
| | extracellular domain - Mus musculus, | 1 . . . 932 | 879/937 (92%) | |
| | 932 aa. [WO200149714-A2, | | | |
| | 12 JUL. 2001] | | | |

In a BLAST search of public sequence databases, the NOV11a protein was found to have homology to the proteins shown in the BLASTP data in Table 11E.

TABLE 11E
Public BLASTP Results for NOV11a

| | | NOV11a | | |
|---|---|---|---|---|
| Protein | | Residues/ | Identities/ | |
| Accession | | Match | Similarities for the | Expect |
| Number | Protein/Organism/Length | Residues | Matched Portion | Value |
| Q8TDY8 | HDDM36 - Homo sapiens | 1 . . . 1254 | 1248/1255 (99%) | 0.0 |
| | (Human), 1250 aa. | 1 . . . 1250 | 1249/1255 (99%) | |
| Q9EQS9 | DDM36 - Mus musculus (Mouse), | 1 . . . 1253 | 1100/1256 (87%) | 0.0 |
| | 1252 aa. | 1 . . . 1249 | 1151/1256 (91%) | |
| Q9EQS8 | DDM36E - Mus musculus (Mouse), | 1 . . . 1253 | 1100/1256 (87%) | 0.0 |
| | 1253 aa. | 1 . . . 1250 | 1151/1256 (91%) | |
| Q9JLI1 | Neighbor of Punc ell protein - Mus | 1 . . . 1253 | 1093/1256 (87%) | 0.0 |
| | musculus (Mouse), 1252 aa. | 1 . . . 1249 | 1146/1256 (91%) | |
| Q9HCE4 | Hypothetical protein KIAA1628 - | 332 . . . 1254 | 919/923 (99%) | 0.0 |
| | Homo sapiens (Human), 980 aa | 62 . . . 980 | 919/923 (99%) | |
| | (fragment). | | | |

PFam analysis predicts that the NOV11a protein contains the domains shown in the Table 11F.

TABLE 11F
Domain Analysis of NOV11a

| | | Identities/ | |
|---|---|---|---|
| | | Similarities | |
| Pfam | | for the | Expect |
| Domain | NOV11a Match Region | Matched Region | Value |
| ig | 157 . . . 214 | 15/61 (25%) | 4.2e−07 |
| | | 42/61 (69%) | |
| ig | 258 . . . 313 | 19/59 (32%) | 1.3e−08 |
| | | 42/59 (71%) | |
| ig | 349 . . . 407 | 18/62 (29%) | 2.5e−05 |
| | | 42/62 (68%) | |
| fn3 | 429 . . . 515 | 27/88 (31%) | 3.1e−21 |
| | | 72/88 (82%) | |
| fn3 | 527 . . . 617 | 25/92 (27%) | 1.3e−12 |
| | | 62/92 (67%) | |
| fn3 | 634 . . . 733 | 28/103 (27%) | 1.4e−08 |
| | | 72/103 (70%) | |
| fn3 | 754 . . . 839 | 24/88 (27%) | 4.7e−09 |
| | | 57/88 (65%) | |
| fn3 | 851 . . . 939 | 28/91 (31%) | 1.7e−14 |
| | | 73/91 (80%) | |

### Example 12

The NOV12 clone was analyzed, and the nucleotide and encoded polypeptide sequences are shown in Table 12A.

TABLE 12A
NOV12 Sequence Analysis

| | | |
|---|---|---|
| NOV12a, CG55688-01 | SEQ ID NO: 149 | 1887 bp |
| DNA Sequence | ORF Start: ATG at 81 | ORF Stop: TAA at 1224 |

GCGCACGGCCTGTCCGCTGCACACCAGCTTGTTGGCGTCTTCGTCGCCGCGCTCGCCCCGGGCTACTCCTGCG
CGCCACAATGAGCTCCCGCATCGCCAGGGCGCTCGCCTTAGTCGTCACCCTTCTCCACTTGACCAGGCTGGCG
CTCTCCACCTGCCCCGCTGCCTGCCACTGCCCCCTGGAGGCGCCCAAGTGCGCGCCGGGAGTCGGGCTGGTCC
GGGACGGCTGCGGCTGCTGTAAGGTCTGCGCCAAGCAGCTCAACGAGGACTGCAGCAAAACGCAGCCCTGCGA
CCACACCAAGGGGCTGGAATGCAACTTCGGCGCCAGCTCCACCGCTCTGAAGGGGATCTGCAGAGCTCAGTCA
GAGGGCAGACCCTGTGAATATAACTCCAGAATCTACCAAAACGGGGAAAGTTTCCAGCCCAACTGTAAACATC
AGTGCACATGTATTGATGGCGCCGTGGGCTGCATTCCTCTGTGTCCCCAAGAACTATCTCTCCCCAACTTGGG
CTGTCCCAACCCTCGGCTGGTCAAAGTTACCGGGCAGTGCTGCGAGGAGTGGGTCTGTGACGAGGATAGTATC
AAGGACCCCATGGAGGACCAGGACGGCCTCCTTGGTAAGGAGCTGGGATTCGATGCCTCCGAGGTGGAGTTGA
CGAGAAACAATGAATTGATTGCAGTTGCAAAAGGCAGCTCACTGAAGCGGATCCCTGTTTTTGGAATGGAGCC
TCGCATCCGATACAACCCTTTACAAGGCCAGAAATGTATTGTTCAAACAACTTCATGGTCCCAGTGCTCAAAG
ACCTGTGGAACTGGTATCTCCACACGAGTTACCAATGACAACCCTGAGTGCCGCCTTGTGAAAGAAACCCGCA
TTTGTGAGGTGCGGCCTTGTGGACAGCCAGTGTACAGCAGCCTGAAAAAGGGCAAGAAATGCAGCAAGACCAA
GAAATCCCCCGAACCAGTCAGGTTTACTTACGCTGGATGTTTGAGTGTGAAGAAATACCGGCCCAAGTACTGC
GGTTCCTGCGTGGACGGCCGATGCTGCACGCCCCAGCTGACCAGGACTGTGAAGATGCGGTTCGCCTGCGAAG
ATGGGGAGACATTTTCCAAGAACGTCATGATGATCCAGTCCTGCAAATGCAACTACAACTGCCCGCATGCCAA
TGAAGCAGCGTTTCCCTTCTACAGGCTGTTCAATGACATTCACAAATTTAGGGACTAAATGCTACCTGGGTTT
CCAGGGCACACCTAGACAAACAAGGGAGAAGAGTGTCAGAATCAGAATCATGGAGAAAATGGGCGGGGGTGGT
GTGGGTGATGGGACTCATTGTAGAAAGGAAGCCTTGCTCATTCTTGAGGAGCATTAAGGTATTTCGAAACTGC
CAAGGGTGCTGGTGCGGATGGACACTAATGCAGCCACGATTGGAGAATACTTTGCTTCATAGTATTGGAGCAC
ATGTTACTGCTTCATTTTGGAGCTTGTGGAGTTGATGACTTTCTGTTTTCTGTTTGTAAATTATTTGCTAAGC
ATATTTTCTCTAGGCTTTTTTCCTTTTGGGGTTCTACAGTCGTAAAAGAGATAATAAGATTAGTTGGACAGTT
TAAAGCTTTTATTCGTCCTTTGACAAAAGTAAATGGGAGGGCATTCCATCCCTTCCTGAAGGGGGACACTCCA
TGAGTGTCTGTGAGAGGCAGCTATCTGCACTCTAAACTGCAAACAGAAATCAGGTGTTTTAAGACTGAATGTT
TTATTTATCAAAATGTAGCTTTTGGGGAGGGAGGGGAAATGTAATACTGGAATAATTTGTAAATGATTTTAAT
TTTATATTCAGTGAAAAGATTTTATTTATGGAATTAACCATTTAATAAAGAAATATTTACCT

| | | | |
|---|---|---|---|
| NOV12a, CG55688-01 | | | |
| Protein Sequence | SEQ ID NO: 150 | 381 aa | MW at 42069.1kD |

MSSRIARALALVVTLLHLTRLALSTCPAACHCPLEAPKCAPGVGLVRDGCGCCKVCAKQLNEDCSKTQPCDHT
KGLECNFGASSTALKGICRAQSEGRPCEYNSRIYQNGESFQPNCKHQCTCIDGAVGCIPLCPQELSLPNLGCP
NPRLVKVTGQCCEEWVCDEDSIKDPMEDQDGLLGKELGFDASEVELTRNNELIAVGKGSSLKRIPVFGMEPRI
RYNPLQGQKCIVQTTSWSQCSKTCGTGISTRVTNDNPECRLVKETRICEVRPCGQPVYSSLKKGKKCSKTKKS
PEPVRFTYAGCLSVKKYRPKYCGSCVDGRCCTPQLTRTVKMRFRCEDGETFSKNVMMIQSCKCNYNCPHANEA
AFPFYRLFNDIHKFRD

| | | |
|---|---|---|
| NOV12b, 254087906 | SEQ ID NO: 151 | 1158 bp |
| DNA Sequence | ORF Start: at 1 | ORF Stop: end of sequence |

AGATCTACCATGAGCTCCCGCATCGCCAGGGCGCTCGCCTTAGTCGTCACCCTTCTCCACTTGACCAGGCTGG
CGCTCTCCACCTGCCCCGCTGCCTGCCACTGCCCCCTGGAGGCGCCCAAGTGCGCGCCGGGAGTCGCGCTGGT
CCGGGACGGCTGCGGCTGCTGTAAGGTCTGCGCCAAGCAGCTCAACGAGGACTGCAGCAAAACGCAGCCCTGC
GACCACACCAAGGGGCTGGAATGCAACTTCGGCGCCAGCTCCACCGCTCTGAAGGGGATCTGCAGAGCTCAGT
CAGAGGGCAGACCCTGTGAATATAACTCCAGAATCTACCAAAACGGGGAAAGTTTCCAGCCCAACTGTAAACA
TCAGTGCACATGTATTGATGGCGCCGTGGGCTGCATTCCTCTGTGTCCCCAAGAACTATCTCTCCCCAACTTG
GGCTGTCCCAACCCTCGGCTGGTCAAAGTTACCGGGCAGTGCTGCGAGGAGTGGGTCTGTGACGAGGATAGTA
TCAAGGACCCCATGGAGGACCAGGACGGCCTCCTTGGCAAGGAGCTGGGATTCGATGCCTCCGAGGTGGAGTT
GACGAGAAACAATGAATTGATTGCAGTTGGAAAAGGCAGCTCACTGAAGCGGCTCCCTGTTTTTGGAATGGAG
CCTCGCATCCTATACAACCCTTTACAAGGCCAGAAATGTATTGTTCAAACAACTTCATGGTCCCAGTGCTCAA
AGACCTGTGGAACTGGTATCTCCACACGAGTTACCAATGACAACCCTGAGTGCCGCCTTGTGAAAGAAACCCG
GATTTGTGAGGTGCGGCCTTGTGGACAGCCAGTGTACAGCAGCCTGAAAAAGGGCAAGAAATGCAGCAAGACC
AAGAAATCCCCCGAACCAGTCAGGTTTACTTACGCTGGATGTTTGAGTGTGAAGAAATACCGGCCCAAGTACT
GCGGTTCCTGCGTGGACGGCCGATGCTGCACGCCCCAGCTGACCAGGACTGTGAAGATGCGGTTCCGCTGCGA
AGATGGGGAGACATTTTCCAAGAACGTCATGATGATCCAGTCCTGCAAATGCAACTACAACTGCCCGCATGCC
AATGAAGCAGCGTTTCCCTTCTACAGGCTGTTCAATGACATTCACAAATTTAGGGACCTCGAG

| | | | |
|---|---|---|---|
| NOV12b, 254087906 | | | |
| Protein Sequence | SEQ ID NO: 152 | 386 aa | MW at 42612.7kD |

RSTMSSRIARALALVVTLLHLTRLALSTCPAACHCPLEAPKCAPGVGLVRDGCGCCKVCAKQLNEDCSKTQPC
DHTKGLECNFGASSTALKGICRAQSEGRPCEYNSRIYQNGESFQPNCKHQCTCIDGAVGCIPLCPQELSLPNL
GCPNPRLVKVTGQCCEEWVCDEDSIKDPMEDQDGLLGKELGFDASEVELTRNNELIAVGKGSSLKRLPVFGME
PRILYNPLQGQKCIVQTTSWSQCSKTCGTGISTRVTNDNPECRLVKETRICEVRPCGQPVYSSLKKGKKCSKT
KKSPEPVRFTYAGCLSVKKYRPKYCGSCVDGRCCTPQLTRTVKMRFRCEDGETFSKNVMMIQSCKCNYNCPHA
NEAAFPFYRLFNDIHKFRDLE

| | | |
|---|---|---|
| NOV12c, 259278648 | SEQ ID NO: 153 | 204 bp |
| DNA Sequence | ORF Start: at 1 | ORF Stop: end of sequence |

AGATCTTGTGAATATAACTCCAGAATCTACCAAAACGGGGAAAGTTTCCAGCCCAACTGTAAACATCAGTGCA
CATGTATTGATGGCGCCGTGGGCTGCATTCCTCTGTCTCCCCAAGAACTATCTCTCCCCAACTTGGGCTGTCC
CAACCCTCGGCTGGTCAAAGTTACCGGGCAGTGCTGCGAGGAGTGGGTCTGTCTCGAG

| | | | |
|---|---|---|---|
| NOV12c, 259278648 | | | |
| Protein Sequence | SEQ ID NO: 154 | 68 aa | MW at 7576.6kD |

RSCEYNSRIYQNGESFQPNCKHQCTCIDGAVGCIPLCPQELSLPNLGCPNPRLVKVTGQCCEEWVCLE

| | | |
|---|---|---|
| NOV12d, 259280032 | SEQ ID NO: 155 | 228 bp |
| DNA Sequence | ORF Start: at 1 | ORF Stop: end of sequence |

AGATCTTGCCCCGCTGCCTGCCACTGCCCCCTGGAGGCGCCCAAGTGCGCGCCGGGAGTCGGGCTGGTCCGGG
ACGGCTGCGGCTGCTGTAAGGTCTGCGCCAAGCAGCTCAACGAGGACTGCAGCAAAACGCAGCCCTGCGACCA
CACCAAGGGGCTGGAATGGAACTTCGGCGCCAGCTCCACCGCTCTGAAGGGGATCTGCAGAGCTCAGTCAGAG
GGCCTCGAG

| | | | |
|---|---|---|---|
| NOV12d, 259280032 | | | |
| Protein Sequence | SEQ ID NO: 156 | 76 aa | MW at 7856.9kD |

RSCPAACHCPLEAPKCAPGVGLVRDGCGCCKVCAKQLNEDCSKTQPCDHTKGLECNFGASSTALKGICRAQSE
GLE

| | | |
|---|---|---|
| NOV12e, 254756530 | SEQ ID NO: 157 | 228 bp |
| DNA Sequence | ORF Start: at 1 | ORF Stop: end of sequence |

AGATCTTGCCCCGCTGCCTGCCACTGCCCCCTGGAGGCGCCCAAGTGCGCGCCGGGAGTCGGGCTGGTCCGGG
ACGGCTGCGGCTGCTGTAAGGTCTGCGCCAAGCAGCTCAACGAGGACTGCAGCAAAACGCAGCCCTGCGACCA
CACCAAGGGGCTGGAATGCAACTTCGGCGCCAGCTCCACCGCTCTGAAGGGGATCTGCAGAGCTCAGTCAGAG
GGCCTCGAG

| | | | |
|---|---|---|---|
| NOV12e, 254756530 | | | |
| Protein Sequence | SEQ ID NO: 158 | 76 aa | MW at 7856.9kD |

RSCPAACHCPLEAPKCAPGVGLVRDGCGCCKVCAKQLNEDCSKTQPCDHTKGLECNFGASSTALKGICRAQSE
GLE

| | | |
|---|---|---|
| NOV12f, 229509618 | SEQ ID NO: 159 | 1228 bp |
| DNA Sequence | ORF Start: at 3 | ORF Stop: at 1227 |

TGTACAAGAAAGCTGGGTCGCCGCGCCCACCCTTCACCACCATGAGCTCCCGCATCGCCAGGGCGCTCGCCTT
AGTCGTCACCCTTCTCCACTTGACCAGGCTGGCGCTCTCCACCTGCCCCGCTGCCTGCCACTGCCCCCTGGAG
GCGCCCAAGTGCGCGCCGGGAGTCGGGCTGGTCCGGGACGGCTGCGGCTCCTGTAAGGTCTGCGCCAAGCAGC
TCAACGAGGACTGCAGCAAAACGCAGCCCTGCGACCACACCAAGGGGCTGGAATGCAACTTCGGCGCCAGCTC
CACCGCTCTGAAGGGGATCTGCAGAGCTCAGTCAGAGGGCAGACCCTGTGAATATAACTCCAGAATCTACCAA
AACGGGGAAAGTTTCCAGCCCAACTGTAAACATCAGTGCACATGTATTGATGGCGCCGTGGGCTGCATTCCTC
TGTGTCCCCAAGAACTATCTCTCCCCAACTTGGGCTGTCCCAACCCTCGGCTGGTCAAAGTTACCGGGCAGTG
CTGCGAGGAGTGGGTCTGTGACGAGGATAGTATCAAGGACCCCATGGAGGACCAGGACGGCCTCCTTGGCAAG
GAGCTGGGATTCGATGCCTCCGAGGTGGAGTTGACGAGAAACAATGAATTGATTGCAGTTGGAAAAGGCAGCT
CACTGAAGCGGCTCCCTGTTTTTGGAATGGAGCCTCGCATCCTATACAACCCTTTACAAGGCCAGAAATGTAT
TGTTCAAACAACTTCATGGTCCCAGTGCTAAACGACCTGTGGAACTGGTATCTCCACACGAGTTACCAATGAC
AACCCTGAGTGCCGCCTTGTGAAAGAAACCCGGATTTGTGAGGTGCGGCCTTGTGGACAGCCAGTGTACAGCA
GCCTGAAAAAGGGCAAGAAATGCAGCAAGACCAAGAAATCCCCCGAACCAGTCAGGTTTACTTACGCTGGATG
TTTGAGTGTGAAGAAATACCGGCCCAAGTACTGCGGTTCCTGCGTGGACGGCCGATGCTGCACGCCCCAGCTG
ACCAGGACTGTGAAGATGCGGTTCCGCTGCGAAGATGGGGAGACATTTTCCAAGAACGTCATGATGATCCAGT
CCTGCAAATGCAACTACAACTGCCCGCATGCCAATGAAGCAGCGTTTCCCTTCTACAGGCTGTTCAATGACAT
TCACAAATTTAGGGACAAGGGTGGGCGCGCCCTTTCTCGAGTACGGCGGCCGCGGAGCCT

| | | | |
|---|---|---|---|
| NOV12f, 229509618 | | | |
| Protein Sequence | SEQ ID NO: 160 | 408 aa | MW at 45009.5kD |

YKKAGSARPPFTTMSSRIARALALVVTLLHLTRLALSTCPAACHCPLEAPKCAPGVGLVRDGCGCCKVCAKQL
NEDCSKTQPCDHTKGLECNFGASSTALKGICRAQSEGRPCEYNSRIYQNGESFQPNCKHQCTCIDGAVGCIPL
CPQELSLPNLGCPNPRLVKVTGQCCEEWVCDEDSIKDPMEDQDGLLGKELGFDASEVELTRNNELIAVGKGSS
LKRLPVFGMEPRILYNPLQGQKCIVQTTSWSQCSKTCGTGISTRVTNDNPECRLVKETRICEVRPCGQPVYSS
LKKGKKCSKTKKSPEPVRFTYAGCLSVKKYRPKYCGSCVDGRCCTPQLTRTVKMRFRCEDGETFSKNVMMIQS
CKCNYNCPHANEAAFPFYRLFNDIHKFRDKGGRALSRVRRPRS

| | | |
|---|---|---|
| NOV12g, 229509658 | SEQ ID NO: 161 | 1111 bp |
| DNA Sequence | ORF Start: at 2 | ORF Stop: end of sequence |

AGGCTCCGCGGCCGCCCCCTTCACCACCTGCCCCGCTGCCTGCCACTGCCCCCTGGAGGCGCCCAAGTGCGCG
CCGGGAGTCGGGCTGGTCCGGGACGGCTGCGGCTGCTGTAAGGTCTGCGCCAAGCAGCTCAACCAGGACTGCA
GCAAAACGCACCCCTGCGACCACACCAAGGGGCTGGAATGCAACTTCGGCGCCAGCTCCACCGCTCTGAAGGG
GATCTGCAGAGCTCAGTCAGAGGGCAGACCCTGTGAATATAACTCCAGAATCTACCAAAACGGGGAAAGTTTC
CAGCCCAACTGTAAACATCAGTGCACATGTATTGATGGCGCCGTGGGCTGCATTCCTCTGTGTCCCCAAGAAC
TATCTCTCCCCAACTTGGGCTGTCCCAACCCTCGGCTGGTCAAAGTTACCGGGCAGTGCTGCGAGGAGTGGGT
CTGTGACGAGGATAGTATCAAGGACCCCATGGAGGACCAGGACGGCCTCCTTGGCAAGGAGCTGGGATTCGAT
GCCTCCGAGGTGGAGTTAACGAGAAACAATGAATTGATTGCAGTTGGAAAAGGCAGCTCACTGAAGCGGCTCC
CTGTTTTTGGAATGGAGCCTCGCATCCTATACAACCCTTTACAAGGCCAGAAATGTATTGTTCAAACAACTTC
ATGGTCCCAGTGCTCAAAGACCTGTGGAACTGGTATCTCCACACGAGTTACCAATGACAACCCTGAGTGCCGC
CTTGTGAAAGAAACCCGCATTTGTGAGGTGCGGCCTTGTGGACAGCCAGTGTACAGCAGCCTGAAAAAGGGCA
AGAAATGCAGCAAGACCAAGAAATCCCCCGAACCAGTCAGGTTTACTTACGCTGGATGTTTGAGTGTGAAGAA
ATACCGGCCCAAGTACTGCGGTTCCTGCGTGGACGCCCGATGCTGCACGCCCCAGCTGACCAGGACTGTGAAG
ATGCGGTTCCGCTGCGAAGATGGGGAGACATTTTCCAAGAACGTCATGATGATCCAGTCCTGCAAATGCAACT
ACAACTGCCCGCATGCCAATGAAGCAGCGTTTCCCTTCTACAGGCTGTTCAATGACATTCACAAATTTAGGGA
CAAGGGTGGGCGCGCC

| | | | |
|---|---|---|---|
| NOV12g, 229509658 | | | |
| Protein Sequence | SEQ ID NO: 162 | 370 aa | MW at 40610.3kD |

GSAAAPFTTCPAACHCPLEAPKCAPGVGLVRDGCGCCKVCAKQLNEDCSKTQPCDHTKGLECNFGASSTALKG
ICRAQSEGRPCEYNSRIYQNGESFQPNCKHQCTCIDGAVGCIPLCPQELSLPNLGCPNPRLVKVTGQCCEEWV
CDEDSIKDPMEDQDGLLGKELGFDASEVELTRNNELIAVGKGSSLKRLPVFGMEPRILYNPLQGQKCIVQTTS
WSQCSKTCGTGISTRVTNDNPECRLVKETRICEVRPCGQPVYSSLKKGKKCSKTKKSPEPVRFTYAGCLSVKK
YRPKYCGSCVDGRCCTPQLTRTVKMRFRCEDGETFSKNVMMIQSCKCNYNCPHANEAAFPFYRLFNDIHKFRD
KGGRA

| | | |
|---|---|---|
| NOV12h, CG55688-02 | SEQ ID NO: 163 | 1068 bp |
| DNA Sequence | ORF Start: at 1 | ORF Stop: end of sequence |

ACCTGCCCCGCTGCCTGCCACTGCCCCCTGGAGGCGCCCAAGTGCGCGCCGGGAGTCGGGCTGGTCCGGGACG
GCTGCGGCTGCTGTAAGGTCTGCGCCAAGCAGCTCAACGAGGACTGCAGCAAAACGCAGCCCTGCGACCACAC
CAAGGGGCTGGAATGCAACTTCGGCGCCAGCTCCACCGCTCTGAAGGGGATCTGCAGAGCTCAGTCAGAGGGC
AGACCCTGTGAATATAACTCCAGAATCTACCAAAACGGGGAAAGTTTCCAGCCCAACTGTAAACATCAGTGCA
CATGTATTGATGGCGCCGTGGGCTGCATTCCTCTGTGTCCCCAAGAACTATCTCTCCCCAACTTGGGCTGTCC
CAACCCTCGGCTGGTCAAAGTTACCGGGCAGTGCTGCGAGGAGTGGGTCTGTGACGAGGATAGTATCAAGGAC
CCCATGGAGGACCAGGACGGCCTCCTTGGCAAGGAGCTGGGATTCGATGCCTCCGAGGTGCAGTTGACGAGAA
ACAATGAATTGATTGCAGTTGGAAAAGGCAGCTCACTGAAGCGGCTCCCTGTTTTTGGAATGGAGCCTCGCAT
CCTATACAACCCTTTACAAGGCCAGAAATGTATTGTTCAAACAACTTCATGGTCCCAGTGCTCAAAGACCTGT
GGAACTGGTATCTCCACACGAGTTACCAATGACAACCCTGAGTGCCGCCTTGTGAAAGAAACCCGGATTTGTG
AGGTGCGGCCTTGTGGACAGCCAGTGTACAGCAGCCTGAAAAAGGGCAAGAAATGCAGCAAGACCAAGAAATC
CCCCGAACCAGTCAGGTTTACTTACGCTGGATGTTTGAGTGTGAAGAAATACCGGCCCAAGTACTGCGGTTCC
TGCGTGGACGGCCGATGCTGCACGCCCCAGCTGACCAGGACTGTGAAGATGCGGTTCCGCTGCGAAGATGGGG
AGACATTTTCCAAGAACGTCATGATGATCCAGTCCTGCAAATGCAACTACAACTGCCCGCACGCCAATGAAGC
AGCGTTTCCCTTCTACAGGCTGTTCAATGACATTCACAAATTTAGG

| | | | |
|---|---|---|---|
| NOV12h, CG55688-02 | | | |
| Protein Sequence | SEQ ID NO: 164 | 356 aa | MW at 39322.9kD |

TCPAACHCPLEAPKCAPGVGLVRDGCGCCKVCAKQLNEDCSKTQPCDHTKGLECNFGASSTALKGICRAQSEG
RPCEYNSRIYQNGESFQPNCKHQCTCIDGAVGCIPLCPQELSLPNLGCPNPRLVKVTGQCCEEWVCDEDSIKD
PMEDQDGLLGKELGFDASEVELTRNNELIAVGKGSSLKRLPVFGMEPRILYNPLQGQKCIVQTTSWSQCSKTC
GTGISTRVTNDNPECRLVKETRICEVRPCGQPVYSSLKKGKKCSKTKKSPEPVRFTYAGCLSVKKYRPKYCGS
CVDGRCCTPQLTRTVKMRFRCEDGETFSKNVMMIQSCKCNYNCPHANEAAFPFYRLFNDINKFR

| | | |
|---|---|---|
| NOV12i, CG55688-03 | SEQ ID NO: 165 | 1198 bp |
| DNA Sequence | ORF Start: at 2 | ORF Stop: end of sequence |

GTACAAAAAAGCAGGCTCCGCGGCCGCCCCCTTCACCACCATGAGCTCCCGCATCGCCAGGGCGCTCGCCTTA
GTCGTCACCCTTCTCCACTTGACCAGGCTGGCGCTCTCCACCTGCCCCGCTGCCTGCCACTGCCCCCTGGAGG
CGCCCAAGTGCGCGCCGGGAGTCGGGCTGGTCCGGGACGGCTGCGCCTGCTGTAAGGTCTGCGCCAAGCAGCT
CAACGAGGACTGCAGCAAAACGCAGCCCTGCGACCACACCAAGGGGCTGGAATGCAACTTCGGCGCCAGCTCC
ACCGCTCTGAAGGGGATCTGCAGAGCTCAGTCAGAGGGCAGACCCTGTGAATATAACTCCAGAATCTACCAAA
ACGGGGAAAGTTTCCAGCCCAACTGTAAACATCAGTGCACATGTATTGATGGCGCCGTGGGCTGCATTCCTCT
GTGTCCCCAAGAACTATCTCTCCCCAACTTGGGCTGTCCCAACCCTCGGCTGGTCAAAGTTACCGGGCAGTGC
TGCGAGGAGTGGGTCTGTGACGAGGATAGTATCAAGGACCCCATGGAGGACCAGGACGGCCTCCTTGGCAAGG
AGCTGGGATTCGATGCCTCCGAGGTGGAGTTGACGAGAAACAATGAATTGATTGCAGTTGGAAAAGGCAGCTC
ACTGAAGCGGCTCCCTGTTTTTGGAATGGAGCCTCGCATCCTATACAACCCTTTACAAGGCCAGAAATGTATT
GTTCAAACAACTTCATGGTCCCAGTGCTCAAAGACCTGTGGAACTGGTATCTCCACACGAGTTACCAATGACA
ACCCTGAGTGCCGCCTTGTGAAAGAAACCCGGATTTGTGAGGTGCGGCCTTGTGGACAGCCAGTGTACAGCAG
CCTGAAAAAGGGCAAGAAATGCAGCAAGACCAAGAAATCCCCCGAACCAGTCAGGTTTACTTACGCTGGATGT
TTGAGTGTGAAGAAATACCGGCCCAAGTACTGCGGTTCCTGCGTGGACGGCCGATGCTGCACGCCCCAGCTGA
CCAGGACTGTGAAGATGCGGTTCCGCTGCGAAGATGGGGAGACATTTTCCAAGAACGTCATGATGATCCAGTC
CTGCAAATGCAACTACAACTGCCCGCATGCCAATGAAGCAGCGTTTCCCTTCTACAGGCTGTTCAATGACATT
CACAAATTTAGGGACAAGGGTGGGCGCGCC

| | | | |
|---|---|---|---|
| NOV12i, CG55688-03 | | | |
| Protein Sequence | SEQ ID NO: 166 | 399 aa | MW at 43790.1kD |

YKKAGSAAAPFTTMSSRIARALALVVTLLHLTRLALSTCPAACHCPLEAPKCAPGVGLVRDGCGCCKVCAKQL
NEDCSKTQPCDHTKGLECNFGASSTALKGICRAQSEGRPCEYNSRIYQNGESFQPNCKHQCTCIDGAVGCIPL
CPQELSLPNLGCPNPRLVKVTGQCCEEWVCDEDSIKDPMEDQDGLLGKELGFDASEVELTRNNELIAVGKGSS
LKRLPVFGMEPRILYNPLQGQKCIVQTTSWSQCSKTCGTGISTRVTNDNPECRLVKETRICEVRPCGQPVYSS
LKKGKKCSKTKKSPEPVRFTYAGCLSVKKYRPKYCGSCVDGRCCTPQLTRTVKMRFRCEDGETFSKNVMMIQS
CKCNYNCPHANEAAFPFYRLFNDIHKFRDKGGRA

| | | |
|---|---|---|
| NOV12j, CG55688-04 | SEQ ID NO: 167 | 1111 bp |
| DNA Sequence | ORF Start: at 2 | ORF Stop: end of sequence |

AGGCTCCGCGGCCGCCCCCTTCACCACCTGCCCCGCTGCCTGCCACTGCCCCCTGGAGGCGCCCAAGTGCGCG
CCGGGAGTCGGGCTGGTCCGGGACGGCTGCGGCTGCTGTAAGGTCTGCGCCAAGCAGCTCAACGAGGACTGCA
GCAAAACGCAGCCCTGCGACCACACCAAGGGGCTGGAATGCAACTTCGGCGCCAGCTCCACCGCTCTGAAGGG
GATCTGCAGAGCTCAGTCAGAGGGCAGACCCTGTGAATATAACTCCAGAATCTACCAAAACGGGGAAAGTTTC
CAGCCCAACTGTAAACATCAGTGCACATGTATTGATGGCGCCGTGGGCTGCATTCCTCTGTGTCCCCAAGAAC
TATCTCTCCCCAACTTGGGCTGTCCCAACCCTCGGCTGGTCAAAGTTACCGGGCAGTGCTGCGAGGAGTGGGT
CTGTGACGAGGATAGTATCAAGGACCCCATGGAGGACCAGGACGGCCTCCTTGGCAAGGAGCTGGGATTCGAT
GCCTCCGAGGTGGAGTTAACGAGAAACAATGAATTGATTGCAGTTGGAAAAGGCAGCTCACTGAAGCGGCTCC
CTGTTTTTGGAATCGAGCCTCGCATCCTATACAACCCTTTACAAGGCCAGAAATGTATTGTTCAAACAACTTC
ATGGTCCCAGTGCTCAAAGACCTGTGGAACTGGTATCTCCACACGAGTTACCAATGACAACCCTGAGTGCCGC
CTTGTGAAAGAAACCCGGATTTGTGAGGTGCGGCCTTGTGGACAGCCAGTGTACAGCAGCCTGAAAAAGGGCA
AGAAATGCAGCAAGACCAAGAAATCCCCCGAACCAGTCAGGTTTACTTACGCTGGATGTTTGAGTGTGAAGAA
ATACCGGCCCAAGTACTGCGGTTCCTGCGTGGACGGCCGATGCTGCACGCCCCAGCTGACCAGGACTGTGAAG
ATGCGGTTCCGCTGCGAAGATGGGGAGACATTTTCCAAGAACGTCATGATGATCCAGTCCTGCAAATGCAACT
ACAACTGCCCGCATGCCAATGAAGCAGCGTTTCCCTTCTACAGGCTGTTCAATGACATTCACAAATTTAGGGA
CAAGGGTGGGCGCGCC

| | | | |
|---|---|---|---|
| NOV12j, CG55688-04 | | | |
| Protein Sequence | SEQ ID NO: 168 | 370 aa | MW at 406 10.3kD |

GSAAAPFTTCPAACHCPLEAPKCAPGVGLVRDGCGCCKVCAKQLNEDCSKTQPCDHTKGLECNFGASSTALKG
ICRAQSEGRPCEYNSRIYQNGESFQPNCKHQCTCIDGAVGCIPLCPQELSLPNLGCPNPRLVKVTGQCCEEWV
CDEDSIKDPMEDQDGLLGKELGFDASEVELTRNNELIAVGKGSSLKRLPVFGMEPRILYNPLQGQKCIVQTTS
WSQCSKTCGTGISTRVTNDNPECRLVKETRICEVRPCGQPVYSSLKKGKKCSKTKKSPEPVRFTYAGCLSVKK
YRPKYCGSCVDGRCCTPQLTRTVKMRFRCEDGETFSKNVMMIQSCKCNYNCPHANEAAFPFYRLFNDIHKFRD
KGGRA

| | | |
|---|---|---|
| NOV12k, CG55688-05 | SEQ ID NO: 169 | 1174 bp |
| DNA Sequence | ORF Start: ATG at 23 | ORF Stop: at 1166 |

GAATTCGCCCTTCACCAGATCTATGAGCTCCCGCATCGCCAGGGCGCTCGCCTTAGTCGTCACCCTTCTCCAC
TTGACCAGGCTGGCGCTCTCCACCTGCCCCGCTGCCTGCCACTGCCCCCTGGAGGCGCCCAAGTGCGCGCCGG
GAGTCGGGCTGGTCCGGGACGGCTGCGGCTGCTGTAAGGTCTGCGCCAAGCAGCTCAACGAGGACTGCAGCAA
AACGCAGCCCTGCGACCACACCAAGGGGCTGGAATGCAACTTCGGCGCCAGCTCCACCGCTCTGAAGGGGATC
TGCAGAGCTCAGTCAGAGGGCAGACCCTGTGAATATAACTCCAGAATCTACCAAAACGGGGAAAGTTTCCAGC
CCAACTGTAAACATCAGTGCACATGTATTGATGGCGCCGTGGGCTGCATTCCTCTGTGTCCCCAAGAACTATC
TCTCCCCAACTTGGGCTGTCCCAACCCTCGGCTGGTCAAAGTTACCGGGCAGTGCTGCGAGGAGTGGGTCTGT
GACGAGGATAGTATCAAGGACCCCATGGAGGACCAGGACGGCCTCCTTGGCAAGGAGCTGGGATTCGATGCCT
CCGAGGTGGAGTTGACGAGAAACAATGAATTGATTGCAGTTGGAAAAGGCAGCTCACTGAAGCGGCTCCCTGT
TTTTGGAATGGAGCCTCGCATCCTATACAACCCTTTACAAGGCCAGAAATGTATTGTTCAAACAACTTCATGG
TCCCAGTGCTCAAAGACCTGTGGAACTGGTATCTCCACACGAGTTACCAATGACAACCCTGAGTGCCGCCTTG
TGAAAGAAACCCGGATTTGTGAGGTGCGGCCTTGTGGACAGCCAGTGTACAGCAGCCTGAAAAAGGGCAAGAA
ATGCAGCAAGACCAAGAAATCCCCCGAACCAGTCAGGTTTACTTACGCTGGATGTTTGAGTGTGAAGAAATAC
CGGCCCAAGTACTGCGGTTCCTGCGTGGACGGCCGATGCTGCACGCCCCAGCTGACCAGGACTGTGAAGATGC
GGTTCCGCTGCGAAGATGGGGAGACATTTTCCAAGAACGTCATGATGATCCAGTCCTGCAAATGCAACTACAA
CTGCCCGCATGCCAATGAAGCAGCGTTTCCCTTCTACAGGCTGTTCAATGACATTCACAAATTTAGGGACCTC
GAGGGC

| | | | |
|---|---|---|---|
| NOV12k, CG55688-05 | | | |
| Protein Sequence | SEQ ID NO: 170 | 381 aa | MW at 42026.1kD |

MSSRIARALALVVTLLHLTRLALSTCPAACHCPLEAPKCAPGVGLVRDGCGCCKVCAKQLNEDCSKTQPCDHT
KGLECNFGASSTALKGICRAQSEGRPCEYNSRIYQINGESFQPNCKHQCTCIDGAVGCIPLCPQELSLPNLGCP
NPRLVKVTGQCCEEWVCDEDSIKDPMEDQDGLLGKELGFDASEVELTRNNELIAVGKGSSLKRLPVFGMEPRI
LYNPLQGQKCIVQTTSWSQCSKTCGTGISTRVTNDNPECRLVKETRICEVRPCGQPVYSSLKKGKKCSKTKKS
PEPVRFTYAGCLSVKKYRPKYCGSCVDGRCCTPQLTRTVKMRFRCEDGETFSKNVMMIQSCKCNYNCPHANEA
AFPFYRLFNDIHKFRD

| | | |
|---|---|---|
| NOV12l, CG55688-06 | SEQ ID NO: 171 | 1168 bp |
| DNA Sequence | ORF Start: ATG at 14 | ORF Stop: TAG at 1157 |

CACCGGATCCACCATGAGCTCCCGCATCGCCAGGGCGCTCGCCTTAGTCGTCACCCTTCTCCACTTGACCAGG
CTGGCGCTCTCCACCTGCCCCGCTGCCTGCCACTGCCCCCTGGAGGCGCCCAAGTGCGCGCCGGGAGTCGGGC
TGGTCCGGGACGGCTGCGGCTGCTGTAAGGTCTGCGCCAAGCAGCTCAACGAGGACTGCAGCAAAACGCAGCC
CTGCGACCACACCAAGGGGCTGGAATGCAACTTCGGCGCCAGCTCCACCGCTCTGAAGGGGATCTGCAGAGCT
CAGTCAGAGGGCAGACCCTGTGAATATAACTCCAGAATCTACCAAAACGGGGAAAGTTTCCAGCCCAACTGTA
AACATCAGTGCACATGTATTGATGGCGCCGTCGGCTGCATTCCTCTGTGTCCCCAAGAACTATCTCTCCCCAA
CTTGGGCTGTCCCAACCCTCGGCTGGTCAAAGTTACCGGGCAGTGCTGCGAGGAGTGGGTCTGTGACCAGGAT
AGTATCAAGGACCCCATGGAGGACCAGGACGGCCTCCTTGGCAAGGAGCTGGGATTCGATGCCTCCGAGGTGG
AGTTGACGAGAAACAATGAATTGATTGCAGTTGGAAAAGGCAGCTCACTGAAGCGGCTCCCTGTTTTTGGAAT
GGAGCCTCGCATCCTATACAACCCTTTACAAGGCCAGAAATGTATTGTTCAAACAACTTCATGGTCCCAGTGC
TCAAAGACCTGTGGAACTGGTATCTCCACACGAGTTACCAATGACAACCCTGAGTGCCGCCTTGTGAAAGAAA
CCCGGATTTGTGAGGTGCGGCCTTGTGGACAGCCAGTGTACAGCAGCCTGAAAAAGGGCAAGAAATGCAGCAA
GACCAAGAAATCCCCCGAACCAGTCAGGTTTACTTACGCTGGATGTTTGAGTGTGAAGAAATACCGGCCCAAG
TACTGCGGTTCCTGCGTGGACGGCCGATGCTGCACGCCCCAGCTGACCAGGACTGTGAAGATGCGGTTCCGCT
GCGAAGATGGGGAGACATTTTCCAAGAACGTCATGATGATCCAGTCCTGCAAATGCAACTACAACTGCCCGCA
TGCCAATGAAGCAGCGTTTCCCTTCTACAGGCTGTTCAATGACATTCACAAATTTAGGGACTAGGTCGACGGC

| | | | |
|---|---|---|---|
| NOV12l, CG55688-06 | | | |
| Protein Sequence | SEQ ID NO: 172 | 381 aa | MW at 42026.1kD |

MSSRIARALALVVTLLHLTRLALSTCPAACHCPLEAPKCAPGVGLVRDGCGCCKVCAKQLNEDCSKTQPCDHT
KGLECNFGASSTALKGICRAQSEGRPCEYNSRIYQNGESFQPNCKHQCTCIDGAVGCIPLCPQELSLPNLGCP
NPRLVKVTGQCCEEWVCDEDSIKDPMEDQDGLLGKELGFDASEVELTRNNELIAVGKGSSLKRLPVFGMEPRI
LYNPLQGQKCIVQTTSWSQCSKTCGTGISTRVTNDNPECRLVKETRICEVRPCGQPVYSSLKKGKKCSKTKKS
PEPVRFTYAGCLSVKKYRPKYCGSCVDGRCCTPQLTRTVKMRFRCEDGETFSKNVMMIQSCKCNYNCPHANEA
AFPFYRLFNDIHKFRD

| | | |
|---|---|---|
| | SEQ ID NO: 173 | 1887 bp |
| NOV12m, SNP13376428 of | ORF Start: ATG at 81 | ORF Stop: TAA at 1224 |
| CG55688-01, DNA Sequence | SNP Pos: 571 | SNP change: A to G |

GCGCACGGCCTGTCCGCTGCACACCAGCTTGTTGGCGTCTTCGTCGCCGCGCTCGCCCCGGGCTACTCCTGCG
CGCCACAATGAGCTCCCGCATCGCCAGGGCGCTCGCCTTAGTCGTCACCCTTCTCCACTTGACCAGGCTGGCG
CTCTCCACCTGCCCCGCTGCCTGCCACTGCCCCCTGGAGGCGCCCAAGTGCGCGCCGGGAGTCGGGCTGGTCC
GGGACGGCTGCGGCTGCTGTAAGGTCTGCGCCAAGCAGCTCAACGAGGACTGCAGCAAAACGCAGCCCTGCGA
CCACACCAAGGGGCTGGAATGCAACTTCGGCGCCAGCTCCACCGCTCTGAAGGGGATCTGCAGAGCTCAGTCA
GAGGGCAGACCCTGTGAATATAACTCCAGAATCTACCAAAACGGGGAAAGTTTCCAGCCCAACTGTAAACATC
AGTGCACATGTATTGATGGCGCCGTGGGCTGCATTCCTCTGTGTCCCCAAGAACTATCTCTCCCCAACTTGGG
CTGTCCCAACCCTCGGCTGGTCAAAGTTACCGGGCAGTGCTGCGAGGAGTGGGTCTGTGGCGAGGATAGTATC
AAGGACCCCATGGAGGACCAGGACGGCCTCCTTGGTAAGGAGCTGGGATTCGATGCCTCCGAGGTGGAGTTGA
CGAGAAACAATGAATTGATTGCAGTTGGAAAAGGCAGCTCACTGAAGCGGATCCCTGTTTTTGGAATGGAGCC
TCGCATCCGATACAACCCTTTACAAGGCCAGAAATGTATTGTTCAAACAACTTCATGGTCCCAGTGCTCAAAG
ACCTGTGGAACTGGTATCTCCACACGAGTTACCAATGACAACCCTGAGTGCCGCCTTGTGAAAGAAACCCGGA
TTTGTGAGGTGCGGCCTTGTGGACAGCCAGTGTACAGCAGCCTGAAAAAGGGCAAGAAATGCAGCAAGACCAA
GAAATCCCCCGAACCAGTCAGGTTTACTTACGCTGGATGTTTGAGTGTGAAGAAATACCGGCCCAAGTACTGC
GGTTCCTGCGTGGACGGCCGATGCTGCACGCCCCAGCTGACCAGGACTGTGAAGATGCGGTTCCGCTGCGAAG
ATGGGGAGACATTTTCCAAGAACGTCATGATGATCCAGTCCTGCAAATGCAACTACAACTGCCCGCATGCCAA
TGAAGCAGCGTTTCCCTTCTACAGGCTGTTCAATGACATTCACAAATTTAGGGACTAAATGCTACCTGGGTTT
CCAGGGCACACCTAGACAAACAAGGGAGAAGAGTGTCAGAATCAGAATCATGGAGAAAATGGGCGGGGGTGGT
GTGGGTGATGGGACTCATTGTAGAAAGGAAGCCTTGCTCATTCTTGAGGAGCATTAAGGTATTTCGAAACTGC
CAAGGGTGCTGGTGCGGATGGACACTAATGCAGCCACGATTGGAGAATACTTTGCTTCATAGTATTGGAGCAC
ATGTTACTGCTTCATTTTGGAGCTTGTGGAGTTGATGACTTTCTGTTTTCTGTTTGTAAATTATTTGCTAAGC
ATATTTTCTCTAGGCTTTTTTCCTTTTGGGGTTCTACAGTCGTAAAAGAGATAATAAGATTAGTTGGACAGTT
TAAAGCTTTTATTCGTCCTTTGACAAAAGTAAATGGGAGGGCATTCCATCCCTTCCTGAAGGGGGACACTCCA
TGAGTGTCTGTGAGAGGCAGCTATCTGCACTCTAAACTGCAAACAGAAATCAGGTGTTTTAAGACTGAATGTT
TTATTTATCAAAATGTAGCTTTTGGGGAGGGAGGGGAAATGTAATACTGGAATAATTTGTAAATGATTTTAAT
TTTATATTCAGTGAAAAGATTTTATTTATGGAATTAACCATTTAATAAAGAAATATTTACCT

| | | | |
|---|---|---|---|
| NOV12m, SNP13376428 of | SEQ ID NO: 174 | | MW at42011.1kD |
| CG55688-01, Protein Sequence | SNP Pos: 164 | 381 aa | SNP change: Asp to Gly |

MSSRIARALALVVTLLHLTRLALSTCPAACHCPLEAPKCAPGVGLVRDGCGCCKVCAKQLNEDCSKTQPCDHT
KGLECNFGASSTALKGICRAQSEGRPCEYNSRIYQNGESFQPNCKHQCTCIDGAVGCIPLCPQELSLPNLGCP
NPRLVKVTGQCCEEWVCGEDSIKDPMEDQDGLLGKELGFDASEVELTRNNELIAVGKGSSLKRIPVFGMEPRI
RYNPLQGQKCIVQTTSWSQCSKTCGTGISTRVTNDNPECRLVKETRICEVRPCGQPVYSSLKKGKKCSKTKKS
PEPVRFTYAGCLSVKKYRPKYCGSCVDGRCCTPQLTRTVKMRFRCEDGETFSKNVMMIQSCKCNYNCPHANEA
AFPFYRLFNDIHKFRD

A ClustalW comparison of the above protein sequences yields the following sequence alignment shown in Table 12B.

TABLE 12B
Comparison of the NOV12 protein sequences.

| | |
|---|---|
| NOV12a | -------------MSSRIARALALVVTLLHLTRLALSTCPAACHCPLEAPKCAPGVGLVR |
| NOV12b | ----------RSTMSSRIARALALVVTLLHLTRLALSTCPAACHCPLEAPKCAPGVGLVR |
| NOV12c | ------------------------------------RSCEYNSRIYQNGESFQPNCKHQC |
| NOV12d | ------------------------------------RSCPAACHCPLEAPKCAPGVGLVR |
| NOV12e | ------------------------------------RSCPAACHCPLEAPKCAPGVGLVR |
| NOV12f | YKKAGSARPPFTTMSSRIARALALVVTLLHLTRLALSTCPAACHCPLEAPKCAPGVGLVR |
| NOV12g | -----------------------------GSAAAPFTTCPAACHCPLEAPKCAPGVGLVR |
| NOV12h | -------------------------------------TCPAACHCPLEAPKCAPGVGLVR |
| NOV12i | YKKAGSAAAPFTTMSSRIARALALVVTLLHLTRLALSTCPAACHCPLEAPKCAPGVGLVR |
| NOV12j | -----------------------------GSAAAPFTTCPAACHCPLEAPKCAPGVGLVR |
| NOV12k | -------------MSSRIARALALVVTLLHLTRLALSTCPAACHCPLEAPKCAPGVGLVR |
| NOV12l | -------------MSSRIARALALVVTLLHLTRLALSTCPAACHCPLEAPKCAPGVGLVR |
| NOV12a | DGCGCCKVCAKQLNEDCSKTQPCDHTKGLECNFGASSTALKGICRAQSEGRPCEYNSRIY |
| NOV12b | DGCGCCKVCAKQLNEDCSKTQPCDHTKGLECNFGASSTALKGICRAQSEGRPCEYNSRIY |
| NOV12c | TCIDGAVGCIPLCPQELSLPNLGCPNPRLVKVTGQCCEEWVCLE---------------- |
| NOV12d | DGCGCCKVCAKQLNEDCSKTQPCDHTKGLECNFGASSTALKGICRAQSEGLE-------- |
| NOV12e | DGCGCCKVCAKQLNEDCSKTQPCDHTKGLECNFGASSTALKGICRAQSEGLE-------- |
| NOV12f | DGCGCCKVCAKQLNEDCSKTQPCDHTKGLECNFGASSTALKGICRAQSEGRPCEYNSRIY |
| NOV12g | DGCGCCKVCAKQLNEDCSKTQPCDHTKGLECNFGASSTALKGICRAQSEGRPCEYNSRIY |
| NOV12h | DGCGCCKVCAKQLNEDCSKTQPCDHTKGLECNFGASSTALKGICRAQSEGRPCEYNSRIY |
| NOV12i | DGCGCCKVCAKQLNEDCSKTQPCDHTKGLECNFGASSTALKGICRAQSEGRPCEYNSRIY |
| NOV12j | DGCGCCKVCAKQLNEDCSKTQPCDHTKGLECNFGASSTALKGICRAQSEGRPCEYNSRIY |
| NOV12k | DGCGCCKVCAKQLNEDCSKTQPCDHTKGLECNFGASSTALKGICRAQSEGRPCEYNSRIY |
| NOV12l | DGCGCCKVCAKQLNEDCSKTQPCDHTKGLECNFGASSTALKGICRAQSEGRPCEYNSRIY |
| NOV12a | QNGESFQPNCKHQCTCIDGAVGCIPLCPQELSLPNLGCPNPRLVKVTGQCCEEWVCDEDS |
| NOV12b | QNGESFQPNCKHQCTCIDGAVGCIPLCPQELSLPNLGCPNPRLVKVTGQCCEEWVCDEDS |
| NOV12c | ------------------------------------------------------------ |
| NOV12d | ------------------------------------------------------------ |
| NOV12e | ------------------------------------------------------------ |
| NOV12f | QNGESFQPNCKHQCTCIDGAVGCIPLCPQELSLPNLGCPNPRLVKVTGQCCEEWVCDEDS |
| NOV12g | QNGESFQPNCKHQCTCIDGAVGCIPLCPQELSLPNLGCPNPRLVKVTGQCCEEWVCDEDS |
| NOV12h | QNGESFQPNCKHQCTCIDGAVGCIPLCPQELSLPNLGCPNPRLVKVTGQCCEEWVCDEDS |
| NOV12i | QNGESFQPNCKHQCTCIDGAVGCIPLCPQELSLPNLGCPNPRLVKVTGQCCEEWVCDEDS |
| NOV12j | QNGESFQPNCKHQCTCIDGAVGCIPLCPQELSLPNLGCPNPRLVKVTGQCCEEWVCDEDS |
| NOV12k | QNGESFQPNCKHQCTCIDGAVGCIPLCPQELSLPNLGCPNPRLVKVTGQCCEEWVCDEDS |
| NOV12l | QNGESFQPNCKHQCTCIDGAVGCIPLCPQELSLPNLGCPNPRLVKVTGQCCEEWVCDEDS |
| NOV12a | IKDPMEDQDGLLGKELGFDASEVELTRNNELIAVGKGSSLKRIPVFGMEPRIRYNPLQGQ |
| NOV12b | IKDPMEDQDGLLGKELGFDASEVELTRNNELIAVGKGSSLKRLPVFGMEPRILYNPLQGQ |
| NOV12c | ------------------------------------------------------------ |
| NOV12d | ------------------------------------------------------------ |
| NOV12e | ------------------------------------------------------------ |
| NOV12f | IKDPMEDQDGLLGKELGFDASEVELTRNNELIAVGKGSSLKRLPVFGMEPRILYNPLQGQ |
| NOV12g | IKDPMEDQDGLLGKELGFDASEVELTRNNELIAVGKGSSLKRLPVFGMEPRILYNPLQGQ |
| NOV12h | IKDPMEDQDGLLGKELGFDASEVELTRNNELIAVGKGSSLKRLPVFGMEPRILYNPLQGQ |
| NOV12i | IKDPMEDQDGLLGKELGFDASEVELTRNNELIAVGKGSSLKRLPVFGMEPRILYNPLQGQ |
| NOV12j | IKDPMEDQDGLLGKELGFDASEVELTRNNELIAVGKGSSLKRLPVFGMEPRILYNPLQGQ |
| NOV12k | IKDPMEDQDGLLGKELGFDASEVELTRNNELIAVGKGSSLKRLPVFGMEPRILYNPLQGQ |
| NOV12l | IKDPMEDQDGLLGKELGFDASEVELTRNNELIAVGKGSSLKRLPVFGMEPRILYNPLQGQ |
| NOV12a | KCIVQTTSWSQCSKTCGTGISTRVTNDNPECRLVKETRICEVRPCGQPVYSSLKKGKKCS |
| NOV12b | KCIVQTTSWSQCSKTCGTGISTRVTNDNPECRLVKETRICEVRPCGQPVYSSLKKGKKCS |
| NOV12c | ------------------------------------------------------------ |
| NOV12d | ------------------------------------------------------------ |
| NOV12e | ------------------------------------------------------------ |
| NOV12f | KCIVQTTSWSQCSKTCGTGISTRVTNDNPECRLVKETRICEVRPCGQPVYSSLKKGKKCS |
| NOV12g | KCIVQTTSWSQCSKTCGTGISTRVTNDNPECRLVKETRICEVRPCGQPVYSSLKKGKKCS |
| NOV12h | KCIVQTTSWSQCSKTCGTGISTRVTNDNPECRLVKETRICEVRPCGQPVYSSLKKGKKCS |
| NOV12i | KCIVQTTSWSQCSKTCGTGISTRVTNDNPECRLVKETRICEVRPCGQPVYSSLKKGKKCS |
| NOV12j | KCIVQTTSWSQCSKTCGTGISTRVTNDNPECRLVKETRICEVRPCGQPVYSSLKKGKKCS |
| NOV12k | KCIVQTTSWSQCSKTCGTGISTRVTNDNPECRLVKETRICEVRPCGQPVYSSLKKGKKCS |
| NOV12l | KCIVQTTSWSQCSKTCGTGISTRVTNDNPECRLVKETRICEVRPCGQPVYSSLKKGKKCS |
| NOV12a | KTKKSPEPVRFTYAGCLSVKKYRPKYCGSCVDGRCCTPQLTRTVKMRFRCEDGETFSKNV |
| NOV12b | KTKKSPEPVRFTYAGCLSVKKYRPKYCGSCVDGRCCTPQLTRTVKMRFRCEDGETFSKNV |
| NOV12c | ------------------------------------------------------------ |
| NOV12d | ------------------------------------------------------------ |
| NOV12e | ------------------------------------------------------------ |
| NOV12f | KTKKSPEPVRFTYAGCLSVKKYRPKYCGSCVDGRCCTPQLTRTVKMRFRCEDGETFSKNV |
| NOV12g | KTKKSPEPVRFTYAGCLSVKKYRPKYCGSCVDGRCCTPQLTRTVKMRFRCEDGETFSKNV |
| NOV12h | KTKKSPEPVRFTYAGCLSVKKYRPKYCGSCVDGRCCTPQLTRTVKMRFRCEDGETFSKNV |
| NOV12i | KTKKSPEPVRFTYAGCLSVKKYRPKYCGSCVDGRCCTPQLTRTVKMRFRCEDGETFSKNV |
| NOV12j | KTKKSPEPVRFTYAGCLSVKKYRPKYCGSCVDGRCCTPQLTRTVKMRFRCEDGETFSKNV |
| NOV12k | KTKKSPEPVRFTYAGCLSVKKYRPKYCGSCVDGRCCTPQLTRTVKMRFRCEDGETFSKNV |
| NOV12l | KTKKSPEPVRFTYAGCLSVKKYRPKYCGSCVDGRCCTPQLTRTVKMRFRCEDGETFSKNV |
| NOV12a | MMIQSCKCNYNCPHANEAAFPFYRLFNDIHKFRD-------------- |
| NOV12b | MMIQSCKCNYNCPHANEAAFPFYRLFNDIHKFRDLE------------ |
| NOV12c | ------------------------------------------------ |
| NOV12d | ------------------------------------------------ |
| NOV12e | ------------------------------------------------ |
| NOV12f | MMIQSCKCNYNCPHANEAAFPFYRLFNDIHKFRDKGGRALSRVRRPRS |
| NOV12g | MMIQSCKCNYNCPHANEAAFPFYRLFNDIHKFRDKGGRA--------- |
| NOV12h | MMIQSCKCNYNCPHANEAAFPFYRLFNDIHKFR--------------- |
| NOV12i | MMIQSCKCNYNCPHANEAAFPFYRLFNDIHKFRDKGGRA--------- |
| NOV12j | MMIQSCKCNYNCPHANEAAFPFYRLFNDIHKFRDKGGRA--------- |
| NOV12k | MMIQSCKCNYNCPHANEAAFPFYRLFNDIHKFRD-------------- |
| NOV12l | MMIQSCKCNYNCPHANEAAFPFYRLFNDIHKFRD-------------- |
| NOV12a | (SEQ ID NO: 150) |
| NOV12b | (SEQ ID NO: 152) |
| NOV12c | (SEQ ID NO: 154) |
| NOV12d | (SEQ ID NO: 156) |
| NOV12e | (SEQ ID NO: 158) |
| NOV12f | (SEQ ID NO: 160) |
| NOV12g | (SEQ ID NO: 162) |
| NOV12h | (SEQ ID NO: 164) |
| NOV12i | (SEQ ID NO: 166) |
| NOV12j | (SEQ ID NO: 168) |
| NOV12k | (SEQ ID NO: 170) |
| NOV12l | (SEQ ID NO: 172) |

Further analysis of the NOV12a protein yielded the following properties shown in Table 12C.

TABLE 12C
Protein Sequence Properties NOV12a

| SignalP | Cleavage site between residues 25 and 26 | | | |
|---|---|---|---|---|
| analysis: | | | | |
| PSORT II | PSG: a new signal peptide prediction method | | | |
| analysis: | N-region: length 7; pos. chg 2; neg. chg 0 | | | |
| | H-region: length 12; peak value 10.04 | | | |
| | PSG score: 5.64 | | | |
| | GvH: von Heijne's method for signal seq. recognition | | | |
| | GvH score (threshold: −2.1): −1.17 | | | |
| | possible cleavage site: between 21 and 22 | | | |
| | >>> Seems to have a cleavable signal peptide (1 to 21) | | | |
| | ALOM: Klein et al's method for TM region allocation | | | |
| | Init position for calculation: 22 | | | |
| | Tentative number of TMS(s) for the threshold 0.5: 0 | | | |
| | number of TMS(s) . . . fixed | | | |
| | PERIPHERAL | Likelihood = 5.73 (at 124) | | |
| | ALOM score: 5.73 (number of TMSs: 0) | | | |
| | MTOP: Prediction of membrane topology (Hartmann et al.) | | | |
| | Center position for calculation: 10 | | | |
| | Charge difference: −1.0 C(2.0)-N(3.0) | | | |
| | N >= C: N-terminal side will be inside | | | |
| | MITDISC: discrimination of mitochondrial targeting seq | | | |
| | R content: | 3 | Hyd Moment(75): | 7.35 |
| | Hyd Moment(95): | 12.47 | G content: | 0 |
| | D/E content: | 1 | S/T content: | 6 |

Score: 0.47
Gavel: prediction of cleavage sites for mitochondrial preseq
R-2 motif at 57 VRD|GC
NUCDISC: discrimination of nuclear localization signals
pat4: none
pat7: none
bipartite: none
content of basic residues: 13.9%
NLS Score: −0.47
KDEL: ER retention motif in the C-terminus: none
ER Membrane Retention Signals:
XXRR-like motif in the N-terminus: SSRI
KKXX-like motif in the C-terminus: HKFR
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: nuclear
Reliability: 94.1
COIL: Lupas's algorithm to detect coiled-coil regions
total: 0 residues
Final Results (k = 9/23):
56.5%: mitochondrial
17.4%: extracellular, including cell wall
17.4%: nuclear
8.7%: cytoplasmic
>> prediction for CG55688-01 is mit (k = 23)

A search of the NOV12a protein against the Geneseq database, a proprietary database that contains sequences published in patents and patent publication, yielded several homologous proteins shown in Table 12D.

TABLE 12D
Geneseq Results for NOV12a

| | | NOV12a | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| ABG76937 | Human protein, comprising CYR61, | 1 . . . 381 | 381/381 (100%) | 0.0 |
| | designated SEC1 - Homo sapiens, 381 | 1 . . . 381 | 381/381 (100%) | |
| | aa. [WO200255705-A2, | | | |
| | 18 JUL. 2002] | | | |
| ABB05438 | Human Cyr61 protein SEQ ID NO: 2 - | 1 . . . 381 | 379/381 (99%) | 0.0 |
| | Homo sapiens, 381 aa. | 1 . . . 381 | 380/381 (99%) | |
| | [WO200198359-A2, 27 DEC. 2001] | | | |
| AAE18107 | Human connective tissue growth | 1 . . . 381 | 379/381 (99%) | 0.0 |
| | factor-2 (CTGF-2) - Homo sapiens, | 1 . . . 381 | 380/381 (99%) | |
| | 381 aa. [WO200204480-A2, | | | |
| | 17 JAN. 2002] | | | |
| AAU79761 | Human Cyr61 protein - Homo | 1 . . . 381 | 379/381 (99%) | 0.0 |
| | sapiens, 381 aa. [WO200226193-A2, | 1 . . . 381 | 380/381 (99%) | |
| | 04 APR. 2002] | | | |
| AAB90773 | Human shear stress-response protein | 1 . . . 381 | 379/381 (99%) | 0.0 |
| | SEQ ID NO: 46 - Homo sapiens, 381 | 1 . . . 381 | 380/381 (99%) | |
| | aa. [WO200125427-A1, | | | |
| | 12 APR. 2001] | | | |

In a BLAST search of public sequence databases, the NOV12a protein was found to have homology to the proteins shown in the BLASTP data in Table 12E.

TABLE 12E
Public BLASTP Results for NOV12a

| | | NOV12a | Identities/ | |
|---|---|---|---|---|
| Protein | | Residues/ | Similarities for | |
| Accession | | Match | the Matched | Expect |
| Number | Protein/Organism/Length | Residues | Portion | Value |
| O00622 | CYR61 protein precursor | 1 . . . 381 | 379/381 (99%) | 0.0 |
| | (Cysteine-rich, angiogenic inducer, 61) | 1 . . . 381 | 380/381 (99%) | |
| | (Insulin-like growth factor-binding | | | |
| | protein 10) (GIG1 protein) - Homo | | | |
| | sapiens (Human), 381 aa. | | | |
| CAC60183 | Sequence 3 from Patent WO0155210 - | 1 . . . 381 | 377/381 (98%) | 0.0 |
| | Homo sapiens (Human), 381 aa. | 1 . . . 381 | 379/381 (98%) | |
| CAD42176 | Sequence 1 from Patent EP1217067 - | 1 . . . 373 | 358/374 (95%) | 0.0 |
| | Homo sapiens (Human), 374 aa | 1 . . . 374 | 360/374 (95%) | |
| | (fragment). | | | |
| Q9ES72 | CYR61 protein precursor | 1 . . . 381 | 348/383 (90%) | 0.0 |
| | (Cysteine-rich, angiogenic inducer, 61) | 1 . . . 379 | 358/383 (92%) | |
| | (Insulin-like growth factor-binding | | | |
| | protein 10) - Rattus norvegicus (Rat), | | | |
| | 379 aa. | | | |
| P18406 | CYR61 protein precursor | 1 . . . 381 | 348/383 (90%) | 0.0 |
| | (Cysteine-rich, angiogenic inducer, 61) | 1 . . . 379 | 358/383 (92%) | |
| | (Insulin-like growth factor-binding | | | |
| | protein 10) (3CH61) - Mus musculus | | | |
| | (Mouse), 379 aa. | | | |

PFam analysis predicts that the NOV12a protein contains the domains shown in the Table 12F.

TABLE 12F
Domain Analysis of NOV12a

| | | Identities/ | |
|---|---|---|---|
| | | Similarities | |
| Pfam | | for the | Expect |
| Domain | NOV12a Match Region | Matched Region | Value |
| IGFBP | 26 . . . 97 | 32/85 (38%) | 1.4e−25 |
| | | 60/85 (71%) | |
| vwc | 100 . . . 163 | 35/85 (41%) | 2.4e−26 |
| | | 60/85 (71%) | |
| tsp_1 | 231 . . . 272 | 16/53 (30%) | 5.6e−11 |
| | | 36/53 (68%) | |
| Cys_knot | 279 . . . 376 | 25/115 (22%) | 1.5e−29 |
| | | 88/115 (77%) | |

### Example 13

The NOV13 clone was analyzed, and the nucleotide and encoded polypeptide sequences are shown in Table 13A.

TABLE 13A
NOV13 Sequence Analysis

| | | |
|---|---|---|
| NOV13a, CG56768-01 | SEQ ID NO: 175 | 1214 bp |
| DNA Sequence | ORF Start: ATG at 60 | ORF Stop: TAG at 1155 |

CTCCTTTCTTCCCTCTCCAGAAGTCCATTGGAATATTAAGCCCAGGAGTTGCTTTGGGGATGCCTGGAAGTGC
AATGTCTTCCAAGTTCTTCCTAGTGGCTTTGGCCATATTTTTCTCCTTCGCCCAGGTTGTAATTGAAGCCAAT
TCTTGGTGGTCGCTAGGTATGAATAACCCTGTTCAGATGTCAGAAGTATATATTATAGGAGCACAGCCTCTCT
GCAGCCAACTGGCAGGACTTTCTCAAGGACAGAAGAAACTGTGCCACTTGTATCAGGACCACATGCAGTACAT
CGGAGAAGGCGCGAAGACAGGCATCAAAGAATGCCAGTATCAATTCCGACATCGAAGGTGGAACTGCAGCACT
GTGGATAACACCTCTGTTTTTGGCAGGGTGATGCAGATAGGCAGCCGCGAGACGGCCTTCACATACGCGGTGA
GCGCAGCAGGGGTGGTGAACGCCATGAGCCGGGCGTGCCGCGAGGGCGAGCTGTCCACCTGCGGCTGCAGCCG
CGCCGCGCGCCCCAAGGACCTGCCGCGGGACTGGCTCTGGGGCGGCTGCGGCGACAACATCGACTATGGCTAC
CGCTTTGCCAAGGAGTTCGTGGACGCCCGCGAGCGGGAGCGCATCCACGCCAAGGGCTCCTACGAGAGTGCTC
GCATCCTCATGAACCTGCACAACAACGAGGCCGGCCGCAGGACGGTGTACAACCTGGCTGATGTGGCCTGCAA
GTGCCATGGGGTGTCCGGCTCATGTAGCCTGAAGACATGCTGGCTGCAGCTGGCAGACTTCCGCAAGGTGGGT
GATGCCCTGAAGGAGAAGTACGACAGCGCGGCGGCCATGCGGCTCAACAGCCGGGGCAAGTTGGTACAGGTCA
ACAGCCGCTTCAACTCGCCCACCACACAAGACCTGGTCTACATCGACCCCAGCCCTGACTACTGCGTGCGCAA
TGAGAGCACCGGCTCGCTGGGCACGCAGGGCCGCCTGTGCAACAAGACGTCGGAGGGCATGGATGGCTGCGAG
CTCATGTGCTGCGGCCGTGGCTACGACCAGTTCAAGACCGTGCAGACGGAGCGCTGCCACTGCAAGTTCCACT
GGTGCTGCTACGTCAAGTGCAAGAAGTGCACGGAGATCGTGGACCAGTTTGTGTGCAAGTAGTGGGTGCCACC
CAGCACTCAGCCCCGCCCCCAGGACCCGCTTATTTATAGAAAGTAC

| | | | |
|---|---|---|---|
| NOV13a, CG56768-01 | | | |
| Protein Sequence | SEQ ID NO: 176 | 365 aa | MW at 40886.3kD |

MAGSAMSSKFFLVALAIFFSFAQVVIEANSWWSLGMNNPVQMSEVYIIGAQPLCSQLAGLSQGQKKLCHLYQD
HMQYIGEGAKTGIKECQYQFRHRRWNCSTVDNTSVFGRVMQIGSRETAFTYAVSAAGVVNAMSRACREGELST
CGCSRAARPKDLPRDWLWGGCGDNIDYGYRFAKEFVDARERERIHAKGSYESARILMNLHNNEAGRRTVYNLA
DVACKCHGVSGSCSLKTCWLQLADFRKVGDALKEKYDSAAAMRLNSRGKLVQVNSRFNSPTTQDLVYIDPSPD
YCVRNESTGSLGTQGRLCNKTSEGMDGCELMCCGRGYDQFKTVQTERCHCKFHWCCYVKCKKCTEIVDQFVCK

| | | |
|---|---|---|
| NOV13b, CG56768-02 | SEQ ID NO: 177 | 1026 bp |
| DNA Sequence | ORF Start: at 7 | ORF Stop: at 1021 |

GGATCCGCCAATTCTTGGTGGTCGCTAGGTATGAATAACCCTGTTCAGATGTCAGAAGTATATATTATAGGAG
CACAGCCTCTCTGCAGCCAACTGGCAGGACTTTCTCAAGGACAGAAGAAACTGTGCCACTTGTATCAGGACCA
CATGCAGTACATCGGAGAAGGCGCGAAGACAGGCATCAAAGAATGCCAGTATCAATTCCGACATCGAAGGTGG
AACTGCAGCACTGTGGATAACACCTCTGTTTTTGGCAGGGTGATGCAGATAGGCAGCCGCGAGACGGCCTTCA
CATACGCGGTGAGCGCAGCAGGGGTGGTGAACGCCATGAGCCGGGCGTGCCGCGAGGGCGAGCTGTCCACCTG
CGGCTGCAGCCGCGCCGCGCGCCCCAAGGACCTGCCGCGGGACTGGCTCTCCCGCGGCTGCGGCGACAACATC
GACTATGGCTACCGCTTTGCCAAGGAGTTCGTGGACGCCCGCGAGCGGCAGCGCATCCACGCCAAGGGCTCCT
ACGAGAGTGCTCGCATCCTCATGAACCTGCACAACAACGAGGCCGGCCGCAGGACGGTGTACAACCTGGCTGA
TGTGGCCTGCAAGTGCCATGGGGTGTCCGGCTCATGTAGCCTGAAGACATGCTGGCTGCAGCTGGCAGACTTC
CGCAAGGTGGGTGATGCCCTGAAGGAGAAGTACGACAGCGCGGCGGCCATGCGGCTCAACAGCCGGGGCAAGT
TGGTACAGGTCAACAGCCGCTTCAACTCGCCCACCACACAAGACCTGGTCTACATCGACCCCAGCCCTGACTA
CTGCGTGCGCAATGAGAGCACCGGCTCGCTGGGCACGCAGGGCCGCCTGTGCAACAAGACGTCGGAGGGCATG
GATGGCTGCGAGCTCATGTGCTGCGGCCGTGGCTACGACCAGTTCAAGACCGTGCAGACGGAGCGCTGCCACT
GCAAGTTCCACTGGTGCTGCTACGTCAAGTGCAAGAAGTGCACGGAGATCGTGGACCAGTTTGTGTGCAAGCT
CGAG

| | | | |
|---|---|---|---|
| NOV13b, CG56768-02 | | | |
| Protein Sequence | SEQ ID NO: 178 | 338 aa | MW at 37991.8kD |

ANSWWSLGMNNPVQMSEVYIIGAQPLCSQLAGLSQGQKKLCHLYQDHMQYIGEGAKTGIKECQYQFRHRRWNC
STVDNTSVFGRVMQIGSRETAFTYAVSAAGVVNAMSRACREGELSTCGCSRAARPKDLPRDWLWGGCGDNIDY
GYRFAKEFVDARERERIHAKGSYESARILMNLHNNEAGRRTVYNLADVACKCHGVSGSCSLKTCWLQLADFRK
VGDALKEKYDSAAAMRLNSRGKLVQVNSRFNSPTTQDLVYIDPSPDYCVRNESTGSLGTQGRLCNKTSEGMDG
CELMCCGRGYDQFKTVQTERCHCKFHWCCYVKCKKCTEIVDQFVCK

| | | |
|---|---|---|
| NOV13c, CG56768-03 | SEQ ID NO: 179 | 1215 bp |
| DNA Sequence | ORF Start: at 16 | ORF Stop: TAG at 1156 |

GCTCCTTTCTTCCCTCTCCAGAAGTCCATTGGAATATTAAGCCCAGGAGTTGCTTTGGGGATGGCTGGAAGTG
CAATGTCTTCCAAGTTCTTCCTAGTGGCTTTGGCCATATTTTTCTCCTTCGCCCAGGTTGTAATTGAAGCCAA
TTCTTGGTGGTCGCTAGGTATGAATAACCCTGTTCAGATGTCAGAAGTATATATTATAGGAGCACAGCCTCTC
TGCAGCCAACTGGCAGGACTTTCTCAAGGACAGAACAAACTGTGCCACTTGTATCAGGACCACATGCAGTACA
TCGGAGAAGGCGCGAAGACAGGCATCAAAGAATGCCAGTATCAATTCCGACATCGAAGGTGGAACTGCAGCAC
TGTGGATAACACCTCTGTTTTTGGCAGGGTGATGCAGATAGGTAGCCGCGAGACGGCCTTCACATACGCGGTG
AGCGCAGCAGGGGTGGTGAACGCCATGAGCCGGGCGTGCCGCGAGGGCGAGCTGTCCACCTGCGGCTGCAGCC
GCGCCGCGCGCCCCAAGGACCTGCCGCGGGACTGGCTCTGGGGCGGCTCCGGCGCCACCAACAAAAAAGGCTA
CCGCTCCGCCAAGGAGATCGTGCACGCCCGCGAACGAGGACGCATCCACGCCAAGGGCTCCTACGAGAGTGCT
CGCATCCTCATGAACCTGCACAACAACGAGGCCGGCCGCAGGACGGTGTACAACCTGGCTGATGTGCCCTGCA
AGTGCCATGGGGTGTCCGGCTCATGTAGCCTGAAGACATGCTGGCTGCAGCTGGCAGACTTCCGCAAGGTGGG
TGATGCCCTGAAGGAGAAGTACGACAGCGCGGCGGCCATGCGGCTCAACAGCCGGGGCAAGTTGGTACAGGTC
AACAGCCGCTTCAACTCGCCCACCACACAAGACCTGGTCTACATCGACCCCAGCCCTGACTACTGCGTGCGCA
ATGAGAGCACCGGCTCGCTGGGCACGCAGGGCCGCCTGTGCAACAAGACGTCGGAGGGCATGGATGGCTGCGA
GCTCATGTGCTGCGGCCGTGGCTACGACCAGTTCAAGACCGTGCAGACGGAGCGCTGCCACTGCAAGTTCCAC
TGGTGCTGCTACGTCAAGTGCAAGAAGTGCACGGAGATCGTGGACCAGTTTGTGTGCAAGTAGTGGGTGCCAC
CCAGCACTCAGCCCCGCTCCCAGGACCCGCTTATTTATAGAAAGTAC

| | | | |
|---|---|---|---|
| NOV13c, CG56768-03 | | | |
| Protein Sequence | SEQ ID NO: 180 | 380 aa | MW at 42082.8kD |

LQKSIGILSPGVALGMAGSAMSSKFFLVALAIFFSFAQVVIEANSWWSLGMNNPVQMSEVYIIGAQPLCSQLA
GLSQGQKKLCHLYQDHMQYIGEGAKTGIKECQYQFRHRRWNCSTVDNTSVFGRVMQICSRETAFTYAVSAAGV
VNAMSRACREGELSTCGCSRAARPKDLPRDWLWGGSGATNKKGYRSAKEIVHARERGRIHAKGSYESARILMN
LHNNEAGRRTVYNLADVACKCHGVSGSCSLKTCWLQLADFRKVCDALKEKYDSAAAMRLNSRGKLVQVNSRFN
SPTTQDLVYIDPSPDYCVRNESTGSLGTQGRLCNKTSEGMDGCELMCCGRGYDQFKTVQTERCHCKFHWCCYV
KCKKCTEIVDQFVCK

A ClustalW comparison of the above protein sequences yields the following sequence alignment shown in Table 13B.

TABLE 13B
Comparison of the NOV13 protein sequences.

| | |
|---|---|
| NOV13a | ---------------MAGSAMSSKFFLVALAIFFSFAQVVIEANSWWSLGMNNPVQMSEV |
| NOV13b | ------------------------------------------ANSWWSLGMNNPVQMSEV |
| NOV13c | LQKSIGILSPGVALGMAGSAMSSKFFLVALAIFFSFAQVVIEANSWWSLGMNNPVQMSEV |
| NOV13a | YIIGAQPLCSQLAGLSQGQKKLCHLYQDHMQYIGEGAKTGIKECQYQFRHRRWNCSTVDN |
| NOV13b | YIIGAQPLCSQLAGLSQGQKKLCHLYQDHMQYIGEGAKTGIKECQYQFRHRRWNCSTVDN |
| NOV13c | YIIGAQPLCSQLAGLSQGQKKLCHLYQDHMQYIGEGAKTGIKECQYQFRHRRWNCSTVDN |
| NOV13a | TSVFGRVMQIGSRETAFTYAVSAAGVVNAMSRACREGELSTCGCSRAARPKDLPRDWLWG |
| NOV13b | TSVFGRVMQIGSRETAFTYAVSAAGVVNAMSRACREGELSTCGCSRAARPKDLPRDWLWG |
| NOV13c | TSVFGRVMQIGSRETAFTYAVSAAGVVNAMSRACREGELSTCGCSRAARPKDLPRDWLWG |
| NOV13a | GCGDNIDYGYRFAKEFVDARERERIHAKGSYESARILMNLHNNEAGRRTVYNLADVACKC |
| NOV13b | GCGDNIDYGYRFAKEFVDARERERIHAKGSYESARILMNLHNNEAGRRTVYNLADVACKC |
| NOV13c | GSGATNKKGYRSAKEIVHARERGRIHAKGSYESARILMNLHNNEAGRRTVYNLADVACKC |
| NOV13a | HGVSGSCSLKTCWLQLADFRKVGDALKEKYDSAAAMRLNSRGKLVQVNSRFNSPTTQDLV |
| NOV13b | HGVSGSCSLKTCWLQLADFRKVGDALKEKYDSAAAMRLNSRGKLVQVNSRFNSPTTQDLV |
| NOV13c | HGVSGSCSLKTCWLQLADFRKVGDALKEKYDSAAAMRLNSRGKLVQVNSRFNSPTTQDLV |
| NOV13a | YIDPSPDYCVRNESTGSLGTQGRLCNKTSEGMDGCELMCCGRGYDQFKTVQTERCHCKFH |
| NOV13b | YIDPSPDYCVRNESTGSLGTQGRLCNKTSEGMDGCELMCCGRGYDQFKTVQTERCHCKFH |
| NOV13c | YIDPSPDYCVRNESTGSLGTQGRLCNKTSEGMDGCELMCCGRGYDQFKTVQTERCHCKFH |
| NOV13a | WCCYVKCKKCTEIVDQFVCK |
| NOV13b | WCCYVKCKKCTEIVDQFVCK |
| NOV13c | WCCYVKCKKCTEIVDQFVCK |
| NOV13a | (SEQ ID NO: 176) |
| NOV13b | (SEQ ID NO: 178) |
| NOV13c | (SEQ ID NO: 180) |

Further analysis of the NOV13a protein yielded the following properties shown in Table 13C.

TABLE 13C
Protein Sequence Properties NOV13a

| SignalP | Cleavage site between residues 28 and 29 | | | |
|---|---|---|---|---|
| analysis: | | | | |
| PSORT II | PSG: a new signal peptide prediction method | | | |
| analysis: | N-region: length 9; pos.chg 1; neg.chg 0 | | | |
| | H-region: length 17; peak value 11.46 | | | |
| | PSG score: 7.06 | | | |
| | GvH: von Heijne's method for signal seq. recognition | | | |
| | GvH score (threshold: −2.1): 2.33 | | | |
| | possible cleavage site: between 22 and 23 | | | |
| | >>> Seems to have a cleavable signal peptide (1 to 22) | | | |
| | ALOM: Klein et al's method for TM region allocation | | | |
| | Init position for calculation: 23 | | | |
| | Tentative number of TMS(s) for the threshold 0.5: 0 | | | |
| | number of TMS(s) . . . fixed | | | |
| | PERIPHERAL | Likelihood = 4.51 (at 45) | | |
| | ALOM score: 4.51 (number of TMSs: 0) | | | |
| | MTOP: Prediction of membrane topology (Hartmann et al.) | | | |
| | Center position for calculation: 11 | | | |
| | Charge difference: −3.0 C(−1.0)-N(2.0) | | | |
| | N >= C: N-terminal side will be inside | | | |
| | MITDISC: discrimination of mitochondrial targeting seq | | | |
| | R content: | 0 | Hyd Moment(75): | 3.30 |
| | Hyd Moment(95): | 2.27 | G content: | 1 |
| | D/E content: | 1 | S/T content: | 4 |

Score: −5.10
Gavel: prediction of cleavage sites for mitochondrial preseq
cleavage site motif not found
NUCDISC: discrimination of nuclear localization signals
pat4: RHRR (3) at 94
pat7: none
bipartite: none
content of basic residues: 12.6%
NLS Score: −0.29
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 55.5
COIL: Lupas's algorithm to detect coiled-coil regions
total: 0 residues
Final Results (k = 9/23):
22.2%: extracellular, including cell wall
22.2%: vacuolar
22.2%: mitochondrial
22.2%: endoplasmic reticulum
11.1%: Golgi
>> prediction for CG56768-01 is exc (k = 9)

A search of the NOV13a protein against the Geneseq database, a proprietary database that contains sequences published in patents and patent publication, yielded several homologous proteins shown in Table 13D.

TABLE 13D
Geneseq Results for NOV13a

| | | NOV13a | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| AAE34041 | WNT-4 protein - Unidentified, 365 aa. | 1 . . . 365 | 365/365 (100%) | 0.0 |
| | [WO200290992-A2, 14 NOV. 2002] | 1 . . . 365 | 365/365 (100%) | |
| ABP58342 | Human cell growth, differentiation | 1 . . . 365 | 365/365 (100%) | 0.0 |
| | and death protein CGDD-13 - Homo | 1 . . . 365 | 365/365 (100%) | |
| | sapiens, 365 aa. [WO200297032-A2, | | | |
| | 05 DEC. 2002] | | | |
| ABU56526 | Lung cancer-associated polypeptide | 1 . . . 365 | 365/365 (100%) | 0.0 |
| | #119 - Unidentified, 365 aa. | 1 . . . 365 | 365/365 (100%) | |
| | [WO200286443-A2, 31 OCT. 2002] | | | |
| ABU55887 | Human WNT-5A protein - Homo | 1 . . . 365 | 365/365 (100%) | 0.0 |
| | sapiens, 365 aa. [WO200277204-A2, | 1 . . . 365 | 365/365 (100%) | |
| | 03 OCT. 2002] | | | |
| ABU04874 | Human expressed protein tag (EPT) | 1 . . . 365 | 365/365 (100%) | 0.0 |
| | #1540 - Homo sapiens, 365 aa. | 1 . . . 365 | 365/365 (100%) | |
| | [WO200278524-A2, 10 OCT. 2002] | | | |

In a BLAST search of public sequence databases, the NOV13a protein was found to have homology to the proteins shown in the BLASTP data in Table 13E.

TABLE 13E
Public BLASTP Results for NOV13a

| | | NOV13a | | |
|---|---|---|---|---|
| Protein | | Residues/ | Identities/ | |
| Accession | | Match | Similarities for the | Expect |
| Number | Protein/Organism/Length | Residues | Matched Portion | Value |
| P41221 | Wnt-5a protein precursor - Homo | 1 . . . 365 | 365/365 (100%) | 0.0 |
| | sapiens (Human), 365 aa. | 1 . . . 365 | 365/365 (100%) | |
| Q8VCV6 | Wnt-5a protein - Mus musculus | 2 . . . 365 | 361/364 (99%) | 0.0 |
| | (Mouse), 380 aa. | 17 . . . 380 | 362/364 (99%) | |
| Q8BMF9 | WNT-5A protein precursor - Mus | 2 . . . 365 | 360/364 (98%) | 0.0 |
| | musculus (Mouse), 380 aa. | 17 . . . 380 | 361/364 (98%) | |
| Q8BM17 | WNT-5A protein precursor - Mus | 6 . . . 365 | 358/360 (99%) | 0.0 |
| | musculus (Mouse), 360 aa. | 1 . . . 360 | 359/360 (99%) | |
| Q9QXQ7 | Wnt-5a protein precursor - Rattus | 2 . . . 365 | 358/364 (98%) | 0.0 |
| | norvegicus (Rat), 379 aa. | 17 . . . 379 | 360/364 (98%) | |

PFam analysis predicts that the NOV13a protein contains the domains shown in the Table 13F.

TABLE 13F
Domain Analysis of NOV13a

| | | Identities/ | |
|---|---|---|---|
| Pfam | | Similarities | Expect |
| Domain | NOV13a Match Region | for the Matched Region | Value |
| wnt | 53 . . . 365 | 189/352 (54%) | 2e−212 |
| | | 295/352 (84%) | |

### Example 14

The NOV14 clone was analyzed, and the nucleotide and encoded polypeptide sequences are shown in Table 14A.

TABLE 14A
NOV14 Sequence Analysis

| | | |
|---|---|---|
| NOV14a, CG57054-03 | SEQ ID NO: 181 | 1215 bp |
| DNA Sequence | ORF Start: ATG at 55 | ORF Stop: TGA at 1165 |

CCAAACCACTGGAGGTCCTGATCGATCTGCCCACCGGAGCCTCCGGGCTTCGACATGCTGGAGGAGCCCCGGC
CGCGGCCTCCGCCCTCGGGCCTCGCGGGTCTCCTGTTCCTGGCGTTGTGCAGTCGGGCTCTAAGCAATGAGAT
TCTGGGCCTGAAGTTGCCTGGCGAGCCGCCGCTGACGGCCAACACCGTGTGCTTGGCGCTGTCCGGCCTGAGC
AAGCGGCAGCTAGGCCTGTGCCTGCGCAACCCCGACGTGACGGCGTCCGCGCTTCAGGGTCTGCACATCGCGG
TCCACGAGTGTCAGCACCAGCTGCGCGACCAGCGCTGGAACTGCTCCGCGCTTGAGGGCGGCGGCCGCCTGCC
GCACCACAGCGCCATCCTCAAGCGGGCCTGGTGTAGGGGAAGGCTTGGACACCCAAATGGTTTCCGAGAAAGT
GCTTTTTCCTTCTCCATGCTGGCTGCTGGGGTCATGCACGCAGTAGCCACGGCCTGCAGCCTGGGCAAGCTGG
TGAGCTGTGGCTGTGGCTGGAAGGGCAGTGGTGAGCAGGATCGGCTGAGGGCCAAACTGCTGCAGCTGCAGGC
ACTGTCCCGAGGCAAGAGTTTCCCCCACTCTCTGCCCAGCCCTGGCCCTGGCTCAAGCCCCAGCCCTCGCCCC
CAGGACACATGGGAATGGGGTGGCTGTAACCATGACATGGACTTTGGAGAGAAGTTCTCTCGGGATTTCTTGG
ATTCCAGGGAAGCTCCCCGGGACATCCAGGCACGAATGCGAATCCACAACAACAGGGTGGGGCGCCAGGTGGT
AACTGAAAACCTGAAGCGGAAATGCAAGTGTCATGGCACATCTGGCAGCTGCCAGTTCAAGACAAATTCTGGA
GCCTTCCAGCCCCGTCTGCGTCCCCGTCGCCTCTCAGGAGAGCTGGTCTACTTTGAGAAGTCTCCTGACTTCT
GTGAGCGAGACCCCACTATGGGCTCCCCAGGGACAAGGGGCCGGGCCTGCAACAAGACCAGCCGCCTGTTGGA
TGGCTGTGGCAGCCTGTGCTGTGGCCGTGGGCACAACGTGCTCCGGCAGACACGAGTTGAGCGCTGCCATTGC
CGCTTCCACTGGTGCTGCTATGTCCTGTGTGATGAGTGCAAGGTTACAGAGTGGGTGAATGTGTGTAAGTGAG
GGTCAGCCTTACCTTGGGGCTGGGGAAGAGGACTGTGTGAGAGGGGT

| | | | |
|---|---|---|---|
| NOV14a, CG57054-03 | | | |
| Protein Sequence | SEQ ID NO: 182 | 370 aa | MW at 40782.4kD |

MLEEPRPRPPPSGLAGLLFLALCSRALSNEILGLKLPGEPPLTANTVCLALSGLSKRQLGLCLRNPDVTASAL
QGLHIAVHECQHQLRDQRWNCSALEGGGRLPHHSAILKRAWCRGRLGHPNGFRESAFSFSMLAAGVMHAVATA
CSLGKLVSCGCGWKGSGEQDRLRAKLLQLQALSRGKSFPHSLPSPGPGSSPSPGPQDTWEWGGCNHDMDFGEK
FSRDFLDSREAPRDIQARMRIHNNRVGRQVVTENLKRKCKCHGTSGSCQFKTNSGAFQPRLRPRRLSGELVYF
EKSPDFCERDPTMGSPGTRGRACNKTSRLLDGCGSLCCGRGHNVLRQTRVERCHCRFHWCCYVLCDECKVTEW

| | | |
|---|---|---|
| NOV14b, CG57054-01 | SEQ ID NO: 183 | 1091 bp |
| DNA Sequence | ORF Start: at 3 | ORF Stop: TGA at 1038 |

CCAACACCGTGTGCTTGACGCTGTCCGGCCTGAGCAAGCGGCAGCTAGGCCTGTGCCTGCGCAACCCCGACGT
GACCGCGTCCGCGCTTCAGGGTCTGCACATCGCGGTCCACGAGTGTCAGCACCAGCTGCGCGACCAGCGCTGG
AACTGCTCCGCGCTTGAGGGCGGCGGCCGCCTGCCGCACCACAGCGCCATCCTCAAGCGCGGTTTCCGAGAAA
GTGCTTTTTCCTTCTCCATGCTGGCTGCTGGGGTCATGCACGCAGTAGCCACGGCCTGCGGCCTGGGCAAGCT
GGTGAGCTGTGGCTGTGGCTGGAAGGGCAGTGGTGAGCAGGATCGGCTGAGGCCCAAACTGCTGCAGCTGCAG
GCACTGTCCCGAGGCAAGAGTTTCCCCCACTCTCTGCCCAGCCCTGGCCCTGGCTCAAGCCCCAGCCCTGGCC
CCCAGGACACATGGCAATGGGGTGGCTGTAACCATCACATGGACTTTGGAGAGAAGTTCTCTCGGGATTTCTT
GGATTCCAGGGAAGCTCCCCGGGACATCCAGGCACGAATGCGAATCCACAACAACAGGGTGGGGCGCCAGGTG
GTAACTGAAAACCTGAAGCGGAAATGCAAGTGTCATGGCACATCAGGCAGCTGCCAGTTCAAGACATGCTCGA
GGGCGGCCCCAGAGTTCCGGGCAGTGGGGGCGGCGTTGAGGGAGCGGCTGGGCCGGGCCATCTTCATTGATAC
CCACAACCGCAATTCTGGAGCCTTCCAGCCCCGTCTGCGTCCCCGTCGCCTCTCAGGAGAGCTGGTCTACTTT
GAGAAGTCTCCTGACTTCTGTGAGCGAGACCCCACTATGGGCTCCCCAGGGACAAGGGGCCGGGCCTGCAACA
AGACCAGCCGCCTGTTGGATGGCTCTGGCAGCCTGTGCTGTGGCCGTGGGCATAACGTGCTCCGGCAGACACG
AGTTGAGCGCTGCCATTGCCGCTTCCACTGGTGCTGCTATCTGCTGTGTGATGAGTGCAAGGTTACAGAGTGG
GTGAATGTGTGTAAGTGAGGGTCAGCCTTAGCCTTGGGGGCTGGGGAAGAGGACTGTGTGAGAGGGGCG

| | | | |
|---|---|---|---|
| NOV14b, CG57054-01 | | | |
| Protein Sequence | SEQ ID NO: 184 | 345 aa | MW at 38351.4kD |

NTVCLTLSGLSKRQLGLCLRNPDVTASALQGLHIAVHECQHQLRDQRWNCSALEGGGRLPHHSAILKRGFRES
AFSFSMLAAGVMHAVATACGLGKLVSCGCGWKGSGEQDRLRAKLLQLQALSRGKSFPHSLPSPGPGSSPSPGP
QDTWEWGGCNHDMDFGEKFSRDFLDSREAPRDIQARMRIHNNRVGRQVVTENLKRKCKCHGTSGSCQFKTCWR
AAPEFRAVGAALRERLGRAIFIDTHNRNSGAFQPRLRPRRLSGELVYFEKSPDFCERDPTMGSPGTRGRACNK
TSRLLDGCGSLCCGRGHNVLRQTRVERCHCRFHWCCYVLCDECKVTEWVNVCK

| | | |
|---|---|---|
| NOV14c, CG57054-02 | SEQ ID NO: 185 | 1317 bp |
| DNA Sequence | ORF Start: ATG at 55 | ORF Stop: TGA at 1222 |

CCAAACCACTGGAGGTCCTGATCGATCTGCCCACCGGAGCCTCCGGGCTTCGACATGCTGGAGGAGCCCCGGC
CGCGGCCTCCGCCCTCGGGCCTCGCGGGTCTCCTGTTCCTGGCGTTGTGCAGTCGGGCTCTAAGCAATGAGAT
TCTGGGCCTGAAGTTGCCTGGCGAGCCGCCGCTGACGGCCAACACCGTGTGCTTGACGCTGTCCGGCCTGAGC
AAGCGGCAGCTAGGCCTGTGCCTGCGCAACCCCGACGTGACGGCGTCCGCGCTTCAGGGTCTGCACATCGCGG
TCCACGAGTGTCAGCACCAGCTGCGCGACCAGCGCTGGAACTGCTCCGCGCTTGAGGGCGGCGGCCGCCTGCC
GCACCACAGCGCCATCCTCAAGCGCGGTTTCCGAGAAAGTGCTTTTTCCTTCTCCATGCTGGCTGCTGGGGTC
ATGCACGCAGTAGCCACGGCCTGCAGCCTGGGCAAGCTGGTCAGCTGTGGCTGTGGCTGCAAGOGCAGTGGTG
AGCAGGATCCGCTGAGGGCCAAACTGCTGCAGCTGCAGGCACTGTCCCCAGGCAAGAGTTTCCCCCACTCTCT
GCCCAGCCCTGGCCCTGGCTCAAGCCCCAGCCCTGGCCCCCAGGACACATGGGAATGGGGTGGCTGTAACCAT
GACATGGACTTTGGAGAGAAGTTCTCTCGGGATTTCTTGGATTCCAGGGAAGCTCCCCGGGACATCCAGGCAC
GAATGCGAATCCACAACAACAGGGTGGGGCGCCAGGTGGTAACTGAAAACCTGAAGCGGAAATGCAAGTGTCA
TGGCACATCACGCAGCTGCCAGTTCAAGACATGCTGGAGGGCGGCCCCAGAGTTCCGGGCAGTGGGGGCGGCG
TTGAGGGAGCGGCTGGGCCGGGCCATCTTCATTGATACCCACAACCGCAATTCTGGAGCCTTCCAGCCCCGTC
TGCGTCCCCGTCGCCTCTCAGGAGAGCTGGTCTACTTTGAGAAGTCTCCTGACTTCTGTGAGCGAGACCCCAC
TATGGGCTCCCCAGGGACAAGGGGCCGGGCCTGCAACAAGACCAGCCGCCTGTTGGATCGCTGTGGCAGCCTG
TGCTGTGCCCGTGGGCACAACGTGCTCCGGCAGACACGAGTTGAGCGCTGCCATTGCCGCTTCCACTGGTGCT
GCTATGTGCTGTGTGATGAGTGCAAGGTTACAGAGTGGGTGAATGTGTGTAAGTGAGGGTCAGCCTTACCTTG
GGGGCTGGGGAAGAGGACTCTGTGAGAGGGGCGCCTTTTCAGCCCTTTGCTCTGATTTCCTTCCAAGGTCACT
CTT

| | | | |
|---|---|---|---|
| NOV14c, CG57054-02 | | | |
| Protein Sequence | SEQ ID NO: 186 | 389 aa | MW at 42999.9kD |

MLEEPRPRPPPSGLAGLLFLALCSRALSNEILGLKLPGEPPLTANTVCLTLSGLSKRQLGLCLRNPDVTASAL
QGLHIAVHECQHQLRDQRWNCSALEGGGRLPHHSAILKRGFRESAFSFSMLAAGVMHAVATACSLGKLVSCGC
GWKGSGEQDRLRAKLLQLQALSRGKSFPHSLPSPGPGSSPSPGPQDTWEWGGCNHDMDFCEKFSRDFLDSREA
PRDIQARMRIHNNRVGRQVVTENLKRKCKCHGTSGSCQFKTCWRAAPEFRAVGAALRERLGRAIFIDTHNRNS
GAFQPRLRPRRLSGELVYFEKSPDFCERDPTMGSPGTRGRACNKTSRLLDGCGSLCCGRGHNVLRQTRVERCH
CRFHWCCYVLCDECKVTEWVNVCK

A ClustalW comparison of the above protein sequences yields the following sequence alignment shown in Table 14B.

TABLE 14B
Comparison of the NOV14 protein sequences.

| | |
|---|---|
| NOV14a | MLEEPRPRPPPSGLAGLLFLALCSRALSNEILGLKLPGEPPLTANTVCLALSGLSKRQLG |
| NOV14b | --------------------------------------------NTVCLTLSGLSKRQLG |
| NOV14c | MLEEPRPRPPPSGLAGLLFLALCSRALSNEILGLKLPGEPPLTANTVCLTLSGLSKRQLG |
| NOV14a | LCLRNPDVTASALQGLHIAVHECQHQLRDQRWNCSALEGGGRLPHHSAILKRAWCRGRLG |
| NOV14b | LCLRNPDVTASALQCLHIAVHECQHQLRDQRWNCSALEGGGRLPHHSAILKR----G--- |
| NOV14c | LCLRNPDVTASALQGLHIAVHECQHQLRDQRWNCSALEGGGRLPHHSAILKR----G--- |
| NOV14a | HPNGFRESAFSFSMLAAGVMHAVATACSLGKLVSCGCGWKGSGEQDRLRAKLLQLQALSR |
| NOV14b | ----FRESAFSFSMLAAGVMHAVATACGLGKLVSCGCGWKGSGEQDRLRAKLLQLQALSR |
| NOV14c | ----FRESAFSFSMLAAGVMHAVATACSLGKLVSCGCGWKGSGEQDRLRAKLLQLQALSR |
| NOV14a | GKSFPHSLPSPGPGSSPSPGPQDTWEWGGCNHDMDFGEKFSRDFLDSREAPRDIQARMRI |
| NOV14b | GKSFPHSLPSPGPGSSPSPGPQDTWEWGGCNHDMDFGEKFSRDFLDSREAPRDIQARMRI |
| NOV14c | GKSFPHSLPSPGPGSSPSPGPQDTWEWGGCNHDMDFGEKFSRDFLDSREAPRDIQARMRI |
| NOV14a | HNNRVGRQVVTENLKRKCKCHGTSGSCQFKT----------------------------- |
| NOV14b | HNNRVGRQVVTENLKRKCKCHGTSGSCQFKTCWRAAPEFRAVGAALRERLGRAIFIDTHN |
| NOV14c | HNNRVGRQVVTENLKRKCKCHGTSGSCQFKTCWRAAPEFRAVGAALRERLGRAIFIDTHN |
| NOV14a | -NSGAFQPRLRPRRLSGELVYFEKSPDFCERDPTMGSPGTRGRACNKTSRLLDGCGSLCC |
| NOV14b | RNSGAFQPRLRPRRLSGELVYFEKSPDFCERDPTMGSPGTRGRACNKTSRLLDGCGSLCC |
| NOV14c | RNSGAFQPRLRPRRLSGELVYFEKSPDFCERDPTMGSPGTRGRACNKTSRLLDGCGSLCC |
| NOV14a | GRGHNVLRQTRVERCHCRFHWCCYVLCDECKVTEWVNVCK |
| NOV14b | GRGHNVLRQTRVERCHCRFHWCCYVLCDECKVTEWVNVCK |
| NOV14c | GRGHNVLRQTRVERCHCRFHWCCYVLCDECKVTEWVNVCK |
| NOV14a | (SEQ ID NO: 182) |
| NOV14b | (SEQ ID NO: 184) |
| NOV14c | (SEQ ID NO: 186) |

Further analysis of the NOV14a protein yielded the following properties shown in Table 14C.

TABLE 14C
Protein Sequence Properties NOV14a

| SignalP | | | | |
|---|---|---|---|---|
| analysis: | Cleavage site between residues 29 and 30 | | | |
| PSORT II | PSG: a new signal peptide prediction method | | | |
| analysis: | N-region: length 8; pos. chg 2; neg. chg 2 | | | |
| | H-region: length 16; peak value 10.20 | | | |
| | PSG score: 5.80 | | | |
| | GvH: von Heijne's method for signal seq. recognition | | | |
| | GvH score (threshold: −2.1): 2.10 | | | |
| | possible cleavage site: between 28 and 29 | | | |
| | >>> Seems to have a cleavable signal peptide (1 to 28) | | | |
| | ALOM: Klein et al's method for TM region allocation | | | |
| | Init position for calculation: 29 | | | |
| | Tentative number of TMS(s) for the threshold 0.5: 0 | | | |
| | number of TMS(s) . . . fixed | | | |
| | PERIPHERAL | Likelihood = 0.58 (at 134) | | |
| | ALOM score: 0.58 (number of TMSs: 0) | | | |
| | MTOP: Prediction of membrane topology (Hartmann et al.) | | | |
| | Center position for calculation: 14 | | | |
| | Charge difference: −1.0 C(0.0)-N(1.0) | | | |
| | N >= C: N-terminal side will be inside | | | |
| | MITDISC: discrimination of mitochondrial targeting seq | | | |
| | R content: | 0 | Hyd Moment(75): | 9.19 |
| | Hyd Moment(95): | 7.93 | G content: | 0 |
| | D/E content: | 2 | S/T content: | 0 |

Score: −6.41
Gavel: prediction of cleavage sites for mitochondrial preseq
cleavage site motif not found
NUCDISC: discrimination of nuclear localization signals
pat4: RPRR (4) at 281
pat7: PRLRPRR (3) at 278
bipartite: none
content of basic residues: 14.1%
NLS Score: 0.04
KDEL: ER retention motif in the C-terminus: none
ER Membrane Retention Signals: none
SKL: peroxisomal targeting signal in the C-terminus: none
PTS2: 2nd peroxisomal targeting signal: found
RLRAKLLQL at 167
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: nuclear
Reliability: 89
COIL: Lupas's algorithm to detect coiled-coil regions
total: 0 residues
Final Results (k = 9/23):
65.2%: nuclear
17.4%: mitochondrial
13.0%: extracellular, including cell wall
4.3%: cytoplasmic
>> prediction for CG57054-03 is nuc (k = 23)

A search of the NOV14a protein against the Geneseq database, a proprietary database that contains sequences published in patents and patent publication, yielded several homologous proteins shown in Table 14D.

TABLE 14D
Geneseq Results for NOV14a

| | | NOV14a | Identities/ | |
|---|---|---|---|---|
| | | Residues/ | Similarities for | |
| Geneseq | Protein/Organism/Length [Patent | Match | the Matched | Expect |
| Identifier | #, Date] | Residues | Region | Value |
| AAE34046 | WNT-10B protein - Unidentified, | 1 . . . 370 | 357/400 (89%) | 0.0 |
| | 389 aa. [WO200290992-A2, | 1 . . . 389 | 357/400 (89%) | |
| | 14 NOV. 2002] | | | |
| ABU55892 | Human WNT-10B protein - Homo | 1 . . . 370 | 357/400 (89%) | 0.0 |
| | sapiens, 389 aa. [WO200277204-A2, | 1 . . . 389 | 357/400 (89%) | |
| | 03 OCT. 2002] | | | |
| AAW08928 | Wnt-10b protein - Homo sapiens, | 1 . . . 370 | 355/400 (88%) | 0.0 |
| | 389 aa. [WO9640910-A1, | 1 . . . 389 | 355/400 (88%) | |
| | 19 DEC. 1996] | | | |
| AAR53689 | HR2 polypeptide - Homo sapiens, | 1 . . . 370 | 348/400 (87%) | 0.0 |
| | 389 aa. [WO9411510-A2, | 1 . . . 389 | 351/400 (87%) | |
| | 26 MAY 1994] | | | |
| AAY28559 | Wnt-10a polypeptide #1 -Homo | 5 . . . 370 | 224/415 (53%) | e−128 |
| | sapiens, 417 aa. [WO9938966-A1, | 14 . . . 417 | 276/415 (65%) | |
| | 05 AUG. 1999] | | | |

In a BLAST search of public sequence databases, the NOV14a protein was found to have homology to the proteins shown in the BLASTP data in Table 14E.

TABLE 14E
Public BLASTP Results for NOV14a

| | | NOV14a | Identities/ | |
|---|---|---|---|---|
| Protein | | Residues/ | Similarities for | |
| Accession | | Match | the Matched | Expect |
| Number | Protein/Organism/Length | Residues | Portion | Value |
| O00744 | Wnt-10b protein precursor (Wnt-12) - | 1 . . . 370 | 358/400 (89%) | 0.0 |
| | Homo sapiens (Human), 389 aa. | 1 . . . 389 | 358/400 (89%) | |
| P48614 | Wnt-10b protein precursor (Wnt-12) - | 1 . . . 370 | 348/400 (87%) | 0.0 |
| | Mus musculus (Mouse), 389 aa. | 1 . . . 389 | 351/400 (87%) | |
| Q9GZT5 | Wnt-10a protein precursor - Homo | 5 . . . 370 | 224/415 (53%) | e−128 |
| | sapiens (Human), 417 aa. | 14 . . . 417 | 276/415 (65%) | |
| JC7693 | soluble-type glycoprotein WNT10A - | 5 . . . 370 | 223/415 (53%) | e−127 |
| | human, 417 aa. | 14 . . . 417 | 275/415 (65%) | |
| P43446 | Wnt-10a protein precursor - | 22 . . . 370 | 210/384 (54%) | e−122 |
| | Brachydanio rerio (Zebrafish) (Danio | 72 . . . 442 | 270/384 (69%) | |
| | rerio), 442 aa. | | | |

PFam analysis predicts that the NOV14a protein contains the domains shown in the Table 14F.

TABLE 14F
Domain Analysis of NOV14a

| | | Identities/ | |
|---|---|---|---|
| Pfam | NOV14a | Similarities | |
| Domain | Match Region | for the Matched Region | Expect Value |
| wnt | 47 . . . 370 | 150/384 (39%) | 3.3e−117 |
| | | 266/384 (69%) | |

### Example 15

The NOV15 clone was analyzed, and the nucleotide and encoded polypeptide sequences are shown in Table 15A.

TABLE 15A
NOV15 Sequence Analysis

| | | |
|---|---|---|
| NOV15a, CG57431-03 | SEQ ID NO: 187 | 651 bp |
| DNA Sequence | ORF Start: ATG at 10 | ORF Stop: TAG at 445 |

ACCGCCGCTATGGTCTCCGTGCCTACCACCTGGTGCTCCGTTGCGCTAGCCCTGCTCGTGGCCCTGCATGAAG
GGAAGGGCCAGGCTGCTGCCACCCTGGAGCAGCCAGCGTCCTCATCTCATGCCCAAGGCACCCACCTTCGGCT
TCGCCGTTGCTCCTGCAGCTCCTGGCTCGACAAGGAGTGCGTCTACTTCTGCCACTTGGACATCATCTGGGTG
AACACTCCTGAACAGACAGCTCCTTACGGCCTGGGAAACCCGCCAAGACGCCGGCGCCGCTCCCTGCCAAGGC
GCTGTCAGTGCTCCAGTGCCAGGGACCCCACCTGTGCCACCTTCTGCCTTCGAAGGCCCTGGGACATTTCCAC
AGTCAAGAGCCTCTTTGCCAAGCGACAACAGGAGGCCATGCGGGAGCCTCGGTCCACACATTCCAGGTGGAGG
AAGAGATAGTGTCGTGAGCTGGAGGAACATTGGGAAGGAAGCCCGCGGGGAGAGAGGAGGAGAGAAGTGGCCA
GGGCTTGTGGACTCTCTGCCTGCTTCCTGGACCGGGGCCTTGGTCCCAGACAGCTGGACCCATTTGCCAGGAT
TGGCACAGGCTCCCTGGTGAGGGAGCCTCGTCCAAGGCACTTCTGTGTCCTCGCACTGCCCAGGGAA

| | | | |
|---|---|---|---|
| NOV15a, CG57431-03 | | | |
| Protein Sequence | SEQ ID NO: 188 | 145 aa | MW at 16563.8kD |

MVSVPTTWCSVALALLVALHEGKGQAAATLEQPASSSHAQGTHLRLRRCSCSSWLDKECVYFCHLDIIWVNTP
EQTAPYGLGNPPRRRRRSLPRRCQCSSARDPTCATFCLRRPWDISTVKSLFAKRQQEAMREPRSTHSRWRKR

| | | |
|---|---|---|
| NOV15b, CG57431-02 | SEQ ID NO: 189 | 556 bp |
| DNA Sequence | ORF Start: ATG at 11 | ORF Stop: TAG at 545 |

TACCGCCGCTATGGTTTCCGTGCCTAGCACCTGGTGCTCCGTTGCGCTAGCCCTGCTCGTGGCCCTGCATGAA
GGGAAGGGCCAGGCTGCTGCCACCCTGGAGCAGCCAGCGTCCTCATCTCATGCCCAAGGCACCCACCTTCGGC
TTCGCCGTTGCTCCTGCAGCTCCTGGCTCGACAAGGAGTGCGTCTACTTCTGCCACTTGGACATCATCTGGGT
GAACACTCCTGAACAGACAGCTCCTTACGGCCTGGGAAACCCGCCAAGACGCCGGCGCCGCTCCCTGCCAAGG
CGCTGTCAGTGCTCCAGTGCCAGGGACCCCGCCTGTGCCACCTTCTGCCTTCGAAGGCCCTGGACTGAAGCCG
GGGCAGTCCCAAGCCGGAAGTCCCCTGCAGACGTGTTCCAGACTGGCAAGACAGGGGCCACTACAGGAGAGCT
TCTCCAAAGGCTGAGGGACATTTCCACAGTCAAGAGCCTCTTTGCCAAGCGACAACAGGAGGCCATGCGGGAG
CCTCGGTCCACACATTCCAGGTGGAAGGAGAGATAGTGTCGTGAA

| | | | |
|---|---|---|---|
| NOV15b, CG57431-02 | | | |
| Protein Sequence | SEQ ID NO: 190 | 178 aa | MW at 19918.5kD |

MVSVPSTWCSVALALLVALHEGKGQAAATLEQPASSSHAQGTHLRLRRCSCSSWLDKECVYFCHLDIIWVNTP
EQTAPYGLGNPPRRRRRSLPRRCQCSSARDPACATFCLRRPWTEAGAVPSRKSPADVFQTGKTGATTGELLQR
LRDISTVKSLFAKRQQEAMREPRSTHSRWKER

| | | |
|---|---|---|
| NOV15c, CG57431-01 | SEQ ID NO: 191 | 668 bp |
| DNA Sequence | ORF Start: ATG at 40 | ORF Stop: TAG at 574 |

CTCCCTGCTCCAGTCCAGCCTGCGCGCTCCACCGCCGCTATGGTTTCCGTGCCTACCACCTGGTGCTCCGTTG
CGCTAGCCCTGCTCGTGGCCCTGCATGAAGGGAAGGGCCAGGCTGCTGCCACCCTGGAGCAGCCAGCGTCCTC
ATCTCATGCCCAAGGCACCCACCTTCGGCTTCGCCGTTGCTCCTGCAGCTCCTGGCTCGACAAGGAGTGCGTC
TACTTCTGCCACTTGGACATCATCTGGGTGAACACTCCTGAACAGACAGCTCCTTACGGCCTGGGAAACCCGC
CAAGACGCCGGCGCCGCTCCCTGCCAAGGCGCTGTCAGTGCTCCAGTGCCAGGGACCCCGCCTGTGCCACCTT
CTGCCTTCGAAGGCCCTGGACTGAAGCCGGGGCAGTCCCAACCCGGAAGTCCCCTGCAGACGTGTTCCAGACT
GGCAAGACAGGGGCCACTACAGGAGAGCTTCTCCAAAGGCTGAGGGACATTTCCACAGTCAAGAGCCTCTTTG
CCAAGCGACAACAGGAGGCCATGCGGGAGCCTCGGTCCACACATTCCAGGTGGAGGAAGAGATAGTGTCGTGA
GCTGGAGGAACATTGGGAAGGAAGCCCGCGGGGAGAGAGGAGGAGAGAAGTGGCCAGGGCTTGTGGACTCTCC
TGCTGCTTTCT

| | | | |
|---|---|---|---|
| NOV15c, CG5743 1-01 | | | |
| Protein Sequence | SEQ ID NO: 192 | 178 aa | MW at 19945.6kD |

MVSVPSTWCSVALALLVALHEGKGQAAATLEQPASSSHAQGTHLRLRRCSCSSWLDKECVYFCHLDIIWVNTP
EQTAPYGLGNPPRRRRRSLPRRCQCSSARDPACATFCLRRPWTEAGAVPSRKSPADVFQTGKTGATTGELLQR
LRDISTVKSLFAKRQQEAMREPRSTHSRWRKR

| | | |
|---|---|---|
| NOV15d, CG57431-04 | SEQ ID NO: 193 | 964 bp |
| DNA Sequence | ORF Start: ATG at 10 | ORF Stop: TAG at 322 |

ACCGCCGCTATGGTCTCCGTGCCTACCACCTGGTGCTCCGTTGCGCTAGCCCTGCTCGTGGCCCTGCATCAAG
GGAAGGGCCAGGCTGCTGCCACCCTGGAGCAGCCAGCGTCCTCATCTCATGCCCAAGGCACCCACCTTCGGCT
TCGCCGTTGCTCCTGCAGCTCCTGGCTCGACAAGGAGTGCGTCTACTTCTGCCACTTGGACATCATCTGGGTG
AACACTCCTGACGACATTTCCACAGTCAAGAGCCTCTTTGCCAAGCCACAACAGGAGGCCATGCGGGAGCCTC
GGTCCACACATTCCAGGTGGAGGAAGAGATAGTGTCGTGAGCTGGAGGAACATTGGGAAGGAAGCCCGCGGGG
AGAGAGGAGGAGAGAAGTGGCCGGGGCTTGTGGACTCTCTGCCTGCTTCCTGGACCGGGGCCTTGGTCCCAGA
CAGCTGGACCCATTTGCCAGGATTGGCACAAGCTCCCTGGTGAGGGAGCCTCGTCCAAGGCAGTTCTGTGTCC
TCGCACTGCCCAGGGAAGCCCTCGGCCTCCAGACTGCGGAGCAGCCTCCAGTGCTGGCTGCTGGCCCACAGCT
CTGCTGGAAGAACTGCATGGGGAGTACATTCATCTGGAGGCTGCGTCCTGAGGAGTGTCCTGTCTGCTGGGCT
ACAAACCAGGAGCGACCGTGCAGCCACGAACACGCATGCCTCAGCCAGCCCCGGAGGCTGGATGGCTCCCCTG
AGGCTGGCATCCTGGCTGGCTGTGTCCTCTCCAGCTTTCCCTCCCCAGAGTTCTTGCACCCTCATTCCCTCGG
GACCCTCCCAGTGAGAAGGGCCTGCTCTGCTTTTCCTGTCTGTATATAACTTATTTGCCCTAAGAACTTTGAG
AATCCCAATTATTTATTTTAATGTATTTTTTAGACCCTCTATTTACCTGCGAACTTGTGTTTATAATAAATGA
GGAAACAGAAAAAAA

| | | | |
|---|---|---|---|
| NOV15d, CG57431-04 | | | |
| Protein Sequence | SEQ ID NO: 194 | 104 aa | MW at 11793.4kD |

MVSVPTTWCSVALALLVALHEGKGQAAATLEQPASSSHAQGTHLRLRRCSCSSWLDKECVYFCHLDIIWVNTP
EDISTVKSLFAKRQQEAMREPRSTHSRWRKR

A ClustalW comparison of the above protein sequences yields the following sequence alignment shown in Table 15B.

TABLE 15B
Comparison of the NOV15 protein sequences.

| | |
|---|---|
| NOV15a | MVSVPTTWCSVALALLVALHEGKGQAAATLEQPASSSHAQGTHLRLRRCSCSSWLDKECV |
| NOV15b | MVSVPSTWCSVALALLVALHEGKGQAAATLEQPASSSHAQGTHLRLRRCSCSSWLDKECV |
| NOV15c | MVSVPSTWCSVALALLVALHEGKGQAAATLEQPASSSHAQGTHLRLRRCSCSSWLDKECV |
| NOV15d | MVSVPTTWCSVALALLVALHEGKGQAAATLEQPASSSHAQGTHLRLRRCSCSSWLDKECV |
| NOV15a | YFCHLDIIWVNTPEQTAPYGLGNPPRRRRR------------------------------ |
| NOV15b | YFCHLDIIWVNTPEQTAPYGLGNPPRRRRRSLPRRCQCSSARDPACATFCLRRPWTEAGA |
| NOV15c | YFCHLDIIWVNTPEQTAPYGLGNPPRRRRRSLPRRCQCSSARDPACATFCLRRPWTEAGA |
| NOV15d | YFCHLDIIWVNT------------------------------------------------ |
| NOV15a | ---SLPRRCQCSSARDPTCATFCLRRPWDISTVKSLFAKRQQEAMREPRSTHSRWRKR |
| NOV15b | VPSRKSPADVFQTGKTGATTGELLQRLRDISTVKSLFAKRQQEAMREPRSTHSRWKER |
| NOV15c | VPSRKSPADVFQTGKTGATTGELLQRLRDISTVKSLFAKRQQEAMREPRSTHSRWRKR |
| NOV15d | --------------------------PEDISTVKSLFAKRQQEAMREPRSTHSRWRKR |
| NOV15a | (SEQ ID NO: 188) |
| NOV15b | (SEQ ID NO: 190) |
| NOV15c | (SEQ ID NO: 192) |
| NOV15d | (SEQ ID NO: 194) |

Further analysis of the NOV15a protein yielded the following properties shown in Table 15C.

TABLE 15C
Protein Sequence Properties NOV15a

| SignalP | Cleavage site between residues 25 and 26 | | | |
|---|---|---|---|---|
| analysis: | | | | |
| PSORT II | PSG: a new signal peptide prediction method | | | |
| analysis: | N-region: length 0; pos. chg 0; neg. chg 0 | | | |
| | H-region: length 20; peak value 9.73 | | | |
| | PSG score: 5.33 | | | |
| | GvH: von Heijne's method for signal seq. recognition | | | |
| | GvH score (threshold: −2.1): −1.24 | | | |
| | possible cleavage site: between 24 and 25 | | | |
| | >>> Seems to have a cleavable signal peptide (1 to 24) | | | |
| | ALOM: Klein et al's method for TM region allocation | | | |
| | Init position for calculation: 25 | | | |
| | Tentative number of TMS(s) for the threshold 0.5: 0 | | | |
| | number of TMS(s) . . . fixed | | | |
| | PERIPHERAL | Likelihood = 7.85 (at 54) | | |
| | ALOM score: 7.85 (number of TMSs: 0) | | | |
| | MTOP: Prediction of membrane topology (Hartmann et al.) | | | |
| | Center position for calculation: 12 | | | |
| | Charge difference: −1.5 C(−0.5)-N(1.0) | | | |
| | N >= C: N-terminal side will be inside | | | |
| | MITDISC: discrimination of mitochondrial targeting seq | | | |
| | R content: | 0 | Hyd Moment(75): | 1.71 |
| | Hyd Moment(95): | 1.49 | G content: | 0 |
| | D/E content: | 1 | S/T content: | 4 |

Score: −4.94
Gavel: prediction of cleavage sites for mitochondrial preseq
cleavage site motif not found
NUCDISC: discrimination of nuclear localization signals
pat4: PRRR (4) at 85
pat4: RRRR (5) at 86
pat4: RRRR (5) at 87
pat7: PPRRRRR (5) at 84
pat7: PRRRRRS (5) at 85
bipartite: none
content of basic residues: 16.6%
NLS Score: 1.27
KDEL: ER retention motif in the C-terminus: none
ER Membrane Retention Signals:
KKXX-like motif in the C-terminus: RWRK
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: nuclear
Reliability: 94.1
COIL: Lupas's algorithm to detect coiled-coil regions
total: 0 residues
Final Results (k = 9/23):
55.6%: extracellular, including cell wall
33.3%: nuclear
11.1%: cytoplasmic
>> prediction for CG57431-03 is exc (k = 9)

A search of the NOV15a protein against the Geneseq database, a proprietary database that contains sequences published in patents and patent publication, yielded several homologous proteins shown in Table 15D.

TABLE 15D
Geneseq Results for NOV15a

| | | NOV15a | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| AAE15748 | Human endothelin 2 (EDN2) - Homo | 1 . . . 145 | 144/178 (80%) | 3e−79 |
| | sapiens, 178 aa. [WO200190118-A2, | 1 . . . 178 | 144/178 (80%) | |
| | 29 NOV. 2001] | | | |
| AAR23784 | Precursor ET-2 sequence - Homo | 1 . . . 145 | 144/178 (80%) | 3e−79 |
| | sapiens, 178 aa. [EP484017-A, | 1 . . . 178 | 144/178 (80%) | |
| | 06 MAY 1992] | | | |
| AAR20231 | Human endothelin-2 vasoconstrictor | 1 . . . 145 | 144/178 (80%) | 3e−79 |
| | peptide - Homo sapiens, 178 aa. | 1 . . . 178 | 144/178 (80%) | |
| | [EP468337-A, 29 JAN. 1992] | | | |
| AAR60320 | Pre-pro-vasoactive intestinal | 6 . . . 120 | 89/115 (77%) | 3e−51 |
| | contractor protein - Mus musculus, | 3 . . . 117 | 99/115 (85%) | |
| | 160 aa. [JP06169774-A, | | | |
| | 21 JUN. 1994] | | | |
| AAR60319 | Pre-pro-vasoactive intestinal | 6 . . . 120 | 89/115 (77%) | 3e−51 |
| | contractor protein - Mus musculus, | 3 . . . 117 | 99/115 (85%) | |
| | 160 aa. [JP06169774-A, | | | |
| | 21 JUN. 1994] | | | |

In a BLAST search of public sequence databases, the NOV15a protein was found to have homology to the proteins shown in the BLASTP data in Table 15E.

TABLE 15E
Public BLASTP Results for NOV15a

| | | NOV15a | Identities/ | |
|---|---|---|---|---|
| Protein | | Residues/ | Similarities for | |
| Accession | | Match | the Matched | Expect |
| Number | Protein/Organism/Length | Residues | Portion | Value |
| P20800 | Endothelin-2 precursor (ET-2) - | 1 . . . 145 | 144/178 (80%) | 8e−79 |
| | Homo sapiens (Human), 178 aa. | 1 . . . 178 | 144/178 (80%) | |
| BAC54893 | Preproendothelin-2 - Equus caballus | 1 . . . 145 | 116/178 (65%) | 2e−60 |
| | (Horse), 178 aa. | 1 . . . 178 | 123/178 (68%) | |
| Q8MJW9 | Preproendothelin-2 - Mustela putorius | 1 . . . 144 | 108/177 (61%) | 2e−56 |
| | furo (Ferret), 178 aa. | 1 . . . 177 | 120/177 (67%) | |
| P23943 | Endothelin-2 precursor (ET-2) | 4 . . . 145 | 103/175 (58%) | 4e−53 |
| | (Vasoactive intestinal contractor) | 2 . . . 176 | 114/175 (64%) | |
| | (VIC) - Rattus norvegicus (Rat), 176 | | | |
| | aa. | | | |
| S17194 | endothelin 2 precursor - mouse, 160 | 6 . . . 120 | 89/115 (77%) | 8e−51 |
| | aa. | 3 . . . 117 | 99/115 (85%) | |

PFam analysis predicts that the NOV15a protein contains the domains shown in the Table 15F.

TABLE 15F
Domain Analysis of NOV15a

| | | Identities/ | |
|---|---|---|---|
| | NOV15a | Similarities | Expect |
| Pfam Domain | Match Region | for the Matched Region | Value |
| endothelin | 44 . . . 74 | 28/31 (90%) | 5.4e−19 |
| | | 29/31 (94%) | |

### Example 16

The NOV16 clone was analyzed, and the nucleotide and encoded polypeptide sequences are shown in Table 16A.

TABLE 16A
NOV16 Sequence Analysis

| | | |
|---|---|---|
| NOV16a, CG59253-01 | SEQ ID NO: 195 | 1894 bp |
| DNA Sequence | ORF Start: ATG at 46 | ORF Stop: TAG at 1474 |

TGGCATTTCTGAGCAGGGGCCACCCTGACTTCACCTTGGCCCACCATGAGGGTCTTCCTGCTTTGTGCCTACA
TACTGCTGCTGATGGTTTCCCAGTTGAGGGCAGTCAGCTTTCCTGAAGATGATGAACCCCTTAATACTGTCGA
CTATCACTATTCAAGGCAATATCCGGTTTTTAGAGGACGCCCTTCAGGCAATGAATCGCAGCACAGGCTGGAC
TTTCAGCTGATGTTGAAAATTCGAGACACACTTTATATTGCTGGCAGGGATCAAGTTTATACAGTAAACTTAA
ATGAAATGCCCAAAACAGAAGTAATACCCAACAAGAAACTGACATGGCGATCAAGACAACAGGATCGAGAAAA
CTGTGCTATGAAAGGCAAGCATAAAGATGAATGCCACAACTTTATCAAAGTATTTGTTCCAAGAAACGATGAG
ATGGTTTTTGTTTGTGGTACCAATGCATTCAATCCCATGTGTAGATACTACAGGTTGAGTACCTTAGAATATG
ATGGGGAAGAAATTAGTGGCCTGGCAAGATGCCCATTTGATGCCAGACAAACCAATGTTGCCCTCTTTGCTGA
TGGGAAGCTGTATTCTGCCACAGTGGCTGACTTCTTGGCCAGCGATGCCGTTATTTATCGAAGCATGGGTGAT
GGATCTGCCCTTCGCACAATAAAATATGATTCCAAATGGATAAAAGAGCCACACTTTCTTCATGCCATAGAAT
ATGGAAACTATGTCTATTTCTTCTTTCGAGAAATCGCTGTCGAACATAATAATTTAGGCAAGGCTGTGTATTC
CCGCGTGGCCCGCATATGTAAAAACGACATGGGTGGTTCCCAGCGGGTCCTGGAGAAACACTGGACTTCATTT
CTAAACGCTCGGCTGAACTGTTCTGTCCCTGGAGATTCGTTTTTCTACTTTGATGTTCTGCAGTCTATTACAG
ACATAATACAAATCAATGGCATCCCCACTGTGGTCGGGGTGTTTACCACGCAGCTCAATAGCATCCCTGGTTC
TGCTGTCTGTGCATTTAGCATGGATGACATTGAAAAAGTATTCAAAGGACGGTTTAAGGAACAGAAAACTCCA
GATTCTGTTTGGACAGCAGTTCCCGAAGACAAAGTGCCAAAGCCAAGGCCTGGCTGTTGTGCAAAACACGGCC
TTGCCGAAGCTTATAAAACCTCCATCGATTTCCCGGATGAAACTCTGTCATTCATCAAATCTCATCCCCTGAT
GGACTCTGCCGTTCCACCCATTGCCGATGAGCCCTGGTTCACAAAGACTCGGGTCAGGTACAGACTGACGGCC
ATCTCAGTGGACCATTCAGCCGGACCCTACCAGAACTACACAGTCATCTTTGTTGGCTCTGAAGCTGGCATGG
TACTTAAAGTTCTGGCAAAGACCAGTCCTTTCTCTTTGAACGACAGCGTATTACTGGAAGAGATTGAAGCCTA
CAACCATGCAAAGTAGGTATATGTTACGAGAACGCCCTTCAGCACTGCTCAAAAATTTTCGGCATGTATTTCA
TCTAGTCATGTCCTTTTGGTCCTCTAAATTAGCAGTGGTTTGGCATAATAGTGTTTTGTGTTTTTTTTCTCAT
TGAAATAAATCTTGGGTTTGTTTTTTTCCCGAGCCTGCTAGGGCGAGGGGGGTGAATGGTTGATCAGTTTAAA
AATAATGCAGCCCTTGTTTTTCACCTCTAGAATATGAGAACATTTTAACAGCACCTCTCTTATCTTGCAGATA
TATTCCAAGATGCTACATGCAGCAGACAGCTGTGAGCTTGCATACACACACACACAAATATACATGCACATAC
ATACACAGAATGTAGTACTAGTTAAGTATTTCCTTCCTATCTTTAATAAGTAAGAGAATATTTAGACCA

| | | | |
|---|---|---|---|
| NOV16a, CG59253-01 | | | |
| Protein Sequence | SEQ ID NO: 196 | 476 aa | MW at 54216.4kD |

MRVFLLCAYILLLMVSQLRAVSFPEDDEPLNTVDYHYSRQYPVFRGRPSGNESQHRLDFQLMLKIRDTLYIAG
RDQVYTVNLNEMPKTEVIPNKKLTWRSRQQDRENCAMKGKHKDECHNFIKVFVPRNDEMVFVCGTNAFNPMCR
YYRLSTLEYDGEEISGLARCPFDARQTNVALFADGKLYSATVADFLASDAVIYRSMGDGSALRTIKYDSKWIK
EPHFLHAIEYGNYVYFFFREIAVEHNNLGKAVYSRVARICKNDMGGSQRVLEKHWTSFLKARLNCSVPGDSFF
YFDVLQSITDIIQINGIPTVVGVFTTQLNSIPGSAVCAFSMDDIEKVFKGRFKEQKTPDSVWTAVPEDKVPKP
RPGCCAKHGLAEAYKTSIDFPDETLSFIKSHPLMDSAVPPIADEPWFTKTRVRYRLTAISVDHSAGPYQNYTV
IFVGSEAGMVLKVLAKTSPFSLNDSVLLEEIEAYNHAK

| | | |
|---|---|---|
| NOV16b, 194877881 | SEQ ID NO: 197 | 1383 bp |
| DNA Sequence | ORF Start: at 1 | ORF Stop: end of sequence |

GGATCCGTCAGCTTTCCTGAAGATGATGAACCCCTTAATACTGTCGACTATCACTATTCAAGGCAATATCCGG
TTTTTAGAGGACGCCCTTCAGGCAATGAATCGCAGCACAGGCTGGACTTTCAGCTGATGTTGAAAATTCGAGA
CACACTTTATATTGCTCGCAGGGATCAAGTTTATACAGTAAACTTAAATGAAATGCCCAAAACAGAAGTAATA
CCCAACAAGAAACTGACATGGCGATCAAGACAACAGGATCGAGAAAACTGTGCTATGAAAGGCAAGCATAAAG
ATGAATGCCACAACTTTATCAAAGTATTTGTTCCAAGAAACGATGAGATGGTTTTTGTTTGTGGTACCAATGC
ATTCAATCCCATGTGTAGATACTACAGGTTGAGTACCTTAGAATATGATGGGGAAGAAATTAGTGGCCTGGCA
AGATGCCCATTTGATGCCAGACAAACCAATGTTGCCCTCTTTGCTGATGGGAAGCTGTATTCTGCCACAGTGG
CTGACTTCTTGGCCAGCGATGCCGTTATTTATCGAAGCATGGGTGATGGATCTGCCCTTCGCACAATAAAATA
TGATTCCAAATGGATAAAAGAGCCACACTTTCTTCATGCCATAGAATATGGAAACTATGTCTATTTCTTCTTT
CGAGAAATCGCTGTCGAACATAATAATTTAGGCAAGGCTGTGTATTCCCGCGTGGCCCGCATATGTAAAAACG
ACATGGGTGGTTCCCAGCGGGTCCTGGAGAAACACTGGACTTCATTTCTAAAGGCTCGGCTGAACTGTTCTGT
CCCTGGAGATTCGTTTTTCTACTTTGATGTTCTGCAGTCTATTACAGACATAATACAAATCAATGGCATCCCC
ACTGTGGTCGGGGTGTTTACCACCCAGCTCAATAGCATCCCTGGTTCTGCTGTCTGTGCATTTAGCATGGATG
ACATTGAAAAAGTATTCAAAGGACGGTTTAAGGAACAGAAAACTCCAGATTCTGTTTGGACAGCAGTTCCCGA
AGACAAAGTGCCAAAGCCAAGGCCTGGCTGTTGTGCAAAACACGGCCTTGCCGAAGCTTATAAAACCTCCATC
GATTTCCCGGATGAAACTCTGTCATTCATCAAATCTCATCCCCTGATGGACTCTGCCGTTCCACCCATTGCCG
ATGAGCCCTGGTTCACAAAGACTCGGGTCAGGTACAGACTGACGGCCATCTCAGTGGACCATTCAGCCGGACC
CTACCAGAACTACACAGTCATCTTTGTTGGCTCTGAAGCTGGCATGGTACTTAAAGTTCTGGCAAAGACCAGT
CCTTTCTCTTTGAACGACAGCGTATTACTGGAAGAGATTGAAGCCTACAACCATGCAAAGTGCCTCGAG

| | | | |
|---|---|---|---|
| NOV16b, 194877881 | | | |
| Protein Sequence | SEQ ID NO: 198 | 461 aa | MW at 52370.0kD |

GSVSFPEDDEPLNTVDYHYSRQYPVFRGRPSGNESQHRLDFQLMLKIRDTLYIAGRDQVYTVNLNEMPKTEVI
PNKKLTWRSRQQDRENCAMKGKHKDECHNFIKVFVPRNDEMVFVCGTNAFNPMCRYYRLSTLEYDGEEISGLA
RCPFDARQTNVALFADGKLYSATVADFLASDAVIYRSMGDGSALRTIKYDSKWIKEPHFLHAIEYGNYVYFFF
REIAVEHNNLGKAVYSRVARICKNDMGGSQRVLEKHWTSFLKARLNCSVPGDSFFYFDVLQSITDIIQINGIP
TVVGVFTTQLNSIPGSAVCAFSMDDIEKVFKGRFKEQKTPDSVWTAVPEDKVPKPRPGCCAKHGLAEAYKTSI
DFPDETLSFIKSHPLMDSAVPPIADEPWFTKTRVRYRLTAISVDHSAGPYQNYTVIFVGSEAGMVLKVLAKTS
PFSLNDSVLLEEIEAYNHAKCLE

| | | |
|---|---|---|
| NOV16c, CG59253-02 | SEQ ID NO: 199 | 3205 bp |
| DNA Sequence | ORF Start: ATG at 46 | ORF Stop: TAG at 3151 |

TGGCATTTCTGAGCAGGGGCCACCCTGACTTCACCTTGGCCCACCATGAGGGTCTTCCTGCTTTGTGCCTACA
TACTGCTGCTGATGGTTTCCCAGTTGAGGGCAGTCAGCTTTCCTGAAGATGATGAACCCCTTAATACTGTCGA
CTATCACTGTAAGTCGTCTAGGCAATATCCGGTTTTTAGAGGACGCCCTTCAGGCAATGAATCGCAGCACAGG
CTGGACTTTCAGCTGATGTTGAAAATTCGAGACACACTTTATATTGCTGGCAGGGATCAAGTTTATACAGTAA
ACTTAAATGAAATGCCCAAAACAGAAGTAATATGGCAACAGAAACTGACATGGCGATCAAGACAACAGGATCG
AGAAAACTGTGCTATGAAAGGCAAGCATAAAGATGAATGCCACAACTTTATCAAAGTATTTGTTCCAAGAAAC
GATGAGATGGTTTTTGTTTGTGGTACCAATGCATTCAATCCCATGTGTAGATACTACAGGGTAAGTACCTTAG
AATATGATGGGGAAGAAATTAGTGGCCTGGCAAGATGCCCATTTGATGCCAGACAAACCAATGTTGCCCTCTT
TGCTGATGGGAAGCTGTATTCTGCCACAGTGGCTGACTTCTTGGCCAGCGATGCCGTTATTTATCGAAGCATG
GGTGATGGATCTGCCCTTCGCACAATAAAATATGATTCCAAATGGATAAAAGAGCCACACTTTCTTCATGCCA
TAGAATATGGAAACTATGTCTATTTCTTCTTTCGAGAAATCGCTGTCGAACATAATAATTTAGGCAAGGCTGT
GTATTCCCGCGTGGCCCGCATATGTAAAAACGACATGGGTGGTTCCCAGCGGGTCCTGGAGAAACACTGGACT
TCATTTCTAAAGGCTCGGCTGAACTGTTCTGTCCCTGGAGATTCGTTTTTCTACTTTGATGTTCTGCAGTCTA
TTACAGACATAATACAAATCAATGGCATCCCCACTGTGGTCGGGGTGTTTACCACGCAGCTCAATAGCATCCC
TGGTTCTGCTGTCTGTGCATTTAGCATGGATGACATTGAAAAAGTATTCAAAGGACGGTTTAAGGAACAGAAA
ACTCCAGATTCTGTTTGGACAGCAGTTCCCGAAGACAAAGTGCCAAAGCCAAGGCCTGGCTGTTGTGCAAAAC
ACGGCCTTGCCGAAGCTTATAAAACCTCCATCGATTTCCCGGATGAAACTCTGTCATTCATCAAATCTCATCC
CCTGATGGACTCTGCCGTTCCACCCATTGCCGATGAGCCCTGGTTCACAAAGACTCGGGTCAGGTACAGACTG
ACGGCCATCTCAGTGGACCATTCAGCCGGACCCTACCAGAACTACACAGTCATCTTTGTTGGCTCTGAAGCTG
GCATGGTACTTAAAGTTCTGGCAAAGACCAGTCCTTTCTCTTTGAACGACAGCGTATTACTGGAAGAGATTGA
AGCCTACAACCATGCAAAGTGCAGTGCTGAGAATGAGGAAGACAAAAAGGTCATCTCATTACAGTTGGATAAA
GATCACCACGCTTTATATGTGGCGTTCTCTAGCTGCATTATCCGCATCCCCCTCAGTCGCTGTGAGCGTTATG
GATCATGTAAAAAGTCTTGTATTGCATCTCGTCACCCGTATTGTCGCTGGTTAAGCCAGGCATCCTGTGCTAG
AGTGACCCCAAACCACAGTGCTGAAGGATATGAACAAGACACAGAATTCGGCAACACAGCTCATCTAGGGGAC
TGCCATGCATATGAACCATATGAAGGTCGTGTTGGCTCACTGAAAGCCATTTGCTATTTATTATTATTTTTAA
AAAGCACCTTATTCACATTGTCCCATGTGTCTATTTCAGGTGTACGATGGGAAGTCCAGTCTGGAGAGTCCAA
CCAGATGGTCCACATGAATCTCCTCATCACCTGTGTCTTTGCTGCTTTTGTTTTCGCGGCATTCATTGCAGGT
GTGGCAGTATACTGCTATCGAGACATGTTTGTTCGGAAAAACAGAAAGATCCATAAAGATGCAGAGTCCGCCC
AGTCATGCACAGACTCCAGTGGAAGTTTTGCCAAACTGAATGGTCTCTTTGACAGCCCTGTCAAGGAATACCA
ACAGAATATTGATTCTCCTAAACTGTATAGTAACCTGCTAACCAGTCGGAAAGAGCTACCACCCAATGGAGAT
ACTAAATCCATGGTAATGGACCATCGAGGGCAACCTCCAGAGTTGGCTGCTCTTCCTACTCCTGAGTCTACAC
CCGTGCTTCACCAGAAGACCCTGCAGGCCATGAAGAGCCACTCAGAAAAGGCCCATGGCCATGGAGCTTCAAG
GAAAGAAACCCCTCAGTTTTTTCCGTCTAGTCCGCCACCTCATTCCCCATTAAGTCATGGGCATATCCCCAGT
GCCATTGTTCTTCCAAATGCTACCCATGACTACAACACGTCTTTCTCAAACTCCAATGCTCACAAAGCTGAAA
AGAAGCTTCAAAACATTGATCACCCTCTCACAAAGTCATCCAGTAAGAGAGATCACCCGCGTTCTGTTGATTC
CAGAAATACCCTCAATGATCTCCTGAAGCATCTGAATGACCCAAATAGTAACCCCAAAGCCATCATGGGAGAC
ATCCAGATGGCACACCAGAACTTAATGCTGGATCCCATGGGATCGATGTCTGAGGTCCCACCTAAAGTCCCTA
ACCGGGAGGCATCGCTATACTCCCCTCCTTCAACTCTCCCCAGAAATAGCCCAACCAAGCGAGTGGATGTCCC
CACCACTCCTGGAGTCCCAATGACTTCTCTGGAAAGACAAAGAGGTTATCACAAAAATTCCTCCCAGAGGCAC
TCTATATCTGCTATGCCTAAAAACTTAAACTCACCAAATGGTGTTTTGTTATCCAGACAGCCTAGTATGAACC
GTGGAGGATATATGCCCACCCCCACTGGGGCGAAGGTGGACTATATTCAGGGAACACCAGTGAGTGTTCATCT
GCAGCCTTCCCTCTCCAGACAGAGCAGCTACACCAGTAATGGCACTCTTCCTAGGACGGGACTAAAGAGGACG
CCGTCCTTAAAACCTGACGTGCCACCAAAGCCTTCCTTTGTTCCTCAAACCCCATCTGTCAGACCACTGAACA
AATACACATACTAGGCCTCAAGTGTGCTATTCCCATGTGGCTTTATCCTGTCCGTGTTGTTGAGAG

| | | | |
|---|---|---|---|
| NOV16c, CG59253-02 | | | |
| Protein Sequence | SEQ ID NO: 200 | 1035 aa | MW at 115912.6kD |

MRVFLLCAYILLLMVSQLRAVSFPEDDEPLNTVDYHCKSSRQYPVFRGRPSGNESQHRLDFQLMLKIRDTLYI
AGRDQVYTVNLNEMPKTEVIWQQKLTWRSRQQDRENCANKGKHKDECHNFIKVFVPRNDEMVFVCGTNAFNPM
CRYYRVSTLEYDGEEISGLARCPFDARQTNVALFADGKLYSATVADFLASDAVIYRSMGDGSALRTIKYDSKW
IKEPHFLHAIEYGNYVYFFFREIAVEHNNLGKAVYSRVARICKNDMGGSQRVLEKHWTSFLKARLNCSVPGDS
FFYFDVLQSITDIIQINGIPTVVGVFTTQLNSIPGSAVCAFSMDDIEKVFKGRFKEQKTPDSVWTAVPEDKVP
KPRPGCCAKHGLAEAYKTSIDFPDETLSFIKSHPLMDSAVPPIADEPWFTKTRVRYRLTAISVDHSAGPYQNY
TVIFVGSEAGMVLKVLAKTSPFSLNDSVLLEEIEAYNHAKCSAENEEDKKVISLQLDKDHHALYVAFSSCIIR
IPLSRCERYGSCKKSCIASRDPYCGWLSQGSCGRVTPNHSAEGYEQDTEFGNTAHLGDCHAYEPYEGRVGSLK
AICYLLLFLKSTLFTLSHVSISGVRWEVQSGESNQMVHMNVLITCVFAAFVLGAFIAGVAVYCYRDMFVRKNR
KIHKDAESAQSCTDSSGSFAKLNGLFDSPVKEYQQNIDSPKLYSNLLTSRKELPPNGDTKSMVMDHRGQPPEL
AALPTPESTPVLHQKTLQAMKSHSEKAHGHGASRKETPQFFPSSPPPHSPLSHGHIPSAIVLPNATHDYNTSF
SNSNAHKAEKKLQNIDHPLTKSSSKRDHRRSVDSRNTLNDLLKHLNDPNSNPKAIMGDIQMAHQNLMLDPMGS
MSEVPPKVPNREASLYSPPSTLPRNSPTKRVDVPTTPGVPMTSLERQRGYHKNSSQRHSISAMPKNLNSPNGV
LLSRQPSMNRGGYMPTPTGAKVDYIQGTPVSVHLQPSLSRQSSYTSNGTLPRTGLKRTPSLKPDVPPKPSFVP
QTPSVRPLNKYTY

| | | |
|---|---|---|
| NOV16d, 191815765 | SEQ ID NO: 201 | 1713 bp |
| DNA Sequence | ORF Start: at 1 | ORF Stop: end of sequence |

GGATCCGTCAGCTTTCCTGAAGATGATGAACCCCTTAATACTGTCGACTATCACTATTCAAGGCAATATCCGG
TTTTTAGAGGACGCCCTTCAGGCAATGAATCGCAGCACAGGCTGGACTTTCAGCTGATGTTGAAAATTCGAGA
CACACTTTATATTGCTGGCGGGGATCAAGTTTATACAGTAAACTTAAATGAAATGCCCAAAACAGAAGTAATA
CCCAACAAGAAACTGACATGGCGATCAAGACAACAGGATCGAGAAAACTGTGCTATGAAAGGCAAGCATAAAG
ATGAATGCCACAACTTTATCAAAGTATTTGTTCCAAGAAACGATGAGATGGTTTTTGTTTGTGGTACCAATGC
ATTCAATCCCATGTGTAGATACTACAGGTTGAGTACCTTACAATATGATGGGGAAGAAATTAGTGGCCTGGCA
AGATGCCCATTTGATGCCAGACAAACCAATGTTGCCCTCTTTGCTGATGGGAAGCTGTATTCTGCCACAGTGG
CTGACTTCTTGGCCAGCGATGCCGTTATTTATCGAAGCATGGGTGATGGATCTGCCCTTCGCACAATAAAATA
TGATTCCAAATGGATAAAAGAGCCACACTTTCTTCATGCCATAGAATATGGAAACTATGTCTATTTCTTCTTT
CGAGAAATCGCTGTCGAACATAATAATTTAGGCAAGGCTGTGTATTCCCGCGTGGCCCGCATATGTAAAAACG
ACATCGGTGGTTCCCAGCGGGTCCTGGAGAAACACTGGACTTCATTTCTAAAGGCTCGGCTGAACTGTTCTGT
CCCTGGAGATTCGTTTTTCTACTTTGATGTTCTGCAGTCTATTACAGACATAATACAAATCAATGGCATCCCC
ACTGTGGTCGGGGTGTTTACCACGCAGCTCAATAGCATCCCTGGTTCTCCTGTCTGTGCATTTAGCATGGATG
ACATTGAAAAAGTATTCAAAGGACGGTTTAAGGAACAGAAAACTCCAGATTCTGTTTGGACAGCAGTTCCCGA
AGACAAAGTGCCAAAGCCAAGGCCTGGCTGTTGTGCAAAACACGGCCTTGCCGAAGCTTATAAAACCTCCATC
GATTTCCCGGATGAAACTCTGTCGTTCATCAAATCTCATCCCCTGATGGACTCTGCCGTTCCACCCATTGCCG
ATGAGCCCTGGTTCACAAAGACTCGGGTCAGGTACAGACTGACGGCCATCTCAGTGGACCATTCAGCCGGACC
CTACCAGAACTACACAGTCATCTTTGTTGGCTCTGAAGCTGGCATGGTACTTAAAGTTCTGGCAAAGACCAGT
CCTTTCTCTTTGAACGACAGCGTATTACTGGAAGAGATTGAAGCCTACAACCATGCAAGTGCCAATGCTGAGA
ATGAGGAAGACAAAAAGGTCATCTCATTACAGTTGGATAAAGATCACCACGCTTTATATGTGGCGTTCTCTAG
CTGCATTATCCGCATCCCCCTCAGTCGCTGTGAGCGTTATGGATCATGTAAAAAGTCTTGTATTGCATCTCGT
GACCCGTATTGTGGCTGGTTAAGCCAGGGATCCTGTGGTAGAGTGACCCCAGGGATGCTTGCTGAAGGATATG
AACAAGACACAGAATTCGGCAACACAGCTCATCTAGGGGACTGCCATGGTGTACGATGGGAAGTCCAGTCTGG
AGAGTCCAACCAGATGGTCCACATGAATCTCGAG

| | | | |
|---|---|---|---|
| NOV16d, 191815765 | | | |
| Protein Sequence | SEQ ID NO: 202 | 571 aa | MW at 64535.4kD |

GSVSFPEDDEPLNTVDYHYSRQYPVFRGRPSGNESQHRLDFQLMLKIRDTLYIAGGDQVYTVNLNEMPKTEVI
PNKKLTWRSRQQDRENCAMKGKHKDECHNFIKVFVPRNDEMVFVCGTNAFNPMCRYYRLSTLEYDGEEISGLA
RCPFDARQTNVALFADGKLYSATVADFLASDAVIYRSMGDGSALRTIKYDSKWIKEPHFLHAIEYGNYVYFFF
REIAVEHNNLGKAVYSRVARICKNDMGGSQRVLEKHWTSFLKARLNCSVPGDSFFYFDVLQSITDIIQINGIP
TVVGVFTTQLNSIPGSAVCAFSMDDIEKVFKGRFKEQKTPDSVWTAVPEDKVPKPRPGCCAKHGLAEAYKTSI
DFPDETLSFIKSHPLMDSAVPPIADEPWFTKTRVRYRLTAISVDHSAGPYQNYTVTFVGSEAGMVLKVLAKTS
PFSLNDSVLLEEIEAYNHAKCNAENEEDKKVISLQLDKDHHALYVAFSSCIIRIPLSRCERYGSCKKSCIASR
DPYCGWLSQGSCGRVTPGMLAEGYEQDTEFGNTAHLGDCHGVRWEVQSGESNQMVHMNLE

| | | |
|---|---|---|
| NOV16e, CG59253-03 | SEQ ID NO: 203 | 1383 bp |
| DNA Sequence | ORF Start: at 7 | ORF Stop: at 1375 |

GGATCCGTCAGCTTTCCTGAAGATGATGAACCCCTTAATACTGTCGACTATCACTATTCAAGGCAATATCCGG
TTTTTAGAGGACGCCCTTCAGGCAATGAATCGCAGCACAGGCTGGACTTTCAGCTGATGTTGAAAATTCGAGA
CACACTTTATATTGCTGGCAGGGATCAAGTTTATACAGTAAACTTAATGAAATGCCCCAAAACAGAAGTAATA
CCCAACAAGAAACTGACATGGCGATCAAGACAACAGGATCGAGAAAACTGTGCTATGAAAGGCAAGCATAAAG
ATGAATGCCACAACTTTATCAAAGTATTTGTTCCAAGAAACGATGAGATGGTTTTTGTTTGTGGTACCAATGC
ATTCAATCCCATGTGTAGATACTACAGGTTGAGTACCTTAGAATATGATGGGGAAGAAATTAGTGGCCTGGCA
AGATGCCCATTTGATGCCAGACAAACCAATGTTGCCCTCTTTGCTGATGGGAAGCTGTATTCTGCCACAGTGG
CTGACTTCTTGGCCAGCGATGCCGTTATTTATCGAAGCATGGGTGATGGATCTGCCCTTCGCACAATAAAATA
TGATTCCAAATGGATAAAAGAGCCACACTTTCTTCATGCCATAGAATATGGAAACTATGTCTATTTCTTCTTT
CGAGAAATCGCTGTCGAACATAATAATTTAGGCAAGGCTGTGTATTCCCGCGTGGCCCGCATATGTAAAAACG
ACATGGGTGGTTCCCAGCGGGTCCTGGAGAAACACTGGACTTCATTTCTAAAGGCTCGGCTGAACTGTTCTGT
CCCTGGAGATTCGTTTTTCTACTTTGATGTTCTGCAGTCTATTACAGACATAATACAAATCAATGGCATCCCC
ACTGTGGTCGGGGTCTTTACCACGCAGCTCAATAGCATCCCTGGTTCTGCTGTCTGTGCATTTAGCATGGATG
ACATTGAAAAAGTATTCAAAGGACGGTTTAAGGAACAGAAAACTCCAGATTCTGTTTGGACAGCAGTTCCCGA
AGACAAAGTGCCAAAGCCAAGGCCTGGCTGTTGTGCAAAACACGGCCTTGCCGAAGCTTATAAAACCTCCATC
GATTTCCCGGATGAAACTCTGTCATTCATCAAATCTCATCCCCTGATGGACTCTGCCGTTCCACCCATTGCCG
ATGAGCCCTGGTTCACAAAGACTCGGGTCAGGTACAGACTGACGGCCATCTCAGTGGACCATTCAGCCGGACC
CTACCAGAACTACACAGTCATCTTTGTTGGCTCTGAAGCTGGCATGGTACTTAAAGTTCTGGCAAAGACCAGT
CCTTTCTCTTTGAACGACAGCGTATTACTGGAAGAGATTGAAGCCTACAACCATGCAAAGTGCCTCGAG

| | | | |
|---|---|---|---|
| NOV16e, CG59253-03 | | | |
| Protein Sequence | SEQ ID NO: 204 | 456 aa | MW at 51880.5kD |

VSFPEDDEPLNTVDYHYSRQYPVFRGRPSGNESQHRLDFQLMLKIRDTLYIAGRDQVYTVNLNEMPKTEVIPN
KKLTWRSRQQDRENCANKGKHKDECHNFIKVFVPRNDEMVFVCGTNAFNPMCRYYRLSTLEYDGEEISGLARC
PFDARQTNVALFADGKLYSATVADFLASDAVIYRSMGDGSALRTIKYDSKWIKEPHFLHAIEYGNYVYFFFRE
IAVEHNNLGKAVYSRVARICKNDMGGSQRVLEKHWTSFLKARLNCSVPGDSFFYFDVLQSITDIIQINGIPTV
VGVFTTQLNSIPGSAVCAFSMDDIEKVFKGRFKEQKTPDSVWTAVPEDKVPKPRPGCCAKHGLAEAYKTSIDF
PDETLSFIKSHPLMDSAVPPIADEPWFTKTRVRYRLTAISVDHSAGPYQNYTVIFVGSEAGMVLKVLAKTSPF
SLNDSVLLEEIEAYNHAK

| | | |
|---|---|---|
| NOV16f, CG59253-04 | SEQ ID NO: 205 | 1713 bp |
| DNA Sequence | ORF Start: at 7 | ORF Stop: at 1708 |

GGATCCGTCAGCTTTCCTGAAGATCATGAACCCCTTAATACTGTCGACTATCACTATTCAAGGCAATATCCGG
TTTTTAGAGGACGCCCTTCAGGCAATGAATCGCAGCACAGGCTGGACTTTCAGCTGATGTTGAAAATTCGAGA
CACACTTTATATTGCTGGCGGGGATCAAGTTTATACAGTAAACTTAAATGAAATGCCCAAAACAGAAGTAATA
CCCAACAAGAAACTGACATGGCGATCAAGACAACAGGATCGAGAAAACTGTGCTATGAAAGGCAAGCATAAAG
ATGAATGCCACAACTTTATCAAAGTATTTGTTCCAAGAAACGATGAGATGGTTTTTGTTTGTGGTACCAATGC
ATTCAATCCCATGTGTAGATACTACAGGTTGAGTACCTTAGAATATGATGGGGAAGAAATTAGTGGCCTGGCA
AGATGCCCATTTGATGCCAGACAAACCAATGTTGCCCTCTTTGCTGATGGGAAGCTCTATTCTGCCACAGTGG
CTGACTTCTTGGCCAGCGATGCCGTTATTTATCGAAGCATGGGTGATGGATCTGCCCTTCGCACAATAAAATA
TGATTCCAAATGGATAAAAGAGCCACACTTTCTTCATGCCATAGAATATGGAAACTATGTCTATTTCTTCTTT
CGAGAAATCGCTGTCGAACATAATAATTTAGGCAAGGCTGTGTATTCCCGCGTGGCCCGCATATGTAAAAACG
ACATGGGTCGTTCCCAGCGGGTCCTGGAGAAACACTGGACTTCATTTCTAAAGGCTCGGCTGAACTGTTCTGT
CCCTGGAGATTCGTTTTTCTACTTTGATGTTCTGCAGTCTATTACAGACATAATACAAATCAATGGCATCCCC
ACTGTGGTCGGGGTGTTTACCACGCAGCTCAATAGCATCCCTGGTTCTGCTGTCTGTGCATTTAGCATGGATG
ACATTGAAAAAGTATTCAAAGGACGGTTTAAGGAACAGAAAACTCCAGATTCTGTTTGGACAGCAGTTCCCGA
AGACAAAGTGCCAAAGCCAAGGCCTGGCTGTTGTGCAAAACACGGCCTTGCCGAAGCTTATAAAACCTCCATC
GATTTCCCGCATGAAACTCTGTCGTTCATCAAATCTCATCCCCTGATGGACTCTGCCGTTCCACCCATTGCCG
ATGACCCCTGGTTCACAAAGACTCGGGTCAGGTACAGACTGACGGCCATCTCAGTGGACCATTCAGCCGGACC
CTACCAGAACTACACAGTCATCTTTGTTGGCTCTGAAGCTGGCATGGTACTTAAAGTTCTGGCAAAGACCAGT
CCTTTCTCTTTGAACGACAGCGTATTACTGGAAGAGATTGAAGCCTACAACCATGCAAAGTGCAATGCTGAGA
ATGAGGAAGACAAAAAGGTCATCTCATTACAGTTGGATAAAGATCACCACGCTTTATATGTGGCGTTCTCTAG
CTGCATTATCCGCATCCCCCTCAGTCGCTGTCAGCGTTATGGATCATGTAAAAAGTCTTGTATTGCATCTCGT
GACCCGTATTGTGCCTGGTTAAGCCAGGGATCCTGTGGTAGAGTGACCCCAGGGATCCTTGCTGAAGGATATG
AACAAGACACAGAATTCGGCAACACAGCTCATCTAGGGCACTGCCATGGTGTACGATGGGAAGTCCAGTCTGG
AGAGTCCAACCAGATGGTCCACATGAATCTCGAG

| | | | |
|---|---|---|---|
| NOV16f, CG59253-04 | | | |
| Protein Sequence | SEQ ID NO: 206 | 567 aa | MW at 64149.1kD |

VSFPEDDEPLNTVDYHYSRQYPVFRGRPSGNESQHRLDFQLMLKIRDTLYIAGGDQVYTVNLNEMPKTEVIPN
KKLTWRSRQQDRENCAMKGKHKDECHNFTKVFVPRNDEMVFVCGTMAFNPMCRYYRLSTLEYDCEEISGLARC
PFDARQTNVALFADGKLYSATVADFLASDAVIYRSMGDGSALRTIKYDSKWIKEPHFLHAIEYGNYVYFFFRE
IAVENNNLGKAVYSRVARICKNDMGGSQRVLEKHWTSFLKARLNCSVPGDSFFYFDVLQSITDIIQINGIPTV
VGVFTTQLNSIPGSAVCAFSMDDIEKVFKGRFKEQKTPDSVWTAVPEDKVPKPRPGCCAKHGLAEAYKTSIDF
PDETLSFIKSHPLMDSAVPPIADEPWFTKTRVRYRLTAISVDHSAGPYQNYTVIFVGSEAGMVLKVLAKTSPF
SLNDSVLLEEIEAYNHAKCNAENEEDKKVISLQLDKDHHALYVAFSSCIIRIPLSRCERYGSCKKSCIASRDP
YCGWLSQGSCGRVTPGMLAEGYEQDTEFGNTAHLGDCHGVRWEVQSGESNQMVHMN

| | | |
|---|---|---|
| NOV16g, CG59253-05 | SEQ ID NO: 207 | 2191 bp |
| DNA Sequence | ORF Start: ATG at 46 | ORF Stop: TGA at 2182 |

TGGCATTTCTGAGCAGGGGCCACCCTGACTTCACCTTGGCCCACCATGAGGGTCTTCCTGCTTTGTGCCTACA
TACTGCTGCTGATGGTTTCCCAGTTGAGGGCAGTCAGCTTTCCTGAAGATGATGAACCCCTTAATACTGTCGA
CTATCACTGTAAGTCGTCTAGGCAATATCCGGTTTTTAGAGGACGCCCTTCAGGCAATGAATCGCAGCACAGG
CTGGACTTTCAGCTGATGTTGAAAATTCGAGACACACTTTATATTGCTGGCAGGGATCAAGTTTATACAGTAA
ACTTAAATGAAATGCCCAAAACAGAAGTAATATGGCAACAGAAACTGACATGGCGATCAAGACAACAGGATCC
AGAAAACTGTGCTATGAAAGGCAAGCATAAAGATGAATGCCACAACTTTATCAAAGTATTTGTTCCAAGAAAC
GATGAGATGGTTTTTGTTTGTGGTACCAATGCATTCAATCCCATGTGTAGATACTACAGGGTAAGTACCTTAG
AATATGATGGGGAAGAAATTAGTGGCCTCGCAAGATGCCCATTTGATGCCAGACAAACCAATGTTCCCCTCTT
TGCTGATGGGAAGCTGTATTCTGCCACAGTGCCTGACTTCTTGGCCAGCGATGCCGTTATTTATCGAAGCATC
GGTGATGGATCTGCCCTTCGCACAATAAAATATGATTCCAAATGGATAAAAGAGCCACACTTTCTTCATGCCA
TAGAATATGGAAACTATGTCTATTTCTTCTTTCGAGAAATCGCTGTCGAACATAATAATTTAGGCAAGGCTGT
GTATTCCCGCGTGGCCCGCATATGTAAAAACGACATCGGTGGTTCCCAGCGGGTCCTGGAGAAACACTGGACT
TCATTTCTAAAGGCTCGGCTGAACTGTTCTGTCCCTGGAGATTCGTTTTTCTACTTTGATGTTCTGCAGTCTA
TTACAGACATAATACAAATCAATGGCATCCCCACTGTCGTCGGGGTGTTTACAACGCAGCTCAATAGCATCCC
TGGTTCTGCTGTCTGTGCATTTAGCATGGATGACATTGAAAAAGTATTCAAAGGACGGTTTAAGGAACAGAAA
ACTCCAGATTCTGTTTGGACAGCAGTTCCCGAAGACAAAGTGCCAAAGCCAAGGCCTGGCTGTTGTGCAAAAC
ACGGCCTTGCCGAAGCTTATAAAACCTCCATCGATTTCCCGGATGAAACTCTGTCATTCATCAAATCTCATCC
CCTGATGGACTCTGCCGTTCCACCCATTGCCGATGAGCCCTGGTTCACAAAGACTCGGGTCAGGTACAGACTG
ACGGCCATCTCAGTGGACCATTCAGCCGGACCCTACCAGAACTACACAGTCATCTTTGTTGGCTCTGAAGCTG
GCATGGTACTTAAAGTTCTGGCAAAGACCAGTCCTTTCTCTTTGAACGACAGCGTATTACTGGAAGAGATTGA
AGCCTACAACCATGCAAAGTGCAGTGCTGAGAATGAGGAAGACAAAAAGGTCATCTCATTACAGTTGGATAAA
GATCACCACGCTTTATATGTGGCGTTCTCTAGCTGCATTATCCGCATCCCCCTCAGTCGCTGTGAGCGTTATG
GATCATGTAAAAAGTCTTGTATTGCATCTCGTGACCCGTATTGTGGCTGGTTAAGCCAGGGATCCTGTGGTAG
AGTGACCCCAGGGATGCTGCTGTTAACCGAAGACTTCTTTGCTTTCCATAACCACAGTGCTGAAGGATATGAA
CAAGACACAGAATTCGGCAACACAGCTCATCTAGGGGACTGCCATGAAATTTTGCCTACTTCAACTACACCAG
ATTACAAAATATTTGGCGGTCCAACATCTGGTGTACGATGGGAAGTCCAGTCTGGAGAGTCCAACCAGATGGT
CCACATGAATGTCCTCATCACCTGTGTCTTTGCTGCTTTTGTTTTGGGGGCATTCATTGCAGGTGTGCCAGTA
TACTGCTATCGAGACATGTTTGTTCGGAAAAACAGAAAGATCCATAAAGATGCAGAGTCCGCCCAGTCATGCA
CAGACTCCAGTGGAAGTTTTGCCAAACTGAATGGTCTCTTTGACAGCCCTGTCAAGGAATACCAACAGAATAT
TGATTCTCCTAAACTGTATAGTAACCTGCTAACCAGTCGGAAAGAGCACGAATTCAGCGGCCGCTGAATTCTA
G

| | | | |
|---|---|---|---|
| NOV16g, CG59253-05 | | | |
| Protein Sequence | SEQ ID NO: 208 | 712 aa | MW at 80536.8kD |

MRVFLLCAYILLLMVSQLRAVSFPEDDEPLNTVDYHCKSSRQYPVFRGRPSGNESQHRLDFQLMLKIRDTLYI
AGRDQVYTVNLNEMPKTEVIWQQKLTWRSRQQDRENCAMKGKHKDECHNFIKVFVPRNDEMVFVCGTNAFNPM
CRYYRVSTLEYDGEEISGLARCPFDARQTNVALFADGKLYSATVADFLASDAVIYRSMGDGSALRTIKYDSKW
IKEPHFLHAIEYGNYVYFFFREIAVEHNNLGKAYSRVARICKNDMGGSQRVLEKHWTSFLKARRLNCSVPGDS
FFYFDVLQSITDIIQTNGIPTVVGVFTTQLNSIPGSAVCAFSMDDIEKVFKGRFKEQKTPDSVWTAVPEDKVP
KPRPGCCAKHGLAEAYKTSIDFPDETLSFIKSHPLMDSAVPPIADEPWFTKTRVRYRLTAISVDHSAGPYQNY
TVIFVGSEAGMVLKVLAKTSPFSLNDSVLLEEIEAYNNAKCSAENEEDKKVISLQLDKDHHALYVAFSSCIIR
IPLSRCERYGSCKKSCIASRDPYCGWLSQGSCGRVTPGMLLLTEDFFAFHNHSAEGYEQDTEFGNTAHLGDCH
EILPTSTTPDYKIFGGPTSCVRWEVQSGESNQMVHMNVLITCVFAAFVLGAFIAGVAVYCYRDMFVRKNRKIH
KDAESAQSCTDSSGSFAKLNGLFDSPVKEYQQNIDSPKLYSNLLTSRKEHEFSGR

| | | |
|---|---|---|
| NOV16h, CG59253-06 | SEQ ID NO: 209 | 3196 bp |
| DNA Sequence | ORF Start: ATG at 46 | ORF Stop: TAG at 3142 |

TGGCATTTCTGAGCAGGGGCCACCCTGACTTCACCTTGGCCCACCATGAGGGTCTTCCTGCTTTGTGCCTACA
TACTGCTGCTGATGGTTTCCCAGTTGAGGGCAGTCAGCTTTCCTGAAGATGATGAACCCCTTAATACTGTCGA
CTATCACTGTAAGTCGTCTAGGCAATATCCCCTTTTTAGAGGACGCCCTTCAGGCAATGAATCGCAGCACAGG
CTGGACTTTCAGCTGATGTTGAAAATTCGAGACACACTTTATATTGCTGGCAGGGATCAAGTTTATACAGTAA
ACTTAAATGAAATGCCCAAAACAGAAGTAATATGGCAACAGAAACTGACATGGCGATCAAGACAACAGGATCG
AGAAAACTGTGCTATGAAAGGCAAGCATAAAGATGAATGCCACAACTTTATCAAAGTATTTGTTCCAAGAAAC
GATGACATGGTTTTTGTTTGTGGTACCAATGCATTCAATCCCATGTCTAGATACTACAGCGTAAGTACCTTAG
AATATGATGGGGAAGAAATTAGTGGCCTGGCAAGATGCCCATTTGATGCCAGACAAACCAATGTTGCCCTCTT
TGCTGATGGGAAGCTGTATTCTGCCACAGTGCCTGACTTCTTGGCCAGCGATGCCGTTATTTATCGAAGCATG
GGTGATGGATCTGCCCTTCGCACAATAAAATATGATTCCAAATGCATAAAAGAGCCACACTTTCTTCATGCCA
TAGAATATGGAAACTATGTCTATTTCTTCTTTCGAGAAATCGCTGTCGAACATAATAATTTAGGCAAGGCTGT
GTATTCCCGCGTGGCCCGCATATGTAAAAACGACATGGGTGGTTCCCAGCGGGTCCTGGAGAAACACTGGACT
TCATTTCTAAAGGCTCGGCTGAACTGTTCTCTCCCTGGAGATTCGTTTTTCTACTTTGATGTTCTGCAGTCTA
TTACAGACATAATACAAATCAATGGCATCCCCACTGTGGTCGGGGTGTTTACCACGCAGCTCAATAGCATCCC
TGGTTCTGCTGTCTGTGCATTTAGCATGGATGACATTGAAAAAGTATTCAAAGGACGGTTTAAGGAACAGAAA
ACTCCAGATTCTGTTTGGACAGCAGTTCCCGAAGACAAAGTGCCAAAGCCAAGGCCTGGCTGTTGTGCAAAAC
ACGGCCTTCCCGAAGCTTATAAAACCTCCATCGATTTCCCGGATGAAACTCTGTCATTCATCAAATCTCATCC
CCTGATGGACTCTGCCGTTCCACCCATTGCCGATGAGCCCTGGTTCACAAAGACTCGGGTCAGCTACAGACTG
ACGGCCATCTCAGTGGACCATTCAGCCGGACCCTACCAGAACTACACAGTCATCTTTGTTGGCTCTGAACCTG
GCATGGTACTTAAAGTTCTGGCAAAGACCAGTCCTTTCTCTTTGAACGACAGCGTATTACTGGAAGAGATTGA
AGCCTACAACCATGCAAAGTGCAGTGCTGAGAATGAGGAAGACAAAAAGGTCATCTCATTACAGTTGGATAAA
GATCACCACGCTTTATATGTGGCGTTCTCTAGCTGCATTATCCGCATCCCCCTCAGTCGCTGTGAGCGTTATG
GATCATGTAAAAAGTCTTGTATTGCATCTCGTGACCCGTATTGTGGCTGGTTAAGCCAGGGATCCTGTGGTAG
AGTGACCCCAGGGATGCTGCTGTTAACCGAAGACTTCTTTGCTTTCCATAACCACAGTGCTGAAGGATATGAA
CAAGACACAGAATTCGGCAACACAGCTCATCTAGGGGACTGCCATGAAATTTTGCCTACTTCAACTACACCAG
ATTACAAAATATTTGGCGGTCCAACATCTGGTGTACGATGGGAAGTCCAGTCTGGAGAGTCCAACCAGATGGT
CCACATGAATGTCCTCATCACCTGTGTCTTTGCTGCTTTTGTTTTGGGGGCATTCATTGCAGGTGTGGCAGTA
TACTGCTATCGAGACATGTTTGTTCGGAAAAACACAAAGATCCATAAAGATGCAGAGTCCGCCCAGTCATGCA
CAGACTCCAGTGGAAGTTTTGCCAAACTGAATGGTCTCTTTGACAGCCCTGTCAAGGAATACCAACAGAATAT
TGATTCTCCTAAACTGTATAGTAACCTGCTAACCAGTCGGAAAGAGCTACCACCCAATGGAGATTCTAAATCC
ATGGTAATGGACCATCGAGGGCAACCTCCACAGTTGGCTGCTCTTCCTACTCCTGAGTCTACACCCGTGCTTC
ACCAGAAGACCCTGCAGGCCATGAAGAGCCACTCAGAAAAGGCCCATGGCCATGGAGCTTCAAGGAAAGAAAC
CCCTCAGTTTTTTCCGTCTAGTCCGCCACCTCATTCCCCATTAAGTCATGGGCATATCCCCAGTGCCATTGTT
CTTCCAAATGCTACCCATGACTACAACACGTCTTTCTCAAACTCCAATGCTCACAAAGCTGAAAAGAAGCTTC
AAAACATTGATCACCCTCTCACAAAGTCATCCAGTAAGAGAGATCACCGGCGTTCTGTTGATTCCAGAAATAC
CCTCAATGATCTCCTGAAGCATCTGAATGACCCAAATAGTAACCCCAAAGCCATCATGGGACACATCCAGATG
GCACACCAGAACTTAATGCTGGATCCCATGGGATCGATGTCTGAGGTCCCACCTAAAGTCCCTAACCGGGAGG
CATCGCTATACTCCCCTCCTTCAACTCTCCCCAGAAATAGCCCAACCAAGCGAGTGGATGTCCCCACCACTCC
TGGAGTCCCAATGACTTCTCTGGAAAGACAAAGAGGTTATCACAAAAATTCCTCCCAGAGGCACTCTATATCT
GCTATGCCTAAAAACTTAAACTCACCAAATGGTGTTTTGTTATCCAGACAGCCTAGTATGAACCGTGGAGGAT
ATATGCCCACCCCCACTGGGGCGAAGGTGGACTATATTCAGGGAACACCAGTGAGTGTTCATCTGCAGCCTTC
CCTCTCCAGACAGAGCAGCTACACCAGTAATGGCACTCTTCCTAGGACGGGACTAAAGAGGACGCCGTCCTTA
AAACCTGACGTGCCACCAAAGCCTTCCTTTGTTCCTCAAACCCCATCTGTCAGACCACTGAACAAATACACAT
ACTAGGCCTCAAGTGTGCTATTCCCATGTGGCTTTATCCTGTCCGTGTTGTTGAGAG

| | | | |
|---|---|---|---|
| NOV16h, CG59253-06 | | | |
| Protein Sequence | SEQ ID NO: 210 | 1032 aa | MW at 115525.0kD |

MRVFLLCAYILLLMVSQLRAVSFPEDDEPLNTVDYHCKSSRQYPVFRGRPSGNESQHRLDFQLMLKIRDTLYI
AGRDQVYTVNLNEMPKTEVIWQQKLTWRSRQQDRENCAMKGKHKDECHNFIKVFVPRNDEMVFVCGTNAFNMP
CRYYRVSTLEYDGEEISCLARCPFDARQTNVALFADGKLYSATVADFLASDAVIYRSMGDGSALRTIKYDSKW
IKEPEFLHAIEYGNYVYFFFREIAVEHNNLGKAVYSRVARICKNDMGGSQRVLEKHWTSFLKARLNCSVPGDS
FFYFDVLQSITDIIQINGIPTVVGVFTTQLNSIPGSAVCAFSMDDIEKVFKGRFKEQKTPDSVWTAVPEDKVP
KPRPGCCAKHGLAEAYKTSIDFPDETLSFIKSHPLMDSAVPPIADEPWFTKTRVRYRLTAISVDHSAGPYQNY
TVIFVGSEAGMVLKVLAKTSPFSLNDSVLLEEIEAYNHAKCSAENEEDKKVISLQLDKDHHALYVAFSSCIIR
IPLSRCERYGSCKKSCIASRDPYCGWLSQGSCGRVTPGMLLLTEDFFAFHNHSAEGYEQDTEFGNTAHLGDCH
EILPTSTTPDYKIFGGPTSGVRWEVQSGSNQMVHMNVLITCVFAAFVLGAFIAGVAVYCRYRDMFVRKNRKIH
KDAESAQSCTDSSGSFAKLNGLFDSPVKEYQQNIDSPKLYSNLLTSRKELPPNGDSKSMMVDHRGQPPELAAL
PTPESTPVLHQKTLQAMKSHSEKAHGHGASRKETPQFFPSSPPPHSPLSHGHIPSAIVLPNATHDYNTSFSNS
NAHKAEKKLQNIDHPLTKSSSKRDHRRSVDSRNTLNDLLKHLNDPNSNPKAIMGDIQMAHQNLMLDPMGSMSE
VPPKVPNREASLYSPPSTLPRNSPTKRVDVPTTPGVPMTSLERQRGYHKNSSQRHSISAMPKNLNSPNGVLLS
RQPSMNRGGYMPTPTGAKVDYIQGTPVSVHLQPSLSRQSSYTSNGTLPRTGLKRTPSLKPDVPPKPSFVPQTP
SVRPLNKYTY

| | | |
|---|---|---|
| NOV16i, CG59253-07 | SEQ ID NO: 211 | 2359 bp |
| DNA Sequence | ORF Start: ATG at 46 | ORF Stop: TGA at 2350 |

TGGCATTTCTGAGCAGGGGCCACCCTGACTTCACCTTGGCCCACCATGAGGGTCTTCCTGCTTTGTGCCTACA
TACTGCTGCTCATGGTTTCCCAGTTGAGGGCAGTCAGCTTTCCTGAAGATGATGAACCCCTTAATACTGTCGA
CTATCACTGTAAGTCGTCTAGGCAATATCCGGTTTTTAGAGGACGCCCTTCAGGCAATGAATCGCAGCACAGG
CTGGACTTTCAGCTGATGTTGAAAATTCGAGACACACTTTATATTGCTGGCAGGGATCAAGTTTATACAGTAA
ACTTAAATGAAATGCCCAAAACAGAAGTAATATGCCAACAGAAACTGACATGGCGATCAAGACAACAGGATCG
AGAAAACTGTGCTATGAAAGGCAAGCATAAAGATGAATGCCACAACTTTATCAAAGTATTTGTTCCAACAAAC
GATGAGATGGTTTTTGTTTGTGGTACCAATGCATTCAATCCCATGTGTAGATACTACAGGGTAAGTACCTTAG
AATATGATGGGGAAGAAATTAGTGGCCTGGCAAGATGCCCATTTGATGCCAGACAAACCAATGTTGCCCTCTT
TGCTGATGGGAAGCTGTATTCTGCCACAGTGGCTGACTTCTTGGCCAGCGATGCCGTTATTTATCGAAGCATG
GGTGATGGATCTGCCCTTCGCACAATAAAATATGATTCCAAATGGATAAAAGAGCCACACTTTCTTCATGCCA
TAGAATATGGAAACTATGTCTATTTCTTCTTTCGAGAAATCGCTGTCGAACATAATAATTTAGGCAAGGCTGT
GTATTCCCGCGTGGCCCGCATATGTAAAAACCACATGGGTGGTTCCCAGCGGGTCCTGGAGAAACACTGGACT
TCATTTCTAAAGGCTCGGCTGAACTGTTCTGTCCCTGGAGATTCGTTTTTCTACTTTGATGTTCTGCAGTCTA
TTACAGACATAATACAAATCAATGGCATCCCCACTGTGGTCGGGGTGTTTACCACGCAGCTCAATAGCATCCC
TGGTTCTGCTGTCTGTGCATTTAGCATGGATGACATTGAAAAAGTATTCAAAGGACGGTTTAAGGAACAGAAA
ACTCCAGATTCTGTTTGGACAGCAGTTCCCGAAGACAAAGTGCCAAAGCCAAGGCCTGGCTGTTGTGCAAAAC
ACGGCCTTGCCGAAGCTTATAAAACCTCCATCGATTTCCCGGATGAAACTCTGTCATTCATCAAATCTCATCC
CCTGATGGACTCTGCCGTTCCACCCATTGCCGATGAGCCCTGGTTCACAAAGACTCGGGTCAGGTACAGACTC
ACGGCCATCTCAGTGGACCATTCAGCCGGACCCTACCAGAACTACACAGTCATCTTTGTTGGCTCTGAAGCTG
GCATGGTACTTAAAGTTCTGGCAAAGACCAGTCCTTTCTCTTTGAACGACAGCGTATTACTGGAAGAGATTGA
AGCCTACAACCATGCAAAGTGCAGTCCTGAGAATGAGGAAGACAAAAAGGTCATCTCATTACAGTTGGATAAA
GATCACCACGCTTTATATGTGCCGTTCTCTAGCTGCATTATCCGCATCCCCCTCAGTCCCTCTGAGCGTTATG
GATCATGTAAAAAGTCTTGTATTGCATCTCGTGACCCGTATTGTGGCTGGTTAAGCCAGGGATCCTGTGGTAG
AGTGACCCCAGGGATGCTGCTGTTAACCGAAGACTTCTTTGCTTTCCATAACCACAGTGCTGAAGGATATGAA
CAAGACACAGAATTCGGCAACACAGCTCATCTAGGGGACTGCCATGAAATTTTGCCTACTTCAACTACACCAG
ATTACAAAATATTTGGCGGTCCAACATCTGACATGGAGGTATCTTCATCTTCTGTTACCACAATGGCAAGTAT
CCCAGAAATCACACCTAAAGTGATTGATACCTGGACACCTAAACTGACAAGCTCTCGGAAATTTGTAGTTCAA
GATGATCCAAACACTTCTGATTTTACTGATCCTTTATCGCGTATCCCAAAGGGTGTACGATGGGAAGTCCAGT
CTGGAGAGTCCAACCAGATGGTCCACATGAATGTCCTCATCACCTGTGTCTTTGCTGCTTTTGTTTTGGGGGC
ATTCATTGCAGGTGTGGCAGTATACTGCTATCGAGACATGTTTGTTCGGAAAAACAGAAAGATCCATAAAGAT
GCAGAGTCCGCCCAGTCATGCACAGACTCCAGTGGAAGTTTTGCCAAACTGAATGGTCTCTTTGACAGCCCTG
TCAAGGAATACCAACAGAATATTGATTCTCCTAAACTGTATAGTAACCTGCTAACCAGTCGGAAAGAGCACGA
ATTCAGCGGCCGCTGAATTCTAG

| | | | |
|---|---|---|---|
| NOV16i, CG59253-07 | | | |
| Protein Sequence | SEQ ID NO: 212 | 1768 aa | MW at 86672.6kD |

MRVFLLCAYILLLMVSQLRAVSFPEDDEPLNTVDYHCKSSRQYPVFRGRPSGNESQHRLDFQLMLKIRDTLYI
AGRDQVYTVNLNEMPKTEVIWQQKLTWRSRQQDRENCAMKGKHKDECHNFIKVFVPRNDEMVFVCGTNAFNPM
CRYYRVSTLEYDGEEISGLARCPFDARQTNVALFADGKLYSATVADFLASDAVIYRSMGDGSALRTIKYDSKW
IKEPHFLHAIEYGNYVYFFFREIAVEHNNLGKAVYSRVARICKNDMGGSQRVLEKHWTSFLKARLNCSVPGDS
FFYFDVLQSITDIIQINGIPTVVGVFTTQLNSIPGSAVCAFSMDDIEKVFKGRFKEQKTPDSVWTAVPEDKVP
KPRPGCCAKHGLAEAYKTSIDFPDETLSFIKSHPLMDSAVPPIADEPWFTKTRVRYRLTAISVDHSAGPYQNY
TVIFVGSEAGMVLKVLAKTSPFSLNDSVLLEEIEAYNHAKCSAENEEDKKVISLQLDKDHHALYVAFSSCIIR
IPLSRCERYGSCKKSCIASRDPYCGWLSQGSCGRVTPGMLLLTEDFFAFHNHSAEGYEQDTEFGNTAHLGDCH
EILPTSTTPDYKIFGGPTSDMEVSSSSVTTMASIPEITPKVIDTWRPKLTSSRKFVVQDDPNTSDFTDPLSGI
PKGVRWEVQSGESNQMVHMNVLITCVFAAFVLGAFIAGVAVYCYRDMFVRKNRKIHKDAESAQSCTDSSGSFA
KLNGLFDSPVKEYQQNIDSPKLYSNLLTSRKEHEFSGR

| | | |
|---|---|---|
| NOV16j, CG59253-08 | SEQ ID NO: 213 | 3364 bp |
| DNA Sequence | ORF Start: ATG at 46 | ORF Stop: TAG at 3310 |

TGGCATTTCTGAGCAGGGGCCACCCTGACTTCACCTTGGCCCACCATGAGGGTCTTCCTGCTTTGTGCCTACA
TACTGCTGCTGATGGTTTCCCAGTTGAGGGCAGTCAGCTTTCCTGAAGATGATGAACCCCTTAATACTGTCGA
CTATCACTGTAAGTCGTCTAGGCAATATCCGGTTTTTAGAGGACGCCCTTCAGGCAATCAATCGCAGCACAGG
CTGGACTTTCAGCTGATGTTGAAAATTCGAGACACACTTTATATTGCTGGCAGGGATCAAGTTTATACAGTAA
ACTTAAATGAAATGCCCAAAACAGAAGTAATATGGCAACAGAAACTGACATGGCCATCAAGACAACAGGATCG
AGAAAACTGTGCTATGAAAGGCAAGCATAAAGATGAATGCCACAACTTTATCAAAGTATTTGTTCCAAGAAAC
GATGAGATGGTTTTTGTTTGTGGTACCAATGCATTCAATCCCATGTGTAGATACTACAGGGTAAGTACCTTAG
AATATGATGGGGAAGAAATTAGTGGCCTGGCAAGATGCCCATTTGATGCCAGACAAACCAATGTTGCCCTCTT
TGCTGATGGGAAGCTGTATTCTGCCACAGTGGCTGACTTCTTGGCCAGCGATGCCGTTATTTATCGAAGCATG
GGTGATGGATCTCCCCTTCGCACAATAAAATATGATTCCAAATGGATAAAAGAGCCACACTTTCTTCATGCCA
TAGAATATGGAAACTATGTCTATTTCTTCTTTCGAGAAATCGCTGTCGAACATAATAATTTAGGCAAGGCTGT
GTATTCCCGCGTGGCCCGCATATGTAAAAACGACATGGGTGGTTCCCAGCGGGTCCTCGAGAAACACTGGACT
TCATTTCTAAAGGCTCGGCTGAACTGTTCTGTCCCTGGAGATTCGTTTTTCTACTTTGATGTTCTGCAGTCTA
TTACAGACATAATACAAATCAATGGCATCCCCACTGTGGTCGGGGTGTTTACCACGCAGCTCAATAGCATCCC
TGGTTCTGCTGTCTGTGCATTTAGCATGGATGACATTGAAAAAGTATTCAAAGGACGGTTTAAGGAACAGAAA
ACTCCAGATTCTGTTTGGACAGCAGTTCCCGAAGACAAAGTGCCAAAGCCAAGGCCTGGCTGTTGTGCAAAAC
ACGGCCTTGCCGAAGCTTATAAAACCTCCATCGATTTCCCGGATGAAACTCTGTCATTCATCAAATCTCATCC
CCTGATGGACTCTGCCGTTCCACCCATTGCCGATGAGCCCTGGTTCACAAAGACTCGGGTCAGGTACAGACTG
ACGGCCATCTCAGTGGACCATTCAGCCGGACCCTACCAGAACTACACAGTCATCTTTGTTGGCTCTGAAGCTG
GCATGGTACTTAAAGTTCTGGCAAAGACCAGTCCTTTCTCTTTGAACGACAGCGTATTACTGGAAGAGATTGA
AGCCTACAACCATGCAAAGTGCAGTGCTGAGAATGAGGAAGACAAAAAGGTCATCTCATTACAGTTGGATAAA
GATCACCACGCTTTATATGTGGCGTTCTCTAGCTGCATTATCCGCATCCCCCTCAGTCGCTGTGAGCGTTATG
GATCATGTAAAAAGTCTTGTATTGCATCTCGTGACCCGTATTGTGGCTGGTTAAGCCAGGGATCCTGTGGTAG
AGTCACCCCAGGGATGCTGCTGTTAACCGAAGACTTCTTTGCTTTCCATAACCACAGTGCTCAACGATATGAA
CAAGACACAGAATTCGGCAACACAGCTCATCTAGGGGACTGCCATGAAATTTTGCCTACTTCAACTACACCAG
ATTACAAAATATTTGGCGGTCCAACATCTGACATGGAGGTATCTTCATCTTCTGTTACCACAATGGCAAGTAT
CCCAGAAATCACACCTAAAGTGATTGATACCTGGACACCTAAACTGACAAGCTCTCGGAAATTTGTAGTTCAA
GATGATCCAAACACTTCTGATTTTACTGATCCTTTATCGGGTATCCCAAAGGGTGTACGATGGGAAGTCCAGT
CTGGAGAGTCCAACCAGATGGTCCACATGAATGTCCTCATCACCTGTGTCTTTGCTGCTTTTGTTTTGGGGGC
ATTCATTGCAGGTGTGGCAGTATACTGCTATCGAGACATGTTTGTTCGGAAAAACAGAAAGATCCATAAAGAT
GCAGAGTCCGCCCAGTCATGCACAGACTCCAGTGGAAGTTTTGCCAAACTGAATGGTCTCTTTCACAGCCCTG
TCAAGGAATACCAACAGAATATTGATTCTCCTAAACTGTATAGTAACCTGCTAACCAGTCGGAAAGAGCTACC
ACCCAATGGAGATACTAAATCCATGGTAATGGACCATCGAGGGCAACCTCCAGAGTTGGCTGCTCTTCCTACT
CCTGAGTCTACACCCGTGCTTCACCAGAAGACCCTGCAGGCCATCAAGAGCCACTCAGAAAAGGCCCATGGCC
ATGGAGCTTCAAGGAAAGAAACCCCTCAGTTTTTTCCGTCTAGTCCGCCACCTCATTCCCCATTAAGTCATGG
GCATATCCCCAGTGCCATTGTTCTTCCAAATGCTACCCATGACTACAACACGTCTTTCTCAAACTCCAATGCT
CACAAAGCTGAAAAGAAGCTTCAAAACATTGATCACCCTCTCACAAAGTCATCCAGTAAGAGAGATCACCGGC
GTTCTGTTGATTCCAGAAATACCCTCAATGATCTCCTGAAGCATCTGAATGACCCAAATAGTAACCCCAAAGC
CATCATGGGAGACATCCAGATGGCACACCAGAACTTAATGCTGGATCCCATGGGATCGATGTCTGAGGTCCCA
CCTAAAGTCCCTAACCGGGAGGCATCGCTATACTCCCCTCCTTCAACTCTCCCCAGAAATACCCCAACCAAGC
GAGTCCATGTCCCCACCACTCCTGGAGTCCCAATGACTTCTCTGGAAAGACAAAGAGGTTATCACAAAAATTC
CTCCCAGAGGCACTCTATATCTGCTATGCCTAAAAACTTAAACTCACCAAATCCTGTTTTGTTATCCAGACAG
CCTAGTATGAACCGTGGAGGATATATGCCCACCCCCACTGGGGCGAAGGTGGACTATATTCAGGGAACACCAG
TGAGTGTTCATCTGCAGCCTTCCCTCTCCAGACAGAGCAGCTACACCAGTAATGGCACTCTTCCTAGGACGGG
ACTAAAGAGGACGCCGTCCTTAAAACCTGACGTGCCACCAAAGCCTTCCTTTGTTCCTCAAACCCCATCTGTC
AGACCACTGAACAAATACACATACTAGGCCTCAAGTCTGCTATTCCCATGTGGCTTTATCCTGTCCGTGTTGT
TGAGAG

| | | | |
|---|---|---|---|
| NOV16j, CG59253-08 | | | |
| Protein Sequence | SEQ ID NO: 214 | 1088 aa | MW at 121674.9kD |

MRVFLLCAYILLLMVSQLRAVSFPEDDEPLNTVDYHCKSSRQYPVFRGRPSGNESQHRLDFQLMLKIRDTLYI
AGRDQVYTVNLNEMPKTEVIWQQKLTWRSRQQDRENCAMKGKHKDECHNFIKVFVPRNDEMVFVCGTNAFNPM
CRYYRVSTLEYDGEEISGLARCPFDARQTNVALFADGKLYSATVADFLASDAVIYRSMGDGSALRTIKYDSKW
IKEPHFLHAIEYGNYVYFFFREIAVEHNNLGKAVYSRVARICKNDMGGSQRVLEKHWTSFLKARLNCSVPGDS
FFYFDVLQSITDIIQINCIPTVVGVFTTQLNSIPGSAVCAFSMDDIEKVFKGRFKEQKTPDSVWTAVPEDKVP
KPRPGCCAKHGLAEAYKTSIDFPDETLSFIKSHPLMDSAVPPIADEPWFTKTRVRYRLTAISVDHSAGPYQNY
TVIFVGSEAGMVLKVLAKTSPFSLNDSVLLEEIEAYNHAKCSAENEEDKKVISLQLDKDHHALYVAFSSCIIR
IPLSRCERYGSCKKSCIASRDPYCGWLSQCSCGRVTPGMLLLTEDFFAFHNHSAEGYEQDTEFGNTAHLGDCH
EILPTSTTPDYKIFGGPTSDMEVSSSSVTTMASIPEITPKVIDTWRPKLTSSRKFVVQDDPNTSDFTDPLSGI
PKGVRWEVQSGESNQMVHMNVLITCVFAAFVLGAFIAGVAVYCYRDMFVRKNRKIHKDAESAQSCTDSSGSFA
KLNGLFDSPVKEYQQNIDSPKLYSNLLTSRKELPPNGDTKSMVMDHRGQPPELAALPTPESTPVLHQKTLQAM
KSHSEKAHGHGASRKETPQFFPSSPPPHSPLSHGHIPSAIVLPNATHDYNTSFSNSNAHKAEKKLQNIDHPLT
KSSSKRDHRRSVDSRNTLNDLLKHLNDPNSNPKAIMGDIQMAHQNLMLDPMGSMSEVPPKVPNREASLYSPPS
TLPRNSPTKRVDVPTTPGVPMTSLERQRGYHKNSSQRHSISAMPKNLNSPNGVLLSRQPSMNRGGYMPTPTGA
KVDYIQGTPVSVHLQPSLSRQSSYTSNGTLPRTGLKRTPSLKPDVPPKPSFVPQTPSVRPLNKYTY

| | | |
|---|---|---|
| NOV16k, CG59253-09 | SEQ ID NO: 215 | 3231 bp |
| DNA Sequence | ORF Start: ATG at 10 | ORF Stop: TGA at 3229 |

CGCAGATCTATGAGGGTCTTCCTGCTTTGTGCCTACATACTGCTGCTGATGGTTTCCCAGTTGAGGGCAGTCA
GCTTTCCTGAAGATGATGAACCCCTTAATACTGTCGACTATCACTATTCAACGCAATATCCGGTTTTTAGAGG
ACGCCCTTCAGGCAATGAATCGCAGCACAGGCTGGACTTTCAGCTGATGTTGAAAATTCGAGACACACTTTAT
ATTGCTGGCAGGGATCAAGTTTATACAGTAAACTTAAATGAAATGCCCAAAACAGAAGTAATACCAAACAAGA
AACTGACATGGCGATCAAGACAACAGGATCGAGAAAACTGTGCTATGAAAGGCAAGCATAAAGATGAATGCCA
CAACTTTATCAAAGTATTTGTTCCAAGAAACGATGAGATGGTTTTTGTTTGTGGTACCAATGCATTCAATCCC
ATGTGTAGATACTACAGGTTGAGTACCTTAGAATATGATGGGGAAGAAATTAGTGGCCTGGCAAGATGCCCAT
TTGATGCCAGACAAACCAATGTTGCCCTCTTTCCTGATGGGAAGCTGTATTCTGCCACAGTGGCTGACTTCTT
GGCCAGCGATGCCGTTATTTATCGAAGCATGGGTGATGGATCTGCCCTTCGCACAATAAAATATGATTCCAAA
TGGATAAAAGAGCCACACTTTCTTCATGCCATAGAATATGGAAACTATGTCTATTTCTTCTTTCGAGAAATCG
CTGTCCAACATAATAATTTAGGCAAGGCTGTGTATTCCCGCGTGGCCCGCATATGTAAAAACGACATGGGTGG
TTCCCAGCGGGTCCTGGACAAACACTGGACTTCATTTCTAAAGGCTCGGCTGAACTGTTCTGTCCCTGGAGAT
TCGTTTTTCTACTTTGATGTTCTGCAGTCTATTACAGACATAATACAAATCAATGGCATCCCCACTGTGGTCG
GGGTGTTTACCACGCAGCTCAATAGCATCCCTGGTTCTCCTGTCTGTGCATTTAGCATGGATGACATTGAAAA
AGTATTCAAAGGACGGTTTAAGGAACAGAAAACTCCAGATTCTGTTTGGACAGCAGTTCCCGAAGACAAAGTG
CCAAAGCCAAGGCCTGGCTGTTGTGCAAAACACGGCCTTGCCGAAGCTTATAAAACCTCCATCGATTTCCCGG
ATGAAACTCTGTCATTCATCAAATCTCATCCCCTGATGGACTCTGCCGTTCCACCCATTGCTGATGAGCCCTG
GTTCACAAAGACTCGGGTCAGGTACAGACTGACGCCCATCTCAGTGGACCATTCAGCCGGACCCTACCAGAAC
TACACACTCATCTTTGTTGGCTCTGAAGCTGGCATGGTACTTAAAGTTCTGCCAAAGACCAGTCCTTTCTCTT
TGAACGACAGCGTATTACTGGAAGAGATTGAAGCCTACAACCATGCAAAGTGCAGTGCTGAGAATGAGGAAGA
CAAAAAGGTCATCTCATTACAGTTGGATAAACATCACCACGCTTTATATGTGGCGTTCTCTAGCTGCATTATC
CGCATCCCCCTCAGTCGCTGTGAGCGTTATGGATCATGTAAAAAGTCTTGTATTGCATCTCGTGACCCCTATT
GTGGCTGGTTAAGCCAGGGATCCTGTGGTAGAGTGACCCCAGGGATGCTTGCTGAAGGATATGAACAAGACAC
AGAATTCGGCAACACACCTCATCTAGGGGACTGCCATGAAATTTTGCCTACTTCAACTACACCAGATTACAAA
ATATTTGGCGGTCCAACATCTGACATGGAGGTATCTTCATCTTCTGTTACCACAATGGCAAGTATCCCAGAAA
TCACACCTAAAGTGATTGATACCTGGAGACCTAAACTGACAAGCTCTCGGAAATTTGTAGTTCAAGATGATCC
AAACACTTCTCATTTTACTGATCCTTTATCGGGTATCCCAAAGGGTGTACGATGCGAAGTCCAGTCTGGAGAG
TCCAACCAGATCGTCCACATGAATGTCCTCATCACCTGTGTCTTTGCTGCTTTTGTTTTGGGGGCATTCATTG
CAGGTGTGGCAGTATACTGCTATCGAGACATGTTTGTTCGGAAAAACAGAAAGATCCATAAAGATCCAGAGTC
CGCCCAGTCATGCACAGACTCCAGTGGAAGTTTTGCCAAACTGAATGGTCTCTTTGACAGCCCTGTCAAGGAA
TACCAACAGAATATTGATTCTCCTAAACTGTATAGTAACCTGCTAACCAGTCGCAAAGAGCTACCACCCAATG
GAGATACTAAATCCATCCTAATGGACCATCGAGGGCAACCTCCAGAGTTGGCTGCTCTTCCTACTCCTGAGTC
TACACCCGTGCTTCACCAGAAGACCCTGCAGGCCATGAAGAGCCACTCAGAAAAGGCCCATGGCCATGGAGCT
TCAAGGAAAGAAACCCCTCAGTTTTTTCCGTCTAGTCCGCCACCTCATTCCCCATTAAGTCATGGGCATATCC
CCAGTGCCATTGTTCTTCCAAATGCTACCCATGACTACAACACGTCTTTCTCAAACTCCAATGCTCACAAAGC
TGAAAAGAAGCTTCAAAACATTGATCACCCTCTCACAAAGTCATCCAGTAAGAGAGATCACCGGCGTTCTGTT
GATTCCAGAAATACCCTCAATGATCTCCTGAAGCATCTGAATGACCCAAATAGTAACCCCAAAGCCATCATGG
GAGACATCCAGATGGCACACCACAACTTAATGCTGGATCCCATGGGATCGATGTCTGAGGTCCCACCTAAAGT
CCCTAACCGGGAGGCATCGCTATACTCCCCTCCTTCAACTCTCCCCAGAAATAGCCCAACCAAGCGAGTGGAT
GTCCCCACCACTCCTGCAGTCCCAATGACTTCTCTGGAAAGACAAAGAGGTTATCACAAAAATTCCTCCCAGA
GGCACTCTATATCTCCTATGCCTAAAAACTTAAACTCACCAAATGGTGTTTTGTTATCCAGACACCCTAGTAT
GAACCGTGGAGGATATATGCCCACCCCCACTGGGGCGAAGGTGGACTATATTCAGGGAACACCAGTGAGTGTT
CATCTGCAGCCTTCCCTCTCCACACACACCAGCTACACCAGTAATGGCACTCTTCCTAGCACGGGACTAAAGA
GGACGCCGTCCTTAAAACCTGACGTGCCACCAAAGCCTTCCTTTGTTCCTCAAACCCCATCTGTCAGACCACT
GAACAAATACACATACTGA

| | | | |
|---|---|---|---|
| NOV16k, CG59253-09 | | | |
| Protein Sequence | SEQ ID NO: 216 | 1073 aa | MW at 119870.9kD |

MRVFLLCAYILLLMVSQLRAVSFPEDDEPLNTVDYHYSRQYPVFRGRPSGNESQHRLDFQLMLKIRDTLYIAG
RDQVYTVNLNEMPKTEVIPNKKLTWRSRQQDRENCAMKGKHKDECHNFIKVFVPRNDEMVFVCCTNAFNPMCR
YYRLSTLEYDGEEISGLARCPFDARQTNVALFADGKLYSATVADFLASDAVIYRSMGDGSALRTIKYDSKWIK
EPHFLHAIEYGNYVYFFFREIAVEHNNLGKAVYSRVARICKNDMGGSQRVLEKHWTSFLKARLNCSVPGDSFF
YFDVLQSITDIIQINGIPTVVGVFTTQLNSIPGSAVCAFSMDDIEKVFKGRFKEQKTPDSVWTAVPEDKVPKP
RPGCCAKHGLAEAYKTSIDFPDETLSFIKSHPLMDSAVPPIADEPWFTKTRVRYRLTAISVDHSACPYQNYTV
IFVGSEAGMVLKVLAKTSPFSLNDSVLLEEIEAYNHAKCSAENEEDKKVISLQLDKDHHALYVAFSSCITRIP
LSRCERYGSCKKSCIASRDPYCGWLSQGSCGRVTPGMLAEGYEQDTEFGNTAHLGDCHEILPTSTTPDYKIFG
GPTSDMEVSSSSVTTMASIPEITPKVIDTWRPKLTSSRKFVVQDDPNTSDFTDPLSGIPKGVRWEVQSGESNQ
MVHMNVLITCVFAAFVLGAFIAGVAVYCYRDMFVRKNRKIHKDAESAQSCTDSSGSFAKLNGLFDSPVKEYQQ
NIDSPKLYSNLLTSRKELPPNGDTKSMVMDHRGQPPELAALPTPESTPVLHQKTLQAMKSHSEKAHGHGASRK
ETPQFFPSSPPPHSPLSHGHIPSAIVLPNATHDYNTSFSNSNAHKAEKKLQNIDHPLTKSSSKRDHRRSVDSR
NTLNDLLKHLNDPNSNPKAIMGDIQMAHQNLMLDPMGSMSEVPPKVPMREASLYSPPSTLPRNSPTKRVDVPT
TPGVPMTSLERQRGYHKNSSQRHSISAMPKNLNSPNGVLLSRQPSMNRGGYMPTPTGAKVDYIQGTPVSVHLQ
PSLSRQSSYTSNGTLPRTGLKRTPSLKPDVPPKPSFVPQTPSVRPLNKYTY

| | | |
|---|---|---|
| NOV16l, CG59253-10 | SEQ ID NO: 217 | 1950 bp |
| DNA Sequence | ORF Start: at 10 | ORF Stop: at 1942 |

CGCAGATCTGTCAGCTTTCCTGAAGATGATGAACCCCTTAATACTGTCGACTATCACTATTCAAGGCAATATC
CGGTTTTTAGAGGACGCCCTTCAGCCAATGAATCGCAGCACAGGCTGGACTTTCAGCTGATGTTGAAAATTCG
AGACACACTTTATATTGCTGGCAGGGATCAAGTTTATACAGTAAACTTAAATGAAATCCCCAAAACAGAACTA
ATACCCAACAAGAAACTGACATGGCGATCAAGACAACAGGATCGAGAAAACTGTGCTATGAAAGGCAAGCATA
AAGATGAATGCCACAACTTTATCAAAGTATTTGTTCCAAGAAACGATGAGATGGTTTTTGTTTGTGGTACCAA
TGCATTCAATCCCATGTGTAGATACTACAGGTTGAGTACCTTAGAATATGATGGGGAAGAAATTAGTGGCCTG
GCAAGATGCCCATTTGATGCCAGACAAACCAATCTTGCCCTCTTTGCTGATGGGAAGCTGTATTCTGCCACAG
TGGCTGACTTCTTGGCCAGCGATGCCGTTATTTATCGAAGCATGGGTGATGGATCTGCCCTTCGCACAATAAA
ATATGATTCCAAATGGATAAAAGAGCCACACTTTCTTCATGCCATAGAATATGGAAACTATGTCTATTTCTTC
TTTCGAGAAATCGCTGTCGAACATAATAATTTAGGCAAGCCTGTGTATTCCCGCGTGGCCCGCATATGTAAAA
ACGACATGGGTGGTTCCCAGCGGGTCCTGGAGAAACACTGGACTTCATTTCTAAAGGCTCGGCTGAACTGTTC
TGTCCCTGGAGATTCGTTTTTCTACTTTGATGTTCTGCAGTCTATTACAGACATAATACAAATCAATGGCATC
CCCACTGTGGTCGGGGTGTTTACCACGCAGCTCAATAGCATCCCTGGTTCTGCTGTCTGTGCATTTAGCATGG
ATGACATTGAAAAAGTATTCAAACGACGGTTTAAGGAACAGAAAACTCCAGATTCTGTTTGGACAGCAGTTCC
CGAAGACAAAGTGCCAAAGCCAAGGCCTGGCTGTTGTGCAAAACACGGCCTTGCCGAAGCTTATAAAACCTCC
ATCGATTTCCCGGATGAAACTCTGTCATTCATCAAATCTCATCCCCTGATGGACTCTCCCGTTCCACCCATTG
CTGATGAGCCCTGGTTCACAAAGACTCGGGTCAGGTACAGACTGACGGCCATCTCAGTGGACCATTCAGCCGG
ACCCTACCAGAACTACACAGTCATCTTTGTTGGCTCTGAAGCTGCCATGGTACTTAAAGTTCTGGCAAAGACC
AGTCCTTTCTCTTTGAACGACAGCGTATTACTGGAAGAGATTGAAGCCTACAACCATGCAAAGTGCAGTGCTG
AGAATGAGGAAGACAAAAAGGTCATCTCATTACAGTTGGATAAAGATCACCACGCTTTATATGTGGCGTTCTC
TAGCTGCATTATCCGCATCCCCCTCAGTCGCTGTGAGCGTTATGGATCATCTAAAAAGTCTTGTATTGCATCT
CGTGACCCGTATTGTGGCTGGTTAAGCCAGGGATCCTGTGGTAGAGTGACCCCAGGGATGCTGCTGTTAACCG
AAGACTTCTTTGCTTTCCATAACCACAGTGCTGAAGGATATGAACAAGACACAGAATTCGGCAACACACCTCA
TCTAGGGGACTGCCATGAAATTTTGCCTACTTCAACTACACCAGATTACAAAATATTTGGCGGTCCAACATCT
GACATGGAGGTATCTTCATCTTCTGTTACCACAATGGCAAGTATCCCAGAAATCACACCTAAAGTGATTGATA
CCTGGAGACCTAAACTGACAAGCTCTCGGAAATTTGTAGTTCAAGATGATCCAAACACTTCTGATTTTACTGA
TCCTTTATCGGGTATCCCAAAGGGTGTACGATGGGAAGTCCAGCTCGAGGCG

| | | | |
|---|---|---|---|
| NOV16l, CG59253-10 | | | |
| Protein Sequence | SEQ ID NO: 218 | 644 aa | MW at 72707.5kD |

VSFPEDDEPLNTVDYHYSRQYPVFRGRPSGNESQHRLDFQLMLKIRDTLYIAGRDQVYTVNLNEMPKTEVIPN
KKLTWRSRQQDRENCAMKGKHKDECHNFIKVFVPRNDEMVFVCGTNAFNPMCRYYRLSTLEYDGEEISGLARC
PFDARQTNVALFADGKLYSATVADFLASDAVIYRSMGDGSALRTIKYDSKWIKEPHFLHAIEYGNYVYFFFRE
IAVEHNNLGKAVYSRVARICKNDMGGSQRVLEKHWTSFLKARLNCSVPGDSFFYFDVLQSITDIIQINGIPTV
VGVFTTQLNSIPGSAVCAFSMDDIEKVFKGRFKEQKTPDSVWTAVPEDKVPKPRPGCCAKHGLAEAYKTSIDF
PDETLSFIKSHPLMDSAVPPIADEPWFTKTRVRYRLTAISVDHSAGPYQNYTVIFVGSEAGMVLKVLAKTSPF
SLNDSVLLEEIEAYNHAKCSAENEEDKKVISLQLDKDHHALYVAFSSCIIRIPLSRCERYGSCKKSCLASRDP
YCGWLSQCSCGRVTPGMLLLTEDFFAFHNHSAEGYEQDTEFGNTAHLGDCHEILPTSTTPDYKIFGGPTSDME
VSSSSVTTMASIPEITPKVIDTWRPKLTSSRKFVVQDDPNTSDFTDPLSGIPKGVRWEVQ

| | | |
|---|---|---|
| | SEQ ID NO: 219 | 1894 bp |
| NOV16m, SNP13381547 of | ORF Start: ATG at 46 | ORF Stop: TAG at 1474 |
| CG59253-01, DNA Sequence | SNP Pos: 215 | SNP Change: T to C |

TGGCATTTCTGAGCAGGGGCCACCCTGACTTCACCTTGGCCCACCATGAGGGTCTTCCTGCTTTGTGCCTACA
TACTGCTGCTGATGGTTTCCCAGTTGAGGGCAGTCAGCTTTCCTGAAGATGATGAACCCCTTAATACTGTCGA
CTATCACTATTCAAGGCAATATCCGGTTTTTAGAGGACCCCCTTCAGGCAATGAATCGCAGCACAGGCCGGAC
TTTCAGCTGATGTTGAAAATTCGAGACACACTTTATATTGCTGGCAGGGATCAAGTTTATACAGTAAACTTAA
ATGAAATGCCCAAAACAGAAGTAATACCCAACAAGAAACTGACATGGCGATCAAGACAACAGGATCGAGAAAA
CTGTGCTATGAAAGGCAAGCATAAAGATGAATGCCACAACTTTATCAAAGTATTTGTTCCAAGAAACGATGAG
ATGGTTTTTGTTTGTGGTACCAATCCATTCAATCCCATGTGTAGATACTACAGGTTGAGTACCTTAGAATATG
ATGGGGAAGAAATTAGTGGCCTGGCAAGATGCCCATTTCATGCCAGACAAACCAATGTTGCCCTCTTTGCTGA
TGGGAAGCTGTATTCTGCCACAGTGGCTGACTTCTTGGCCAGCGATGCCGTTATTTATCGAAGCATGGGTGAT
GCATCTGCCCTTCGCACAATAAAATATGATTCCAAATGGATAAAAGAGCCACACTTTCTTCATGCCATAGAAT
ATGGAAACTATGTCTATTTCTTCTTTCGAGAAATCGCTGTCGAACATAATAATTTAGGCAAGGCTGTGTATTC
CCGCGTGGCCCGCATATGTAAAAACGACATGGGTGGTTCCCACCGGGTCCTGGAGAAACACTGGACTTCATTT
CTAAAGGCTCGGCTGAACTGTTCTGTCCCTGGAGATTCGTTTTTCTACTTTGATGTTCTGCAGTCTATTACAG
ACATAATACAAATCAATGGCATCCCCACTGTGGTCGGGGTGTTTACCACGCACCTCAATAGCATCCCTGGTTC
TGCTGTCTGTGCATTTAGCATGGATGACATTGAAAAAGTATTCAAAGGACGGTTTAAGGAACAGAAAACTCCA
GATTCTGTTTCGACAGCAGTTCCCGAAGACAAAGTGCCAAAGCCAAGGCCTGGCTGTTGTCCAAAACACGGCC
TTGCCGAACCTTATAAAACCTCCATCGATTTCCCGGATGAAACTCTGTCATTCATCAAATCTCATCCCCTGAT
GGACTCTGCCGTTCCACCCATTGCCGATGAGCCCTGGTTCACAAAGACTCGGGTCAGGTACAGACTGACGGCC
ATCTCAGTGGACCATTCAGCCGGACCCTACCAGAACTACACAGTCATCTTTGTTGGCTCTGAAGCTGGCATGG
TACTTAAAGTTCTGGCAAAGACCAGTCCTTTCTCTTTGAACCACAGCGTATTACTGGAAGAGATTGAAGCCTA
CAACCATGCAAAGTAGGTATATGTTACGAGAACGCCCTTCAGCACTGCTCAAAAATTTTCGGCATGTATTTCA
TCTAGTCATGTCCTTTTGGTCCTCTAAATTAGCAGTGGTTTGGCATAATAGTGTTTTGTGTTTTTTTTCTCAT
TGAAATAAATCTTGGGTTTGTTTTTTTCCCGAGCCTGCTAGGGCGAGGGGGGTGAATGGTTGATGAGTTTAAA
AATAATGCAGCCCTTGTTTTTCACCTGTAGAATATGAGAACATTTTAACAGCACCTCTCTTATCTTGCAGATA
TATTCCAAGATGCTACATGCAGCAGACAGCTGTGAGCTTGCATACACACACACACAAATATACATGCACATAC
ATACACAGAATGTAGTACTAGTTAAGTATTTCCTTCCTATCTTTAATAAGTAAGAGAATATTTAGACCA

| | | | |
|---|---|---|---|
| NOV16m, SNP13381547 of | SEQ ID NO: 220 | | MW at 54200.4kD |
| CG59253-01, Protein Sequence | SNP Pos: 57 | 476 aa | SNP Change: Len to Pro |

MRVFLLCAYILLLMVSQLRAVSFPEDDEPLNTVDYHYSRQYPVFRGRPSGNESQHRPDFQLMLKIRDTLYIAG
RDQVYTVNLNEMPKTEVIPNKKLTWRSRQQDRENCANKGKHKDECHNFIKVFVPRNDEMVFVCGTNAFNPMCR
YYRLSTLEYDGEEISGLARCPFDARQTNVALFADGKLYSATVADFLASDAVIYRSMGDGSALRTIKYDSKWIK
EPHFLHAIEYGNYVYFFFREIAVEHNNLGKAVYSRVARICKNDMGGSQRVLEKHWTSFLKARLNCSVPGDSFF
YFDVLQSITDIIQINGIPTVVGVFTTQLNSIPGSAVCAFSMDDIEKVFKGRFKEQKTPDSVWTAVPEDKVPKP
RPGCCAKHGLAEAYKTSIDFPDETLSFIKSHPLMDSAVPPIADEPWFTKTRVRYRLTAISVDHSAGPYQNYTV
IFVGSEAGMVLKVLAKTSPFSLNDSVLLEEIEAYNHAK

| | | |
|---|---|---|
| | SEQ ID NO: 221 | 1894 bp |
| NOV16n, SNP13378936 of | ORF Start: ATG at 46 | ORF Stop: TAG at 1474 |
| CG59253-01, DNA Sequence | SNP Pos: 865 | SNP Change: T to C |

TGGCATTTCTGAGCAGGGGCCACCCTGACTTCACCTTGGCCCACCATGAGGGTCTTCCTGCTTTGTGCCTACA
TACTGCTCCTGATGGTTTCCCAGTTGAGGGCAGTCAGCTTTCCTGAAGATGATGAACCCCTTAATACTGTCGA
CTATCACTATTCAAGGCAATATCCGGTTTTTAGAGGACGCCCTTCAGGCAATGAATCGCAGCACAGGCTGGAC
TTTCAGCTGATGTTGAAAATTCGAGACACACTTTATATTGCTGGCAGGGATCAAGTTTATACAGTAAACTTAA
ATGAAATGCCCAAAACAGAAGTAATACCCAACAAGAAACTGACATGGCGATCAAGACAACAGGATCGAGAAAA
CTGTGCTATGAAAGGCAAGCATAAAGATGAATGCCACAACTTTATCAAAGTATTTGTTCCAAGAAACGATGAG
ATGGTTTTTGTTTGTGGTACCAATGCATTCAATCCCATGTGTAGATACTACAGGTTGAGTACCTTAGAATATG
ATGGGGAAGAAATTAGTGGCCTGGCAAGATGCCCATTTGATGCCAGACAAACCAATGTTGCCCTCTTTGCTGA
TGGGAAGCTGTATTCTGCCACAGTGGCTGACTTCTTGGCCAGCCATGCCGTTATTTATCGAAGCATGGGTGAT
GGATCTGCCCTTCGCACAATAAAATATGATTCCAAATGGATAAAAGAGCCACACTTTCTTCATGCCATAGAAT
ATGGAAACTATGTCTATTTCTTCTTTCGAGAAATCGCTGTCGAACATAATAATTTAGGCAAGGCTGTGTATTC
CCGCGTGGCCCGCATATGTAAAAACGACATGGGTGGTTCCCAGCCGGTCCTGGAGAAACACCGGACTTCATTT
CTAAAGGCTCGGCTGAACTGTTCTGTCCCTGGAGATTCGTTTTTCTACTTTGATGTTCTGCAGTCTATTACAG
ACATAATACAAATCAATGGCATCCCCACTGTGGTCGGGGTGTTTACCACGCAGCTCAATAGCATCCCTGGTTC
TGCTGTCTGTGCATTTAGCATGGATGACATTGAAAAAGTATTCAAAGGACGGTTTAAGGAACAGAAAACTCCA
GATTCTGTTTGGACAGCAGTTCCCGAAGACAAAGTGCCAAAGCCAAGGCCTGGCTGTTGTGCAAAACACGGCC
TTGCCGAAGCTTATAAAACCTCCATCGATTTCCCGGATGAAACTCTGTCATTCATCAAATCTCATCCCCTGAT
GGACTCTCCCGTTCCACCCATTGCCGATCAGCCCTGGTTCACAAAGACTCGGGTCAGGTACAGACTGACGGCC
ATCTCAGTGCACCATTCAGCCCGACCCTACCAGAACTACACAGTCATCTTTGTTGGCTCTGAAGCTGGCATGG
TACTTAAAGTTCTGGCAAAGACCAGTCCTTTCTCTTTGAACGACAGCGTATTACTGGAAGAGATTGAAGCCTA
CAACCATGCAAAGTAGGTATATGTTACGAGAACGCCCTTCAGCACTGCTCAAAAATTTTCGGCATGTATTTCA
TCTAGTCATGTCCTTTTGGTCCTCTAAATTAGCAGTGGTTTGGCATAATAGTGTTTTGTGTTTTTTTTCTCAT
TGAAATAAATCTTGGGTTTGTTTTTTTCCCGAGCCTGCTAGGGCGAGGGGGGTGAATGGTTGATGAGTTTAAA
AATAATGCAGCCCTTGTTTTTCACCTGTAGAATATGAGAACATTTTAACAGCACCTCTCTTATCTTGCAGATA
TATTCCAAGATGCTACATGCAGCAGACAGCTGTGAGCTTGCATACACACACACACAAATATACATGCACATAC
ATACACAGAATGTAGTACTAGTTAAGTATTTCCTTCCTATCTTTAATAAGTAAGAGAATATTTAGACCA

| | | | |
|---|---|---|---|
| NOV16n, SNP13378936 of | SEQ ID NO: 222 | | MW at 541 86.4kD |
| CG59253-01, Protein Sequence | SNP Pos: 274 | 476 aa | SNP Change: Trp to Arg |

MRVFLLCAYILLLMVSQLRAVSFPEDDEPLNTVDYHYSRQYPVFRGRPSGNESQHRLDFQLMLKIRDTLYIAG
RDQVYTVNLNEMPKTEVIPNKKLTWRSRQQDRENCAMKGKHKDECHNFIKVFVPRNDEMVFVCGTNAFNPMCR
YYRLSTLEYDGEEISGLARCPFDARQTNVALFADGKLYSATVADFLASDAVIYRSMGDGSALRTIKYDSKWIK
EPHFLHAIEYGNYVYFFFREIAVEHNNLGKAVYSRVARICKNDMGGSQRVLEKHRTSFLKARLNCSVPGDSFF
YFDVLQSITDIIQINGIPTVVGVFTTQLNSIPGSAVCAFSMDDIEKVFKGRFKEQKTPDSVWTAVPEDKVPKP
RPGCCAKHGLAEAYKTSIDFPDETLSFIKSHPLMDSAVPPIADEPWFTKTRVRYRLTAISVDHSACPYQNYTV
IFVGSEAGMVLKVLAKTSPFSLNDSVLLEEIEAYNHAK

| | | |
|---|---|---|
| | SEQ ID NO: 223 | 1894 bp |
| NOV16o, SNP13378935 of | ORF Start: ATG at 46 | ORF Stop: TAG at 1474 |
| CG59253-01, DNA Sequence | SNP Pos: 965 | SNP Change: A to G |

TGGCATTTCTGAGCAGGGGCCACCCTGACTTCACCTTGGCCCACCATGAGGGTCTTCCTGCTTTGTGCCTACA
TACTGCTGCTGATGGTTTCCCAGTTGAGGGCAGTCAGCTTTCCTGAAGATGATGAACCCCTTAATACTGTCGA
CTATCACTATTCAAGGCAATATCCGGTTTTTAGAGGACGCCCTTCAGGCAATGAATCGCAGCACAGGCTGGAC
TTTCAGCTGATGTTGAAAATTCGAGACACACTTTATATTGCTGGCAGGGATCAAGTTTATACAGTAAACTTAA
ATGAAATGCCCAAAACAGAAGTAATACCCAACAAGAAACTGACATGGCGATCAAGACAACAGGATCGAGAAAA
CTGTGCTATGAAAGGCAAGCATAAAGATGAATGCCACAACTTTATCAAAGTATTTGTTCCAAGAAACGATGAG
ATGGTTTTTGTTTGTGGTACCAATGCATTCAATCCCATGTGTAGATACTACAGGTTGAGTACCTTAGAATATG
ATGGGGAAGAAATTAGTGGCCTGGCAAGATGCCCATTTGATGCCAGACAAACCAATGTTGCCCTCTTTGCTGA
TGGGAAGCTGTATTCTGCCACAGTGGCTGACTTCTTGGCCAGCGATGCCGTTATTTATCGAAGCATGGGTGAT
GGATCTGCCCTTCGCACAATAAAATATGATTCCAAATGGATAAAAGAGCCACACTTTCTTCATGCCATAGAAT
ATGGAAACTATGTCTATTTCTTCTTTCGAGAAATCGCTGTCGAACATAATAATTTAGGCAAGGCTGTGTATTC
CCGCGTGGCCCGCATATGTAAAAACGACATGGGTGGTTCCCAGCGGGTCCTGGAGAAACACTGGACTTCATTT
CTAAAGGCTCGGCTGAACTGTTCTGTCCCTGGAGATTCGTTTTTCTACTTTGATGTTCTGCAGTCTATTACAG
ACATAATACAAATCAGTGGCATCCCCACTGTGGTCGGGGTGTTTACCACGCAGCTCAATAGCATCCCTGGTTC
TGCTGTCTGTGCATTTAGCATGGATGACATTGAAAAAGTATTCAAAGGACGGTTTAAGGAACACAAAACTCCA
GATTCTGTTTGGACAGCAGTTCCCGAAGACAAAGTGCCAAAGCCAAGGCCTGGCTGTTGTGCAAAACACGGCC
TTGCCGAAGCTTATAAAACCTCCATCGATTTCCCGGATGAAACTCTGTCATTCATCAAATCTCATCCCCTGAT
GGACTCTGCCGTTCCACCCATTGCCGATGAGCCCTGGTTCACAAAGACTCGGGTCAGGTACAGACTGACGGCC
ATCTCAGTGGACCATTCAGCCGGACCCTACCAGAACTACACAGTCATCTTTGTTGGCTCTGAAGCTGGCATGG
TACTTAAAGTTCTGGCAAAGACCAGTCCTTTCTCTTTGAACGACAGCGTATTACTGGAAGAGATTGAAGCCTA
CAACCATGCAAAGTAGGTATATGTTACGAGAACGCCCTTCAGCACTGCTCAAAAATTTTCGGCATGTATTTCA
TCTACTCATGTCCTTTTGGTCCTCTAAATTAGCAGTGGTTTGGCATAATAGTGTTTTGTGTTTTTTTTCTCAT
TGAAATAAATCTTGGGTTTGTTTTTTTCCCGAGCCTGCTAGGGCGAGGGGGGTGAATGGTTGATGAGTTTAAA
AATAATGCAGCCCTTGTTTTTCACCTGTAGAATATGAGAACATTTTAACAGCACCTCTCTTATCTTGCAGATA
TATTCCAAGATGCTACATGCAGCAGACAGCTGTGAGCTTGCATACACACACACACAAATATACATGCACATAC
ATACACAGAATGTAGTACTAGTTAAGTATTTCCTTCCTATCTTTAATAAGTAAGAGAATATTTAGACCA

| | | | |
|---|---|---|---|
| NOV16o, SNP13378935 of | SEQ ID NO: 224 | | MW at 54189.4kD |
| CG59253-01, Protein Sequence | SNP Pos: 307 | 476 aa | SNP Change: Asn to Ser |

MRVFLLCAYILLLMVSQLRAVSFPEDDEPLNTVDYHYSRQYPVFRGRPSGNESQHRLDFQLMLKIRDTLYIAG
RDQVYTVNLNEMPKTEVIPNKKLTWRSRQQDRENCAMKGKHKDECHNFIKVFVPRNDEMVFVCGTNAFNPMCR
YYRLSTLEYDGEEISGLARCPFDARQTNVALFADGKLYSATVADFLASDAVIYRSMGDGSALRTIKYDSKWIK
EPHFLHAIEYGNYVYFFFREIAVEHNNLGKAVYSRVARICKNDMGGSQRVLEKHWTSFLKARLINCSVPGDSFF
YFDVLQSITDIIQISGIPTVVGVFTTQLNSIPGSAVCAFSMDDIEKVFKGRFKEQKTPDSVWTAVPEDKVPKP
RPGCCAKHGLAEAYKTSIDFPDETLSFIKSHPLMDSAVPPIADEPWFTKTRVRYRLTAISVDHSAGPYQNYTV
IFVGSEAGMVLKVLAKTSPFSLNDSVLLEEIEAYNHAK

| | | |
|---|---|---|
| | SEQ ID NO: 225 | 1894 bp |
| NOV16p, SNP13381569 of | ORF Start: ATG at 46 | ORF Stop: TAG at 1474 |
| CG59253-01, DNA Sequence | SNP Pos: 1351 | SNP Change: T to C |

TGGCATTTCTGAGCAGGGGCCACCCTGACTTCACCTTGGCCCACCATGAGGGTCTTCCTGCTTTGTGCCTACA
TACTGCTGCTGATGGTTTCCCAGTTGAGGGCAGTCAGCTTTCCTGAAGATGATGAACCCCTTAATACTGTCGA
CTATCACTATTCAAGGCAATATCCGGTTTTTAGAGGACGCCCTTCAGGCAATGAATCGCAGCACAGGCTGGAC
TTTCAGCTGATGTTGAAAATTCGAGACACACTTTATATTGCTGGCAGGGATCAAGTTTATACAGTAAACTTAA
ATGAAATGCCCAAAACAGAAGTAATACCCAACAAGAAACTGACATGGCGATCAAGACAACAGGATCGAGAAAA
CTGTGCTATGAAAGGCAAGCATAAAGATGAATGCCACAACTTTATCAAAGTATTTGTTCCAAGAAACGATGAG
ATGGTTTTTGTTTGTGGTACCAATGCATTCAATCCCATGTGTAGATACTACAGGTTGAGTACCTTAGAATATG
ATGGGGAAGAAATTAGTGGCCTGGCAAGATGCCCATTTGATGCCAGACAAACCAATGTTGCCCTCTTTGCTGA
TGGGAAGCTGTATTCTGCCACAGTGGCTGACTTCTTGGCCAGCGATGCCGTTATTTATCGAAGCATGGGTGAT
GGATCTGCCCTTCGCACAATAAAATATGATTCCAAATGGATAAAAGAGCCACACTTTCTTCATGCCATAGAAT
ATGGAAACTATGTCTATTTCTTCTTCGAGAAATCGCTGTCGAACATAATAATTTAGGCAAGGCTGTGTATTC
CCGCGTGGCCCGCATATGTAAAAACGACATGGGTGGTTCCCAGCGCGTCCTGGAGAAACACTGGACTTCATTT
CTAAAGGCTCGGCTGAACTGTTCTGTCCCTGGAGATTCGTTTTTCTACTTTGATGTTCTGCAGTCTATTACAG
ACATAATACAAATCAATGGCATCCCCACTGTGGTCGGCGTGTTTACCACGCAGCTCAATAGCATCCCTGGTTC
TGCTGTCTGTGCATTTAGCATGGATGACATTGAAAAAGTATTCAAAGGACGGTTTAAGGAACAGAAAACTCCA
GATTCTGTTTGGACAGCAGTTCCCGAAGACAAAGTGCCAAAGCCAAGGCCTGGCTGTTGTGCAAAACACGGCC
TTGCCGAAGCTTATAAAACCTCCATCGATTTCCCGGATGAAACTCTGTCATTCATCAAATCTCATCCCCTGAT
GGACTCTGCCGTTCCACCCATTGCCGATGAGCCCTGGTTCACAAAGACTCGGGTCAGGTACAGACTGACGGCC
ATCTCAGTGGACCATTCAGCCGGACCCTACCAGAACCACACAGTCATCTTTGTTGGCTCTGAAGCTGGCATGG
TACTTAAAGTTCTGGCAAAGACCAGTCCTTTCTCTTTGAACGACAGCGTATTACTGGAAGAGATTGAAGCCTA
CAACCATGCAAAGTAGGTATATGTTACGAGAACGCCCTTCAGCACTGCTCAAAAATTTTCGGCATGTATTTCA
TCTAGTCATGTCCTTTTGGTCCTCTAAATTAGCAGTGGTTTGGCATAATAGTGTTTTGTGTTTTTTTTCTCAT
TGAAATAAATCTTGGGTTTGTTTTTTTCCCGAGCCTGCTAGGGCGAGGGGGGTGAATGGTTGATGAGTTTAAA
AATAATGCAGCCCTTGTTTTTCACCTGTAGAATATGAGAACATTTTAACAGCACCTCTCTTATCTTGCAGATA
TATTCCAAGATGCTACATGCAGCAGACAGCTGTGAGCTTGCATACACACACACACAAATATACATGCACATAC
ATACACAGAATGTAGTACTAGTTAAGTATTTCCTTCCTATCTTTAATAAGTAAGAGAATATTTAGACCA

| | | | |
|---|---|---|---|
| NOV16p, SNP13381569 of | SEQ ID NO: 226 | | MW at 54190.4kD |
| CG59253-01, Protein Sequence | SNP Pos: 436 | 476 aa | SNP Change: Tyr to His |

MRVFLLCAYILLLMVSQLRAVSFPEDDEPLNTVDYHYSRQYPVFRGRPSGNESQHRLDFQLMLKIRDTLYIAG
RDQVYTVNLNEMPKTEVIPNKKLTWRSRQQDRENCAMKGKHKDECHNFIKVFVPRNDEMVFVCGTNAFNPMCR
YYRLSTLEYDGEEISGLARCPFDARQTNVALFADGKLYSATVADFLASDAVIYRSMGDGSALRTIKYDSKWIK
EPHFLHAIEYGNYVYFFFREIAVEHNNLGKAVYSRVARICKNDMGGSQRVLEKHWTSFLKARLNCSVPGDSFF
YFDVLQSITDIIQINGIPTVVGVFTTQLNSIPGSAVCAFSMDDIEKVFKGRFKEQKTPDSVWTAVPEDKVPKP
RPGCCAKHGLAEAYKTSIDFPDETLSFIKSHPLMDSAVPPIADEPWFTKTRVRYRLTAISVDHSAGPYQNHTV
IFVGSEAGMVLKVLAKTSPFSLNDSVLLEEIEAYNHAK

| | | |
|---|---|---|
| | SEQ ID NO: 227 | 1894 bp |
| NOV16q, SNP13382528 of | ORF Start: ATG at 46 | ORF Stop: TAG at 1474 |
| CG59253-01, DNA Sequence | SNP Pos: 1838 ISNP Change: T to C | |

TGGCATTTCTGAGCAGGGGCCACCCTGACTTCACCTTGGCCCACCATGAGGGTCTTCCTGCTTTGTGCCTACA
TACTGCTGCTGATGGTTTCCCAGTTGAGGGCAGTCAGCTTTCCTGAAGATGATGAACCCCTTAATACTGTCGA
CTATCACTATTCAAGGCAATATCCGGTTTTTAGAGGACGCCCTTCAGGCAATGAATCGCAGCACAGGCTGGAC
TTTCAGCTGATGTTGAAAATTCGAGACACACTTTATATTGCTGGCAGGGATCAAGTTTATACAGTAAACTTAA
ATGAAATGCCCAAAACAGAAGTAATACCCAACAAGAAACTGACATGGCGATCAAGACAACAGGATCGAGAAAA
CTGTGCTATGAAAGGCAAGCATAAAGATGAATGCCACAACTTTATCAAAGTATTTGTTCCAAGAAACGATGAG
ATGGTTTTTGTTTGTGGTACCAATGCATTCAATCCCATGTGTAGATACTACAGGTTGAGTACCTTAGAATATG
ATGGGGAAGAAATTAGTGGCCTGGCAAGATGCCCATTTGATGCCAGACAAACCAATGTTGCCCTCTTTGCTGA
TGGGAAGCTGTATTCTGCCACAGTGGCTGACTTCTTGGCCAGCGATGCCGTTATTTATCGAAGCATGGGTGAT
GGATCTGCCCTTCGCACAATAAAATATGATTCCAAATGGATAAAAGAGCCACACTTTCTTCATGCCATAGAAT
ATGGAAACTATGTCTATTTCTTCTTTCGAGAAATCGCTGTCGAACATAATAATTTAGGCAAGGCTGTGTATTC
CCGCGTGGCCCGCATATGTAAAAACGACATGGGTGGTTCCCAGCGGGTCCTGGAGAAACACTGGACTTCATTT
CTAAAGGCTCGGCTGAACTGTTCTGTCCCTGGAGATTCGTTTTTCTACTTTGATGTTCTGCAGTCTATTACAG
ACATAATACAAATCAATGGCATCCCCACTGTGGTCGGGGTGTTTACCACGCAGCTCAATAGCATCCCTGGTTC
TGCTGTCTGTGCATTTAGCATGGATGACATTGAAAAAGTATTCAAAGGACGGTTTAAGGAACAGAAAACTCCA
GATTCTGTTTGGACAGCAGTTCCCGAAGACAAAGTGCCAAAGCCAAGGCCTGGCTGTTGTGCAAAACACGGCC
TTGCCGAAGCTTATAAAACCTCCATCGATTTCCCGGATGAAACTCTGTCATTCATCAAATCTCATCCCCTGAT
GGACTCTGCCGTTCCACCCATTGCCGATGAGCCCTGGTTCACAAAGACTCGGGTCAGGTACAGACTGACGGCC
ATCTCAGTGGACCATTCAGCCGGACCCTACCAGAACTACACAGTCATCTTTGTTGGCTCTGAAGCTGGCATGG
TACTTAAAGTTCTGGCAAAGACCAGTCCTTTCTCTTTGAACGACAGCGTATTACTGGAAGAGATTGAAGCCTA
CAACCATGCAAAGTAGGTATATGTTACGAGAACGCCCTTCAGCACTGCTCAAAAATTTTCGGCATGTATTTCA
TCTAGTCATGTCCTTTTGGTCCTCTAAATTAGCAGTGGTTTGGCATAATAGTGTTTTGTGTTTTTTTTCTCAT
TGAAATAAATCTTGGGTTTGTTTTTTTCCCGAGCCTGCTAGGGCGAGGGGGGTGAATGGTTGATGAGTTTAAA
AATAATGCAGCCCTTGTTTTTCACCTGTAGAATATGAGAACATTTTAACAGCACCTCTCTTATCTTGCAGATA
TATTCCAAGATGCTACATGCAGCAGACAGCTGTGAGCTTGCATACACACACACACAAATATACATGCACATAC
ATACACAGAATGCAGTACTAGTTAAGTATTTCCTTCCTATCTTTAATAAGTAAGAGAATATTTAGACCA

| | | | |
|---|---|---|---|
| NOV16q, SNP13382528 of | | | MW at 54216.4kD |
| CG59253-01, Protein Sequence | SEQ ID NO: 228 | 476 aa | SNP Change: no change |

MRVFLLCAYILLLMVSQLRAVSFPEDDEPLNTVDYHYSRQYPVFRGRPSGNESQHRLDFQLMLKIRDTLYIAG
RDQVYTVNLNEMPKTEVIPNKKLTWRSRQQDRENCAMKGKHKDECHNFIKVFVPRNDEMVFVCGTNAFNPMCR
YYRLSTLEYDGEEISGLARCPFDARQTNVALFADGKLYSATVADFLASDAVIYRSMGDGSALRTIKYDSKWIK
EPHFLHAIEYGNYVYFFFREIAVEHNNLGKAVYSRVARICKNDMGGSQRVLEKHWTSFLKARLNCSVPGDSFF
YFDVLQSITDIIQINGIPTVVGVFTTQLNSIPGSAVCAFSMDDIEKVFKGRFKEQKTPDSVWTAVPEDKVPKP
RPGCCAKHGLAEAYKTSIDFPDETLSFIKSHPLMDSAVPPIADEPWFTKTRVRYRLTAISVDHSAGPYQNYTV
IFVGSEAGMVLKVLAKTSPFSLNDSVLLEEIEAYNHAK

A ClustalW comparison of the above protein sequences yields the following sequence alignment shown in Table 16B.

TABLE 16B
Comparison of the NOV16 protein sequences.

| | |
|---|---|
| NOV16a | MRVFLLCAYILLLMVSQLRAVSFPEDDEPLNTVDYHY--SRQYPVFRGRPSGNESQHRLD |
| NOV16b | ------------------GSVSFPEDDEPLNTVDYHY--SRQYPVFRGRPSGNESQHRLD |
| NOV16c | MRVFLLCAYILLLMVSQLRAVSFPEDDEPLNTVDYHCKSSRQYPVFRGRPSGNESQHRLD |
| NOV16d | ------------------GSVSFPEDDEPLNTVDYHY--SRQYPVFRGRPSGNESQHRLD |
| NOV16e | --------------------VSFPEDDEPLNTVDYHY--SRQYPVFRGRPSGNESQHRLD |
| NOV16f | --------------------VSFPEDDEPLNTVDYHY--SRQYPVFRGRPSGNESQHRLD |
| NOV16g | MRVFLLCAYILLLMVSQLRAVSFPEDDEPLNTVDYHCKSSRQYPVFRGRPSGNESQHRLD |
| NOV16h | MRVFLLCAYILLLMVSQLRAVSFPEDDEPLNTVDYHCKSSRQYPVFRGRPSGNESQHRLD |
| NOV16i | MRVFLLCAYILLLMVSQLRAVSFPEDDEPLNTVDYHCKSSRQYPVFRGRPSGNESQHRLD |
| NOV16j | MRVFLLCAYILLLMVSQLRAVSFPEDDEPLNTVDYHCKSSRQYPVFRGRPSGNESQHRLD |
| NOV16k | MRVFLLCAYILLLMVSQLRAVSFPEDDEPLNTVDYHY--SRQYPVFRGRPSGNESQHRLD |
| NOV16l | --------------------VSFPEDDEPLNTVDYHY--SRQYPVFRGRPSGNESQHRLD |
| NOV16a | FQLMLKIRDTLYIAGRDQVYTVNLNEMPKTEVIPNKKLTWRSRQQDRENCAMKGKHKDEC |
| NOV16b | FQLMLKIRDTLYIAGRDQVYTVNLNEMPKTEVIPNKKLTWRSRQQDRENCAMKGKHKDEC |
| NOV16c | FQLMLKIRDTLYIAGRDQVYTVNLNEMPKTEVIWQQKLTWRSRQQDRENCAMKGKHKDEC |
| NOV16d | FQLMLKIRDTLYIAGGDQVYTVNLNEMPKTEVIPNKKLTWRSRQQDRENCAMKGKHKDEC |
| NOV16e | FQLMLKIRDTLYIAGRDQVYTVNLNEMPKTEVIPNKKLTWRSRQQDRENCAMKGKHKDEC |
| NOV16f | FQLMLKIRDTLYIAGGDQVYTVNLNEMPKTEVIPNKKLTWRSRQQDRENCAMKGKHKDEC |
| NOV16g | FQLMLKIRDTLYIAGRDQVYTVNLNEMPKTEVIWQQKLTWRSRQQDRENCAMKGKHKDEC |
| NOV16h | FQLMLKIRDTLYIAGRDQVYTVNLNEMPKTEVIWQQKLTWRSRQQDRENCAMKGKHKDEC |
| NOV16i | FQLMLKIRDTLYIAGRDQVYTVNLNEMPKTEVIWQQKLTWRSRQQDRENCAMKGKHKDEC |
| NOV16j | FQLMLKIRDTLYIAGRDQVYTVNLNEMPKTEVIWQQKLTWRSRQQDRENCAMKGKHKDEC |
| NOV16k | FQLMLKIRDTLYIAGRDQVYTVNLNEMPKTEVIPNKKLTWRSRQQDRENCAMKGKHKDEC |
| NOV16l | FQLMLKIRDTLYIAGRDQVYTVNLNEMPKTEVIPNKKLTWRSRQQDRENCAMKGKHKDEC |
| NOV16a | HNFIKVFVPRNDEMVFVCGTNAFNPMCRYYRLSTLEYDGEEISGLARCPFDARQTNVALF |
| NOV16b | HNFIKVFVPRNDEMVFVCGTNAFNPMCRYYRLSTLEYDGEEISGLARCPFDARQTNVALF |
| NOV16c | HNFIKVFVPRNDEMVFVCGTNAFNPMCRYYRVSTLEYDGEEISGLARCPFDARQTNVALF |
| NOV16d | HNFIKVFVPRNDEMVFVCGTNAFNPMCRYYRLSTLEYDGEEISGLARCPFDARQTNVALF |
| NOV16e | HNFIKVFVPRNDEMVFVCGTNAFNPMCRYYRLSTLEYDGEEISGLARCPFDARQTNVALF |
| NOV16f | HNFIKVFVPRNDEMVFVCGTNAFNPMCRYYRLSTLEYDGEEISGLARCPFDARQTNVALF |
| NOV16g | HNFIKVFVPRNDEMVFVCGTNAFNPMCRYYRVSTLEYDGEEISGLARCPFDARQTNVALF |
| NOV16h | HNFIKVFVPRNDEMVFVCGTNAFNPMCRYYRVSTLEYDGEEISGLARCPFDARQTNVALF |
| NOV16i | HNFIKVFVPRNDEMVFVCGTNAFNPMCRYYRVSTLEYDGEEISGLARCPFDARQTNVALF |
| NOV16j | HNFIKVFVPRNDEMVFVCGTNAFNPMCRYYRVSTLEYDGEEISGLARCPFDARQTNVALF |
| NOV16k | HNFIKVFVPRNDEMVFVCGTNAFNPMCRYYRLSTLEYDGEEISGLARCPFDARQTNVALF |
| NOV16l | HNFIKVFVPRNDEMVFVCGTNAFNPMCRYYRLSTLEYDGEEISGLARCPFDARQTNVALF |
| NOV16a | ADGKLYSATVADFLASDAVIYRSMGDGSALRTIKYDSKWIKEPHFLHAIEYGNYVYFFFR |
| NOV16b | ADGKLYSATVADFLASDAVIYRSMGDGSALRTIKYDSKWIKEPHFLHAIEYGNYVYFFFR |
| NOV16c | ADGKLYSATVADFLASDAVIYRSMGDGSALRTIKYDSKWIKEPHFLHAIEYGNYVYFFFR |
| NOV16d | ADGKLYSATVADFLASDAVIYRSMGDGSALRTIKYDSKWIKEPHFLHAIEYGNYVYFFFR |
| NOV16e | ADGKLYSATVADFLASDAVIYRSMGDGSALRTIKYDSKWIKEPHFLHAIEYGNYVYFFFR |
| NOV16f | ADGKLYSATVADFLASDAVIYRSMGDGSALRTIKYDSKWIKEPHFLHAIEYGNYVYFFFR |
| NOV16g | ADGKLYSATVADFLASDAVIYRSMGDGSALRTIKYDSKWIKEPHFLHAIEYGNYVYFFFR |
| NOV16h | ADGKLYSATVADFLASDAVIYRSMGDGSALRTIKYDSKWIKEPHFLHAIEYGNYVYFFFR |
| NOV16i | ADGKLYSATVADFLASDAVIYRSMGDGSALRTIKYDSKWIKEPHFLHAIEYGNYVYFFFR |
| NOV16j | ADGKLYSATVADFLASDAVIYRSMGDGSALRTIKYDSKWIKEPHFLHAIEYGNYVYPFFR |
| NOV16k | ADGKLYSATVADFLASDAVIYRSMGDGSALRTIKYDSKWIKEPHFLHAIEYGNYVYFFFR |
| NOV16l | ADGKLYSATVADFLASDAVIYRSMGDGSALRTIKYDSKWIKEPHFLHAIEYGNYVYFFFR |
| NOV16a | EIAVEHNNLGKAVYSRVARICKNDMGGSQRVLEKHWTSFLKARLNCSVPGDSFFYFDVLQ |
| NOV16b | EIAVEHNNLGKAVYSRVARICKNDMGGSQRVLEKHWTSFLKARLNCSVPGDSFFYFDVLQ |
| NOV16c | EIAVEHNNLGKAVYSRVARICKNDMGGSQRVLEKHWTSFLKARLNCSVPGDSFFYFDVLQ |
| NOV16d | EIAVEHNNLGKAVYSRVARICKNDMGGSQRVLEKHWTSFLKARLNCSVPGDSFFYFDVLQ |
| NOV16e | EIAVEHNNLGKAVYSRVARICKNDMGGSQRVLEKHWTSFLKARLNCSVPGDSFFYFDVLQ |
| NOV16f | EIAVEHNNLGKAVYSRVARICKNDMGGSQRVLEKHWTSFLKARLNCSVPGDSFFYFDVLQ |
| NOV16g | EIAVEHNNLGKAVYSRVARICKNDMGGSQRVLEKHWTSFLKARLNCSVPGDSFFYFDVLQ |
| NOV16h | EIAVEHNNLGKAVYSRVARICKNDMGGSQRVLEKHWTSFLKARLNCSVPGDSFFYFDVLQ |
| NOV16i | EIAVEHNNLGKAVYSRVARICKNDMGGSQRVLEKHWTSFLKARLNCSVPCDSFFYFDVLQ |
| NOV16j | EIAVEHNNLGKAVYSRVARICKNDMGGSQRVLEKHWTSFLKARLNCSVPGDSFFYFDVLQ |
| NOV16k | EIAVEHNNLGKAVYSRVARICKNDMGGSQRVLEKHWTSFLKARLNCSVPGDSFFYFDVLQ |
| NOV16l | EIAVEHNNLGKAVYSRVARICKNDMGGSQRVLEKHWTSFLKARLNCSVPGDSFFYFDVLQ |
| NOV16a | SITDIIQINGIPTVVGVFTTQLNSIPGSAVCAFSMDDIEKVFKGRFKEQKTPDSVWTAVP |
| NOV16b | SITDIIQINGIPTVVGVFTTQLNSIPGSAVCAFSMDDIEKVFKGRFKEQKTPDSVWTAVP |
| NOV16c | SITDIIQINGIPTVVGVFTTQLNSIPGSAVCAFSMDDIEKVFKGRFKEQKTPDSVWTAVP |
| NOV16d | SITDIIQINGIPTVVGVFTTQLNSIPGSAVCAFSMDDIEKVFKGRFKEQKTPDSVWTAVP |
| NOV16e | SITDIIQINGIPTVVGVFTTQLNSIPGSAVCAFSMDDIEKVFKGRFKEQKTPDSVWTAVP |
| NOV16f | SITDIIQINGIPTVVGVFTTQLNSIPGSAVCAFSMDDIEKVFKGRFKEQKTPDSVWTAVP |
| NOV16g | SITDIIQINGIPTVVGVFTTQLNSIPGSAVCAFSMDDIEKVFKGRFKEQKTPDSVWTAVP |
| NOV16h | SITDIIQINGIPTVVGVFTTQLNSIPGSAVCAFSMDDIEKVFKGRFKEQKTPDSVWTAVP |
| NOV16i | SITDIIQINGIPTVVGVFTTQLNSIPGSAVCAFSMDDIEKVFKGRFKEQKTPDSVWTAVP |
| NOV16j | SITDIIQINGIPTVVGVFTTQLNSIPGSAVCAFSMDDIEKVFKGRFKEQKTPDSVWTAVP |
| NOV16k | SITDIIQINGIPTVVGVFTTQLNSIPGSAVCAFSMDDIEKVFKGRFKEQKTPDSVWTAVP |
| NOV16l | SITDIIQINGIPTVVGVFTTQLNSIPGSAVCAFSMDDIEKVFKGRFKEQKTPDSVWTAVP |
| NOV16a | EDKVPKPRPGCCAKHGLAEAYKTSIDFPDETLSFIKSNPLMDSAVPPIADEPWFTKTRVR |
| NOV16b | EDKVPKPRPGCCAKHGLAEAYKTSIDFPDETLSFIKSHPLMDSAVPPIADEPWFTKTRVR |
| NOV16c | EDKVPKPRPGCCAKHGLAEAYKTSIDFPDETLSFIKSHPLMDSAVPPIADEPWFTKTRVR |
| NOV16d | EDKVPKPRPGCCAKHGLAEAYKTSIDFPDETLSFIKSHPLMDSAVPPIADEPWFTKTRVR |
| NOV16e | EDKVPKPRPGCCAKHGLAEAYKTSIDFPDETLSFIKSHPLMDSAVPPIADEPWFTKTRVR |
| NOV16f | EDKVPKPRPGCCAKHGLAEAYKTSIDFPDETLSFIKSHPLMDSAVPPIADEPWFTKTRVR |
| NOV16g | EDKVPKPRPGCCAKHGLAEAYKTSIDFPDETLSFIKSHPLMDSAVPPIADEPWFTKTRVR |
| NOV16h | EDKVPKPRPGCCAKHGLAEAYKTSIDFPDETLSFIKSHPLMDSAVPPIADEPWFTKTRVR |
| NOV16i | EDKVPKPRPGCCAKHGLAEAYKTSIDFPDETLSFIKSHPLMDSAVPPIADEPWFTKTRVR |
| NOV16j | EDKVPKPRPGCCAKHGLAEAYKTSIDFPDETLSFIKSHPLMDSAVPPIADEPWFTKTRVR |
| NOV16k | EDKVPKPRPGCCAKHGLAEAYKTSIDFPDETLSFIKSHPLMDSAVPPIADEPWFTKTRVR |
| NOV16l | EDKVPKPRPGCCAKHGLAEAYKTSIDFPDETLSFIKSHPLMDSAVPPIADEPWFTKTRVR |
| NOV16a | YRLTAISVDHSAGPYQNYTVIFVGSEAGMVLKVLAKTSPFSLNDSVLLEEIEAYNHAK-- |
| NOV16b | YRLTAISVDHSAGPYQNYTVIFVGSEAGMVLKVLAKTSPFSLNDSVLLEEIEAYNHAKCL |
| NOV16c | YRLTAISVDHSAGPYQNYTVIFVGSEAGMVLKVLAKTSPFSLNDSVLLEEIEAYNHAKCS |
| NOV16d | YRLTAISVDHSAGPYQNYTVIFVGSEAGMVLKVLAKTSPFSLNDSVLLEEIEAYNHAKCN |
| NOV16e | YRLTAISVDHSAGPYQNYTVIFVGSEAGMVLKVLAKTSPFSLNDSVLLEEIEAYNHAK-- |
| NOV16f | YRLTAISVDHSAGPYQNYTVIFVGSEAGMVLKVLAKTSPFSLNDSVLLEEIEAYNHAKCN |
| NOV16g | YRLTAISVDHSAGPYQNYTVIFVGSEAGMVLKVLAKTSPFSLNDSVLLEEIEAYNHAKCS |
| NOV16h | YRLTAISVDHSAGPYQNYTVIFVGSEAGMVLKVLAKTSPFSLNDSVLLEEIEAYNHAKCS |
| NOV16i | YRLTAISVDHSAGPYQNYTVIFVGSEAGMVLKVLAKTSPFSLNDSVLLEEIEAYNHAKCS |
| NOV16j | YRLTAISVDHSAGPYQNYTVIFVGSEAGMVLKVLAKTSPFSLNDSVLLEEIEAYNHAKCS |
| NOV16k | YRLTAISVDHSAGPYQNYTVIFVGSEAGMVLKVLAKTSPFSLNDSVLLEEIEAYNHAKCS |
| NOV16l | YRLTAISVDHSAGPYQNYTVIFVGSEAGMVLKVLAKTSPFSLNDSVLLEEIEAYNHAKCS |
| NOV16a | ------------------------------------------------------------ |
| NOV16b | E----------------------------------------------------------- |
| NOV16c | AENEEDKKVISLQLDKDHHALYVAFSSCIIRIPLSRCERYGSCKKSCIASRDPYCGWLSQ |
| NOV16d | AENEEDKKVISLQLDKDHHALYVAFSSCIIRIPLSRCERYGSCKKSCIASRDPYCGWLSQ |
| NOV16e | ------------------------------------------------------------ |
| NOV16f | AENEEDKKVISLQLDKDHHALYVAFSSCIIRIPLSRCERYGSCKKSCIASRDPYCGWLSQ |
| NOV16g | AENEEDKKVISLQLDKDHHALYVAFSSCIIRIPLSRCERYGSCKKSCIASRDPYCGWLSQ |
| NOV16h | AENEEDKKVISLQLDKDHHALYVAFSSCIIRIPLSRCERYGSCKKSCIASRDPYCGWLSQ |
| NOV16i | AENEEDKKVISLQLDKDHHALYVAFSSCIIRIPLSRCERYGSCKKSCIASRDPYCGWLSQ |
| NOV16j | AENEEDKKVISLQLDKDHHALYVAFSSCIIRIPLSRCERYGSCKKSCIASRDPYCGWLSQ |
| NOV16k | AENEEDKKVISLQLDKDHHALYVAFSSCIIRIPLSRCERYGSCKKSCIASRDPYCGWLSQ |
| NOV16l | AENEEDKKVISLQLDKDHHALYVAFSSCIIRIPLSRCERYGSCKKSCIASRDPYCGWLSQ |
| NOV16a | ------------------------------------------------------------ |
| NOV16b | ------------------------------------------------------------ |
| NOV16c | GSCGRVTP-------------NHSAEGYEQDTEFGNTAHLGDCHAYEPYEGR-------- |
| NOV16d | GSCGRVTP-------------GMLAEGYEQDTEFGNTAHLGD------------------ |
| NOV16e | ------------------------------------------------------------ |
| NOV16f | GSCGRVTP-------------GMLAEGYEQDTEFGNTAHLGD------------------ |
| NOV16g | GSCGRVTPGMLLLTEDFFAFHNHSAEGYEQDTEFGNTAHLCDCHEILPTS---------- |
| NOV16h | GSCGRVTPGMLLLTEDFFAFHNHSAEGYEQDTEFGNTAHLGDCHEILPTS---------- |
| NOV16i | GSCGRVTPGMLLLTEDFFAFHNHSAEGYEQDTEFGNTAHLGDCHEILPTS---------- |
| NOV16j | GSCGRVTPGMLLLTEDFFAFHNHSAEGYEQDTEFGNTAHLGDCHEILPTS---------- |
| NOV16k | GSCGRVTP-------------GMLAEGYEQDTEFGNTAHLGDCHEILPTSTTPDYKIFGG |
| NOV16l | GSCGRVTPGMLLLTEDFFAFHNHSAEGYEQDTEFGNTAHLGDCHEILPTS--------- |
| NOV16a | ------------------------------------------------------------ |
| NOV16b | ------------------------------------------------------------ |
| NOV16c | --------------------------------VGSLKAICYLLLFLKSTLFTLSHVSISG |
| NOV16d | ---------------------------------------------------------CHG |
| NOV16e | ------------------------------------------------------------ |
| NOV16f | ---------------------------------------------------------CHG |
| NOV16g | ----------------------------------------------TTPDYKIFGGPTSG |
| NOV16h | ----------------------------------------------TTPDYKIFGGPTSG |
| NOV16i | ---------------------------------------------------------TTP |
| NOV16j | ---------------------------------------------------------TTP |
| NOV16k | PTSDMEVSSSSVTTMASIPEITPKVIDTWRPKLTSSRKFVVQDDPNTSDFTDPLSGIPKG |
| NOV16l | -----------------------------------------------TTP---------- |
| NOV16a | ------------------------------------------------------------ |
| NOV16b | ------------------------------------------------------------ |
| NOV16c | VRWEVQSGESNQMVHMNVLITCVFAAFVLGAFIAGVAVYCYRDMFVRKNRKIHKDAESAQ |
| NOV16d | VRWEVQSGESNQMVHMNLE----------------------------------------- |
| NOV16e | ------------------------------------------------------------ |
| NOV16f | VRWEVQSGESNQMVHMN------------------------------------------- |
| NOV16g | VRWEVQSGESNQMVHMNVLITCVFAAFVLGAFIAGVAVYCYRDMFVRKNRKIHKDAESAQ |
| NOV16h | VRWEVQSGESNQMVHMNVLITCVFAAFVLGAFIAGVAVYCYRDMFVRKNRKIHKDAESAQ |
| NOV16i | -DYKIFGGPTSDMEVSSSSVTTMAS----IPEITPKVIDTWRPKLTSSRKFVVQDDPNTS |
| NOV16j | -DYKIFGGPTSDMEVSSSSVTTMAS----IPEITPKVIDTWRPKLTSSRKFVVQDDPNTS |
| NOV16k | VRWEVQSGESNQMVHMNVLITCVFAAFVLGAFIAGVAVYCYRDMFVRKNRKIHKDAESAQ |
| NOV16l | -DYKIFGGPTSDMEVSSSSVTTNAS----IPEITPKVIDTWRPKLTSSRKFVVQDDPNTS |
| NOV16a | ------------------------------------------------------------ |
| NOV16b | ------------------------------------------------------------ |
| NOV16c | SCTDSSGSFAKLNGLFDSPVKEYQQNIDSPKLYSNLLTSRKELPPNGDTKSMVMDHRGQP |
| NOV16d | ------------------------------------------------------------ |
| NOV16e | ------------------------------------------------------------ |
| NOV16f | ------------------------------------------------------------ |
| NOV16g | SCTDSSGSFAKLNGLFDSPVKEYQQNIDSPKLYSNLLTSRKEHEFSGR------------ |
| NOV16h | SCTDSSGSFAKLNGLFDSPVKEYQQNIDSPKLYSNLLTSRKELPPNGDSKSMVMDHRGQP |
| NOV16i | DFTDP------LSGIPKGVRWEVQSGESNQMVHMNVLITCVFAAFVLGAFIAGVAVYCYR |
| NOV16j | DFTDP------LSGIPKGVRWEVQSGESNQMVHMNVLITCVFAAFVLGAFIAGVAVYCYR |
| NOV16k | SCTDSSGSFAKLNGLFDSPVKEYQQNIDSPKLYSNLLTSRKELPPNGDTKSMVMDHRGQP |
| NOV16l | DFTDP------LSGIPKGVRWEVQ------------------------------------ |
| NOV16a | ------------------------------------------------------------ |
| NOV16b | ------------------------------------------------------------ |
| NOV16c | PELAALPTPESTPVLHQKTLQAMKSHSEKAHGHGASRKETPQFFPSSPPPHSPLSHGHIP |
| NOV16d | ------------------------------------------------------------ |
| NOV16e | ------------------------------------------------------------ |
| NOV16f | ------------------------------------------------------------ |
| NOV16g | ------------------------------------------------------------ |
| NOV16h | PELAALPTPESTPVLHQKTLQAMKSHSEKAHGHGASRKETPQFFPSSPPPHSPLSHGHIP |
| NOV16i | DMFVRKNRKIHKDAESAQSCTDSSGSFAKLNGLFDSPVKEYQQNIDSPKLYSNLLTSRKE |
| NOV16j | DMFVRKNRKIHKDAESAQSCTDSSGSFAKLNGLFDSPVKEYQQNIDSPKLYSNLLTSRKE |
| NOV16k | PELAALPTPESTPVLHQKTLQAMKSHSEKAHGHGASRKETPQFFPSSPPPHSPLSHGHIP |
| NOV16l | ------------------------------------------------------------ |
| NOV16a | ------------------------------------------------------------ |
| NOV16b | ------------------------------------------------------------ |
| NOV16c | SAIVLPNATHDYNTSFSNSNAHKAEKKLQNIDHPLTKSSSKRDHRRSVDSRNTLNDLLKH |
| NOV16d | ------------------------------------------------------------ |
| NOV16e | ------------------------------------------------------------ |
| NOV16f | ------------------------------------------------------------ |
| NOV16g | ------------------------------------------------------------ |
| NOV16h | SAIVLPNATHDYNTSFSNSNAHKAEKKLQNIDHPLTKSSSKRDHRRSVDSRNTLNDLLKH |
| NOV16i | HEFSGR------------------------------------------------------ |
| NOV16j | LPPNGDTKSMVMDHRGQPPELAALPTPESTPVLHQKTLQAMKSHSEKAHGHGASRKETPQ |
| NOV16k | SAIVLPNATHDYNTSFSNSNAHKAEKKLQNIDHPLTKSSSKRDHRRSVDSRNTLNDLLKH |
| NOV16l | ------------------------------------------------------------ |
| NOV16a | ------------------------------------------------------------ |
| NOV16b | ------------------------------------------------------------ |
| NOV16c | LNDPNSNPKAIMGDIQMAHQNLMLDPNGSMSEVPPKVPNREASLYSPPSTLPRNSPTKRV |
| NOV16d | ------------------------------------------------------------ |
| NOV16e | ------------------------------------------------------------ |
| NOV16f | ------------------------------------------------------------ |
| NOV16g | ------------------------------------------------------------ |
| NOV16h | LNDPNSNPKAIMGDIQMAHQNLMLDPMGSMSEVPPKVPNREASLYSPPSTLPRNSPTKRV |
| NOV16i | ------------------------------------------------------------ |
| NOV16j | FFPSSPPPHSPLSHGHIPSAIVLPNATHDYNTSFSNSNAHKAEKKLQNIDHPLTKSSSKR |
| NOV16k | LNDPNSNPKAIMGDIQMAHQNLMLDPMGSMSEVPPKVPNREASLYSPPSTLPRNSPTKRV |
| NOV16l | ------------------------------------------------------------ |
| NOV16a | ------------------------------------------------------------ |
| NOV16b | ------------------------------------------------------------ |
| NOV16c | DVPTTPGVPMTSLERQRGYHKNSSQRHSISAMPKNLNSPNGVLLSRQPSMNRGGYMPTPT |
| NOV16d | ------------------------------------------------------------ |
| NOV16e | ------------------------------------------------------------ |
| NOV16f | ------------------------------------------------------------ |
| NOV16g | ------------------------------------------------------------ |
| NOV16h | DVPTTPGVPMTSLERQRGYHKNSSQRHSISAMPKNLNSPNGVLLSRQPSMNRGGYMPTPT |
| NOV16i | ------------------------------------------------------------ |
| NOV16j | DHRRSVDSRNTLNDLLKHLNDPNSNPKAIMGDIQMAHQNLMLDPMGSMSEVPPKVPNREA |
| NOV16k | DVPTTPGVPMTSLERQRGYHKNSSQRHSISAMPKNLNSPNGVLLSRQPSMNRGGYMPTPT |
| NOV16l | ------------------------------------------------------------ |
| NOV16a | ------------------------------------------------------------ |
| NOV16b | ------------------------------------------------------------ |
| NOV16c | GAKVDYIQGTPVSVNLQPSLSRQSSYTSNGTLPRTGLKRTPSLKPDVPPKPSFVPQTPSV |
| NOV16d | ------------------------------------------------------------ |
| NOV16e | ------------------------------------------------------------ |
| NOV16f | ------------------------------------------------------------ |
| NOV16g | ------------------------------------------------------------ |
| NOV16h | GAKVDYIQGTPVSVHLQPSLSRQSSYTSNGTLPRTGLKRTPSLKPDVPPKPSFVPQTPSV |
| NOV16i | ------------------------------------------------------------ |
| NOV16j | SLYSPPSTLPRNSPTKRVDVPTTPGVPMTSLERQRGYHKNSSQRHSISAMPKNLNSPNGV |
| NOV16k | GAKVDYIQGTPVSVHLQPSLSRQSSYTSNGTLPRTGLKRTPSLKPDVPPKPSFVPQTPSV |
| NOV16l | ------------------------------------------------------------ |
| NOV16a | ------------------------------------------------------------ |
| NOV16b | ------------------------------------------------------------ |
| NOV16c | RPLNKYTY---------------------------------------------------- |
| NOV16d | ------------------------------------------------------------ |
| NOV16e | ------------------------------------------------------------ |
| NOV16f | ------------------------------------------------------------ |
| NOV16g | ------------------------------------------------------------ |
| NOV16h | RPLNKYTY---------------------------------------------------- |
| NOV16i | ------------------------------------------------------------ |
| NOV16j | LLSRQPSNNRGGYMPTPTGAKVDYIQGTPYSVHLQPSLSRQSSYTSNGTLPRTGLKRTPS |
| NOV16k | RPLNKYTY---------------------------------------------------- |
| NOV16l | ------------------------------------------------------------ |
| NOV16a | -------------------------- |
| NOV16b | -------------------------- |
| NOV16c | -------------------------- |
| NOV16d | -------------------------- |
| NOV16e | -------------------------- |
| NOV16f | -------------------------- |
| NOV16g | -------------------------- |
| NOV16h | -------------------------- |
| NOV16i | -------------------------- |
| NOV16j | LKPDVPPKPSFVPQTPSVRPLNKYTY |
| NOV16k | -------------------------- |
| NOV16l | -------------------------- |
| NOV16a | (SEQ ID NO: 196) |
| NOV16b | (SEQ ID NO: 198) |
| NOV16c | (SEQ ID NO: 200) |
| NOV16d | (SEQ ID NO: 202) |
| NOV16e | (SEQ ID NO: 204) |
| NOV16f | (SEQ ID NO: 206) |
| NOV16g | (SEQ ID NO: 208) |
| NOV16h | (SEQ ID NO: 210) |
| NOV16i | (SEQ ID NO: 212) |
| NOV16j | (SEQ ID NO: 214) |
| NOV16k | (SEQ ID NO: 216) |
| NOV16l | (SEQ ID NO: 218) |

Further analysis of the NOV16a protein yielded the following properties shown in Table 16C.

TABLE 16C
Protein Sequence Properties NOV16a

| SignalP | Cleavage site between residues 21 and 22 | | | |
|---|---|---|---|---|
| analysis: | | | | |
| PSORT II | PSG: a new signal peptide prediction method | | | |
| analysis: | N-region: length 2; pos. chg 1; neg. chg 0 | | | |
| | H-region: length 16; peak value 9.62 | | | |
| | PSG score: 5.22 | | | |
| | GvH: von Heijne's method for signal seq. recognition | | | |
| | GvH score (threshold: −2.1): −0.82 | | | |
| | possible cleavage site: between 20 and 21 | | | |
| | >>> Seems to have a cleavable signal peptide (1 to 20) | | | |
| | ALOM: Klein et al's method for TM region allocation | | | |
| | Init position for calculation: 21 | | | |
| | Tentative number of TMS(s) for the threshold 0.5: 1 | | | |
| | Number of TMS(s) for threshold 0.5: 0 | | | |
| | PERIPHERAL | Likelihood = 1.75 (at 300) | | |
| | ALOM score: −0.32 (number of TMSs: 0) | | | |
| | MTOP: Prediction of membrane topology (Hartmann et al.) | | | |
| | Center position for calculation: 10 | | | |
| | Charge difference: −5.0 C(−3.0)-N(2.0) | | | |
| | N >= C: N-terminal side will be inside | | | |
| | MITDISC: discrimination of mitochondrial targeting seq | | | |
| | R content: | 2 | Hyd Moment(75): | 6.62 |
| | Hyd Moment(95): | 8.11 | G content: | 0 |
| | D/E content: | 1 | S/T content: | 2 |

```
Score: −2.26
Gavel: prediction of cleavage sites for mitochondrial preseq
R-2 motif at 29 LRA|VS
NUCDISC: discrimination of nuclear localization signals
pat4: none
pat7: none
bipartite: none
content of basic residues: 12.0%
NLS Score: −0.47
KDEL: ER retention motif in the C-terminus: none
ER Membrane Retention Signals:
XXRR-like motif in the N-terminus: RVFL
none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 89
COIL: Lupas's algorithm to detect coiled-coil regions
total: 0 residues
Final Results (k = 9/23):
21.7%: mitochondrial
21.7%: endoplasmic reticulum
17.4%: extracellular, including cell wall
13.0%: vacuolar
  8.7%: cytoplasmic
  8.7%: Golgi
  8.7%: nuclear
>> prediction for CG59253-01 is mit (k = 23)
```

A search of the NOV16a protein against the Geneseq database, a proprietary database that contains sequences published in patents and patent publication, yielded several homologous proteins shown in Table 16D.

TABLE 16D
Geneseq Results for NOV16a

| | | NOV16a | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| AAE23981 | Human LP221 secreted protein - | 1 . . . 476 | 476/476 (100%) | 0.0 |
| | Homo sapiens, 476 aa. | 1 . . . 476 | 476/476 (100%) | |
| | [WO200226801-A2, 04 APR. 2002] | | | |
| AAG79413 | CADHP-2, Incyte ID No: | 1 . . . 476 | 476/476 (100%) | 0.0 |
| | 7596315CD1 - Homo sapiens, 1017 | 1 . . . 476 | 476/476 (100%) | |
| | aa. [WO200259312-A2, | | | |
| | 01 AUG. 2002] | | | |
| ABG79172 | Human semaphorin-like protein #1 - | 1 . . . 476 | 476/476 (100%) | 0.0 |
| | Homo sapiens, 476 aa. | 1 . . . 476 | 476/476 (100%) | |
| | [WO200264791-A2, 22 AUG. 2002] | | | |
| AAG63213 | Amino acid sequence of a human | 1 . . . 476 | 475/476 (99%) | 0.0 |
| | semaphorin-like polypeptide - Homo | 1 . . . 476 | 475/476 (99%) | |
| | sapiens, 1086 aa. [WO200153466-A1, | | | |
| | 26 JUL. 2001] | | | |
| ABG79177 | Human semaphorin-like protein #5 - | 1 . . . 476 | 471/478 (98%) | 0.0 |
| | Homo sapiens, 1088 aa. | 1 . . . 478 | 473/478 (98%) | |
| | [WO200264791-A2, 22 AUG. 2002] | | | |

In a BLAST search of public sequence databases, the NOV16a protein was found to have homology to the proteins shown in the BLASTP data in Table 16E.

TABLE 16E
Public BLASTP Results for NOV16a

| | | NOV16a | | |
|---|---|---|---|---|
| Protein | | Residues/ | Identities/ | |
| Accession | | Match | Similarities for the | Expect |
| Number | Protein/Organism/Length | Residues | Matched Portion | Value |
| Q8NFY6 | Semaphorin 6D isoform 2 - Homo | 1 . . . 476 | 476/476 (100%) | 0.0 |
| | sapiens (Human), 998 aa. | 1 . . . 476 | 476/476 (100%) | |
| Q8NFY5 | Semaphorin 6D isoform 3 - Homo | 1 . . . 476 | 476/476 (100%) | 0.0 |
| | sapiens (Human), 1017 aa. | 1 . . . 476 | 476/476 (100%) | |
| Q8NFY3 | Semaphorin 6D isoform 1 - Homo | 1 . . . 476 | 476/476 (100%) | 0.0 |
| | sapiens (Human), 1011 aa. | 1 . . . 476 | 476/476 (100%) | |
| Q8NFY7 | Semaphorin 6D short isoform - | 1 . . . 476 | 476/476 (100%) | 0.0 |
| | Homo sapiens (Human), 476 aa. | 1 . . . 476 | 476/476 (100%) | |
| Q9P249 | Hypothetical protein KIAA1479 - | 1 . . . 476 | 476/476 (100%) | 0.0 |
| | Homo sapiens (Human), 1022 aa | 12 . . . 487 | 476/476 (100%) | |
| | (fragment). | | | |

PFam analysis predicts that the NOV16a protein contains the domains shown in the Table 16F.

TABLE 16F
Domain Analysis of NOV16a

| | | Identities/ | |
|---|---|---|---|
| Pfam | | Similarities | Expect |
| Domain | NOV16a Match Region | for the Matched Region | Value |
| Sema | 59 . . . 476 | 197/494 (40%) | 1.3e−173 |
| | | 348/494 (70%) | |

### Example 17

The NOV17 clone was analyzed, and the nucleotide and encoded polypeptide sequences are shown in Table 17A.

TABLE 17A
NOV17 Sequence Analysis

| | | |
|---|---|---|
| NOV17a, CG95430-02 | SEQ ID NO: 229 | 954 bp |

| | | |
|---|---|---|
| DNA Sequence | ORF Start: at 7 | ORF Stop: at 949 |

GGATCCCAGGACACCTGCAGGCAAGGGCACCCTGGGATCCCTGGGAACCCCGGTCACAATGGTCTGCCTGGAA
GAGATGGACGAGACGGAGCGAAGGGTGACAAAGGCGATGCAGGAGAACCAGGACGTCCTGGCAGCCCGGGGAA
GGATGGGACGAGTGGAGAGAAGGGAGAACGAGGAGCAGATGGAAAAGTTGAAGCAAAAGGCATCAAAGGTGAT
CAACGCTCAAGAGGATCCCCAGGAAAACATGGCCCCAAGGGGCTTGCAGGGCCCATGGGAGACAAAGGCCTCC
GAGGAGAGACTGGGCCTCAGGGGCAGAAGGGGAATAACGGTGACGTGGGTCCCACTGGTCCTGAGGGGCCAAG
CGGCAACATTGGGCCTTTGGGCCCAACTGGTTTACCGGGCCCCATGGGCCCTATTGGAAAGCCTGGTCCCAAG
GGAGAAGCTGGACCCACGGGGCCCCAGGGTGAGCCAGGAGTCCGCGGAATAAGAGGCTCGAAAGGAGATCGAG
GAGAGAAAGGGAAAATCGGTGAGACTCTAGTCTTGCCAAAAAGTGCTTTCACTGTGGGGCTCACGGTGCTCAG
CAAGTTTCCTTCTTCAGATCTGCCCATTAAATTTGATAAGATCCTGTATAACGAATTCAACCATTATGATACA
GCAGCGGGGAAATTCACGTGCCACATTGCTGCCGTCTATTACTTCACCTACCACATCACTGTTTTCTCCAGGA
ATGTTCAGGTGTCTTTGGTCAAAAATGGAGTAAAAATACTGCACACCAAACATGCTTACATGAGCTCTGAGGA
CCAGGCCTCTGGCGGCATTGTCCTGCAGCTGAAGCTCGGGGATGAGGTGTGGCTGCACGTGACAGGAGGAGAG
AGGTTCAATGGCTTGTTTGCTGATGAGGACGATGACACAACTTTCACAGGGTTCCTTCTGTTCACCAGCCCGC
TCGAG

NOV17a, CG95430-02

| | | | |
|---|---|---|---|
| Protein Sequence | SEQ ID NO: 230 | 314 aa | MW at 32420.0kD |

QDTCRQGHPGIPGNPGHNGLPGRDGRDCAKGDKGDAGEPGRPGSPGKDGTSGEKGERGADGKVEAKGIKGDQG
SRGSPGKHGPKGLAGPMGEKCLRGETGPQGQKGNKGDVGPTGPEGPRGNIGPLGPTGLPGPMCPIGKPGPKGE
AGPTGPQGEPGVRGIRGWKGDRGEKGKIGETLVLPKSAFTVGLTVLSKFPSSDVPIKFDKILYNEFNHYDTAA
GKFTCHIAGVYYFTYHITVFSRNVQVSLVKNGVKILHTKDAYMSSEDQASGGIVLQLKLCDEVWLQVTGGERF
NGLFADEDDDTTFTGFLLFSSP

| | | |
|---|---|---|
| NOV17b, CG95430-04 | SEQ ID NO: 231 | 1026 bp |

| | | |
|---|---|---|
| DNA Sequence | ORF Start: ATG at 16 | ORF Stop: TGA at 1015 |

TCTGTCATCTGAACCATGAGGATCTGGTGGTTTCTGCTTGCCATTGAAATCTGCACACGGAACATAAACTCAC
AGGACACCTGCAGGCAAGGGCACCCTGGCATCCCTGGGAACCCCGGTCACAATGGTCTGTCTGGAAGAGATGG
ACGAGACGGAGCGAAGGGTGACAAAGGCGATGCAGGAGAACCAGGACGTCCTGGCAGCCCGGCGAAGGATGGG
ACGAGTGGAGACAAGGGAGAACGAGCAGCAGATGGAAAAGTTGAAGCAAAAGGCATCAAAGGTGATCAAGGCT
CAAGAGGATCCCCAGGAAAACATGGCCCCAAGGGGCTTCCAGGGCCCATGGGAGAGAAGGGCCTCCGAGGAGA
GACTGGGCCTCAGGGGCAGAAGGGGAATAAGGGTGACGTGGGTCCCACTGCTCCTGAGGGGCCAAGGGGCAAC
ATTGGGCCTTTGGGCCCAACTGGTTTACCGGGCCCCATGGGCCCTATTGGAAAGCCTGGTCCCAAAGGAGAAG
CTGCACCCACGGCGCCCCAGGGTGAGCCAGGAGTCCGGGGAATAAGAGGCTGGAAAGGAGATCGAGGAGAGAA
AGGGAAAATCGGTGAGACTCTAGTCTTGCCAAAAAGTGCTTTCACTGTGGGGCTCACGGTGCTGAGCAAGTTT
CCTTCTTCAGATATGCCCATTAAATTTGATAAGATCCTGTATAACGAATTCAACCATTATGATACAGCAGCGG
GCAAATTCACGTGCCACATTGCTGGGGTCTATTACTTCACCTACCACATCACTGTTTTCTCCAGGAATGTTCA
GGTGTCTTTGGTCAAAAATGGAGTAAAAATACTGCACACCAAAGATGCTTACATGAGCTCTGAGGACCAGGCC
TCTGGCGGCATTGTCCTGCAGCTGAAGCTCGGGCATGAGGTGTGGCTGCAGGTGACAGGAGGAGAGAGGTTCA
ATGGCTTGTTTGCTGATGAGGACGATGACACAACTTTCACAGGGTTCCTTCTGTTCAGCAGCCAGTGACAGAG
GAGA

| | |
|---|---|
| NOV17b, CG95430-04 | +TL,19 |

| | | | |
|---|---|---|---|
| Protein Sequence | SEQ ID NO: 232 | 333 aa | MW at 34735.7kD |

MRIWWFLLATEICTGNINSQDTCRQGIIPGIPGNPGHNGLSGRDGRDGAKGDKGDAGEPGRPGSPGKDGTSGEK
GERGADGKVEAKGIKGDQGSRGSPGKHGPKGLAGPMGEKGLRGETGPQGQKGNKGDVGPTGPEGPRGNIGPLG
PTGLPGPMGPIGKPGPKGEAGPTGPQGEPGVRGIRGWKGDRGEKGKIGETLVLPKSAFTVGLTVLSKFPSSDM
PIKFDKILYNEFNHYDTAAGKFTCHIAGVYYFTYHITVFSRNVQVSLVKNGVKILHTKDAYMSSEDQASGGIV
LQLKLGDEVWLQVTGGERFNGLFADEDDDTTFTGFLLFSSQ

| | | |
|---|---|---|
| NOV17c, CG95430-01 | SEQ ID NO: 233 | 818 bp |

| | | |
|---|---|---|
| DNA Sequence | ORF Start: ATG at 35 | ORF Stop: TGA at 728 |

TCCCTCTTTCAGTTCAGAGTCTGTCATCTGAACCATGAGGATCTGGTGGCTTCTGCTTGCCATTGAAATCTGC
ACAGGGAACATAAACTCACAGGACACCTGCAGGCAACGGCACCCTGGAATCCCTGCGAACCCCGGTCACAATG
GTCTGCCTGGAAGAGATGGACGAGACGGAGCGAAGGGTGACAAAGGCGATGCAGGTAAGCCTGGTCCCAAAGG
AGAAGCTGGACCCACGGGGCCCCAGGGTGAGCCAGGAGTCCGGGGAATAAGAGCCTGGAAAGGAGATCGAGGA
GAGAAAGGGAAAATCGGTGAGACTCTAGTCTTGCCAAAAAGTGCTTTCACTGTGGCGCTCACGGTGCTGAGCA
AGTTTCCTTCTTCAGATATCCCCATTAAATTTGATAAGATCCTGTATAACGAATTCAACCATTATGATACAGC
AGCGGGGAAATTCACGTGCCACATTGCTGGGGTCTATTACTTCACCTACCACATCACTGTTTTCTCCAGAAAT
GTTCAGGTGTCTTTGGTCAAAAATGGAGTAAAAATACTGCACACCAAAGATGCTTACATGAGCTCTGAGGACC
AGGCCTCTGGCGGCATTGTCCTGCAGCTGAAGCTCGGGGATGAGGTGTGGCTGCAGGTGACAGGAGGAGAGAG
GTTCAATGGCTTGTTTGCTGATGAGGACGATGACACAACTTTCACAGGGTTCCTTCTGTTCAGCAGCCCGTGA
CAGAGGAGAGTTTAAAAATCCGCCACACCATCCATCAGAATCAGCTTGGGATGAACTTATTCAGATGGTTTTA
CTTTATTAATTCCTC+TZ,1/46

NOV17c, CG95430-01

| | | | |
|---|---|---|---|
| Protein Sequence | SEQ ID NO: 234 | 231 aa | MW 24946.0kD |

MRWWLLLAIEICTGNINSQDTCRQGHPGIPGNPGHNGLPGRDGRDGAKGDKGDAGKPGPKCEAGPTGPQGEP
IGVRGIRGWKGDRGEKCKIGETLVLPKSAFTVGLTVLSKFPSSDMPIKFDKILYNEFNHYDTAAGKFTCHIAGV
IYYFTYHITVFSRNVQVSLVKNGVKILHTKDAYMSSEDQASGGIVLQLKLGDEVWLQVTGGERFNGLFADEDDD
TTFTGFLLFSSP

| | | |
|---|---|---|
| NOV17d, 319194717 | SEQ ID NO: 235 | 1024 bp |

| | | |
|---|---|---|
| DNA Sequence | ORF Start: at 2 | ORF Stop: TGA at 1013 |

CACCGGATCCACCATGAGGATCTGGTGGTTTCTGCTTGCCATTGAAATCTGCACAGGGAACATAAACTCTCAG
GACACCTGCAGGCAAGGGCACCCTGGAATCCCTGGGAACCCCGGTCACAATGGTCTGCCTGGAAGAGATGGAC
GAGACGGAGCGAAGGGTGACAAAGGCGATGCAGGAGAACCACGACGTCCTGGCAGCCCGGGGAAGGATGGGAC
GAGTGCAGAGAAGGGAGAACGAGGAGCAGATGGAAAAGTTGAAGCAAAAGGCATCAAAGGTGATCAAGGCTCA
AGACGATCCCCAGGAAAACATGGCCCCAAGGGGCTTCCAGGGCCCATGGGAGAGAAAGGCCTCCGAGGAGAGA
CTGGGCCTCAGGGGCAGAAGGGGAATAAGGGTGACGTGGGTCCCACTGGTCCTGAGGGGCCAAGGGGCAACAT
TGGGCCTTTGGGCCCAACTGGTTTACCGGGCCCCATGGGCCCTATTGGAAAGCCTGGTCCCAAGGGAGAAGCT
GCACCCACGGGGCCCCACGGTGAGCCAGGAGTCCCGGGAATAAGAGGCTGCAAAGGAGATCGAGGAGAGAAAG
GGAAAATCGGTGAGACTCTAGTCTTGCCAAAAACTGCTTTCACTGTGGGGCTCACGGTGCTGAGCAAGTTTCC
TTCTTCAGATGTGCCCATTAAATTTGATAAGATCCTGTATAACGAATTCAACCATTATGATACAGCAGCGGGG
AAATTCACCTGCCACATTGCTGGGGTCTATTACTTCACCTACCACATCACTGTTTTCTCCAGGAATGTTCAGG
TGTCTTTGGTCAAAAATGGAGTAAAAATACTGCACACCAAAGATGCTTACATGAGCTCTGAGGACCAGGCCTC
TGGCGGCATTGTCCTGCAGCTGAAGCTCGGGGATGAGGTGTGGCTGCAGGTGACAGGAGGAGAGAAGTTCAAT
GGCTTGTTTGCTGATGAGCACGATGACACAACTTTCACAGGGTTCCTTCTGTTCAGCAGCCCGTGAGTCGACG
GC+TZ,1/46

NOV17d, 319194717

| | | | |
|---|---|---|---|
| Protein Sequence | SEQ ID NO: 236 | 337 aa | Mw at 35001.0kD |

TGSTMRIWWFLLAIEICTGNINSQDTCRQGHPGIPGNPGHNGLPGRDGRDGAKGDKGDAGEPGRPGSPGKDGT
SGEKGERGADGKVEAKGIKGDQGSRGSPGKHGPKGLAGPMGEKGLRGETGPQGQKGNKGDVGPTGPEGPRGNI
GPLGPTGLPGPMGPIGKPGPKGEAGPTGPQGEPGVRGIRGWKGDRGEKGKIGETLVLPKSAFTVGLTVLSKFP
SSDVPIKFDKILYNEFNHYDTAAGKFTCHIAGVYYFTYHITVFSRNVQVSLVKNGVKILHTKDAYMSSEDQAS
GGIVLQLKLGDEVWLQVTGGEKFNGLFADEDDDTTFTGFLLFSSP

| | | |
|---|---|---|
| NOV17e, CG95430-03 | SEQ ID NO: 237 | 405 bp |

| | | |
|---|---|---|
| DNA Sequence | ORF Start: at 7 | ORF Stop: at 400 |

GGATCCGCTTTCACTGTGGGGCTCACGGTGCTGAGCAAGTTTCCTTCTTCACATATGCCCATTAAATTTGATA
AGATCCTGTATAACGAATTCAACCATTATGATACAGCAGCGGGGAAATTCACGTGCCACATTGCTGGGGTCTA
TTACTTCACCTACCACATCACTGTTTTCTCCAGGAATGTTCAGGTGTCTTTGGTCAAAAATGGAGTAAAAATA
CTGCACACCAAAGATGCTTACATGAGCTCTGAGGACCAGGCCTCTGCCGGCATTGTCCTGCAGCTGAGGCTCG
GGGATGACGTGTGGCTGCAGGTGACAGGAGGAGAGAGGTTCAATGGCTTGTTTGCTGATGAGGACGATGACAC
AACTTTCACAGGGTTCCTTCTGTTCAGCAGCCCGCTCGAG

NOV17e, CG95430-03

| | | | |
|---|---|---|---|
| Protein Sequence | SEQ ID NO: 238 | 131 aa | MW at 14607.4kD |

AFTVGLTVLSKFPSSDMPIKFDKILYNEFNHYDTAAGKFTCHIAGVYYFTYHITVFSRNVQVSLVKNGVKILH
TKDAYNSSEDQASGGIVLQLKLGDEVWLQVTGGERFNGLFADEDDDTTFTGFLLFSSP

| | | |
|---|---|---|
| NOV17f, CG95430-05 | SEQ ID NO: 239 | 149bp |

| | | |
|---|---|---|
| DNA Sequence | ORF Start: ATG at 143 | ORF Stop: TGA at 1061 |

CAGTATCTGGGTCCAGCCTGCAGCCTTAGGGTCCAGGTGATGTTACCGTGTGTCTGGCCCTTCTTCACAGTGG
CCTCCTAGAAAAACAAGACCCTGACTCAAAGAACACCTCTCACTACATTCAGAGTCTGTCATCTGAACCATGA
GGATCTGGTGGCTTCTGCTTGCCATTGAAATCTGCACAGGGAACATAAACTCACAGGACACCTGCAGGCAAGG
GCACCCTGGAATCCCTGGGAACCCCGGTCACAATGGTCTGCCTGGAAGAGATGGACGAGACGGAGCGAAGGGT
GACAAGGCGATGCAGGAGAACCAGGACGTCCTGGCAGCCCGGGGAAGGATGGGACGAGTGGAGACGAGGGGAG
AACGAGGAGCAGATGGAAAAGTTGAAGCAAAAGGCATCAAAGGTGATCAAGGCTCAAGAGGATCCCCAGGAAA
ACATGGCCCCAAGGGGCTTGCAGGGCCCATGGGAGAGAAAGGCCTCCGAGGAGAGACTGGGCCTCAGGGGCAG
AAGGGGAATAAGGCTGACGTGGGTCCCACTGGTCCTGAGGGGCCAAGGGGCAACATTGGGCCTTTGGGCCCAA
CTGGTTTACCGGGCCCCATGGGCCCTATTGGAAAGCCTGGTCCCAAGGGAGAAGCTGGACCCACGGGGCCCCA
GGGTGAGCCAGGAGTCCGGGGAATAAGAGGCTGGAAAGGAGATCGAGGAGAGAAAGGGAAAATCGGTGAGACT
CTAGTCTTGCCAAAAAGTGCTTTCACTGTGGGGCTCACGGTGCTGAGCAAGTTTCCTTCTTCAGATGTGCCCA
TTAAATTTGATAAGATCCACATCACTGTTTTCTCCAGGAATGTTCAGGTGTCTTTGGTCAAAAACGGAGTAAA
AATACTGCACACCAGAGATGCTTACGTGAGCTCTGAGGACCAGGCCTCTGGCAGCATTGTCCTGCAGCTGAAG
CTCGGGGATGAGATGTGGCTGCAGGTGACAGGAGGAGAGAGGTTCAATGGCTTGTTTGCTGATGAGGACGATG
ACACAACTTTCACAGGGTTCCTTCTGTTCAGCAGCCAGTGACAGAGGAGAGTTTATAAATCTGCCAGACCATC
CATCAGAATCAGCTTGGGATGAACTTATTCAGATGGTTTTACTTTATTAATTCA+TZ,1/46

NOV17f, CG95430-05

| | | | |
|---|---|---|---|
| Protein Sequence | SEQ ID NO: 240 | 306 aa | MW at 31546.2kD |

MRIWWLLLAIEICTGNINSQDTCRQGHPGIPGNPGHNGLPGRDGRDGAKGDKGDAGEPGRPGSPGKDGTSGEK
GERGADGKVEAKGIKGDQGSRGSPGKHGPKGLAGPMGEKGLRGETGPQGQKGNKGDVGPTGPEGPRGNIGPLG
PTGLPGPMGPIGKPGPKGEAGPTGPQGEPGVRGIRGWKGDRGEKGKIGETLVLPKSAFTVGLTVLSKFPSSDV
PTKFDKIHITVFSRNVQVSLVKNGVKILHTRDAYVSSEDQASGSIVLQLKLGDEMWLQVTGGERFNGLFADED
DDTTFTGFLLFSSQ

| | | |
|---|---|---|
| NOV17g, CG95430-06 | SEQ ID NO: 241 | 889 bp |

| | | |
|---|---|---|
| DNA Sequence | ORF Start: ATG at 16 | ORF Stop: TGA at 880 |

TCTGTCATCTGAACCATGAGGATCTGGTGGTTTCTGCTTGCCATTGAAATCTGCACAGGGAACATAAACTCAC
AGGACACCTGCAGGCAAGGGCACCCTGGCATCCCTGGGAACCCCGGTCACAATGGTCTGTCTGGAAGAGATGG
ACGAGACGGAGCCAAGGGTGACAAAGGCGATGCAGGAGAACCAGGACGTCCTGGCAGCCCGGGGAAGGATGGG
ACGAGTGGAGAGAAGGGAGAACGAGGAGCAGATGGAAAAGTTGAAGCAAAAGGCATCAAAGGTGATCAAGGCT
CAAGAGGATCCCCAGGAAAACATGGCCCCAAGGGGCTTGCAGGGCCCATGGGAOAGAAACGCCTCCGAGGAGA
GACTGGGCCTCAGGCGCAGAAGGGGAATAAGGGTGAGCCAGGAGTCCGGGGAATAAGAGGCTGGAAAGGAGAT
CGAGGAGAGAAAGGGAAAATCGGTGAGACTCTAGTCTTGCCAAAAAGTGCTTTCACTGTGGGGCTCACGGTGC
TGAGCAAGTTTCCTTCTTCAGATGTGCCCATTAAATTTGATAAGATCCTGTATAACGAATTCAACCATTATGA
TACAGCAGCCGGGAAATTCACGTGCCACATTGCTGGGGTCTATTACTTCACCTACCACATCGCTGTTTTCTCC
AGCAATGTTCAGGTGTCTTTGGTCAAAAATGGAGTAAAAATACTGCACACCAAAGATGCTTACATGAGCTCTG
AGGACCAGGCCTCTGGCGGCATTGTCCTGCAGCTGAAGCTCGGGGATGAGGTGTGGCTGCAGGTGACAGGAGG
AGAGAGGTTCAATGGCTTGTTTGCTGATGAGGACGATGACACAACTTTCACAGGGTTCCTTCTGTTCAGCACC
CAGTGACAGAGGA+TZ,1/46

NOV17g, CG95430-06

| | | | |
|---|---|---|---|
| Protein Sequence | SEQ ID NO: 242 | 288 aa | Mw at 30497.9kD |

MRIWWFLLAIEICTGNINSQDTCRQGHPGIPGNPGHNGLSGRDGRDGAKGDKGDAGEPGRPGSPGKDGTSGEK
GERGADGKVEAKGTKGDQGSRGSPGKHGPKGLAGPMGEKGLRGETGPQGQKGNKGEPGVRGIRGWKGDRGEKG
KIGETLVLPKSAFTVGLTVLSKFPSSDVPIKFDKILYNEFNHYDTAAGKFTCHIAGVYYFTYHIAVFSSNVQV
SLVKNGVKILHTKDAYMSSEDQASGGIVLQLKLGDEVWLQVTGGERFNGLFADEDDDTTFTGFLLFSSQ

| | | |
|---|---|---|
| NOV17h, CG95430-07 | SEQ ID NO: 243 | 961 bp |

| | | |
|---|---|---|
| DNA Sequence | ORF Start: at 11 | ORF Stop: at 953 |

CACCAGATCTCAGGACACCTGCAGGCAAGGGCACCCTGGGATCCCTGGGAACCCCGGTCACAATGGTCTGCCT
GCAAGAGATGGACGAGACGGAGCGAAGGGTGACAAAGGCGATGCAGGAGAACCAGGACGTCCTGGCAGCCCGG
GGAAGGATGGGACGAGTGGAGAGAAGGGAGAACGAGGAGCAGATGGAAAAGTTGAAGCAAAAGGCATCAAAGG
TGATCAAGGCTCAAGAGGATCCCCAGGAAAACATGGCCCCAAGGGGCTTGCAGGGCCCATGGGAGAGAAAGGC
CTCCGAGGAGAGACTGGGCCTCAGGGGCAGAAGGGGAATAAGGGTGACGTGGGTCCCACTGGTCCTGAGGGGC
CAAGGGGCAACATTGGGCCTTTGGGCCCAACTGGTTTACCGGGCCCCATGGGCCCTATTGGAAAGCCTGGTCC
CAAGGGAGAAGCTGGACCCACGGGGCCCCAGGGTGAGCCAGGAGTCCGGGGAATAAGAGGCTGGAAAGGAGAT
CGAGGAGAGAAAGGGAAAATCGGTGAGACTCTAGTCTTGCCAAAAAGTGCTTTCACTGTGGGGCTCACGGTGC
TGACCAAGTTTCCTTCTTCAGATGTGCCCATTAAATTTGATAAGATCCTGTATAACGAATTCAACCATTATGA
TACAGCAGCGGGGAAATTCACGTCCCACATTGCTGGGGTCTATTACTTCACCTACCACATCACTGTTTTCTCC
ACGAATGTTCAGGTGTCTTTGGTCAAAAATGGAGTAAAAATACTGCACACCAAAGATGCTTACATGAGCTCTG
AGGACCAGGCCTCTGGCGGCATTGTCCTGCAGCTGAAGCTCGGGGATGAGGTGTGGCTGCAGGTGACAGGAGG
AGAGAGGTTCAATGGCTTGTTTGCTGATGAGGACGATGACACAACTTTCACAGGGTTCCTTCTGTTCAGCAGC
CCGGTCGACGGC+TZ,1/46

NOV17h, CG95430-07

| | | | |
|---|---|---|---|
| Protein Sequence | SEQ ID NO: 244 | 314 aa | Mw at 32420.0kD |

QDTCRQGHPGIPGNPGHNGLPGRDGRDGAKGDKGDAGEPGRPGSPGKDGTSGEKGERGADGKVEAKGIKGDQG
SRGSPGKHGPKGLAGPMGEKGLRGETGPQGQKGNKGDVGPTGPEGPRGNIGPLGPTGLPGPMGPIGKPGPKGE
AGPTGPQGEPGVRGIRGWKGDRGEKGKIGETLVLPKSAFTVGLTVLSKFPSSDVPIKFDKILYNEFNHYDTAA
GKFTCHIAGVYYFTYHITVFSRNVQVSLVKNGVKILHTKDAYMSSEDQASGGIVLQLKLGDEVWLQVTGGERF
NGLFADEDDDTTFTGFLLFSSP

| | | |
|---|---|---|
| NOV17i, CG95430-08 | SEQ ID NO: 245 | 1024 bp |

| | | |
|---|---|---|
| DNA Sequence | ORF Start: ATG at 14 | ORF Stop: TGA at 1013 |

CACCGGATCCACCATGAGGATCTGGTGGTTTCTGCTTGCCATTGAAATCTGCACAGGGAACATAAACTCAG
GACACCTGCAGGCAAGGGCACCCTGGAATCCCTGGCAACCCCGGTCACAATGGTCTGCCTGGAACAGATGGAC
GAGACGGAGCGAAGGGTGACAAAOGCGATGCAGGAGAACCAGGACGTCCTGGCAGCCCGGGGAAGGATGGGAC
GAGTGGAGAGAAGGGAGAACGAGGAGCAGATGGAAAAGTTGAAGCAAAAGGCATCAAAGGTGATCAAGGCTCA
AGAGGATCCCCAGGAAAACATGGCCCCAAGGGGCTTGCAGGGCCCATGGGAGAGAAAGGCCTCCGAGGAGAGA
CTGGGCCTCAGGGGCAGAAGGGGAATAAGGGTGACGTGGGTCCCACTGGTCCTGAGGGGCCAAGGGGCAACAT
TGGGCCTTTGGGCCCAACTGGTTTACCGCGCCCCATGGGCCCTATTGGAAAGCCTGGTCCCAAGGGAGAAGCT
GGACCCACGGGCCCCCAGGGTGAGCCAGGAGTCCGGGGAATAAGAGGCTGGAAAGGAGATCGAGGAGAGAAAG
GGAAAATCGGTGAGACTCTAGTCTTGCCAAAAAGTGCTTTCACTGTGGGGCTCACGGTGCTGAGCAAGTTTCC
TTCTTCAGATGTGCCCATTAAATTTGATAAGATCCTGTATAACGAATTCAACCATTATCATACAGCAGCGGGG
AAATTCACGTGCCACATTGCTGGGGTCTATTACTTCACCTACCACATCACTGTTTTCTCCAGGAATGTTCAGG
TGTCTTTGGTCAAAAATCGAGTAAAAATACTGCACACCAAAGATGCTTACATGAGCTCTGAGGACCAGGCCTC
TGGCGGCATTGTCCTGCAGCTGAAGCTCGGGGATGAGGTGTGGCTGCAGGTGACAGGAGCAGAGAAGTTCAAT
GGCTTGTTTGCTGATGAGGACGATGACACAACTTTCACAGGGTTCCTTCTGTTCAGCAGCCCGTGAGTCGACG
GC+TZ,1/46

NOV17i, CG95430-08

| | | | |
|---|---|---|---|
| Protein Sequence | SEQ ID NO: 246 | 333 aa | MW at 34654.7kD |

MRIWWFLLAIEICTGNINSQDTCRQGHPGIPGNPGHNGLPGRDGRDGAKGDKGDAGEPCRPGSPGKDGTSCEK
GERGADGKVEAKGIKGDQGSRGSPGKHGPKGLAGPMGEKGLRGETGPQGQKGNKGDVGPTGPEGPRGNIGPLG
PTGLPCPMGPIGKPGPKGEAGPTGPQGEPGVRGIRGWKGDRGEKGKIGETLVLPKSAFTVCLTVLSKFPSSDV
PIKFDKILYNEFNHYDTAAGKFTCHIAGVYYFTYHITVFSRNVQVSLVKNGVKILHTKDAYMSSEDQASGGIV
LQLKLGDEVWLQVTGGEKFNGLFADEDDDTTFTGFLLFSSP

| | | |
|---|---|---|
| NOV 17j, CG95430-09 | SEQ ID NO: 247 | 964 bp |

| | | |
|---|---|---|
| DNA Sequence | ORF Start: at 11 | ORF Stop: TAG at 953 |

CACCAGATCTCAGGACACCTCCAGGCAAGGGCACCCTGGGATCCCTGGGAACCCCGGTCACAATGGTCTGCCT
GGAAGAGATGGACGAGACGGAGCGAAGGGTGACAAAGGCGATGCAGGAGAACCAGGACGTCCTGGCAGCCCGG
CGAAGGATGGGACGAGTGGAGAGAAGGGAGAACGAGGAGCAGATGGAAAAGTTCAAGCAAAAGGCATCAAAGG
TGATCAAGGCTCAAGAGGATCCCCAGGAAAACATGGCCCCAAGGGGCTTGCAGGGCCCATGGGAGAGAAAGGC
CTCCGAGGAGAGACTGGGCCTCAGGGGCAGAAGGGGAATAAGGGTGACGTGGGTCCCACTGGTCCTGAGGGGC
CAAGGGGCAACATTGGGCCTTTGGGCCCAACTGGTTTACCGGGCCCCATGGGCCCTATTGGAAAGCCTGGTCC
CAAGGGAGAAGCTGGACCCACGGGGCCCCAGGGTGAGCCAGGAGTCCGGGGAATAAGAGGCTGCAAAGGAGAT
CGAGGAGAGAAAGGGAAAATCGGTGAGACTCTAGTCTTGCCAAAAAGTGCTTTCACTGTGGGGCTCACGGTGC
TGAGCAAGTTTCCTTCTTCAGATGTGCCCATTAAATTTGATAAGATCCTGTATAACGAATTCAACCATTATGA
TACAGCAGCGGGGAAATTCACCTGCCACATTGCTGGGGTCTATTACTTCACCTACCACATCACTGTTTTCTCC
AGGAATGTTCAGGTGTCTTTGGTCAAAAATGGAGTAAAAATACTGCACACCAAAGATGCTTACATGAGCTCTG
AGGACCAGGCCTCTGGCGGCATTGTCCTGCAGCTGAAGCTCGGGGATGAGGTGTGGCTGCAGGTGACAGGAGG
AGAGAGGTTCAATGGCTTGTTTGCTGATGAGGACGATGACACAACTTTCACAGGGTTCCTTCTGTTCAGCAGC
CCGTAGGTCGACGGC+TZ,1/46

NOV17j, CG95430-09

| | | | |
|---|---|---|---|
| Protein Sequence | SEQ ID NO: 248 | 314 aa | MW at 32420.0kD |

QDTCRQGHPGIPGNPGHNGLPGRDGRDGAKGDKGDAGEPGRPGSPGKDGTSGEKGERGADGKVEAKGIKGDQG
SRGSPGKHGPKGLAGPMGEKGLRGETGPQGQKGNKGDVGPTGPEGPRGNIGPLGPTGLPGPMGPIGKPGPKGE
AGPTGPQGEPGVRGIRGWKGDRGEKGKIGETLVLPKSAFTVGLTVLSKFPSSDVPIKFDKILYNEFNHYDTAA
GKFTCHIAGVYYFTYHITVFSRNVQVSLVKNGVKILHTKDAYMSSEDQASGGIVLQLKLGDEVWLQVTGGERF
NGLFADEDDDTTFTGFLLFSSP

| | | |
|---|---|---|
| NOV17k, CG95430-10 | SEQ ID NO:249 | 1024 bp |

| | | |
|---|---|---|
| DNA Sequence | ORF Start: ATG at 17 | ORF Stop: at 1016 |

CACCAGATCTCCCACCATGAGGATCTGGTGGTTTCTGCTTGCCATTGAAATCTGCACAGGGAACATAAACTCT
CAGCACACCTGCAGGCAACCGCACCCTGGAATCCCTGGGAACCCCGGTCACAATGGTCTGCCTGGAAGAGATG
GACGAGACGGAGCGAAGGGTGACAAAGGCGATGCAGGAGAACCAGGACGTCCTGGCAGCCCGGGGAAGGATGG
GACGAGTGGAGAGAAGGGAGAACGAGGAGCAGATGGAAAAGTTGAAGCAAAAGGCATCAAAGGTGATCAAGGC
TCAACAGGATCCCCAGGAAAACATGGCCCCAAGGGGCTTCCAGGGCCCATGGGAGAGAAAGGCCTCCGAGGAG
AGACTGGGCCTCAGGGGCAGAAGGGGAATAAGGGTGACGTGGGTCCCACTGGTCCTGAGGGGCCAAGGGGCAA
CATTGGGCCTTTCGGCCCAACTGGTTTACCGGGCCCCATGGGCCCTATTGGAAAGCCTGGTCCCAAGGGAGAA
GCTGGACCCACGGGGCCCCAGGGTGAGCCAGGAGTCCGGGGAATAAGAGGCTGGAAAGGAGATCGAGGAGAGA
AAGGGAAAATCGGTGAGACTCTAGTCTTGCCAAAAAGTGCTTTCACTGTGGGGCTCACGGTGCTGAGCAAGTT
TCCTTCTTCAGATGTGCCCATTAAATTTGATAAGATCCTGTATAACGAATTCAACCATTATGATACAGCAGCG
GGGAAATTCACGTGCCACATTGCTGGGGTCTATTACTTCACCTACCACATCACTGTTTTCTCCAGGAATGTTC
AGGTGTCTTTGGTCAAAAATGGAGTAAAAATACTGCACACCAAACATGCTTACATGAGCTCTGAGGACCAGGC
CTCTGGCGGCATTGTCCTGCAGCTGAAGCTCGGGGATGAGGTGTGGCTGCACGTGACAGGAGCAGAGAAGTTC
AATGGCTTGTTTGCTCATGAGGACGATGACACAACTTTCACAGGGTTCCTTCTGTTCAGCAGCCCGCTCGAGG
GC+TZ,1/46

NOV17k, CG95430-10

| | | | |
|---|---|---|---|
| Protein Sequence | SEQ ID NO: 250 | 333 aa | MW at 34654.7kD |

MRIWWFLLAIEICTGNIMSQDTCRQGHPGIPGMPGHNGLPGRDGRDGAKGDKGDAGEPGRPGSPGKDGTSGEK
GERGADGKVEAKGIKGDQGSRGSPGKHGPKGLAGPMGEKGLRGETCPQGQKGNKGDVGPTGPEGPRGNIGPLG
PTGLPGPMGPIGKPGPKGEAGPTGPQGEPGVRGIRGWKGDRGEKGKIGETLVLPKSAFTVGLTVLSKFPSSDV
PIKFDKILYNEFNHYDTAAGKFTCHIAGVYYFTYHITVFSRNVQVSLVKNGVKILHTKDAYMSSEDQASGGIV
LQLKLGDEVWLQVTGGEKFNGLFADEDDDTTFTGFLLFSSP

| | | |
|---|---|---|
| NOV17l, CG95430-11 | SEQ ID NO: 251 | 1045 bp |

| | | |
|---|---|---|
| DNA Sequence | ORF Start: ATG at 14 | ORF Stop: TAA at 1034 |

CACCAGATCTACCATGGGCCACCATCACCACCATCACAGGATCTGGTGGTTTCTGCTTGCCATTGAAATCTCC
ACAGGGAACATAAACTCTCACGACACCTGCAGGCAAGGGCACCCTGGAATCCCTGCGAACCCCGGTCACAATG
GTCTGCCTGGAAGAGATGGACGAGACGGAGCGAAGGGTGACAAAGGCGATGCAGGAGAACCAGGACGTCCTGG
CACCCCGGGGAAGGATGGCACGAGTGGAGAGAAGGGAGAACGAGGAGCAGATGGAAAAGTTGAAGCAAAAGGC
ATCAAAGGTGATCAAGGCTCAAGAGGATCCCCAGGAAAACATGGCCCCAACGGGCTTGCAGGGCCCATGGGAG
AGAAAGGCCTCCGAGGAGAGACTGGGCCTCAGGGGCAGAAGGGGAATAAGGGTGACGTGGGTCCCACTGGTCC
TGAGGGGCCAAGGGGCAACATTGGGCCTTTGGGCCCAACTGGTTTACCGGGCCCCATGGGCCCTATTGGAAAG
CCTGGTCCCAACGGAGAAGCTGGACCCACGGGGCCCCAGGGTGAGCCAGGAGTCCGGGGAATAAGAGGCTGGA
AAGGAGATCGAGGAGACAAAGGGAAAATCGGTGAGACTCTAGTCTTGCCAAAAAGTGCTTTCACTGTGGGGCT
CACGGTGCTGAGCAAGTTTCCTTCTTCAGATGTGCCCATTAAATTTGATAAGATCCTGTATAACGAATTCAAC
CATTATGATACAGCAGCGGGGAAATTCACGTGCCACATTGCTGGGGTCTATTACTTCACCTACCACATCACTG
TTTTCTCCAGGAATGTTCAGGTGTCTTTGGTCAAAAATGGAGTAAAAATACTGCACACCAAAGATGCTTACAT
GAGCTCTGAGGACCACGCCTCTGGCGGCATTGTCCTGCAGCTGAAGCTCCGGGATGAGGTGTGGCTGCAGGTG
ACAGGAGGAGAGAAGTTCAATGGCTTGTTTGCTGATGAGGACCATGACACAACTTTCACAGGGTTCCTTCTGT
TCAGCAGCCCGTAACTCGAGGGC+TZ,1/46

NOV17l, CG95430-11

| | | | |
|---|---|---|---|
| Protein Sequence | SEQ ID NO: 252 | 340 aa | MW at 35534.6kD |

MGHHHHHHRIWNFLLAIEICTGNINSQDTCRQGHPGIPGNPGHNGLPGRDGRDGAKGDKGDAGEPGRPGSPGK
DGTSGEKGERGADGKVEAKGLKGDQGSRGSPGKHGPKGLAGPMGEKGLRGETGPQGQKGNKGDVGPTGPEGPR
GNIGPLGPTGLPGPMGPIGKPGPKGEAGPTGPQGEPGVRGIRGWKGDRGEKGKIGETLVLPKSAFTVGLTVLS
KFPSSDVPIKFDKILYNEFNHYDTAAGKFTCHIAGVYYFTYHITVFSRNVQVSLVKNGVKILHTKDAYMSSED
QASGGIVLQLKLGDEVWLQVTGGEKFNGLFADEDDDTTFTGFLLFSSP

| | | |
|---|---|---|
| NOV17m, CG95430-12 | SEQ ID NO: 253 | 982 bp |

| | | |
|---|---|---|
| DNA Sequence | ORF Start: at 11 | ORF Stop: TAA at 971 |

CACCAGATCTCACCATCACCACCATCACCAGGACACCTGCAGGCAAGGGCACCCTGGAATCCCTCGGAACCCC
GGTCACAATGGTCTGCCTGGAAGAGATGGACGAGACGGAGCGAAGGGTGACAAAGGCGATGCAGGAGAACCAG
GACGTCCTGGCAGCCCGGGGAAGGATGGGACGAGTGGAGAGAAGGGAGAACGAGGAGCAGATGGAAAAGTTGA
AGCAAAAGGCATCAAAGGTGATCAAGGCTCAAGAGGATCCCCAGGAAAACATGGCCCCAAGCGGCTTGCAGGG
CCCATGGGAGAGAAAGGCCTCCGAGGAGACACTGGGCCTCAGGGGCAGAAGGGGAATAAGGGTGACGTGGGTC
CCACTGGTCCTGAGGGGCCAAGGGGCAACATTGGGCCTTTGGGCCCAACTCGTTTACCGGGCCCCATGGGCCC
TATTGGAAAGCCTGGTCCCAACGGAGAAGCTGGACCCACGGGCCCCCAGGGTGAGCCAGGAGTCCGGGGAATA
AGAGGCTGGAAAGGAGATCGAGGAGAGAAAGGGAAAATCGGTGAGACTCTAGTCTTGCCAAAAAGTGCTTTCA
CTGTGGGGCTCACGGTGCTGAGCAAGTTTCCTTCTTCAGATGTGCCCATTAAATTTGATAAGATCCTGTATAA
CGAATTCAACCATTATGATACAGCAGCGGGGAAATTCACGTGCCACATTGCTGGGGTCTATTACTTCACCTAC
CACATCACTGTTTTCTCCAGGAATGTTCAGGTGTCTTTGGTCAAAAATGGAGTAAPAATACTGCACACCAAAG
ATGCTTACATGAGCTCTGAGGACCAGGCCTCTGGCGGCATTGTCCTGCAGCTGAAGCTCCGGGATGACGTGTG
GCTGCAGGTGACAGGAGGAGAGAAGTTCAATGGCTTGTTTGCTGATGAGGACGATGACACAACTTTCACAGGG
TTCCTTCTGTTCAGCAGCCCGTAACTCGAGGGC+TZ,1/46

NOV17m, CG95430-12

| | | | |
|---|---|---|---|
| Protein Sequence | SEQ ID NO: 254 | 320 aa | MW at 33214.9kD |

HHHHHHQDTCRQGHPGIPGNPGHNGLPGRDGRDGAKGDKGDAGEPGRPGSPGKDGTSGEKGERGADGKVEAKG
IKGDQGSRGSPGKHGPKGLAGPMGEKGLRGETGPQGQKGNKGDVGPTGPEGPRGNICPLGPTGLPGPMGPIGK
PGPKGEAGPTGPQGEPGVRGIRGWKGDRGEKGKIGETLVLPKSAFTVGLTVLSKFPSSDVPIKFDKILYMEFN
HYDTAAGKFTCHIAGVYYFTYHITVFSRNVQVSLVKNGVKILHTKDAYMSSEDQASGGIVLQLKLGDEVWLQV
TGGEKFNGLFADEDDDTTFTGFLLFSSP

| | | |
|---|---|---|
| NOV17n, CG95430-13 | SEQ ID NO: 255 | 982 bp |

| | | |
|---|---|---|
| DNA Sequence | ORF Start: at 11 | ORF Stop: TAA at 971 |

CACCAGATCTCACCATCACCACCATCACCAGGACACCTGCAGGCAAGGGCACCCTGGAATCCCTGGGAACCCC
GGTCACAATGGTCTGCCTGGAAGAGATGGACGAGACGCAGCGAAGGGTGACAAAGGCGATGCAGGAGAACCAG
GACGTCCTGGCAGCCCGGGGAAGGATGGGACGAGTGGAGAGAAGGGAGAACGAGGAGCAGATGGAAAAGTTGA
AGCAAAAGGCATCAAAGGTGATCAAGGCTCAAGAGGATCCCCAGGAAAACATGGCCCCAAGGCGCTTGCAGGG
CCCATGGGAGAGAAAGGCCTCCGAGGAGAGACTGGGCCTCAGGGGCAGAAGGGGAATAAGGGTGACGTGGGTC
CCACTGGTCCTGAGGGGCCAAGGGGCAACATTGGGCCTTTGGGCCCAACTGGTTTACCGGGCCCCATGGGCCC
TATTGGAAAGCCTGGTCCCAAGGGAGAAGCTGGACCCACGGGGCCCCAGGGTGAGCCAGGAGTCCGGCGAATA
AGAGGCTGGAAAGCAGATCGAGGAGAGAAAGGGAAAATCGGTGAGACTCTAGTCTTGCCAAAAAGTGCTTTCA
CTGTCCGGCTCACGGTGCTGAGCAAGTTTCCTTCTTCAGATGTGCCCATTAAATTTGATAACATCCTGTATAA
CGAATTCAACCATTATGATACAGCACCGGGGAAATTCACGTGCCACATTGCTGGGGTCTATTACTTCACCTAC
CACATCACTGTTTTCTCCAGGAATGTTCAGGTGTCTTTGGTCAAAAATGGAGTAAAAATACTGCACACCAAAG
ATGCTTACATGAGCTCTCAGGACCAGGCCTCTGGCGGCATTGTCCTGCAGCTGAAGCTCGGGGATGAGGTGTG
GCTGCAGGTGACAGGAGGAGAGAGGTTCAATGGCTTGTTTGCTGATGAGGACGATGACACAACTTTCACAGGG
TTCCTTCTGTTCAGCAGCCCGTAACTCGAGGGC+TZ,1/46

NOV17n, CG95430-13

| | | | |
|---|---|---|---|
| Protein Sequence | SEQ ID NO: 256 | 320 aa | MW at 33242.9kD |

HHHHHHQDTCRQGHPGIPGNPGHNGLPGRDGRDGAKGDKGDAGEPGRPGSPCKDGTSGEKGERGADGKVEAKG
IKGDQGSRGSPGKHGPKGLAGPMGEKGLRGETGPQGQKGNKGDVGPTGPEGPRGNIGPLCPTGLPGPMGPIGK
PGPKGEAGPTGPQGEPGVRGIRGWKGDRGEKGKIGETLVLPKSAFTVGLTVLSKFPSSDVPIKFDKILYNEFN
HYDTAAGKFTCHIAGVYYFTYHITVFSRNVQVSLVKNGVKILHTKDAYMSSEDQASGGIVLQLKLGDEVWLQV
TGGERFNGLFADEDDDTTFTGFLLFSSP

| | |
|---|---|
| SEQ ID NO: 257 | 818 bp |

| | | |
|---|---|---|
| NOV17o, SNP13379412 of | ORF Start: ATG at 35 | ORF Stop: TGA at 728 |

| | | |
|---|---|---|
| CG95430-01, DNA Sequence | SNP Pos: 50 | SNP Change: C to T |

TCCCTCTTTCAGTTCAGAGTCTGTCATCTGAACCATGAGGATCTGGTGGTTTCTGCTTGCCATTGAAATCTGC
ACAGGGAACATAAACTCACAGGACACCTGCAGGCAAGCGCACCCTGGAATCCCTGGGAACCCCGGTCACAATG
GTCTGCCTGGAAGAGATGGACGAGACGGAGCGAAGGGTGACAAAGGCGATGCAGGTAAGCCTGGTCCCAAAGG
AGAAGCTCGACCCACGGGGCCCCAGGGTGAGCCAGGAGTCCGGGGAATAAGAGGCTGGAAAGGAGATCGAGGA
GAGAAAGGGAAAATCGGTGAGACTCTAGTCTTGCCAAAAAGTGCTTTCACTGTGGGGCTCACGGTGCTGAGCA
AGTTTCCTTCTTCAGATATGCCCATTAAATTTGATAAGATCCTGTATAACGAATTCAACCATTATGATACAGC
AGCGGGGAAATTCACGTGCCACATTGCTGGGGTCTATTACTTCACCTACCACATCACTGTTTTCTCCAGAAAT
GTTCAGGTGTCTTTGGTCAAAAATGGAGTAAAAATACTGCACACCAAAGATGCTTACATGAGCTCTGAGGACC
AGGCCTCTGGCGGCATTGTCCTGCAGCTGAACCTCGGGGATGAGGTGTGGCTGCAGGTGACAGGAGGAGAGAG
GTTCAATGGCTTGTTTGCTGATGAGGACGATGACACAACTTTCACAGGGTTCCTTCTGTTCAGCAGCCCGTGA
CAGAGGAGAGTTTAAAAATCCGCCACACCATCCATCAGAATCAGCTTGGGATGAACTTATTCAGATGGTTTTA
CTTTATTAATTCCTC+TZ,1/46

| | | |
|---|---|---|
| NOV17o, SNP13379412 of | SEQ ID NO: 258 | MW at 24980.0kD |

| | | | |
|---|---|---|---|
| CG95430-01, Protein Sequence | SNP Pos: 6 | 231 aa | SNP Change: Leu to Phe |

MRIWWFLLAIEICTGNINSQDTCRQGHPGIPGNPGHNGLPGRDGRDGAKGDKGDAGKPGPKGEAGPTGPQGEP
GVRGIRGWKGDRGEKGKIGETLVLPKSAFTVGLTVLSKFPSSDMPIKFDKILYNEFNHYDTAAGKFTCHIAGV
YYFTYHITVFSRNVQVSLVKNGVKILHTKDAYMSSEDQASGGIVLQLKLGDEVWLQVTGGERFNGLFADEDDD
TTFTGFLLFSSP

| | |
|---|---|
| SEQ ID NO: 259 | 818 bp |

| | | |
|---|---|---|
| NOV17p, SNP13381828 of | ORF Start: ATG at 35 | ORF Stop: TGA at 728 |

| | | |
|---|---|---|
| CG95430-01, DNA Sequence | SNP Pos: 235 | SNP Change: G to A |

TCCCTCTTTCAGTTCAGAGTCTGTCATCTGAACCATGAGGATCTGGTGGCTTCTGCTTGCCATTGAAATCTGC
ACAGGGAACATAAACTCACAGGACACCTGCAGGCAAGGGCACCCTGGAATCCCTGGGAACCCCGGTCACAATG
GTCTGCCTGGAAGAGATGGACGAGACGGAGCGAAGGCTGACAAAGGCGATGCAGGTAAGCCTGGTCCCAAAGG
AGAAGCTGGACCCACAGGGCCCCAGGGTGAGCCAGGAGTCCGGGGAATAAGAGGCTGGAAAGGAGATCGAGGA
GAGAAAGGGAAAATCGGTGACACTCTAGTCTTGCCAAAAAGTGCTTTCACTGTGGGGCTCACGGTGCTGAGCA
AGTTTCCTTCTTCAGATATGCCCATTAAATTTGATAAGATCCTGTATAACGAATTCAACCATTATGATACAGC
AGCGGGCAAATTCACGTGCCACATTCCTGGGGTCTATTACTTCACCTACCACATCACTGTTTTCTCCAGAAAT
GTTCAGGTGTCTTTGGTCAAAAATGGAGTAAAAATACTGCACACCAAAGATGCTTACATGAGCTCTGAGGACC
AGCCCTCTGGCGGCATTCTCCTGCAGCTGAAGCTCGGGCATGAGGTGTGGCTGCAGGTGACAGGAGGAGAGAG
GTTCAATGGCTTGTTTGCTGATGAGGACGATGACACAACTTTCACAGGGTTCCTTCTGTTCAGCAGCCCGTGA
CAGACGAGAGTTTAAAAATCCGCCACACCATCCATCAGAATCAGCTTGGGATGAACTTATTCAGATGGTTTTA
CTTTATTAATTCCTC+TZ,1/46

| | | |
|---|---|---|
| NOV17p, SNP13381828 of | SEQ ID NO: 260 | MW at 24913.9kD |

| | | | |
|---|---|---|---|
| CG95430-01, Protein Sequence | SNP Pos: 67 | 231 aa | SNP Change: Thr to Thr |

MRIWWLLLAIEICTGNINSQDTCRQGHPGIPGNPGHNGLPGRDGRDGAKGDKGDAGKPGPKGEAGPTGPQGEP
GVRGIRGWKGDRGEKGKIGETLVLPKSAFTVGLTVLSKFPSSDVPIKFDKILYNEFNHYDTAAGKFTCHIAGV
YYFTYHITVFSRNVQVSLVKNGVKILHTKDAYMSSEDQASGGIVLQLKLGDEVWLQVTGGERFNGLFADEDDD
TTFTGFLLFSSP

| | |
|---|---|
| SEQ ID NO: 261 | 818 bp |

| | | |
|---|---|---|
| NOV17q, SNP13379125 of | ORF Start: ATG at 35 | ORF Stop: TGA at 728 |

| | | |
|---|---|---|
| CG95430-01, DNA Sequence | SNP Pos: 383 | SNP Change: A to G |

TCCCTCTTTCAGTTCAGAGTCTGTCATCTGAACCATGAGGATCTGGTGGCTTCTGCTTCCCATTGAAATCTGC
ACAGGCAACATAAACTCACAGGACACCTGCAGGCAAGGGCACCCTGGAATCCCTGGGAACCCCGGTCACAATG
GTCTGCCTGGAAGAGATGGACGAGACGGAGCGAAGGGTGACAAAGGCGATGCAGGTAAGCCTGGTCCCAAAGG
AGAAGCTGGACCCACGGGGCCCCAGGGTGAGCCAGGAGTCCGGGGAATAAGACGCTGGAAAGGAGATCGAGGA
GAGAAAGCGAAAATCGGTGAGACTCTACTCTTGCCAAAAAGTGCTTTCACTGTGGGGCTCACGGTGCTGAGCA
AGTTTCCTTCTTCAGATGTGCCCATTAAATTTGATAAGATCCTGTATAACGAATTCAACCATTATGATACAGC
ACCGGGGAAATTCACGTGCCACATTGCTGGGGTCTATTACTTCACCTACCACATCACTGTTTTCTCCAGAAAT
GTTCAGGTGTCTTTGGTCAAAAATGGAGTAAAAATACTGCACACCAAAGATGCTTACATGAGCTCTGAGGACC
AGGCCTCTGGCGGCATTGTCCTGCAGCTGAAGCTCGGGGATGAGGTGTCGCTGCAGGTGACAGGAGGAGAGAG
GTTCAATGGCTTGTTTGCTGATGAGGACGATGACACAACTTTCACAGGGTTCCTTCTGTTCAGCAGCCCGTGA
CAGAGGAGAGTTTAAAAATCCGCCACACCATCCATCAGAATCAGCTTGGGATGAACTTATTCAGATGGTTTTA
CTTTATTAATTCCTC+TZ,1/46

| | | |
|---|---|---|
| NOV17q, SNP13379125 of | SEQ ID NO: 262 | MW at 24913.9kD |

| | | | |
|---|---|---|---|
| CG95430-01, Protein Sequence | SNP Pos: 117 | 231 aa | SNP Change: Met to Val |

MRIWWLLLAIEICTGNINSQDTCRQGHPGIPGNPGHNGLPGRDGRDGAKGDKGDAGKPGPKGEAGPTGPQGEP
GVRGIRGWKGDRGEKGKIGETLVLPKSAFTVGLTVLSKFPSSDVPIKFDKILYNEFNHYDTAAGKFTCHIAGV
YYFTYHITVFSRNVQVSLVKNGVKILHTKDAYMSSEDQASGGIVLQLKLGDEVWLQVTGGERFNGLFADEDDD
TTFTGFLLFSSP

| | |
|---|---|
| SEQ ID NO: 263 | 818 bp |

| | | |
|---|---|---|
| NOV17r, SNP13381827 of | ORF Start: ATG at 35 | ORF Stop: TGA at 728 |

| | | |
|---|---|---|
| CG95430-01, DNA Sequence | SNP Pos: 650 | SNP Change: G to A |

TCCCTCTTTCAGTTCACAGTCTGTCATCTGAACCATGAGGATCTGGTGGCTTCTGCTTGCCATTGAAATCTGC
ACAGGGAACATAAACTCACAGGACACCTGCAGGCAAGGGCACCCTGGAATCCCTGGGAACCCCGGTCACAATG
GTCTGCCTGGAAGAGATGGACGAGACGGAGCGAAGGGTGACAAAGGCGATGCAGGTAAGCCTGGTCCCAAAGG
AGAAGCTGGACCCACGGGGCCCCAGGGTGAGCCAGGAGTCCGGGGAATAAGAGGCTGGAAAGGACATCGAGGA
GAGAAACCGAAAATCGGTGAGACTCTAGTCTTGCCAAAAAGTGCTTTCACTGTGGGGCTCACCGTGCTGAGCA
AGTTTCCTTCTTCAGATATGCCCATTAAATTTGATAAGATCCTGTATAACGAATTCAACCATTATGATACAGC
AGCGGGGAAATTCACGTGCCACATTGCTGGGGTCTATTACTTCACCTACCACATCACTGTTTTCTCCAGAAAT
GTTCAGGTGTCTTTGGTCAAAAATGGAGTAAAAATACTGCACACCAAAGATGCTTACATGAGCTCTGAGGACC
AGGCCTCTGGCGGCATTGTCCTGCAGCTGAAGCTCGGGGATGAGGTGTGGCTGCAGGTGACAGGAAGAGAGAG
GTTCAATGCCTTGTTTGCTGATGAGCACGATGACACAACTTTCACAGGCTTCCTTCTGTTCAGCAGCCCGTGA
CAGAGGAGAGTTTAAAAATCCGCCACACCATCCATCAGAATCAGCTTGGGATGAACTTATTCAGATGGTTTTA
CTTTATTAATTCCTC+TZ,1/46

| | | |
|---|---|---|
| NOV17r, SNP13381827 of | SEQ ID NO: 264 | MW at 25045.1kD |

| | | | |
|---|---|---|---|
| CG95430-01, Protein Sequence | SNP Pos: 206 | 231 aa | SNP Change: Gly to Arg |

MRIWWLLLAIEICTGNINSQDTCRQGHPGIPGNPGHNGLPGRDGRDGAKGDKGDAGKPGPKGEAGPTGPQGEP
GVRGIRGWKGDRGEKGKIGETLVLPKSAFTVGLTVLSKFPSSDMPIKFDKILYNEFNHYDTAAGKFTCHIAGV
YYFTYHITVFSRNVQVSLVKNGVKILHTKDAYMSSEDQASGGIVLQLKLGDEVWLQVTGRERFNGLFADEDDD
TTFTGFLLFSSP

| | |
|---|---|
| SEQ ID NO: 265 | 818 bp |

| | | |
|---|---|---|
| NOV17s, SNP13381822 of | ORF Start: ATG at 35 | ORF Stop: TGA at 728 |

| | | |
|---|---|---|
| CG95430-01, DNA Sequence | SNP Pos: 687 | SNP Change: A to G |

TCCCTCTTTCAGTTCAGAGTCTGTCATCTGAACCATGAGGATCTGGTGGCTTCTGCTTGCCATTGAAATCTGC
ACAGGGAACATAAACTCACAGGACACCTCCAGGCAAGGGCACCCTGGAATCCCTGGGAACCCCGGTCACAATG
GTCTGCCTGGAAGAGATGGACGAGACGGAGCGAAGGGTGACAAAGGCGATGCAGGTAAGCCTGGTCCCAAAGG
ACAAGCTGGACCCACGGGGCCCCAGGGTGAGCCAGGAGTCCGGGGAATAACAGGCTGGAAAGGAGATCGAGGA
GAGAAAGGGAAAATCGGTGAGACTCTAGTCTTGCCAAAAAGTGCTTTCACTGTGGGGCTCACGGTGCTGAGCA
AGTTTCCTTCTTCAGATATGCCCATTAAATTTGATAAGATCCTGTATAACCAATTCAACCATTATGATACAGC
AGCGGGGAAATTCACGTGCCACATTGCTGGGGTCTATTACTTCACCTACCACATCACTGTTTTCTCCAGAAAT
GTTCAGGTGTCTTTGGTCAAAAATGGAGTAAAAATACTGCACACCAAAGATGCTTACATGAGCTCTGAGGACC
AGGCCTCTGGCGGCATTGTCCTCCAGCTGAAGCTCGGGGATGAGGTGTGGCTGCAGGTGACAGGAGGAGAGAG
GTTCAATGGCTTGTTTGCTGATGAGGACGGTGACACAACTTTCACAGGGTTCCTTCTGTTCAGCAGCCCGTGA
CAGAGGAGAGTTTAAAAATCCGCCACCACCATCCATCAGAATCAGCTTGGATGAACTTATTCAGATGGTTTTA
CTTTATTAATTCCTC+TZ,1/46

| | | |
|---|---|---|
| NOV17s, SNP13381822 of | SEQ ID NO: 266 | MW at 24887.9kD |

| | | | |
|---|---|---|---|
| CG95430-01, Protein Sequence | SNP Pos: 218 | 231 aa | SNP Change: Asp to Gly |

NRIWWLLLAIEICTGNINSQDTCRQGHPGIPGNPCHNGLPGRDGRDGAKGDKCDAGKPGPKGEAGPTGPQGEP
GVRGIRGWKGDRGEKGKIGETLVLPKSAFTVGLTVLSKFPSSDMPIKFDKILYNEFNHYDTAAGKFTCHIAGV
YYFTYHITVFSRNVQVSLVKNGVKILHTKDAYNSSEDQASGGIVLQLKLGDEVWLQVTGGERFNGLFADEDGD
TTFTGFLLFSSP

| | |
|---|---|
| SEQ ID NO: 267 | 818 bp |

| | | |
|---|---|---|
| NOV17t, SNP13381826 of | ORF Start: ATG at 35 | ORF Stop: TGA at 728 |

| | | |
|---|---|---|
| CG95430-01, DNA Sequence | SNP Pos: 717 | SNP Change: T to C |

TCCCTCTTTCAGTTCAGAGTCTGTCATCTGAACCATGAGGATCTGGTGGCTTCTGCTTGCCATTGAAATCTGC
ACAGGGAACATAAACTCACAGGACACCTGCAGGCAACGGCACCCTGGAATCCCTGGGAACCCCGGTCACAATG
GTCTCCCTGGAAGAGATGGACGAGACGGAGCGAAGGGTGACAAAGGCGATGCAGGTAAGCCTGGTCCCAAAGG
AGAAGCTGGACCCACGGGGCCCCAGGGTGAGCCAGGAGTCCGGGGAATAAGAGGCTGGAAAGGAGATCGAGGA
GAGAAAGGGAAAATCGGTGAGACTCTAGTCTTGCCAAAAAGTCCTTTCACTGTGGGGCTCACGGTGCTGAGCA
AGTTTCCTTCTTCAGATATGCCCATTAAATTTGATAAGATCCTGTATAACGAATTCAACCATTATGATACAGC
AGCGGGGAAATTCACGTGCCACATTGCTGGGGTCTATTACTTCACCTACCACATCACTGTTTTCTCCAGAAAT
GTTCAGGTGTCTTTGGTCAAAAATCGAGTAAAAATACTGCACACCAAAGATGCTTACATGAGCTCTGAGGACC
AGGCCTCTGGCGGCATTGTCCTGCAGCTGAAGCTCGGGGATCAGGTGTGGCTGCAGGTGACAGGAGGAGAGAG
GTTCAATGGCTTGTTTGCTGATGAGGACGATGACACAACTTTCACAGGGTTCCTTCTGTCCAGCAGCCCGTGA
CAGAGGAGAGTTTAAAAATCCGCCACACCATCCATCAGAATCAGCTTGGGATGAACTTATTCAGATGGTTTTA
CTTTATTAATTCCTC+TZ,1/46

| | | |
|---|---|---|
| NOV17t, SNP13381826 of | SEQ ID NO: 268 | MW at 24885.9kD |

| | | | |
|---|---|---|---|
| CG95430-01, Protein Sequence | SNP Pos: 228 | 231 aa | SNP Change: Phe to Ser |

MRIWWLLLAIEICTGNINSQDTCRQGHPGIPGNPGHNGLPGRDGRDGAKGDKGDAGKPGPKGEAGPTGPQGEP
GVRGIRGWKGDRGEKGKIGETLVLPKSAFTVGLTVLSKFPSSDMPIKFDKILYNEFNHYDTAAGKFTCHIAGV
YYFTYHITVFSRNVQVSLVKNGVKILHTKDAYMSSEDQASGGIVLQLKLGDEVWLQVTGGERFNGLFADEDDD
TTFTGFLLSSSP+TZ,1/46

A ClustalW comparison of the above protein sequences yields the following sequence alignment shown in Table 17B.

TABLE 17B
Comparison of the NOV17 protein sequences.

| | |
|---|---|
| NOV17a | --------------------------QDTCRQGHPGIPGNPGHNGLPGRDGRDGAKGDKG |
| NOV17b | -------MRIWWFLLAIEICTGNINSQDTCRQGHPGIPGNPGHNGLSGRDGRDGAKGDKG |
| NOV17c | -------MRIWWLLLAIEICTGNINSQDTCRQGHPGIPGNPGHNGLPGRDGRDGAKGDKG |
| NOV17d | ---TGSTMRIWWFLLAIEICTGNINSQDTCRQGHPGIPGNPGHNGLPGRDGRDGAKGDKG |
| NOV17e | ------------------------------------------------------------ |
| NOV17f | -------MRIWWLLLAIEICTGNINSQDTCRQGHPGIPGNPGHNGLPGRDGRDGAKGDKG |
| NOV17g | -------MRIWWFLLAIEICTGNINSQDTCRQGHPGIPGNPGHNGLSGRDGRDGAKGDKG |
| HOV17h | --------------------------QDTCRQGHPGIPGNPGHNGLPGRDGRDGAKGDKG |
| NOV17i | -------MRIWWFLLAIEICTGNINSQDTCRQGNPGIPGNPGHNGLPGRDGRDGAKGDKG |
| NOV17j | --------------------------QDTCRQGHPGIPGNPGHNGLPGRDGRDGAKGDKG |
| NOV17k | -------MRIWWFLLAIEICTGNINSQDTCRQGHPGIPGNPGHNGLPGRDGRDGAKGDKG |
| NOV17l | MGHHHHHHRIWWFLLAIEICTGNINSQDTCRQGHPGIPGNPGHNGLPGRDGRDGAKCDKG |
| NOV17m | --------------------HHHHHHQDTCRQGHPGIPGNPGHNGLPGRDGRDGAKGDKG |
| NOV17n | --------------------HHHHHHQDTCRQGHPGIPGNPGHNGLPGRDGRDGAKGDKG |
| NOV17a | DAGEPGRPGSPGKDGTSGEKGERGADGKVEAKGIKGDQGSRGSPGKHGPKGLAGPMGEKG |
| NOV17b | DAGEPGRPGSPGKDGTSGEKGERGADGKVEAKGIKGDQGSRGSPGKHGPKGLAGPMGEKG |
| NOV17c | DAGKPG------------------------------------------PKGEAGPTG--- |
| NOV17d | DAGEPGRPGSPGKDGTSGEKGERGADGKVEAKGIKGDQGSRGSPGKHGPKGLAGPMGEKG |
| NOV17e | ------------------------------------------------------------ |
| NOV17f | DAGEPGRPGSPGKDGTSGEKGERGADGKVEAKGIKGDQGSRGSPGKHGPKGLAGPMGEKG |
| NOV17g | DAGEPGRPGSPGKDGTSGEKGERGADGKVEAKGIKGDQGSRGSPGKHGPKGLAGPMGEKG |
| NOV17h | DAGEPGRPGSPGKDGTSGEKGERGADGKVEAKCIKGDQGSRGSPGKHGPKGLAGPMGEKG |
| NOV17i | DAGEPGRPGSPGKDGTSGEKGERGADGKVEAKGIKGDQGSRGSPGKHGPKGLAGPMGEKG |
| NOV17j | DAGEPGRPGSPGKDGTSGEKGERGADGKVEAKGIKGDQGSRGSPGKHGPKGLAGPMGEKG |
| NOV17k | DAGEPGRPGSPGKDGTSGEKGERGADGKVEAKGIKGDQGSRGSPGKHGPKGLAGPMGEKG |
| NOV17l | DAGEPGRPGSPGKDGTSGEKGERGADGKVEAKGIKGDQGSRGSPGKHGPKGLAGPMGEKG |
| NOV17m | DAGEPGRPGSPGKDGTSGEKGERCADGKVEAKGIKGDQGSRGSPGKHGPKGLAGPMGEKG |
| NOV17n | DAGEPGRPGSPGKDGTSGEKGERGADGKVEAKGIKGDQGSRGSPGKHGPKGLAGPMGEKG |
| NOV17a | LRGETGPQGQKGNKGDVGPTGPEGPRGNIGPLGPTGLPGPMGPIGKPGPKGEAGPTGPQG |
| NOV17b | LRGETGPQGQKGNKGDVGPTGPEGPRGNIGPLGPTGLPGPMGPIGKPGPKGEAGPTGPQG |
| NOV17c | ------PQG--------------------------------------------------- |
| NOV17d | LRGETGPQGQKGNKGDVGPTGPEGPRGNIGPLGPTGLPGPMGPIGKPGPKGEAGPTGPQG |
| NOV17e | ------------------------------------------------------------ |
| NOV17f | LRGETGPQGQKGNKGDVGPTGPEGPRGNIGPLGPTGLPGPMGPIGKPGPKGEAGPTGPQG |
| NOV17g | LRGETGPQGQKGNKG--------------------------------------------- |
| NOV17h | LRGETGPQGQKGNKGDVGPTGPEGPRGNIGPLGPTGLPGPMGPIGKPGPKGEAGPTGPQG |
| NOV17i | LRGETGPQGQKGNKGDVGPTGPEGPRGNIGPLGPTGLPGPMGPIGKPGPKGEAGPTGPQG |
| NOV17j | LRGETGPQGQKGNKGDVGPTGPEGPRGNIGPLGPTGLPGPMGPIGKPGPKGEAGPTGPQG |
| NOV17k | LRGETGPQGQKGNKCDVGPTGPEGPRGNIGPLGPTGLPGPMGPIGKPGPKGEAGPTGPQG |
| NOV17l | LRGETGPQGQKGNKGDVGPTGPEGPRGNIGPLGPTGLPGPNGPIGKPGPKGEAGPTGPQG |
| NOV17m | LRGETGPQGQKGNKGDVGPTGPEGPRGNIGPLGPTGLPGPMGPIGKPGPKGEAGPTGPQG |
| NOV17n | LRGETGPQGQKGNKGDVGPTGPEGPRGNIGPLGPTGLPGPMGPIGKPGPKGEAGPTGPQG |
| NOV17a | EPGVRGIRGWKGDRGEKGKIGETLVLPKSAFTVGLTVLSKFPSSDVPIKFDKILYNEFNH |
| NOV17b | EPGVRGIRGWKGDRGEKGKIGETLVLPKSAFTVGLTVLSKFPSSDMPIKFDKILYNEFNH |
| NOV17c | EPGVRGIRGWKGDRGEKGKIGETLVLPKSAFTVGLTVLSKFPSSDMPIKFDKILYNEFNH |
| NOV17d | EPGVRGIRGWKGDRGEKGKIGETLVLPKSAFTVGLTVLSKFPSSDVPIKFDKILYNEFNH |
| NOV17e | -----------------------------AFTVGLTVLSKFPSSDMPIKFDKILYNEFNH |
| NOV17f | EPGVRGIRGWKGDRGEKGKIGETLVLPKSAFTVGLTVLSKFPSSDVPIKFDK-------- |
| NOV17g | EPGVRGIRGWKGDRGEKGKIGETLVLPKSAFTVGLTVLSKFPSSDVPIKFDKILYNEFNH |
| NOV17h | EPGVRGIRGWKGDRGEKGKIGETLVLPKSAFTVGLTVLSKFPSSDVPIKFDKILYNEFNH |
| NOV17i | EPGVRGIRGWKGDRGEKGKIGETLVLPKSAFTVGLTVLSKFPSSDVPIKFDKILYNEFNH |
| NOV17j | EPGVRGIRGWKGDRGEKGKIGETLVLPKSAFTVGLTVLSKFPSSDVPIKFDKILYNEFNH |
| NOV17k | EPGVRGIRGWKGDRGEKGKIGETLVLPKSAFTVGLTVLSKFPSSDVPIKFDKILYNEFNH |
| NOV17l | EPGVRGIRGWKGDRGEKGKIGETLVLPKSAFTVGLTVLSKFPSSDVPIKFDKILYNEFNH |
| NOV17m | EPGVRGIRGWKGDRGEKGKIGETLVLPKSAFTVGLTVLSKFPSSDVPIKFDKILYNEFNH |
| NOV17n | EPGVRGIRGWKGDRGEKGKIGETLVLPKSAFTVGLTVLSKFPSSDVPIKFDKILYNEFNH |
| NOV17a | YDTAAGKFTCHIAGVYYFTYHITVFSRNVQVSLVKNGVKILHTKDAYMSSEDQASGGIVL |
| NOV17b | YDTAAGKFTCHIAGVYYFTYHITVFSRNVQVSLVKNGVKILHTKDAYMSSEDQASGGIVL |
| NOV17c | YDTAAGKPTCHIAGVYYFTYHITVFSRNVQVSLVKNGVKILHTKDAYMSSEDQASGGIVL |
| NOV17d | YDTAAGKFTCHIAGVYYFTYHITVFSRNVQVSLVKNGVKILHTKDAYMSSEDQASGGIVL |
| NOV17e | YDTAAGKFTCHIAGVYYFTYHITVFSRNVQVSLVKNGVKILHTKDAYMSSEDQASGGIVL |
| NOV17f | --------------I-----HITVFSRNVQVSLVKNGVKILHTRDAYVSSEDQASGSIVL |
| NOV17g | YDTAAGKFTCHIAGVYYFTYHIAVFSSNVQVSLVKNGVKILHTKDAYMSSEDQASGGIVL |
| NOV17h | YDTAAGKFTCHIAGVYYFTYHITVFSRNVQVSLVKNGVKILHTKDAYMSSEDQASGGIVL |
| NOV17i | YDTAAGKFTCHIAGVYYFTYHITVFSRNVQVSLVKNGVKILHTKDAYMSSEDQASGGIVL |
| NOV17j | YDTAAGKFTCNIAGVYYFTYHITVFSRNVQVSLVKNGVKILHTKDAYMSSEDQASGGIVL |
| NOV17k | YDTAAGKFTCHIAGVYYFTYHITVFSRNVQVSLVKNGVKILHTKDAYMSSEDQASGGIVL |
| NOV17l | YDTAAGKFTCHIAGVYYFTYHITVFSRNVQVSLVKNGVKILHTKDAYMSSEDQASGGIVL |
| NOV17m | YDTAAGKFTCHIAGVYYFTYHITVFSRNVQVSLVKNGVKILHTKDAYMSSEDQASGGIVL |
| NOV17n | YDTAAGKFTCHIAGVYYFTYHITVFSRNVQVSLVKNGVKILHTKDAYMSSEDQASGGIVL |
| NOV17a | QLKLGDEVWLQVTGGERFNGLFADEDDDTTFTGFLLFSSP |
| NOV17b | QLKLGDEVWLQVTGGERFNGLFADEDDDTTFTGFLLFSSQ |
| NOV17c | QLKLGDEVWLQVTGGERFNGLFADEDDDTTFTGFLLFSSP |
| NOV17d | QLKLGDEVWLQVTGGEKFNGLFADEDDDTTFTGFLLFSSP |
| NOV17e | QLKLGDEVWLQVTGGERFNGLFADEDDDTTFTGFLLFSSP |
| NOV17f | QLKLGDEMWLQVTGGERFNGLFADEDDDTTFTGFLLFSSQ |
| NOV17g | QLKLGDEVWLQVTGGERFNGLFADEDDDTTFTGFLLFSSQ |
| NOV17h | QLKLGDEVWLQVTGGERFNGLFADEDDDTTFTGFLLFSSP |
| NOV17i | QLKLGDEVWLQVTGGEKFNGLFADEDDDTTFTGFLLFSSP |
| NOV17j | QLKLGDEVWLQVTGGERFNGLFADEDDDTTFTGFLLFSSP |
| HOV17k | QLKLGDEVWLQVTGGEKFNGLFADEDDDTTFTGFLLFSSP |
| NOV17l | QLKLGDEVWLQVTGGEKFNGLFADEDDDTTFTGFLLFSSP |
| NOV17m | QLKLGDEVWLQVTGGEKFNGLFADEDDDTTFTGFLLFSSP |
| NOV17n | QLKLGDEVWLQVTGGERFNGLFADEDDDTTFTGFLLFSSP |
| NOV17a | (SEQ ID NO: 230) |
| NOV17b | (SEQ ID NO: 232) |
| NOV17c | (SEQ ID NO: 234) |
| NOV17d | (SEQ ID NO: 236) |
| NOV17e | (SEQ ID NO: 238) |
| NOV17f | (SEQ ID NO: 240) |
| NOV17g | (SEQ ID NO: 242) |
| NOV17h | (SEQ ID NO: 244) |
| NOV17i | (SEQ ID NO: 246) |
| NOV17j | (SEQ ID NO: 248) |
| NOV17k | (SEQ ID NO: 250) |
| NOV17l | (SEQ ID NO: 252) |
| NOV17m | (SEQ ID NO: 254) |
| NOV17n | (SEQ ID NO: 256) |

Further analysis of the NOV17a protein yielded the following properties shown in Table 17C.

TABLE 17C
Protein Sequence Properties NOV17a

| SignalP analysis: | No Known Signal Sequence Predicted | | | |
|---|---|---|---|---|
| PSORT II | PSG: a new signal peptide prediction method | | | |
| analysis: | N-region: length 5; pos. chg 1; neg. chg 1 | | | |
| | H-region: length 17; peak value 0.39 | | | |
| | PSG score: −4.01 | | | |
| | GvH: von Heijne's method for signal seq. recognition | | | |
| | GvH score (threshold: −2.1): −14.34 | | | |
| | possible cleavage site: between 52 and 53 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 0 | | | |
| | number of TMS(s) . . . fixed | | | |
| | PERIPHERAL | Likelihood = 2.17 (at 177) | | |
| | ALOM score: 2.17 (number of TMSs: 0) | | | |
| | MITDISC: discrimination of mitochondrial targeting seq | | | |
| | R content: | 2 | Hyd Moment (75): | 8.45 |
| | Hyd Moment(95): | 12.46 | G content: | 6 |
| | D/E content: | 2 | S/T content: | 1 |

```
Score: −6.71
Gavel: prediction of cleavage sites for mitochondrial preseq
R-2 motif at 15 CRQ|GH
NUCDISC: discrimination of nuclear localization signals
pat4: none
pat7: none
bipartite: none
content of basic residues: 12.4%
NLS Score: −0.47
KDEL: ER retention motif in the C-terminus: none
ER Membrane Retention Signals:
XXRR-like motif in the N-terminus: DTCR
none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 76.7
COIL: Lupas's algorithm to detect coiled-coil regions
total: 0 residues
Final Results (k = 9/23):
56.5%: cytoplasmic
21.7%: nuclear
  8.7%: mitochondrial
  4.3%: Golgi
  4.3%: vacuolar
  4.3%: plasma membrane
>> prediction for CG95430-02 is cyt (k = 23)
```

A search of the NOV17a protein against the Geneseq database, a proprietary database that contains sequences published in patents and patent publication, yielded several homologous proteins shown in Table 17D.

TABLE 17D
Geneseq Results for NOV17a

| | | NOV17a | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| AAO16580 | Energen-related secreted protein - | 1 . . . 314 | 313/314 (99%) | 0.0 |
| | C2P - Unidentified, 333 aa. | 20 . . . 333 | 314/314 (99%) | |
| | [WO2003009865-A1, 06 FEB. 2003] | | | |
| AAO16571 | C2P secreted protein - Unidentified, | 1 . . . 314 | 313/314 (99%) | 0.0 |
| | 333 aa. [WO2003009861-A1, | 20 . . . 333 | 314/314 (99%) | |
| | 06 FEB. 2003] | | | |
| ABB80582 | Human sbg1033026C1q protein #1 - | 1 . . . 314 | 313/314 (99%) | 0.0 |
| | Homo sapiens, 333 aa. | 20 . . . 333 | 314/314 (99%) | |
| | [WO200222802-A1, 21 MAR. 2002] | | | |
| AAE28184 | Human GMG-3 protein - Homo | 1 . . . 314 | 313/314 (99%) | 0.0 |
| | sapiens, 333 aa. [WO200266505-A2, | 20 . . . 333 | 314/314 (99%) | |
| | 29 AUG. 2002] | | | |
| AAE28185 | Human GMG-4 protein - Homo | 1 . . . 313 | 307/313 (98%) | 0.0 |
| | sapiens, 333 aa. [WO200266505-A2, | 20 . . . 332 | 310/313 (98%) | |
| | 29 AUG. 2002] | | | |

In a BLAST search of public sequence databases, the NOV17a protein was found to have homology to the proteins shown in the BLASTP data in Table 17E.

TABLE 17E
Public BLASTP Results for NOV17a

| | | NOV17a | | |
|---|---|---|---|---|
| Protein | | Residues/ | Identities/ | |
| Accession | | Match | Similarities for the | Expect |
| Number | Protein/Organism/Length | Residues | Matched Portion | Value |
| Q8IUU4 | Hypothetical protein - Homo | 1 . . . 314 | 314/314 (100%) | 0.0 |
| | sapiens (Human), 333 aa. | 20 . . . 333 | 314/314 (100%) | |
| CAD57042 | Sequence 1 from Patent | 1 . . . 314 | 313/314 (99%) | 0.0 |
| | WO02066505 - Homo sapiens | 20 . . . 333 | 314/314 (99%) | |
| | (Human), 333 aa. | | | |
| CAD57043 | Sequence 3 from Patent | 1 . . . 313 | 307/313 (98%) | 0.0 |
| | WO02066505 - Homo sapiens | 20 . . . 332 | 310/313 (98%) | |
| | (Human), 333 aa. | | | |
| CAD57045 | Sequence 7 from Patent | 1 . . . 313 | 267/313 (85%) | e−164 |
| | WO02066505 - Mus musculus | 20 . . . 329 | 284/313 (90%) | |
| | (Mouse), 330 aa. | | | |
| CAD57046 | Sequence 9 from Patent | 1 . . . 313 | 260/313 (83%) | e−158 |
| | WO02066505 - Mus musculus | 20 . . . 322 | 277/313 (88%) | |
| | (Mouse), 323 aa. | | | |

PFam analysis predicts that the NOV17a protein contains the domains shown in the Table 17F.

TABLE 17F
Domain Analysis of NOV17a

| | | Identities/ | |
|---|---|---|---|
| | NOV17a | Similarities | Expect |
| Pfam Domain | Match Region | for the Matched Region | Value |
| Collagen | 5 . . . 63 | 29/60 (48%) | 8.8e−11 |
| | | 45/60 (75%) | |
| Collagen | 65 . . . 123 | 25/60 (42%) | 2.2e−07 |
| | | 40/60 (67%) | |
| Collagen | 124 . . . 183 | 28/60 (47%) | 0.00021 |
| | | 38/60 (63%) | |
| C1q | 184 . . . 310 | 58/141 (41%) | 7.2e−42 |
| | | 98/141 (70%) | |

### Example 18

The NOV18 clone was analyzed, and the nucleotide and encoded polypeptide sequences are shown in Table 18A.

TABLE 18A
NOV18 Sequence Analysis

| | | |
|---|---|---|
| NOV18a, CG97111-01 | SEQ ID NO: 269 | 1019 bp |

| | | |
|---|---|---|
| DNA Sequence | ORF Start: ATG at 54 | ORF Stop: TAG at 531 |

GGTTCCAGGAACTCAGGATCTGCAGTGAGGACCAGACACCACTGATTGCAGGAATGTGTTCCCTCCCCATGGC
AAGATACTACAGAATTAAATATGCAGACCAGAAGCCTCTATACACAAGAGATGGCCAGCTGCTGGTGGGAGAT
CCTGTTGCAGACAACTGCTGTGCAGAGAAGATCTGCATACTTCCTAACAGAGGCTTGGCCCGCACCAAGGTCC
CCATTTTCCTGGGGATCCAGGGAGGGAGCCGCTGCCTGGCATGTGTGGAGACACAAGAGGGGCCTTCCCTACA
GCTGGAGCCATCCACCTTGCCCCCACAGGATGTGAACATTGAGGAACTGTACAAAGGTGGTGAAGAGGCCACA
CGCTTCACCTTCTTCCAGAGCACCTCAGGCTCCGCCTTCAGGCTTGAGGCTGCTGCCTGGCCTGGCTGGTTCC
TGTGTGGCCCGGCAGAGCCCCAGCAGCCAGTACAGCTCACCAAGGAGAGTGAGCCCTCAGCCCGTACCAAGTT
TTACTTTGAACAGAGCTGGTAGGGAGACAGGAAACTGCCTTTTAGCCTTGTGCCCCCAAACCAAGCTCATCCT
GCTCAGGGTCTATGGTAGGCAGAATAATGTCCCCCGAAATATGTCCACATCCTAATCCCAAGATCTGTGCATA
TGTTACCATACATGTCCAAAGAGGTTTTGCAAATGTGATTATGTTAAGGATCTTGAAATGAGGAGACAATCCT
CGGTTATCCTTGTGGGCTCAGTTTAATCACAAGAAGGAGGCAGGAAGGGAGAGTCAGAGAGAGAATGGAAGAT
ACCATGCTTCTAATTTTGAAGATGGAGTGAGGGGCCTTGAGCCAACATATGCAGGTGTTTTTAGAAGGAGGAA
AACCCAAGGGAACGGATTCTCCTCTATAGTCTCCGGAAGGAACACAGCTCTTGACACATGGATTTCAGCTCAG
TGACACCCATTTCAGACTTCTGACCTCCACAACTATAAAATAATAAACTTGTGTTATTGTAAACCTCTGG+TZ,1/46

NOV18a, CG97111-01

| | | | |
|---|---|---|---|
| Protein Sequence | SEQ ID NO: 270 | 159 aa | MW at 17706.9kD |

MCSLPMARYYRIKYADQKALYTRDGQLLVGDPVADNCCAEKICILPNRGLARTKVPIFLGIQGGSRCLACVET
EEGPSLQLEPSTLPPQDVNIEELYKCGEEATRFTFFQSSSGSAFRLEAAAWPGWFLCGPAEPQQPVQLTKESE
PSARTKFYFEQSW

| | | |
|---|---|---|
| NOV18b, CG97111-02 | SEQ ID NO:271 | 499 bp |

| | | |
|---|---|---|
| DNA Sequence | ORF Start: ATG at 16 | ORF Stop: TAG at 472 |

CCACTGATTGCAGGAATGTGTTCCCTCCCCATGGCAAGATACTACATAATTAAATATGCAGACCAGAAGGCTC
TATACACAAGAGATGGCCACCTGCTCGTGGGAGATCCTGTTGCAGACAACTGCTGTGCAGAGAAGATCTGCAT
ACTTCCTAACAGAGGCTTGGCCCGCACCAAGGTCCCCATTTTCCTGGGGATCCAGGGAGGGAGCCGCTGCCTG
GCATGTGTGGACACAGAAGAGGGGCCTTCCCTACAGCTGGAGGATGTGAACATTGAGGAACTGTACAAAGGTG
GTGAAGAGGCCACACGCTTCACCTTCTTCCAOAGCAGCTCAGGCTCCGCCTTCAGGCTTGAGGCTGCTGCCTG
GCCTCGCTGGTTCCTGTGTGGCCCGGCAGAGCCCCAGCAGCCAGTACAGCTCACCAAGGACAGTGAGCCCTCA
GCCCGTACCAAGTTTTACTTTGAACAGAGCTGGTAGGGAGACAGGAAACTGCGTTTTAGCC+TZ,1/46

NOV18b, CG97111-02

| | | | |
|---|---|---|---|
| Protein Sequence | SEQ ID NO: 272 | 152 aa | MW at 16943.1kD |

MCSLPMARYYIIKYADQKALYTRDGQLLVGDPVADNCCAEKICILPNRGLARTKVPIFLGIQGGSRCLACVET
EEGPSLQLEDVNIEELYKGGEEATRFTFFQSSSGSAFRLEAAAWPGWFLCGPAEPQQPVQLTKESEPSARTKF
YFEQSW

| | | |
|---|---|---|
| NOV18c, CG97111-03 | SEQ ID NO: 273 | 483 bp |

| | | |
|---|---|---|
| DNA Sequence | ORF Start: at 3 | ORF Stop: TAG at 465 |

CACTGTCATACTGTTTCAGAATTAAATATGCAGACCAGAAGGCTCTATACACAAGAGATGGCCAGCTGCTGGT
GGGAGATCCTGTTGCAGACAACTGCTGTGCAGAGAAGATCTGCATACTTCCTAACAGAGGCTTGGCCCGCACC
AAGGTCCCCATTTTCCTGGGGATCCAGGGAGGGAGCCGCTGCCTGGCATGTGTGGAGACAGAAGAGGGGCCTT
CCCTACAGCTGGAGCCATCCACCTTGCCCCCACAGGATGTGAACATTGAGCAACTGTACAAAGGTGGTCAAGA
GGCCACACGCTTCACCTTCTTCCAGAGCAGCTCAGGCTCCGCCTTCAGGCTTGAGGCTGCTGCCTGGCCTGGC
TGGTTCCTGTGTGGCCCGGCAGAGCCCCAGCAGCCAGTACAGCTCACCAAGGAGAGTGAGCCCTCAGCCCGTA
CCAAGTTTTACTTTGAACAGAGCTGGTAGGGAGACAGGAAACTGC+TZ,1/46

NOV18c, CG97111-03

| | | | |
|---|---|---|---|
| Protein Sequence | SEQ ID NO: 274 | 154 aa | MW at 17104.2kD |

LSYCFRIKYADQKALYTRDGQLLVGDPVADNCCAEKICILPNRGLARTKVPIFLGIQGGSRCLACVETEEGPS
LQLEPSTLPPQDVNIEELYKGGEEATRFTFFQSSSGSAFRLEAAAWPGWFLCGPAEPQQPVQLTKESEPSART
KFYFEQSW

| | |
|---|---|
| SEQ ID NO: 275 | 1019 bp |

| | | |
|---|---|---|
| NOV18d, SNP13382516 of | ORF Start: ATG at 54 | ORF Stop: TAG at 531 |

| | | |
|---|---|---|
| CG97111-01, DNA Sequence | SNP Pos: 125 | SNP Change: T to C |

GGTTCCAGGAACTCAGCATCTGCAGTGACGACCAGACACCACTGATTGCAGGAATGTGTTCCCTCCCCATGGC
AAGATACTACAGAATTAAATATGCAGACCAGAAGGCTCTATACACAAGAGACGGCCAGCTGCTGGTGGGAGAT
CCTGTTGCAGACAACTGCTGTGCAGAGAAGATCTGCATACTTCCTAACAGAGGCTTGGCCCGCACCAAGGTCC
CCATTTTCCTGGGCATCCAGGGAGGGAGCCGCTGCCTGGCATGTGTGGAGACAGAAGAGGGGCCTTCCCTACA
GCTCGAGCCATCCACCTTGCCCCCACAGGATGTGAACATTGAGGAACTGTACAAAGGTGGTGAAGAGGCCACA
CGCTTCACCTTCTTCCAGAGCAGCTCAGGCTCCGCCTTCAGGCTTGAGGCTGCTGCCTGGCCTGGCTGGTTCC
TGTGTGGCCCGCCAGAGCCCCAGCAGCCAGTACAGCTCACCAAGGAGAGTGAGCCCTCAGCCCGTACCAAGTT
TTACTTTGAACAGAGCTGGTAGGGAGACAGGAAACTGCCTTTTAGCCTTGTGCCCCCAAACCAAGCTCATCCT
GCTCAGGGTCTATGGTAGGCAGAATAATGTCCCCCGAAATATGTCCACATCCTAATCCCAAGATCTGTGCATA
TCTTACCATACATGTCCAAAGAGGTTTTGCAAATGTGATTATGTTAAGGATCTTGAAATGAGGAGACAATCCT
GGGTTATCCTTGTGGGCTCAGTTTAATCACAAGAAGGAGGCAGGAAGGGAGAGTCAGAGAGAGAATGGAAGAT
ACCATGCTTCTAATTTTGAAGATGGAGTGAGCGGCCTTGACCCAACATATGCAGGTGTTTTTAGAAGGAGGAA
AAGCCAAGGGAACGGATTCTCCTCTATAGTCTCCGGAAGGAACACAGCTCTTGACACATGGATTTCAGCTCAG
TCACACCCATTTCAGACTTCTGACCTCCACAACTATAAAATAATAAACTTGTGTTATTGTAAACCTCTGG+TZ,1/46

| | | |
|---|---|---|
| NOV18d, SNP13382516 of | SEQ ID NO: 276 | MW at 17706.9kD |

| | | | |
|---|---|---|---|
| CG97111-01, Protein Sequence | SNP Pos: 24 | 159 aa | SNP Change: Asp to Asp |

MCSLPMARYYRIKYADQKALYTRDGQLLVGDPVADNCCAEKICILPNRGLARTKVPIFLGIQGGSRCLACVET
EEGPSLQLEPSTLPPQDVNIEELYKGGEEATRFTFFQSSSGSAFRLEAAAWPGWFLCGPAEPQQPVQLTKESE
PSARTKFYFEQSW

| | |
|---|---|
| SEQ ID NO: 277 | 1019 bp |

| | | |
|---|---|---|
| NOV18e, SNP13382517 of | ORF Start: ATG at 54 | ORF Stop: TAG at 531 |

| | | |
|---|---|---|
| CG97111-01, DNA Sequence | SNP Pos: 184 | SNP Change: T to C |

GGTTCCAGGAACTCAGGATCTGCAGTGAGGACCAGACACCACTGATTGCAGGAATGTGTTCCCTCCCCATGGC
AAGATACTACAGAATTAAATATGCAGACCAGAAGGCTCTATACACAAGAGATGGCCAGCTGCTGGTGGGAGAT
CCTGTTGCAGACAACTGCTGTGCAGAGAAGATCTGCACACTTCCTAACAGAGGCTTGGCCCGCACCAACGTCC
CCATTTTCCTGGCGATCCAGGCAGGGAGCCGCTGCCTGGCATGTGTGGAGACAGAAGAGGGGCCTTCCCTACA
GCTGGAGCCATCCACCTTGCCCCCACAGGATGTGAACATTGAGGAACTGTACAAAGGTGGTGAAGAGGCCACA
CGCTTCACCTTCTTCCAGAGCAGCTCAGGCTCCGCCTTCAGGCTTGAGGCTGCTGCCTGGCCTGGCTGGTTCC
TGTGTGGCCCGGCAGAGCCCCAGCAGCCAGTACAGCTCACCAAGGAGAGTGAGCCCTCAGCCCGTACCAAGTT
TTACTTTGAACAGAGCTGGTAGGGAGACAGGAAACTGCGTTTTAGCCTTGTGCCCCCAAACCAAGCTCATCCT
GCTCAGGGTCTATGGTAGGCAGAATAATGTCCCCCGAAATATGTCCACATCCTAATCCCAAGATCTGTGCATA
TGTTACCATACATGTCCAAAGAGGTTTTGCAAATGTGATTATGTTAAGGATCTTGAAATCAGGAGACAATCCT
GGGTTATCCTTGTGGGCTCAGTTTAATCACAAGAAGGAGGCAGGAAGGGAGAGTCAGAGAGAGAATGGAAGAT
ACCATGCTTCTAATTTTGAAGATGGAGTGAGGGGCCTTGAGCCAACATATGCAGGTGTTTTTAGAAGGAGGAA
AAGCCAAGGCAACGGATTCTCCTCTATAGTCTCCGGAAGGAACACAGCTCTTGACACATGGATTTCAGCTCAG
TGACACCCATTTCAGACTTCTGACCTCCACAACTATAAAATAATAAACTTGTGTTATTGTAAACCTCTGG+TZ,1/46

| | | |
|---|---|---|
| NOV18e, SNP13382517 of | SEQ ID NO: 278 | MW at 17694.9kD |

| | | | |
|---|---|---|---|
| CG97111-01, Protein Sequence | SNP Pos: 44 | 159 aa | SNP Change: Ile to Thr |

MCSLPMARYYRIKYADQKALYTRDGQLLVGDPVADNCCAEKICTLPNRGLARTKVPIFLGIQGGSRCLACVET
EEGPSLQLEPSTLPPQDVNIEELYKGGEEATRFTFFQSSSGSAFRLEAAAWPGWFLCGPAEPQQPVQLTKESE
PSARTKFYFEQSW

| | |
|---|---|
| SEQ ID NO: 279 | 1019 bp |

| | | |
|---|---|---|
| NOV18f, SNP13382518 of | ORF Start: ATG at 54 | ORF Stop: TAG at 531 |

| | | |
|---|---|---|
| CG97111-01, DNA Sequence | SNP Pos: 205 | SNP Change: C to A |

GGTTCCAGGAACTCACGATCTGCAGTGAGGACCACACACCACTCATTGCAGGAATGTGTTCCCTCCCCATGGC
AAGATACTACAGAATTAAATATGCAGACCAGAAGGCTCTATACACAAGAGATGGCCAGCTGCTGGTCGGAGAT
CCTGTTGCAGACAACTGCTGTGCAGAGAAGATCTGCATACTTCCTAACAGAGGCTTGGACCGCACCAAGCTCC
CCATTTTCCTGCCGATCCAGCGAGGGAGCCGCTGCCTGGCATGTGTGGAGACAGAAGAGGGGCCTTCCCTACA
GCTGGAGCCATCCACCTTGCCCCCACAGGATGTGAACATTGAGGAACTGTACAAAGGTGGTGAAGAGGCCACA
CGCTTCACCTTCTTCCAGAGCAGCTCACGCTCCGCCTTCAGGCTTGACGCTGCTGCCTGGCCTGGCTGGTTCC
TGTGTGGCCCGGCAGAGCCCCAGCAGCCAGTACAGCTCACCAAGGAGAGTGAGCCCTCAGCCCGTACCAAGTT
TTACTTTGAACAGAGCTGGTAGGGAGACAGGAAACTGCGTTTTAGCCTTGTGCCCCCAAACCAAGCTCATCCT
GCTCAGGGTCTATGGTAGGCAGAATAATGTCCCCCGAAATATGTCCACATCCTAATCCCAAGATCTGTGCATA
TGTTACCATACATGTCCAAACAGGTTTTGCAAATGTGATTATGTTAAGGATCTTGAAATGAGGAGACAATCCT
GGGTTATCCTTGTGGGCTCAGTTTAATCACAAGAAGGAGGCAGGAAGGGAGAGTCAGAGAGAGAATGGAAGAT
ACCATGCTTCTATTTTGAAGATGGAGTGAGGGGCCTTGAGCCAACATATGCAGGTGTTTTTAAGAAGGAGGAA
AAGCCAAGGGAACGGATTCTCCTCTATAGTCTCCGGAAGGAACACAGCTCTTGACACATGCATTTCAGCTCAG
TGACACCCATTTCAGACTTCTGACCTCCACAACTATAAAATAATAAACTTGTGTTATTGTAAACCTCTGG+TZ,1/46

| | | |
|---|---|---|
| NOV18f, SNP13382518 of | SEQ ID NO: 280 | MW at 17750.9kD |

| | | | |
|---|---|---|---|
| CG97111-01, Protein Sequence | SNP Pos: 51 | 159 aa | SNP Change: Ala to Asp |

MCSLPMARYYRIKYADQKALYTRDGQLLVGDPVADNCCAEKICILPNRGLDRTKVPIFLGIQGGSRCLACVET
EEGPSLQLEPSTLPPQDVNILEELYKGGEEATRFTFFQSSSGSAFRLEAAAWPGWFLCGPAEPQQPVQLTKSE
PSARTKFYFEQSW

A ClustalW comparison of the above protein sequences yields the following sequence alignment shown in Table 18B.

TABLE 18B
Comparison of the NOV18 protein sequences.

| | |
|---|---|
| NOV18a | MCSLPMARYYRIKYADQKALYTRDGQLLVGDPVADNCCAEKICILPNRGLARTKVPIFLG |
| NOV18b | MCSLPMARYYIIKYADQKALYTRDGQLLVGDPVADNCCAEKICILPNRGLARTKVPIFLG |
| NOV18c | -----LSYCFRIKYADQKALYTRDGQLLVGDPVADNCCAEKICILPNRGLARTKVPIFLG |
| NOV18a | IQGGSRCLACVETEEGPSLQLEPSTLPPQDVNIEELYKGGEEATRFTFFQSSSGSAFRLE |
| NOV18b | IQGGSRCLACVETEEGPSLQLE-------DVNIEELYKGGEEATRFTFFQSSSGSAFRLE |
| NOV18c | IQGGSRCLACVETEEGPSLQLEPSTLPPQDVNIEELYKGGEEATRFTFFQSSSGSAFRLE |
| NOV18a | AAAWPGWFLCGPAEPQQPVQLTKESEPSARTKFYFEQSW |
| NOV18b | AAAWPGWFLCGPAEPQQPVQLTKESEPSARTKFYFEQSW |
| NOV18c | AAAWPGWFLCGPAEPQQPVQLTKESEPSARTKFYFEQSW |
| NOV18a | (SEQ ID NO: 270) |
| NOV18b | (SEQ ID NO: 272) |
| NOV18c | (SEQ ID NO: 274) |

Further analysis of the NOV18a protein yielded the following properties shown in Table 18C.

TABLE 18C
Protein Sequence Properties NOV18a

| SignalP | No Known Signal Sequence Predicted | | | |
|---|---|---|---|---|
| analysis: | | | | |
| PSORT II | PSG: a new signal peptide prediction method | | | |
| analysis: | N-region: length 11; pos. chg 2; neg. chg 0 | | | |
| | H-region: length 1; peak value −13.22 | | | |
| | PSG score: −17.62 | | | |
| | GvH: von Heijne's method for signal seq. recognition | | | |
| | GvH score (threshold: −2.1): −5.73 | | | |
| | possible cleavage site: between 39 and 40 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 0 | | | |
| | number of TMS(s) . . . fixed | | | |
| | PERIPHERAL | Likelihood = 2.07 (at 55) | | |
| | ALOM score: 2.07 (number of TMSs: 0) | | | |
| | MITDISC: discrimination of mitochondrial targeting seq | | | |
| | R content: | 2 | Hyd Moment(75): | 8.63 |
| | Hyd Moment(95): | 6.96 | G content: | 0 |
| | D/E content: | 1 | S/T content: | 1 |

```
Score: −2.42
Gavel: prediction of cleavage sites for mitochondrial preseq
R-2 motif at 21 YRI|KY
NUCDISC: discrimination of nuclear localization signals
pat 4: none
pat 7: none
bipartite: none
content of basic residues: 10.1%
NLS Score: −0.47
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: found
ILPN at 44
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 55.5
COIL: Lupas's algorithm to detect coiled-coil regions
total: 0 residues
Final Results (k = 9/23):
47.8%: nuclear
39.1%: mitochondrial
  4.3%: vacuolar
  4.3%: vesicles of secretory system
  4.3%: cytoplasmic
>> prediction for CG97111-01 is nuc (k = 23)
```

A search of the NOV18a protein against the Geneseq database, a proprietary database that contains sequences published in patents and patent publication, yielded several homologous proteins shown in Table 18D.

TABLE 18D
Geneseq Results for NOV18a

| | | NOV18a | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| ABP52020 | NOVINTRA A homologous amino | 10 . . . 159 | 149/150 (99%) | 6e−86 |
| | acid sequence SEQ ID NO: 63 - Homo | 9 . . . 158 | 150/150 (99%) | |
| | sapiens, 158 aa. [US2002068279-A1, | | | |
| | 06 JUN 2002] | | | |
| ABP51984 | Human IL-1 receptor antagonist | 10 . . . 159 | 149/150 (99%) | 6e−86 |
| | protein NOVINTRA A SEQ ID NO: 8 - | 5 . . . 154 | 150/150 (99%) | |
| | Homo sapiens, 154 aa. | | | |
| | [US2002068279-A1, 06 JUN. 2002] | | | |
| AAB84999 | Human interleukin-1 receptor | 10 . . . 159 | 149/150 (99%) | 6e−86 |
| | antagonist (NOVINTRA A) | 5 . . . 154 | 150/150 (99%) | |
| | polypeptide - Homo sapiens, 154 aa. | | | |
| | [WO200140291-A2, 07 JUN. 2001] | | | |
| AAU98463 | Novel human interleukin-1 related | 1 . . . 159 | 151/159 (94%) | 1e−84 |
| | polypeptide - Homo sapiens, 152 aa. | 1 . . . 152 | 151/159 (94%) | |
| | [WO200250113-A2, 27 JUN. 2002] | | | |
| AAM50219 | Interleukin-1 receptor antagonist | 1 . . . 159 | 151/159 (94%) | 1e−84 |
| | related protein splice variant - Homo | 20 . . . 171 | 151/159 (94%) | |
| | sapiens, 171 aa. [WO200142304-A1, | | | |
| | 14 JUN. 2001] | | | |

In a BLAST search of public sequence databases, the NOV18a protein was found to have homology to the proteins shown in the BLASTP data in Table 18E.

TABLE 18E
Public BLASTP Results for NOV18a

| | | NOV18a | Identities/ | |
|---|---|---|---|---|
| Protein | | Residues/ | Similarities for | |
| Accession | | Match | the Matched | Expect |
| Number | Protein/Organism/Length | Residues | Portion | Value |
| CAC43507 | Sequence 5 from Patent WO0142304 - | 1 . . . 159 | 151/159 (94%) | 4e−84 |
| | Homo sapiens (Human), 171 aa. | 20 . . . 171 | 151/159 (94%) | |
| Q8WWZ1 | Interleukin 1 family member 10 | 1 . . . 159 | 151/159 (94%) | 4e−84 |
| | (IL-1F10) (Interleukin-1 receptor | 1 . . . 152 | 151/159 (94%) | |
| | antagonist-like FIL1 theta) (Interleukin-1 | | | |
| | theta) (IL-1 theta) (FIL1 theta) | | | |
| | (Interleukin-1 HY2) (IL-1HY2) | | | |
| | (Interleukin-1 receptor antagonist | | | |
| | FKSG75) - Homo sapiens (Human), 152 | | | |
| | aa. | | | |
| CAC21779 | Sequence 3 from Patent WO0071719 - | 1 . . . 159 | 149/159 (93%) | 7e−83 |
| | Homo sapiens (Human), 169 aa | 18 . . . 169 | 149/159 (93%) | |
| | (fragment). | | | |
| CAC21778 | Sequence 1 from Patent WO0071719 - | 1 . . . 159 | 149/159 (93%) | 7e−83 |
| | Homo sapiens (Human), 152 aa. | 1 . . . 152 | 149/159 (93%) | |
| Q8R459 | Interleukin 1 family member 10 | 1 . . . 158 | 123/158 (77%) | 3e−65 |
| | (IL-1F10) - Mus musculus (Mouse), 152 | 1 . . . 151 | 133/158 (83%) | |
| | aa. | | | |

PFam analysis predicts that the NOV18a protein contains the domains shown in the Table 18F.

TABLE 18F
Domain Analysis of NOV18a

| | | Identities/ | |
|---|---|---|---|
| Pfam | | Similarities | Expect |
| Domain | NOV18a Match Region | for the Matched Region | Value |
| IL1 | 12 . . . 159 | 42/153 (27%) | 2e−16 |
| | | 97/153 (63%) | |

### Example 19

The NOV19 clone was analyzed, and the nucleotide and encoded polypeptide sequences are shown in Table 19A.

TABLE 19A

NOV19a, pCR2.1-CG10132038.0.67-S540u2,

| | | |
|---|---|---|
| a domain of CG50513-05 | SEQ ID NO: 281 | 1377 bp |

TGGGAACATAATCCTTGGACTGCATGTTCCGTGTCCTGTGGAGGAGGGATTCAGAGACGGAGCTTTGTGTGTG
TAGAGGAATCCATGCATGGAGAGATATTGCAGGTGGAAGAATGGAAGTGCATGTACGCACCCAAACCCAAGGT
TATGCAAACTTGTAATCTGTTTGATTGCCCCAAGTGGATTGCCATGGAGTGGTCTCAGTGCACAGTGACTTGT
GGCCGAGGCTTACGGTACCGGGTTGTTCTGTGTATTAACCACCGCGGAGAGCATGTTGGGGGCTGCAATCCAC
AACTGAACTTACACATCAAAGAAGAATGTGTCATTCCCATCCCGTGTTATAAACCAAAAGAAAAAAGTCCAGT
GGAAGCAAAATTGCCTTGGCTGAAACAAGCACAAGAACTAGAAGAGACCAGAATAGCAACAGAAGAACCAACG
TTCATTCCAGAACCCTGGTCAGCCTGCAGTACCACGTGTGGGCCGGGTGTGCAGGTCCGTGAGGTGAAGTGCC
GTGTGCTCCTCACATTCACGCAGACTGAGACTGAGCTGCCCGAGGAAGAGTGTGAAGGCCCCAAGCTGCCCAC
CGAACGGCCCTGCCTCCTGGAAGCATGTGATGACAGCCCGGCCTCCCGAGAGCTAGACATCCCTCTCCCTGAG
GACAGTGAGACGACTTACGACTGGGAGTACGCTGGGTTCACCCCTTGCACAGCAACATGCGTGGGACGCCATC
AAGAAGCCATAGCAGTGTGCTTACATATCCAGACCCAGCAGACAGTCAATGACAGCTTGTGTGATATGGTCCA
CCGTCCTCCAGCCATGAGCCAGGCCTGTAACACAGACCCCTGTCCCCCCACGTGGCATGTGGGCTCTTGGGGG
CCCTGCTCAGCTACCTGTGGAGTTGGAATTCAGACCCGAGATGTGTACTGCCTGCACCCAGGGGAGACCCCTC
CCCCTCCTGAGGAGTGCCGAGATGAAAAGCCCCATGCTTTACAAGCATGCAATCAGTTTGACTGCCCTCCTGG
CTGGCACATTGAAGAATGGCAGCAGTGTTCCAGGACTTGTGGCGGGGGAACTCAGAACAGAAGAGTCACCTGT
CGGCAGCTGCTAACGGATGGCAGCTTTTTGAATCTCTCAGATGAATTGTGCCAAGGACCCAAGGCATCGTCTC
ACAAGTCCTGTGCCAGGACAGACTGTCCTCCACATTTAGCTGTGGGAGACTGGTCGAAGTGTTCTGTCAGTTG
TGGTGTTGGAATCCAGAGAAGAAAGCAGGTGTGTCAAAGGCTGGCAGCCAAAGGTCGGCGCATCCCCCTCAGT
GAGATGATGTGCAGGGATCTACCAGGGCTCCCTCTTGTAAGATCTTGCCAGATCCCTGAGTGC

NOV18f, SNP13382518 of

| | | |
|---|---|---|
| CG97111-01, Protein Sequence | SEQ ID NO: 282 | 458 aa |

EHWPWTACSVSCGGGTQRRSFVCVEESMHGEILQVEEWKCMYAPKPKVMQTCNLFDCPKWIAMEWSQCTVTCG
RGLRYRVVLCINHRGEHVGGCNPQLKLHIKEECVIPIPCYKPKEKSPVEAKLPWLKQAQELEETRIATEEPTF
IPEPWSACSTTCGPGVQVREVKCRVLLTFTQTETELPEEECEGPKLPTERPCLLEACDESPASRELDIPLPED
SETTYDWEYAGFTPCTATCVGCHQEAIAVCLHIQTQQTVNDSLCDMVHRPPAMSQACNTEPCPPRWHVGSWGP
CSATCGVGIQTRDVYCLHPGETPAPPEECRDEKPHALQACNQFDCPPGWHIEEWQQCSRTCGGGTQNRRVTCR
QLLTDGSFLNLSDELCQGPKASSHKSCARTDCPPHLAVCDWSKCSVSCGVGIQRRKQVCQRLAAKGRRIPLSE
MMCRDLPGLPLVRSCQMPEC

### Example B

### Sequencing Methodology and Identification of NOVX Clones

1. GeneCalling™ Technology: This is a proprietary method of performing differential gene expression profiling between two or more samples developed at CuraGen and described by Shimkets, et al., “Gene expression analysis by transcript profiling coupled to a gene database query” Nature Biotechnology 17:198-803 (1999). cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then digested with up to as many as 120 pairs of restriction enzymes and pairs of linker-adaptors specific for each pair of restriction enzymes were ligated to the appropriate end. The restriction digestion generates a mixture of unique cDNA gene fragments. Limited PCR amplification is performed with primers homologous to the linker adapter sequence where one primer is biotinylated and the other is fluorescently labeled. The doubly labeled material is isolated and the fluorescently labeled single strand is resolved by capillary gel electrophoresis. A computer algorithm compares the electropherograms from an experimental and control group for each of the restriction digestions. This and additional sequence-derived information is used to predict the identity of each differentially expressed gene fragment using a variety of genetic databases. The identity of the gene fragment is confirmed by additional, gene-specific competitive PCR or by isolation and sequencing of the gene fragment.

2. SeqCalling™ Technology: cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then sequenced using CuraGen's proprietary SeqCalling technology. Sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

3. PathCalling™ Technology: The NOVX nucleic acid sequences are derived by laboratory screening of cDNA library by the two-hybrid approach. cDNA fragments covering either the full length of the DNA sequence, or part of the sequence, or both, are sequenced. In silico prediction was based on sequences available in CuraGen Corporation's proprietary sequence databases or in the public human sequence databases, and provided either the full length DNA sequence, or some portion thereof. The laboratory screening was performed using the methods summarized below:

cDNA libraries were derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then directionally cloned into the appropriate two-hybrid vector (Ga14-activation domain (Ga14-AD) fusion). Such cDNA libraries as well as commercially available cDNA libraries from Clontech (Palo Alto, Calif.) were then transferred from E. coli into a CuraGen Corporation proprietary yeast strain (disclosed in U.S. Pat. Nos. 6,057,101 and 6,083,693, incorporated herein by reference in their entireties).

Ga14-binding domain (Ga14-BD) fusions of a CuraGen Corporation proprietary library of human sequences was used to screen multiple Ga14-AD fusion cDNA libraries resulting in the selection of yeast hybrid diploids in each of which the Ga14-AD fusion contains an individual cDNA. Each sample was amplified using the polymerase chain reaction (PCR) using non-specific primers at the cDNA insert boundaries. Such PCR product was sequenced; sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

Physical clone: the, cDNA fragment derived by the screening procedure, covering the entire open reading frame is, as a recombinant DNA, cloned into pACT2 plasmid (Clontech) used to make the cDNA library. The recombinant plasmid is inserted into the host and selected by the yeast hybrid diploid generated during the screening procedure by the mating of both CuraGen Corporation proprietary yeast strains N106′ and YULH (U.S. Pat. Nos. 6,057,101 and 6,083,693).

4. RACE: Techniques based on the polymerase chain reaction such as rapid amplification of cDNA ends (RACE), were used to isolate or complete the predicted sequence of the cDNA of the invention. Usually multiple clones were sequenced from one or more human samples to derive the sequences for fragments. Various human tissue samples from different donors were used for the RACE reaction. The sequences derived from these procedures were included in the SeqCalling Assembly process described in preceding paragraphs.

5. Exon Linking: The NOVX target sequences identified in the present invention were subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The PCR product derived from exon linking was cloned into the pCR2.1 vector from Invitrogen. The resulting bacterial clone has an insert covering the entire open reading frame cloned into the pCR2.1 vector. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported herein.

6. Physical Clone: Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

The PCR product derived by exon linking, covering the entire open reading frame, was cloned into the pCR2.1 vector from Invitrogen to provide clones used for expression and screening purposes.

### Example C

### Quantitative Expression Analysis of Clones in Various Cells and Tissues

The quantitative expression of various NOV genes was assessed using microtiter plates containing RNA samples from a variety of normal and pathology-derived cells, cell lines and tissues using real time quantitative PCR (RTQ-PCR) performed on an Applied Biosystems (Foster City, Calif.) ABI PRISM® 7700 or an ABI PRISM® 7900 HT Sequence Detection System.

RNA integrity of all samples was determined by visual assessment of agarose gel electropherograms using 28S and 18S ribosomal RNA staining intensity ratio as a guide (2:1 to 2.5:1 28s:18s) and the absence of low molecular weight RNAs (degradation products). Control samples to detect genomic DNA contamination included RTQ-PCR reactions run in the absence of reverse transcriptase using probe and primer sets designed to amplify across the span of a single exon.

RNA samples were normalized in reference to nucleic acids encoding constitutively expressed genes (i.e., β-actin and GAPDH). Alternatively, non-normalized RNA samples were converted to single strand cDNA (sscDNA) using Superscript II (Invitrogen Corporation, Carlsbad, Calif., Catalog No. 18064-147) and random hexamers according to the manufacturer's instructions. Reactions containing up to 10 μg of total RNA in a volume of 20 μl or were scaled up to contain 50 μg of total RNA in a volume of 100 μl and were incubated for 60 minutes at 42° C. sscDNA samples were then normalized in reference to nucleic acids as described above.

Probes and primers were designed according to Applied Biosystems Primer Express Software package (version I for Apple Computer's Macintosh Power PC) or a similar algorithm using the target sequence as input. Default reaction condition settings and the following parameters were set before selecting primers: 250 nM primer concentration; 58°-60° C. primer melting temperature (Tₘ) range; 59° C. primer optimal Tm; 2° C. maximum primer difference (if probe does not have 5′ G, probe Tₘ must be 10° C. greater than primer Tₘ; and 75 bp to 100 bp amplicon size. The selected probes and primers were synthesized by Synthegen (Houston, Tex.). Probes were double purified by HPLC to remove uncoupled dye and evaluated by mass spectroscopy to verify coupling of reporter and quencher dyes to the 5′ and 3′ ends of the probe, respectively. Their final concentrations were: 900 nM forward and reverse primers, and 200 nM probe.

Normalized RNA was spotted in individual wells of a 96 or 384-well PCR plate (Applied Biosystems, Foster City, Calif.). PCR cocktails included a single gene-specific probe and primers set or two multiplexed probe and primers sets. PCR reactions were done using TaqMan® One-Step RT-PCR Master Mix (Applied Biosystems, Catalog No. 4313803) following manufacturer's instructions. Reverse transcription was performed at 48° C. for 30 minutes followed by amplification/PCR cycles: 95° C. 10 min, then 40 cycles at 95° C. for 15 seconds, followed by 60° C. for 1 minute. Results were recorded as CT values (cycle at which a given sample crosses a threshold level of fluorescence) and plotted using a log scale, with the difference in RNA concentration between a given sample and the sample with the lowest CT value being represented as 2 to the power of delta CT. The percent relative expression was the reciprocal of the RNA difference multiplied by 100. CT values below 28 indicate high expression, between 28 and 32 indicate moderate expression, between 32 and 35 indicate low expression and above 35 reflect levels of expression that were too low to be measured reliably.

Normalized sscDNA was analyzed by RTQ-PCR using 1×TaqMan® Universal Master mix (Applied Biosystems; catalog No. 4324020), following the manufacturer's instructions. PCR amplification and analysis were done as described above.

Panels 1, 1.1, 1.2, and 1.3D

Panels 1, 1. 1, 1.2 and 1 .3D included 2 control wells (genomic DNA control and chemistry control) and 94 wells of cDNA samples from cultured cell lines and primary normal tissues. Cell lines were derived from carcinomas (ca) including: lung, small cell (s cell var), non small cell (non-s or non-sm); breast; melanoma; colon; prostate; glioma (glio), astrocytoma (astro) and neuroblastoma (neuro); squamous cell (squam); ovarian; liver; renal; gastric and pancreatic from the American Type Culture Collection (ATCC, Bethesda, Md.). Normal tissues were obtained from individual adults or fetuses and included: adult and fetal skeletal muscle, adult and fetal heart, adult and fetal kidney, adult and fetal liver, adult and fetal lung, brain, spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose. The following abbreviations are used in reporting the results: metastasis (met); pleural effusion (pl. eff or pl effusion) and * indicates established from metastasis.

General_Screening_Panel_v1.4, v1.5, v1.6 and v1.7

Panels 1.4, 1.5, 1.6 and 1.7 were as described for Panels 1, 1.1, 1.2 and 1.3D, above except that normal tissue samples were pooled from 2 to 5 different adults or fetuses.

Panels 2D, 2.2, 2.3, and 2.4

Panels 2D, 2.2, 2.3 and 2.4 included 2 control wells and 94 wells containing RNA or cDNA from human surgical specimens procured through the National Cancer Institute's Cooperative Human Tissue Network (CHTN) or the National Disease Research Initiative (NDRI), Ardais (Lexington, Mass.) or Clinomics BioSciences (Frederick, Md.). Tissues included human malignancies and in some cases matched adjacent normal tissue (NAT). Information regarding histopathological assessment of tumor differentiation grade as well as the clinical stage of the patient from which samples were obtained was generally available. Normal tissue RNA and cDNA samples were purchased from various commercial sources such as Clontech (Palo Alto, Calif.), Research Genetics and Invitrogen (Carlsbad, Calif.).

HASS Panel v1.0

The HASS Panel v1.0 included 93 cDNA samples and two controls including: 81 samples of cultured human cancer cell lines subjected to serum starvation, acidosis and anoxia according to established procedures for various lengths of time; 3 human primary cells; 9 malignant brain cancers (4 medulloblastomas and 5 glioblastomas); and 2 controls. Cancer cell lines (ATCC) were cultured using recommended conditions and included: breast, prostate, bladder, pancreatic and CNS. Primary human cells were obtained from Clonetics (Walkersville, Md.). Malignant brain samples were gifts from the Henry Ford Cancer Center.

ARDAIS Panel v1.0 and v1.1

The ARDAIS Panel v1.0 and v1.1 included 2 controls and 22 test samples including: human lung adenocarcinomas, lung squamous cell carcinomas, and in some cases matched adjacent normal tissues (NAT) obtained from Ardais (Lexington, Mass.). Unmatched malignant and non-malignant RNA samples from lungs with gross histopathological assessment of tumor differentiation grade and stage and clinical state of the patient were obtained from Ardais.

ARDAIS Prostate v1.0

ARDAIS Prostate v1.0 panel included 2 controls and 68 test samples of human prostate malignancies and in some cases matched adjacent normal tissues (NAT) obtained from Ardais (Lexington, Mass.). RNA from unmatched malignant and non-malignant prostate samples with gross histopathological assessment of tumor differentiation grade and stage and clinical state of the patient were also obtained from Ardais.

ARDAIS Kidney v1.0

ARDAIS Kidney v1.0 panel included 2 control wells and 44 test samples of human renal cell carcinoma and in some cases matched adjacent normal tissue (NAT) obtained from Ardais (Lexington, Mass.). RNA from unmatched renal cell carcinoma and normal tissue with gross histopathological assessment of tumor differentiation grade and stage and clinical state of the patient were also obtained from Ardais.

ARDAIS Breast v1.0

ARDAIS Breast v1.0 panel included 2 control wells and 71 test samples of human breast malignancies and in some cases matched adjacent normal tissue (NAT) obtained from Ardais (Lexington, Mass.). RNA from unmatched malignant and non-malignant breast samples with gross histopathological assessment of tumor differentiation grade and stage and clinical state of the patient were also obtained from Ardais.

Panels 3D, 3.1 and 3.2

Panels 3D, 3.1, and 3.2 included two controls, 92 cDNA samples of cultured human cancer cell lines and 2 samples of human primary cerebellum. Cell lines (ATCC, National Cancer Institute (NCI), German tumor cell bank) were cultured as recommended and were derived from: squamous cell carcinoma of the tongue, melanoma, sarcoma, leukemia, lymphoma, and epidermoid, bladder, pancreas, kidney, breast, prostate, ovary, uterus, cervix, stomach, colon, lung and CNS carcinomas.

Panels 4D, 4R, and 4.1D

Panels 4D, 4R, and 4.1D included 2 control wells and 94 test samples of RNA (Panel 4R) or cDNA (Panels 4D and 4.1D) from human cell lines or tissues related to inflammatory conditions. Controls included total RNA from normal tissues such as colon, lung (Stratagene, La Jolla, Calif.), thymus and kidney (Clontech, Palo Alto, Calif.). Total RNA from cirrhotic and lupus kidney was obtained from BioChain Institute, Inc., (Hayward, Calif.). Crohn's intestinal and ulcerative colitis samples were obtained from the National Disease Research Interchange (NDRI, Philadelphia, Pa.). Cells purchased from Clonetics (Walkersville, Md.) included: astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, and human umbilical vein endothelial. These primary cell types were activated by incubating with various cytokines (IL-1 beta ˜1-5 ng/ml, TNF alpha ˜5-10 ng/ml, IFN gamma ˜20-50 ng/ml, IL-4˜5-10 ng/ml, IL-9˜5-10 ng/ml, IL-13 5-10 ng/ml) or combinations of cytokines as indicated. Starved endothelial cells were cultured in the basal media (Clonetics, Walkersville, Md.) with 0.1% serum.

Mononuclear cells were prepared from blood donations using Ficoll. LAK cells were cultured in culture media [DMEM, 5% FCS (flyclone, Logan, Utah), 100 μM non essential amino acids (Gibco/Life Technologies, Rockville, Md.), 1 mM sodium pyruvate (Gibco), mercaptoethanol 5.5×10⁻⁵ M (Gibco), and 10 mM Hepes (Gibco)] and interleukin 2 for 4-6 days. Cells were activated with 10-20 ng/ml PMA and 1-2 μg/ml ionomycin, 5-10 ng/ml IL-12, 20-50 ng/ml IFN gamma or 5-10 ng/ml IL-18 for 6 hours. In some cases, mononuclear cells were cultured for 4-5 days in culture media with ˜5 μg/ml PHA (phytohemagglutinin) or PWM (pokeweed mitogen; Sigma-Aldrich Corp., St. Louis, Mo.). Samples were taken at 24, 48 and 72 hours for RNA preparation. MLR (mixed lymphocyte reaction) samples were obtained by taking blood from two donors, isolating the mononuclear cells using Ficoll and mixing them 1:1 at a final concentration of ˜2×10⁶ cells/ml in culture media. The MLR samples were taken at various time points from 1-7 days for RNA preparation.

Monocytes were isolated from mononuclear cells using CD14 Miltenyi Beads, +ve VS selection columns and a Vario Magnet (Miltenyi Biotec, Auburn, Calif.) according to the manufacturer's instructions. Monocytes were differentiated into dendritic cells by culturing in culture media with 50 ng/ml GMCSF and 5 ng/mI IL-4 for 5-7 days. Macrophages were prepared by culturing monocytes for 5-7 days in culture media with ˜50 ng/ml 10% type AB Human Serum (Life technologies, Rockville, Md.) or MCSF (Macrophage colony stimulating factor; R&D, Minneapolis, Minn.). Monocytes, macropbages and dendritic cells were stimulated for 6 or 12-14 hours with 100 ng/ml lipopolysaccharide (LPS). Dendritic cells were also stimulated with 10 μg/ml anti-CD40 monoclonal antibody (Pharmingen, San Diego, Calif.) for 6 or 12-14 hours.

CD4+ lymphocytes, CD8+ lymphocytes and NK cells were also isolated from mononuclear cells using CD4, CD8 and CD56 Miltenyi beads, positive VS selection columns and a Vario Magnet (Miltenyi Biotec, Auburn, Calif.) according to the manufacturer's instructions. CD45+RA and CD45+RO CD4+ lymphocytes were isolated by depleting mononuclear cells of CD8+, CD56+, CD14+ and CD19+ cells using CD8, CD56, CD14 and CD19 Miltenyi beads and positive selection. CD45RO Miltenyi beads were then used to separate the CD45+RO CD4+ lymphocytes from CD45+RA CD4+ lymphocytes. CD45+RA CD4+, CD45+RO CD4 + and CD8+ lymphocytes were cultured in culture media at 10⁶ cells/ml in culture plates precoated overnight with 0.5 μg/ml anti-CD28 (Pharmingen, San Diego, Calif.) and 3 μg/ml anti-CD3 (OKT3, ATCC) in PBS. After 6 and 24 hours, the cells were harvested for RNA preparation. To prepare chronically activated CD8+ lymphocytes, isolated CD8+lymphocytes were activated for 4 days on anti-CD28, anti-CD3 coated plates and then harvested and expanded in culture media with IL-2 (1 ng/ml). These CD8+ cells were activated again with plate bound anti-CD3 and anti-CD28 for 4 days and expanded as described above. RNA was isolated 6 and 24 hours after the second activation and after 4 days of the second expansion culture. Isolated NK cells were cultured in culture media with 1 ng/ml IL-2 for 4-6 days before RNA was prepared.

B cells were prepared from minced and sieved tonsil tissue (NDRI). Tonsil cells were pelleted and resupended at 10⁶ cells/ml in culture media. Cells were activated using 5 μg/ml PWM (Sigma-Aldrich Corp., St. Louis, Mo.) or ˜10 μg/ml anti-CD40 (Pharmingen, San Diego, Calif.) and 5-10 ng/ml IL-4. Cells were harvested for RNA preparation after 24, 48 and 72 hours.

To prepare primary and secondary Th1/Th2 and Tr1 cells, umbilical cord blood CD4+ lymphocytes (Poietic Systems, German Town, Md.) were cultured at 10⁵⁻10⁶ cells/ml in culture media with IL-2 (4 ng/ml) in 6-well Falcon plates (precoated overnight with 10 μg/ml anti-CD28 (Pharmingen) and 2 μg/ml anti-CD3 (OKT3; ATCC) then washed twice with PBS).

To stimulate Th1 phenotype differentiation, IL-12 (5 ng/ml) and anti-IL4 (1 μg/ml) were used; for Th2 phenotype differentiation, IL-4 (5 ng/ml) and anti-IFN gamma (1 μg/ml) were used; and for Tr1 phenotype differentiation, IL-10 (5 ng/ml) was used. After 4-5 days, the activated Th1, Th2 and Tr1 lymphocytes were washed once with DMEM and expanded for 4-7 days in culture media with IL-2 (1 ng/ml). Activated Th1, Th2 and Tr1 lymphocytes were re-stimulated for 5 days with anti-CD28/CD3 and cytokines as described above with the addition of anti-CD95L (1 μg/ml) to prevent apoptosis. After 4-5 days, the Th1, Th2 and Tr1 lymphocytes were washed and expanded in culture media with IL-2 for 4-7 days. Activated Th1 and Th2 lymphocytes were maintained for a maximum of three cycles. RNA was prepared from primary and secondary Th1, Th2 and Tr1 after 6 and 24 hours following the second and third activations with plate-bound anti-CD3 and anti-CD28 mAbs and 4 days into the second and third expansion cultures.

Leukocyte cells lines Ramos, EOL-1, KU-812 were obtained from the ATCC. EOL-1 cells were further differentiated by culturing in culture media at 5×10⁵ cells/ml with 0.1 mM dbcAMP for 8 days, changing the media every 3 days and adjusting the cell concentration to 5×10⁵ cells/ml. RNA was prepared from resting cells or cells activated with PMA (10 ng/ml) and ionomycin (1 μg/ml) for 6 and 14 hours. RNA was prepared from resting CCD 1106 keratinocyte cell line (ATCC) or from cells activated with 5 ng/ml TNF alpha and 1 ng/ml IL-1 beta. RNA was prepared from resting NCI-H292, airway epithelial tumor cell line (ATCC) or from cells activated for 6 and 14 hours in culture media with 5 ng/ml IL-4, 5 ng/ml IL-9, 5 ng/ml IL-13, and 25 ng/ml IFN gamma.

RNA was prepared by lysing approximately 10⁷ cells/ml using Trizol (Gibco BRL) then adding 1/10 volume of bromochloropropane (Molecular Research Corporation, Cincinnati, Ohio), vortexing, incubating for 10 minutes at room temperature and then spinning at 14,000 rpm in a Sorvall SS34 rotor. The aqueous phase was placed in a 15 ml Falcon Tube and an equal volume of isopropanol was added and left at −20° C. overnight. The precipitated RNA was spun down at 9,000 rpm for 15 min and washed in 70% ethanol. The pellet was redissolved in 300 μl of RNAse-free water with 35 μl buffer (Promega, Madison, Wis.) 5 μl DTT, 7 μl RNA sin and 8 μl DNAse and incubated at 37° C. for 30 minutes to remove contaminating genomic DNA, extracted once with phenol chloroform and re-precipitated with 1/10 volume of 3 M sodium acetate and 2 volumes of 100% ethanol. The RNA was spun down, placed in RNAse free water and stored at −80° C.

AI Comprehensive Panel v1.0

Autoimmunity (AI) comprehensive panel v1.0 included two controls and 89 cDNA test samples isolated from male (M) and female (F) surgical and postmortem human tissues that were obtained from the Backus Hospital and Clinomics (Frederick, Md.). Tissue samples included: normal, adjacent (Adj); matched normal adjacent (match control); joint tissues (synovial (Syn) fluid, synovium, bone and cartilage, osteoarthritis (OA), rheumatoid arthritis (RA)); psoriatic; ulcerative colitis colon; Crohns disease colon; and emphysmatic, asthmatic, allergic and chronic obstructive pulmonary disease (COPD) lung.

Pulmonary and General Inflammation (PGI) Panel v1.0

Pulmonary and General inflammation (PGI) panel v1.0 included two controls and 39 test samples isolated as surgical or postmortem samples. Tissue samples include: five normal lung samples obtained from Maryland Brain and Tissue Bank, University of Maryland (Baltimore, Md.), International Bioresource systems, IBS (Tuscon, Ariz.), and Asterand (Detroit, Mich.), five normal adjacent intestine tissues (NAT) from Ardais (Lexington, Mass.), ulcerative colitis samples (UC) from Ardais (Lexington, Mass.); Crohns disease colon from NDRI, National Disease Research Interchange (Philadelphia, Pa.); emphysematous tissue samples from Ardais (Lexington, Mass.) and Genomic Collaborative Inc. (Cambridge, Mass.), asthmatic tissue from Maryland Brain and Tissue Bank, University of Maryland (Baltimore, Md.) and Genomic Collaborative Inc (Cambridge, Mass.) and fibrotic tissue from Ardais (Lexinton, Mass.) and Genomic Collaborative (Cambridge, Mass.).

AI.05 Chondrosarcoma

AI.05 chondrosarcoma plates included SW1353 cells (ATCC) subjected to serum starvation and treated for 6 and 18 h with cytokines that are known to induce MMP (1, 3 and 13) synthesis (e.g. IL 1beta). These treatments included: IL-1beta (10 ng/ml), IL-1beta +TNF-alpha (50 ng/ml), IL-1beta+Oncostatin (50 ng/ml) and PMA (100 ng/ml). Supernatants were collected and analyzed for MMP 1, 3 and 13 production. RNA was prepared from these samples using standard procedures.

Panels 5D and 5I

Panel 5D and 5I included two controls and cDNAs isolated from human tissues, human pancreatic islets cells, cell lines, metabolic tissues obtained from patients enrolled in the Gestational Diabetes study (described below), and cells from different stages of adipocyte differentiation, including differentiated (AD), midway differentiated (AM), and undifferentiated (U; human mesenchymal stem cells).

Gestational Diabetes study subjects were young (18-40 years), otherwise healthy women with and without gestational diabetes undergoing routine (elective) Caesarean section. Uterine wall smooth muscle (UT), visceral (Vis) adipose, skeletal muscle (SK), placenta (Pl) greater omentum adipose (GO Adipose) and subcutaneous (SubQ) adipose samples (<1 cc) were collected, rinsed in sterile saline, blotted and flash frozen in liquid nitrogen. Patients included: Patient 2, an overweight diabetic Hispanic not on insulin; Patient 7-9, obese non-diabetic Caucasians with body mass index (BMI) greater than 30; Patient 10, an overweight diabetic Hispanic, on insulin; Patient 11, an overweight nondiabetic African American; and Patient 12, a diabetic Hispanic on insulin.

Differentiated adipocytes were obtained from induced donor progenitor cells (Clonetics, Walkersville, Md.). Differentiated human mesenchymal stem cells (HuMSCs) were prepared as described in Mark F. Pittenger, et al., Multilineage Potential of Adult Human Mesenchymal Stem Cells Science Apr. 2, 1999: 143-147. mRNA was isolated and sscDNA was produced from Trizol lysates or frozen pellets. Human cell lines (ATCC, NCI or German tumor cell bank) included: kidney proximal convoluted tubule, uterine smooth muscle cells, small intestine, liver HepG2 cancer cells, heart primary stromal cells and adrenal cortical adenoma cells. Cells were cultured, RNA extracted and sscDNA was produced using standard procedures

Panel 5I also contains pancreatic islets (Diabetes Research Institute at the University of Miami School of Medicine).

Human Metabolic RTQ-PCR Panel

Human Metabolic RTQ-PCR Panel included two controls (genomic DNA control and chemistry control) and 211 cDNAs isolated from human tissues and cell lines relevant to metabolic diseases. This panel identifies genes that play a role in the etiology and pathogenesis of obesity and/or diabetes. Metabolic tissues including placenta (Pl), uterine wall smooth muscle (Ut), visceral adipose, skeletal muscle (Sk) and subcutaneous (SubQ) adipose were obtained from the Gestational Diabetes study (described above). Included in the panel are: Patients 7 and 8, obese non-diabetic Caucasians; Patient 12 a diabetic Caucasian with unknown BMI, on insulin (treated); Patient 13, an overweight diabetic Caucasian, not on insulin (untreated); Patient 15, an obese, untreated, diabetic Caucasian; Patient 17 and 25, untreated diabetic Caucasians of normal weight; Patient 18, an obese, untreated, diabetic Hispanic; Patient 19, a non-diabetic Caucasian of normal weight; Patient 20, an overweight, treated diabetic Caucasian; Patient 21 and 23, overweight non-diabetic Caucasians; Patient 22, a treated diabetic Caucasian of normal weight; Patient 23, an overweight non-diabetic Caucasian; and Patients 26 and 27, obese , treated, diabetic Caucasians.

Total RNA was isolated from metabolic tissues including: hypothalamus; liver, pancreas, pancreatic islets, small intestine, psoas muscle, diaphragm muscle, visceral (Vis) adipose, subcutaneous (SubQ) adipose and greater omentum (Go) from 12 Type II diabetic (Diab) patients and 12 non diabetic (Norm) at autopsy. Control diabetic and non-diabetic subjects were matched where possible for: age; sex, male (M); female (F); ethnicity, Caucasian (CC); Hispanic (HI); African American (AA); Asian (AS); and BMI, 20-25 (Low BM), 26-30 (Med BM) or overweight (Overwt), BMI greater than 30 (Hi BMI) (obese).

RNA was extracted and ss cDNA was produced from cell lines (ATCC) by standard methods.

CNS Panels

CNS Panels CNSD.01, CNS Neurodegeneration V1.0 and CNS Neurodegeneration V2.0 included two controls and 46 to 94 test cDNA samples isolated from postmortem human brain tissue obtained from the Harvard Brain Tissue Resource Center (McLean Hospital). Brains were removed from calvaria of donors between 4 and 24 hours after death, and frozen at −80° C. in liquid nitrogen vapor.

Panel CNSD.01

Panel CNSD.01 included two specimens each from: Alzheimer's disease, Parkinson's disease, Huntington's disease, Progressive Supernuclear Palsy (PSP), Depression, and normal controls. Collected tissues included: cingulate gyrus (Cing Gyr), temporal pole (Temp Pole), globus palladus (Glob palladus), substantia nigra (Sub Nigra), primary motor strip (Brodman Area 4), parietal cortex (Brodman Area 7), prefrontal cortex (Brodman Area 9), and occipital cortex (Brodman area 17). Not all brain regions are represented in all cases.

Panel CNS Neurodegeneration V1.0

The CNS Neurodegeneration V1.0 panel included: six Alzheimer's disease (AD) brains and eight normals which included no dementia and no Alzheimer's like pathology (control) or no dementia but evidence of severe Alzheimer's like pathology (Control Path), specifically senile plaque load rated as level 3 on a scale of 0-3; 0 no evidence of plaques, 3 severe AD senile plaque load. Tissues collected included: hippocampus, temporal cortex (Brodman Area 21), parietal cortex (Brodman area 7), occipital cortex (Brodman area 17) superior temporal cortex (Sup Temporal Ctx) and inferior temporal cortex (Inf Temproal Ctx).

Gene expression was analyzed after normalization using a scaling factor calculated by subtracting the Well mean (CT average for the specific tissue) from the Grand mean (average CT value for all wells across all runs). The scaled CT value is the result of the raw CT value plus the scaling factor.

Panel CNS Neurodegeneration V2.0

The CNS Neurodegeneration V2.0 panel included sixteen cases of Alzheimer's disease (AD) and twenty-nine normal controls (no evidence of dementia prior to death) including fourteen controls (Control) with no dementia and no Alzheimer's like pathology and fifteen controls with no dementia but evidence of severe Alzheimer's like pathology (AH3), specifically senile plaque load rated as level 3 on a scale of 0-3; 0 no evidence of plaques, 3 severe AD senile plaque load. Tissues from the temporal cortex (Brodman Area 21) included the inferior and superior temporal cortex that was pooled from a given individual (Inf & Sup Temp Ctx Pool).

A. CG103945-02: Semaphorin sem2.

Expression of gene CG103945-02 was assessed using the primer-probe set Ag7442, described in Table AA. Results of the RTQ-PCR runs are shown in Tables AB and AC.

TABLE AA

Probe Name Ag7442

| | |
|---|---|
| Start | SEQ |

| | | | | |
|---|---|---|---|---|
| Primers | Sequences | Length | Position | ID No |

| | | | | |
|---|---|---|---|---|
| Forward | 5′-gaaagccttccagcaccat-3′ | 19 | 128 | 283 |
| Probe | TET-5′-tggatggaaacattttccagatacctcc-3′-TAMRA | 28 | 148 | 284 |
| Reverse | 5′-gcccagaaagatggcagag-3′ | 19 | 189 | 285 |

TABLE AB
General screening panel v1.7

| Tissue Name | A |
|---|---|
| Adipose | 100.0 |
| HUVEC | 16.6 |
| Melanoma* Hs688(A).T | 0.0 |
| Melanoma* Hs688(B).T | 0.5 |
| Melanoma (met) SK-MEL-5 | 1.7 |
| Testis | 2.1 |
| Prostate ca. (bone met) PC-3 | 0.0 |
| Prostate ca. DU145 | 1.9 |
| Prostate pool | 1.5 |
| Uterus pool | 0.5 |
| Ovarian ca. OVCAR-3 | 0.0 |
| Ovarian ca. (ascites) SK-OV-3 | 0.0 |
| Ovarian ca. OVCAR-4 | 11.7 |
| Ovarian ca. OVCAR-5 | 7.2 |
| Ovarian ca. IGROV-1 | 2.9 |
| Ovarian ca. OVCAR-8 | 5.8 |
| Ovary | 13.6 |
| Breast ca. MCF-7 | 0.0 |
| Breast ca. MDA-MB-231 | 6.0 |
| Breast ca. BT-549 | 0.0 |
| Breast ca. T47D | 11.0 |
| Breast pool | 5.7 |
| Trachea | 13.0 |
| Lung | 66.0 |
| Fetal Lung | 12.3 |
| Lung ca. NCI-N417 | 0.0 |
| Lung ca. LX-1 | 0.0 |
| Lung ca. NCI-H146 | 0.0 |
| Lung ca. SHP-77 | 17.8 |
| Lung ca. NCI-H23 | 0.7 |
| Lung ca. NCI-H460 | 1.3 |
| Lung ca. HOP-62 | 0.0 |
| Lung ca. NCI-H522 | 4.4 |
| Lung ca. DMS-114 | 0.8 |
| Liver | 0.0 |
| Fetal Liver | 1.9 |
| Kidney pool | 28.7 |
| Fetal Kidney | 7.2 |
| Renal ca. 786-0 | 0.0 |
| Renal ca. A498 | 0.0 |
| Renal ca. ACHN | 0.0 |
| Renal ca. UO-31 | 0.0 |
| Renal ca. TK-10 | 0.0 |
| Bladder | 6.1 |
| Gastric ca. (liver met.) NCI-N87 | 0.0 |
| Stomach | 0.6 |
| Colon ca. SW-948 | 2.7 |
| Colon ca. SW480 | 0.0 |
| Colon ca. (SW480 met) SW620 | 5.1 |
| Colon ca. HT29 | 0.0 |
| Colon ca. HCT-116 | 19.1 |
| Colon cancer tissue | 0.9 |
| Colon ca. SW1116 | 5.9 |
| Colon ca. Colo-205 | 0.0 |
| Colon ca. SW-48 | 0.0 |
| Colon | 4.1 |
| Small Intestine | 0.8 |
| Fetal Heart | 2.0 |
| Heart | 1.3 |
| Lymph Node pool 1 | 2.1 |
| Lymph Node pool 2 | 20.7 |
| Fetal Skeletal Muscle | 6.8 |
| Skeletal Muscle pool | 1.1 |
| Skeletal Muscle | 9.7 |
| Spleen | 9.0 |
| Thymus | 1.1 |
| CNS cancer (glio/astro) SF-268 | 0.0 |
| CNS cancer (glio/astro) T98G | 0.0 |
| CNS cancer (neuro; met) SK-N-AS | 0.0 |
| CNS cancer (astro) SF-539 | 0.9 |
| CNS cancer (astro) SNB-75 | 0.0 |
| CNS cancer (glio) SNB-19 | 0.9 |
| CNS cancer (glio) SF-295 | 0.0 |
| Brain (Amygdala) | 3.1 |
| Brain (Cerebellum) | 22.4 |
| Brain (Fetal) | 6.2 |
| Brain (Hippocampus) | 4.5 |
| Cerebral Cortex pool | 5.1 |
| Brain (Substantia nigra) | 2.0 |
| Brain (Thalamus) | 4.1 |
| Brain (Whole) | 29.7 |
| Spinal Cord | 2.3 |
| Adrenal Gland | 2.8 |
| Pituitary Gland | 1.6 |
| Salivary Gland | 20.2 |
| Thyroid | 7.5 |
| Pancreatic ca. PANC-1 | 0.6 |
| Pancreas pool | 0.0 |

Column A - Rel. Exp. (%) Ag7442, Run 318350211

TABLE AC
Panel 4.1D

| Tissue Name | A |
|---|---|
| Secondary Th1 act | 0.0 |
| Secondary Th2 act | 4.4 |
| Secondary Tr1 act | 0.0 |
| Secondary Th1 rest | 0.0 |
| Secondary Th2 rest | 0.0 |
| Secondary Tr1 rest | 0.0 |
| Primary Th1 act | 0.0 |
| Primary Th2 act | 0.0 |
| Primary Tr1 act | 0.0 |
| Primary Th1 rest | 0.0 |
| Primary Th2 rest | 0.0 |
| Primary Tr1 rest | 0.0 |
| CD45RA CD4 lymphocyte act | 0.0 |
| CD45RO CD4 lymphocyte act | 0.0 |
| CD8 lymphocyte act | 0.0 |
| Secondary CD8 lymphocyte rest | 0.0 |
| Secondary CD8 lymphocyte act | 0.0 |
| CD4 lymphocyte none | 0.0 |
| 2ry Th1/Th2/Tr1_anti-CD95 CH11 | 0.0 |
| LAK cells rest | 0.0 |
| LAK cells IL-2 | 0.0 |
| LAK cells IL-2 + IL-12 | 0.0 |
| LAK cells IL-2 + IFN gamma | 0.0 |
| LAK cells IL-2 + IL-18 | 0.0 |
| LAK cells PMA/ionomycin | 0.0 |
| NK Cells IL-2 rest | 0.0 |
| Two Way MLR 3 day | 0.0 |
| Two Way MLR 5 day | 0.0 |
| Two Way MLR 7 day | 0.0 |
| PBMC rest | 0.0 |
| PBMC PWM | 0.0 |
| PBMC PHA-L | 0.0 |
| Ramos (B cell) none | 0.0 |
| Ramos (B cell) ionomycin | 0.0 |
| B lymphocytes PWM | 0.0 |
| B lymphocytes CD40L and IL-4 | 0.0 |
| EOL-1 dbcAMP | 6.1 |
| EOL-1 dbcAMP PMA/ionomycin | 4.2 |
| Dendritic cells none | 0.0 |
| Dendritic cells LPS | 3.6 |
| Dendritic cells anti-CD40 | 0.0 |
| Monocytes rest | 0.0 |
| Monocytes LPS | 0.0 |
| Macrophages rest | 0.0 |
| Macrophages LPS | 0.0 |
| HUVEC none | 0.0 |
| HUVEC starved | 11.0 |
| HUVEC IL-1beta | 4.7 |
| HUVEC IFN gamma | 0.0 |
| HUVEC TNF alpha + IFN gamma | 0.0 |
| HUVEC TNF alpha + IL4 | 2.6 |
| HUVEC IL-11 | 13.4 |
| Lung Microvascular EC none | 100.0 |
| Lung Microvascular EC TNFalpha + IL-1beta | 48.3 |
| Microvascular Dermal EC none | 30.4 |
| Microsvasular Dermal EC TNFalpha + IL-1beta | 19.9 |
| Bronchial epithelium TNFalpha + IL1beta | 0.0 |
| Small airway epithelium none | 0.0 |
| Small airway epithelium TNFalpha + IL-1beta | 0.0 |
| Coronery artery SMC rest | 0.0 |
| Coronery artery SMC TNFalpha + IL-1beta | 0.0 |
| Astrocytes rest | 0.0 |
| Astrocytes TNFalpha + IL-1beta | 0.0 |
| KU-812 (Basophil) rest | 0.0 |
| KU-812 (Basophil) PMA/ionomycin | 5.0 |
| CCD1106 (Keratinocytes) none | 0.0 |
| CCD1106 (Keratinocytes) TNFalpha + IL-1beta | 0.0 |
| Liver cirrhosis | 11.9 |
| NCI-H292 none | 0.0 |
| NCI-H292 IL-4 | 0.0 |
| NCI-H292 IL-9 | 0.0 |
| NCI-H292 IL-13 | 0.0 |
| NCI-H292 IFN gamma | 0.0 |
| HPAEC none | 4.7 |
| HPAEC TNF alpha + IL-1 beta | 5.2 |
| Lung fibroblast none | 0.0 |
| Lung fibroblast TNF alpha + IL-1 beta | 0.0 |
| Lung fibroblast IL-4 | 0.0 |
| Lung fibroblast IL-9 | 0.0 |
| Lung fibroblast IL-13 | 0.0 |
| Lung fibroblast IFN gamma | 0.0 |
| Dermal fibroblast CCD1070 rest | 0.0 |
| Dermal fibroblast CCD1070 TNF alpha | 0.0 |
| Dermal fibroblast CCD1070 IL-1 beta | 0.0 |
| Dermal fibroblast IFN gamma | 0.0 |
| Dermal fibroblast IL-4 | 0.0 |
| Dermal Fibroblasts rest | 5.6 |
| Neutrophils TNFa + LPS | 0.0 |
| Neutrophils rest | 0.0 |
| Colon | 14.5 |
| Lung | 0.0 |
| Thymus | 0.0 |
| Kidney | 6.0 |

Column A - Rel. Exp. (%) Ag7442, Run 306067441

General_screening_panel_v1.7 Summary: Ag7442 Highest expression of the CG103945-02 gene was detected in adipose tissue (CT=29.5). In addition, significant expression of this gene was also seen in skeletal muscle and thyroid. Therapeutic modulation of the activity of this gene or its protein product is useful in the treatment of metabolic disorders, including diabetes and obesity.

Moderate levels of expression of this gene were also seen in all regions of the central nervous system examined, including amygdala, hippocampus, substantia nigra, thalamus, cerebellum, cerebral cortex, and spinal cord. Therapeutic modulation of the activity of this gene or its protein product is useful in the treatment of central nervous system disorders such as Alzheimer's disease, Parkinson's disease, epilepsy, multiple sclerosis, schizophrenia and depression.

Moderate to low expression of this gene was seen in number of cancer cell lines derived from colon, lung, breast and ovarian cancers. Therapeutic modulation of the activity of this gene or its protein product is useful in the treatment of colon, lung, breast and ovarian cancers.

Panel 4.1D Summary: Ag7442 Highest expression of this gene was seen in lung microvascular endothelium (CT=33.6) and its expression was down-regulated upon activation of these cells. Endothelial cells are known to play important roles in inflammatory responses by altering the expression of surface proteins that are involved in activation and recruitment of effector inflammatory cells. Higher expression of this gene in resting cells suggests a role for this gene in the maintenance of the integrity of the lung microvasculature. Therapeutic modulation of the activity of this gene or its protein product is beneficial for the treatment of diseases associated with damaged microvasculature including heart diseases or inflammatory diseases, such as psoriasis, asthma, and chronic obstructive pulmonary diseases.

B. CG106951-01 and CG106951-04: Semaphorin 5B.

Expression of gene CG106951-01 and CG106951-04 was assessed using the primer-probe sets Ag1216, described in Tables BA.

TABLE BA
Probe Name Ag1216

| | | | Start | SEQ ID |
|---|---|---|---|---|
| Primers | Sequences | Length | Position | No |
| Forward | 5′-cccgaagaatgaaaagta | 22 | 3351 | 286 |
| | caca-3′ | | | |
| Probe | TET-5′-cccatggaattcaa | 26 | 3373 | 287 |
| | gaccctgaacaa-3′-TAMRA | | | |
| Reverse | 5′-aatgggtagaagttggct | 22 | 3419 | 288 |
| | ctgt-3′ | | | |

TABLE BB
AI comprehensive panel v1.0

| Tissue Name | A |
|---|---|
| 110967 COPD-F | 10.4 |
| 110980 COPD-F | 23.5 |
| 110968 COPD-M | 13.6 |
| 110977 COPD-M | 64.6 |
| 110989 Emphysema-F | 35.4 |
| 110992 Emphysema-F | 13.5 |
| 110993 Emphysema-F | 27.4 |
| 110994 Emphysema-F | 5.6 |
| 110995 Emphysema-F | 15.3 |
| 110996 Emphysema-F | 7.2 |
| 110997 Asthma-M | 9.2 |
| 111001 Asthma-F | 24.0 |
| 111002 Asthma-F | 26.6 |
| 111003 Atopic Asthma-F | 35.1 |
| 111004 Atopic Asthma-F | 24.1 |
| 111005 Atopic Asthma-F | 14.0 |
| 111006 Atopic Asthma-F | 3.2 |
| 111417 Allergy-M | 18.0 |
| 112347 Allergy-M | 0.5 |
| 112349 Normal Lung-F | 0.5 |
| 112357 Normal Lung-F | 56.6 |
| 112354 Normal Lung-M | 14.0 |
| 112374 Crohns-F | 42.3 |
| 112389 Match Control Crohns-F | 61.6 |
| 112375 Crohns-F | 25.7 |
| 112732 Match Control Crohns-F | 7.5 |
| 112725 Crohns-M | 3.1 |
| 112387 Match Control Crohns-M | 60.7 |
| 112378 Crohns-M | 0.8 |
| 112390 Match Control Crohns-M | 24.5 |
| 112726 Crohns-M | 24.1 |
| 112731 Match Control Crohns-M | 12.4 |
| 112380 Ulcer Col-F | 18.6 |
| 112734 Match Control Ulcer Col-F | 30.6 |
| 112384 Ulcer Col-F | 45.7 |
| 112737 Match Control Ulcer Col-F | 16.5 |
| 112386 Ulcer Col-F | 13.8 |
| 112738 Match Control Ulcer Col-F | 3.0 |
| 112381 Ulcer Col-M | 1.5 |
| 112735 Match Control Ulcer Col-M | 8.8 |
| 112382 Ulcer Col-M | 25.7 |
| 112394 Match Control Ulcer Col-M | 21.2 |
| 112383 Ulcer Col-M | 42.6 |
| 112736 Match Control Ulcer Col-M | 22.8 |
| 112423 Psoriasis-F | 43.8 |
| 112427 Match Control Psoriasis-F | 29.7 |
| 112418 Psoriasis-M | 8.4 |
| 112723 Match Control Psoriasis-M | 2.2 |
| 112419 Psoriasis-M | 16.3 |
| 112424 Match Control Psoriasis-M | 24.0 |
| 112420 Psoriasis-M | 41.5 |
| 112425 Match Control Psoriasis-M | 43.5 |
| 104689 (MF) OA Bone-Backus | 35.6 |
| 104690 (MF) Adj “Normal” Bone-Backus | 12.5 |
| 104691 (MF) OA Synovium-Backus | 14.9 |
| 104692 (BA) OA Cartilage-Backus | 0.0 |
| 104694 (BA) OA Bone-Backus | 10.5 |
| 104695 (BA) Adj “Normal” Bone-Backus | 5.7 |
| 104696 (BA) OA Synovium-Backus | 6.7 |
| 104700 (SS) OA Bone-Backus | 6.0 |
| 104701 (SS) Adj “Normal” Bone-Backus | 5.8 |
| 104702 (SS) OA Synovium-Backus | 48.3 |
| 117093 OA Cartilage Rep7 | 23.5 |
| 112672 OA Bone5 | 26.1 |
| 112673 OA Synovium5 | 14.0 |
| 112674 OA Synovial Fluid cells5 | 12.6 |
| 117100 OA Cartilage Rep14 | 6.7 |
| 112756 OA Bone9 | 100.0 |
| 112757 OA Synovium9 | 2.1 |
| 112758 OA Synovial Fluid Cells9 | 28.9 |
| 117125 RA Cartilage Rep2 | 5.2 |
| 113492 Bone2 RA | 5.0 |
| 113493 Synovium2 RA | 16.2 |
| 113494 Syn Fluid Cells RA | 16.5 |
| 113499 Cartilage4 RA | 13.2 |
| 113500 Bone4 RA | 9.2 |
| 113501 Synovium4 RA | 12.6 |
| 113502 Syn Fluid Cells4 RA | 8.9 |
| 113495 Cartilage3 RA | 5.6 |
| 113496 Bone3 RA | 8.3 |
| 113497 Synovium3 RA | 4.7 |
| 113498 Syn Fluid Cells3 RA | 5.1 |
| 117106 Normal Cartilage Rep20 | 10.7 |
| 113663 Bone3 Normal | 1.1 |
| 113664 Synovium3 Normal | 0.0 |
| 113665 Syn Fluid Cells3 Normal | 0.9 |
| 117107 Normal Cartilage Rep22 | 12.6 |
| 113667 Bone4 Normal | 4.3 |
| 113668 Synovium4 Normal | 10.4 |
| 113669 Syn Fluid Cells4 Normal | 7.0 |

Column A - Rel. Exp. (%) Ag1216, Run 233667803

TABLE BC
General screening panel v1.4

| Tissue Name | A |
|---|---|
| Adipose | 1.3 |
| Melanoma* Hs688(A).T | 0.0 |
| Melanoma* Hs688(B).T | 0.0 |
| Melanoma* M14 | 0.0 |
| Melanoma* LOXIMVI | 0.0 |
| Melanoma* SK-MEL-5 | 0.0 |
| Squamous cell carcinoma SCC-4 | 0.1 |
| Testis Pool | 0.4 |
| Prostate ca.* (bone met) PC-3 | 0.0 |
| Prostate Pool | 1.0 |
| Placenta | 1.8 |
| Uterus Pool | 0.8 |
| Ovarian ca. OVCAR-3 | 18.8 |
| Ovarian ca. SK-OV-3 | 0.0 |
| Ovarian ca. OVCAR-4 | 0.0 |
| Ovarian ca. OVCAR-5 | 0.6 |
| Ovarian ca. IGROV-1 | 0.5 |
| Ovarian ca. OVCAR-8 | 0.3 |
| Ovary | 1.8 |
| Breast ca. MCF-7 | 0.1 |
| Breast ca. MDA-MB-231 | 0.0 |
| Breast ca. BT 549 | 15.2 |
| Breast ca. T47D | 1.4 |
| Breast ca. MDA-N | 0.0 |
| Breast Pool | 2.1 |
| Trachea | 0.7 |
| Lung | 0.3 |
| Fetal Lung | 10.3 |
| Lung ca. NCI-N417 | 0.0 |
| Lung ca. LX-1 | 0.0 |
| Lung ca. NCI-H146 | 0.2 |
| Lung ca. SHP-77 | 0.0 |
| Lung ca. A549 | 0.0 |
| Lung ca. NCI-H526 | 0.0 |
| Lung ca. NCI-H23 | 0.2 |
| Lung ca. NCI-H460 | 0.0 |
| Lung ca. HOP-62 | 0.0 |
| Lung ca. NCI-H522 | 0.2 |
| Liver | 0.1 |
| Fetal Liver | 1.9 |
| Liver ca. HepG2 | 0.0 |
| Kidney Pool | 0.8 |
| Fetal Kidney | 16.6 |
| Renal ca. 786-0 | 100.0 |
| Renal ca. A498 | 3.6 |
| Renal ca. ACHN | 0.1 |
| Renal ca. UO-31 | 0.1 |
| Renal ca. TK-10 | 0.1 |
| Bladder | 1.0 |
| Gastric ca. (liver met.) NCI-N87 | 0.1 |
| Gastric ca. KATO III | 0.0 |
| Colon ca. SW-948 | 0.0 |
| Colon ca. SW480 | 0.0 |
| Colon ca.* (SW480 met) SW620 | 0.2 |
| Colon ca. HT29 | 0.0 |
| Colon ca. HCT-116 | 0.1 |
| Colon ca. CaCo-2 | 0.4 |
| Colon cancer tissue | 0.5 |
| Colon ca. SW1116 | 0.0 |
| Colon ca. Colo-205 | 0.0 |
| Colon ca. SW-48 | 0.0 |
| Colon Pool | 2.0 |
| Small Intestine Pool | 1.0 |
| Stomach Pool | 1.0 |
| Bone Marrow Pool | 1.0 |
| Fetal Heart | 12.9 |
| Heart Pool | 0.9 |
| Lymph Node Pool | 1.9 |
| Fetal Skeletal Muscle | 12.8 |
| Skeletal Muscle Pool | 0.4 |
| Spleen Pool | 0.1 |
| Thymus Pool | 1.0 |
| CNS cancer (glio/astro) U87-MG | 0.0 |
| CNS cancer (glio/astro) U-118-MG | 2.0 |
| CNS cancer (neuro; met) SK-N-AS | 2.1 |
| CNS cancer (astro) SF-539 | 0.1 |
| CNS cancer (astro) SNB-75 | 5.4 |
| CNS cancer (glio) SNB-19 | 0.3 |
| CNS cancer (glio) SF-295 | 0.0 |
| Brain (Amygdala) Pool | 3.6 |
| Brain (cerebellum) | 3.7 |
| Brain (fetal) | 30.1 |
| Brain (Hippocampus) Pool | 3.6 |
| Cerebral Cortex Pool | 6.5 |
| Brain (Substantia nigra) Pool | 4.6 |
| Brain (Thalamus) Pool | 6.0 |
| Brain (whole) | 13.0 |
| Spinal Cord Pool | 1.2 |
| Adrenal Gland | 2.0 |
| Pituitary gland Pool | 0.5 |
| Salivary Gland | 0.1 |
| Thyroid (female) | 0.2 |
| Pancreatic ca. CAPAN2 | 0.0 |
| Pancreas Pool | 2.4 |

Column A - Rel. Exp. (%) Ag1216, Run 212696280

TABLE BD
Panel 3D

| Tissue Name | A |
|---|---|
| Daoy- Medulloblastoma | 0.9 |
| TE671- Medulloblastoma | 0.0 |
| D283 Med- Medulloblastoma | 2.8 |
| PFSK-1- Primitive Neuroectodermal | 0.9 |
| XF-498- CNS | 0.0 |
| SNB-78- Glioma | 3.7 |
| SF-268- Glioblastoma | 0.0 |
| T98G- Glioblastoma | 0.0 |
| SK-N-SH- Neuroblastoma (metastasis) | 2.7 |
| SF-295- Glioblastoma | 0.0 |
| Cerebellum | 1.3 |
| Cerebellum | 5.0 |
| NCI-H292- Mucoepidermoid lung carcinoma | 1.7 |
| DMS-114- Small cell lung cancer | 0.8 |
| DMS-79- Small cell lung cancer | 100.0 |
| NCI-H146- Small cell lung cancer | 3.3 |
| NCI-H526- Small cell lung cancer | 0.0 |
| NCI-N417- Small cell lung cancer | 0.0 |
| NCI-H82- Small cell lung cancer | 3.6 |
| NCI-H157- Squamous cell lung cancer (metastasis) | 0.0 |
| NCI-H1155- Large cell lung cancer | 0.0 |
| NCI-H1299- Large cell lung cancer | 0.0 |
| NCI-H727- Lung carcinoid | 0.0 |
| NCI-UMC-11- Lung carcinoid | 0.0 |
| LX-1- Small cell lung cancer | 0.0 |
| Colo-205- Colon cancer | 0.0 |
| KM12- Colon cancer | 0.0 |
| KM20L2- Colon cancer | 0.0 |
| NCI-H716- Colon cancer | 0.0 |
| SW-48- Colon adenocarcinoma | 0.0 |
| SW1116- Colon adenocarcinoma | 0.0 |
| LS 174T- Colon adenocarcinoma | 0.0 |
| SW-948- Colon adenocarcinoma | 0.0 |
| SW-480- Colon adenocarcinoma | 0.0 |
| NCI-SNU-5- Gastric carcinoma | 0.0 |
| KATO III- Gastric carcinoma | 0.0 |
| NCI-SNU-16- Gastric carcinoma | 0.0 |
| NCI-SNU-1- Gastric carcinoma | 0.0 |
| RF-1- Gastric adenocarcinoma | 0.0 |
| RF-48- Gastric adenocarcinoma | 0.3 |
| MKN-45- Gastric carcinoma | 0.0 |
| NCI-N87- Gastric carcinoma | 0.6 |
| OVCAR-5- Ovarian carcinoma | 0.0 |
| RL95-2- Uterine carcinoma | 0.0 |
| HelaS3- Cervical adenocarcinoma | 0.0 |
| Ca Ski- Cervical epidermoid carcinoma (metastasis) | 0.0 |
| ES-2- Ovarian clear cell carcinoma | 0.0 |
| Ramos- Stimulated with PMA/ionomycin 6 h | 0.0 |
| Ramos- Stimulated with PMA/ionomycin 14 h | 0.0 |
| MEG-01- Chronic myelogenous leukemia | 0.0 |
| (megokaryoblast) | |
| Raji- Burkitt's lymphoma | 1.4 |
| Daudi- Burkitt's lymphoma | 0.0 |
| U266- B-cell plasmacytoma | 0.0 |
| CA46- Burkitt's lymphoma | 0.0 |
| RL- non-Hodgkin's B-cell lymphoma | 0.0 |
| JM1- pre-B-cell lymphoma | 0.0 |
| Jurkat- T cell leukemia | 0.0 |
| TF-1- Erythroleukemia | 0.0 |
| HUT 78- T-cell lymphoma | 0.0 |
| U937- Histiocytic lymphoma | 0.0 |
| KU-812- Myelogenous leukemia | 0.0 |
| 769-P- Clear cell renal carcinoma | 1.9 |
| Caki-2- Clear cell renal carcinoma | 2.1 |
| SW 839- Clear cell renal carcinoma | 8.8 |
| Rhabdoid kidney tumor | 0.2 |
| Hs766T- Pancreatic carcinoma (LN metastasis) | 0.0 |
| CAPAN-1- Pancreatic adenocarcinoma (liver | 0.0 |
| metastasis) | |
| SU86.86- Pancreatic carcinoma (liver metastasis) | 2.8 |
| BxPC-3- Pancreatic adenocarcinoma | 0.0 |
| HPAC- Pancreatic adenocarcinoma | 0.0 |
| MIA PaCa-2- Pancreatic carcinoma | 0.0 |
| CFPAC-1- Pancreatic ductal adenocarcinoma | 0.0 |
| PANC-1- Pancreatic epithelioid ductal carcinoma | 1.7 |
| T24- Bladder carcinma (transitional cell) | 0.0 |
| 5637- Bladder carcinoma | 2.0 |
| HT-1197- Bladder carcinoma | 0.0 |
| UM-UC-3- Bladder carcinma (transitional cell) | 0.6 |
| A204- Rhabdomyosarcoma | 0.0 |
| HT-1080- Fibrosarcoma | 0.0 |
| MG-63- Osteosarcoma | 0.0 |
| SK-LMS-1- Leiomyosarcoma (vulva) | 0.0 |
| SJRH30- Rhabdomyosarcoma (met to bone marrow) | 0.0 |
| A431- Epidermoid carcinoma | 0.0 |
| WM266-4- Melanoma | 0.0 |
| DU 145- Prostate carcinoma (brain metastasis) | 0.0 |
| MDA-MB-468- Breast adenocarcinoma | 0.8 |
| SCC-4- Squamous cell carcinoma of tongue | 0.0 |
| SCC-9- Squamous cell carcinoma of tongue | 0.8 |
| SCC-15- Squamous cell carcinoma of tongue | 0.0 |
| CAL 27- Squamous cell carcinoma of tongue | 0.0 |

Column A - Rel. Exp. (%) Ag1216, Run 182098855

TABLE BE
Panel 4D

| Tissue Name | A | B |
|---|---|---|
| Secondary Th1 act | 0.0 | 0.0 |
| Secondary Th2 act | 0.0 | 0.0 |
| Secondary Tr1 act | 0.0 | 0.0 |
| Secondary Th1 rest | 0.0 | 0.0 |
| Secondary Th2 rest | 0.0 | 0.0 |
| Secondary Tr1 rest | 0.0 | 0.0 |
| Primary Th1 act | 0.0 | 0.0 |
| Primary Th2 act | 0.0 | 0.0 |
| Primary Tr1 act | 0.0 | 0.0 |
| Primary Th1 rest | 0.0 | 0.0 |
| Primary Th2 rest | 1.1 | 0.0 |
| Primary Tr1 rest | 0.0 | 0.9 |
| CD45RA CD4 lymphocyte act | 0.0 | 0.0 |
| CD45RO CD4 lymphocyte act | 0.8 | 0.0 |
| CD8 lymphocyte act | 0.0 | 0.0 |
| Secondary CD8 lymphocyte rest | 0.0 | 1.3 |
| Secondary CD8 lymphocyte act | 0.0 | 0.0 |
| CD4 lymphocyte none | 0.0 | 0.9 |
| 2ry Th1/Th2/Tr1_anti-CD95 CH11 | 0.0 | 0.0 |
| LAK cells rest | 0.0 | 0.0 |
| LAK cells IL-2 | 0.0 | 0.0 |
| LAK cells IL-2 + IL-12 | 0.0 | 1.9 |
| LAK cells IL-2 + IFN gamma | 1.1 | 0.0 |
| LAK cells IL-2 + IL-18 | 0.0 | 0.0 |
| LAK cells PMA/ionomycin | 0.0 | 0.0 |
| NK Cells IL-2 rest | 0.0 | 0.0 |
| Two Way MLR 3 day | 0.0 | 0.0 |
| Two Way MLR 5 day | 0.0 | 0.0 |
| Two Way MLR 7 day | 0.0 | 0.0 |
| PBMC rest | 0.0 | 0.0 |
| PBMC PWM | 2.8 | 2.5 |
| PBMC PHA-L | 0.0 | 3.1 |
| Ramos (B cell) none | 0.0 | 0.0 |
| Ramos (B cell) ionomycin | 0.0 | 0.0 |
| B lymphocytes PWM | 0.0 | 0.0 |
| B lymphocytes CD40L and IL-4 | 1.1 | 0.0 |
| EOL-1 dbcAMP | 0.0 | 0.0 |
| EOL-1 dbcAMP PMA/ionomycin | 0.0 | 0.0 |
| Dendritic cells none | 0.0 | 0.0 |
| Dendritic cells LPS | 0.0 | 0.0 |
| Dendritic cells anti-CD40 | 0.0 | 0.0 |
| Monocytes rest | 0.0 | 0.0 |
| Monocytes LPS | 0.0 | 0.0 |
| Macrophages rest | 0.0 | 0.0 |
| Macrophages LPS | 0.8 | 0.0 |
| HUVEC none | 0.0 | 0.0 |
| HUVEC starved | 0.0 | 0.0 |
| HUVEC IL-1beta | 0.0 | 1.3 |
| HUVEC IFN gamma | 0.0 | 0.0 |
| HUVEC TNF alpha + IFN gamma | 0.0 | 0.0 |
| HUVEC TNF alpha + IL4 | 0.0 | 0.0 |
| HUVEC IL-11 | 0.9 | 0.0 |
| Lung Microvascular EC none | 0.0 | 0.0 |
| Lung Microvascular EC TNFalpha + IL-1beta | 0.0 | 0.0 |
| Microvascular Dermal EC none | 0.0 | 0.0 |
| Microsvasular Dermal EC TNFalpha + IL-1beta | 0.0 | 0.0 |
| Bronchial epithelium TNFalpha + IL1beta | 20.3 | 31.9 |
| Small airway epithelium none | 1.8 | 2.0 |
| Small airway epithelium TNFalpha + IL-1beta | 2.7 | 2.5 |
| Coronery artery SMC rest | 0.0 | 0.0 |
| Coronery artery SMC TNFalpha + IL-1beta | 0.0 | 0.0 |
| Astrocytes rest | 12.8 | 16.3 |
| Astrocytes TNFalpha + IL-1beta | 5.3 | 13.5 |
| KU-812 (Basophil) rest | 0.0 | 0.0 |
| KU-812 (Basophil) PMA/ionomycin | 0.0 | 0.0 |
| CCD1106 (Keratinocytes) none | 1.4 | 0.0 |
| CCD1106 (Keratinocytes) TNFalpha + IL-1beta | 0.0 | 4.1 |
| Liver cirrhosis | 1.9 | 2.3 |
| Lupus kidney | 5.9 | 10.3 |
| NCI-H292 none | 1.6 | 0.0 |
| NCI-H292 IL-4 | 0.7 | 0.0 |
| NCI-H292 IL-9 | 0.0 | 2.8 |
| NCI-H292 IL-13 | 0.0 | 0.0 |
| NCI-H292 IFN gamma | 0.0 | 0.0 |
| HPAEC none | 0.0 | 0.0 |
| HPAEC TNF alpha + IL-1 beta | 0.0 | 0.0 |
| Lung fibroblast none | 0.0 | 0.0 |
| Lung fibroblast TNF alpha + IL-1 beta | 0.0 | 0.0 |
| Lung fibroblast IL-4 | 0.0 | 0.0 |
| Lung fibroblast IL-9 | 0.0 | 0.0 |
| Lung fibroblast IL-13 | 0.0 | 0.0 |
| Lung fibroblast IFN gamma | 0.0 | 0.0 |
| Dermal fibroblast CCD1070 rest | 0.0 | 0.0 |
| Dermal fibroblast CCD1070 TNF alpha | 0.0 | 0.0 |
| Dermal fibroblast CCD1070 IL-1 beta | 0.0 | 0.0 |
| Dermal fibroblast IFN gamma | 0.0 | 0.0 |
| Dermal fibroblast IL-4 | 0.0 | 1.5 |
| IBD Colitis 2 | 0.0 | 1.2 |
| IBD Crohn's | 1.4 | 4.0 |
| Colon | 3.4 | 2.7 |
| Lung | 52.1 | 42.3 |
| Thymus | 100.0 | 100.0 |
| Kidney | 1.6 | 0.0 |

Column A - Rel. Exp. (%) Ag1216, Run 140426332
Column B - Rel. Exp. (%) Ag1216, Run 144134834

TABLE BF
Panel 5 Islet

| Tissue Name | A |
|---|---|
| 97457_Patient-02go_adipose | 41.8 |
| 97476_Patient-07sk_skeletal muscle | 36.3 |
| 97477_Patient-07ut_uterus | 34.9 |
| 97478_Patient-07pl_placenta | 100.0 |
| 99167_Bayer Patient 1 | 0.0 |
| 97482_Patient-08ut_uterus | 23.5 |
| 97483_Patient-08pl_placenta | 35.1 |
| 97486_Patient-09sk_skeletal muscle | 9.1 |
| 97487_Patient-09ut_uterus | 81.2 |
| 97488_Patient-09pl_placenta | 26.6 |
| 97492_Patient-10ut_uterus | 75.3 |
| 97493_Patient-10pl_placenta | 50.0 |
| 97495_Patient-11go_adipose | 57.8 |
| 97496_Patient-11sk_skeletal muscle | 34.2 |
| 97497_Patient-11ut_uterus | 89.5 |
| 97498_Patient-11pl_placenta | 77.9 |
| 97500_Patient-12go_adipose | 70.7 |
| 97501_Patient-12sk_skeletal muscle | 43.2 |
| 97502_Patient-12ut_uterus | 97.3 |
| 97503_Patient-12pl_placenta | 24.5 |
| 94721_Donor 2 U - A_Mesenchymal Stem Cells | 0.0 |
| 94722_Donor 2 U - B_Mesenchymal Stem Cells | 0.0 |
| 94723_Donor 2 U - C_Mesenchymal Stem Cells | 0.0 |
| 94709_Donor 2 AM - A_adipose | 0.0 |
| 94710_Donor 2 AM - B_adipose | 0.0 |
| 94711_Donor 2 AM - C_adipose | 0.0 |
| 94712_Donor 2 AD - A_adipose | 0.0 |
| 94713_Donor 2 AD - B_adipose | 0.0 |
| 94714_Donor 2 AD - C_adipose | 0.0 |
| 94742_Donor 3 U - A_Mesenchymal Stem Cells | 0.0 |
| 94743_Donor 3 U - B_Mesenchymal Stem Cells | 0.0 |
| 94730_Donor 3 AM - A_adipose | 0.0 |
| 94731_Donor 3 AM - B_adipose | 0.0 |
| 94732_Donor 3 AM - C_adipose | 0.0 |
| 94733_Donor 3 AD - A_adipose | 0.0 |
| 94734_Donor 3 AD - B_adipose | 0.0 |
| 94735_Donor 3 AD - C_adipose | 0.0 |
| 77138_Liver_HepG2untreated | 0.0 |
| 73556_Heart_Cardiac stromal cells (primary) | 0.0 |
| 81735_Small Intestine | 29.3 |
| 72409_Kidney_Proximal Convoluted Tubule | 0.0 |
| 82685_Small intestine_Duodenum | 6.9 |
| 90650_Adrenal_Adrenocortical adenoma | 17.7 |
| 72410_Kidney_HRCE | 5.5 |
| 72411_Kidney_HRE | 4.9 |
| 73139_Uterus_Uterine smooth muscle cells | 0.0 |

Column A - Rel. Exp. (%) Ag1216, Run 237228676

TABLE BG
general oncology screening panel v 2.4

| Tissue Name | A |
|---|---|
| Colon cancer 1 | 0.9 |
| Colon cancer NAT 1 | 0.5 |
| Colon cancer 2 | 0.2 |
| Colon cancer NAT 2 | 0.3 |
| Colon cancer 3 | 0.7 |
| Colon cancer NAT 3 | 0.4 |
| Colon malignant cancer 4 | 1.3 |
| Colon normal adjacent tissue 4 | 0.2 |
| Lung cancer 1 | 0.7 |
| Lung NAT 1 | 0.1 |
| Lung cancer 2 | 10.8 |
| Lung NAT 2 | 0.1 |
| Squamous cell carcinoma 3 | 2.0 |
| Lung NAT 3 | 0.0 |
| metastatic melanoma 1 | 1.4 |
| Melanoma 2 | 0.0 |
| Melanoma 3 | 0.5 |
| metastatic melanoma 4 | 2.7 |
| metastatic melanoma 5 | 8.0 |
| Bladder cancer 1 | 0.2 |
| Bladder cancer NAT 1 | 0.0 |
| Bladder cancer 2 | 0.3 |
| Bladder cancer NAT 2 | 0.1 |
| Bladder cancer NAT 3 | 0.0 |
| Bladder cancer NAT 4 | 0.6 |
| Prostate adenocarcinoma 1 | 0.9 |
| Prostate adenocarcinoma 2 | 0.1 |
| Prostate adenocarcinoma 3 | 0.1 |
| Prostate adenocarcinoma 4 | 0.5 |
| Prostate cancer NAT 5 | 0.2 |
| Prostate adenocarcinoma 6 | 0.1 |
| Prostate adenocarcinoma 7 | 0.0 |
| Prostate adenocarcinoma 8 | 0.1 |
| Prostate adenocarcinoma 9 | 1.5 |
| Prostate cancer NAT 10 | 0.1 |
| Kidney cancer 1 | 77.9 |
| Kidney NAT 1 | 5.7 |
| Kidney cancer 2 | 100.0 |
| Kidney NAT 2 | 3.4 |
| Kidney cancer 3 | 46.0 |
| Kidney NAT 3 | 2.4 |
| Kidney cancer 4 | 85.3 |
| Kidney NAT 4 | 1.2 |

Column A - Rel. Exp. (%) Ag1216 Run 259733296

AI_comprehensive panel_v1.0 Summary: Ag1216 Highest expression of the CG106951-01 and CG106951-04 genes was detected in a sample orthoarthritis bone (CT=31.2). Moderate to low levels of expression of these genes were detected in samples derived from normal and orthoarthitis/rheumatoid arthritis bone, cartilage, synovium and synovial fluid samples, as well as in normal lung, COPD lung, emphysema, atopic asthma, asthma, allergy, Crohn's disease (normal matched control and diseased), ulcerative colitis (normal matched control and diseased), and psoriasis (normal matched control and diseased). Therapeutic modulation of the activity of these genes or their protein products will ameliorate symptoms/conditions associated with autoimmune and inflammatory disorders including psoriasis, allergy, asthma, inflammatory bowel disease, rheumatoid arthritis and osteoarthritis.
General_screening_panel_v1.4 Summary: Ag1216 Highest expression of these genes was detected in renal cancer cell line 786-0 (CT=26.4). High to moderate expression of these genes was also seen in number of cancer cell lines derived from ovarian, breast, brain and kidney cancers. Therapeutic modulation of the activity of these genes or their protein products is useful in the treatment of these cancers.

Among tissues with metabolic or endocrine function, these genes were expressed at moderate to low levels in pancreas, adipose, adrenal gland, pituitary gland, skeletal muscle, heart, fetal liver and the gastrointestinal tract. Therapeutic modulation of the activity of these genes or their protein products is useful in the treatment of endocrine/metabolically related diseases, such as obesity and diabetes.

In addition, these genes were expressed at moderate levels in all regions of the central nervous system examined, including amygdala, hippocampus, substantia nigra, thalamus, cerebellum, cerebral cortex, and spinal cord. Therapeutic modulation of the activity of these genes or their protein products is useful in the treatment of central nervous system disorders such as Alzheimer's disease, Parkinson's disease, epilepsy, multiple sclerosis, schizophrenia and depression.

The CG106951-01 and CG106951-04 genes were also expressed at higher levels in fetal (CTs=29-32) liver, lung, heart, kidney and skeletal muscle when compared to adult tissues (CTs=33-37). The relative overexpression of these genes in fetal tissue suggests that the expressed proteins may enhance growth or development of these tissues in the fetus and thus may also act in a regenerative capacity in the adult. Therapeutic modulation of the activity of these genes or their protein products is useful in treatment of liver, lung, kidney, heart and skeletal muscle related diseases.

Panel 3D Summary: Ag1216 Moderate expression of these genes were detected mainly in a lung cancer DMS-79 cell line (CT=30.4). Therapeutic modulation of the activity of these genes or their protein products is useful in the treatment of lung cancer.

Panel 4D Summary: Ag1216 Highest expression of these genes was detected in thymus (CTs=31-32). These genes also show low expression in normal lung as well as in astrocytes and bronchial epithelium treated with TNF-α and IL-1β. Therapeutic modulation of the activity of these genes or their protein products is useful in the treatment of inflammatory diseases including asthma, allergies, inflammatory bowel disease, lupus erythematosus, psoriasis, rheumatoid arthritis, and osteoarthritis.

Panel 5 Islet Summary: Ag1216 Highest expression of these genes were detected in placenta (CT=34). Low expression of these genes was also seen in adipose and uterus. Please see panel 1.4 for further discussion of these genes.

General oncology screening panel_v_{—}2.4 Summary: Ag1216 Highest expression of these genes was detected in a kidney cancer sample (CT=27). Expression of these genes was higher in 4/4 kidney cancer, 3/3 colon cancer, and 3/3 lung cancer samples relative to corresponding normal adjacent tissue. In addition, significant expression of these genes was also seen in metastatic melanoma and prostate cancers. Gene or protein expression levels are useful as a marker to detect the presence of these cancers. Therapeutic modulation of the activity of these genes or their protein products using nucleic acid, protein, antibody or small molecule drugs is useful in the treatment of kidney, lung, colon, metastatic melanoma and prostate cancers.

C. CG124756-01: Complement Component 1, q Subcomponent, Beta Polypeptide.

Expression of gene CG124756-01 was assessed using the primer-probe set Ag4901, described in Table CA. Results of the RTQ-PCR runs are shown in Tables CB and CC.

TABLE CA
Probe Name Ag4901

| | | | Start | SEQ ID |
|---|---|---|---|---|
| Primers | Sequences | Length | Position | No |
| Forward | 5′-ccacgtgatcaccaac | 19 | 503 | 289 |
| | atg-3′ | | | |
| Probe | TET-5′-aacaacaattatga | 25 | 522 | 290 |
| | gccccgcagtg-3′-TAMRA | | | |
| Reverse | 5′-tggcgtggtaggtgaagt | 22 | 576 | 291 |
| | agta-3′ | | | |

TABLE CB
CNS neurodegeneration v1.0
Column A - Rel. Exp. (%) Ag4901, 224996029

| Tissue Name | A |
|---|---|
| AD 1 Hippo | 77.9 |
| AD 2 Hippo | 72.7 |
| AD 3 Hippo | 39.0 |
| AD 4 Hippo | 23.3 |
| AD 5 Hippo | 30.6 |
| AD 6 Hippo | 7.6 |
| Control 2 Hippo | 63.3 |
| Control 4 Hippo | 65.5 |
| Control (Path) 3 Hippo | 23.8 |
| AD 1 Temporal Ctx | 62.9 |
| AD 2 Temporal Ctx | 43.8 |
| AD 3 Temporal Ctx | 25.9 |
| AD 4 Temporal Ctx | 33.0 |
| AD 5 Inf Temporal Ctx | 30.8 |
| AD 5 Sup Temporal Ctx | 71.7 |
| AD 6 Inf Temporal Ctx | 100.0 |
| AD 6 Sup Temporal Ctx | 79.6 |
| Control 1 Temporal Ctx | 22.8 |
| Control 2 Temporal Ctx | 53.2 |
| Control 3 Temporal Ctx | 49.3 |
| Control 3 Temporal Ctx | 16.0 |
| Control (Path) 1 Temporal Ctx | 18.3 |
| Control (Path) 2 Temporal Ctx | 21.8 |
| Control (Path) 3 Temporal Ctx | 10.2 |
| Control (Path) 4 Temporal Ctx | 17.4 |
| AD 1 Occipital Ctx | 39.8 |
| AD 2 Occipital Ctx (Missing) | 0.4 |
| AD 3 Occipital Ctx | 13.2 |
| AD 4 Occipital Ctx | 18.6 |
| AD 5 Occipital Ctx | 39.8 |
| AD 6 Occipital Ctx | 20.2 |
| Control 1 Occipital Ctx | 7.1 |
| Control 2 Occipital Ctx | 21.9 |
| Control 3 Occipital Ctx | 24.7 |
| Control 4 Occipital Ctx | 27.5 |
| Control (Path) 1 Occipital Ctx | 11.0 |
| Control (Path) 2 Occipital Ctx | 11.7 |
| Control (Path) 3 Occipital Ctx | 0.9 |
| Control (Path) 4 Occipital Ctx | 15.8 |
| Control 1 Parietal Ctx | 26.8 |
| Control 2 Parietal Ctx | 38.4 |
| Control 3 Parietal Ctx | 27.7 |
| Control (Path) 1 Parietal Ctx | 12.5 |
| Control (Path) 2 Parietal Ctx | 14.0 |
| Control (Path) 3 Parietal Ctx | 3.0 |
| Control (Path) 4 Parietal Ctx | 19.9 |

TABLE CC
General screening panel v1.6
Column A - Rel. Exp. (%) Ag4901, Run 277231336

| Tissue Name | A |
|---|---|
| Adipose | 35.8 |
| Melanoma* Hs688(A).T | 0.0 |
| Melanoma* Hs688(B).T | 0.0 |
| Melanoma* M14 | 0.0 |
| Melanoma* LOXIMVI | 0.0 |
| Melanoma* SK-MEL-5 | 0.0 |
| Squamous cell carcinoma SCC-4 | 0.0 |
| Testis Pool | 9.9 |
| Prostate ca.* (bone met) PC-3 | 0.0 |
| Prostate Pool | 11.5 |
| Placenta | 73.2 |
| Uterus Pool | 1.0 |
| Ovarian ca. OVCAR-3 | 0.0 |
| Ovarian ca. SK-OV-3 | 0.0 |
| Ovarian ca. OVCAR-4 | 0.0 |
| Ovarian ca. OVCAR-5 | 0.0 |
| Ovarian ca. IGROV-1 | 0.0 |
| Ovarian ca. OVCAR-8 | 0.0 |
| Ovary | 21.6 |
| Breast ca. MCF-7 | 0.0 |
| Breast ca. MDA-MB-231 | 0.0 |
| Breast ca. BT 549 | 0.0 |
| Breast ca. T47D | 0.0 |
| Breast ca. MDA-N | 0.0 |
| Breast Pool | 8.0 |
| Trachea | 19.8 |
| Lung | 2.3 |
| Fetal Lung | 13.9 |
| Lung ca. NCI-N417 | 0.0 |
| Lung ca. LX-1 | 0.0 |
| Lung ca. NCI-H146 | 0.0 |
| Lung ca. SHP-77 | 0.0 |
| Lung ca. A549 | 0.0 |
| Lung ca. NCI-H526 | 0.0 |
| Lung ca. NCI-H23 | 0.0 |
| Lung ca. NCI-H460 | 0.0 |
| Lung ca. HOP-62 | 0.0 |
| Lung ca. NCI-H522 | 0.0 |
| Liver | 18.4 |
| Fetal Liver | 55.9 |
| Liver ca. HepG2 | 0.0 |
| Kidney Pool | 14.1 |
| Fetal Kidney | 4.7 |
| Renal ca. 786-0 | 0.0 |
| Renal ca. A498 | 0.0 |
| Renal ca. ACHN | 0.0 |
| Renal ca. UO-31 | 0.0 |
| Renal ca. TK-10 | 0.0 |
| Bladder | 100.0 |
| Gastric ca. (liver met.) NCI-N87 | 0.0 |
| Gastric ca. KATO III | 0.0 |
| Colon ca. SW-948 | 0.0 |
| Colon ca. SW480 | 0.0 |
| Colon ca.* (SW480 met) SW620 | 0.0 |
| Colon ca. HT29 | 0.0 |
| Colon ca. HCT-116 | 0.0 |
| Colon ca. CaCo-2 | 0.0 |
| Colon cancer tissue | 75.8 |
| Colon ca. SW1116 | 0.0 |
| Colon ca. Colo-205 | 0.2 |
| Colon ca. SW-48 | 0.0 |
| Colon Pool | 11.6 |
| Small Intestine Pool | 6.8 |
| Stomach Pool | 14.8 |
| Bone Marrow Pool | 12.7 |
| Fetal Heart | 1.9 |
| Heart Pool | 5.1 |
| Lymph Node Pool | 11.1 |
| Fetal Skeletal Muscle | 7.3 |
| Skeletal Muscle Pool | 2.0 |
| Spleen Pool | 27.2 |
| Thymus Pool | 13.2 |
| CNS cancer (glio/astro) U87-MG | 0.0 |
| CNS cancer (glio/astro) U-118-MG | 0.1 |
| CNS cancer (neuro; met) SK-N-AS | 0.0 |
| CNS cancer (astro) SF-539 | 0.0 |
| CNS cancer (astro) SNB-75 | 0.0 |
| CNS cancer (glio) SNB-19 | 0.0 |
| CNS cancer (glio) SF-295 | 0.0 |
| Brain (Amygdala) Pool | 7.8 |
| Brain (cerebellum) | 26.8 |
| Brain (fetal) | 8.1 |
| Brain (Hippocampus) Pool | 13.2 |
| Cerebral Cortex Pool | 8.2 |
| Brain (Substantia nigra) Pool | 8.4 |
| Brain (Thalamus) Pool | 8.3 |
| Brain (whole) | 22.4 |
| Spinal Cord Pool | 18.3 |
| Adrenal Gland | 55.1 |
| Pituitary gland Pool | 3.9 |
| Salivary Gland | 5.8 |
| Thyroid (female) | 6.8 |
| Pancreatic ca. CAPAN2 | 0.0 |
| Pancreas Pool | 6.9 |

CNS_neurodegeneration_v10 Summary: Ag4901 Expression of the CG124756-01 gene was upregulated in the temporal cortex of Alzheimer's disease patients compared to normal patients. Inhibition of this gene or its protein product is useful in the treatment of Alzheimer's disease and can decrease neuronal death.
General_screening_panel_v1.6 Summary: Ag4901 The highest expression of this gene was detected in bladder (CT=26). In addition, this gene was expressed at high to moderate levels in pancreas, adipose, adrenal gland, thyroid, pituitary gland, skeletal muscle, heart, liver and the gastrointestinal tract. Therapeutic modulation of the activity of this gene or its protein product is useful in the treatment of endocrine/metabolically related diseases, such as obesity and diabetes.

This gene was also expressed at moderate levels in all regions of the central nervous system examined, including amygdala, hippocampus, substantia nigra, thalamus, cerebellum, cerebral cortex, and spinal cord. Thereapeutic modulation of the activity of this gene or its protein product using nucleic acid, protein, antibody or small molecule drugs is useful in the treatment of central nervous system disorders such as Alzheimer's disease, Parkinson's disease, epilepsy, multiple sclerosis, schizophrenia and depression.

High expression of this gene was also seen in a colon cancer cell line. Therapeutic modulation of the activity of this gene or its protein product is useful in the treatment of colon cancer.

D. CG50353-01: 129293352_EXT, Wnt 7a like Protein.

Expression of gene CG50353-01 was assessed using the primer-probe set Ag3093, described in Table DA. Results of the RTQ-PCR runs are shown in Tables DB and DC.

TABLE DA
Probe Name Ag3093

| | | | Start | SEQ ID |
|---|---|---|---|---|
| Primers | Sequences | Length | Position | No |
| Forward | 5′-ctgtgacctcatgtgct | 20 | 909 | 292 |
| | gtg-3′ | | | |
| Probe | TET-5′-gtggctacaacacc | 25 | 932 | 293 |
| | caccagtacgc-3′-TAMRA | | | |
| Reverse | 5′-acatagcagcaccagtg | 20 | 982 | 294 |
| | gaa-3′ | | | |

TABLE DB
Panel 1.3D
Column A - Rel. Exp. (%) Ag3093, Run 167985246

| Tissue Name | A |
|---|---|
| Liver adenocarcinoma | 2.8 |
| Pancreas | 0.0 |
| Pancreatic ca. CAPAN 2 | 1.7 |
| Adrenal gland | 0.0 |
| Thyroid | 0.0 |
| Salivary gland | 0.0 |
| Pituitary gland | 0.0 |
| Brain (fetal) | 3.6 |
| Brain (whole) | 1.5 |
| Brain (amygdala) | 1.8 |
| Brain (cerebellum) | 0.9 |
| Brain (hippocampus) | 1.4 |
| Brain (substantia nigra) | 0.9 |
| Brain (thalamus) | 0.0 |
| Cerebral Cortex | 3.5 |
| Spinal cord | 0.6 |
| glio/astro U87-MG | 0.6 |
| glio/astro U-118-MG | 0.0 |
| astrocytoma SW1783 | 0.0 |
| neuro*; met SK-N-AS | 0.0 |
| astrocytoma SF-539 | 0.2 |
| astrocytoma SNB-75 | 0.1 |
| glioma SNB-19 | 0.0 |
| glioma U251 | 0.0 |
| glioma SF-295 | 0.0 |
| Heart (fetal) | 0.0 |
| Heart | 0.0 |
| Skeletal muscle (fetal) | 0.0 |
| Skeletal muscle | 0.0 |
| Bone marrow | 0.0 |
| Thymus | 0.0 |
| Spleen | 0.3 |
| Lymph node | 0.0 |
| Colorectal | 0.0 |
| Stomach | 0.0 |
| Small intestine | 0.0 |
| Colon ca. SW480 | 0.4 |
| Colon ca.* SW620(SW480 met) | 1.4 |
| Colon ca. HT29 | 0.0 |
| Colon ca. HCT-116 | 0.0 |
| Colon ca. CaCo-2 | 0.2 |
| Colon ca. tissue(ODO3866) | 0.0 |
| Colon ca. HCC-2998 | 0.1 |
| Gastric ca.* (liver met) NCI-N87 | 0.5 |
| Bladder | 0.0 |
| Trachea | 0.1 |
| Kidney | 0.0 |
| Kidney (fetal) | 0.1 |
| Renal ca. 786-0 | 0.2 |
| Renal ca. A498 | 0.0 |
| Renal ca. RXF 393 | 0.4 |
| Renal ca. ACHN | 0.0 |
| Renal ca. UO-31 | 0.5 |
| Renal ca. TK-10 | 0.0 |
| Liver | 0.0 |
| Liver (fetal) | 0.0 |
| Liver ca. (hepatoblast) HepG2 | 0.0 |
| Lung | 0.2 |
| Lung (fetal) | 0.9 |
| Lung ca. (small cell) LX-1 | 0.0 |
| Lung ca. (small cell) NCI-H69 | 0.2 |
| Lung ca. (s. cell var.) SHP-77 | 0.0 |
| Lung ca. (large cell)NCI-H460 | 0.0 |
| Lung ca. (non-sm. cell) A549 | 0.2 |
| Lung ca. (non-s. cell) NCI-H23 | 0.0 |
| Lung ca. (non-s. cell) HOP-62 | 0.0 |
| Lung ca. (non-s. cl) NCI-H522 | 0.0 |
| Lung ca. (squam.) SW 900 | 0.0 |
| Lung ca. (squam.) NCI-H596 | 0.3 |
| Mammary gland | 0.0 |
| Breast ca.* (pl. ef) MCF-7 | 0.2 |
| Breast ca.* (pl. ef) MDA-MB-231 | 0.0 |
| Breast ca.* (pl. ef) T47D | 0.0 |
| Breast ca. BT-549 | 0.0 |
| Breast ca. MDA-N | 0.0 |
| Ovary | 0.0 |
| Ovarian ca. OVCAR-3 | 0.1 |
| Ovarian ca. OVCAR-4 | 37.1 |
| Ovarian ca. OVCAR-5 | 0.7 |
| Ovarian ca. OVCAR-8 | 0.0 |
| Ovarian ca. IGROV-1 | 6.8 |
| Ovarian ca.* (ascites) SK-OV-3 | 100.0 |
| Uterus | 0.0 |
| Placenta | 0.0 |
| Prostate | 0.0 |
| Prostate ca.* (bone met)PC-3 | 2.0 |
| Testis | 0.3 |
| Melanoma Hs688(A).T | 0.0 |
| Melanoma* (met) Hs688(B).T | 0.0 |
| Melanoma UACC-62 | 0.0 |
| Melanoma M14 | 0.0 |
| Melanoma LOX IMVI | 0.0 |
| Melanoma* (met) SK-MEL-5 | 0.0 |
| Adipose | 0.2 |

TABLE DC
Panel 4D
Column A - Rel. Exp. (%) Ag3093, Run 164392077

| Tissue Name | A |
|---|---|
| Secondary Th1 act | 0.0 |
| Secondary Th2 act | 0.0 |
| Secondary Tr1 act | 0.0 |
| Secondary Th1 rest | 0.0 |
| Secondary Th2 rest | 0.0 |
| Secondary Tr1 rest | 0.0 |
| Primary Th1 act | 0.0 |
| Primary Th2 act | 0.0 |
| Primary Tr1 act | 0.0 |
| Primary Th1 rest | 0.0 |
| Primary Th2 rest | 0.0 |
| Primary Tr1 rest | 0.0 |
| CD45RA CD4 lymphocyte act | 0.0 |
| CD45RO CD4 lymphocyte act | 0.0 |
| CD8 lymphocyte act | 0.0 |
| Secondary CD8 lymphocyte rest | 0.0 |
| Secondary CD8 lymphocyte act | 0.0 |
| CD4 lymphocyte none | 4.9 |
| 2ry Th1/Th2/Tr1_anti-CD95 CH11 | 0.0 |
| LAK cells rest | 0.0 |
| LAK cells IL-2 | 0.0 |
| LAK cells IL-2 + IL-12 | 0.0 |
| LAK cells IL-2 + IFN gamma | 0.0 |
| LAK cells IL-2 + IL-18 | 0.0 |
| LAK cells PMA/ionomycin | 0.0 |
| NK Cells IL-2 rest | 0.0 |
| Two Way MLR 3 day | 0.0 |
| Two Way MLR 5 day | 0.0 |
| Two Way MLR 7 day | 0.0 |
| PBMC rest | 3.5 |
| PBMC PWM | 0.8 |
| PBMC PHA-L | 0.0 |
| Ramos (B cell) none | 0.0 |
| Ramos (B cell) ionomycin | 0.0 |
| B lymphocytes PWM | 0.0 |
| B lymphocytes CD40L and IL-4 | 0.0 |
| EOL-1 dbcAMP | 0.0 |
| EOL-1 dbcAMP PMA/ionomycin | 0.0 |
| Dendritic cells none | 0.0 |
| Dendritic cells LPS | 0.0 |
| Dendritic cells anti-CD40 | 0.0 |
| Monocytes rest | 0.0 |
| Monocytes LPS | 0.0 |
| Macrophages rest | 0.0 |
| Macrophages LPS | 0.0 |
| HUVEC none | 0.0 |
| HUVEC starved | 0.0 |
| HUVEC IL-1beta | 0.0 |
| HUVEC IFN gamma | 0.0 |
| HUVEC TNF alpha + IFN gamma | 0.0 |
| HUVEC TNF alpha + IL4 | 0.0 |
| HUVEC IL-11 | 0.0 |
| Lung Microvascular EC none | 0.0 |
| Lung Microvascular EC TNFalpha + IL-1beta | 0.0 |
| Microvascular Dermal EC none | 0.0 |
| Microsvasular Dermal EC TNFalpha + IL-1beta | 0.0 |
| Bronchial epithelium TNFalpha + IL1beta | 57.4 |
| Small airway epithelium none | 17.7 |
| Small airway epithelium TNFalpha + IL-1beta | 100.0 |
| Coronery artery SMC rest | 0.0 |
| Coronery artery SMC TNFalpha + IL-1beta | 0.0 |
| Astrocytes rest | 0.0 |
| Astrocytes TNFalpha + IL-1beta | 0.0 |
| KU-812 (Basophil) rest | 0.0 |
| KU-812 (Basophil) PMA/ionomycin | 1.2 |
| CCD1106 (Keratinocytes) none | 47.6 |
| CCD1106 (Keratinocytes) TNFalpha + IL-1beta | 33.7 |
| Liver cirrhosis | 1.4 |
| Lupus kidney | 0.0 |
| NCI-H292 none | 4.1 |
| NCI-H292 IL-4 | 4.8 |
| NCI-H292 IL-9 | 1.8 |
| NCI-H292 IL-13 | 2.5 |
| NCI-H292 IFN gamma | 1.6 |
| HPAEC none | 0.0 |
| HPAEC TNF alpha + IL-1 beta | 0.0 |
| Lung fibroblast none | 0.0 |
| Lung fibroblast TNF alpha + IL-1 beta | 0.0 |
| Lung fibroblast IL-4 | 0.0 |
| Lung fibroblast IL-9 | 0.0 |
| Lung fibroblast IL-13 | 0.0 |
| Lung fibroblast IFN gamma | 0.0 |
| Dermal fibroblast CCD1070 rest | 0.0 |
| Dermal fibroblast CCD1070 TNF alpha | 0.0 |
| Dermal fibroblast CCD1070 IL-1 beta | 0.0 |
| Dermal fibroblast IFN gamma | 0.0 |
| Dermal fibroblast IL-4 | 0.0 |
| IBD Colitis 2 | 0.0 |
| IBD Crohn's | 0.0 |
| Colon | 1.0 |
| Lung | 2.0 |
| Thymus | 0.0 |
| Kidney | 0.0 |

Panel 1.3D Summary: Ag3093 Highest expression of the CG50353-01 gene was detected in the SK-OV-3 ovarian cancer cell line derived from ascites fluid (CT=30.28). This gene was also expressed in two additional ovarian cancer cell lines. Gene or protein expression levels are useful as a marker for ovarian cancer or for ascites. Therapeutic modulation of the activity of this gene or its protein product using nucleic acid, protein, antibody or small molecule drugs is useful in the treatment of ovarian cancer.
Panel 4D Summary: This gene was expressed at the highest level in TNF alpha+IL-1 beta-treated small airway epithelial cells (CT=32.6) and bronchial epithelial cells as well as in CCD1106 keratinocytes, independent of treatment. Expression of this gene in keratinocytes suggests that it is important in skin disorders including psoriasis. Expression of this gene in airway/bronchial cell types suggests that this gene also plays a role in inflammatory lung disorders, including, for example, chronic obstructive pulmonary disease (COPD), asthma, allergy and emphysema. Therapeutic modulation of the activity of this gene or its protein product is useful in the treatment of skin disorders, such as psoriasis, and inflammatory lung disorders, including COPD, asthma, allergy and emphysema.

E. CG50709-03 and CG50709-05: WNT14B

Expression of genes CG50709-03 and CG50709-05 was assessed using the primer-probe sets Ag2262, and Ag2316, described in Tables EA, and EB. Results of the RTQ-PCR runs are shown in Tables EC, ED, EE, and EF.

TABLE EA
Probe Name Ag2262

| | | | Start | SEQ ID |
|---|---|---|---|---|
| Primers | Sequences | Length | Position | No |
| Forward | 5′-gacctggtgtacatgga | 20 | 875 | 295 |
| | gga-3′ | | | |
| Probe | TET-5′-cttctgccggccca | 23 | 904 | 296 |
| | gcaagtact-3′-TAMRA | | | |
| Reverse | 5′-gagcacaccctacctg | 19 | 936 | 297 |
| | ctg-3′ | | | |

TABLE EB
Probe Name Ag2316

| | | | Start | SEQ ID |
|---|---|---|---|---|
| Primers | Sequences | Length | Position | No |
| Forward | 5′-gtccaagagaggaaacaa | 21 | 571 | 298 |
| | gga-3′ | | | |
| Probe | TET-5′-cacaatacccacgt | 24 | 614 | 299 |
| | gggcatcaag-3′-TAMRA | | | |
| Reverse | 5′-gtcctgaggccactctt | 20 | 641 | 300 |
| | cac-3′ | | | |

TABLE EC
Ardais Kidney 1.0
Column A - el. Exp. (%) Ag2262, Run 369787102

| Tissue Name | A |
|---|---|
| Kidney cancer(10A8) | 0.0 |
| Kidney NAT(10A9) | 1.1 |
| Kidney cancer(10AA) | 8.9 |
| Kidney NAT(10AB) | 15.3 |
| Kidney cancer(10AC) | 2.2 |
| Kidney NAT(10AD) | 100.0 |
| Kidney cancer(10B6) | 1.8 |
| Kidney NAT(10B7) | 4.5 |
| Kidney cancer(10B8) | 0.3 |
| Kidney NAT(10B9) | 4.1 |
| Kidney cancer(10BC) | 5.1 |
| Kidney NAT(10BD) | 54.7 |
| Kidney cancer(10BE) | 0.2 |
| Kidney NAT(10BF) | 32.3 |
| Kidney cancer(10C2) | 0.1 |
| Kidney NAT(10C3) | 11.7 |
| Kidney cancer(10C4) | 0.0 |
| Kidney NAT(10C5) | 8.2 |
| Kidney cancer(10B4) | 0.5 |
| Kidney cancer(10C8) | 1.3 |
| Kidney cancer(10D0) | 0.9 |
| Kidney cancer(10C0) | 0.3 |
| Kidney cancer(10C6) | 0.0 |
| Kidney cancer(10C9) | 0.0 |
| Kidney cancer(10D1) | 4.0 |
| Kidney cancer(10CA) | 1.9 |
| Kidney cancer(10D2) | 2.2 |
| Kidney cancer(10CB) | 1.0 |
| Kidney cancer(10D4) | 1.8 |
| Kidney cancer(10CD) | 1.5 |
| Kidney cancer(10D5) | 0.0 |
| Kidney cancer(10CE) | 2.6 |
| Kidney cancer(10D6) | 1.0 |
| Kidney cancer(10CF) | 1.2 |
| Kidney cancer(10D8) | 1.1 |
| Kidney cancer(10CC) | 1.2 |
| Kidney cancer(10D3) | 4.2 |
| Kidney NAT(10D9) | 15.2 |
| Kidney NAT(10DB) | 19.5 |
| Kidney NAT(10DC) | 8.7 |
| Kidney NAT(10DD) | 22.1 |
| Kidney NAT(10DE) | 10.2 |
| Kidney NAT(10B1) | 2.7 |
| Kidney NAT(10DA) | 19.3 |

TABLE ED
Panel 1.3D

| Tissue Name | A | B | C |
|---|---|---|---|
| Liver adenocarcinoma | 0.0 | 6.7 | 0.0 |
| Pancreas | 0.0 | 0.0 | 0.0 |
| Pancreatic ca. CAPAN 2 | 0.0 | 0.0 | 0.0 |
| Adrenal gland | 1.9 | 0.0 | 0.0 |
| Thyroid | 2.2 | 0.0 | 0.0 |
| Salivary gland | 0.3 | 0.0 | 0.0 |
| Pituitary gland | 0.0 | 8.0 | 0.0 |
| Brain (fetal) | 0.0 | 1.0 | 0.0 |
| Brain (whole) | 5.2 | 0.0 | 26.2 |
| Brain (amygdala) | 6.8 | 3.8 | 11.5 |
| Brain (cerebellum) | 1.0 | 6.4 | 0.0 |
| Brain (hippocampus) | 16.5 | 0.0 | 0.0 |
| Brain (substantia nigra) | 2.0 | 0.0 | 0.0 |
| Brain (thalamus) | 4.9 | 11.2 | 57.0 |
| Cerebral Cortex | 2.5 | 13.3 | 3.3 |
| Spinal cord | 3.3 | 9.2 | 6.8 |
| glio/astro U87-MG | 0.0 | 0.0 | 0.0 |
| glio/astro U-118-MG | 0.0 | 0.0 | 0.0 |
| Astrocytoma SW1783 | 0.0 | 0.0 | 0.0 |
| neuro*; met SK-N-AS | 0.0 | 0.0 | 0.0 |
| astrocytoma SF-539 | 0.0 | 0.0 | 0.0 |
| astrocytoma SNB-75 | 0.0 | 0.0 | 0.0 |
| glioma SNB-19 | 0.0 | 0.0 | 0.0 |
| glioma U251 | 0.0 | 0.0 | 0.0 |
| glioma SF-295 | 0.0 | 0.0 | 0.0 |
| Heart (fetal) | 2.0 | 0.0 | 33.4 |
| Heart | 0.0 | 6.7 | 9.6 |
| Skeletal muscle (fetal) | 2.5 | 0.0 | 8.2 |
| Skeletal muscle | 0.0 | 0.0 | 0.0 |
| Bone marrow | 0.9 | 0.0 | 0.0 |
| Thymus | 0.0 | 0.0 | 0.0 |
| Spleen | 100.0 | 65.5 | 100.0 |
| Lymph node | 0.0 | 0.0 | 0.0 |
| Colorectal | 10.8 | 19.8 | 0.0 |
| Stomach | 2.7 | 0.0 | 0.0 |
| Small intestine | 6.4 | 0.0 | 0.0 |
| Colon ca. SW480 | 0.0 | 0.0 | 0.0 |
| Colon ca.* SW620(SW480 met) | 1.2 | 0.0 | 0.0 |
| Colon ca. HT29 | 0.0 | 0.0 | 0.0 |
| Colon ca. HCT-116 | 0.0 | 0.0 | 0.0 |
| Colon ca. CaCo-2 | 2.5 | 6.6 | 0.0 |
| Colon ca. tissue(ODO3866) | 0.0 | 0.0 | 0.0 |
| Colon ca. HCC-2998 | 0.0 | 0.0 | 0.0 |
| Gastric ca.* (liver met) NCI-N87 | 0.0 | 14.7 | 0.0 |
| Bladder | 0.0 | 6.5 | 16.2 |
| Trachea | 5.0 | 0.0 | 6.0 |
| Kidney | 14.9 | 7.9 | 31.0 |
| Kidney (fetal) | 24.0 | 100.0 | 50.0 |
| Renal ca. 786-0 | 0.0 | 0.0 | 0.0 |
| Renal ca. A498 | 0.0 | 12.6 | 0.0 |
| Renal ca. RXF 393 | 0.0 | 0.0 | 0.0 |
| Renal ca. ACHN | 0.0 | 0.0 | 0.0 |
| Renal ca. UO-31 | 0.2 | 0.0 | 0.0 |
| Renal ca. TK-10 | 0.0 | 0.0 | 0.0 |
| Liver | 0.0 | 0.0 | 0.0 |
| Liver (fetal) | 0.0 | 0.0 | 0.0 |
| Liver ca. (hepatoblast) HepG2 | 0.0 | 0.0 | 0.0 |
| Lung | 6.8 | 0.0 | 19.3 |
| Lung (fetal) | 8.5 | 0.0 | 6.8 |
| Lung ca. (small cell) LX-1 | 0.0 | 0.0 | 0.0 |
| Lung ca. (small cell) NCI-H69 | 0.3 | 0.0 | 0.0 |
| Lung ca. (s. cell var.) SHP-77 | 2.5 | 6.9 | 0.0 |
| Lung ca. (large cell) NCI-H460 | 0.0 | 0.0 | 0.0 |
| Lung ca. (non-sm. cell) A549 | 0.0 | 6.4 | 0.0 |
| Lung ca. (non-s. cell) NCI-H23 | 0.0 | 0.0 | 0.0 |
| Lung ca. (non-s. cell) HOP-62 | 0.0 | 0.0 | 0.0 |
| Lungca. (non-s. cl) NCI-H522 | 2.8 | 0.0 | 0.0 |
| Lung ca. (squam.) SW 900 | 0.0 | 0.0 | 0.0 |
| Lung ca. (squam.) NCI-H596 | 0.0 | 0.0 | 0.0 |
| Mammary gland | 0.0 | 0.0 | 0.0 |
| Breast ca.* (pl. ef) MCF-7 | 0.0 | 0.0 | 0.0 |
| Breast ca.* (pl. ef) | 0.0 | 0.0 | 0.0 |
| MDA-MB-231 | | | |
| Breast ca.* (pl. ef) T47D | 0.0 | 0.0 | 0.0 |
| Breast ca. BT-549 | 0.0 | 0.0 | 0.0 |
| Breast ca. MDA-N | 1.0 | 0.0 | 0.0 |
| Ovary | 0.0 | 0.0 | 6.4 |
| Ovarian ca. OVCAR-3 | 0.0 | 0.0 | 0.0 |
| Ovarian ca. OVCAR-4 | 0.0 | 0.0 | 0.0 |
| Ovarian ca. OVCAR-5 | 0.0 | 0.0 | 0.0 |
| Ovarian ca. OVCAR-8 | 0.0 | 0.0 | 0.0 |
| Ovarian ca. IGROV-1 | 0.0 | 0.0 | 0.0 |
| Ovarian ca.* (ascites) | 0.0 | 0.0 | 0.0 |
| SK-OV-3 | | | |
| Uterus | 0.0 | 0.0 | 0.0 |
| Placenta | 0.6 | 7.1 | 0.0 |
| Prostate | 0.0 | 1.8 | 4.9 |
| Prostate ca.* (bone met) PC-3 | 0.0 | 0.0 | 0.0 |
| Testis | 1.7 | 0.0 | 7.2 |
| Melanoma Hs688(A).T | 0.0 | 0.0 | 0.0 |
| Melanoma* (met) Hs688(B).T | 0.0 | 0.0 | 0.0 |
| Melanoma UACC-62 | 0.0 | 0.0 | 0.0 |
| Melanoma M14 | 0.0 | 0.0 | 0.0 |
| Melanoma LOX IMVI | 0.0 | 0.0 | 0.0 |
| Melanoma* (met) SK-MEL-5 | 0.0 | 0.0 | 0.0 |
| Adipose | 0.0 | 0.0 | 7.6 |

Column A - Rel. Exp. (%) Ag2262, Run 150719071
Column B - Rel. Exp. (%) Ag2262, Run 167966858
Column C - Rel. Exp. (%) Ag2316, Run 162185396

TABLE EE
Panel 2D
Column A - Rel. Exp. (%) Ag2262, Run 150943107

| Tissue Name | A |
|---|---|
| Normal Colon | 14.2 |
| CC Well to Mod Diff (ODO3866) | 14.2 |
| CC Margin (ODO3866) | 0.0 |
| CC Gr. 2 rectosigmoid (ODO3868) | 0.0 |
| CC Margin (ODO3868) | 0.0 |
| CC Mod Diff (ODO3920) | 0.0 |
| CC Margin (ODO3920) | 0.8 |
| CC Gr. 2 ascend colon (ODO3921) | 0.0 |
| CC Margin (ODO3921) | 0.9 |
| CC from Partial Hepatectomy (ODO4309) | 0.0 |
| Mets | |
| Liver Margin (ODO4309) | 1.1 |
| Colon mets to lung (OD04451-01) | 7.3 |
| Lung Margin (OD04451-02) | 0.0 |
| Normal Prostate 6546-1 | 18.6 |
| Prostate Cancer (OD04410) | 10.2 |
| Prostate Margin (OD04410) | 0.0 |
| Prostate Cancer (OD04720-01) | 0.0 |
| Prostate Margin (OD04720-02) | 9.8 |
| Normal Lung 061010 | 22.5 |
| Lung Met to Muscle (ODO4286) | 6.1 |
| Muscle Margin (ODO4286) | 0.0 |
| Lung Malignant Cancer (OD03126) | 5.4 |
| Lung Margin (OD03126) | 0.0 |
| Lung Cancer (OD04404) | 7.6 |
| Lung Margin (OD04404) | 3.8 |
| Lung Cancer (OD04565) | 0.0 |
| Lung Margin (OD04565) | 0.0 |
| Lung Cancer (OD04237-01) | 0.0 |
| Lung Margin (OD04237-02) | 6.9 |
| Ocular Mel Met to Liver (ODO4310) | 1.1 |
| Liver Margin (ODO4310) | 28.5 |
| Melanoma Mets to Lung (OD04321) | 0.0 |
| Lung Margin (OD04321) | 0.0 |
| Normal Kidney | 100.0 |
| Kidney Ca, Nuclear grade 2 (OD04338) | 15.2 |
| Kidney Margin (OD04338) | 40.3 |
| Kidney Ca Nuclear grade ½ (OD04339) | 0.0 |
| Kidney Margin (OD04339) | 50.0 |
| Kidney Ca, Clear cell type (OD04340) | 0.0 |
| Kidney Margin (OD04340) | 31.2 |
| Kidney Ca, Nuclear grade 3 (OD04348) | 0.0 |
| Kidney Margin (OD04348) | 29.9 |
| Kidney Cancer (OD04622-01) | 0.0 |
| Kidney Margin (OD04622-03) | 58.6 |
| Kidney Cancer (OD04450-01) | 0.0 |
| Kidney Margin (OD04450-03) | 95.9 |
| Kidney Cancer 8120607 | 0.0 |
| Kidney Margin 8120608 | 24.0 |
| Kidney Cancer 8120613 | 0.0 |
| Kidney Margin 8120614 | 46.3 |
| Kidney Cancer 9010320 | 0.0 |
| Kidney Margin 9010321 | 16.5 |
| Normal Uterus | 16.4 |
| Uterus Cancer 064011 | 0.0 |
| Normal Thyroid | 15.6 |
| Thyroid Cancer 064010 | 0.0 |
| Thyroid Cancer A302152 | 6.8 |
| Thyroid Margin A302153 | 0.0 |
| Normal Breast | 9.3 |
| Breast Cancer (OD04566) | 0.0 |
| Breast Cancer (OD04590-01) | 4.8 |
| Breast Cancer Mets (OD04590-03) | 8.5 |
| Breast Cancer Metastasis | 0.0 |
| (OD04655-05) | |
| Breast Cancer 064006 | 7.2 |
| Breast Cancer 1024 | 0.0 |
| Breast Cancer 9100266 | 0.7 |
| Breast Margin 9100265 | 0.0 |
| Breast Cancer A209073 | 0.0 |
| Breast Margin A209073 | 0.0 |
| Normal Liver | 0.0 |
| Liver Cancer 064003 | 0.0 |
| Liver Cancer 1025 | 5.6 |
| Liver Cancer 1026 | 2.4 |
| Liver Cancer 6004-T | 0.0 |
| Liver Tissue 6004-N | 8.7 |
| Liver Cancer 6005-T | 0.0 |
| Liver Tissue 6005-N | 0.0 |
| Normal Bladder | 0.0 |
| Bladder Cancer 1023 | 0.0 |
| Bladder Cancer A302173 | 18.3 |
| Bladder Cancer (OD04718-01) | 0.0 |
| Bladder Normal Adjacent | 0.0 |
| (OD04718-03) | |
| Normal Ovary | 0.0 |
| Ovarian Cancer 064008 | 7.5 |
| Ovarian Cancer (OD04768-07) | 0.0 |
| Ovary Margin (OD04768-08) | 0.0 |
| Normal Stomach | 13.8 |
| Gastric Cancer 9060358 | 0.0 |
| Stomach Margin 9060359 | 0.0 |
| Gastric Cancer 9060395 | 0.0 |
| Stomach Margin 9060394 | 0.0 |
| Gastric Cancer 9060397 | 0.0 |
| Stomach Margin 9060396 | 0.0 |
| Gastric Cancer 064005 | 0.0 |

TABLE EF
Panel 4D

| Tissue Name | A | B |
|---|---|---|
| Secondary Th1 act | 0.0 | 0.0 |
| Secondary Th2 act | 0.0 | 0.0 |
| Secondary Tr1 act | 0.0 | 0.0 |
| Secondary Th1 rest | 0.0 | 0.0 |
| Secondary Th2 rest | 0.0 | 0.0 |
| Secondary Tr1 rest | 0.0 | 0.0 |
| Primary Th1 act | 0.0 | 0.0 |
| Primary Th2 act | 0.0 | 0.0 |
| Primary Tr1 act | 1.8 | 0.0 |
| Primary Th1 rest | 0.0 | 0.0 |
| Primary Th2 rest | 0.0 | 0.0 |
| Primary Tr1 rest | 0.0 | 0.0 |
| CD45RA CD4 lymphocyte act | 0.0 | 0.0 |
| CD45RO CD4 lymphocyte act | 0.0 | 0.0 |
| CD8 lymphocyte act | 0.0 | 0.0 |
| Secondary CD8 lymphocyte rest | 0.0 | 0.0 |
| Secondary CD8 lymphocyte act | 0.0 | 0.0 |
| CD4 lymphocyte none | 0.0 | 0.0 |
| 2ry Th1/Th2/Tr1_anti-CD95 CH11 | 0.0 | 0.0 |
| LAK cells rest | 0.0 | 0.0 |
| LAK cells IL-2 | 0.0 | 0.0 |
| LAK cells IL-2 + IL-12 | 0.0 | 0.0 |
| LAK cells IL-2 + IFN gamma | 17.3 | 0.0 |
| LAK cells IL-2 + IL-18 | 0.0 | 0.0 |
| LAK cells PMA/ionomycin | 0.0 | 0.0 |
| NK Cells IL-2 rest | 0.0 | 0.0 |
| Two Way MLR 3 day | 0.0 | 0.0 |
| Two Way MLR 5 day | 17.1 | 0.0 |
| Two Way MLR 7 day | 0.0 | 0.0 |
| PBMC rest | 0.0 | 0.0 |
| PBMC PWM | 0.0 | 0.0 |
| PBMC PHA-L | 0.0 | 0.0 |
| Ramos (B cell) none | 0.0 | 0.0 |
| Ramos (B cell) ionomycin | 0.0 | 0.0 |
| B lymphocytes PWM | 0.0 | 0.0 |
| B lymphocytes CD40L and IL-4 | 0.0 | 0.0 |
| EOL-1 dbcAMP | 0.0 | 0.0 |
| EOL-1 dbcAMP PMA/ionomycin | 0.0 | 0.0 |
| Dendritic cells none | 0.0 | 0.0 |
| Dendritic cells LPS | 2.9 | 0.0 |
| Dendritic cells anti-CD40 | 0.0 | 0.0 |
| Monocytes rest | 0.0 | 0.0 |
| Monocytes LPS | 0.0 | 0.0 |
| Macrophages rest | 8.2 | 0.0 |
| Macrophages LPS | 0.0 | 0.0 |
| HUVEC none | 0.0 | 0.0 |
| HUVEC starved | 1.8 | 0.0 |
| HUVEC IL-1beta | 0.0 | 0.0 |
| HUVEC IFN gamma | 0.0 | 0.0 |
| HUVEC TNF alpha + IFN gamma | 11.6 | 0.0 |
| HUVEC TNF alpha + IL4 | 0.0 | 0.0 |
| HUVEC IL-11 | 8.7 | 0.0 |
| Lung Microvascular EC none | 0.0 | 0.0 |
| Lung Microvascular EC TNFalpha + IL-1beta | 0.0 | 0.0 |
| Microvascular Dermal EC none | 0.0 | 0.0 |
| Microsvasular Dermal EC TNFalpha + IL-1beta | 0.0 | 0.0 |
| Bronchial epithelium TNFalpha + IL1beta | 0.0 | 0.0 |
| Small airway epithelium none | 0.0 | 0.0 |
| Small airway epithelium TNFalpha + IL-1beta | 0.0 | 0.0 |
| Coronery artery SMC rest | 0.0 | 0.0 |
| Coronery artery SMC TNFalpha + ILlbeta | 0.0 | 0.0 |
| Astrocytes rest | 0.0 | 0.0 |
| Astrocytes TNFalpha + IL-1beta | 0.0 | 0.0 |
| KU-812 (Basophil) rest | 0.0 | 25.3 |
| KU-812 (Basophil) PMA/ionomycin | 0.0 | 0.0 |
| CCD1106 (Keratinocytes) none | 0.0 | 0.0 |
| CCD1106 (Keratinocytes) TNFalpha + IL-1beta | 0.0 | 0.0 |
| Liver cirrhosis | 0.0 | 0.0 |
| Lupus kidney | 0.0 | 21.9 |
| NCI-H292 none | 0.0 | 0.0 |
| NCI-H292 IL-4 | 0.0 | 0.0 |
| NCI-H292 IL-9 | 0.0 | 0.0 |
| NCI-H292 IL-13 | 0.0 | 0.0 |
| NCI-H292 IFN gamma | 0.0 | 0.0 |
| HPAEC none | 0.0 | 0.0 |
| HPAEC TNFalpha + IL-1beta | 1.3 | 0.0 |
| Lung fibroblast none | 0.0 | 0.0 |
| Lung fibroblast TNF alpha + IL-1beta | 0.0 | 0.0 |
| Lung fibroblast IL-4 | 0.0 | 0.0 |
| Lung fibroblast IL-9 | 0.0 | 0.0 |
| Lung fibroblast IL-13 | 0.0 | 0.0 |
| Lung fibroblast IFN gamma | 0.0 | 0.0 |
| Dermal fibroblast CCD1070 rest | 0.0 | 0.0 |
| Dermal fibroblast CCD1070 TNF alpha | 0.0 | 0.0 |
| Dermal fibroblast CCD1070 IL-1beta | 0.0 | 0.0 |
| Dermal fibroblast IFN gamma | 0.0 | 0.0 |
| Dermal fibroblast IL-4 | 0.0 | 0.0 |
| IBD Colitis 2 | 0.0 | 0.0 |
| IBD Crohn's | 0.0 | 0.0 |
| Colon | 100.0 | 12.7 |
| Lung | 72.2 | 0.0 |
| Thymus | 47.3 | 100.0 |
| Kidney | 0.0 | 0.0 |

Column A - Rel. Exp. (%) Ag2262, Run 150981162
Column B - Rel. Exp. (%) Ag2316, Run 164037437

Ardais Kidney 1.0 Summary: Ag2262 Highest expression of the CG50709-03 and CG50709-05 genes was detected in a normal kidney sample (CT=27.6). In many cases, expression of these genes was higher in normal adjacent kidney samples relative to the tumors. The results from Panel 1.3D indicate that these genes were also more highly expressed in fetal as compared to adult kidney. This expression profile suggests that the function of these genes is to drive and/or maintain differentiation of kidney epithelium, since loss of differentiation is a hallmark of kidney cancer. Gene or protein expression levels are useful to distinguish normal kidney from kidney cancer. Therapeutic modulation of the activity of these genes or their protein products using nucleic acid, protein, antibody, or small molecule drugs is useful in the treatment of kidney cancer.
Panel 1.3D Summary: Ag2262 Significant expression of these genes was seen mainly in spleen and fetal kidney (CTs=30-32) with upregulated expression in fetal relative to adult kidney. Please see Ardias Kidney v1.0 panel for further discussion of these genes.
Panel 2D Summary: Ag2262 Expression of these genes was highest in a sample derived from normal kidney tissue (CT=32.6) and was generally higher in normal kidney tissue relative to adjacent malignant tissue. This expression profile is in agreement with that seen in the Ardias kidney v1.0 panel. Therapeutic modulation of these genes or their protein products using nucleic acid, protein, antibody or small molecule drugs that increase the activity of these genes is useful in the treatment of kidney cancers.
Panel 4D Summary: Ag2316 Significant expression of these genes was seen exclusively in thymus (CT=33). These genes encode variants of a Wnt14B-like protein; other members of this protein family are known to regulate cell differentiation. The encoded Wnt 14-like proteins may play an important role in T cell development. Therapeutic modulation of the activity of these genes or their protein products is useful to modulate immune function (T cell development) and for organ transplant, AIDS treatment or post chemotherapy immune reconstitiution.

Ag 2262 The Wnt 14B variant recognized by this probe-primer set was significantly expressed in colon, lung and thymus (CT=33-34.7). This gene may play an important role in the normal homeostasis of these tissues. Therapeutic modulation of the activity of this gene or its protein product is useful in maintaining or restoring normal function to these organs during inflammation.

F. CG53054-02: Wnt-14 Protein Precursor

Expression of gene CG53054-02 was assessed using the primer-probe sets Ag2261 and Ag3035, described in Tables FA and FB. Results of the RTQ-PCR runs are shown in Tables FC, FD, FE, FF, FG, FH, FI and FJ.

TABLE FA
Probe Name Ag2261

| | | | Start | SEQ ID |
|---|---|---|---|---|
| Primers | Sequences | Length | Position | No |
| Forward | 5′-ggatgactcgcctagct | 20 | 882 | 301 |
| | tct-3′ | | | |
| Probe | TET-5′-gccgtaggtgccac | 23 | 935 | 302 |
| | cgtgagaag-3′-TAMRA | | | |
| Reverse | 5′-agcagatgctctcgca | 19 | 958 | 303 |
| | gtt-3′ | | | |

TABLE FB
Probe Name Ag3035

| | | | Start | SEQ ID |
|---|---|---|---|---|
| Primers | Sequences | Length | Position | No |
| Forward | 5′-acagcagcaagttcgtc | 20 | 527 | 304 |
| | aag-3′ | | | |
| Probe | TET-5′-agacggtcaagcaa | 25 | 559 | 305 |
| | ggatctgcgag-3′-TAMRA | | | |
| Reverse | 5′-cacgaggttgttgtgga | 20 | 593 | 306 |
| | agt-3′ | | | |

TABLE FC
AI comprehensive panel v1.0
Column A - Rel. Exp. (%) Ag3035, Run 311087483

| Tissue Name | A |
|---|---|
| 110967 COPD-F | 13.1 |
| 110980 COPD-F | 44.1 |
| 110968 COPD-M | 32.3 |
| 110977 COPD-M | 100.0 |
| 110989 Emphysema-F | 48.0 |
| 110992 Emphysema-F | 28.7 |
| 110993 Emphysema-F | 17.9 |
| 110994 Emphysema-F | 7.4 |
| 110995 Emphysema-F | 50.3 |
| 110996 Emphysema-F | 17.3 |
| 110997 Asthma-M | 1.6 |
| 111001 Asthma-F | 13.1 |
| 111002 Asthma-F | 46.7 |
| 111003 Atopic Asthma-F | 25.3 |
| 111004 Atopic Asthma-F | 44.4 |
| 111005 Atopic Asthma-F | 22.4 |
| 111006 Atopic Asthma-F | 6.9 |
| 111417 Allergy-M | 22.1 |
| 112347 Allergy-M | 2.2 |
| 112349 Normal Lung-F | 0.9 |
| 112357 Normal Lung-F | 76.8 |
| 112354 Normal Lung-M | 11.3 |
| 112374 Crohns-F | 30.1 |
| 112389 Match Control Crohns-F | 20.6 |
| 112375 Crohns-F | 29.9 |
| 112732 Match Control Crohns-F | 14.3 |
| 112725 Crohns-M | 6.4 |
| 112387 Match Control Crohns-M | 18.8 |
| 112378 Crohns-M | 1.6 |
| 112390 Match Control Crohns-M | 25.9 |
| 112726 Crohns-M | 14.1 |
| 112731 Match Control Crohns-M | 25.9 |
| 112380 Ulcer Col-F | 33.0 |
| 112734 Match Control Ulcer Col-F | 19.2 |
| 112384 Ulcer Col-F | 21.8 |
| 112737 Match Control Ulcer Col-F | 3.6 |
| 112386 Ulcer Col-F | 10.8 |
| 112738 Match Control Ulcer Col-F | 25.5 |
| 112381 Ulcer Col-M | 0.2 |
| 112735 Match Control Ulcer Col-M | 1.9 |
| 112382 Ulcer Col-M | 14.4 |
| 112394 Match Control Ulcer Col-M | 3.1 |
| 112383 Ulcer Col-M | 31.6 |
| 112736 Match Control Ulcer Col-M | 11.3 |
| 112423 Psoriasis-F | 7.1 |
| 112427 Match Control Psoriasis-F | 84.1 |
| 112418 Psoriasis-M | 11.5 |
| 112723 Match Control Psoriasis-M | 2.6 |
| 112419 Psoriasis-M | 17.3 |
| 112424 Match Control Psoriasis-M | 9.9 |
| 112420 Psoriasis-M | 45.1 |
| 112425 Match Control Psoriasis-M | 36.1 |
| 104689 (MF) OA Bone-Backus | 16.6 |
| 104690 (MF) Adj “Normal” Bone-Backus | 10.2 |
| 104691 (MF) OA Synovium-Backus | 27.9 |
| 104692 (BA) OA Cartilage-Backus | 0.0 |
| 104694 (BA) OA Bone-Backus | 21.3 |
| 104695 (BA) Adj “Normal” Bone-Backus | 8.1 |
| 104696 (BA) OA Synovium-Backus | 22.1 |
| 104700 (SS) OA Bone-Backus | 6.0 |
| 104701 (SS) Adj “Normal” Bone-Backus | 12.4 |
| 104702 (SS) OA Synovium-Backus | 39.5 |
| 117093 OA Cartilage Rep7 | 21.3 |
| 112672 OA Bone5 | 17.9 |
| 112673 OA Synovium5 | 10.1 |
| 112674 OA Synovial Fluid cells5 | 7.6 |
| 117100 OA Cartilage Rep14 | 12.8 |
| 112756 OA Bone9 | 24.0 |
| 112757 OA Synovium9 | 49.3 |
| 112758 OA Synovial Fluid Cells9 | 5.7 |
| 117125 RA Cartilage Rep2 | 17.3 |
| 113492 Bone2 RA | 25.5 |
| 113493 Synovium2 RA | 6.3 |
| 113494 Syn Fluid Cells RA | 16.8 |
| 113499 Cartilage4 RA | 13.1 |
| 113500 Bone4 RA | 21.8 |
| 113501 Synovium4 RA | 13.9 |
| 113502 Syn Fluid Cells4 RA | 6.8 |
| 113495 Cartilage3 RA | 14.4 |
| 113496 Bone3 RA | 13.9 |
| 113497 Synovium3 RA | 10.0 |
| 113498 Syn Fluid Cells3 RA | 23.0 |
| 117106 Normal Cartilage Rep20 | 15.5 |
| 113663 Bone3 Normal | 0.0 |
| 113664 Synovium3 Normal | 0.0 |
| 113665 Syn Fluid Cells3 Normal | 0.1 |
| 117107 Normal Cartilage Rep22 | 3.6 |
| 113667 Bone4 Normal | 18.3 |
| 113668 Synovium4 Normal | 16.4 |
| 113669 Syn Fluid Cells4 Normal | 43.8 |

TABLE FD
Oncology cell line screening panel v3.2
Column A - Rel. Exp. (%) Ag3035, Run 259180377

| Tissue Name | A |
|---|---|
| 94905_Daoy_Medulloblastoma/Cerebellum_sscDNA | 0.2 |
| 94906_TE671_Medulloblastom/Cerebellum_sscDNA | 0.5 |
| 94907_D283 | 0.2 |
| Med_Medulloblastoma/Cerebellum_sscDNA | |
| 94908_PFSK-1_Primitive | 0.0 |
| Neuroectodermal/Cerebellum_sscDNA | |
| 94909_XF-498_CNS_sscDNA | 0.2 |
| 94910_SNB-78_CNS/glioma_sscDNA | 0.5 |
| 94911_SF-268_CNS/glioblastoma_sscDNA | 0.3 |
| 94912_T98G_Glioblastoma_sscDNA | 0.5 |
| 96776_SK-N-SH_Neuroblastoma | 0.0 |
| (metastasis)_sscDNA | |
| 94913_SF-295_CNS/glioblastoma_sscDNA | 0.6 |
| 132565_NT2 pool_sscDNA | 3.9 |
| 94914_Cerebellum_sscDNA | 0.7 |
| 96777_Cerebellum_sscDNA | 0.8 |
| 94916_NCI-H292_Mucoepidermoid lung | 11.7 |
| carcinoma_sscDNA | |
| 94917_DMS-114_Small cell lung | 0.3 |
| cancer_sscDNA | |
| 94918_DMS-79_Small cell lung | 100.0 |
| cancer/neuroendocrine_sscDNA | |
| 94919_NCI-H146_Small cell lung | 16.4 |
| cancer/neuroendocrine_sscDNA | |
| 94920_NCI-H526_Small cell lung | 0.0 |
| cancer/neuroendocrine_sscDNA | |
| 94921_NCI-N417_Small cell lung | 0.0 |
| cancer/neuroendocrine_sscDNA | |
| 94923_NCI-H82_Small cell lung | 0.4 |
| cancer/neuroendocrine_sscDNA | |
| 94924_NCI-H157_Squamous cell lung cancer | 2.4 |
| (metastasis)_sscDNA | |
| 94925_NCI-H1155_Large cell lung | 15.5 |
| cancer/neuroendocrine_sscDNA | |
| 94926_NCI-H1299_Large cell lung | 3.4 |
| cancer/neuroendocrine_sscDNA | |
| 94927_NCI-H727_Lung carcinoid_sscDNA | 9.5 |
| 94928_NCI-UMC-11_Lung carcinoid_sscDNA | 20.2 |
| 94929_LX-1_Small cell lung cancer_sscDNA | 0.0 |
| 94930_Colo-205_Colon cancer_sscDNA | 0.0 |
| 94931_KM12_Colon cancer_sscDNA | 0.0 |
| 94932_KM20L2_Colon cancer_sscDNA | 0.0 |
| 94933_NCI-H716_Colon cancer_sscDNA | 4.3 |
| 94935_SW-48_Colon adenocarcinoma_sscDNA | 0.0 |
| 94936_SW1116_Colon | 1.2 |
| adenocarcinoma_sscDNA | |
| 94937_LS 174T_Colon | 0.6 |
| adenocarcinoma_sscDNA | |
| 94938_SW-948_Colon | 0.0 |
| adenocarcinoma_sscDNA | |
| 94939_SW-480_Colon | 0.0 |
| adenocarcinoma_sscDNA | |
| 94940_NCI-SNU-5_Gastric carcinoma_sscDNA | 6.9 |
| 112197_KATO III_Stomach_sscDNA | 0.3 |
| 94943_NCI-SNU-16_Gastric | 0.6 |
| carcinoma_sscDNA | |
| 94944_NCI-SNU-1_Gastric carcinoma_sscDNA | 2.9 |
| 94946_RF-1_Gastric adenocarcinoma_sscDNA | 0.0 |
| 94947_RF-48_Gastric | 0.0 |
| adenocarcinoma_sscDNA | |
| 96778_MKN-45_Gastric carcinoma_sscDNA | 0.6 |
| 94949_NCI-N87_Gastric carcinoma_sscDNA | 3.9 |
| 94951_OVCAR-5_Ovarian carcinoma_sscDNA | 1.3 |
| 94952_RL95-2_Uterine carcinoma_sscDNA | 3.0 |
| 94953_HelaS3_Cervical | 1.7 |
| adenocarcinoma_sscDNA | |
| 94954_Ca Ski_Cervical epidermoid | 8.4 |
| carcinoma (metastasis)_sscDNA | |
| 94955_ES-2_Ovarian clear cell | 0.0 |
| carcinoma_sscDNA | |
| 94957_Ramos/6 h stim_Stimulated with | 0.0 |
| PMA/ionomycin 6 h_sscDNA | |
| 94958_Ramos/14 h stim_Stimulated | 0.0 |
| with PMA/ionomycin 14 h_sscDNA | |
| 94962_MEG-01_Chronic myelogenous | 0.0 |
| leukemia (megokaryoblast)_sscDNA | |
| 94963_Raji_Burkitt's | 0.0 |
| lymphoma_sscDNA | |
| 94964_Daudi_Burkitt's | 0.0 |
| lymphoma_sscDNA | |
| 94965_U266_B-cell | 0.0 |
| plasmacytoma/myeloma_sscDNA | |
| 94968_CA46_Burkitt's | 0.0 |
| lymphoma_sscDNA | |
| 94970_RL_non-Hodgkin's B-cell | 0.0 |
| lymphoma_sscDNA | |
| 94972_JM1_pre-B-cell | 1.1 |
| lymphoma/leukemia_sscDNA | |
| 94973_Jurkat_T cell leukemia_sscDNA | 0.0 |
| 94974_TF-1_Erythroleukemia_sscDNA | 0.0 |
| 94975_HUT 78_T-cell | 0.1 |
| lymphoma_sscDNA | |
| 94977_U937_Histiocytic | 0.0 |
| lymphoma_sscDNA | |
| 94980_KU-812_Myelogenous | 1.3 |
| leukemia_sscDNA | |
| 94981_769-P_Clear cell renal | 1.9 |
| carcinoma_sscDNA | |
| 94983_Caki-2_Clear cell renal | 1.8 |
| carcinoma_sscDNA | |
| 94984_SW 839_Clear cell renal | 0.8 |
| carcinoma_sscDNA | |
| 94986_G401_Wilms' tumor_sscDNA | 0.4 |
| 126768_293 cells_sscDNA | 1.9 |
| 94987_Hs766T_Pancreatic carcinoma | 2.1 |
| (LN metastasis)_sscDNA | |
| 94988_CAPAN-1_Pancreatic | 3.2 |
| adenocarcinoma (liver | |
| metastasis)_sscDNA | |
| 94989_SU86.86_Pancreatic carcinoma | 1.6 |
| (liver metastasis)_sscDNA | |
| 94990_BxPC-3_Pancreatic | 3.0 |
| adenocarcinoma_sscDNA | |
| 94991_HPAC_Pancreatic | 0.9 |
| adenocarcinoma_sscDNA | |
| 94992_MIA PaCa-2_Pancreatic | 1.1 |
| carcinoma_sscDNA | |
| 94993_CFPAC-1_Pancreatic ductal | 0.2 |
| adenocarcinoma_sscDNA | |
| 94994_PANC-1_Pancreatic epithelioid | 22.8 |
| ductal carcinoma_sscDNA | |
| 94996_T24_Bladder carcinma | 0.2 |
| (transitional cell)_sscDNA | |
| 94997_5637_Bladder | 0.5 |
| carcinoma_sscDNA | |
| 94998_HT-1197_Bladder | 12.5 |
| carcinoma_sscDNA | |
| 94999_UM-UC-3_Bladder carcinma | 0.0 |
| (transitional cell)_sscDNA | |
| 95000_A204_Rhabdomyosarcoma_{—} | 0.0 |
| sscDNA | |
| 95001_HT-1080_Fibrosarcoma_sscDNA | 1.6 |
| 95002_MG-63_Osteosarcoma | 14.7 |
| (bone)_sscDNA | |
| 95003_SK-LMS-1_Leiomyosarcoma | 0.0 |
| (vulva)_sscDNA | |
| 95004_SJRH30_Rhabdomyosarcoma | 1.3 |
| (met to bone marrow)_sscDNA | |
| 95005_A431_Epidermoid | 5.9 |
| carcinoma_sscDNA | |
| 95007_WM266-4_Melanoma_sscDNA | 0.0 |
| 112195_DU 145_Prostate_sscDNA | 1.1 |
| 95012_MDA-MB-468_Breast | 2.2 |
| adenocarcinoma_sscDNA | |
| 112196_SSC-4_Tongue_sscDNA | 2.6 |
| 112194_SSC-9_Tongue_sscDNA | 3.3 |
| 112191_SSC-15_Tongue_sscDNA | 1.8 |
| 95017_CAL 27_Squamous cell | 1.2 |
| carcinoma of tongue_sscDNA | |

TABLE FE
Panel 1.3D

| Tissue Name | A | B | C |
|---|---|---|---|
| Liver adenocarcinoma | 22.4 | 19.6 | 71.2 |
| Pancreas | 3.9 | 2.5 | 2.8 |
| Pancreatic ca. CAPAN 2 | 5.3 | 3.5 | 9.5 |
| Adrenal gland | 2.1 | 0.6 | 2.0 |
| Thyroid | 7.0 | 9.8 | 3.9 |
| Salivary gland | 1.9 | 2.1 | 4.2 |
| Pituitary gland | 1.0 | 2.2 | 6.7 |
| Brain (fetal) | 6.8 | 4.9 | 10.8 |
| Brain (whole) | 4.8 | 3.0 | 1.4 |
| Brain (amygdala) | 4.6 | 5.3 | 1.5 |
| Brain (cerebellum) | 1.6 | 1.6 | 2.0 |
| Brain (hippocampus) | 7.5 | 11.3 | 0.6 |
| Brain (substantia nigra) | 1.2 | 2.6 | 1.3 |
| Brain (thalamus) | 2.5 | 1.7 | 2.6 |
| Cerebral Cortex | 0.0 | 0.0 | 5.0 |
| Spinal cord | 1.7 | 2.1 | 2.7 |
| glio/astro U87-MG | 0.0 | 0.0 | 0.0 |
| glio/astro U-118-MG | 55.1 | 50.3 | 42.9 |
| astrocytoma SW1783 | 0.0 | 7.5 | 0.0 |
| neuro*; met SK-N-AS | 0.0 | 0.0 | 0.7 |
| astrocytoma SF-539 | 1.9 | 4.7 | 9.9 |
| astrocytoma SNB-75 | 2.0 | 4.9 | 6.9 |
| glioma SNB-19 | 6.7 | 2.4 | 3.7 |
| glioma U251 | 2.1 | 4.5 | 6.8 |
| glioma SF-295 | 10.0 | 0.6 | 4.6 |
| Heart (fetal) | 11.1 | 9.9 | 38.2 |
| Heart | 4.9 | 6.0 | 15.2 |
| Skeletal muscle (fetal) | 100.0 | 100.0 | 85.3 |
| Skeletal muscle | 5.5 | 8.4 | 39.8 |
| Bone marrow | 0.0 | 0.0 | 0.7 |
| Thymus | 10.0 | 3.9 | 6.4 |
| Spleen | 3.8 | 4.2 | 1.6 |
| Lymph node | 5.0 | 1.1 | 1.4 |
| Colorectal | 3.4 | 5.4 | 6.8 |
| Stomach | 6.0 | 15.4 | 3.1 |
| Small intestine | 15.9 | 18.7 | 2.3 |
| Colon ca. SW480 | 24.3 | 15.3 | 11.6 |
| Colon ca.* SW620(SW480 met) | 0.0 | 0.0 | 2.1 |
| Colon ca. HT29 | 0.0 | 0.0 | 0.0 |
| Colon ca. HCT-116 | 3.8 | 0.6 | 3.3 |
| Colon ca. CaCo-2 | 0.0 | 0.8 | 0.3 |
| Colon ca. tissue(ODO3866) | 2.3 | 0.0 | 1.6 |
| Colon ca. HCC-2998 | 0.0 | 0.0 | 1.6 |
| Gastric ca.* (liver met) | 16.7 | 14.9 | 15.3 |
| NCI-N87 | | | |
| Bladder | 1.6 | 3.2 | 3.0 |
| Trachea | 24.3 | 33.7 | 5.7 |
| Kidney | 0.0 | 0.0 | 0.0 |
| Kidney (fetal) | 2.1 | 0.0 | 2.7 |
| Renal ca. 786-0 | 0.0 | 0.0 | 0.0 |
| Renal ca. A498 | 10.2 | 5.3 | 9.2 |
| Renal ca. RXF 393 | 0.0 | 0.0 | 0.0 |
| Renal ca. ACHN | 0.0 | 2.2 | 0.0 |
| Renal ca. UO-31 | 0.0 | 0.0 | 0.0 |
| Renal ca. TK-10 | 0.0 | 0.0 | 0.0 |
| Liver | 0.0 | 0.0 | 0.0 |
| Liver (fetal) | 7.6 | 0.0 | 0.0 |
| Liver ca. (hepatoblast) HepG2 | 0.0 | 0.0 | 0.0 |
| Lung | 14.3 | 15.8 | 9.2 |
| Lung (fetal) | 15.1 | 15.4 | 7.4 |
| Lung ca. (small cell) LX-1 | 1.6 | 0.0 | 0.0 |
| Lung ca. (small cell) NCI-H69 | 29.5 | 19.1 | 31.2 |
| Lung ca. (s. cell var.) SHP-77 | 11.0 | 5.1 | 37.4 |
| Lung ca. (large cell) NCI-H460 | 0.0 | 0.0 | 0.0 |
| Lung ca. (non-sm. cell) A549 | 0.0 | 1.2 | 1.6 |
| Lung ca. (non-s. cell) NCI-H23 | 0.0 | 1.3 | 0.8 |
| Lung ca. (non-s. cell) HOP-62 | 0.0 | 1.7 | 0.0 |
| Lung ca. (non-s. cl) NCI-H522 | 8.0 | 8.3 | 7.3 |
| Lung ca. (squam.) SW 900 | 4.0 | 0.0 | 1.8 |
| Lung ca. (squam.) NCI-H596 | 15.8 | 10.2 | 58.2 |
| Mammary gland | 7.2 | 4.1 | 4.4 |
| Breast ca.* (pl. ef) MCF-7 | 1.7 | 3.4 | 7.3 |
| Breast ca.* (pl. ef) MDA-MB-231 | 23.2 | 19.6 | 19.2 |
| Breast ca.* (pl. ef) T47D | 4.3 | 5.8 | 21.8 |
| Breast ca. BT-549 | 0.0 | 4.2 | 2.2 |
| Breast ca. MDA-N | 0.0 | 0.0 | 0.0 |
| Ovary | 3.6 | 3.1 | 8.1 |
| Ovarian ca. OVCAR-3 | 1.1 | 1.0 | 5.6 |
| Ovarian ca. OVCAR-4 | 0.0 | 0.0 | 0.7 |
| Ovarian ca. OVCAR-5 | 0.0 | 0.0 | 11.5 |
| Ovarian ca. OVCAR-8 | 1.3 | 4.3 | 4.1 |
| Ovarian ca. IGROV-1 | 0.0 | 0.0 | 8.1 |
| Ovarian ca.* (ascites) SK-OV-3 | 7.5 | 16.0 | 100.0 |
| Uterus | 17.8 | 15.1 | 9.9 |
| Placenta | 4.6 | 8.2 | 2.1 |
| Prostate | 3.6 | 5.3 | 0.6 |
| Prostate ca.* (bone met) PC-3 | 1.7 | 1.5 | 6.1 |
| Testis | 21.9 | 14.6 | 1.6 |
| Melanoma Hs688(A).T | 3.1 | 4.7 | 1.4 |
| Melanoma* (met) Hs688(B).T | 0.4 | 1.3 | 0.0 |
| Melanoma UACC-62 | 0.0 | 0.0 | 0.0 |
| Melanoma M14 | 0.0 | 0.0 | 0.0 |
| Melanoma LOX IMVI | 0.0 | 0.0 | 0.0 |
| Melanoma* (met) SK-MEL-5 | 0.0 | 2.0 | 0.7 |
| Adipose | 6.7 | 7.2 | 21.2 |

Column A - Rel. Exp. (%) Ag2261, Run 150631675
Column B - Rel. Exp. (%) Ag2261, Run 152887692
Column C - Rel. Exp. (%) Ag3035, Run 167597764

TABLE FF
Panel 2D

| Tissue Name | A | B |
|---|---|---|
| Normal Colon | 19.1 | 19.8 |
| CC Well to Mod Diff (ODO3866) | 0.0 | 5.8 |
| CC Margin (ODO3866) | 19.5 | 12.5 |
| CC Gr.2 rectosigmoid (ODO3868) | 3.8 | 1.4 |
| CC Margin (ODO3868) | 2.6 | 5.1 |
| CC Mod Diff (ODO3920) | 6.0 | 2.9 |
| CC Margin (ODO3920) | 23.8 | 6.4 |
| CC Gr.2 ascend colon (ODO3921) | 9.3 | 2.2 |
| CC Margin (ODO3921) | 16.8 | 11.7 |
| CC from Partial Hepatectomy | 2.4 | 0.0 |
| (ODO4309) Mets | | |
| Liver Margin (ODO4309) | 2.6 | 0.0 |
| Colon mets to lung (OD04451-01) | 7.9 | 4.5 |
| Lung Margin (OD04451-02) | 11.3 | 12.9 |
| Normal Prostate 6546-1 | 6.3 | 2.6 |
| Prostate Cancer (OD04410) | 17.8 | 7.3 |
| Prostate Margin (OD04410) | 10.7 | 7.4 |
| Prostate Cancer (OD04720-01) | 4.7 | 4.4 |
| Prostate Margin (OD04720-02) | 13.9 | 5.6 |
| Normal Lung 061010 | 36.6 | 14.3 |
| Lung Met to Muscle (ODO4286) | 1.0 | 0.0 |
| Muscle Margin (ODO4286) | 31.0 | 38.2 |
| Lung Malignant Cancer (OD03126) | 81.8 | 100.0 |
| Lung Margin (OD03126) | 35.8 | 18.2 |
| Lung Cancer (OD04404) | 57.0 | 39.5 |
| Lung Margin (OD04404) | 9.4 | 11.8 |
| Lung Cancer (OD04565) | 37.1 | 42.0 |
| Lung Margin (OD04565) | 22.7 | 9.3 |
| Lung Cancer (OD04237-01) | 5.3 | 6.4 |
| Lung Margin (OD04237-02) | 78.5 | 32.8 |
| Ocular Mel Met to Liver (ODO4310) | 0.0 | 0.0 |
| Liver Margin (ODO4310) | 2.4 | 0.0 |
| Melanoma Mets to Lung (OD04321) | 13.0 | 0.0 |
| Lung Margin (OD04321) | 96.6 | 50.0 |
| Normal Kidney | 0.0 | 0.0 |
| Kidney Ca, Nuclear grade 2 (OD04338) | 0.0 | 0.0 |
| Kidney Margin (OD04338) | 4.0 | 4.6 |
| Kidney Ca Nuclear grade 1/2 (OD04339) | 0.0 | 3.3 |
| Kidney Margin (OD04339) | 18.7 | 0.0 |
| Kidney Ca, Clear cell type (OD04340) | 8.8 | 11.7 |
| Kidney Margin (OD04340) | 0.0 | 2.0 |
| Kidney Ca, Nuclear grade 3 (OD04348) | 3.5 | 4.0 |
| Kidney Margin (OD04348) | 2.0 | 1.7 |
| Kidney Cancer (OD04622-01) | 9.3 | 0.0 |
| Kidney Margin (OD04622-03) | 0.0 | 6.3 |
| Kidney Cancer (OD04450-01) | 0.0 | 0.0 |
| Kidney Margin (OD04450-03) | 0.0 | 0.0 |
| Kidney Cancer 8120607 | 0.0 | 0.7 |
| Kidney Margin 8120608 | 2.4 | 0.0 |
| Kidney Cancer 8120613 | 14.6 | 7.3 |
| Kidney Margin 8120614 | 4.8 | 1.5 |
| Kidney Cancer 9010320 | 0.0 | 0.0 |
| Kidney Margin 9010321 | 0.0 | 0.0 |
| Normal Uterus | 9.7 | 2.8 |
| Uterus Cancer 064011 | 85.9 | 41.5 |
| Normal Thyroid | 15.2 | 7.3 |
| Thyroid Cancer 064010 | 0.0 | 3.0 |
| Thyroid Cancer A302152 | 1.9 | 1.2 |
| Thyroid Margin A302153 | 2.6 | 2.8 |
| Normal Breast | 16.2 | 2.7 |
| Breast Cancer (OD04566) | 78.5 | 29.7 |
| Breast Cancer (OD04590-01) | 37.6 | 23.8 |
| Breast Cancer Mets (OD04590-03) | 100.0 | 24.5 |
| Breast Cancer Metastasis (OD04655-05) | 94.0 | 45.4 |
| Breast Cancer 064006 | 25.7 | 24.8 |
| Breast Cancer 1024 | 23.2 | 7.1 |
| Breast Cancer 9100266 | 33.0 | 7.5 |
| Breast Margin 9100265 | 7.6 | 7.6 |
| Breast Cancer A209073 | 13.9 | 0.9 |
| Breast Margin A209073 | 2.5 | 0.0 |
| Normal Liver | 0.0 | 0.0 |
| Liver Cancer 064003 | 0.0 | 0.0 |
| Liver Cancer 1025 | 4.8 | 1.7 |
| Liver Cancer 1026 | 7.1 | 0.0 |
| Liver Cancer 6004-T | 4.8 | 0.0 |
| Liver Tissue 6004-N | 4.4 | 1.8 |
| Liver Cancer 6005-T | 0.0 | 6.0 |
| Liver Tissue 6005-N | 0.0 | 1.8 |
| Normal Bladder | 2.4 | 3.0 |
| Bladder Cancer 1023 | 8.5 | 4.9 |
| Bladder Cancer A302173 | 17.0 | 11.8 |
| Bladder Cancer (OD04718-01) | 10.0 | 5.7 |
| Bladder Normal Adjacent (OD04718-03) | 19.3 | 27.5 |
| Normal Ovary | 13.6 | 12.4 |
| Ovarian Cancer 064008 | 37.9 | 2.1 |
| Ovarian Cancer (OD04768-07) | 18.4 | 3.7 |
| Ovary Margin (OD04768-08) | 28.3 | 12.2 |
| Normal Stomach | 48.3 | 17.3 |
| Gastric Cancer 9060358 | 0.0 | 0.0 |
| Stomach Margin 9060359 | 9.9 | 3.0 |
| Gastric Cancer 9060395 | 20.7 | 10.4 |
| Stomach Margin 9060394 | 10.0 | 12.2 |
| Gastric Cancer 9060397 | 8.7 | 1.5 |
| Stomach Margin 9060396 | 7.5 | 6.2 |
| Gastric Cancer 064005 | 10.7 | 4.8 |

Column A - Rel. Exp. (%) Ag2261, Run 150811744
Column B - Rel. Exp. (%) Ag2261, Run 152887693

TABLE FG
Panel 4.1D

| Tissue Name | A | B |
|---|---|---|
| Secondary Th1 act | 0.0 | 0.0 |
| Secondary Th2 act | 0.0 | 0.0 |
| Secondary Tr1 act | 0.0 | 0.0 |
| Secondary Th1 rest | 0.0 | 0.0 |
| Secondary Th2 rest | 0.0 | 2.0 |
| Secondary Tr1 rest | 0.0 | 0.0 |
| Primary Th1 act | 0.0 | 0.0 |
| Primary Th2 act | 0.0 | 0.0 |
| Primary Tr1 act | 0.0 | 0.0 |
| Primary Th1 rest | 0.0 | 0.0 |
| Primary Th2 rest | 0.0 | 0.0 |
| Primary Tr1 rest | 0.0 | 0.0 |
| CD45RA CD4 lymphocyte act | 0.0 | 2.3 |
| CD45RO CD4 lymphocyte act | 0.0 | 0.0 |
| CD8 lymphocyte act | 0.0 | 0.0 |
| Secondary CD8 lymphocyte rest | 0.0 | 0.0 |
| Secondary CD8 lymphocyte act | 0.0 | 0.0 |
| CD4 lymphocyte none | 0.0 | 0.0 |
| 2ry Th1/Th2/Tr1_anti-CD95 | 0.0 | 0.0 |
| CH11 | | |
| LAK cells rest | 0.0 | 4.7 |
| LAK cells IL-2 | 0.0 | 0.0 |
| LAK cells IL-2 + IL-12 | 0.0 | 0.0 |
| LAK cells IL-2 + IFN gamma | 0.0 | 0.0 |
| LAK cells IL-2 + IL-18 | 0.0 | 3.0 |
| LAK cells PMA/ionomycin | 11.0 | 42.0 |
| NK Cells IL-2 rest | 0.0 | 5.2 |
| Two Way MLR 3 day | 0.0 | 0.0 |
| Two Way MLR 5 day | 0.0 | 0.0 |
| Two Way MLR 7 day | 0.0 | 0.0 |
| PBMC rest | 0.0 | 0.0 |
| PBMC PWM | 0.5 | 0.0 |
| PBMC PHA-L | 0.4 | 0.0 |
| Ramos (B cell) none | 0.0 | 0.0 |
| Ramos (B cell) ionomycin | 0.0 | 0.0 |
| B lymphocytes PWM | 0.0 | 0.0 |
| B lymphocytes CD40L and IL-4 | 0.0 | 0.0 |
| EOL-1 dbcAMP | 0.0 | 2.7 |
| EOL-1 dbcAMP PMA/ionomycin | 1.0 | 1.7 |
| Dendritic cells none | 0.0 | 0.0 |
| Dendritic cells LPS | 0.0 | 0.0 |
| Dendritic cells anti-CD40 | 0.0 | 0.0 |
| Monocytes rest | 0.0 | 0.0 |
| Monocytes LPS | 0.6 | 0.0 |
| Macrophages rest | 0.0 | 0.0 |
| Macrophages LPS | 0.0 | 0.0 |
| HUVEC none | 2.4 | 7.6 |
| HUVEC starved | 8.8 | 22.1 |
| HUVEC IL-1beta | 1.7 | 0.0 |
| HUVEC IFN gamma | 0.8 | 11.8 |
| HUVEC TNF alpha + IFN gamma | 0.2 | 0.0 |
| HUVEC TNF alpha + IL4 | 0.6 | 4.5 |
| HUVEC IL-11 | 1.1 | 17.2 |
| Lung Microvascular EC none | 2.7 | 7.2 |
| Lung Microvascular EC TNFalpha 4 + IL-1beta | 0.6 | 0.0 |
| Microvascular Dermal EC none | 3.8 | 0.0 |
| Microsvasular Dermal EC TNFalpha + IL-1beta | 1.2 | 0.0 |
| Bronchial epithelium TNFalpha + IL1beta | 3.7 | 5.0 |
| Small airway epithelium none | 1.9 | 10.7 |
| Small airway epithelium TNFalpha + | 4.0 | 24.5 |
| IL-1beta | | |
| Coronery artery SMC rest | 0.2 | 2.2 |
| Coronery artery SMC TNFalpha + | 0.0 | 0.0 |
| IL-1beta | | |
| Astrocytes rest | 2.4 | 7.9 |
| Astrocytes TNFalpha + IL-1beta | 1.3 | 0.0 |
| KU-812 (Basophil) rest | 0.0 | 2.3 |
| KU-812 (Basophil) PMA/ionomycin | 2.1 | 4.5 |
| CCD1106 (Keratinocytes) none | 22.2 | 100.0 |
| CCD1106 (Keratinocytes) TNFalpha + IL-1beta | 18.8 | 52.5 |
| Liver cirrhosis | 0.7 | 4.2 |
| NCI-H292 none | 0.4 | 0.0 |
| NCI-H292 IL-4 | 1.5 | 0.0 |
| NCI-H292 IL-9 | 2.0 | 8.8 |
| NCI-H292 IL-13 | 1.4 | 17.8 |
| NCI-H292 IFN gamma | 1.5 | 6.9 |
| HPAEC none | 3.1 | 18.7 |
| HPAEC TNF alpha + IL-1beta | 0.5 | 0.0 |
| Lung fibroblast none | 6.2 | 29.9 |
| Lung fibroblast TNF alpha + IL-1beta | 2.1 | 0.0 |
| Lung fibroblast IL-4 | 4.2 | 9.8 |
| Lung fibroblast IL-9 | 8.3 | 25.3 |
| Lung fibroblast IL-13 | 4.0 | 3.9 |
| Lung fibroblast IFN gamma | 8.1 | 59.5 |
| Dermal fibroblast CCD1070 rest | 0.4 | 0.0 |
| Dermal fibroblast CCD1070 TNF alpha | 0.9 | 4.1 |
| Dermal fibroblast CCD1070 IL-1 beta | 2.9 | 0.0 |
| Dermal fibroblast IFN gamma | 5.8 | 41.8 |
| Dermal fibroblast IL-4 | 17.2 | 62.4 |
| Dermal Fibroblasts rest | 4.8 | 12.9 |
| Neutrophils TNFa + LPS | 1.0 | 0.0 |
| Neutrophils rest | 2.2 | 2.3 |
| Colon | 2.6 | 0.0 |
| Lung | 8.8 | 0.0 |
| Thymus | 17.1 | 1.8 |
| Kidney | 100.0 | 0.0 |

Column A - Rel. Exp. (%) Ag3035, Run 190944495
Column B - Rel. Exp. (%) Ag3035, Run 259180379

TABLE FH
Panel 4D

| Tissue Name | A | B |
|---|---|---|
| Secondary Th1 act | 0.0 | 2.1 |
| Secondary Th2 act | 0.0 | 0.0 |
| Secondary Tr1 act | 0.0 | 4.2 |
| Secondary Th1 rest | 0.0 | 0.0 |
| Secondary Th2 rest | 0.0 | 2.3 |
| Secondary Tr1 rest | 0.0 | 0.0 |
| Primary Th1 act | 0.0 | 0.0 |
| Primary Th2 act | 0.0 | 0.0 |
| Primary Tr1 act | 0.0 | 0.0 |
| Primary Th1 rest | 0.0 | 0.0 |
| Primary Th2 rest | 0.0 | 0.0 |
| Primary Tr1 rest | 0.0 | 0.0 |
| CD45RA CD4 lymphocyte act | 0.0 | 0.0 |
| CD45RO CD4 lymphocyte act | 0.0 | 0.0 |
| CD8 lymphocyte act | 0.0 | 0.0 |
| Secondary CD8 lymphocyte rest | 0.0 | 0.7 |
| Secondary CD8 lymphocyte act | 1.6 | 0.0 |
| CD4 lymphocyte none | 0.0 | 0.0 |
| 2ry Th1/Th2/Tr1_anti-CD95 CH11 | 0.0 | 1.4 |
| LAK cells rest | 3.5 | 0.0 |
| LAK cells IL-2 | 0.0 | 0.0 |
| LAK cells IL-2 + IL-12 | 0.0 | 0.0 |
| LAK cells IL-2 + IFN gamma | 0.0 | 4.0 |
| LAK cells IL-2 + IL-18 | 0.0 | 0.0 |
| LAK cells PMA/ionomycin | 26.1 | 50.7 |
| NK Cells IL-2 rest | 0.0 | 0.0 |
| Two Way MLR 3 day | 0.0 | 0.0 |
| Two Way MLR 5 day | 0.0 | 0.0 |
| Two Way MLR 7 day | 0.0 | 0.0 |
| PBMC rest | 0.0 | 0.0 |
| PBMC PWM | 0.0 | 0.0 |
| PBMC PHA-L | 0.0 | 0.0 |
| Ramos (B cell) none | 0.0 | 0.0 |
| Ramos (B cell) ionomycin | 0.0 | 0.0 |
| B lymphocytes PWM | 0.0 | 0.0 |
| B lymphocytes CD40L and IL-4 | 3.1 | 0.0 |
| EOL-1 dbcAMP | 0.0 | 0.0 |
| EOL-1 dbcAMP PMA/ionomycin | 3.5 | 2.7 |
| Dendritic cells none | 0.0 | 0.0 |
| Dendritic cells LPS | 0.0 | 0.0 |
| Dendritic cells anti-CD40 | 0.0 | 0.0 |
| Monocytes rest | 0.0 | 0.0 |
| Monocytes LPS | 0.0 | 0.0 |
| Macrophages rest | 0.0 | 0.0 |
| Macrophages LPS | 0.0 | 0.0 |
| HUVEC none | 0.0 | 17.7 |
| HUVEC starved | 17.4 | 51.1 |
| HUVEC IL-1beta | 0.0 | 1.7 |
| HUVEC IFN gamma | 3.7 | 11.5 |
| HUVEC TNF alpha + IFN gamma | 0.0 | 3.1 |
| HUVEC TNF alpha + IL4 | 4.3 | 5.1 |
| HUVEC IL-11 | 4.0 | 11.2 |
| Lung Microvascular EC none | 7.2 | 8.1 |
| Lung Microvascular EC TNFalpha + IL-1beta | 0.0 | 0.0 |
| Microvascular Dermal EC none | 8.4 | 14.5 |
| Microsvasular Dermal EC TNFalpha + IL-1beta | 0.0 | 2.2 |
| Bronchial epithelium TNFalpha + IL1beta | 0.0 | 16.3 |
| Small airway epithelium none | 5.9 | 18.8 |
| Small airway epithelium TNFalpha + IL-1beta | 24.3 | 58.6 |
| Coronery artery SMC rest | 0.0 | 2.0 |
| Coronery artery SMC TNFalpha + IL-1beta | 0.0 | 0.0 |
| Astrocytes rest | 3.3 | 13.5 |
| Astrocytes TNFalpha + IL-1beta | 0.0 | 8.6 |
| KU-812 (Basophil) rest | 0.0 | 0.0 |
| KU-812 (Basophil) PMA/ionomycin | 0.0 | 9.7 |
| CCD1106 (Keratinocytes) none | 47.3 | 100.0 |
| CCD1106 (Keratinocytes) TNFalpha + IL-1beta | 9.0 | 53.6 |
| Liver cirrhosis | 32.8 | 9.4 |
| Lupus kidney | 0.0 | 1.6 |
| NCI-H292 none | 3.8 | 3.4 |
| NCI-H292 IL-4 | 8.0 | 19.5 |
| NCI-H292 IL-9 | 0.0 | 4.2 |
| NCI-H292 IL-13 | 13.8 | 7.0 |
| NCI-H292 IFN gamma | 16.2 | 5.7 |
| HPAEC none | 6.7 | 30.1 |
| HPAEC TNF alpha + IL-1beta | 0.0 | 0.0 |
| Lung fibroblast none | 7.6 | 42.0 |
| Lung fibroblast TNFalpha + IL-1beta | 3.1 | 6.3 |
| Lung fibroblast IL-4 | 4.3 | 34.2 |
| Lung fibroblast IL-9 | 12.7 | 27.5 |
| Lung fibroblast IL-13 | 6.8 | 19.9 |
| Lung fibroblast IFN gamma | 30.4 | 51.1 |
| Dermal fibroblast CCD1070 rest | 0.0 | 2.8 |
| Dermal fibroblast CCD1070 TNF alpha | 5.2 | 19.6 |
| Dermal fibroblast CCD1070 IL-1 beta | 0.0 | 2.0 |
| Dermal fibroblast IFN gamma | 28.5 | 32.1 |
| Dermal fibroblast IL-4 | 42.9 | 91.4 |
| IBD Colitis 2 | 2.2 | 5.5 |
| IBD Crohn's | 3.1 | 9.6 |
| Colon | 100.0 | 58.6 |
| Lung | 36.3 | 26.1 |
| Thymus | 0.0 | 0.0 |
| Kidney | 4.0 | 33.0 |

Column A - Rel. Exp. (%) Ag2261, Run 152887762
Column B - Rel. Exp. (%) Ag3035, Run 165242424

TABLE FI
Panel 5 Islet
Column A - Rel. Exp. (%) Ag3035, Run 259234350

| Tissue Name | A |
|---|---|
| 97457_Patient-02go_adipose | 19.3 |
| 97476_Patient-07sk_skeletal muscle | 13.0 |
| 97477_Patient-07ut_uterus | 5.3 |
| 97478_Patient-07pl_placenta | 1.4 |
| 99167_Bayer Patient 1 | 89.5 |
| 97482_Patient-08ut_uterus | 9.6 |
| 97483_Patient-08pl_placenta | 7.4 |
| 97486_Patient-09sk_skeletal muscle | 10.7 |
| 97487_Patient-09ut_uterus | 4.7 |
| 97488_Patient-09pl_placenta | 3.6 |
| 97492_Patient-10ut_uterus | 6.1 |
| 97493_Patient-10pl_placenta | 6.8 |
| 97495_Patient-11go_adipose | 1.6 |
| 97496_Patient-11sk_skeletal muscle | 20.7 |
| 97497_Patient-11ut_uterus | 3.2 |
| 97498_Patient-11pl_placenta | 1.8 |
| 97500_Patient-12go_adipose | 8.5 |
| 97501_Patient-12sk_skeletal muscle | 100.0 |
| 97502_Patient-12ut_uterus | 2.2 |
| 97503_Patient-12pl_placenta | 0.0 |
| 94721_Donor 2 U - A_Mesenchymal | 0.0 |
| Stem Cells | |
| 94722_Donor 2 U - B_Mesenchymal Stem | 1.5 |
| Cells | |
| 94723_Donor 2 U - C_Mesenchymal Stem | 1.5 |
| Cells | |
| 94709_Donor 2 AM - A_adipose | 0.8 |
| 94710_Donor 2 AM - B_adipose | 0.0 |
| 94711_Donor 2 AM - C_adipose | 1.8 |
| 94712_Donor 2 AD - A_adipose | 5.3 |
| 94713_Donor 2 AD - B_adipose | 3.2 |
| 94714_Donor 2 AD - C_adipose | 0.0 |
| 94742_Donor 3 U - A_Mesenchymal Stem | 2.6 |
| Cells | |
| 94743_Donor 3 U - B_Mesenchymal Stem | 2.6 |
| Cells | |
| 94730_Donor 3 AM - A_adipose | 0.8 |
| 94731_Donor 3 AM - B_adipose | 1.7 |
| 94732_Donor 3 AM - C_adipose | 1.5 |
| 94733_Donor 3 AD - A_adipose | 4.8 |
| 94734_Donor 3 AD - B_adipose | 2.0 |
| 94735_Donor 3 AD - C_adipose | 2.5 |
| 77138_Liver_HepG2untreated | 0.0 |
| 73556_Heart_Cardiac stromal cells | 0.0 |
| (primary) | |
| 81735_Small Intestine | 15.6 |
| 72409_Kidney_Proximal Convoluted | 0.0 |
| Tubule | |
| 82685_Small intestine_Duodenum | 5.9 |
| 90650_Adrenal_Adrenocortical adenoma | 2.9 |
| 72410_Kidney_HRCE | 7.5 |
| 72411_Kidney_HRE | 3.3 |
| 73139_Uterus_Uterine smooth muscle cells | 0.0 |

TABLE FJ
general oncology screening panel v 2.4
Column A - Rel. Exp. (%) Ag3035 Run 259737910

| Tissue Name | A |
|---|---|
| Colon cancer 1 | 7.6 |
| Colon cancer NAT 1 | 14.7 |
| Colon cancer 2 | 3.7 |
| Colon cancer NAT 2 | 10.0 |
| Colon cancer 3 | 7.3 |
| Colon cancer NAT 3 | 25.9 |
| Colon malignant cancer 4 | 1.7 |
| Colon normal adjacent tissue 4 | 1.6 |
| Lung cancer 1 | 9.5 |
| Lung NAT 1 | 2.9 |
| Lung cancer 2 | 42.3 |
| Lung NAT 2 | 23.3 |
| Squamous cell carcinoma 3 | 73.7 |
| Lung NAT 3 | 1.3 |
| metastatic melanoma 1 | 37.1 |
| Melanoma 2 | 9.4 |
| Melanoma 3 | 1.4 |
| metastatic melanoma 4 | 100.0 |
| metastatic melanoma 5 | 39.8 |
| Bladder cancer 1 | 2.9 |
| Bladder cancer NAT 1 | 0.0 |
| Bladder cancer 2 | 3.2 |
| Bladder cancer NAT 2 | 0.0 |
| Bladder cancer NAT 3 | 0.0 |
| Bladder cancer NAT 4 | 25.7 |
| Prostate adenocarcinoma 1 | 8.3 |
| Prostate adenocarcinoma 2 | 1.6 |
| Prostate adenocarcinoma 3 | 3.4 |
| Prostate adenocarcinoma 4 | 9.5 |
| Prostate cancer NAT 5 | 2.4 |
| Prostate adenocarcinoma 6 | 0.0 |
| Prostate adenocarcinoma 7 | 4.9 |
| Prostate adenocarcinoma 8 | 0.0 |
| Prostate adenocarcinoma 9 | 20.6 |
| Prostate cancer NAT 10 | 0.4 |
| Kidney cancer 1 | 1.9 |
| KidneyNAT 1 | 8.1 |
| Kidney cancer 2 | 34.4 |
| Kidney NAT 2 | 7.6 |
| Kidney cancer 3 | 12.9 |
| Kidney NAT 3 | 4.5 |
| Kidney cancer 4 | 3.4 |
| Kidney NAT 4 | 5.9 |

AI_comprehensive panel_v1.0 Summary: Ag3035 Highest expression of the CG53054-02 gene was detected in a COPD sample (CT=30). This gene shows widespread expression in this panel. Moderate levels of expression of this gene were detected in samples derived from normal and orthoarthitis/rheumatoid arthritis bone, cartilage, synovium and synovial fluid samples, as well as from normal lung, COPD lung, emphysema, atopic asthma, asthma, allergy, Crohn's disease (normal matched control and diseased), ulcerative colitis (normal matched control and diseased), and psoriasis (normal matched control and diseased). Therapeutic modulation of the activity of this gene or its protein product will ameliorate symptoms/conditions associated with autoimmune and inflammatory disorders, including psoriasis, allergy, asthma, inflammatory bowel disease, rheumatoid arthritis and osteoarthritis.
Oncology_cell_line_screening_panel_v3.2 Summary: Ag3035 Highest expression of this gene was seen in lung cancer cell line DMS-79 (CT=28.6). Moderate to low expression of this gene was also seen in number of cancer cell lines derived from tongue, bone, bladder, pancreatic, cervical, uterine, gastric, colon and lung cancer. Gene or protein expression levels are useful as a marker to detect the presence of these cancers. Therapeutic modulation of the activity of this gene or its protein product is useful in the treatment of these cancers.
Panel 1.3D Summary: Ag2261 This gene was expressed at moderate levels in a number of metabolic tissues, with highest overall expression seen in fetal skeletal muscle (CTs=30.4-31.8). The higher levels of expression in fetal skeletal muscle when compared to adult skeletal muscle suggests that the protein product encoded by this gene may be useful in treating muscular dystrophy, Lesch-Nyhan syndrome, myasthenia gravis and other conditions that result in weak or dystrophic muscle. This gene was also expressed in adipose, thyroid and heart. Since biologic cross-talk between adipose and thyroid is a component of some forms of obesity, therapeutic modulation of the activity of this gene or its protein product is useful for the treatment of metabolic disease, including obesity and Type 2 diabetes.

Ag3035 This probe/primer set recognizes a distinct portion of this gene that shows a distinctive expression pattern when compared to Ag2261. This observation may indicate that the probe/primer sets can distinguish splice variants of this gene. In contrast to the results obtained with Ag2261, expression of this gene was highest in an ovarian cancer cell line (CT=30.6). As was the case for Ag2261, expression of this gene using Ag3035 was also relatively high in fetal skeletal muscle. However, Ag3035 showed higher levels of gene expression in adult skeletal muscle as well as in adult and fetal heart. Most other expression is similar using both probe/primer sets. Please see Ag2261 for additional information.

Panel 2D Summary: Ag2261 This gene was consistently expressed in samples of breast cancer, uterine cancer and lung cancer relative to their respective normal adjacent tissue controls. Gene or protein expression levels are useful as marker to detect breast, uterine and lung cancers. Therapeutic modulation of the activity of this gene or its protein product using nucleic acid, protein, antibody or small molecule drugs is useful in the treatment of breast, lung or uterine cancers.

Panel 4.1D Summary: Ag3035 This probe/primer set recognizes a distinct portion of this gene and shows a distinctive expression pattern relative to probe/primer set Ag2261 in this panel. This observation may indicate that the probe/primer sets can distinguish splice variants of this gene. In contrast to the results obtained with Ag2261 (see panel 4D summary), expression of this gene was highest in normal kidney (CT=30.6). The other expression results for this panel were similar using both probe/primer sets. This gene encodes a WNT-14 homolog and was expressed at moderate to low levels in unstimulated or cytokine-activated keratinocytes as well as in lung and dermal fibroblast preparations (CTs=29-34). Therapeutic modulation of the activity of this gene or its protein product will reduce or eliminate the symptoms of chronic obstructive pulmonary disease, asthma, emphysema, or psoriasis. In addition, due to its known effects on development of vertebrate joints, the protein encoded this gene will reduce or eliminate the symptoms of osetoarthritis (Christine Hartmann and Clifford J. Tabin, 2001, Wnt-14 Plays a Pivotal Role in Inducing Synovial Joint Formation in the Developing Appendicular Skeleton Cell, Vol 104, 341-35).

Panel 4D Summary: Ag2261 This gene was expressed at low levels in colon (CT=33.5). Low but significant levels of expression were also seen in normal lung, keratinocytes and dermal fibroblasts. This gene or the Wnt-14 protein encoded by it may play an important role in the normal homeostasis of these tissues. Therapeutic modulation of the activity of this gene or its protein product is useful to maintain or restore normal function to these organs during inflammation.

Panel 5 Islet Summary: Ag3035 Highest expression of this gene was seen in sample of skeletal muscle from a diabetic patient (CT=31.8). Significant expression of this gene was also seen in pancreatic islet cells. Therapeutic modulation of the activity of this gene or its protein product using nucleic acid, protein, antibody or small molecule drugs is useful in the treatment of metabolic related disease such as obesity and diabetes, especially type II diabetes.

General oncology screening panel_v_{—}2.4 Summary: Ag3035 Highest expression of this gene was detected in a metastatic melanoma sample (CT=31.3). Expression of this gene was also upregulated in prostate, lung and kidney cancers when compared to their appropriate normal adjacent tissue. Gene or protein expression levels are useful for the detection of prostate cancer, lung cancer, kidney cancer and metastatic melanoma. Therapeutic modulation of the activity of this gene or its protein product using nucleic acid, protein, antibody or small molecule drugs is useful for the treatment of these cancers.

G. CG53473-02: Neuromedin B-32 Precursor

Expression of gene CG53473-02 was assessed using the primer-probe set Ag235, described in Table GA. Results of the RTQ-PCR runs are shown in Tables GB and GC.

TABLE GA
Probe Name Ag235

| | | | Start | SEQ ID |
|---|---|---|---|---|
| Primers | Sequences | Length | Position | No |
| Forward | 5′-ttccagcccatcccc | 18 | 225 | 307 |
| | att-3′ | | | |
| Probe | TET-5′-ccccacacctccct | 22 | 253 | 308 |
| | gagggacc-3′-TAMRA | | | |
| Reverse | 5′-cagatcatgactcagctg | 23 | 278 | 309 |
| | cagtc-3′ | | | |

TABLE GB
Panel 1.2

| Tissue Name | A | B |
|---|---|---|
| Endothelial cells | 4.6 | 4.0 |
| Heart (Fetal) | 0.7 | 1.8 |
| Pancreas | 20.6 | 0.6 |
| Pancreatic ca. CAPAN 2 | 4.3 | 2.7 |
| Adrenal Gland | 100.0 | 100.0 |
| Thyroid | 72.2 | 6.2 |
| Salivary gland | 12.6 | 13.8 |
| Pituitary gland | 23.3 | 2.7 |
| Brain (fetal) | 13.7 | 7.7 |
| Brain (whole) | 11.7 | 12.3 |
| Brain (amygdala) | 5.7 | 8.2 |
| Brain (cerebellum) | 4.5 | 11.9 |
| Brain (hippocampus) | 11.3 | 26.2 |
| Brain (thalamus) | 4.2 | 11.9 |
| Cerebral Cortex | 3.9 | 19.5 |
| Spinal cord | 17.4 | 19.5 |
| glio/astro U87-MG | 43.2 | 16.3 |
| glio/astro U-118-MG | 1.0 | 0.3 |
| astrocytoma SW1783 | 1.9 | 0.6 |
| neuro*; met SK-N-AS | 29.3 | 10.7 |
| astrocytoma SF-539 | 1.2 | 0.3 |
| astrocytoma SNB-75 | 0.0 | 0.3 |
| glioma SNB-19 | 25.2 | 11.8 |
| glioma U251 | 5.3 | 2.8 |
| glioma SF-295 | 0.2 | 0.1 |
| Heart | 9.9 | 8.5 |
| Skeletal Muscle | 8.7 | 2.7 |
| Bone marrow | 1.3 | 0.7 |
| Thymus | 0.4 | 0.3 |
| Spleen | 1.8 | 1.1 |
| Lymph node | 1.1 | 3.0 |
| Colorectal Tissue | 0.0 | 0.3 |
| Stomach | 2.6 | 2.4 |
| Small intestine | 1.4 | 1.3 |
| Colon ca. SW480 | 1.0 | 0.1 |
| Colon ca.* SW620 (SW480 met) | 9.2 | 0.8 |
| Colon ca. HT29 | 25.3 | 4.7 |
| Colon ca. HCT-116 | 4.8 | 2.0 |
| Colon ca. CaCo-2 | 8.0 | 3.7 |
| Colon ca. Tissue (ODO3866) | 0.8 | 1.1 |
| Colon ca. HCC-2998 | 42.3 | 14.3 |
| Gastric ca.* (liver met) NCI-N87 | 54.7 | 18.3 |
| Bladder | 11.7 | 3.5 |
| Trachea | 5.6 | 4.0 |
| Kidney | 6.7 | 9.1 |
| Kidney (fetal) | 6.8 | 6.4 |
| Renal ca. 786-0 | 0.3 | 0.2 |
| Renal ca. A498 | 4.3 | 1.5 |
| Renal ca. RXF 393 | 8.7 | 5.3 |
| Renal ca. ACHN | 2.2 | 1.0 |
| Renal ca. UO-31 | 0.6 | 0.3 |
| Renal ca. TK-10 | 1.4 | 0.8 |
| Liver | 0.4 | 1.5 |
| Liver (fetal) | 1.1 | 1.7 |
| Liver ca. (hepatoblast) HepG2 | 1.7 | 1.4 |
| Lung | 3.4 | 3.3 |
| Lung (fetal) | 2.9 | 2.0 |
| Lung ca. (small cell) LX-1 | 7.8 | 1.5 |
| Lung ca. (small cell) NCI-H69 | 0.3 | 0.2 |
| Lung ca. (s. cell var.) SHP-77 | 3.8 | 1.4 |
| Lung ca. (large cell) NCI-H460 | 6.0 | 11.8 |
| Lung ca. (non-sm. cell) A549 | 6.9 | 1.7 |
| Lung ca. (non-s. cell) NCI-H23 | 12.9 | 24.1 |
| Lung ca. (non-s. cell) HOP-62 | 2.4 | 0.5 |
| Lung ca. (non-s. cl) NCI-H522 | 27.0 | 4.0 |
| Lung ca. (squam.) SW 900 | 6.0 | 2.4 |
| Lung ca. (squam.) NCI-H596 | 1.0 | 0.3 |
| Mammary gland | 39.5 | 20.3 |
| Breast ca.* (pl. ef) MCF-7 | 24.0 | 13.0 |
| Breast ca.* (pl. ef) MDA-MB-231 | 0.9 | 0.5 |
| Breast ca.* (pl. ef) T47D | 0.9 | 4.3 |
| Breast ca. BT-549 | 11.5 | 8.5 |
| Breast ca. MDA-N | 13.7 | 8.9 |
| Ovary | 5.8 | 1.8 |
| Ovarian ca. OVCAR-3 | 2.0 | 0.8 |
| Ovarian ca. OVCAR-4 | 1.1 | 0.3 |
| Ovarian ca. OVCAR-5 | 49.0 | 6.5 |
| Ovarian ca. OVCAR-8 | 1.0 | 1.3 |
| Ovarian ca. IGROV-1 | 10.7 | 3.7 |
| Ovarian ca. (ascites) SK-OV-3 | 2.0 | 1.7 |
| Uterus | 2.9 | 2.1 |
| Placenta | 1.8 | 1.1 |
| Prostate | 4.7 | 4.9 |
| Prostate ca.* (bone met) PC-3 | 2.8 | 1.7 |
| Testis | 33.2 | 5.0 |
| Melanoma Hs688(A).T | 1.5 | 0.2 |
| Melanoma* (met) Hs688(B).T | 1.7 | 0.2 |
| Melanoma UACC-62 | 1.5 | 0.4 |
| Melanoma M14 | 5.5 | 2.5 |
| Melanoma LOX IMVI | 6.4 | 1.3 |
| Melanoma* (met) SK-MEL-5 | 6.9 | 2.0 |

Column A - Rel. Exp. (%) Ag235, Run 119215838
Column B - Rel. Exp. (%) Ag235, Run 122741595

TABLE GC
Panel 2D

| Tissue Name | A |
|---|---|
| Normal Colon | 3.3 |
| CC Well to Mod Diff (ODO3866) | 3.1 |
| CC Margin (ODO3866) | 0.8 |
| CC Gr.2 rectosigmoid (ODO3868) | 1.2 |
| CC Margin (ODO3868) | 0.4 |
| CC Mod Diff (ODO3920) | 3.0 |
| CC Margin (ODO3920) | 0.7 |
| CC Gr.2 ascend colon (ODO3921) | 1.4 |
| CC Margin (ODO3921) | 1.1 |
| CC from Partial Hepatectomy (ODO4309) Mets | 5.5 |
| Liver Margin (ODO4309) | 0.7 |
| Colon mets to lung (OD04451-01) | 3.0 |
| Lung Margin (OD04451-02) | 3.0 |
| Normal Prostate 6546-1 | 9.1 |
| Prostate Cancer (OD04410) | 2.7 |
| Prostate Margin (OD04410) | 0.8 |
| Prostate Cancer (OD04720-01) | 1.1 |
| Prostate Margin (OD04720-02) | 2.0 |
| Normal Lung 061010 | 5.1 |
| Lung Met to Muscle (ODO4286) | 7.8 |
| Muscle Margin (ODO4286) | 2.0 |
| Lung Malignant Cancer (OD03126) | 6.3 |
| Lung Margin (OD03126) | 6.7 |
| Lung Cancer (OD04404) | 37.1 |
| Lung Margin (OD04404) | 4.5 |
| Lung Cancer (OD04565) | 19.6 |
| Lung Margin (OD04565) | 2.0 |
| Lung Cancer (OD04237-01) | 8.4 |
| Lung Margin (OD04237-02) | 2.8 |
| Ocular Mel Met to Liver (ODO4310) | 1.6 |
| Liver Margin (ODO4310) | 0.6 |
| Melanoma Mets to Lung (OD04321) | 0.4 |
| Lung Margin (OD04321) | 2.5 |
| Normal Kidney | 1.3 |
| Kidney Ca, Nuclear grade 2 (OD04338) | 5.9 |
| Kidney Margin (OD04338) | 2.7 |
| Kidney Ca Nuclear grade 1/2 (OD04339) | 14.0 |
| Kidney Margin (OD04339) | 5.0 |
| Kidney Ca, Clear cell type (OD04340) | 15.9 |
| Kidney Margin (OD04340) | 3.8 |
| Kidney Ca, Nuclear grade 3 (OD04348) | 4.6 |
| Kidney Margin (OD04348) | 0.9 |
| Kidney Cancer (OD04622-01) | 86.5 |
| Kidney Margin (OD04622-03) | 1.6 |
| Kidney Cancer (OD04450-01) | 4.5 |
| Kidney Margin (OD04450-03) | 1.3 |
| Kidney Cancer 8120607 | 1.5 |
| Kidney Margin 8120608 | 1.7 |
| Kidney Cancer 8120613 | 10.1 |
| Kidney Margin 8120614 | 4.0 |
| Kidney Cancer 9010320 | 100.0 |
| Kidney Margin 9010321 | 7.2 |
| Normal Uterus | 1.3 |
| Uterus Cancer 064011 | 3.1 |
| Normal Thyroid | 66.4 |
| Thyroid Cancer 064010 | 4.5 |
| Thyroid Cancer A302152 | 6.9 |
| Thyroid Margin A302153 | 9.9 |
| Normal Breast | 7.4 |
| Breast Cancer (OD04566) | 0.7 |
| Breast Cancer (OD04590-01) | 5.4 |
| Breast Cancer Mets (OD04590-03) | 11.8 |
| Breast Cancer Metastasis (OD04655-05) | 1.4 |
| Breast Cancer 064006 | 1.5 |
| Breast Cancer 1024 | 8.8 |
| Breast Cancer 9100266 | 1.7 |
| Breast Margin 9100265 | 0.9 |
| Breast Cancer A209073 | 1.1 |
| Breast Margin A209073 | 2.4 |
| Normal Liver | 0.7 |
| Liver Cancer 064003 | 0.7 |
| Liver Cancer 1025 | 0.8 |
| Liver Cancer 1026 | 1.9 |
| Liver Cancer 6004-T | 0.8 |
| Liver Tissue 6004-N | 7.8 |
| Liver Cancer 6005-T | 2.6 |
| Liver Tissue 6005-N | 0.7 |
| Normal Bladder | 4.5 |
| Bladder Cancer 1023 | 2.2 |
| Bladder Cancer A302173 | 4.2 |
| Bladder Cancer (OD04718-01) | 16.5 |
| Bladder Normal Adjacent (OD04718-03) | 2.6 |
| Normal Ovary | 5.0 |
| Ovarian Cancer 064008 | 6.7 |
| Ovarian Cancer (OD04768-07) | 10.2 |
| Ovary Margin (OD04768-08) | 0.1 |
| Normal Stomach | 2.6 |
| Gastric Cancer 9060358 | 0.0 |
| Stomach Margin 9060359 | 0.4 |
| Gastric Cancer 9060395 | 3.2 |
| Stomach Margin 9060394 | 2.2 |
| Gastric Cancer 9060397 | 9.2 |
| Stomach Margin 9060396 | 2.7 |
| Gastric Cancer 064005 | 2.8 |

Column A - Rel. Exp. (%) AG235, Run 145728457

Panel 1.2 Summary: Ag235 Highest expression of the CG53473-02 gene was seen in adrenal gland (CTs=23-26). Significant expression of this gene was also detected in pancreas, adrenal gland, thyroid, pituitary gland, skeletal muscle, heart, liver and the gastrointestinal tract. Therapeutic modulation of the activity of this gene or its protein product is useful in the treatment of endocrine/metabolically related diseases, such as obesity and diabetes.

In addition, this gene was expressed at high to moderate levels in all regions of the central nervous system examined, including amygdala, hippocampus, substantia nigra, thalamus, cerebellum, cerebral cortex, and spinal cord. Therapeutic modulation of the activity of this gene or its protein product is useful in the treatment of central nervous system disorders such as Alzheimer's disease, Parkinson's disease, epilepsy, multiple sclerosis, schizophrenia and depression.

High expression of this gene was also seen in a number of cancer cell lines derived from pancreatic, gastric, colon, lung, liver, renal, breast, ovarian, prostate, squamous cell carcinoma, melanoma and brain cancers. Gene or protein expression levels are useful as a marker for these cancers. Therapeutic modulation of the activity of this gene or its protein product is useful in the treatment of pancreatic, gastric, colon, lung, liver, renal, breast, ovarian, prostate, melanoma and brain cancers.

Panel 2D Summary: Ag235 Highest expression of this gene was detected in a kidney cancer sample (CT=27.9). This gene was overexpressed in a number of kidney, gastric, ovarian, bladder, breast and lung cancers relative to the appropriate normal tissues. Gene or protein expression levels are useful for the detection of these cancers. Therapeutic modulation of the activity of this gene or its protein product using nucleic acid, protein, antibody or small molecule drugs is useful in the treatment of kidney, gastric, ovarian, bladder, breast and lung cancer.

H. CG55184-03: Cerebellin

Expression of gene CG55184-03 was assessed using the primer-probe set Ag1161, described in Table HA. Results of the RTQ-PCR runs are shown in Tables HB, HC, HD and HE.

TABLE HA
Probe Name Ag1161

| | | | Start | SEQ ID |
|---|---|---|---|---|
| Primers | Sequences | Length | Position | No |
| Forward | 5′-aactccaaggtcgcct | 19 | 205 | 310 |
| | tct-3′ | | | |
| Probe | TET-5′-aaccacgagccatc | 23 | 241 | 311 |
| | cgagatgag-3′-TAMRA | | | |
| Reverse | 5′-agtaaatgatgcgcgtct | 21 | 266 | 312 |
| | tgt-3′ | | | |

TABLE HB
CNS neurodegeneration v1.0

| Tissue Name | A | B |
|---|---|---|
| AD 1 Hippo | 5.6 | 5.6 |
| AD 2 Hippo | 55.9 | 49.7 |
| AD 3 Hippo | 20.6 | 15.1 |
| AD 4 Hippo | 100.0 | 100.0 |
| AD 5 Hippo | 31.0 | 40.9 |
| AD 6 Hippo | 8.2 | 8.2 |
| Control 2 Hippo | 29.3 | 30.4 |
| Control 4 Hippo | 10.7 | 10.0 |
| Control (Path) 3 Hippo | 19.8 | 16.0 |
| AD 1 Temporal Ctx | 10.3 | 8.7 |
| AD 2 Temporal Ctx | 25.0 | 25.9 |
| AD 3 Temporal Ctx | 31.2 | 36.3 |
| AD 4 Temporal Ctx | 44.8 | 38.4 |
| AD 5 Inf Temporal Ctx | 25.2 | 25.0 |
| AD 5 Sup Temporal Ctx | 17.8 | 19.5 |
| AD 6 Inf Temporal Ctx | 16.4 | 16.4 |
| AD 6 Sup Temporal Ctx | 25.2 | 26.6 |
| Control 1 Temporal Ctx | 30.6 | 42.6 |
| Control 2 Temporal Ctx | 29.3 | 31.6 |
| Control 3 Temporal Ctx | 69.7 | 58.6 |
| Control 3 Temporal Ctx | 29.1 | 29.1 |
| Control (Path) 1 Temporal Ctx | 41.8 | 40.3 |
| Control (Path) 2 Temporal Ctx | 50.0 | 44.8 |
| Control (Path) 3 Temporal Ctx | 22.5 | 33.2 |
| Control (Path) 4 Temporal Ctx | 43.2 | 44.8 |
| AD 1 Occipital Ctx | 20.7 | 22.2 |
| AD 2 Occipital Ctx (Missing) | 0.9 | 0.8 |
| AD 3 Occipital Ctx | 27.2 | 26.2 |
| AD 4 Occipital Ctx | 59.5 | 70.2 |
| AD 5 Occipital Ctx | 34.4 | 37.9 |
| AD 6 Occipital Ctx | 12.8 | 10.7 |
| Control 1 Occipital Ctx | 19.9 | 15.8 |
| Control 2 Occipital Ctx | 22.5 | 18.9 |
| Control 3 Occipital Ctx | 45.4 | 19.2 |
| Control 4 Occipital Ctx | 20.0 | 24.7 |
| Control (Path) 1 Occipital Ctx | 30.6 | 35.4 |
| Control (Path) 2 Occipital Ctx | 49.3 | 48.6 |
| Control (Path) 3 Occipital Ctx | 6.6 | 8.9 |
| Control (Path) 4 Occipital Ctx | 41.8 | 49.0 |
| Control 1 Parietal Ctx | 29.1 | 0.1 |
| Control 2 Parietal Ctx | 21.9 | 19.5 |
| Control 3 Parietal Ctx | 40.1 | 46.7 |
| Control (Path) 1 Parietal Ctx | 54.7 | 49.7 |
| Control (Path) 2 Parietal Ctx | 46.0 | 47.6 |
| Control (Path) 3 Parietal Ctx | 23.2 | 18.6 |
| Control (Path) 4 Parietal Ctx | 43.2 | 45.7 |

Column A - Rel. Exp. (%) Ag1161, Run 206992277
Column B - Rel. Exp. (%) Ag1161, Run 230512498

TABLE HC
General screening panel v1.7

| Tissue Name | A |
|---|---|
| Adipose | 0.4 |
| HUVEC | 0.0 |
| Melanoma* Hs688(A).T | 0.0 |
| Melanoma* Hs688(B).T | 0.0 |
| Melanoma (met) SK-MEL-5 | 0.0 |
| Testis | 5.6 |
| Prostate ca. (bone met) PC-3 | 0.0 |
| Prostate ca. DU145 | 0.0 |
| Prostate pool | 0.1 |
| Uterus pool | 0.1 |
| Ovarian ca. OVCAR-3 | 0.0 |
| Ovarian ca. (ascites) SK-OV-3 | 0.0 |
| Ovarian ca. OVCAR-4 | 0.0 |
| Ovarian ca. OVCAR-5 | 0.0 |
| Ovarian ca. IGROV-1 | 0.0 |
| Ovarian ca. OVCAR-8 | 0.0 |
| Ovary | 12.6 |
| Breast ca. MCF-7 | 0.1 |
| Breast ca. MDA-MB-231 | 0.0 |
| Breast ca. BT-549 | 0.0 |
| Breast ca. T47D | 0.0 |
| Breast pool | 0.8 |
| Trachea | 0.6 |
| Lung | 0.2 |
| Fetal Lung | 0.7 |
| Lung ca. NCI-N417 | 0.0 |
| Lung ca. LX-1 | 0.0 |
| Lung ca. NCI-H146 | 0.3 |
| Lung ca. SHP-77 | 0.0 |
| Lung ca. NCI-H23 | 0.0 |
| Lung ca. NCI-H460 | 0.0 |
| Lung ca. HOP-62 | 0.0 |
| Lung ca. NCI-H522 | 0.0 |
| Lung ca. DMS-114 | 0.0 |
| Liver | 0.8 |
| Fetal Liver | 0.4 |
| Kidney pool | 2.7 |
| Fetal Kidney | 3.4 |
| Renal ca. 786-0 | 0.0 |
| Renal ca. A498 | 0.0 |
| Renal ca. ACHN | 0.0 |
| Renal ca. UO-31 | 0.0 |
| Renal ca. TK-10 | 0.0 |
| Bladder | 0.4 |
| Gastric ca. (liver met.) NCI-N87 | 0.0 |
| Stomach | 0.0 |
| Colon ca. SW-948 | 0.1 |
| Colon ca. SW480 | 0.0 |
| Colon ca. (SW480 met) SW620 | 0.0 |
| Colon ca. HT29 | 0.0 |
| Colon ca. HCT-116 | 0.0 |
| Colon cancer tissue | 0.1 |
| Colon ca. SW1116 | 0.0 |
| Colon ca. Colo-205 | 0.0 |
| Colon ca. SW-48 | 0.0 |
| Colon | 0.0 |
| Small Intestine | 0.3 |
| Fetal Heart | 0.0 |
| Heart | 0.2 |
| Lymph Node pool 1 | 0.8 |
| Lymph Node pool 2 | 0.1 |
| Fetal Skeletal Muscle | 0.2 |
| Skeletal Muscle pool | 0.6 |
| Skeletal Muscle | 1.8 |
| Spleen | 3.2 |
| Thymus | 1.1 |
| CNS cancer (glio/astro) SF-268 | 0.0 |
| CNS cancer (glio/astro) T98G | 0.0 |
| CNS cancer (neuro; met) SK-N-AS | 0.1 |
| CNS cancer (astro) SF-539 | 0.0 |
| CNS cancer (astro) SNB-75 | 0.0 |
| CNS cancer (glio) SNB-19 | 0.0 |
| CNS cancer (glio) SF-295 | 0.0 |
| Brain (Amygdala) | 60.3 |
| Brain (Cerebellum) | 0.8 |
| Brain (Fetal) | 20.6 |
| Brain (Hippocampus) | 21.3 |
| Cerebral Cortex pool | 36.9 |
| Brain (Substantia nigra) | 26.6 |
| Brain (Thalamus) | 42.6 |
| Brain (Whole) | 100.0 |
| Spinal Cord | 1.9 |
| Adrenal Gland | 36.6 |
| Pituitary Gland | 0.0 |
| Salivary Gland | 0.0 |
| Thyroid | 1.0 |
| Pancreatic ca. PANC-1 | 0.0 |
| Pancreas pool | 0.8 |

Column A - Rel. Exp. (%) Ag1161, Run 317667428

TABLE HD
Panel 2.2

| Tissue Name | A |
|---|---|
| Normal Colon | 4.8 |
| Colon cancer (OD06064) | 0.0 |
| Colon Margin (OD06064) | 0.0 |
| Colon cancer (OD06159) | 0.0 |
| Colon Margin (OD06159) | 0.0 |
| Colon cancer (OD06297-04) | 0.0 |
| Colon Margin (OD06297-05) | 0.0 |
| CC Gr.2 ascend colon (ODO3921) | 0.0 |
| CC Margin (ODO3921) | 4.7 |
| Colon cancer metastasis (OD06104) | 0.0 |
| Lung Margin (OD06104) | 3.6 |
| Colon mets to lung (OD04451-01) | 0.0 |
| Lung Margin (OD04451-02) | 4.0 |
| Normal Prostate | 4.5 |
| Prostate Cancer (OD04410) | 0.0 |
| Prostate Margin (OD04410) | 0.0 |
| Normal Ovary | 26.4 |
| Ovarian cancer (OD06283-03) | 0.0 |
| Ovarian Margin (OD06283-07) | 100.0 |
| Ovarian Cancer 064008 | 47.6 |
| Ovarian cancer (OD06145) | 35.4 |
| Ovarian Margin (OD06145) | 33.2 |
| Ovarian cancer (OD06455-03) | 0.0 |
| Ovarian Margin (OD06455-07) | 22.2 |
| Normal Lung | 5.3 |
| Invasive poor diff. lung adeno (ODO4945-01 | 0.0 |
| Lung Margin (ODO4945-03) | 0.0 |
| Lung Malignant Cancer (OD03126) | 0.0 |
| Lung Margin (OD03126) | 4.4 |
| Lung Cancer (OD05014A) | 0.0 |
| Lung Margin (OD05014B) | 10.2 |
| Lung cancer (OD06081) | 0.0 |
| Lung Margin (OD06081) | 0.0 |
| Lung Cancer (OD04237-01) | 0.0 |
| Lung Margin (OD04237-02) | 45.4 |
| Ocular Melanoma Metastasis | 0.0 |
| Ocular Melanoma Margin (Liver) | 6.1 |
| Melanoma Metastasis | 0.0 |
| Melanoma Margin (Lung) | 4.7 |
| Normal Kidney | 0.0 |
| Kidney Ca, Nuclear grade 2 (OD04338) | 8.4 |
| Kidney Margin (OD04338) | 0.0 |
| Kidney Ca Nuclear grade 1/2 (OD04339) | 2.2 |
| Kidney Margin (OD04339) | 27.7 |
| Kidney Ca, Clear cell type (OD04340) | 33.9 |
| Kidney Margin (OD04340) | 8.8 |
| Kidney Ca, Nuclear grade 3 (OD04348) | 9.7 |
| Kidney Margin (OD04348) | 14.0 |
| Kidney malignant cancer (OD06204B) | 5.2 |
| Kidney normal adjacent tissue (OD06204E) | 8.8 |
| Kidney Cancer (OD04450-01) | 0.0 |
| Kidney Margin (OD04450-03) | 4.9 |
| Kidney Cancer 8120613 | 0.0 |
| Kidney Margin 8120614 | 5.2 |
| Kidney Cancer 9010320 | 21.5 |
| Kidney Margin 9010321 | 0.0 |
| Kidney Cancer 8120607 | 5.2 |
| Kidney Margin 8120608 | 0.0 |
| Normal Uterus | 3.6 |
| Uterine Cancer 064011 | 0.0 |
| Normal Thyroid | 0.0 |
| Thyroid Cancer 064010 | 40.9 |
| Thyroid Cancer A302152 | 9.0 |
| Thyroid Margin A302153 | 3.7 |
| Normal Breast | 0.0 |
| Breast Cancer (OD04566) | 5.2 |
| Breast Cancer 1024 | 0.0 |
| Breast Cancer (OD04590-01) | 8.6 |
| Breast Cancer Mets (OD04590-03) | 10.5 |
| Breast Cancer Metastasis (OD04655-05) | 0.0 |
| Breast Cancer 064006 | 5.3 |
| Breast Cancer 9100266 | 0.0 |
| Breast Margin 9100265 | 8.1 |
| Breast Cancer A209073 | 9.7 |
| Breast Margin A2090734 | 10.4 |
| Breast cancer (OD06083) | 4.7 |
| Breast cancer node metastasis (OD06083) | 7.7 |
| Normal Liver | 17.6 |
| Liver Cancer 1026 | 0.0 |
| Liver Cancer 1025 | 17.9 |
| Liver Cancer 6004-T | 0.0 |
| Liver Tissue 6004-N | 15.7 |
| Liver Cancer 6005-T | 0.0 |
| Liver Tissue 6005-N | 0.0 |
| Liver Cancer 064003 | 0.0 |
| Normal Bladder | 0.0 |
| Bladder Cancer 1023 | 0.0 |
| Bladder Cancer A302173 | 0.0 |
| Normal Stomach | 0.0 |
| Gastric Cancer 9060397 | 0.0 |
| Stomach Margin 9060396 | 8.7 |
| Gastric Cancer 9060395 | 16.7 |
| Stomach Margin 9060394 | 0.0 |
| Gastric Cancer 064005 | 0.0 |

Column A - Rel. Exp. (%) Ag1161, Run 173769890

TABLE HE
Panel 4D

| Tissue Name | A |
|---|---|
| Secondary Th1 act | 0.0 |
| Secondary Th2 act | 0.0 |
| Secondary Tr1 act | 0.0 |
| Secondary Th1 rest | 0.0 |
| Secondary Th2 rest | 0.0 |
| Secondary Tr1 rest | 0.0 |
| Primary Th1 act | 0.0 |
| Primary Th2 act | 0.0 |
| Primary Tr1 act | 0.0 |
| Primary Th1 rest | 0.0 |
| Primary Th2 rest | 0.0 |
| Primary Tr1 rest | 0.0 |
| CD45RA CD4 lymphocyte act | 0.0 |
| CD45RO CD4 lymphocyte act | 0.0 |
| CD8 lymphocyte act | 0.0 |
| Secondary CD8 lymphocyte rest | 0.0 |
| Secondary CD8 lymphocyte act | 0.0 |
| CD4 lymphocyte none | 0.0 |
| 2ry Th1/Th2/Tr1_anti-CD95 CH11 | 0.0 |
| LAK cells rest | 0.0 |
| LAK cells IL-2 | 0.0 |
| LAK cells IL-2 + IL-12 | 0.0 |
| LAK cells IL-2 + IFN gamma | 0.0 |
| LAK cells IL-2 + IL-18 | 0.0 |
| LAK cells PMA/ionomycin | 0.0 |
| NK Cells IL-2 rest | 0.0 |
| Two Way MLR 3 day | 0.0 |
| Two Way MLR 5 day | 0.0 |
| Two Way MLR 7 day | 0.0 |
| PBMC rest | 0.0 |
| PBMC PWM | 0.0 |
| PBMC PHA-L | 0.0 |
| Ramos (B cell) none | 0.0 |
| Ramos (B cell) ionomycin | 0.0 |
| B lymphocytes PWM | 0.0 |
| B lymphocytes CD40L and IL-4 | 0.0 |
| EOL-1 dbcAMP | 0.0 |
| EOL-1 dbcAMP PMA/ionomycin | 0.0 |
| Dendritic cells none | 0.0 |
| Dendritic cells LPS | 0.0 |
| Dendritic cells anti-CD40 | 0.0 |
| Monocytes rest | 0.0 |
| Monocytes LPS | 0.0 |
| Macrophages rest | 0.0 |
| Macrophages LPS | 18.2 |
| HUVEC none | 0.0 |
| HUVEC starved | 0.0 |
| HUVEC IL-1beta | 0.0 |
| HUVEC IFN gamma | 0.0 |
| HUVEC TNF alpha + IFN gamma | 0.0 |
| HUVEC TNF alpha + IL4 | 0.0 |
| HUVEC IL-11 | 0.0 |
| Lung Microvascular EC none | 0.0 |
| Lung Microvascular EC TNFalpha + IL-1beta | 0.0 |
| Microvascular Dermal EC none | 0.0 |
| Microsvasular Dermal EC TNFalpha + IL-1beta | 0.0 |
| Bronchial epithelium TNFalpha + IL1beta | 0.0 |
| Small airway epithelium none | 0.0 |
| Small airway epithelium TNFalpha + IL-1beta | 0.0 |
| Coronery artery SMC rest | 0.0 |
| Coronery artery SMC TNFalpha + IL-1beta | 0.0 |
| Astrocytes rest | 0.0 |
| Astrocytes TNFalpha + IL-1beta | 0.0 |
| KU-812 (Basophil) rest | 0.0 |
| KU-812 (Basophil) PMA/ionomycin | 0.0 |
| CCD1106 (Keratinocytes) none | 0.0 |
| CCD1106 (Keratinocytes) TNFalpha + IL-1beta | 0.0 |
| Liver cirrhosis | 68.3 |
| Lupus kidney | 26.6 |
| NCI-H292 none | 0.0 |
| NCI-H292 IL-4 | 0.0 |
| NCI-H292 IL-9 | 0.0 |
| NCI-H292 IL-13 | 0.0 |
| NCI-H292 IFN gamma | 0.0 |
| HPAEC none | 0.0 |
| HPAEC TNF alpha + IL-1 beta | 0.0 |
| Lung fibroblast none | 0.0 |
| Lung fibroblast TNF alpha + IL-1 beta | 0.0 |
| Lung fibroblast IL-4 | 0.0 |
| Lung fibroblast IL-9 | 0.0 |
| Lung fibroblast IL-13 | 0.0 |
| Lung fibroblast IFN gamma | 0.0 |
| Dermal fibroblast CCD1070 rest | 0.0 |
| Dermal fibroblast CCD1070 TNF alpha | 0.0 |
| Dermal fibroblast CCD1070 IL-1 beta | 0.0 |
| Dermal fibroblast IFN gamma | 0.0 |
| Dermal fibroblast IL-4 | 0.0 |
| IBD Colitis 2 | 0.0 |
| IBD Crohn's | 8.8 |
| Colon | 81.8 |
| Lung | 100.0 |
| Thymus | 26.2 |
| Kidney | 25.9 |

Column A - Rel. Exp. (%) Ag1161, Run 139841942

CNS_neurodegeneration_v1.0 Summary: Ag1161 Expression of the CG55184-03 gene was down-regulated in the temporal cortex of Alzheimer's disease patients when compared with normal patients. Therefore, up-regulation of this gene or its protein product or treatment with specific agonists for this receptor is useful in reversing the dementia/memory loss and neuronal death associated with this disease.
General_screening_panel_v1.7 Summary: Ag1161 Highest expression of this gene was detected in whole brain (CT=26). This gene showed brain preferential expression with high expression seen in all the regions of the central nervous system examined, including amygdala, hippocampus, substantia nigra, thalamus, cerebellum, cerebral cortex, and spinal cord. Gene or protein expression levels are useful as a marker for brain. Therapeutic modulation of the activity of this gene or its protein product is useful in the treatment of central nervous system disorders such as Alzheimer's disease, Parkinson's disease, epilepsy, multiple sclerosis, schizophrenia and depression.

Moderate to low expression of this gene was also seen in tissues with metabolic/endocrine function including pancreas, adipose, adrenal gland, thyroid, skeletal muscle, liver and small intestine. Therapeutic modulation of the activity of this gene or its protein product is useful in the treatment of endocrine/metabolically related diseases, such as obesity and diabetes.

Panel 2.2 Summary: Ag1161 Highest expression of this gene was detected in normal ovarian tissue (CT=32). Expression of this gene was upregulated in normal ovarian and lung samples relative to corresponding cancer samples. Therapeutic modulation of the activity of this gene or its protein product using nucleic acid, protein, antibody or small molecule drugs is useful in the treatment of ovarian and lung cancers.

Low expression of this gene was also detected in a thyroid cancer sample. Therapeutic modulation of the activity of this gene or its protein product using nucleic acid, protein, antibody or small molecule drugs is useful in the treatment of thyroid cancer.

Panel 4D Summary: Ag1161 This gene was expressed at low levels in normal lung and colon (CTs=34). Expression of this gene was downregulated in the colon from a Crohn's disease patient was reduced. Therapeutic modulation of the activity of this gene or its protein product is useful in the treatment of inflammatory bowel diseases, such as Crohn's. Low expression of this gene was also seen in liver cirrhosis sample. Therapeutic modulation of the activity of this gene or its protein product is useful in the treatment of liver cirrhosis.

I. CG55274-05: Diazepam-binding Inhibitor

Expression of gene CG55274-05 was assessed using the primer-probe set Ag497, described in Table IA. Results of the RTQ-PCR runs are shown in Tables IB and IC.

TABLE IA
Probe Name Ag497

| | | | Start | SEQ ID |
|---|---|---|---|---|
| Primers | Sequences | Length | Position | No |
| Forward | 5′-gcagcatggaccctc | 18 | 181 | 313 |
| | caa-3′ | | | |
| Probe | TET-5′-acactcgttgcatc | 30 | 201 | 314 |
| | ttcctttgacaacc | | | |
| | tt-3′-TAMRA | | | |
| Reverse | 5′-tcggctcttttgccttag | 23 | 234 | 315 |
| | aaata-3′ | | | |

TABLE IB
Panel 1.1

| Tissue Name | A |
|---|---|
| Adrenal gland | 0.0 |
| Bladder | 0.0 |
| Brain (amygdala) | 0.0 |
| Brain (cerebellum) | 0.0 |
| Brain (hippocampus) | 0.0 |
| Brain (substantia nigra) | 0.0 |
| Brain (thalamus) | 0.0 |
| Cerebral Cortex | 0.0 |
| Brain (fetal) | 0.0 |
| Brain (whole) | 0.0 |
| glio/astro U-118-MG | 0.0 |
| astrocytoma SF-539 | 0.0 |
| astrocytoma SNB-75 | 0.0 |
| astrocytoma SW1783 | 0.0 |
| glioma U251 | 0.0 |
| glioma SF-295 | 0.0 |
| glioma SNB-19 | 0.0 |
| glio/astro U87-MG | 0.0 |
| neuro*; met SK-N-AS | 0.0 |
| Mammary gland | 0.0 |
| Breast ca. BT-549 | 0.0 |
| Breast ca. MDA-N | 0.0 |
| Breast ca.* (pl. ef) T47D | 0.0 |
| Breast ca.* (pl. ef) MCF-7 | 0.0 |
| Breast ca.* (pl. ef) MDA-MB-231 | 0.0 |
| Small intestine | 0.0 |
| Colorectal | 0.0 |
| Colon ca. HT29 | 0.0 |
| Colon ca. CaCo-2 | 0.0 |
| Colon ca. HCT-15 | 0.0 |
| Colon ca. HCT-116 | 0.0 |
| Colon ca. HCC-2998 | 0.0 |
| Colon ca. SW480 | 0.0 |
| Colon ca.* SW620 (SW480 met) | 0.0 |
| Stomach | 0.0 |
| Gastric ca. (liver met) NCI-N87 | 0.0 |
| Heart | 0.0 |
| Skeletal muscle (Fetal) | 0.0 |
| Skeletal muscle | 0.0 |
| Endothelial cells | 0.0 |
| Heart (Fetal) | 0.0 |
| Kidney | 0.0 |
| Kidney (fetal) | 0.0 |
| Renal ca. 786-0 | 0.0 |
| Renal ca. A498 | 0.0 |
| Renal ca. ACHN | 0.0 |
| Renal ca. TK-10 | 0.0 |
| Renal ca. UO-31 | 0.0 |
| Renal ca. RXF 393 | 0.0 |
| Liver | 0.0 |
| Liver (fetal) | 0.0 |
| Liver ca. (hepatoblast) HepG2 | 0.0 |
| Lung | 0.0 |
| Lung (fetal) | 0.0 |
| Lung ca. (non-s. cell) HOP-62 | 0.0 |
| Lung ca. (large cell)NCI-H460 | 0.0 |
| Lung ca. (non-s. cell) NCI-H23 | 0.0 |
| Lung ca. (non-s. cl) NCI-H522 | 0.0 |
| Lung ca. (non-sm. cell) A549 | 0.0 |
| Lung ca. (s. cell var.) SHP-77 | 0.0 |
| Lung ca. (small cell) LX-1 | 0.0 |
| Lung ca. (small cell) NCI-H69 | 12.0 |
| Lung ca. (squam.) SW 900 | 0.0 |
| Lung ca. (squam.) NCI-H596 | 0.0 |
| Lymph node | 0.0 |
| Spleen | 0.0 |
| Thymus | 0.0 |
| Ovary | 0.0 |
| Ovarian ca. IGROV-1 | 0.0 |
| Ovarian ca. OVCAR-3 | 0.0 |
| Ovarian ca. OVCAR-4 | 0.0 |
| Ovarian ca. OVCAR-5 | 100.0 |
| Ovarian ca. OVCAR-8 | 0.0 |
| Ovarian ca.* (ascites) SK-OV-3 | 0.0 |
| Pancreas | 0.0 |
| Pancreatic ca. CAPAN 2 | 0.0 |
| Pituitary gland | 0.0 |
| Placenta | 0.0 |
| Prostate | 0.0 |
| Prostate ca.* (bone met) PC-3 | 0.0 |
| Salivary gland | 0.0 |
| Trachea | 0.0 |
| Spinal cord | 0.0 |
| Testis | 0.0 |
| Thyroid | 0.0 |
| Uterus | 0.0 |
| Melanoma M14 | 0.0 |
| Melanoma LOX IMVI | 0.0 |
| Melanoma UACC-62 | 0.0 |
| Melanoma SK-MEL-28 | 0.0 |
| Melanoma* (met) SK-MEL-5 | 0.0 |
| Melanoma Hs688(A).T | 0.0 |
| Melanoma* (met) Hs688(B).T | 0.0 |

Column A - Rel. Exp. (%) Ag497, Run 121136178

TABLE IC
Panel 5 Islet

| Tissue Name | A |
|---|---|
| 97457_Patient-02go_adipose | 0.0 |
| 97476_Patient-07sk_skeletal muscle | 0.0 |
| 97477_Patient-07ut_uterus | 0.0 |
| 97478_Patient-07pl_placenta | 0.0 |
| 99167_Bayer Patient 1 | 0.0 |
| 97482_Patient-08ut_uterus | 0.0 |
| 97483_Patient-08pl_placenta | 0.0 |
| 97486_Patient-09sk_skeletal muscle | 0.0 |
| 97487_Patient-09ut_uterus | 0.0 |
| 97488_Patient-09pl_placenta | 0.0 |
| 97492_Patient-10ut_uterus | 0.0 |
| 97493_Patient-10pl_placenta | 12.5 |
| 97495_Patient-11go_adipose | 0.0 |
| 97496_Patient-11sk_skeletal muscle | 6.3 |
| 97497_Patient-11ut_uterus | 0.0 |
| 97498_Patient-11pl_placenta | 0.0 |
| 97500_Patient-12go_adipose | 0.0 |
| 97501_Patient-12sk_skeletal muscle | 100.0 |
| 97502_Patient-12ut_uterus | 8.1 |
| 97503_Patient-12pl_placenta | 0.0 |
| 94721_Donor 2 U - A_Mesenchymal Stem Cells | 1.3 |
| 94722_Donor 2 U - B_Mesenchymal Stem Cells | 0.0 |
| 94723_Donor 2 U - C_Mesenchymal Stem Cells | 0.0 |
| 94709_Donor 2 AM - A_adipose | 0.0 |
| 94710_Donor 2 AM - B_adipose | 0.0 |
| 94711_Donor 2 AM - C_adipose | 0.0 |
| 94712_Donor 2 AD - A_adipose | 0.0 |
| 94713_Donor 2 AD - B_adipose | 0.0 |
| 94714_Donor 2 AD - C_adipose | 0.0 |
| 94742_Donor 3 U - A_Mesenchymal Stem Cells | 0.0 |
| 94743_Donor 3 U - B_Mesenchymal Stem Cells | 0.0 |
| 94730_Donor 3 AM - A_adipose | 10.5 |
| 94731_Donor 3 AM - B_adipose | 0.0 |
| 94732_Donor 3 AM - C_adipose | 0.0 |
| 94733_Donor 3 AD - A_adipose | 0.0 |
| 94734_Donor 3 AD - B_adipose | 24.1 |
| 94735_Donor 3 AD - C_adipose | 0.0 |
| 77138_Liver_HepG2untreated | 0.0 |
| 73556_Heart_Cardiac stromal cells (primary) | 0.0 |
| 81735_Small Intestine | 20.7 |
| 72409_Kidney_Proximal Convoluted Tubule | 0.0 |
| 82685_Small intestine_Duodenum | 0.0 |
| 90650_Adrenal_Adrenocortical adenoma | 0.0 |
| 72410_Kidney_HRCE | 0.0 |
| 72411_Kidney_HRE | 0.0 |
| 73139_Uterus_Uterine smooth muscle cells | 0.0 |

Column A - Rel. Exp. (%) Ag497, Run 323591176

Panel 1.1 Summary: Ag497 Low expression of the CG55274-05 gene was restricted to the ovarian cancer cell line OVCAR-5 (CT=33.6). Gene or protein expression levels are useful as a marker to detect the presence of ovarian cancer. Therapeutic modulation of the activity of this gene or its protein product is useful in the treatment of ovarian cancer.
Panel 5 Islet Summary: Ag497 Low expression of this gene was mainly detected in a skeletal muscle sample from a diabetic patient on insulin (CT=33.6). The CG55274-05 gene encodes Diazepam-binding inhibitor, a member of the endozepine/acetyl CoA binding protein (ACBP)/diazepam binding inhibitor (DBI) family. ACBP is known to affect intracellular calcium levels via release from the sarcoplasmic reticulum in muscle, via the ryanodine receptor, and possibly the mitochondria (Fulceri R, Knudsen J, Giunti R, Volpe P, Nori A, Benedetti A. Fatty acyl-CoA-acyl-CoA-binding protein complexes activate the Ca2+ release channel of skeletal muscle sarcoplasmic reticulum. Biochem J Jul. 15, 1997;325 (Pt 2):423-8; Fulceri R, Giunti R, Knudsen J, Leuzzi R, Kardon T, Benedetti A. Rapamycin inhibits activation of ryanodine receptors from skeletal muscle by the fatty acyl CoA-acyl CoA binding protein complex. Biochem Biophys Res Commun Oct. 22, 1999;264(2):409-12). Since the activity of many metabolic enzymes is regulated by intracellular calcium, ACBP could play an important role in many aspects of energy metabolism. Furthermore, the peptides produced from ACBP act as hormones or paracrine factors to influence metabolism globally. One ACBP-derived peptide (octadecaneuropeptide: ODN-ACBP33-50) exerts this action through several mechanisms. One mechanism influences nutrient absorption through the stimulation of CCK secretion and the subsequent secretion by the exocrine pancreas (Herzig K H; Schon I; Tatemoto K; Ohe Y; Li Y; Folsch U R; Owyang C. Diazepam binding inhibitor is a potent cholecystokinin-releasing peptide in the intestine. Proc. Nat. Acad. Sci. 1996; 93: 7927-7932). At the same time ODN inhibits glucose-stimulated insulin secretion from the endocrine pancreas [10]. In addition, intracerebroventricular administration of ODN has anorexigenic effects in rats (de Mateos-Verchere J G, Leprince J, Tonon M C, Vaudry H, Costentin J. The octadecaneuropeptide [diazepam-binding inhibitor (33-50)] exerts potent anorexigenic effects in rodents. Eur J Pharmacol Mar. 2, 2001;414(2-3):225-31). Full-length ACBP and peptides derived from the parent polypeptide participate in several different feedback loops influencing metabolism at many levels. Based upon the specific expression of this gene in skeletal muscle from diabetic patient and that ODN has broad-ranging effects on physiologic processes, ODN-related peptides from the CG55274-05 gene, encoding an ACBP-like protein, are potential protein therapeutics for the treatment of metabolic disorders such as obesity and diabetes.

J. CG55379-01 and CG55379-04: hNOPE

Expression of gene CG55379-01 and CG55379-04 was assessed using the primer-probe set Ag902, described in Table JA. Results of the RTQ-PCR runs are shown in Tables JB, JC, JD, JE, JF, JG and JH.

TABLE JA
Probe Name Ag902

| | | | Start | SEQ ID |
|---|---|---|---|---|
| Primers | Sequences | Length | Position | No |
| Forward | 5′-atcaaacagctccacatc | 21 | 2289 | 316 |
| | cat-3′ | | | |
| Probe | TET-5′-aaaagccagatttc | 26 | 2324 | 317 |
| | accacagtcaag-3′-TAMRA | | | |
| Reverse | 5′-agcgcacagtgtagttga | 22 | 2350 | 318 |
| | caat-3′ | | | |

TABLE JB
CNS neurodegeneration v1.0

| Tissue Name | A |
|---|---|
| AD 1 Hippo | 53.2 |
| AD 2 Hippo | 62.4 |
| AD 3 Hippo | 27.0 |
| AD 4 Hippo | 38.2 |
| AD 5 Hippo | 19.8 |
| AD 6 Hippo | 63.3 |
| Control 2 Hippo | 31.9 |
| Control 4 Hippo | 100.0 |
| Control (Path) 3 Hippo | 28.5 |
| AD 1 Temporal Ctx | 50.3 |
| AD 2 Temporal Ctx | 56.6 |
| AD 3 Temporal Ctx | 41.2 |
| AD 4 Temporal Ctx | 35.4 |
| AD 5 Inf Temporal Ctx | 27.2 |
| AD 5 Sup Temporal Ctx | 48.0 |
| AD 6 Inf Temporal Ctx | 38.7 |
| AD 6 Sup Temporal Ctx | 46.0 |
| Control 1 Temporal Ctx | 17.4 |
| Control 2 Temporal Ctx | 25.0 |
| Control 3 Temporal Ctx | 25.0 |
| Control 3 Temporal Ctx | 53.6 |
| Control (Path) 1 Temporal Ctx | 23.5 |
| Control (Path) 2 Temporal Ctx | 27.7 |
| Control (Path) 3 Temporal Ctx | 34.9 |
| Control (Path) 4 Temporal Ctx | 27.2 |
| AD 1 Occipital Ctx | 24.8 |
| AD 2 Occipital Ctx (Missing) | 4.6 |
| AD 3 Occipital Ctx | 26.8 |
| AD 4 Occipital Ctx | 29.3 |
| AD 5 Occipital Ctx | 22.4 |
| AD 6 Occipital Ctx | 20.9 |
| Control 1 Occipital Ctx | 29.1 |
| Control 2 Occipital Ctx | 24.3 |
| Control 3 Occipital Ctx | 24.8 |
| Control 4 Occipital Ctx | 56.6 |
| Control (Path) 1 Occipital Ctx | 31.2 |
| Control (Path) 2 Occipital Ctx | 17.2 |
| Control (Path) 3 Occipital Ctx | 26.8 |
| Control (Path) 4 Occipital Ctx | 19.5 |
| Control 1 Parietal Ctx | 30.1 |
| Control 2 Parietal Ctx | 41.2 |
| Control 3 Parietal Ctx | 28.7 |
| Control (Path) 1 Parietal Ctx | 25.5 |
| Control (Path) 2 Parietal Ctx | 34.6 |
| Control (Path) 3 Parietal Ctx | 45.4 |
| Control (Path) 4 Parietal Ctx | 29.9 |

Column A - Rel. Exp. (%) Ag902, Run 207567448

TABLE JC
General screening panel v1.4

| Tissue Name | A |
|---|---|
| Adipose | 2.6 |
| Melanoma* Hs688(A).T | 9.1 |
| Melanoma* Hs688(B).T | 8.7 |
| Melanoma* M14 | 0.2 |
| Melanoma* LOXIMVI | 0.0 |
| Melanoma* SK-MEL-5 | 0.5 |
| Squamous cell carcinoma SCC-4 | 0.2 |
| Testis Pool | 14.2 |
| Prostate ca.* (bone met) PC-3 | 3.0 |
| Prostate Pool | 1.6 |
| Placenta | 63.3 |
| Uterus Pool | 2.4 |
| Ovarian ca. OVCAR-3 | 0.4 |
| Ovarian ca: SK-OV-3 | 4.5 |
| Ovarian ca. OVCAR-4 | 3.4 |
| Ovarian ca. OVCAR-5 | 6.9 |
| Ovarian ca. IGROV-1 | 1.4 |
| Ovarian ca. OVCAR-8 | 14.7 |
| Ovary | 26.6 |
| Breast ca. MCF-7 | 0.4 |
| Breast ca. MDA-MB-231 | 9.9 |
| Breast ca. BT 549 | 1.2 |
| Breast ca. T47D | 12.2 |
| Breast ca. MDA-N | 0.0 |
| Breast Pool | 12.9 |
| Trachea | 3.6 |
| Lung | 10.6 |
| Fetal Lung | 75.8 |
| Lung ca. NCI-N417 | 0.9 |
| Lung ca. LX-1 | 3.8 |
| Lung ca. NCI-H146 | 0.3 |
| Lung ca. SHP-77 | 1.6 |
| Lung ca. A549 | 5.5 |
| Lung ca. NCI-H526 | 1.1 |
| Lung ca. NCI-H23 | 7.3 |
| Lung ca. NCI-H460 | 0.4 |
| Lung ca. HOP-62 | 3.1 |
| Lung ca. NCI-H522 | 47.6 |
| Liver | 0.0 |
| Fetal Liver | 29.1 |
| Liver ca. HepG2 | 0.2 |
| Kidney Pool | 11.0 |
| Fetal Kidney | 22.8 |
| Renal ca. 786-0 | 0.7 |
| Renal ca. A498 | 0.6 |
| Renal ca. ACHN | 7.2 |
| Renal ca. UO-31 | 45.7 |
| Renal ca. TK-10 | 32.8 |
| Bladder | 3.9 |
| Gastric ca. (liver met.) NCI-N87 | 0.2 |
| Gastric ca. KATO III | 0.1 |
| Colon ca. SW-948 | 0.2 |
| Colon ca. SW480 | 6.4 |
| Colon ca.* (SW480 met) SW620 | 4.0 |
| Colon ca. HT29 | 0.0 |
| Colon ca. HCT-116 | 0.4 |
| Colon ca. CaCo-2 | 7.0 |
| Colon cancer tissue | 4.4 |
| Colon ca. SW1116 | 0.0 |
| Colon ca. Colo-205 | 0.1 |
| Colon ca. SW-48 | 0.0 |
| Colon Pool | 6.8 |
| Small Intestine Pool | 6.4 |
| Stomach Pool | 10.9 |
| Bone Marrow Pool | 9.6 |
| Fetal Heart | 7.2 |
| Heart Pool | 4.8 |
| Lymph Node Pool | 13.5 |
| Fetal Skeletal Muscle | 67.8 |
| Skeletal Muscle Pool | 25.7 |
| Spleen Pool | 2.9 |
| Thymus Pool | 9.9 |
| CNS cancer (glio/astro) U87-MG | 10.2 |
| CNS cancer (glio/astro) U-118-MG | 24.7 |
| CNS cancer (neuro; met) SK-N-AS | 14.0 |
| CNS cancer (astro) SF-539 | 9.4 |
| CNS cancer (astro) SNB-75 | 39.2 |
| CNS cancer (glio) SNB-19 | 1.4 |
| CNS cancer (glio) SF-295 | 4.6 |
| Brain (Amygdala) Pool | 8.5 |
| Brain (cerebellum) | 6.7 |
| Brain (fetal) | 62.0 |
| Brain (Hippocampus) Pool | 19.5 |
| Cerebral Cortex Pool | 8.7 |
| Brain (Substantia nigra) Pool | 10.2 |
| Brain (Thalamus) Pool | 12.8 |
| Brain (whole) | 11.3 |
| Spinal Cord Pool | 20.9 |
| Adrenal Gland | 32.8 |
| Pituitary gland Pool | 2.7 |
| Salivary Gland | 0.5 |
| Thyroid (female) | 6.9 |
| Pancreatic ca. CAPAN2 | 100.0 |
| Pancreas Pool | 5.3 |

Column A - Rel. Exp. (%) Ag902, Run 214145283

TABLE JD
HASS Panel v1.0

| Tissue Name | A |
|---|---|
| MCF-7 C1 | 0.1 |
| MCF-7 C2 | 0.1 |
| MCF-7 C3 | 0.1 |
| MCF-7 C4 | 0.1 |
| MCF-7 C5 | 0.1 |
| MCF-7 C6 | 0.1 |
| MCF-7 C7 | 0.1 |
| MCF-7 C9 | 0.0 |
| MCF-7 C10 | 0.1 |
| MCF-7 C11 | 0.1 |
| MCF-7 C12 | 0.2 |
| MCF-7 C13 | 0.2 |
| MCF-7 C15 | 0.0 |
| MCF-7 C16 | 0.2 |
| MCF-7 C17 | 0.2 |
| T24 D1 | 0.0 |
| T24 D2 | 0.0 |
| T24 D3 | 0.1 |
| T24 D4 | 0.0 |
| T24 D5 | 0.0 |
| T24 D6 | 0.1 |
| T24 D7 | 0.1 |
| T24 D9 | 0.2 |
| T24 D10 | 0.0 |
| T24 D11 | 0.2 |
| T24 D12 | 0.1 |
| T24 D13 | 0.0 |
| T24 D15 | 0.0 |
| T24 D16 | 0.1 |
| T24 D17 | 0.4 |
| CAPaN B1 | 70.7 |
| CAPaN B2 | 45.1 |
| CAPaN B3 | 23.3 |
| CAPaN B4 | 31.6 |
| CAPaN B5 | 67.4 |
| CAPaN B6 | 50.3 |
| CAPaN B7 | 34.2 |
| CAPaN B8 | 69.3 |
| CAPaN B9 | 78.5 |
| CAPaN B10 | 47.0 |
| CAPaN B11 | 79.0 |
| CAPaN B12 | 45.1 |
| CAPaN B13 | 38.7 |
| CAPaN B14 | 42.0 |
| CAPaN B15 | 45.1 |
| CAPaN B16 | 52.9 |
| CAPaN B17 | 52.9 |
| U87-MG F1 (B) | 0.4 |
| U87-MG F2 | 0.4 |
| U87-MG F3 | 3.3 |
| U87-MG F4 | 0.2 |
| U87-MG F5 | 2.3 |
| U87-MG F6 | 11.6 |
| U87-MG F7 | 0.9 |
| U87-MG F8 | 4.4 |
| U87-MG F9 | 0.7 |
| U87-MG F10 | 2.0 |
| U87-MG F11 | 11.4 |
| U87-MG F12 | 1.8 |
| U87-MG F13 | 3.0 |
| U87-MG F14 | 9.7 |
| U87-MG F15 | 1.5 |
| U87-MG F16 | 2.4 |
| U87-MG F17 | 3.0 |
| LnCAP A1 | 0.1 |
| LnCAP A2 | 0.1 |
| LnCAP A3 | 0.0 |
| LnCAP A4 | 0.0 |
| LnCAP A5 | 0.0 |
| LnCAP A6 | 0.0 |
| LnCAP A7 | 0.0 |
| LnCAP A8 | 0.2 |
| LnCAP A9 | 0.2 |
| LnCAP A10 | 0.0 |
| LnCAP A11 | 0.4 |
| LnCAP A12 | 0.0 |
| LnCAP A13 | 0.0 |
| LnCAP A14 | 0.2 |
| LnCAP A15 | 0.3 |
| LnCAP A16 | 0.1 |
| LnCAP A17 | 0.3 |
| Primary Astrocytes | 10.0 |
| Primary Renal Proximal Tubule Epithelial cell A2 | 10.2 |
| Primary melanocytes A5 | 0.0 |
| 126443 - 341 medullo | 0.5 |
| 126444 - 487 medullo | 19.6 |
| 126445 - 425 medullo | 7.5 |
| 126446 - 690 medullo | 14.7 |
| 126447 - 54 adult glioma | 10.4 |
| 126448 - 245 adult glioma | 3.6 |
| 126449 - 317 adult glioma | 100.0 |
| 126450 - 212 glioma | 52.1 |
| 126451 - 456 glioma | 23.0 |

Column A - Rel. Exp. (%) Ag902, Run 273142965

TABLE JE
Panel 2D

| Tissue Name | A | B |
|---|---|---|
| Normal Colon | 24.1 | 28.9 |
| CC Well to Mod Diff (ODO3866) | 4.1 | 5.0 |
| CC Margin (ODO3866) | 5.9 | 3.6 |
| CC Gr.2 rectosigmoid (ODO3868) | 4.6 | 4.0 |
| CC Margin (ODO3868) | 1.3 | 1.2 |
| CC Mod Diff (ODO3920) | 19.6 | 19.5 |
| CC Margin (ODO3920) | 6.2 | 9.3 |
| CC Gr.2 ascend colon (ODO3921) | 25.9 | 34.2 |
| CC Margin (ODO3921) | 6.7 | 7.2 |
| CC from Partial Hepatectomy (ODO4309) Mets | 3.3 | 2.6 |
| Liver Margin (ODO4309) | 0.8 | 0.6 |
| Colon mets to lung (OD04451-01) | 5.0 | 3.2 |
| Lung Margin (OD04451-02) | 0.6 | 0.0 |
| Normal Prostate 6546-1 | 4.8 | 3.0 |
| Prostate Cancer (OD04410) | 3.5 | 3.9 |
| Prostate Margin (OD04410) | 6.0 | 6.8 |
| Prostate Cancer (OD04720-01) | 0.9 | 1.2 |
| Prostate Margin (OD04720-02) | 6.3 | 2.4 |
| Normal Lung 061010 | 16.5 | 18.6 |
| Lung Met to Muscle (ODO4286) | 31.4 | 28.1 |
| Muscle Margin (ODO4286) | 53.6 | 66.4 |
| Lung Malignant Cancer (OD03126) | 8.7 | 10.0 |
| Lung Margin (OD03126) | 8.8 | 4.6 |
| Lung Cancer (OD04404) | 7.2 | 9.2 |
| Lung Margin (OD04404) | 6.0 | 7.9 |
| Lung Cancer (OD04565) | 4.6 | 4.3 |
| Lung Margin (OD04565) | 1.7 | 0.9 |
| Lung Cancer (OD04237-01) | 22.4 | 16.8 |
| Lung Margin (OD04237-02) | 1.5 | 1.9 |
| Ocular Mel Met to Liver (ODO4310) | 1.4 | 1.4 |
| Liver Margin (ODO4310) | 0.1 | 0.2 |
| Melanoma Mets to Lung (OD04321) | 0.3 | 0.6 |
| Lung Margin (OD04321) | 1.2 | 2.1 |
| Normal Kidney | 3.6 | 4.7 |
| Kidney Ca, Nuclear grade 2 (OD04338) | 78.5 | 72.2 |
| Kidney Margin (OD04338) | 4.2 | 6.0 |
| Kidney Ca Nuclear grade 1/2 (OD04339) | 62.0 | 54.3 |
| Kidney Margin (OD04339) | 3.0 | 2.9 |
| Kidney Ca, Clear cell type (OD04340) | 0.5 | 1.2 |
| Kidney Margin (OD04340) | 3.5 | 3.7 |
| Kidney Ca, Nuclear grade 3 (OD04348) | 7.5 | 9.5 |
| Kidney Margin (OD04348) | 3.1 | 4.2 |
| Kidney Cancer (OD04622-01) | 100.0 | 100.0 |
| Kidney Margin (OD04622-03) | 0.0 | 0.6 |
| Kidney Cancer (OD04450-01) | 11.2 | 12.7 |
| Kidney Margin (OD04450-03) | 2.0 | 3.2 |
| Kidney Cancer 8120607 | 7.1 | 6.7 |
| Kidney Margin 8120608 | 0.5 | 0.8 |
| Kidney Cancer 8120613 | 1.6 | 0.9 |
| Kidney Margin 8120614 | 1.5 | 1.2 |
| Kidney Cancer 9010320 | 90.8 | 88.3 |
| Kidney Margin 9010321 | 5.1 | 4.1 |
| Normal Uterus | 9.5 | 6.7 |
| Uterus Cancer 064011 | 20.9 | 18.9 |
| Normal Thyroid | 42.9 | 41.2 |
| Thyroid Cancer 064010 | 11.8 | 16.6 |
| Thyroid Cancer A302152 | 12.8 | 9.9 |
| Thyroid Margin A302153 | 9.5 | 14.4 |
| Normal Breast | 16.5 | 18.9 |
| Breast Cancer (OD04566) | 2.5 | 4.0 |
| Breast Cancer (OD04590-01) | 5.7 | 3.1 |
| Breast Cancer Mets (OD04590-03) | 8.2 | 6.9 |
| Breast Cancer Metastasis (OD04655-05) | 5.4 | 3.4 |
| Breast Cancer 064006 | 11.3 | 8.4 |
| Breast Cancer 1024 | 28.7 | 34.4 |
| Breast Cancer 9100266 | 11.1 | 12.2 |
| Breast Margin 9100265 | 21.9 | 27.9 |
| Breast Cancer A209073 | 27.2 | 19.5 |
| Breast Margin A209073 | 20.4 | 17.1 |
| Normal Liver | 0.4 | 0.3 |
| Liver Cancer 064003 | 0.3 | 0.8 |
| Liver Cancer 1025 | 0.0 | 0.0 |
| Liver Cancer 1026 | 2.8 | 3.2 |
| Liver Cancer 6004-T | 0.2 | 0.4 |
| Liver Tissue 6004-N | 2.1 | 0.3 |
| Liver Cancer 6005-T | 1.1 | 3.9 |
| Liver Tissue 6005-N | 0.5 | 0.0 |
| Normal Bladder | 13.1 | 10.7 |
| Bladder Cancer 1023 | 1.2 | 2.5 |
| Bladder Cancer A302173 | 3.5 | 4.7 |
| Bladder Cancer (OD04718-01) | 6.1 | 7.6 |
| Bladder Normal Adjacent (OD04718-03) | 33.9 | 24.7 |
| Normal Ovary | 43.5 | 48.0 |
| Ovarian Cancer 064008 | 32.1 | 26.4 |
| Ovarian Cancer (OD04768-07) | 4.6 | 3.3 |
| Ovary Margin (OD04768-08) | 5.7 | 4.7 |
| Normal Stomach | 13.6 | 16.7 |
| Gastric Cancer 9060358 | 12.1 | 16.5 |
| Stomach Margin 9060359 | 8.5 | 9.9 |
| Gastric Cancer 9060395 | 14.5 | 12.9 |
| Stomach Margin 9060394 | 14.1 | 14.1 |
| Gastric Cancer 9060397 | 6.3 | 8.2 |
| Stomach Margin 9060396 | 3.0 | 3.3 |
| Gastric Cancer 064005 | 11.9 | 6.4 |

Column A - Rel. Exp. (%) Ag902, Run 146087175
Column B - Rel. Exp. (%) Ag902, Run 151091474

TABLE JF
Panel 3D

| Tissue Name | A |
|---|---|
| Daoy- Medulloblastoma | 4.8 |
| TE671- Medulloblastoma | 39.8 |
| D283 Med- Medulloblastoma | 49.3 |
| PFSK-1- Primitive Neuroectodermal | 0.0 |
| XF-498- CNS | 0.0 |
| SNB-78- Glioma | 12.6 |
| SF-268- Glioblastoma | 0.8 |
| T98G- Glioblastoma | 0.0 |
| SK-N-SH- Neuroblastoma (metastasis) | 0.5 |
| SF-295- Glioblastoma | 1.2 |
| Cerebellum | 8.1 |
| Cerebellum | 4.4 |
| NCI-H292- Mucoepidermoid lung carcinoma | 0.7 |
| DMS-114- Small cell lung cancer | 9.7 |
| DMS-79- Small cell lung cancer | 24.5 |
| NCI-H146- Small cell lung cancer | 2.2 |
| NCI-H526- Small cell lung cancer | 11.4 |
| NCI-N417- Small cell lung cancer | 5.3 |
| NCI-H82- Small cell lung cancer | 13.1 |
| NCI-H157- Squamous cell lung cancer (metastasis) | 0.0 |
| NCI-H1155- Large cell lung cancer | 16.2 |
| NCI-H1299- Large cell lung cancer | 2.4 |
| NCI-H727- Lung carcinoid | 1.7 |
| NCI-UMC-11- Lung carcinoid | 3.1 |
| LX-1- Small cell lung cancer | 5.1 |
| Colo-205- Colon cancer | 1.6 |
| KM12- Colon cancer | 0.0 |
| KM20L2- Colon cancer | 3.4 |
| NCI-H716- Colon cancer | 100.0 |
| SW-48- Colon adenocarcinoma | 0.0 |
| SW1116- Colon adenocarcinoma | 0.0 |
| LS 174T- Colon adenocarcinoma | 8.8 |
| SW-948- Colon adenocarcinoma | 0.0 |
| SW-480- Colon adenocarcinoma | 0.0 |
| NCI-SNU-5- Gastric carcinoma | 5.4 |
| KATO III- Gastric carcinoma | 1.9 |
| NCI-SNU-16- Gastric carcinoma | 10.7 |
| NCI-SNU-1- Gastric carcinoma | 0.0 |
| RF-1- Gastric adenocarcinoma | 0.7 |
| RF-48- Gastric adenocarcinoma | 1.4 |
| MKN-45- Gastric carcinoma | 0.9 |
| NCI-N87- Gastric carcinoma | 0.0 |
| OVCAR-5- Ovarian carcinoma | 1.0 |
| RL95-2- Uterine carcinoma | 0.0 |
| HelaS3- Cervical adenocarcinoma | 0.7 |
| Ca Ski- Cervical epidermoid carcinoma (metastasis) | 0.0 |
| ES-2- Ovarian clear cell carcinoma | 0.0 |
| Ramos- Stimulated with PMA/ionomycin 6h | 0.0 |
| Ramos- Stimulated with PMA/ionomycin 14h | 0.0 |
| MEG-01- Chronic myelogenous leukemia (megokaryoblast) | 2.2 |
| Raji- Burkitt's lymphoma | 0.0 |
| Daudi- Burkitt's lymphoma | 0.8 |
| U266- B-cell plasmacytoma | 0.0 |
| CA46- Burkitt's lymphoma | 1.3 |
| RL- non-Hodgkin's B-cell lymphoma | 0.0 |
| JM1- pre-B-cell lymphoma | 0.0 |
| Jurkat- T cell leukemia | 0.0 |
| TF-1 - Erythroleukemia | 0.6 |
| HUT 78- T-cell lymphoma | 0.8 |
| U937- Histiocytic lymphoma | 0.7 |
| KU-812- Myelogenous leukemia | 0.0 |
| 769-P- Clear cell renal carcinoma | 0.0 |
| Caki-2- Clear cell renal carcinoma | 5.8 |
| SW 839- Clear cell renal carcinoma | 1.7 |
| Rhabdoid kidney tumor | 25.2 |
| Hs766T- Pancreatic carcinoma (LN metastasis) | 3.9 |
| CAPAN-1- Pancreatic adenocarcinoma (liver metastasis) | 3.1 |
| SU86.86- Pancreatic carcinoma (liver metastasis) | 25.9 |
| BxPC-3- Pancreatic adenocarcinoma | 0.0 |
| HPAC- Pancreatic adenocarcinoma | 2.9 |
| MIA PaCa-2- Pancreatic carcinoma | 0.0 |
| CFPAC-1- Pancreatic ductal adenocarcinoma | 6.3 |
| PANC-1- Pancreatic epithelioid ductal carcinoma | 8.5 |
| T24- Bladder carcinma (transitional cell) | 0.0 |
| 5637- Bladder carcinoma | 0.0 |
| HT-1197- Bladder carcinoma | 0.0 |
| UM-UC-3- Bladder carcinma (transitional cell) | 0.0 |
| A204- Rhabdomyosarcoma | 0.5 |
| HT-1080- Fibrosarcoma | 3.7 |
| MG-63- Osteosarcoma | 7.9 |
| SK-LMS-1- Leiomyosarcoma (vulva) | 2.2 |
| SJRH30- Rhabdomyosarcoma (met to bone marrow) | 33.4 |
| A431- Epidermoid carcinoma | 0.0 |
| WM266-4- Melanoma | 0.8 |
| DU 145- Prostate carcinoma (brain metastasis) | 0.8 |
| MDA-MB-468- Breast adenocarcinoma | 0.0 |
| SCC-4- Squamous cell carcinoma of tongue | 1.3 |
| SCC-9- Squamous cell carcinoma of tongue | 0.0 |
| SCC-15- Squamous cell carcinoma of tongue | 0.0 |
| CAL 27- Squamous cell carcinoma of tongue | 0.0 |

Column A - Rel. Exp. (%) Ag902, Run 164844768

TABLE JG
Panel 4.1D

| Tissue Name | A |
|---|---|
| Secondary Th1 act | 0.0 |
| Secondary Th2 act | 0.0 |
| Secondary Tr1 act | 0.2 |
| Secondary Th1 rest | 0.2 |
| Secondary Th2 rest | 0.3 |
| Secondary Tr1 rest | 0.0 |
| Primary Th1 act | 0.0 |
| Primary Th2 act | 0.1 |
| Primary Tr1 act | 0.0 |
| Primary Th1 rest | 0.4 |
| Primary Th2 rest | 0.0 |
| Primary Tr1 rest | 0.0 |
| CD45RA CD4 lymphocyte act | 1.7 |
| CD45RO CD4 lymphocyte act | 0.2 |
| CD8 lymphocyte act | 0.0 |
| Secondary CD8 lymphocyte rest | 0.3 |
| Secondary CD8 lymphocyte act | 0.0 |
| CD4 lymphocyte none | 0.0 |
| 2ry Th1/Th2/Tr1_anti-CD95 CH11 | 0.3 |
| LAK cells rest | 0.0 |
| LAK cells IL-2 | 0.0 |
| LAK cells IL-2 + IL-12 | 0.0 |
| LAK cells IL-2 + IFN gamma | 0.5 |
| LAK cells IL-2 + IL-18 | 0.7 |
| LAK cells PMA/ionomycin | 0.0 |
| NK Cells IL-2 rest | 0.8 |
| Two Way MLR 3 day | 0.8 |
| Two Way MLR 5 day | 0.0 |
| Two Way MLR 7 day | 0.0 |
| PBMC rest | 0.0 |
| PBMC PWM | 0.0 |
| PBMC PHA-L | 0.0 |
| Ramos (B cell) none | 0.2 |
| Ramos (B cell) ionomycin | 0.0 |
| B lymphocytes PWM | 0.0 |
| B lymphocytes CD40L and IL-4 | 0.2 |
| EOL-1 dbcAMP | 0.0 |
| EOL-1 dbcAMP PMA/ionomycin | 0.0 |
| Dendritic cells none | 0.0 |
| Dendritic cells LPS | 100.0 |
| Dendritic cells anti-CD40 | 0.0 |
| Monocytes rest | 0.0 |
| Monocytes LPS | 0.8 |
| Macrophages rest | 6.9 |
| Macrophages LPS | 1.0 |
| HUVEC none | 0.0 |
| HUVEC starved | 0.0 |
| HUVEC IL-1beta | 0.0 |
| HUVEC IFN gamma | 1.5 |
| HUVEC TNF alpha + IFN gamma | 0.0 |
| HUVEC TNF alpha + IL4 | 0.0 |
| HUVEC IL-11 | 0.0 |
| Lung Microvascular EC none | 0.4 |
| Lung Microvascular EC TNFalpha + IL-1beta | 0.3 |
| Microvascular Dermal EC none | 0.0 |
| Microsvasular Dermal EC TNFalpha + IL-1beta | 0.0 |
| Bronchial epithelium TNFalpha + IL1beta | 2.4 |
| Small airway epithelium none | 1.6 |
| Small airway epithelium TNFalpha + IL-1beta | 0.4 |
| Coronery artery SMC rest | 0.9 |
| Coronery artery SMC TNFalpha + IL-1beta | 1.4 |
| Astrocytes rest | 13.3 |
| Astrocytes TNFalpha + IL-1beta | 9.8 |
| KU-812 (Basophil) rest | 0.0 |
| KU-812 (Basophil) PMA/ionomycin | 0.0 |
| CCD1106 (Keratinocytes) none | 0.3 |
| CCD1106 (Keratinocytes) TNFalpha + IL-1beta | 0.0 |
| Liver cirrhosis | 0.1 |
| NCI-H292 none | 0.5 |
| NCI-H292 IL-4 | 0.0 |
| NCI-H292 IL-9 | 1.0 |
| NCI-H292 IL-13 | 0.0 |
| NCI-H292 IFN gamma | 0.3 |
| HPAEC none | 0.0 |
| HPAEC TNF alpha + IL-1 beta | 0.0 |
| Lung fibroblast none | 4.2 |
| Lung fibroblast TNF alpha + IL-1 beta | 14.5 |
| Lung fibroblast IL-4 | 4.1 |
| Lung fibroblast IL-9 | 3.0 |
| Lung fibroblast IL-13 | 4.2 |
| Lung fibroblast IFN gamma | 4.2 |
| Dermal fibroblast CCD1070 rest | 3.6 |
| Dermal fibroblast CCD1070 TNF alpha | 1.5 |
| Dermal fibroblast CCD1070 IL-1 beta | 2.2 |
| Dermal fibroblast IFN gamma | 9.5 |
| Dermal fibroblast IL-4 | 30.4 |
| Dermal Fibroblasts rest | 6.1 |
| Neutrophils TNFa + LPS | 0.0 |
| Neutrophils rest | 0.0 |
| Colon | 0.9 |
| Lung | 3.1 |
| Thymus | 8.4 |
| Kidney | 1.5 |

Column A - Rel. Exp. (%) Ag902, Run 184565260

TABLE JH
Panel 5D

| Tissue Name | A |
|---|---|
| 97457_Patient-02go_adipose | 2.0 |
| 97476_Patient-07sk_skeletal muscle | 12.9 |
| 97477_Patient-07ut_uterus | 0.0 |
| 97478_Patient-07pl_placenta | 39.5 |
| 97481_Patient-08sk_skeletal muscle | 8.8 |
| 97482_Patient-08ut_uterus | 0.8 |
| 97483_Patient-08pl_placenta | 25.2 |
| 97486_Patient-09sk_skeletal muscle | 9.0 |
| 97487_Patient-09ut_uterus | 2.3 |
| 97488_Patient-09pl_placenta | 27.5 |
| 97492_Patient-10ut_uterus | 0.6 |
| 97493_Patient-10pl_placenta | 80.7 |
| 97495_Patient-11go_adipose | 2.7 |
| 97496_Patient-11sk_skeletal muscle | 47.0 |
| 97497_Patient-11ut_uterus | 0.0 |
| 97498_Patient-11pl_placenta | 57.4 |
| 97500_Patient-12go_adipose | 5.1 |
| 97501_Patient-12sk_skeletal muscle | 100.0 |
| 97502_Patient-12ut_uterus | 1.3 |
| 97503_Patient-12pl_placenta | 35.4 |
| 94721_Donor 2 U - A_Mesenchymal Stem Cells | 3.5 |
| 94722_Donor 2 U - B_Mesenchymal Stem Cells | 1.8 |
| 94723_Donor 2 U - C_Mesenchymal Stem Cells | 3.7 |
| 94709_Donor 2 AM - A_adipose | 1.5 |
| 94710_Donor 2 AM - B_adipose | 3.0 |
| 94711_Donor 2 AM - C_adipose | 3.5 |
| 94712_Donor 2 AD - A_adipose | 5.0 |
| 94713_Donor 2 AD - B_adipose | 6.6 |
| 94714_Donor 2 AD - C_adipose | 6.7 |
| 94742_Donor 3 U - A_Mesenchymal Stem Cells | 2.8 |
| 94743_Donor 3 U - B_Mesenchymal Stem Cells | 6.0 |
| 94730_Donor 3 AM - A_adipose | 3.4 |
| 94731_Donor 3 AM - B_adipose | 2.8 |
| 94732_Donor 3 AM - C_adipose | 3.1 |
| 94733_Donor 3 AD - A_adipose | 9.5 |
| 94734_Donor 3 AD - B_adipose | 3.3 |
| 94735_Donor 3 AD - C_adipose | 8.0 |
| 77138_Liver_HepG2untreated | 6.2 |
| 73556_Heart_Cardiac stromal cells (primary) | 0.0 |
| 81735_Small Intestine | 5.0 |
| 72409_Kidney_Proximal Convoluted Tubule | 2.6 |
| 82685_Small intestine_Duodenum | 1.2 |
| 90650_Adrenal_Adrenocortical adenoma | 4.4 |
| 72410_Kidney_HRCE | 12.7 |
| 72411_Kidney_HRE | 36.9 |
| 73139_Uterus_Uterine smooth muscle cells | 4.2 |

Column A - Rel. Exp. (%) Ag902, Run 258659600

CNS_neurodegeneration_v1.0 Summary: Ag902 Expression of the CG55379-01 and CG55379-04 genes was upregulated in the temporal cortex of Alzheimer's disease patients compared to normal patients. Therefore, therapeutic modulation of the activity of these genes or their protein products using nucleic acid, protein, antibody and small molecule drugs is useful decreasing neuronal death that accompanies Alzheimer's disease.
General_screening_panel_v1.4 Summary: Ag902 Highest expression of these genes was detected in pancreatic cancer cell line CAPAN2 (CT=27). Moderate levels of expression of these genes were also seen in cluster of cell lines derived from gastric, colon, lung, renal, breast, ovarian, prostate, and brain cancers and melanomas. Gene or protein expression levels are useful as a marker to detect the presence of these cancers. Therapeutic modulation of the activity of these genes using nucleic acid, protein, antibody or small molecule drugs will be effective in the treatment of pancreatic, gastric, colon, lung, renal, breast, ovarian, prostate, melanoma and brain cancers.

Among tissues with metabolic or endocrine function, these genes were expressed at moderate levels in pancreas, adipose, adrenal gland, thyroid, pituitary gland, skeletal muscle, heart, fetal liver and the gastrointestinal tract. Therapeutic modulation of the activity of these genes or their protein products is useful in the treatment of endocrine/metabolically related diseases, such as obesity and diabetes.

In addition, these gene variants were expressed at moderate levels in all regions of the central nervous system examined, including amygdala, hippocampus, substantia nigra, thalamus, cerebellum, cerebral cortex, and spinal cord. The protein encoded by this gene is a homolog of mouse NOPE protein, which functions as a guidance receptor in the developing CNS (Salbaum J M, Kappen C., 2000, Cloning and expression of nope, a new mouse gene of the immunoglobulin superfamily related to guidance receptors. Genomics. 64(l):15-23, PMID: 10708514). Therapeutic modulation of the activity of these genes or their protein products is useful in the treatment of central nervous system disorders such as Alzheimer's disease, Parkinson's disease, epilepsy, multiple sclerosis, schizophrenia and depression.

Expression of these genes was higher in fetal tissues relative to adult tissues, especially in fetal liver, lung, and brain. The relative overexpression of these genes in fetal tissue indicates that the encoded proteins may enhance liver, lung and brain growth or development in the fetus and thus may also act in a regenerative capacity in the adult. Therefore, therapeutic modulation of these genes and/or encoded proteins is useful in treatment of liver, lung and brain related diseases.

HASS Panel v1.0 Summary: Ag902 Highest expression of the CG55379-01 and CG55379-04 genes was detected in glioma (CT=28). Moderate to low levels of expression of these variants were also seen in pancreatic cancer cell line CAPaN and glioblastoma/astrocytoma cell lines. The expression of these genes was not altered by oxygen deprivation, acidic conditions or a serum-starved environment. Therapeutic modulation of the activity of these variants are useful in the treatment of pancreatic cancer, medulloblastoma and glioma.

Panel 2D Summary: Ag902 Highest expression of these genes was detected in a kidney cancer sample (CTs=30). The CG55379-01 and CG55379-04 genes were overexpressed in 7/9 kidney cancer and 2/4 colon cancer samples relative to the corresponding normal adjacent tissues. Gene or protein expression levels are useful in the diagnosis of kidney and colon cancer. Therapeutic modulation of the activity of these variants or their protein products using nucleic acid, protein, antibody and small molecule drugs are useful in the treatment of kidney cancer.

Panel 3D Summary: Ag902 Highest expression of the CG55379-01 and CG55379-04 genes was detected in a colon cancer cell line (CT=30). These variants were also expressed in cancer cell lines derived from kidney, lung, brain, pancreas and bone cancers. This observation indicates a possible role for this gene in the pathogenesis of these cancers. Please see panel 2D for further discussion of this gene.

Panel 4.1D Summary: Ag902 Highest expression of these genes was detected in activated dendritic cells (CT=30). The expression of these variants was also induced in LPS-stimulated dendritic cells and in IL-4-stimulated dermal fibroblasts. Low expression of this gene was also seen in astrocytes and normal thymus. The CG55379-01 and CG55379-04 genes encodes variants homologous to the mouse NOPE protein, a guidance receptors (Salbaum J M, Kappen C., 2000, Cloning and expression of nope, a new mouse gene of the immunoglobulin superfamily related to guidance receptors. Genomics. 64(1):15-23, PMID: 10708514). These proteins may act as a receptor for dendritic cells and dermal fibroblasts and may control interactions between these cells and other cell types during antigen presentation or apoptosis similar to netrins (Livesey F. J., 1999, Netrins and netrin receptors. Cell Mol. Life Sci. 56: 62-68, PMID: 11213262). Therapeutic modulation of the activity of these variants or their protein products are useful in blocking inflammation in diseases such as asthma, arthritis, psoriasis, allergy and other diseases in which dendritic cell or dermal fibroblasts play important roles.

Panel 5D Summary: Ag902 Highest expression of these genes was seen in placenta of a diabetic patient on insulin (CT=32.3). Significant expression of these genes were also seen in placenta from diabetic and non-diabetic patients. Please see panel 1.4 for further discussion of this gene.

K. CG55688-01: Cyr61

Expression of gene CG55688-01 was assessed using the primer-probe set Ag1148, described in Table KA. Results of the RTQ-PCR runs are shown in Tables KB, KC, KD, KE, KF and KG.

TABLE KA
Probe Name Ag1148

| | | | Start | SEQ ID |
|---|---|---|---|---|
| Primers | Sequences | Length | Position | No |
| Forward | 5′-gtgtctgtgagaggcagc | 22 | 1683 | 319 |
| | tatc-3′ | | | |
| Probe | TET-5′-tgcactctaaactg | 29 | 1705 | 320 |
| | caaacagaaatca | | | |
| | gg-3′-TAMRA | | | |
| Reverse | 5′-ccccaaaagctacatttt | 22 | 1758 | 321 |
| | gata-3′ | | | |

TABLE KB
HASS Panel v1.0

| Tissue Name | A |
|---|---|
| MCF-7 C1 | 1.4 |
| MCF-7 C2 | 0.8 |
| MCF-7 C3 | 0.3 |
| MCF-7 C4 | 0.7 |
| MCF-7 C5 | 0.4 |
| MCF-7 C6 | 0.8 |
| MCF-7 C7 | 0.3 |
| MCF-7 C9 | 1.0 |
| MCF-7 C10 | 0.3 |
| MCF-7 C11 | 0.1 |
| MCF-7 C12 | 0.5 |
| MCF-7 C13 | 0.3 |
| MCF-7 C15 | 0.7 |
| MCF-7 C16 | 3.7 |
| MCF-7 C17 | 2.4 |
| T24 D1 | 65.5 |
| T24 D2 | 47.3 |
| T24 D3 | 34.2 |
| T24 D4 | 97.9 |
| T24 D5 | 38.7 |
| T24 D6 | 100.0 |
| T24 D7 | 52.1 |
| T24 D9 | 33.2 |
| T24 D10 | 20.7 |
| T24 D11 | 35.8 |
| T24 D12 | 92.7 |
| T24 D13 | 29.3 |
| T24 D15 | 67.8 |
| T24 D16 | 74.7 |
| T24 D17 | 62.9 |
| CAPaN B1 | 1.7 |
| CAPaN B2 | 1.2 |
| CAPaN B3 | 0.3 |
| CAPaN B4 | 0.7 |
| CAPaN B5 | 0.6 |
| CAPaN B6 | 2.7 |
| CAPaN B7 | 1.2 |
| CAPaN B8 | 0.3 |
| CAPaN B9 | 2.1 |
| CAPaN B10 | 2.4 |
| CAPaN B11 | 1.1 |
| CAPaN B12 | 3.4 |
| CAPaN B13 | 2.3 |
| CAPaN B14 | 0.4 |
| CAPaN B15 | 2.7 |
| CAPaN B16 | 3.7 |
| CAPaN B17 | 6.7 |
| U87-MG F1 (B) | 0.3 |
| U87-MG F2 | 0.9 |
| U87-MG F3 | 0.5 |
| U87-MG F4 | 1.8 |
| U87-MG F5 | 21.8 |
| U87-MG F6 | 1.3 |
| U87-MG F7 | 1.8 |
| U87-MG F8 | 0.2 |
| U87-MG F9 | 5.0 |
| U87-MG F10 | 4.0 |
| U87-MG F11 | 1.4 |
| U87-MG F12 | 20.3 |
| U87-MG F13 | 2.8 |
| U87-MG F14 | 0.4 |
| U87-MG F15 | 16.7 |
| U87-MG F16 | 25.7 |
| U87-MG F17 | 34.2 |
| LnCAP A1 | 1.4 |
| LnCAP A2 | 0.7 |
| LnCAP A3 | 0.5 |
| LnCAP A4 | 0.7 |
| LnCAP A5 | 0.4 |
| LnCAP A6 | 1.3 |
| LnCAP A7 | 0.4 |
| LnCAP A8 | 0.6 |
| LnCAP A9 | 0.3 |
| LnCAP A10 | 1.2 |
| LnCAP A11 | 1.2 |
| LnCAP A12 | 0.2 |
| LnCAP A13 | 0.4 |
| LnCAP A14 | 0.1 |
| LnCAP A15 | 0.1 |
| LnCAP A16 | 0.7 |
| LnCAP A17 | 0.6 |
| Primary Astrocytes | 52.1 |
| Primary Renal Proximal Tubule Epithelial cell A2 | 14.0 |
| Primary melanocytes A5 | 25.2 |
| 126443 - 341 medullo | 0.1 |
| 126444 - 487 medullo | 1.7 |
| 126445 - 425 medullo | 1.7 |
| 126446 - 690 medullo | 0.4 |
| 126447 - 54 adult glioma | 29.7 |
| 126448 - 245 adult glioma | 2.6 |
| 126449 - 317 adult glioma | 2.4 |
| 126450 - 212 glioma | 6.8 |
| 126451 - 456 glioma | 0.3 |

Column A - Rel. Exp. (%) Ag1148, Run 268362647

TABLE KC
PGI1.0

| Tissue Name | A |
|---|---|
| 162191_Normal Lung 1 (IBS) | 0.8 |
| 160468_MD lung | 1.3 |
| 156629_MD Lung 13 | 0.3 |
| 162570_Normal Lung 4 (Aastrand) | 0.8 |
| 162571_Normal Lung 3 (Aastrand) | 0.0 |
| 162187_Fibrosis Lung 2 (Genomic Collaborative) | 9.3 |
| 151281_Fibrosis lung 11(Ardais) | 78.5 |
| 162186_Fibrosis Lung 1 (Genomic Collaborative) | 93.3 |
| 162190_Asthma Lung 4 (Genomic Collaborative) | 17.8 |
| 160467_Asthma Lung 13 (MD) | 0.7 |
| 137027_Emphysema Lung 1 (Ardais) | 0.8 |
| 137028_Emphysema Lung 2 (Ardais) | 3.5 |
| 137040_Emphysema Lung 3 (Ardais) | 24.1 |
| 137041_Emphysema Lung 4 (Ardais) | 3.4 |
| 137043_Emphysema Lung 5 (Ardais) | 12.5 |
| 142817_Emphysema Lung 6 (Ardais) | 34.2 |
| 142818_Emphysema Lung 7 (Ardais) | 16.7 |
| 142819_Emphysema Lung 8 (Ardais) | 36.9 |
| 142820_Emphysema Lung 9 (Ardais) | 3.2 |
| 142821_Emphysema Lung 10 (Ardais) | 21.0 |
| 162185_Emphysema Lung 12 (Ardais) | 73.7 |
| 162184_Emphysema Lung 13 (Ardais) | 44.4 |
| 162183_Emphysema Lung 14 (Ardais) | 100.0 |
| 162188_Emphysema Lung 15 (Genomic Collaborative) | 65.1 |
| 162177_NAT UC Colon 1 (Ardais) | 4.9 |
| 162176_UC Colon 1 (Ardais) | 8.1 |
| 162179_NAT UC Colon 2(Ardais) | 4.4 |
| 162178_UC Colon 2(Ardais) | 36.3 |
| 162181_NAT UC Colon 3(Ardais) | 31.6 |
| 162180_UC Colon 3(Ardais) | 24.5 |
| 162182_NAT UC Colon 4 (Ardais) | 2.7 |
| 137042_UC Colon 1108 | 4.8 |
| 137029_UC Colon 8215 | 14.4 |
| 137031_UC Colon 8217 | 33.9 |
| 137036_UC Colon 1137 | 10.2 |
| 137038_UC Colon 1491 | 43.2 |
| 137039_UC Colon 1546 | 92.0 |
| 162593_Crohn's 47751 (NDRI) | 0.2 |
| 162594_NAT Crohn's 47751 (NDRI) | 0.9 |

Column A - Rel. Exp. (%) Ag1148, Run 398125354

TABLE KD
Panel 1.3D

| Tissue Name | A |
|---|---|
| Liver adenocarcinoma | 4.5 |
| Pancreas | 1.8 |
| Pancreatic ca. CAPAN 2 | 0.6 |
| Adrenal gland | 6.3 |
| Thyroid | 11.2 |
| Salivary gland | 1.5 |
| Pituitary gland | 1.3 |
| Brain (fetal) | 0.8 |
| Brain (whole) | 1.4 |
| Brain (amygdala) | 0.8 |
| Brain (cerebellum) | 0.1 |
| Brain (hippocampus) | 5.9 |
| Brain (substantia nigra) | 1.7 |
| Brain (thalamus) | 1.2 |
| Cerebral Cortex | 1.5 |
| Spinal cord | 2.0 |
| glio/astro U87-MG | 0.7 |
| glio/astro U-118-MG | 19.6 |
| astrocytoma SW1783 | 21.8 |
| neuro*; met SK-N-AS | 0.8 |
| astrocytoma SF-539 | 16.3 |
| astrocytoma SNB-75 | 11.3 |
| glioma SNB-19 | 8.4 |
| glioma U251 | 10.2 |
| glioma SF-295 | 5.6 |
| Heart (fetal) | 28.9 |
| Heart | 9.5 |
| Skeletal muscle (fetal) | 14.1 |
| Skeletal muscle | 3.3 |
| Bone marrow | 1.7 |
| Thymus | 1.2 |
| Spleen | 12.6 |
| Lymph node | 21.3 |
| Colorectal | 8.6 |
| Stomach | 5.2 |
| Small intestine | 9.9 |
| Colon ca. SW480 | 2.6 |
| Colon ca.* SW620(SW480 met) | 0.1 |
| Colon ca. HT29 | 0.1 |
| Colon ca. HCT-116 | 1.0 |
| Colon ca. CaCo-2 | 0.7 |
| Colon ca. tissue(ODO3866) | 9.8 |
| Colon ca. HCC-2998 | 0.6 |
| Gastric ca.* (liver met) NCI-N87 | 3.0 |
| Bladder | 2.5 |
| Trachea | 17.9 |
| Kidney | 1.8 |
| Kidney (fetal) | 9.9 |
| Renal ca. 786-0 | 13.0 |
| Renal ca. A498 | 11.1 |
| Renal ca. RXF 393 | 20.2 |
| Renal ca. ACHN | 17.2 |
| Renal ca. UO-31 | 26.1 |
| Renal ca. TK-10 | 10.2 |
| Liver | 1.0 |
| Liver (fetal) | 8.0 |
| Liver ca. (hepatoblast) HepG2 | 0.9 |
| Lung | 45.4 |
| Lung (fetal) | 34.9 |
| Lung ca. (small cell) LX-1 | 0.1 |
| Lung ca. (small cell) NCI-H69 | 0.0 |
| Lung ca. (s. cell var.) SHP-77 | 0.0 |
| Lung ca. (large cell)NCI-H460 | 0.2 |
| Lung ca. (non-sm. cell) A549 | 0.2 |
| Lung ca. (non-s. cell) NCI-H23 | 2.8 |
| Lung ca. (non-s. cell) HOP-62 | 1.9 |
| Lung ca. (non-s. cl) NCI-H522 | 12.2 |
| Lung ca. (squam.) SW 900 | 2.7 |
| Lung ca. (squam.) NCI-H596 | 0.0 |
| Mammary gland | 45.4 |
| Breast ca.* (pl. ef) MCF-7 | 0.1 |
| Breast ca.* (pl. ef) MDA-MB-231 | 16.7 |
| Breast ca.* (pl. ef) T47D | 1.3 |
| Breast ca. BT-549 | 11.1 |
| Breast ca. MDA-N | 0.3 |
| Ovary | 16.3 |
| Ovarian ca. OVCAR-3 | 1.6 |
| Ovarian ca. OVCAR-4 | 12.7 |
| Ovarian ca. OVCAR-5 | 1.9 |
| Ovarian ca. OVCAR-8 | 12.6 |
| Ovarian ca. IGROV-1 | 1.8 |
| Ovarian ca.* (ascites) SK-OV-3 | 3.5 |
| Uterus | 11.0 |
| Placenta | 9.0 |
| Prostate | 5.7 |
| Prostate ca.* (bone met)PC-3 | 1.9 |
| Testis | 3.8 |
| Melanoma Hs688(A).T | 100.0 |
| Melanoma* (met) Hs688(B).T | 88.3 |
| Melanoma UACC-62 | 0.2 |
| Melanoma M14 | 0.1 |
| Melanoma LOX IMVI | 3.1 |
| Melanoma* (met) SK-MEL-5 | 0.1 |
| Adipose | 45.4 |

Column A - Rel. Exp. (%) Ag1148, Run 151759893

TABLE KE
Panel 2D
Column A - Rel. Exp. (%) Ag1148, Run 145375638
Column B - Rel. Exp. (%) Ag1148, Run 147104767

| Tissue Name | A | B |
|---|---|---|
| Normal Colon | 5.0 | 15.7 |
| CC Well to Mod Diff (ODO3866) | 3.9 | 14.6 |
| CC Margin (ODO3866) | 9.0 | 23.7 |
| CC Gr. 2 rectosigmoid (ODO3868) | 0.7 | 1.7 |
| CC Margin (ODO3868) | 3.3 | 5.6 |
| CC Mod Diff (ODO3920) | 0.4 | 1.0 |
| CC Margin (ODO3920) | 3.0 | 5.9 |
| CC Gr. 2 ascend colon (ODO3921) | 67.4 | 14.3 |
| CC Margin (ODO3921) | 3.5 | 11.3 |
| CC from Partial Hepatectomy | 2.3 | 6.0 |
| (ODO4309) Mets | | |
| Liver Margin (ODO4309) | 0.9 | 3.5 |
| Colon mets to lung (OD04451-01) | 4.2 | 3.9 |
| Lung Margin (OD04451-02) | 1.7 | 3.5 |
| Normal Prostate 6546-1 | 4.9 | 5.1 |
| Prostate Cancer (OD04410) | 21.9 | 29.9 |
| Prostate Margin (OD04410) | 39.2 | 30.8 |
| Prostate Cancer (OD04720-01) | 9.7 | 8.6 |
| Prostate Margin (OD04720-02) | 49.3 | 44.4 |
| Normal Lung 061010 | 19.3 | 21.5 |
| Lung Met to Muscle (ODO4286) | 2.8 | 2.6 |
| Muscle Margin (ODO4286) | 12.3 | 8.0 |
| Lung Malignant Cancer (OD03126) | 10.0 | 1.0 |
| Lung Margin (OD03126) | 17.6 | 32.3 |
| Lung Cancer (OD04404) | 4.7 | 11.7 |
| Lung Margin (OD04404) | 3.0 | 7.2 |
| Lung Cancer (OD04565) | 4.0 | 2.8 |
| Lung Margin (OD04565) | 11.8 | 4.7 |
| Lung Cancer (OD04237-01) | 5.1 | 5.1 |
| Lung Margin (OD04237-02) | 7.2 | 21.6 |
| Ocular Mel Met to Liver (ODO4310) | 1.2 | 0.7 |
| Liver Margin (ODO4310) | 6.9 | 5.2 |
| Melanoma Mets to Lung (OD04321) | 3.0 | 2.9 |
| Lung Margin (OD04321) | 33.2 | 37.4 |
| Normal Kidney | 11.2 | 21.5 |
| Kidney Ca, Nuclear grade 2 | 11.8 | 17.2 |
| (OD04338) | | |
| Kidney Margin (OD04338) | 17.8 | 34.6 |
| Kidney Ca Nuclear grade ½ | 2.6 | 5.2 |
| (OD04339) | | |
| Kidney Margin (OD04339) | 15.8 | 15.4 |
| Kidney Ca, Clear cell type (OD04340) | 100.0 | 59.0 |
| Kidney Margin (OD04340) | 36.1 | 57.0 |
| Kidney Ca, Nuclear grade 3 | 2.6 | 2.0 |
| (OD04348) | | |
| Kidney Margin (OD04348) | 25.3 | 14.5 |
| Kidney Cancer (OD04622-01) | 32.8 | 15.5 |
| Kidney Margin (OD04622-03) | 6.2 | 3.7 |
| Kidney Cancer (OD04450-01) | 12.5 | 9.1 |
| Kidney Margin (OD04450-03) | 19.5 | 14.8 |
| Kidney Cancer 8120607 | 3.8 | 2.9 |
| Kidney Margin 8120608 | 6.3 | 6.6 |
| Kidney Cancer 8120613 | 0.6 | 0.7 |
| Kidney Margin 8120614 | 1.1 | 4.0 |
| Kidney Cancer 9010320 | 4.3 | 14.6 |
| Kidney Margin 9010321 | 4.7 | 6.7 |
| Normal Uterus | 33.2 | 25.5 |
| Uterus Cancer 064011 | 72.7 | 44.1 |
| Normal Thyroid | 8.6 | 11.2 |
| Thyroid Cancer 064010 | 8.4 | 5.0 |
| Thyroid Cancer A302152 | 5.5 | 4.9 |
| Thyroid Margin A302153 | 40.1 | 35.4 |
| Normal Breast | 25.3 | 23.2 |
| Breast Cancer (OD04566) | 4.2 | 2.5 |
| Breast Cancer (OD04590-01) | 1.5 | 3.8 |
| Breast Cancer Mets | 16.4 | 9.7 |
| (OD04590-03) | | |
| Breast Cancer Metastasis | 3.1 | 2.3 |
| (OD04655-05) | | |
| Breast Cancer 064006 | 5.0 | 5.3 |
| Breast Cancer 1024 | 1.6 | 5.1 |
| Breast Cancer 9100266 | 5.2 | 3.9 |
| Breast Margin 9100265 | 6.7 | 5.1 |
| Breast Cancer A209073 | 8.8 | 5.6 |
| Breast Margin A209073 | 1.0 | 1.8 |
| Normal Liver | 0.6 | 0.6 |
| Liver Cancer 064003 | 0.2 | 0.8 |
| Liver Cancer 1025 | 1.6 | 4.7 |
| Liver Cancer 1026 | 1.4 | 3.6 |
| Liver Cancer 6004-T | 2.2 | 4.1 |
| Liver Tissue 6004-N | 0.3 | 2.1 |
| Liver Cancer 6005-T | 2.0 | 4.4 |
| Liver Tissue 6005-N | 0.8 | 1.8 |
| Normal Bladder | 6.2 | 8.9 |
| Bladder Cancer 1023 | 3.8 | 4.6 |
| Bladder Cancer A302173 | 0.8 | 2.4 |
| Bladder Cancer (OD04718-01) | 6.2 | 9.7 |
| Bladder Normal Adjacent | 50.0 | 100.0 |
| (OD04718-03) | | |
| Normal Ovary | 1.6 | 7.5 |
| Ovarian Cancer 064008 | 26.1 | 69.3 |
| Ovarian Cancer (OD04768-07) | 7.7 | 18.0 |
| Ovary Margin (OD04768-08) | 75.8 | 62.0 |
| Normal Stomach | 2.6 | 8.0 |
| Gastric Cancer 9060358 | 0.5 | 1.5 |
| Stomach Margin 9060359 | 2.5 | 7.0 |
| Gastric Cancer 9060395 | 1.5 | 6.0 |
| Stomach Margin 9060394 | 2.1 | 8.7 |
| Gastric Cancer 9060397 | 4.4 | 16.7 |
| Stomach Margin 9060396 | 0.6 | 1.9 |
| Gastric Cancer 064005 | 2.0 | 7.0 |

TABLE KF
Panel 3D

| Tissue Name | A |
|---|---|
| Daoy- Medulloblastoma | 15.3 |
| TE671- Medulloblastoma | 0.0 |
| D283 Med- Medulloblastoma | 1.5 |
| PFSK-1- Primitive Neuroectodermal | 3.4 |
| XF-498- CNS | 22.1 |
| SNB-78- Glioma | 25.2 |
| SF-268- Glioblastoma | 60.3 |
| T98G- Glioblastoma | 19.2 |
| SK-N-SH- Neuroblastoma (metastasis) | 13.0 |
| SF-295- Glioblastoma | 3.5 |
| Cerebellum | 0.3 |
| Cerebellum | 0.1 |
| NCI-H292- Mucoepidermoid lung carcinoma | 100.0 |
| DMS-114- Small cell lung cancer | 2.7 |
| DMS-79- Small cell lung cancer | 0.2 |
| NCI-H146- Small cell lung cancer | 0.0 |
| NCI-H526- Small cell lung cancer | 0.0 |
| NCI-N417- Small cell lung cancer | 0.0 |
| NCI-H82- Small cell lung cancer | 0.1 |
| NCI-H157- Squamous cell lung cancer (metastasis) | 42.0 |
| NCI-H1155- Large cell lung cancer | 0.5 |
| NCI-H1299- Large cell lung cancer | 23.5 |
| NCI-H727- Lung carcinoid | 0.3 |
| NCI-UMC-11- Lung carcinoid | 0.1 |
| LX-1- Small cell lung cancer | 0.0 |
| Colo-205- Colon cancer | 0.0 |
| KM12- Colon cancer | 0.9 |
| KM20L2- Colon cancer | 0.2 |
| NCI-H716- Colon cancer | 1.0 |
| SW-48- Colon adenocarcinoma | 0.0 |
| SW1116- Colon adenocarcinoma | 0.5 |
| LS 174T- Colon adenocarcinoma | 0.8 |
| SW-948- Colon adenocarcinoma | 0.0 |
| SW-480- Colon adenocarcinoma | 0.0 |
| NCI-SNU-5- Gastric carcinoma | 3.5 |
| KATO III- Gastric carcinoma | 0.7 |
| NCI-SNU-16- Gastric carcinoma | 6.3 |
| NCI-SNU-1- Gastric carcinoma | 0.4 |
| KF-1- Gastric adenocarcinoma | 0.0 |
| RF-48- Gastric adenocarcinoma | 0.0 |
| MKN-45- Gastric carcinoma | 1.6 |
| NCI-N87- Gastric carcinoma | 1.0 |
| OVCAR-5- Ovarian carcinoma | 0.9 |
| RL95-2- Uterine carcinoma | 1.4 |
| HelaS3- Cervical adenocarcinoma | 1.6 |
| Ca Ski- Cervical epidermoid carcinoma (metastasis) | 52.9 |
| ES-2- Ovarian clear cell carcinoma | 26.8 |
| Ramos- Stimulated with PMA/ionomycin 6 h | 0.0 |
| Ramos- Stimulated with PMA/ionomycin 14 h | 0.1 |
| MEG-01- Chronic myelogenous leukemia (megokaryoblast) | 0.9 |
| Raji- Burkitt's lymphoma | 0.1 |
| Daudi- Burkitt's lymphoma | 0.2 |
| U266- B-cell plasmacytoma | 0.0 |
| CA46- Burkitt's lymphoma | 0.0 |
| RL- non-Hodgkin's B-cell lymphoma | 0.0 |
| JM1- pre-B-cell lymphoma | 0.0 |
| Jurkat- T cell leukemia | 0.0 |
| TF-1- Erythroleukemia | 0.3 |
| HUT 78- T-cell lymphoma | 0.0 |
| U937- Histiocytic lymphoma | 0.0 |
| KU-812- Myelogenous leukemia | 0.2 |
| 769-P- Clear cell renal carcinoma | 17.7 |
| Caki-2- Clear cell renal carcinoma | 1.7 |
| SW 839- Clear cell renal carcinoma | 71.7 |
| Rhabdoid kidney tumor | 3.0 |
| Hs766T- Pancreatic carcinoma (LN metastasis) | 15.4 |
| CAPAN-1- Pancreatic adenocarcinoma (liver metastasis) | 6.9 |
| SU86.86- Pancreatic carcinoma (liver metastasis) | 14.6 |
| BxPC-3- Pancreatic adenocarcinoma | 3.6 |
| HPAC- Pancreatic adenocarcinoma | 4.0 |
| MIA PaCa-2- Pancreatic carcinoma | 3.4 |
| CFPAC-1- Pancreatic ductal adenocarcinoma | 28.3 |
| PANC-1- Pancreatic epithelioid ductal carcinoma | 16.2 |
| T24- Bladder carcinma (transitional cell) | 12.1 |
| 5637- Bladder carcinoma | 3.1 |
| HT-1197- Bladder carcinoma | 14.6 |
| UM-UC-3- Bladder carcinma (transitional cell) | 4.6 |
| A204- Rhabdomyosarcoma | 0.7 |
| HT-1080- Fibrosarcoma | 15.8 |
| MG-63- Osteosarcoma | 53.2 |
| SK-LMS-1- Leiomyosarcoma (vulva) | 55.5 |
| SJRH30- Rhabdomyosarcoma (met to bone marrow) | 1.7 |
| A431- Epidermoid carcinoma | 1.6 |
| WM266-4- Melanoma | 0.5 |
| DU 145- Prostate carcinoma (brain metastasis) | 1.8 |
| MDA-MB-468- Breast adenocarcinoma | 1.8 |
| SCC-4- Squamous cell carcinoma of tongue | 1.3 |
| SCC-9- Squamous cell carcinoma of tongue | 1.0 |
| SCC-15- Squamous cell carcinoma of tongue | 1.3 |
| CAL 27- Squamous cell carcinoma of tongue | 20.7 |

Column A - Rel. Exp. (%) Ag1148, Run 163476715

TABLE KG
Panel 4D

| Tissue Name | A |
|---|---|
| Secondary Th1 act | 0.0 |
| Secondary Th2 act | 0.0 |
| Secondary Tr1 act | 0.0 |
| Secondary Th1 rest | 0.0 |
| Secondary Th2 rest | 0.0 |
| Secondary Tr1 rest | 0.0 |
| Primary Th1 act | 0.1 |
| Primary Th2 act | 0.3 |
| Primary Tr1 act | 0.5 |
| Primary Th1 rest | 0.5 |
| Primary Th2 rest | 0.4 |
| Primary Tr1 rest | 0.1 |
| CD45RA CD4 lymphocyte act | 39.0 |
| CD45RO CD4 lymphocyte act | 0.1 |
| CD8 lymphocyte act | 0.1 |
| Secondary CD8 lymphocyte rest | 0.0 |
| Secondary CD8 lymphocyte act | 0.0 |
| CD4 lymphocyte none | 0.0 |
| 2ry Th1/Th2/Tr1_anti-CD95 CH11 | 0.3 |
| LAK cells rest | 0.0 |
| LAK cells IL-2 | 0.1 |
| LAK cells IL-2 + IL-12 | 0.3 |
| LAK cells IL-2 + IFN gamma | 0.4 |
| LAK cells IL-2 + IL-18 | 0.0 |
| LAK cells PMA/ionomycin | 0.3 |
| NK Cells IL-2 rest | 0.0 |
| Two Way MLR 3 day | 0.1 |
| Two Way MLR 5 day | 0.2 |
| Two Way MLR 7 day | 0.0 |
| PBMC rest | 0.0 |
| PBMC PWM | 0.4 |
| PBMC PHA-L | 0.3 |
| Ramos (B cell) none | 0.7 |
| Ramos (B cell) ionomycin | 1.4 |
| B lymphocytes PWM | 1.0 |
| B lymphocytes CD40L and IL-4 | 0.6 |
| EOL-1 dbcAMP | 0.3 |
| EOL-1 dbcAMP PMA/ionomycin | 0.4 |
| Dendritic cells none | 0.0 |
| Dendritic cells LPS | 0.0 |
| Dendritic cells anti-CD40 | 0.1 |
| Monocytes rest | 0.1 |
| Monocytes LPS | 0.1 |
| Macrophages rest | 0.2 |
| Macrophages LPS | 0.0 |
| HUVEC none | 49.3 |
| HUVEC starved | 41.2 |
| HUVEC IL-1beta | 17.7 |
| HUVEC IFN gamma | 40.3 |
| HUVEC TNF alpha + IFN gamma | 30.1 |
| HUVEC TNF alpha + IL4 | 42.6 |
| HUVEC IL-11 | 19.8 |
| Lung Microvascular EC none | 63.7 |
| Lung Microvascular EC TNFalpha + IL-1beta | 46.7 |
| Microvascular Dermal EC none | 93.3 |
| Microsvasular Dermal EC TNFalpha + IL-1beta | 71.7 |
| Bronchial epithelium TNFalpha + IL1beta | 16.7 |
| Small airway epithelium none | 5.3 |
| Small airway epithelium TNFalpha + IL-1beta | 29.5 |
| Coronery artery SMC rest | 46.0 |
| Coronery artery SMC TNFalpha + IL-1beta | 48.0 |
| Astrocytes rest | 13.0 |
| Astrocytes TNFalpha + IL-1beta | 28.7 |
| KU-812 (Basophil) rest | 0.2 |
| KU-812 (Basophil) PMA/ionomycin | 0.7 |
| CCD1106 (Keratinocytes) none | 4.5 |
| CCD1106 (Keratinocytes) TNFalpha + IL-1beta | 12.7 |
| Liver cirrhosis | 7.1 |
| Lupus kidney | 2.5 |
| NCI-H292 none | 23.7 |
| NCI-H292 IL-4 | 12.3 |
| NCI-H292 IL-9 | 11.7 |
| NCI-H292 IL-13 | 12.2 |
| NCI-H292 IFN gamma | 13.5 |
| HPAEC none | 42.3 |
| HPAEC TNF alpha + IL-1 beta | 51.8 |
| Lung fibroblast none | 24.8 |
| Lung fibroblast TNF alpha + IL-1 beta | 6.7 |
| Lung fibroblast IL-4 | 41.8 |
| Lung fibroblast IL-9 | 32.8 |
| Lung fibroblast IL-13 | 89.5 |
| Lung fibroblast IFN gamma | 69.3 |
| Dermal fibroblast CCD1070 rest | 100.0 |
| Dermal fibroblast CCD1070 TNF alpha | 77.9 |
| Dermal fibroblast CCD1070 IL-1 beta | 95.3 |
| Dermal fibroblast IFN gamma | 5.8 |
| Dermal fibroblast IL-4 | 8.0 |
| IBD Colitis 2 | 3.0 |
| IBD Crohn's | 5.3 |
| Colon | 2.5 |
| Lung | 11.4 |
| Thymus | 9.1 |
| Kidney | 2.6 |

Column A - Rel. Exp. (%) Ag1148, Run 145386435

HASS Panel v1.0 Summary: Ag1148 Expression of the CG55688-01 gene was highest in T24 cells (CT=27.9). This gene was also expressed at significant level in CaPaN and U87 cancer cell lines, as well as in primary astrocytes, renal epithelial cells and melanocytes in culture. Gene expression was induced by a combination of low oxygen tension and acidic pH in U8 cell lines, suggesting a regulation in vivo may also occur in regions of low pH and low oxygen. Therapeutic modulation of the activity of this gene or its protein product is useful in the treatment of cancer.
PGI1.0 Summary: Ag1148 Highest expression of this gene was detected in emphysema lung (CT=25.2). High expression of this gene was also detected in lung fibrosis, asthma, emphysema and ulcerative colitis samples. Therapeutic modulation of the activity of this gene or its protein product using nucleic acid, protein, antibody or small molecule drugs is useful in the treatment of lung fibrosis, asthma, emphysema and ulcerative colitis.
Panel 1.3D Summary: Ag1148 The expression of this gene was highest in a sample derived from a melanoma cell line (Hs.688(A).T) (CT=27). In addition, there is significant expression in a related melanoma cell line (Hs.688(B).T) as well as a cluster of brain cancer cell lines and renal cancer cell lines. T Therapeutic modulation of the activity of this gene or its protein product is useful in the treatment of melanoma, renal cancer or brain cancer.

This panel shows significant expression of this gene in metabolic tissues, including adipose, pancreas, adrenal, thyroid, pituitary, skeletal muscle and adult and fetal liver. The CG55688-01 gene encodes CYR61, which belongs to the insulin-like growth factor binding protein family and may play myriad roles in metabolic regulation. Therapeutic modulation of the activity of this gene or its protein product using nucleic acid, protein, antibody or small molecule drugs is useful for the treatment of metabolic and endocrine diseases, including obesity and Types 1 and 2 diabetes.

In addition, this gene was expressed at low levels in several brain regions including hippocampus, cortex, substantia nigra, thalamus, amygdala, and the fetal brain. Cry61 is an immediate early gene that has been implicated in memory formation and synaptic plasticity (Albrecht C, von Der Kammer H, Mayhaus M, Klaudiny J, Schweizer M, Nitsch R M. Muscarinic acetylcholine receptors induce the expression of the immediate early growth regulatory gene CYR61. J Biol Chem Sep. 15, 2000;275(37):28929-36). It has also been shown to be upregulated during the development of the hippocampus, which is a critical brain region for the formation of long-term memory (Chung K C, Ahn Y S. Expression of immediate early gene cyr61 during the differentiation of immortalized embryonic hippocampal neuronal cells. Neurosci Lett Oct. 23, 1998;255(3): 155-8). Therefore, this gene, expressed protein, antibodies or small molecule drug targeting this gene or gene product is useful in the treatment of dementia (Alzheimer's, vascular, etc) or for memory enhancement.

Panel 2D Summary: Ag1148 Highest expression of this gene was found in normal bladder tissue and a kidney cancer sample (CTs=28). In addition, there was significant expression of this gene associated with ovarian and prostate derived tissues and a number of kidney samples. Therapeutic modulation of the activity of this gene or its protein product is useful in the treatment of kidney cancer, ovarian cancer or prostate cancer.

Panel 3D Summary: Ag1148 Highest expression of this gene was detected in lung cancer cell line NCI-H292 (CT=28). Significant expression of this gene was also seen in a number of cancer cell lines derived from brain, renal, pancreatic, bladder cancers and sarcomas. Therapeutic modulation of the activity of this gene or its protein product is useful in the treatment of these cancers.

Panel 4D Summary: Ag1148 This gene, a Cyr61 homolog, was expressed at moderate levels (CTs=28-32) in resting and cytokine-stimulated HUVEC, lung microvascular endothelial cells, coronary artery smooth muscle cells, bronchial epithelial cells, small airway epithelial cells, astrocytes, pulmonary artery endothelial cells, lung fibroblasts, and dermal fibroblasts. Based upon this expression pattern and a role for Cyr61 in vascular biology (Babic A M, Kireeva M L, Kolesnikova T V, Lau L F CYR61, a product of a growth factor-inducible immediate early gene, promotes angiogenesis and tumor growth. Proc Natl Acad Sci USA May 26, 1998;95(11):6355-60), therapeutic modulation of the acitivity of this gene or its protein product is useful in the treatment of inflammatory or autoimmune diseases, including Crohn's disease, ulcerative colitis, multiple sclerosis, chronic obstructive pulmonary disease, asthma, emphysema, rheumatoid arthritis, or psoriasis.

L. CG56768-01: Wnt-5A

Expression of gene CG56768-01 was assessed using the primer-probe set Ag1450, described in Table LA. Results of the RTQ-PCR runs are shown in Tables LB, LC, LD and LE.

TABLE LA
Probe Name Ag1450

| | | | Start | SEQ ID |
|---|---|---|---|---|
| Primers | Sequences | Length | Position | No |
| Forward | 5′-ccaagttcttcctagtgg | 22 | 82 | 322 |
| | cttt-3′ | | | |
| Probe | TET-5′-tttctccttcgccc | 26 | 113 | 323 |
| | aggttgtaattg-3′-TAMRA | | | |
| Reverse | 5′-atacctagcgaccaccaa | 22 | 145 | 324 |
| | gaat-3′ | | | |

TABLE LB
Ardais Panel 1.1

| Tissue Name | A |
|---|---|
| Lung adenocarcinoma SI A | 9.0 |
| Lung adenocarcinoma SI B | 3.0 |
| Lung adenocarcinoma SI B NAT | 2.5 |
| Lung adenocarcinoma SI C | 0.6 |
| Lung adenocarcinoma SI C NAT | 2.3 |
| Lung adenocarcinoma SII A | 1.1 |
| Lung adenocarcinoma SII A NAT | 2.2 |
| Lung adenocarcinoma SII C NAT | 5.9 |
| Lung adenocarcinoma SIII A | 5.5 |
| Lung adenocarcinoma SIII B | 2.8 |
| Lung adenocarcinoma SIII C | 7.7 |
| Lung SCC SI A | 1.8 |
| Lung SCC SI B NAT | 3.2 |
| Lung SCC SI C | 1.1 |
| Lung SCC SI C NAT | 11.8 |
| Lung SCC SI D | 100.0 |
| Lung SCC SI D NAT | 0.5 |
| Lung SCC SII A | 3.8 |
| Lung SCC SII B | 1.3 |
| Lung SCC SIII A | 3.0 |
| Lung SCC SIII A NAT | 1.3 |

Column A Rel. Exp. (%) Agl450, Run 306913817

TABLE LC
Panel 1.2
Column A - Rel. Exp. (%) Ag1450, Run 140179432
Column B - Rel. Exp. (%) Ag1450, Run 140448122

| Tissue Name | A | B |
|---|---|---|
| Endothelial cells | 0.5 | 0.5 |
| Heart (Fetal) | 1.5 | 2.1 |
| Pancreas | 0.2 | 0.6 |
| Pancreatic ca. CAPAN 2 | 0.0 | 0.0 |
| Adrenal Gland | 0.9 | 1.2 |
| Thyroid | 0.3 | 0.3 |
| Salivary gland | 21.6 | 20.0 |
| Pituitary gland | 2.3 | 0.2 |
| Brain (fetal) | 0.2 | 0.1 |
| Brain (whole) | 0.1 | 0.1 |
| Brain (amygdala) | 0.4 | 0.5 |
| Brain (cerebellum) | 0.6 | 0.4 |
| Brain (hippocampus) | 1.2 | 1.4 |
| Brain (thalamus) | 0.6 | 0.6 |
| Cerebral Cortex | 3.3 | 4.6 |
| Spinal cord | 0.2 | 0.1 |
| glio/astro U87-MG | 51.8 | 64.6 |
| glio/astro U-118-MG | 46.3 | 55.5 |
| astrocytoma SW1783 | 13.4 | 14.1 |
| neuro*; met SK-N-AS | 2.1 | 2.0 |
| astrocytoma SF-539 | 3.1 | 2.5 |
| astrocytoma SNB-75 | 12.0 | 10.2 |
| glioma SNB-19 | 0.4 | 1.1 |
| glioma U251 | 2.9 | 4.0 |
| glioma SF-295 | 100.0 | 100.0 |
| Heart | 4.1 | 5.6 |
| Skeletal Muscle | 5.6 | 7.7 |
| Bone marrow | 0.1 | 0.1 |
| Thymus | 0.2 | 0.1 |
| Spleen | 0.4 | 0.6 |
| Lymph node | 0.1 | 0.1 |
| Colorectal Tissue | 0.8 | 1.7 |
| Stomach | 0.2 | 0.6 |
| Small intestine | 3.4 | 3.4 |
| Colon ca. SW480 | 10.2 | 18.3 |
| Colon ca.* SW620 (SW480 met) | 0.0 | 0.0 |
| Colon ca. HT29 | 0.0 | 0.0 |
| Colon ca. HCT-116 | 0.0 | 0.0 |
| Colon ca. CaCo-2 | 1.1 | 1.2 |
| Colon ca. Tissue (ODO3866) | 1.2 | 1.3 |
| Colon ca. HCC-2998 | 0.8 | 0.9 |
| Gastric ca.* (liver met) NCI-N87 | 0.0 | 0.1 |
| Bladder | 4.6 | 8.1 |
| Trachea | 0.9 | 0.1 |
| Kidney | 5.8 | 5.6 |
| Kidney (fetal) | 5.1 | 4.6 |
| Renal ca. 786-0 | 1.2 | 1.5 |
| Renal ca. A498 | 0.5 | 0.9 |
| Renal ca. RXF 393 | 0.8 | 1.4 |
| Renal ca. ACHN | 0.9 | 1.2 |
| Renal ca. UO-31 | 6.6 | 10.4 |
| Renal ca. TK-10 | 0.2 | 0.2 |
| Liver | 0.7 | 0.9 |
| Liver (fetal) | 0.3 | 0.4 |
| Liver ca. (hepatoblast) HepG2 | 0.0 | 0.0 |
| Lung | 0.1 | 0.5 |
| Lung (fetal) | 0.2 | 0.3 |
| Lung ca. (small cell) LX-1 | 0.0 | 0.0 |
| Lung ca. (small cell) NCI-H69 | 0.2 | 0.3 |
| Lung ca. (s. cell var.) SHP-77 | 0.1 | 0.1 |
| Lung ca. (large cell)NCI-H460 | 20.0 | 27.7 |
| Lung ca. (non-sm. cell) A549 | 0.1 | 0.1 |
| Lung ca. (non-s. cell) NCI-H23 | 0.2 | 0.3 |
| Lung ca. (non-s. cell) HOP-62 | 70.7 | 58.2 |
| Lung ca. (non-s. cl) NCI-H522 | 1.7 | 2.4 |
| Lung ca. (squam.) SW 900 | 12.8 | 22.5 |
| Lung ca. (squam.) NCI-H596 | 0.6 | 1.0 |
| Mammary gland | 1.1 | 1.3 |
| Breast ca.* (pl. ef) MCF-7 | 0.2 | 0.2 |
| Breast ca.* (pl. ef) MDA-MB-231 | 0.0 | 0.0 |
| Breast ca.* (pl. ef) T47D | 0.0 | 0.0 |
| Breast ca. BT-549 | 1.3 | 1.3 |
| Breast ca. MDA-N | 0.0 | 0.0 |
| Ovary | 10.7 | 19.3 |
| Ovarian ca. OVCAR-3 | 18.0 | 16.3 |
| Ovarian ca. OVCAR-4 | 14.5 | 16.5 |
| Ovarian ca. OVCAR-5 | 1.4 | 1.5 |
| Ovarian ca. OVCAR-8 | 2.5 | 2.6 |
| Ovarian ca. IGROV-1 | 15.6 | 9.4 |
| Ovarian ca. (ascites) SK-OV-3 | 1.3 | 2.4 |
| Uterus | 3.5 | 2.5 |
| Placenta | 8.7 | 1.2 |
| Prostate | 2.0 | 2.9 |
| Prostate ca.* (bone met) PC-3 | 17.2 | 17.4 |
| Testis | 0.2 | 0.4 |
| Melanoma Hs688(A).T | 17.2 | 22.2 |
| Melanoma* (met) Hs688(B).T | 16.2 | 18.7 |
| Melanoma UACC-62 | 2.2 | 2.7 |
| Melanoma M14 | 0.8 | 1.0 |
| Melanoma LOX IMVI | 4.7 | 5.6 |
| Melanoma* (met) SK-MEL-5 | 0.5 | 0.8 |

TABLE LD
Panel 2D
Colmn A - Rel. Exp. (%) Ag1450, Run 145090529
Column B - Rel. Exp. (%) Ag1450, Run 148500417

| Tissue Name | A | B |
|---|---|---|
| Normal Colon | 13.7 | 10.8 |
| CC Well to Mod Diff (ODO3866) | 20.7 | 15.8 |
| CC Margin (ODO3866) | 9.8 | 5.1 |
| CC Gr. 2 rectosigmoid (ODO3868) | 14.2 | 8.4 |
| CC Margin (ODO3868) | 3.6 | 1.6 |
| CC Mod Diff (ODO3920) | 12.2 | 10.7 |
| CC Margin (ODO3920) | 1.9 | 2.2 |
| CC Gr. 2 ascend colon (ODO3921) | 29.9 | 33.7 |
| CC Margin (ODO3921) | 9.1 | 6.7 |
| CC from Partial Hepatectomy | 5.3 | 4.9 |
| (ODO4309) Mets | | |
| Liver Margin (ODO4309) | 4.5 | 4.7 |
| Colon mets to lung (OD04451-01) | 9.3 | 4.4 |
| Lung Margin (OD04451-02) | 12.3 | 5.9 |
| Normal Prostate 6546-1 | 15.4 | 5.7 |
| Prostate Cancer (OD04410) | 21.0 | 8.7 |
| Prostate Margin (OD04410) | 42.6 | 33.4 |
| Prostate Cancer (OD04720-01) | 23.5 | 22.2 |
| Prostate Margin (OD04720-02) | 32.1 | 25.2 |
| Normal Lung 061010 | 15.7 | 12.0 |
| Lung Met to Muscle (ODO4286) | 1.1 | 0.8 |
| Muscle Margin (ODO4286) | 1.6 | 0.3 |
| Lung Malignant Cancer (OD03126) | 40.9 | 20.7 |
| Lung Margin (OD03126) | 20.3 | 15.9 |
| Lung Cancer (OD04404) | 100.0 | 100.0 |
| Lung Margin (OD04404) | 27.2 | 25.9 |
| Lung Cancer (OD04565) | 31.4 | 29.3 |
| Lung Margin (OD04565) | 6.8 | 5.1 |
| Lung Cancer (OD04237-01) | 7.1 | 4.3 |
| Lung Margin (OD04237-02) | 11.3 | 7.0 |
| Ocular Mel Met to Liver (ODO4310) | 0.0 | 0.0 |
| Liver Margin (ODO4310) | 4.1 | 1.5 |
| Melanoma Mets to Lung (OD04321) | 7.6 | 4.8 |
| Lung Margin (OD04321) | 33.9 | 20.4 |
| Normal Kidney | 19.9 | 9.2 |
| Kidney Ca, Nuclear grade 2 | 32.5 | 25.3 |
| (OD04338) | | |
| Kidney Margin (OD04338) | 9.8 | 7.9 |
| Kidney Ca Nuclear grade ½ | 29.5 | 23.7 |
| (OD04339) | | |
| Kidney Margin (OD04339) | 3.6 | 2.3 |
| Kidney Ca, Clear cell type (OD04340) | 4.0 | 3.7 |
| Kidney Margin (OD04340) | 14.8 | 9.3 |
| Kidney Ca, Nuclear grade 3 | 4.5 | 3.0 |
| (OD04348) | | |
| Kidney Margin (OD04348) | 8.0 | 4.5 |
| Kidney Cancer (OD04622-01) | 4.5 | 4.1 |
| Kidney Margin (OD04622-03) | 11.8 | 4.2 |
| Kidney Cancer (OD04450-01) | 26.4 | 12.9 |
| Kidney Margin (OD04450-03) | 13.7 | 5.0 |
| Kidney Cancer 8120607 | 1.7 | 1.0 |
| Kidney Margin 8120608 | 3.8 | 1.5 |
| Kidney Cancer 8120613 | 0.4 | 0.4 |
| Kidney Margin 8120614 | 8.0 | 5.2 |
| Kidney Cancer 9010320 | 10.7 | 7.0 |
| Kidney Margin 9010321 | 11.6 | 5.4 |
| Normal Uterus | 11.2 | 5.9 |
| Uterus Cancer 064011 | 59.9 | 37.4 |
| Normal Thyroid | 16.3 | 5.7 |
| Thyroid Cancer 064010 | 33.0 | 17.0 |
| Thyroid Cancer A302152 | 14.4 | 9.2 |
| Thyroid Margin A302153 | 11.9 | 7.9 |
| Normal Breast | 20.3 | 10.8 |
| Breast Cancer (OD04566) | 10.7 | 5.8 |
| Breast Cancer (OD04590-01) | 10.4 | 7.1 |
| Breast Cancer Mets | 7.6 | 3.0 |
| (OD04590-03) | | |
| Breast Cancer Metastasis | 7.4 | 5.4 |
| (OD04655-05) | | |
| Breast Cancer 064006 | 13.9 | 9.4 |
| Breast Cancer 1024 | 40.9 | 25.9 |
| Breast Cancer 9100266 | 9.8 | 5.4 |
| Breast Margin 9100265 | 13.8 | 10.7 |
| Breast Cancer A209073 | 45.7 | 33.0 |
| Breast Margin A209073 | 11.0 | 5.6 |
| Normal Liver | 4.2 | 2.6 |
| Liver Cancer 064003 | 0.6 | 0.3 |
| Liver Cancer 1025 | 2.9 | 2.5 |
| Liver Cancer 1026 | 7.3 | 6.3 |
| Liver Cancer 6004-T | 7.0 | 2.2 |
| Liver Tissue 6004-N | 0.7 | 0.7 |
| Liver Cancer 6005-T | 11.8 | 5.7 |
| Liver Tissue 6005-N | 0.6 | 0.7 |
| Normal Bladder | 12.1 | 11.7 |
| Bladder Cancer 1023 | 4.9 | 2.9 |
| Bladder Cancer A302173 | 59.5 | 27.4 |
| Bladder Cancer (OD04718-01) | 12.8 | 11.7 |
| Bladder Normal Adjacent | 2.8 | 0.7 |
| (OD04718-03) | | |
| Normal Ovary | 29.7 | 19.8 |
| Ovarian Cancer 064008 | 25.2 | 30.8 |
| Ovarian Cancer (OD04768-07) | 2.8 | 2.5 |
| Ovary Margin (OD04768-08) | 4.0 | 2.6 |
| Normal Stomach | 6.7 | 5.4 |
| Gastric Cancer 9060358 | 4.9 | 1.6 |
| Stomach Margin 9060359 | 7.1 | 5.0 |
| Gastric Cancer 9060395 | 30.1 | 25.5 |
| Stomach Margin 9060394 | 1.5 | 5.6 |
| Gastric Cancer 9060397 | 18.3 | 21.9 |
| Stomach Margin 9060396 | 3.7 | 1.8 |
| Gastric Cancer 064005 | 16.3 | 18.0 |

TABLE LE
Panel 4.1D

| Tissue Name | A |
|---|---|
| Secondary Th1 act | 0.3 |
| Secondary Th2 act | 0.3 |
| Secondary Tr1 act | 0.3 |
| Secondary Th1 rest | 0.3 |
| Secondary Th2 rest | 0.0 |
| Secondary Tr1 rest | 0.1 |
| Primary Th1 act | 0.0 |
| Primary Th2 act | 0.0 |
| Primary Tr1 act | 0.0 |
| Primary Th1 rest | 0.0 |
| Primary Th2 rest | 0.0 |
| Primary Tr1 rest | 0.0 |
| CD45RA CD4 lymphocyte act | 44.4 |
| CD45RO CD4 lymphocyte act | 0.0 |
| CD8 lymphocyte act | 0.0 |
| Secondary CD8 lymphocyte rest | 0.1 |
| Secondary CD8 lymphocyte act | 0.0 |
| CD4 lymphocyte none | 0.0 |
| 2ry Th1/Th2/Tr1_anti-CD95 CH11 | 0.0 |
| LAK cells rest | 0.2 |
| LAK cells IL-2 | 0.0 |
| LAK cells IL-2 + IL-12 | 0.1 |
| LAK cells IL-2 + IFN gamma | 0.0 |
| LAK cells IL-2 + IL-18 | 0.1 |
| LAK cells PMA/ionomycin | 0.1 |
| NK Cells IL-2 rest | 0.1 |
| Two Way MLR 3 day | 0.2 |
| Two Way MLR 5 day | 1.2 |
| Two Way MLR 7 day | 0.1 |
| PBMC rest | 0.0 |
| PBMC PWM | 2.9 |
| PBMC PHA-L | 1.5 |
| Ramos (B cell) none | 0.0 |
| Ramos (B cell) ionomycin | 0.0 |
| B lymphocytes PWM | 0.1 |
| B lymphocytes CD40L and IL-4 | 0.4 |
| EOL-1 dbcAMP | 0.0 |
| EOL-1 dbcAMP PMA/ionomycin | 0.0 |
| Dendritic cells none | 2.5 |
| Dendritic cells LPS | 20.6 |
| Dendritic cells anti-CD40 | 10.7 |
| Monocytes rest | 0.0 |
| Monocytes LPS | 20.4 |
| Macrophages rest | 0.2 |
| Macrophages LPS | 5.9 |
| HUVEC none | 0.0 |
| HUVEC starved | 0.0 |
| HUVEC IL-1beta | 0.1 |
| HUVEC IFN gamma | 0.2 |
| HUVEC TNF alpha + IFN gamma | 0.7 |
| HUVEC TNF alpha + IL4 | 0.5 |
| HUVEC IL-11 | 0.4 |
| Lung Microvascular EC none | 0.2 |
| Lung Microvascular EC TNFalpha + IL-1beta | 0.4 |
| Microvascular Dermal EC none | 0.0 |
| Microsvasular Dermal EC TNFalpha + IL-1beta | 0.1 |
| Bronchial epithelium TNFalpha + IL1beta | 3.7 |
| Small airway epithelium none | 5.0 |
| Small airway epithelium TNFalpha + IL-1beta | 0.4 |
| Coronery artery SMC rest | 2.4 |
| Coronery artery SMC TNFalpha + IL-1beta | 3.3 |
| Astrocytes rest | 2.8 |
| Astrocytes TNFalpha + IL-1beta | 10.8 |
| KU-812 (Basophil) rest | 0.3 |
| KU-812 (Basophil) PMA/ionomycin | 0.5 |
| CCD1106 (Keratinocytes) none | 3.0 |
| CCD1106 (Keratinocytes) TNFalpha + IL-1beta | 1.3 |
| Liver cirrhosis | 0.4 |
| NCI-H292 none | 0.0 |
| NCI-H292 IL-4 | 0.1 |
| NCI-H292 IL-9 | 0.3 |
| NCI-H292 IL-13 | 2.3 |
| NCI-H292 IFN gamma | 0.2 |
| HPAEC none | 0.0 |
| HPAEC TNF alpha + IL-1 beta | 0.5 |
| Lung fibroblast none | 38.2 |
| Lung fibroblast TNF alpha + IL-1 beta | 75.3 |
| Lung fibroblast IL-4 | 39.0 |
| Lung fibroblast IL-9 | 71.7 |
| Lung fibroblast IL-13 | 25.0 |
| Lung fibroblast IFN gamma | 41.2 |
| Dermal fibroblast CCD1070 rest | 84.1 |
| Dermal fibroblast CCD1070 TNF alpha | 76.8 |
| Dermal fibroblast CCD1070 IL-1 beta | 100.0 |
| Dermal fibroblast IFN gamma | 8.9 |
| Dermal fibroblast IL-4 | 10.6 |
| Dermal Fibroblasts rest | 6.1 |
| Neutrophils TNFa + LPS | 1.1 |
| Neutrophils rest | 1.5 |
| Colon | 0.9 |
| Lung | 3.6 |
| Thymus | 1.7 |
| Kidney | 2.6 |

Column A - Rel. Exp. (%) Ag1450, Run 181080827

Ardais Panel 1.1 Summary: Ag1450 Highest expression of the CG56768-01 gene was seen in a lung cancer sample (CT=21). Expression of this gene was higher in this cancer sample relative to the normal adjacent tissue sample (CT=29). Gene or protein expression levels are useful for the detection of lung cancer. Therapeutic modulation of the activity of this gene or its protein product using nucleic acid, protein, antibody or small molecule drugs is useful in the treatment of lung cancer.
Panel 1.2 Summary: Ag1450 Highest expression of this gene was detected in glioma cell line SF-295 (CT=22). Ibis gene was overexpressed in cell lines derived from CNS malignancies when compared to the low to moderate expression in the samples derived from normal CNS tissue. In addition, there was consistently high expression of this gene in melanoma cell lines, ovarian cancer cell lines and lung cancer cell lines. The CG56768-01 gene encodes a putative Wnt5a-like protein. The Wnt genes belong to a family of protooncogenes with at least 13 known members that are expressed in species ranging from Drosophila to man. The name Wnt denotes the relationship of this family to the Drosophila segment polarity gene ‘wingless’ and to its vertebrate ortholog, Int1, a mouse protooncogene (OMIM 164975, 164820). Therapeutic modulation of the activity of this gene or its protein product using nucleic acid, protein, antibody or small molecule drugs is useful for the treatment of brain cancer, melanoma, ovarian cancer and/or lung cancer.

Significant levels of expression of this gene were detected in all the regions of the brain examined including amygdala, hippocampus, substantia nigra, thalamus, cerebellum, cerebral cortex, and spinal cord. Wnt-5A signalling is believed to play a critical role in cadherin-mediated cell organization. Cadherins can act as axon guidance and cell adhesion proteins, specifically during development and in the response to injury. Therapeutic modulation of the activity of this gene or its protein product is useful in inducing a compensatory synaptogenic response to neuronal death in Alzheimer's disease, Parkinson's disease, Huntington's disease, spinocerebellar ataxia, progressive supranuclear palsy, ALS, head trauma, stroke, or any other disease/condition associated with neuronal loss.

Among tissues with metabolic or endocrine function, this gene was expressed at moderate levels in pancreas, adrenal gland, thyroid, pituitary and liver. In addition, this gene was expressed at high levels in skeletal muscle (CT=27). These observations suggest that the Wnt-5A-like protein encoded by this gene may be secreted from skeletal muscle as a paracrine or endocrine signalling molecule acting on other insulin-responsive tissues (i.e., adipose and pancreatic beta cells). Therapeutic modulation of the activity of this gene or its protein product using nucleic acid, protein, antibody or small molecule drugs is useful in the treatment of metabolic diseases involving skeletal muscle, including Type 2 diabetes.

Panel 2D Summary: Ag1450 Highest expression of this gene was detected in a lung cancer sample (CTs=28-29). This gene was overexpressed in number of cancer tissues relative to the adjacent normal colon, lung, kidney, breast, and stomach. These results are consistent with the observation that the Wnt-5A gene appears to be up-regulated in a number of human malignancies (lozzo R. V., Eichstetter I., Danielson K. G., 1995, Aberrant expression of the growth factor Wnt-5A in human malignancy. Cancer Res. 55: 3495-3499). Therapeutic modulation of the activity of this gene or its protein product is of use in the treatment of colon, lung, kidney, breast or gastric cancers.

Panel 4.1D Summary: Ag1450 Highest expression of this gene was detected in IL-1 beta activated dermal fibroblasts (CT=26). This gene was expressed mainly in fibroblasts and in LPS-activated monocytes, macrophages and dendritic cells. WNTs are secreted signalling molecules that regulate cell fate and behavior and are involved in embryonic development and hematopoiesis. During inflammation, the Wnt5a-like protein encoded by this gene could potentiate the inflammatory response by acting as an autocrine factor and stimulating monocyte differentiation into dendritic cells as well as by allowing dendritic cells to mature into potent antigen presenting cells. Alternatively, this gene may influence the differentiation of other cell types in the microenvironment including synovial tissues (Sen M., Lauterbach K., El-Gabalawy H., Firestein G. S., Corr M., Carson D. A., 2000, Expression and function of wingless and frizzled homologs in rheumatoid arthritis. Proc. Natl. Acad. Sci. USA 97: 2791-2796). Therapeutic modulation of the activity of this gene or its protein product using nucleic acid, protein, antibody or small molecule drugs is important in reducing or blocking inflammation associated with rheumatoid arthritis, asthma, allergy, psoriasis, IBD and Crohn's disease.

M. CG59253-01 and CG59253-02: Semaphorin Precursor

Expression of gene CG59253-01 and CG59253-02 was assessed using the primer-probe sets Ag1492 and Ag2441, described in Tables MA and MB. Results of the RTQ-PCR runs are shown in Tables MC, MD, ME and MF. CG59253-01 represents a full-length physical clone.

TABLE MA
Probe Name Ag1492

| | | | Start | SEQ ID |
|---|---|---|---|---|
| Primers | Sequences | Length | Position No | |
| Forward | 5′-ctgaagctggcatggtac | 22 | 501 | 325 |
| | ttaa-3′ | | | |
| Probe | TET-5′-cagtcctttctctt | 26 | 460 | 326 |
| | tgaacgacagcg-3′-TAMRA | | | |
| Reverse | 5′-ttgtaggcttcaatctct | 22 | 432 | 327 |
| | tcca-3′ | | | |

TABLE MB
Probe Name Ag2441

| | | | Start | SEQ ID |
|---|---|---|---|---|
| Primers | Sequences | Length | Position | No |
| Forward | 5′-tgctatgaaaggcaagca | 21 | 1506 | 328 |
| | taa-3′ | | | |
| Probe | TET-5′-tgaatgccacaact | 29 | 1474 | 329 |
| | ttatcaaagtatttg-3′- | | | |
| | TAMRA | | | |
| Reverse | 5′-aaaaccatctcatcgttt | 22 | 1449 | 330 |
| | cttg-3′ | | | |

TABLE MC
AI comprehensive panel v1.0

| Tissue Name | A |
|---|---|
| 110967 COPD-F | 7.5 |
| 110980 COPD-F | 12.4 |
| 110968 COPD-M | 4.4 |
| 110977 COPD-M | 13.1 |
| 110989 Emphysema-F | 4.5 |
| 110992 Emphysema-F | 1.8 |
| 110993 Emphysema-F | 8.0 |
| 110994 Emphysema-F | 3.1 |
| 110995 Emphysema-F | 4.5 |
| 110996 Emphysema-F | 0.1 |
| 110997 Asthma-M | 12.2 |
| 111001 Asthma-F | 8.0 |
| 111002 Asthma-F | 9.2 |
| 111003 Atopic Asthma-F | 6.4 |
| 111004 Atopic Asthma-F | 4.8 |
| 111005 Atopic Asthma-F | 4.7 |
| 111006 Atopic Asthma-F | 0.8 |
| 111417 Allergy-M | 5.6 |
| 112347 Allergy-M | 4.9 |
| 112349 Normal Lung-F | 5.4 |
| 112357 Normal Lung-F | 100.0 |
| 112354 Normal Lung-M | 49.7 |
| 112374 Crohns-F | 3.2 |
| 112389 Match Control Crohns-F | 21.2 |
| 112375 Crohns-F | 3.9 |
| 112732 Match Control Crohns-F | 1.7 |
| 112725 Crohns-M | 5.7 |
| 112387 Match Control Crohns-M | 7.3 |
| 112378 Crohns-M | 9.2 |
| 112390 Match Control Crohns-M | 13.0 |
| 112726 Crohns-M | 16.7 |
| 112731 Match Control Crohns-M | 6.3 |
| 112380 Ulcer Col-F | 5.6 |
| 112734 Match Control Ulcer Col-F | 1.4 |
| 112384 Ulcer Col-F | 12.2 |
| 112737 Match Control Ulcer Col-F | 12.7 |
| 112386 Ulcer Col-F | 3.0 |
| 112738 Match Control Ulcer Col-F | 9.4 |
| 112381 Ulcer Col-M | 2.9 |
| 112735 Match Control Ulcer Col-M | 29.1 |
| 112382 Ulcer Col-M | 33.0 |
| 112394 Match Control Ulcer Col-M | 5.2 |
| 112383 Ulcer Col-M | 4.1 |
| 112736 Match Control Ulcer Col-M | 11.2 |
| 112423 Psoriasis-F | 14.4 |
| 112427 Match Control Psoriasis-F | 13.6 |
| 112418 Psoriasis-M | 7.0 |
| 112723 Match Control Psoriasis-M | 67.4 |
| 112419 Psoriasis-M | 14.1 |
| 112424 Match Control Psoriasis-M | 7.9 |
| 112420 Psoriasis-M | 9.2 |
| 112425 Match Control Psoriasis-M | 15.5 |
| 104689 (MF) OA Bone-Backus | 77.9 |
| 104690 (MF) Adj “Normal” Bone-Backus | 52.5 |
| 104691 (MF) OA Synovium-Backus | 60.3 |
| 104692 (BA) OA Cartilage-Backus | 13.4 |
| 104694 (BA) OA Bone-Backus | 58.6 |
| 104695 (BA) Adj “Normal” Bone-Backus | 40.6 |
| 104696 (BA) OA Synovium-Backus | 50.0 |
| 104700 (SS) OA Bone-Backus | 50.7 |
| 104701 (SS) Adj “Normal” Bone-Backus | 49.0 |
| 104702 (SS) OA Synovium-Backus | 30.8 |
| 117093 OA Cartilage Rep7 | 7.3 |
| 112672 OA Bone5 | 9.8 |
| 112673 OA Synovium5 | 5.5 |
| 112674 OA Synovial Fluid cells5 | 3.3 |
| 117100 OA Cartilage Rep14 | 1.5 |
| 112756 OA Bone9 | 9.9 |
| 112757 OA Synovium9 | 13.8 |
| 112758 OA Synovial Fluid Cells9 | 4.5 |
| 117125 RA Cartilage Rep2 | 9.2 |
| 113492 Bone2 RA | 19.3 |
| 113493 Synovium2 RA | 17.7 |
| 113494 Syn Fluid Cells RA | 24.0 |
| 113499 Cartilage4 RA | 36.3 |
| 113500 Bone4 RA | 43.5 |
| 113501 Synovium4 RA | 34.9 |
| 113502 Syn Fluid Cells4 RA | 15.4 |
| 113495 Cartilage3 RA | 27.2 |
| 113496 Bone3 RA | 19.3 |
| 113497 Synovium3 RA | 10.6 |
| 113498 Syn Fluid Cells3 RA | 27.4 |
| 117106 Normal Cartilage Rep20 | 0.6 |
| 113663 Bone3 Normal | 6.9 |
| 113664 Synovium3 Normal | 1.8 |
| 113665 Syn Fluid Cells3 Normal | 4.3 |
| 117107 Normal Cartilage Rep22 | 4.6 |
| 113667 Bone4 Normal | 7.6 |
| 113668 Synovium4 Normal | 6.8 |
| 113669 Syn Fluid Cells4 Normal | 6.7 |

Column A - Rel. Exp. (%) Ag1492, Run 248065288

TABLE MD
Panel 1.3D
Column A - Rel. Exp. (%) Ag1492, Run 165529502
Column B - Rel. Exp. (%) Ag2441, Run 159616039
Column C - Rel. Exp. (%) Ag2441, Run 165534561

| Tissue Name | A | B | C |
|---|---|---|---|
| Liver adenocarcinoma | 0.0 | 0.0 | 0.0 |
| Pancreas | 4.5 | 1.4 | 4.5 |
| Pancreatic ca. CAPAN 2 | 0.0 | 0.0 | 0.0 |
| Adrenal gland | 2.8 | 0.8 | 2.5 |
| Thyroid | 4.9 | 3.3 | 2.1 |
| Salivary gland | 2.0 | 1.1 | 2.3 |
| Pituitary gland | 9.2 | 6.6 | 2.9 |
| Brain (fetal) | 44.4 | 12.1 | 26.4 |
| Brain (whole) | 100.0 | 20.0 | 81.2 |
| Brain (amygdala) | 27.7 | 16.8 | 25.5 |
| Brain (cerebellum) | 42.3 | 8.8 | 27.2 |
| Brain (hippocampus) | 50.0 | 77.9 | 26.2 |
| Brain (substantia nigra) | 42.9 | 7.6 | 24.8 |
| Brain (thalamus) | 52.1 | 15.3 | 30.1 |
| Cerebral Cortex | 43.2 | 70.2 | 23.0 |
| Spinal cord | 18.7 | 8.0 | 14.2 |
| glio/astro U87-MG | 2.4 | 3.2 | 1.5 |
| glio/astro U-118-MG | 77.4 | 100.0 | 100.0 |
| astrocytoma SW1783 | 0.0 | 0.6 | 0.3 |
| neuro*; met SK-N-AS | 1.3 | 9.8 | 2.2 |
| astrocytoma SF-539 | 0.0 | 0.0 | 0.0 |
| astrocytoma SNB-75 | 5.4 | 3.8 | 6.2 |
| glioma SNB-19 | 3.3 | 4.6 | 3.9 |
| glioma U251 | 15.8 | 3.3 | 10.6 |
| glioma SF-295 | 10.2 | 10.7 | 14.3 |
| Heart (fetal) | 4.4 | 10.8 | 2.5 |
| Heart | 4.1 | 1.3 | 5.1 |
| Skeletal muscle (fetal) | 4.1 | 35.8 | 1.3 |
| Skeletal muscle | 34.2 | 4.5 | 28.3 |
| Bone marrow | 1.3 | 1.7 | 0.3 |
| Thymus | 1.3 | 0.7 | 1.7 |
| Spleen | 1.2 | 1.1 | 2.5 |
| Lymph node | 3.9 | 1.8 | 3.0 |
| Colorectal | 15.2 | 10.4 | 6.3 |
| Stomach | 6.0 | 2.3 | 4.3 |
| Small intestine | 19.3 | 10.6 | 10.7 |
| Colon ca. SW480 | 0.0 | 0.0 | 0.0 |
| Colon ca.* SW620(SW480 met) | 0.2 | 0.0 | 0.0 |
| Colon ca. HT29 | 0.0 | 0.0 | 0.0 |
| Colon ca. HCT-116 | 0.5 | 0.0 | 0.0 |
| Colon ca. CaCo-2 | 0.5 | 0.6 | 0.0 |
| Colon ca. tissue(ODO3866) | 0.2 | 1.9 | 2.1 |
| Colon ca. HCC-2998 | 0.0 | 0.0 | 0.0 |
| Gastric ca.* (liver met) | 0.0 | 0.0 | 0.0 |
| NCI-N87 | | | |
| Bladder | 3.4 | 1.7 | 3.4 |
| Trachea | 1.5 | 2.4 | 1.4 |
| Kidney | 13.0 | 4.2 | 20.6 |
| Kidney (fetal) | 14.9 | 7.8 | 12.2 |
| Renal ca. 786-0 | 3.9 | 1.8 | 1.2 |
| Renal ca. A498 | 0.6 | 0.3 | 0.0 |
| Renal ca. RXF 393 | 9.8 | 2.2 | 6.8 |
| Renal ca. ACHN | 0.0 | 0.0 | 0.0 |
| Renal ca. UO-31 | 0.2 | 0.2 | 0.7 |
| Renal ca. TK-10 | 0.0 | 0.0 | 0.0 |
| Liver | 4.4 | 0.9 | 2.9 |
| Liver (fetal) | 4.4 | 1.3 | 3.1 |
| Liver ca. (hepatoblast) HepG2 | 0.0 | 0.0 | 0.0 |
| Lung | 5.0 | 11.4 | 4.4 |
| Lung (fetal) | 7.3 | 7.4 | 13.5 |
| Lung ca. (small cell) LX-1 | 0.0 | 0.0 | 0.0 |
| Lung ca. (small cell) NCI-H69 | 4.0 | 23.0 | 18.4 |
| Lung ca. (s. cell var.) SHP-77 | 14.7 | 21.5 | 15.6 |
| Lung ca. (large cell)NCI-H460 | 2.2 | 0.3 | 0.7 |
| Lung ca. (non-sm. cell) A549 | 0.0 | 0.0 | 0.0 |
| Lung ca. (non-s. cell) NCI-H23 | 0.0 | 0.0 | 0.0 |
| Lung ca. (non-s. cell) HOP-62 | 1.3 | 0.9 | 1.4 |
| Lung ca. (non-s. cl) NCI-H522 | 0.0 | 0.0 | 0.0 |
| Lung ca. (squam.) SW 900 | 5.0 | 4.1 | 5.1 |
| Lung ca. (squam.) NCI-H596 | 9.9 | 7.1 | 13.7 |
| Mammary gland | 20.2 | 10.1 | 6.9 |
| Breast ca.* (pl. ef) MCF-7 | 0.0 | 0.0 | 0.1 |
| Breast ca.* (pl. ef) | 0.0 | 0.3 | 0.3 |
| MDA-MB-231 | | | |
| Breast ca.* (pl. ef) T47D | 0.0 | 0.0 | 0.0 |
| Breast ca. BT-549 | 0.0 | 0.0 | 0.0 |
| Breast ca. MDA-N | 0.6 | 2.3 | 0.3 |
| Ovary | 9.8 | 20.6 | 3.8 |
| Ovarian ca. OVCAR-3 | 5.6 | 3.3 | 6.3 |
| Ovarian ca. OVCAR-4 | 0.0 | 0.0 | 0.0 |
| Ovarian ca. OVCAR-5 | 0.3 | 0.0 | 0.0 |
| Ovarian ca. OVCAR-8 | 0.8 | 1.8 | 0.7 |
| Ovarian ca. IGROV-1 | 0.0 | 0.0 | 0.0 |
| Ovarian ca.* (ascites) | 0.0 | 0.0 | 0.0 |
| SK-OV-3 | | | |
| Uterus | 3.3 | 0.9 | 3.7 |
| Placenta | 17.8 | 14.8 | 8.1 |
| Prostate | 3.7 | 0.6 | 1.4 |
| Prostate ca.* (bone met)PC-3 | 1.4 | 2.2 | 4.8 |
| Testis | 1.8 | 0.8 | 1.3 |
| Melanoma Hs688(A).T | 0.0 | 0.0 | 0.1 |
| Melanoma* (met) Hs688(B).T | 0.0 | 0.5 | 0.6 |
| Melanoma UACC-62 | 5.7 | 0.9 | 2.3 |
| Melanoma M14 | 6.1 | 1.3 | 9.2 |
| Melanoma LOX IMVI | 0.0 | 0.0 | 0.0 |
| Melanoma* (met) SK-MEL-5 | 2.0 | 2.3 | 1.3 |
| Adipose | 8.3 | 5.7 | 9.8 |

TABLE ME
Panel 2D

| Tissue Name | A |
|---|---|
| Normal Colon | 48.6 |
| CC Well to Mod Diff (ODO3866) | 0.6 |
| CC Margin (ODO3866) | 6.6 |
| CC Gr.2 rectosigmoid (ODO3868) | 0.9 |
| CC Margin (ODO3868) | 1.2 |
| CC Mod Diff (ODO3920) | 0.5 |
| CC Margin (ODO3920) | 9.1 |
| CC Gr.2 ascend colon (ODO3921) | 10.9 |
| CC Margin (ODO3921) | 6.7 |
| CC from Partial Hepatectomy (ODO4309) Mets | 2.0 |
| Liver Margin (ODO4309) | 3.5 |
| Colon mets to lung (OD04451-01) | 0.6 |
| Lung Margin (OD04451-02) | 3.5 |
| Normal Prostate 6546-1 | 1.4 |
| Prostate Cancer (OD04410) | 2.9 |
| Prostate Margin (OD04410) | 8.0 |
| Prostate Cancer (OD04720-01) | 6.6 |
| Prostate Margin (OD04720-02) | 13.3 |
| Normal Lung 061010 | 14.4 |
| Lung Met to Muscle (ODO4286) | 0.1 |
| Muscle Margin (ODO4286) | 4.5 |
| Lung Malignant Cancer (OD03126) | 4.3 |
| Lung Margin (OD03126) | 15.0 |
| Lung Cancer (OD04404) | 8.4 |
| Lung Margin (OD04404) | 3.7 |
| Lung Cancer (OD04565) | 1.1 |
| Lung Margin (OD04565) | 4.7 |
| Lung Cancer (OD04237-01) | 1.2 |
| Lung Margin (OD04237-02) | 5.6 |
| Ocular Mel Met to Liver (ODO4310) | 2.7 |
| Liver Margin (ODO4310) | 3.0 |
| Melanoma Mets to Lung (OD04321) | 0.7 |
| Lung Margin (OD04321) | 8.0 |
| Normal Kidney | 100.0 |
| Kidney Ca, Nuclear grade 2 (OD04338) | 3.6 |
| Kidney Margin (OD04338) | 32.5 |
| Kidney Ca Nuclear grade ½ (OD04339) | 0.5 |
| Kidney Margin (OD04339) | 26.8 |
| Kidney Ca, Clear cell type (OD04340) | 3.8 |
| Kidney Margin (OD04340) | 35.4 |
| Kidney Ca, Nuclear grade 3 (OD04348) | 0.2 |
| Kidney Margin (OD04348) | 15.7 |
| Kidney Cancer (OD04622-01) | 1.1 |
| Kidney Margin (OD04622-03) | 4.2 |
| Kidney Cancer (OD04450-01) | 8.0 |
| Kidney Margin (OD04450-03) | 25.0 |
| Kidney Cancer 8120607 | 0.6 |
| Kidney Margin 8120608 | 2.6 |
| Kidney Cancer 8120613 | 0.4 |
| Kidney Margin 8120614 | 11.5 |
| Kidney Cancer 9010320 | 1.7 |
| Kidney Margin 9010321 | 11.3 |
| Normal Uterus | 0.9 |
| Uterus Cancer 064011 | 3.4 |
| Normal Thyroid | 3.9 |
| Thyroid Cancer 064010 | 2.0 |
| Thyroid Cancer A302152 | 0.6 |
| Thyroid Margin A302153 | 10.8 |
| Normal Breast | 12.2 |
| Breast Cancer (OD04566) | 0.4 |
| Breast Cancer (OD04590-01) | 7.3 |
| Breast Cancer Mets (OD04590-03) | 4.8 |
| Breast Cancer Metastasis (OD04655-05) | 3.6 |
| Breast Cancer 064006 | 2.0 |
| Breast Cancer 1024 | 5.4 |
| Breast Cancer 9100266 | 2.1 |
| Breast Margin 9100265 | 7.4 |
| Breast Cancer A209073 | 8.5 |
| Breast Margin A209073 | 13.8 |
| Normal Liver | 2.7 |
| Liver Cancer 064003 | 0.1 |
| Liver Cancer 1025 | 2.3 |
| Liver Cancer 1026 | 0.7 |
| Liver Cancer 6004-T | 4.0 |
| Liver Tissue 6004-N | 0.3 |
| Liver Cancer 6005-T | 0.5 |
| Liver Tissue 6005-N | 0.6 |
| Normal Bladder | 4.7 |
| Bladder Cancer 1023 | 0.1 |
| Bladder Cancer A302173 | 4.9 |
| Bladder Cancer (OD04718-01) | 0.0 |
| Bladder Normal Adjacent (OD04718-03) | 2.8 |
| Normal Ovary | 7.2 |
| Ovarian Cancer 064008 | 6.8 |
| Ovarian Cancer (OD04768-07) | 0.2 |
| Ovary Margin (OD04768-08) | 1.0 |
| Normal Stomach | 6.3 |
| Gastric Cancer 9060358 | 1.6 |
| Stomach Margin 9060359 | 2.1 |
| Gastric Cancer 9060395 | 4.2 |
| Stomach Margin 9060394 | 4.2 |
| Gastric Cancer 9060397 | 1.6 |
| Stomach Margin 9060396 | 0.5 |
| Gastric Cancer 064005 | 8.9 |

Column A - Rel. Exp. (%) Ag2441, Run 159616246

TABLE MF
Panel 4D
Column A - Rel. Exp. (%) Ag1492, Run 162778150
Column B - Rel. Exp. (%) Ag2441, Run 159616279

| Tissue Name | A | B |
|---|---|---|
| Secondary Th1 act | 0.0 | 0.0 |
| Secondary Th2 act | 0.0 | 0.0 |
| Secondary Tr1 act | 0.0 | 0.0 |
| Secondary Th1 rest | 0.0 | 0.0 |
| Secondary Th2 rest | 0.0 | 0.0 |
| Secondary Tr1 rest | 0.0 | 0.0 |
| Primary Th1 act | 0.0 | 0.0 |
| Primary Th2 act | 0.0 | 0.0 |
| Primary Tr1 act | 0.0 | 0.0 |
| Primary Th1 rest | 0.0 | 0.0 |
| Primary Th2 rest | 0.0 | 0.0 |
| Primary Tr1 rest | 0.0 | 0.0 |
| CD45RA CD4 lymphocyte act | 0.4 | 0.6 |
| CD45RO CD4 lymphocyte act | 0.0 | 0.0 |
| CD8 lymphocyte act | 0.0 | 0.0 |
| Secondary CD8 lymphocyte rest | 0.0 | 0.0 |
| Secondary CD8 lymphocyte act | 0.0 | 0.0 |
| CD4 lymphocyte none | 0.0 | 0.0 |
| 2ry Th1/Th2/Tr1_anti-CD95 CH11 | 0.0 | 0.0 |
| LAK cells rest | 0.0 | 0.0 |
| LAK cells IL-2 | 0.0 | 0.0 |
| LAK cells IL-2 + IL-12 | 0.0 | 0.0 |
| LAK cells IL-2 + IFN gamma | 0.0 | 0.0 |
| LAK cells IL-2 + IL-18 | 0.0 | 0.0 |
| LAK cells PMA/ionomycin | 0.0 | 0.0 |
| NK Cells IL-2 rest | 0.0 | 0.0 |
| Two Way MLR 3 day | 0.0 | 0.0 |
| Two Way MLR 5 day | 0.0 | 0.0 |
| Two Way MLR 7 day | 0.0 | 0.0 |
| PBMC rest | 0.0 | 0.0 |
| PBMC PWM | 0.2 | 0.0 |
| PBMC PHA-L | 0.0 | 0.0 |
| Ramos (B cell) none | 0.0 | 0.0 |
| Ramos (B cell) ionomycin | 0.0 | 0.0 |
| B lymphocytes PWM | 0.3 | 0.0 |
| B lymphocytes CD40L and IL-4 | 0.4 | 0.3 |
| EOL-1 dbcAMP | 0.0 | 0.0 |
| EOL-1 dbcAMP PMA/ionomycin | 0.0 | 0.0 |
| Dendritic cells none | 0.0 | 0.0 |
| Dendritic cells LPS | 0.1 | 0.0 |
| Dendritic cells anti-CD40 | 0.0 | 0.1 |
| Monocytes rest | 0.0 | 0.0 |
| Monocytes LPS | 0.0 | 0.0 |
| Macrophages rest | 0.0 | 0.3 |
| Macrophages LPS | 0.0 | 0.0 |
| HUVEC none | 11.6 | 5.8 |
| HUVEC starved | 19.3 | 18.0 |
| HUVEC IL-1beta | 12.5 | 9.6 |
| HUVEC IFN gamma | 5.5 | 6.2 |
| HUVEC TNF alpha + IFN gamma | 3.6 | 1.9 |
| HUVEC TNF alpha + IL4 | 5.6 | 5.0 |
| HUVEC IL-11 | 8.5 | 6.5 |
| Lung Microvascular EC none | 0.4 | 0.1 |
| Lung Microvascular EC TNFalpha + | 0.0 | 0.0 |
| IL-1beta | | |
| Microvascular Dermal EC none | 0.1 | 0.1 |
| Microsvasular Dermal EC TNFalpha + | 0.0 | 0.0 |
| IL-1beta | | |
| Bronchial epithelium TNFalpha + | 2.1 | 2.7 |
| IL1beta | | |
| Small airway epithelium none | 0.5 | 0.5 |
| Small airway epithelium TNFalpha + | 1.1 | 0.8 |
| IL-1beta | | |
| Coronery artery SMC rest | 1.2 | 1.5 |
| Coronery artery SMC TNFalpha + | 0.4 | 0.3 |
| IL-1beta | | |
| Astrocytes rest | 1.5 | 1.3 |
| Astrocytes TNFalpha + IL-1beta | 0.0 | 0.1 |
| KU-812 (Basophil) rest | 0.0 | 0.0 |
| KU-812 (Basophil) PMA/ionomycin | 0.0 | 0.0 |
| CCD1106 (Keratinocytes) none | 0.9 | 0.5 |
| CCD1106 (Keratinocytes) TNFalpha + | 0.0 | 0.1 |
| IL-1beta | | |
| Liver cirrhosis | 5.7 | 2.6 |
| Lupus kidney | 4.5 | 2.4 |
| NCI-H292 none | 5.3 | 4.9 |
| NCI-H292 IL-4 | 3.0 | 4.9 |
| NCI-H292 IL-9 | 5.2 | 5.3 |
| NCI-H292 IL-13 | 2.5 | 1.6 |
| NCI-H292 IFN gamma | 1.8 | 0.0 |
| HPAEC none | 3.7 | 5.2 |
| HPAEC TNF alpha + IL-1 beta | 6.5 | 4.3 |
| Lung fibroblast none | 16.2 | 10.4 |
| Lung fibroblast TNF alpha + IL-1 beta | 81.2 | 65.5 |
| Lung fibroblast IL-4 | 12.0 | 12.9 |
| Lung fibroblast IL-9 | 22.2 | 13.3 |
| Lung fibroblast IL-13 | 7.9 | 5.9 |
| Lung fibroblast IFN gamma | 10.2 | 7.8 |
| Dermal fibroblast CCD1070 rest | 3.3 | 2.4 |
| Dermal fibroblast CCD1070 TNF alpha | 2.5 | 4.5 |
| Dermal fibroblast CCD1070 IL-1 beta | 6.0 | 5.1 |
| Dermal fibroblast IFN gamma | 2.1 | 0.7 |
| Dermal fibroblast IL-4 | 9.7 | 7.7 |
| IBD Colitis 2 | 1.7 | 0.2 |
| IBD Crohn's | 13.7 | 7.7 |
| Colon | 98.6 | 95.3 |
| Lung | 17.2 | 16.0 |
| Thymus | 100.0 | 100.0 |
| Kidney | 6.3 | 4.2 |

AI_comprehensive panel_v1.0 Summary: Ag1492 Highest expression of the CG59253-01 and CG59253-02 genes was detected in normal lung (CT=27.6). Significant expression of these genes was detected in samples derived from normal and orthoarthitis/rheumatoid arthritis bone, cartilage, synovium and synovial fluid samples, normal lung, COPD lung, emphysema, atopic asthma, asthma, allergy, Crohn's disease (normal matched control and diseased), ulcerative colitis (normal matched control and diseased), and psoriasis (normal matched control and diseased). Therapeutic modulation of the activity of these gene variants or their protein products is useful in the treatment of autoimmune and inflammatory disorders including psoriasis, allergy, asthma, inflammatory bowel disease, rheumatoid arthritis and osteoarthritis
Panel 1.3D Summary: Ag1492/Ag2441 The CG59253-01 and CG59253-02 genes encode a semaphorin homolog that had brain-preferential expression. Highest expression of these variants was seen in the brain and a brain cancer cell line (CTs=28-29). Semaphorins can act as axon guidance proteins, specifically as chemorepellents that inhibit CNS regenerative capacity. Manipulation of levels of these gene variants or their protein products is useful in inducing a compensatory synaptogenic response to neuronal death in Alzheimer's disease, Parkinson's disease, Huntington's disease, spinocerebellar ataxia, progressive supranuclear palsy, multiple sclerosis, ALS, head trauma, stroke, or any other disease/condition associated with neuronal loss. Therapeutic modulation of the activity of these gene variants or their protein products using nucleic acid, protein, antibody or small molecule drugs is useful in the treatment of brain cancer.

This gene was also moderately expressed in several metabolic tissues, including pancreas, adrenal, thyroid, pituitary, adult and fetal heart, adult and fetal skeletal muscle, adult and fetal liver, and adipose. Gene or protein expression levels are important for the pathogenesis, diagnosis, and/or treatment of metabolic diseases including obesity and Types 1 and 2 diabetes.

Panel 2D Summary: Ag2441 Highest expression of the CG59253-01 and CG59253-02 gene variants was detected in normal kidney (CT=27.6). These variants were more highly expressed in normal kidney samples relative to the matched kidney cancers. Gene or protein levels are useful to distinguish normal kidney from kidney cancer. These genes encode variants of the semaphorin SEMA6D protein. The semaphorin family of proteins is characterized as cell surface receptors for their ligands, the pillins, and is involved largely in cell guidance (Tamagnone L, Comoglio P M. Signaling by semaphorin receptors: cell guidance and beyond. Trends Cell Biol 2000 September; 10(9):377-83). Semaphorins have been implicated in general invasive growth and potentially even tube formation (Comoglio P M, Tamagnone L, Boccaccio C. Plasminogen-related growth factor and semaphorin receptors: a gene superfamily controlling invasive growth. Exp Cell Res Nov. 25, 1999;253(1):88-99). Thus, semaphorins are likely agents to promote the differentiation of cells. Normal kidney cells undergo a great deal of tubular morphogenesis. Therefore, the extracellular domain of these protein products may act to promote growth arrest and differentiation of the cancer cells through interaction with a membrane bound ligand or ligand complexed with plexins. Therapeutic modulation of the activity of these gene variants or their protein products using nucleic acid, protein, antibody or small molecule drugs is of use in the treatment of kidney cancer.

Panel 4D Summary: Ag1492/2441 Highest expression of the CG59253-01 and CG59253-02 gene variants was detected in thymus (CTs=27-28). Significant expression of these variants was also seen activated lung fibroblasts cells, HUVEC, HPAEC, activated bronchial epithelium, NCI-H292 cell, dermal fibroblasts, IBD Crohn's, liver cirrhosis and lupus samples, normal tissues colon and thymus. Therapeutic modulation of the activity of these gene variants or their protein products is useful to reduce or eliminate the symptoms of chronic obstructive pulmonary disease, asthma, emphysema, and ulcerative colitis

N. CG95430-01: AdipoQ-like

Expression of gene CG95430-01 was assessed using the primer-probe set Ag4020, described in Table NA. Results of the RTQ-PCR runs are shown in Tables NB, NC, ND, NE and NF.

TABLE NA
Probe Name Ag4020

| | | | Start | SEQ ID |
|---|---|---|---|---|
| Primers | Sequences | Length | Position | No |
| Forward | 5′-cacattgctggggtctat | 22 | 458 | 331 |
| | tact-3′ | | | |
| Probe | TET-5′-tcacctaccacatc | 27 | 480 | 332 |
| | actgttttctcca-3′- | | | |
| | TAMRA | | | |
| Reverse | 5′-ttttgaccaaagacacct | 22 | 512 | 333 |
| | gaac-3′ | | | |

TABLE NB
CNS neurodegeneration v1.0

| Tissue Name | A |
|---|---|
| AD 1 Hippo | 40.1 |
| AD 2 Hippo | 19.3 |
| AD 3 Hippo | 22.2 |
| AD 4 Hippo | 26.4 |
| AD 5 Hippo | 4.9 |
| AD 6 Hippo | 100.0 |
| Control 2 Hippo | 26.8 |
| Control 4 Hippo | 21.2 |
| Control (Path) 3 Hippo | 14.9 |
| AD 1 Temporal Ctx | 11.1 |
| AD 2 Temporal Ctx | 10.3 |
| AD 3 Temporal Ctx | 6.0 |
| AD 4 Temporal Ctx | 15.8 |
| AD 5 Inf Temporal Ctx | 9.8 |
| AD 5 Sup Temporal Ctx | 31.2 |
| AD 6 Inf Temporal Ctx | 13.7 |
| AD 6 Sup Temporal Ctx | 8.0 |
| Control 1 Temporal Ctx | 2.4 |
| Control 2 Temporal Ctx | 3.2 |
| Control 3 Temporal Ctx | 8.9 |
| Control 3 Temporal Ctx | 0.8 |
| Control (Path) 1 Temporal Ctx | 6.0 |
| Control (Path) 2 Temporal Ctx | 4.3 |
| Control (Path) 3 Temporal Ctx | 7.6 |
| Control (Path) 4 Temporal Ctx | 7.7 |
| AD 1 Occipital Ctx | 5.8 |
| AD 2 Occipital Ctx (Missing) | 12.5 |
| AD 3 Occipital Ctx | 4.8 |
| AD 4 Occipital Ctx | 3.9 |
| AD 5 Occipital Ctx | 3.5 |
| AD 6 Occipital Ctx | 3.4 |
| Control 1 Occipital Ctx | 7.3 |
| Control 2 Occipital Ctx | 3.9 |
| Control 3 Occipital Ctx | 6.7 |
| Control 4 Occipital Ctx | 1.8 |
| Control (Path) 1 Occipital Ctx | 12.5 |
| Control (Path) 2 Occipital Ctx | 3.1 |
| Control (Path) 3 Occipital Ctx | 2.8 |
| Control (Path) 4 Occipital Ctx | 6.7 |
| Control 1 Parietal Ctx | 3.3 |
| Control 2 Parietal Ctx | 7.5 |
| Control 3 Parietal Ctx | 9.5 |
| Control (Path) 1 Parietal Ctx | 8.4 |
| Control (Path) 2 Parietal Ctx | 6.1 |
| Control (Path) 3 Parietal Ctx | 6.0 |
| Control (Path) 4 Parietal Ctx | 4.7 |

Column A - Rel. Exp. (%) Ag4020, Run 212393803

TABLE NC
Oncology cell line screening panel v3.1

| Tissue Name | A |
|---|---|
| Daoy Medulloblastoma/Cerebellum | 1.1 |
| TE671 Medulloblastom/Cerebellum | 0.0 |
| D283 Med Medulloblastoma/Cerebellum | 3.3 |
| PFSK-1 Primitive Neuroectodermal/Cerebellum | 0.0 |
| XF-498_CNS | 10.3 |
| SNB-78_CNS/glioma | 0.0 |
| SF-268_CNS/glioblastoma | 18.7 |
| T98G_Glioblastoma | 5.4 |
| SK-N-SH_Neuroblastoma (metastasis) | 2.9 |
| SF-295_CNS/glioblastoma | 0.0 |
| Cerebellum | 6.7 |
| Cerebellum | 2.5 |
| NCI-H292_Mucoepidermoid lung ca. | 7.7 |
| DMS-114_Small cell lung cancer | 8.5 |
| DMS-79_Small cell lung cancer/neuroendocrine | 1.6 |
| NCI-H146_Small cell lung cancer/neuroendocrine | 4.5 |
| NCI-H526_Small cell lung cancer/neuroendocrine | 5.5 |
| NCI-N417_Small cell lung cancer/neuroendocrine | 1.7 |
| NCI-H82_Small cell lung cancer/neuroendocrine | 9.3 |
| NCI-H157_Squamous cell lung cancer (metastasis) | 0.0 |
| NCI-H1155_Large cell lung cancer/neuroendocrine | 6.2 |
| NCI-H1299_Large cell lung cancer/neuroendocrine | 5.2 |
| NCI-H727_Lung carcinoid | 0.0 |
| NCI-UMC-11_Lung carcinoid | 1.2 |
| LX-1_Small cell lung cancer | 1.6 |
| Colo-205_Colon cancer | 18.6 |
| KM12_Colon cancer | 11.0 |
| KM20L2_Colon cancer | 1.1 |
| NCI-H716_Colon cancer | 6.3 |
| SW-48_Colon adenocarcinoma | 3.7 |
| SW1116_Colon adenocarcinoma | 11.7 |
| LS 174T_Colon adenocarcinoma | 27.9 |
| SW-948_Colon adenocarcinoma | 4.2 |
| SW-480_Colon adenocarcinoma | 2.0 |
| NCI-SNU-5_Gastric ca. | 9.9 |
| KATO III_Stomach | 9.3 |
| NCI-SNU-16_Gastric ca. | 4.7 |
| NCI-SNU-1_Gastric ca. | 9.2 |
| RF-1_Gastric adenocarcinoma | 1.2 |
| RF-48_Gastric adenocarcinoma | 7.6 |
| MKN-45_Gastric ca. | 20.3 |
| NCI-N87_Gastric ca. | 14.4 |
| OVCAR-5_Ovarian ca. | 0.0 |
| RL95-2_Uterine carcinoma | 2.3 |
| HelaS3_Cervical adenocarcinoma | 10.1 |
| Ca Ski_Cervical epidermoid carcinoma (metastasis) | 7.9 |
| ES-2_Ovarian clear cell carcinoma | 2.4 |
| Ramos/6 h stim_Stimulated with PMA/ionomycin 6 h | 0.0 |
| Ramos/14 h stim_Stimulated with PMA/ionomycin 14 h | 0.0 |
| MEG-01_Chronic myelogenous leukemia (megokaryoblast) | 1.4 |
| Raji_Burkitt's lymphoma | 0.0 |
| Daudi_Burkitt's lymphoma | 0.0 |
| U266_B-cell plasmacytoma/myeloma | 0.0 |
| CA46_Burkitt's lymphoma | 1.2 |
| RL_non-Hodgkin's B-cell lymphoma | 0.0 |
| JM1_pre-B-cell lymphoma/leukemia | 2.0 |
| Jurkat_T cell leukemia | 7.5 |
| TF-1_Erythroleukemia | 2.3 |
| HUT 78_T-cell lymphoma | 1.7 |
| U937_Histiocytic lymphoma | 9.2 |
| KU-812_Myelogenous leukemia | 7.7 |
| 769-P_Clear cell renal ca. | 0.0 |
| Caki-2_Clear cell renal ca. | 13.5 |
| SW 839_Clear cell renal ca. | 0.0 |
| G401_Wilms' tumor | 0.9 |
| Hs766T_Pancreatic ca. (LN metastasis) | 21.3 |
| CAPAN-1_Pancreatic adenocarcinoma (liver metastasis) | 0.0 |
| SU86.86_Pancreatic carcinoma (liver metastasis) | 0.9 |
| BxPC-3_Pancreatic adenocarcinoma | 7.7 |
| HPAC_Pancreatic adenocarcinoma | 1.0 |
| MIA PaCa-2_Pancreatic ca. | 0.0 |
| CFPAC-1_Pancreatic ductal adenocarcinoma | 71.2 |
| PANC-1_Pancreatic epithelioid ductal ca. | 3.0 |
| T24_Bladder ca. (transitional cell) | 0.0 |
| 5637_Bladder ca. | 1.9 |
| HT-1197_Bladder ca. | 0.0 |
| UM-UC-3_Bladder ca. (transitional cell) | 0.0 |
| A204_Rhabdomyosarcoma | 6.2 |
| HT-1080_Fibrosarcoma | 30.6 |
| MG-63_Osteosarcoma (bone) | 4.2 |
| SK-LMS-1_Leiomyosarcoma (vulva) | 7.3 |
| SJRH30_Rhabdomyosarcoma (met to bone marrow) | 4.4 |
| A431_Epidermoid ca. | 100.0 |
| WM266-4_Melanoma | 1.9 |
| DU 145_Prostate | 0.7 |
| MDA-MB-468_Breast adenocarcinoma | 0.0 |
| SSC-4_Tongue | 1.5 |
| SSC-9_Tongue | 0.0 |
| SSC-15_Tongue | 1.2 |
| CAL 27_Squamous cell ca. of tongue | 2.9 |

Column A - Rel. Exp. (%) Ag4020, Run 22254637

TABLE ND
Panel 4.1D

| Tissue Name | A |
|---|---|
| Secondary Th1 act | 2.4 |
| Secondary Th2 act | 10.7 |
| Secondary Tr1 act | 1.5 |
| Secondary Th1 rest | 1.9 |
| Secondary Th2 rest | 2.5 |
| Secondary Tr1 rest | 0.0 |
| Primary Th1 act | 1.5 |
| Primary Th2 act | 3.6 |
| Primary Tr1 act | 1.6 |
| Primary Th1 rest | 1.3 |
| Primary Th2 rest | 0.6 |
| Primary Tr1 rest | 0.0 |
| CD45RA CD4 lymphocyte act | 3.4 |
| CD45RO CD4 lymphocyte act | 3.2 |
| CD8 lymphocyte act | 1.4 |
| Secondary CD8 lymphocyte rest | 4.9 |
| Secondary CD8 lymphocyte act | 0.0 |
| CD4 lymphocyte none | 0.0 |
| 2ry Th1/Th2/Tr1_anti-CD95 CH11 | 0.0 |
| LAK cells rest | 2.1 |
| LAK cells IL-2 | 6.3 |
| LAK cells IL-2 + IL-12 | 2.3 |
| LAK cells IL-2 + IFN gamma | 2.3 |
| LAK cells IL-2 + IL-18 | 2.4 |
| LAK cells PMA/ionomycin | 0.6 |
| NK Cells IL-2 rest | 6.0 |
| Two Way MLR 3 day | 1.6 |
| Two Way MLR 5 day | 0.0 |
| Two Way MLR 7 day | 0.0 |
| PBMC rest | 1.1 |
| PBMC PWM | 0.0 |
| PBMC PHA-L | 0.0 |
| Ramos (B cell) none | 0.0 |
| Ramos (B cell) ionomycin | 0.0 |
| IB lymphocytes PWM | 0.0 |
| B lymphocytes CD40L and IL-4 | 0.0 |
| EOL-1 dbcAMP | 0.0 |
| EOL-1 dbcAMP PMA/ionomycin | 0.0 |
| Dendritic cells none | 2.9 |
| Dendritic cells LPS | 0.0 |
| Dendritic cells anti-CD40 | 2.6 |
| Monocytes rest | 2.0 |
| Monocytes LPS | 0.0 |
| Macrophages rest | 2.2 |
| Macrophages LPS | 2.1 |
| HUVEC none | 0.0 |
| HUVEC starved | 0.9 |
| HUVEC IL-1beta | 1.1 |
| HUVEC IFN gamma | 0.0 |
| HUVEC TNF alpha + IFN gamma | 1.0 |
| HUVEC TNF alpha + IL4 | 1.0 |
| HUVEC IL-11 | 0.7 |
| Lung Microvascular EC none | 1.0 |
| Lung Microvascular EC TNFalpha + IL-1beta | 0.0 |
| Microvascular Dermal EC none | 1.4 |
| Microsvasular Dermal EC TNFalpha + IL-1beta | 0.0 |
| Bronchial epithelium TNFalpha + IL1beta | 5.0 |
| Small airway epithelium none | 0.0 |
| Small airway epithelium TNFalpha + IL-1beta | 2.9 |
| Coronery artery SMC rest | 1.2 |
| Coronery artery SMC TNFalpha + IL-1beta | 2.5 |
| Astrocytes rest | 3.6 |
| Astrocytes TNFalpha + IL-1beta | 1.5 |
| KU-812 (Basophil) rest | 6.9 |
| KU-812 (Basophil) PMA/ionomycin | 0.0 |
| CCD1106 (Keratinocytes) none | 8.5 |
| CCD1106 (Keratinocytes) TNFalpha + IL-1beta | 1.3 |
| Liver cirrhosis | 6.6 |
| NCI-H292 none | 2.5 |
| NCI-H292 IL-4 | 0.0 |
| NCI-H292 IL-9 | 1.8 |
| NCI-H292 IL-13 | 5.9 |
| NCI-H292 IFN gamma | 0.9 |
| HPAEC none | 3.5 |
| HPAEC TNF alpha + IL-1 beta | 2.6 |
| Lung fibroblast none | 64.6 |
| Lung fibroblast TNF alpha + IL-1 beta | 1.8 |
| Lung fibroblast IL-4 | 25.5 |
| Lung fibroblast IL-9 | 14.8 |
| Lung fibroblast IL-13 | 26.1 |
| Lung fibroblast IFN gamma | 33.4 |
| Dermal fibroblast CCD1070 rest | 3.3 |
| Dermal fibroblast CCD1070 TNF alpha | 4.3 |
| Dermal fibroblast CCD1070 IL-1 beta | 2.8 |
| Dermal fibroblast IFN gamma | 1.2 |
| Dermal fibroblast IL-4 | 1.4 |
| Dermal Fibroblasts rest | 10.5 |
| Neutrophils TNFa + LPS | 0.0 |
| Neutrophils rest | 1.2 |
| Colon | 3.0 |
| Lung | 10.0 |
| Thymus | 19.6 |
| Kidney | 100.0 |

Column A - Rel. Exp. (%) Ag4020, Run 171614122

TABLE NE
Panel 5 Islet

| Tissue Name | A |
|---|---|
| 97457_Patient-02go_adipose | 22.5 |
| 97476_Patient-07sk_skeletal muscle | 41.8 |
| 97477_Patient-07ut_uterus | 5.3 |
| 97478_Patient-07pl_placenta | 2.7 |
| 99167_Bayer Patient 1 | 0.0 |
| 97482_Patient-08ut_uterus | 5.2 |
| 97483_Patient-08pl_placenta | 4.6 |
| 97486_Patient-09sk_skeletal muscle | 15.2 |
| 97487_Patient-09ut_uterus | 21.9 |
| 97488_Patient-09pl_placenta | 4.5 |
| 97492_Patient-10ut_uterus | 12.2 |
| 97493_Patient-10pl_placenta | 4.5 |
| 97495_Patient-11go_adipose | 31.4 |
| 97496_Patient-11sk_skeletal muscle | 38.2 |
| 97497_Patient-11ut_uterus | 8.4 |
| 97498_Patient-11pl_placenta | 2.2 |
| 97500_Patient-12go_adipose | 45.4 |
| 97501_Patient-12sk_skeletal muscle | 100.0 |
| 97502_Patient-12ut_uterus | 15.4 |
| 97503_Patient-12pl_placenta | 6.0 |
| 94721_Donor 2 U - A_Mesenchymal Stem Cells | 0.0 |
| 94722_Donor 2 U - B_Mesenchymal Stem Cells | 0.9 |
| 94723_Donor 2 U - C_Mesenchymal Stem Cells | 1.3 |
| 94709_Donor 2 AM - A_adipose | 2.2 |
| 94710_Donor 2 AM - B_adipose | 0.0 |
| 94711_Donor 2 AM - C_adipose | 0.8 |
| 94712_Donor 2 AD - A_adipose | 0.0 |
| 94713_Donor 2 AD - B_adipose | 5.1 |
| 94714_Donor 2 AD - C_adipose | 0.0 |
| 94742_Donor 3 U - A_Mesenchymal Stem Cells | 1.4 |
| 94743_Donor 3 U - B_Mesenchymal Stem Cells | 0.0 |
| 94730_Donor 3 AM - A_adipose | 4.3 |
| 94731_Donor 3 AM - B_adipose | 3.7 |
| 94732_Donor 3 AM - C_adipose | 0.0 |
| 94733_Donor 3 AD - A_adipose | 0.0 |
| 94734_Donor 3 AD - B_adipose | 1.6 |
| 94735_Donor 3 AD - C_adipose | 0.0 |
| 77138_Liver_HepG2untreated | 5.3 |
| 73556_Heart_Cardiac stromal cells (primary) | 0.0 |
| 81735_Small Intestine | 12.8 |
| 72409_Kidney_Proximal Convoluted Tubule | 0.0 |
| 82685_Small intestine_Duodenum | 4.4 |
| 90650_Adrenal_Adrenocortical adenoma | 0.0 |
| 72410_Kidney_HRCE | 0.0 |
| 72411_Kidney_HRE | 0.0 |
| 73139_Uterus_Uterine smooth muscle cells | 5.5 |

Column A - Rel. Exp. (%) Ag4020, Run 223675497

TABLE NF
general oncology screening panel v 2.4

| Tissue Name | A |
|---|---|
| Colon cancer 1 | 20.0 |
| Colon cancer NAT 1 | 1.1 |
| Colon cancer 2 | 11.0 |
| Colon cancer NAT 2 | 8.0 |
| Colon cancer 3 | 27.4 |
| Colon cancer NAT 3 | 49.0 |
| Colon malignant cancer 4 | 28.1 |
| Colon normal adjacent tissue 4 | 4.6 |
| Lung cancer 1 | 3.4 |
| Lung NAT 1 | 3.2 |
| Lung cancer 2 | 68.8 |
| Lung NAT 2 | 8.2 |
| Squamous cell carcinoma 3 | 9.7 |
| Lung NAT 3 | 2.1 |
| metastatic melanoma 1 | 33.4 |
| Melanoma 2 | 5.3 |
| Melanoma 3 | 11.3 |
| metastatic melanoma 4 | 40.3 |
| metastatic melanoma 5 | 100.0 |
| Bladder cancer 1 | 10.3 |
| Bladder cancer NAT 1 | 0.0 |
| Bladder cancer 2 | 5.5 |
| Bladder cancer NAT 2 | 1.8 |
| Bladder cancer NAT 3 | 1.1 |
| Bladder cancer NAT 4 | 27.4 |
| Prostate adenocarcinoma 1 | 8.9 |
| Prostate adenocarcinoma 2 | 12.3 |
| Prostate adenocarcinoma 3 | 20.9 |
| Prostate adenocarcinoma 4 | 4.6 |
| Prostate cancer NAT 5 | 11.6 |
| Prostate adenocarcinoma 6 | 37.9 |
| Prostate adenocarcinoma 7 | 24.7 |
| Prostate adenocarcinoma 8 | 5.5 |
| Prostate adenocarcinoma 9 | 17.8 |
| Prostate cancer NAT 10 | 29.7 |
| Kidney cancer 1 | 8.7 |
| Kidney NAT 1 | 6.3 |
| Kidney cancer 2 | 82.4 |
| Kidney NAT 2 | 18.4 |
| Kidney cancer 3 | 7.3 |
| Kidney NAT 3 | 7.1 |
| Kidney cancer 4 | 8.5 |
| Kidney NAT 4 | 5.5 |

Column A - Rel. Exp. (%) Ag4020 Run 259744763

CNS_neurodegeneration_v1.0 Summary: Ag4020 The CG95430-01 gene was not differentially expressed in the Alzheimer's disease samples represented on this panel. However, this gene was expressed in the brain, with highest expression in the hippocampus of an Alzheimer's patient (CT=31.4). Therapeutic modulation of the activity of this gene or its protein product is useful in the treatment of neurological disorders, such as Alzheimer's disease, Parkinson's disease, schizophrenia, multiple sclerosis, stroke and epilepsy.
Oncology_cell_line_screening_panel_v3.1 Summary: Ag4020 Highest expression of the CG95430-01 gene was detected in an epidermoid carcinoma cell line (CT=32.6). Low expression of this gene was also seen in cell lines derived from fibrosarcoma, pancreatic ductal adenocarcinoma, pancreatic, colon and gastric cancers. Therapeutic modulation of the activity of this gene or its protein product is useful in the treatment of these cancers.
Panel 4.1D Summary: Ag4020 This gene was most highly expressed in a normal kidney sample (CT=32.3). Low but significant levels of expression were also seen in untreated and cytokine activated lung fibroblasts and thymus. These results suggest that this gene is involved in the homeostasis of the lung, thymus, and kidney. Down-regulated expression of this gene in cytokine-activated lung fibroblasts indicates that modulation of this gene and its protein product will help to maintain or restore function to the lung during inflammation.
Panel 5 Islet Summary: Ag4020 The CG95430-01 gene was expressed in adipose and skeletal muscle (CTs=31-34). This gene encodes a putative adiponectin [also known as adipocyte complement-related protein (ACRP-30), AdipoQ, apM1 (adipose most abundant transcript 1) or GBP28 (28 kDa gelatin binding protein)], a member of the Clq family. This protein is induced over 100-fold in adipocyte differentiation (Scherer et al., J Biol Chem Nov. 10, 1995;270(45):26746-9) and is involved in adipocyte signaling (Hu et al., J Biol Chem May 3, 1996;271(18):10697-703). Like other members of the Clq family, it forms a homotrimer and the crystal structure indicates that it likely arose from tumor necrosis factor (TNF; Shapiro and Scherer, Curr Biol Mar. 12, 1998;8(6):335-8). Ionomycin increases expression of adiponectin and dibutyryl cAMP and TNF-alpha reduce expression and secretion in 3T3-L1 adipocytes (Kappes and Loffler, Horm Metab Res 2000 November-December;32(11-12):548-54). Levels of adiponectin are decreased in obese humans (Arita et al., Biochem Biophys Res Commun 1999 April 2;257(1):79-83) and mice (Hu et al., J Biol Chem May 3, 1996;271(18):10697-703). A proteolytic cleavage product of adiponectin is reported to increase fatty acid oxidation in muscle and causes weight loss in mice. (Fruebis et al., Proc Natl Acad Sci USA Feb. 13, 2001;98(4):2005-10). A missense mutation in the protein was correlated with a markedly low plasma adiponectin level (Takahashi et al., Int J Obes Relat Metab Disord 2000 July;24(7):861-8). Recent papers have shown that adiponectin reverses insulin resistance in mouse models of lipoatrophy and obesity (Yamauchi et al., Nature Med 2000; 7(8): 941-6), and that it enhances insulin action on the liver (Berg et al., ibid, 947-53). In addition, circulating levels of adiponectin have been shown to be lower in obese than in lean subjects and lower in diabetic patients than in non-diabetic patients, with particularly low levels in subjects with coronary artery disease. Furthermore, in patients who were subjected to a weight loss program that resulted in a 10% reduction of their body mass index, circulating adiponectin levels increased significantly. (Berg AH. Trends Endocrinol Metab. 2002 March; 13(2):84-9). Based on the homology of CG95430-01 to adiponectin and its expression profile, therapeutic modulation of the activity of this gene or its protein product using nucleic acid, protein, antibody or small molecule drugs is useful for the treatment of obesity, type II diabetes and/or their secondary complications.

Adiponectin also seems to have additional cardiovascular and immune system effects. Levels of this protein are reduced in a cohort of Japanese patients with coronary artery disease (CAD), which correlates with the modulation of endothelial adhesion molecules on treatment of human aortic endothelial cells with adiponectin (Ouchi et al., Circulation Dec. 21-28, 1991; 100(25):2473-6). This protein is found adhering to vascular walls after injury (Okamoto et al. Horm Metab Res 2000 February; 32(2):47-50) and presence of adiponectin suppresses the macrophage to foam cell transformation (Ouchi et al., Circulation Feb. 27, 2001;103(8):1057-63). In addition, levels of adiponectin are lower in diabetic subjects with CAD relative to non-diabetic subjects or diabetic subjects without CAD (Hotta et al., Arterioscler Thromb Vasc Biol 2000 June; 20(6): 1595-9), indicating that lower levels of adiponectin may be an indicator of macroangiopathy in diabetes. Moreover, this protein negatively regulates the growth of myelomonocytic precursors (in part by inducing apoptosis) and macrophage function (Yokota et al., Blood Sep. 1, 2000;96(5):1723-32), potentially via the complement 1Q receptor ClqRp.

The Clq family of proteins includes the complement subunit Clq, gliacolin, Clq-related protein, cerebellin, CORS26 etc., all of which are secreted proteins. These proteins share a common domain, the Clq domain, at the C terminus and collagen triple helix repeats at the C terminus. The repeats enable the proteins to form homotrimers and possibly oligomers. Members of this family have been implicated in tissue differentiation, immune regulation, energy homeostasis, synaptic function and in diseases such as obesity and neurodegeneration. Therapeutic modulation of the activity of this gene or its protein product using nucleic acid, protein, antibody or small molecule drugs is useful in the prevention and/or treatment of obesity and diabetes. Furthermore, development of human monoclonal antibodies that inhibit this Adipo-Q like protein is useful in the therapeutic treatment of cachexia that occurs in many forms of cancer.

General oncology screening panel_v_{—}2.4 Summary: Ag4020 This gene was most highly expressed in a metastatic melanoma (CT=32.7). Significant levels of expression were also seen in a lung cancer and a kidney cancer when compared to normal adjacent tissue. Gene or protein expression levels are useful as a diagnostic marker to detect the presence of these cancers. Therapeutic modulation of the activity of this gene or its protein product is useful in the treatment of kidney cancer, lung cancer, and melanoma.

O.CG95430-02 and CG95430-04

Expression of genes CG95430-02 and CG95430-04 was assessed using the primer-probe set Ag7140, described in Table OA. Results of the RTQ-PCR runs are shown in Table OB. CG95430-02 and CG95430-04 represent the physical clones for mature and full-length gene respectively.

TABLE OA
Probe Name Ag7140

| | | | Start | SEQ ID |
|---|---|---|---|---|
| Primers | Sequences | Length | Position | No |
| Forward | 5′-tttctccaggaatgttca | 21 | 720 | 334 |
| | ggt-3′ | | | |
| Probe | TET-5′-actgcacaccaaag | 26 | 768 | 335 |
| | atgcttacatga-3′-TAMRA | | | |
| Reverse | 5′-cagaggcctggtcc | 17 | 798 | 336 |
| | tca-3′ | | | |

TABLE OB
General screening panel v1.7

| Tissue Name | A |
|---|---|
| Adipose | 100.0 |
| HUVEC | 0.0 |
| Melanoma* Hs688(A).T | 0.0 |
| Melanoma* Hs688(B).T | 0.0 |
| Melanoma (met) SK-MEL-5 | 0.0 |
| Testis | 0.0 |
| Prostate ca. (bone met) PC-3 | 0.0 |
| Prostate ca. DU145 | 0.0 |
| Prostate pool | 0.1 |
| Uterus pool | 1.3 |
| Ovarian ca. OVCAR-3 | 0.0 |
| Ovarian ca. (ascites) SK-OV-3 | 0.0 |
| Ovarian ca. OVCAR-4 | 0.0 |
| Ovarian ca. OVCAR-5 | 0.9 |
| Ovarian ca. IGROV-1 | 0.8 |
| Ovarian ca. OVCAR-8 | 0.0 |
| Ovary | 24.3 |
| Breast ca. MCF-7 | 0.0 |
| Breast ca. MDA-MB-231 | 0.0 |
| Breast ca. BT-549 | 0.0 |
| Breast ca. T47D | 0.0 |
| Breast pool | 0.1 |
| Trachea | 2.4 |
| Lung | 1.7 |
| Fetal Lung | 2.0 |
| Lung ca. NCI-N417 | 1.1 |
| Lung ca. LX-1 | 0.0 |
| Lung ca. NCI-H146 | 1.3 |
| Lung ca. SHP-77 | 0.0 |
| Lung ca. NCI-H23 | 0.0 |
| Lung ca. NCI-H460 | 0.0 |
| Lung ca. HOP-62 | 0.0 |
| Lung ca. NCI-H522 | 1.4 |
| Lung ca. DMS-114 | 0.0 |
| Liver | 0.1 |
| Fetal Liver | 0.0 |
| Kidney pool | 4.5 |
| Fetal Kidney | 2.7 |
| Renal ca. 786-0 | 0.0 |
| Renal ca. A498 | 0.0 |
| Renal ca. ACHN | 0.0 |
| Renal ca. UO-31 | 0.0 |
| Renal ca. TK-10 | 0.0 |
| Bladder | 15.5 |
| Gastric ca. (liver met.) NCI-N87 | 0.0 |
| Stomach | 0.8 |
| Colon ca. SW-948 | 0.2 |
| Colon ca. SW480 | 0.0 |
| Colon ca. (SW480 met) SW620 | 0.0 |
| Colon ca. HT29 | 0.0 |
| Colon ca. HCT-116 | 0.0 |
| Colon cancer tissue | 0.4 |
| Colon ca. SW1116 | 0.5 |
| Colon ca. Colo-205 | 0.0 |
| Colon ca. SW-48 | 0.0 |
| Colon | 1.8 |
| Small Intestine | 2.0 |
| Fetal Heart | 12.3 |
| Heart | 13.4 |
| Lymph Node pool 1 | 0.3 |
| Lymph Node pool 2 | 21.5 |
| Fetal Skeletal Muscle | 30.6 |
| Skeletal Muscle pool | 9.6 |
| Skeletal Muscle | 49.3 |
| Spleen | 1.5 |
| Thymus | 0.4 |
| CNS cancer (glio/astro) SF-268 | 0.0 |
| CNS cancer (glio/astro) T98G | 0.0 |
| CNS cancer (neuro; met) SK-N-AS | 0.0 |
| CNS cancer (astro) SF-539 | 0.0 |
| CNS cancer (astro) SNB-75 | 0.0 |
| CNS cancer (glio) SNB-19 | 0.0 |
| CNS cancer (glio) SF-295 | 0.0 |
| Brain (Amygdala) | 0.4 |
| Brain (Cerebellum) | 0.0 |
| Brain (Fetal) | 1.8 |
| Brain (Hippocampus) | 1.6 |
| Cerebral Cortex pool | 0.5 |
| Brain (Substantia nigra) | 0.2 |
| Brain (Thalamus) | 0.8 |
| Brain (Whole) | 0.7 |
| Spinal Cord | 0.2 |
| Adrenal Gland | 1.0 |
| Pituitary Gland | 0.0 |
| Salivary Gland | 0.8 |
| Thyroid | 2.5 |
| Pancreatic ca. PANC-1 | 0.6 |
| Pancreas pool | 0.0 |

Column A - Rel. Exp. (%) Ag7140, Run 318037557

General_screening_panel_v1.7 Summary: Ag7140 Highest expression of the CG95430-02 and CG95430-04 gene variants was detected in adipose tissue (CT=28.9). Moderate to low expression of these variants was also seen in number of tissues that contribute to metabolism including thyroid, skeletal muscle, heart, small intestine, and colon. Therapeutic modulation of the activity of these gene variants or their protein products using nucleic acid, protein, antibody or small molecule drugs is useful in the treatment of endocrine/metabolically related diseases, such as obesity and diabetes.

P. CG97111-01: Interleukin-1 Receptor Antagonist Protein Precursor

Expression of gene CG97111-01 was assessed using the primer-probe sets Ag4106 described in Tables PA. Results of the RTQ-PCR runs are shown in Tables PB and PC.

TABLE PA
Probe Name Ag4106

| | | | Start | SEQ ID |
|---|---|---|---|---|
| Primers | Sequences | Length | Position | No |
| Forward | 5′-cctctatagtctccggaa | 22 | 897 | 337 |
| | ggaa-3′ | | | |
| Probe | TET-5′-tggatttcagctca | 26 | 935 | 338 |
| | gtgacacccatt-3′-TAMRA | | | |
| Reverse | 5′-gttgtggaggtcagaagt | 22 | 961 | 339 |
| | ctga-3′ | | | |

TABLE PB
General screening panel v1.4

| Tissue Name | A |
|---|---|
| Adipose | 0.0 |
| Melanoma* Hs688(A).T | 0.0 |
| Melanoma* Hs688(B).T | 0.0 |
| Melanoma* M14 | 0.0 |
| Melanoma* LOXIMVI | 0.0 |
| Melanoma* SK-MEL-5 | 0.0 |
| Squamous cell carcinoma SCC-4 | 5.1 |
| Testis Pool | 0.0 |
| Prostate ca.* (bone met) PC-3 | 0.0 |
| Prostate Pool | 0.0 |
| Placenta | 0.0 |
| Uterus Pool | 0.0 |
| Ovarian ca. OVCAR-3 | 12.8 |
| Ovarian ca. SK-OV-3 | 0.0 |
| Ovarian ca. OVCAR-4 | 0.0 |
| Ovarian ca. OVCAR-5 | 0.0 |
| Ovarian ca. IGROV-1 | 0.0 |
| Ovarian ca. OVCAR-8 | 0.0 |
| Ovary | 0.0 |
| Breast ca. MCF-7 | 0.0 |
| Breast ca. MDA-MB-231 | 0.0 |
| Breast ca. BT 549 | 0.0 |
| Breast ca. T47D | 0.0 |
| Breast ca. MDA-N | 0.0 |
| Breast Pool | 0.0 |
| Trachea | 3.1 |
| Lung | 0.0 |
| Fetal Lung | 2.2 |
| Lung ca. NCI-N417 | 0.0 |
| Lung ca. LX-1 | 0.0 |
| Lung ca. NCI-H146 | 0.0 |
| Lung ca. SHP-77 | 0.0 |
| Lung ca. A549 | 0.0 |
| Lung ca. NCI-H526 | 0.0 |
| Lung ca. NCI-H23 | 0.0 |
| Lung ca. NCI-H460 | 0.0 |
| Lung ca. HOP-62 | 0.0 |
| Lung ca. NCI-H522 | 0.0 |
| Liver | 0.0 |
| Fetal Liver | 0.0 |
| Liver ca. HepG2 | 0.0 |
| Kidney Pool | 0.0 |
| Fetal Kidney | 0.0 |
| Renal ca. 786-0 | 0.0 |
| Renal ca. A498 | 0.0 |
| Renal ca. ACHN | 0.0 |
| Renal ca. UO-31 | 0.0 |
| Renal ca. TK-10 | 0.0 |
| Bladder | 0.0 |
| Gastric ca. (liver met.) NCI-N87 | 0.0 |
| Gastric ca. KATO III | 0.0 |
| Colon ca. SW-948 | 0.0 |
| Colon ca. SW480 | 0.0 |
| Colon ca.* (SW480 met) SW620 | 0.0 |
| Colon ca. HT29 | 0.0 |
| Colon ca. HCT-116 | 0.0 |
| Colon ca. CaCo-2 | 0.0 |
| Colon cancer tissue | 9.7 |
| Colon ca. SW1116 | 0.0 |
| Colon ca. Colo-205 | 0.0 |
| Colon ca. SW-48 | 0.0 |
| Colon Pool | 0.0 |
| Small Intestine Pool | 3.3 |
| Stomach Pool | 0.0 |
| Bone Marrow Pool | 0.0 |
| Fetal Heart | 0.0 |
| Heart Pool | 0.0 |
| Lymph Node Pool | 0.0 |
| Fetal Skeletal Muscle | 0.0 |
| Skeletal Muscle Pool | 0.0 |
| Spleen Pool | 0.0 |
| Thymus Pool | 100.0 |
| CNS cancer (glio/astro) U87-MG | 24.8 |
| CNS cancer (glio/astro) U-118-MG | 0.0 |
| CNS cancer (neuro; met) SK-N-AS | 0.0 |
| CNS cancer (astro) SF-539 | 0.0 |
| CNS cancer (astro) SNB-75 | 0.0 |
| CNS cancer (glio) SNB-19 | 0.0 |
| CNS cancer (glio) SF-295 | 0.0 |
| Brain (Amygdala) Pool | 0.0 |
| Brain (cerebellum) | 0.0 |
| Brain (fetal) | 0.0 |
| Brain (Hippocampus) Pool | 0.0 |
| Cerebral Cortex Pool | 0.0 |
| Brain (Substantia nigra) Pool | 5.7 |
| Brain (Thalamus) Pool | 0.0 |
| Brain (whole) | 0.0 |
| Spinal Cord Pool | 0.0 |
| Adrenal Gland | 0.0 |
| Pituitary gland Pool | 0.0 |
| Salivary Gland | 0.0 |
| Thyroid (female) | 0.0 |
| Pancreatic ca. CAPAN2 | 0.0 |
| Pancreas Pool | 0.0 |

Column A - Rel. Exp. (%) Ag4106, Run 219446784

TABLE PC
Panel 4.1D

| Tissue Name | A |
|---|---|
| Secondary Th1 act | 0.0 |
| Secondary Th2 act | 0.0 |
| Secondary Tr1 act | 0.0 |
| Secondary Th1 rest | 0.0 |
| Secondary Th2 rest | 0.0 |
| Secondary Tr1 rest | 0.0 |
| Primary Th1 act | 6.0 |
| Primary Th2 act | 0.0 |
| Primary Tr1 act | 0.0 |
| Primary Th1 rest | 0.0 |
| Primary Th2 rest | 0.0 |
| Primary Tr1 rest | 2.3 |
| CD45RA CD4 lymphocyte act | 0.0 |
| CD45RO CD4 lymphocyte act | 0.0 |
| CD8 lymphocyte act | 0.0 |
| Secondary CD8 lymphocyte rest | 0.0 |
| Secondary CD8 lymphocyte act | 0.0 |
| CD4 lymphocyte none | 0.0 |
| 2ry Th1/Th2/Tr1_anti-CD95 CH11 | 0.0 |
| LAK cells rest | 0.0 |
| LAK cells IL-2 | 0.0 |
| LAK cells IL-2 + IL-12 | 0.0 |
| LAK cells IL-2 + IFN gamma | 0.0 |
| LAK cells IL-2 + IL-18 | 0.0 |
| LAK cells PMA/ionomycin | 10.4 |
| NK Cells IL-2 rest | 0.0 |
| Two Way MLR 3 day | 5.2 |
| Two Way MLR 5 day | 0.0 |
| Two Way MLR 7 day | 0.0 |
| PBMC rest | 0.0 |
| PBMC PWM | 0.0 |
| PBMC PHA-L | 0.0 |
| Ramos (B cell) none | 0.0 |
| Ramos (B cell) ionomycin | 0.0 |
| B lymphocytes PWM | 5.0 |
| B lymphocytes CD40L and IL-4 | 0.0 |
| EOL-1 dbcAMP | 0.0 |
| EOL-1 dbcAMP PMA/ionomycin | 0.0 |
| Dendritic cells none | 0.0 |
| Dendritic cells LPS | 0.0 |
| Dendritic cells anti-CD40 | 0.0 |
| Monocytes rest | 0.0 |
| Monocytes LPS | 0.0 |
| Macrophages rest | 0.0 |
| Macrophages LPS | 0.0 |
| HUVEC none | 0.0 |
| HUVEC starved | 0.0 |
| HUVEC IL-1beta | 0.0 |
| HUVEC IFN gamma | 0.0 |
| HUVEC TNF alpha + IFN gamma | 0.0 |
| HUVEC TNF alpha + IL4 | 0.0 |
| HUVEC IL-11 | 0.0 |
| Lung Microvascular EC none | 0.0 |
| Lung Microvascular EC TNFalpha + IL-1beta | 0.0 |
| Microvascular Dermal EC none | 0.0 |
| Microsvasular Dermal EC TNFalpha + IL-1beta | 0.0 |
| Bronchial epithelium TNFalpha + IL1beta | 7.6 |
| Small airway epithelium none | 11.1 |
| Small airway epithelium TNFalpha + IL-1beta | 0.0 |
| Coronery artery SMC rest | 0.0 |
| Coronery artery SMC TNFalpha + IL-1beta | 0.0 |
| Astrocytes rest | 0.0 |
| Astrocytes TNFalpha + IL-1beta | 2.2 |
| KU-812 (Basophil) rest | 0.0 |
| KU-812 (Basophil) PMA/ionomycin | 0.0 |
| CCD1106 (Keratinocytes) none | 9.5 |
| CCD1106 (Keratinocytes) TNFalpha + IL-1beta | 0.0 |
| Liver cirrhosis | 5.3 |
| NCI-H292 none | 0.0 |
| NCI-H292 IL-4 | 0.0 |
| NCI-H292 IL-9 | 0.0 |
| NCI-H292 IL-13 | 0.0 |
| NCI-H292 IFN gamma | 0.0 |
| HPAEC none | 0.0 |
| HPAEC TNF alpha + IL-1 beta | 0.0 |
| Lung fibroblast none | 0.0 |
| Lung fibroblast TNF alpha + IL-1 beta | 0.0 |
| Lung fibroblast IL-4 | 0.0 |
| Lung fibroblast IL-9 | 0.0 |
| Lung fibroblast IL-13 | 0.0 |
| Lung fibroblast IFN gamma | 0.0 |
| Dermal fibroblast CCD1070 rest | 0.0 |
| Dermal fibroblast CCD1070 TNF alpha | 0.0 |
| Dermal fibroblast CCD1070 IL-1 beta | 0.0 |
| Dermal fibroblast IFN gamma | 0.0 |
| Dermal fibroblast IL-4 | 0.0 |
| Dermal Fibroblasts rest | 0.0 |
| Neutrophils TNFa + LPS | 0.0 |
| Neutrophils rest | 0.0 |
| Colon | 5.4 |
| Lung | 7.6 |
| Thymus | 9.3 |
| Kidney | 100.0 |

Column A - Rel. Exp. (%) Ag4106, Run 172569366

General_screening_panel_v1.4 Summary: Ag4106 Significant expression of the CG97111-01 gene was seen mainly in thymus (CT=33.4). This gene may therefore play an important role in T cell development. Gene or protein expression levels are useful for the detection of thymus. Therapeutic modulation of the activity of this gene or its protein product is useful to modulate immune function (T cell development) and be important for organ transplant, AIDS treatment or post chemotherapy immune reconstitiution.
Panel 4.1D Summary: Ag4106 Significant expression of this gene was seen in a normal kidney sample (CT=33.4). Therapeutic modulation of the activity of this gene or its protein product is useful to modulate kidney function and for the treatment of inflammatory or autoimmune diseases that affect the kidney, including lupus and glomerulonephritis.

### Other Embodiments

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims. The claims presented are representative of the inventions disclosed herein. Other, unclaimed inventions are also contemplated. Applicants reserve the right to pursue such inventions in later claims.

1. An isolated polypeptide comprising the mature form of an amino acid sequenced selected from the group consisting of SEQ ID NO:2n, wherein n is an integer between 1 and 141.

2. An isolated polypeptide comprising an amino acid sequence selected from the group consisting of SEQ ID NO:2n, wherein n is an integer between 1 and 141.

3. An isolated polypeptide comprising an amino acid sequence which is at least 95% identical to an amino acid sequence selected from the group consisting of SEQ ID NO:2n, wherein n is an integer between 1 and 141.

4. An isolated polypeptide, wherein the polypeptide comprises an amino acid sequence comprising one or more conservative substitutions in the amino acid sequence selected from the group consisting of SEQ ID NO:2n, wherein n is an integer between 1 and 141.

5. The polypeptide of claim 1 wherein said polypeptide is naturally occurring.

6. A composition comprising the polypeptide of claim 1 and a carrier.

7. A kit comprising, in one or more containers, the composition of claim 6.

8. The use of a therapeutic in the manufacture of a medicament for treating a syndrome associated with a human disease, the disease selected from a pathology associated with the polypeptide of claim 1, wherein the therapeutic comprises the polypeptide of claim 1.

9. A method for determining the presence or amount of the polypeptide of claim 1 in a sample, the method comprising:
(a) providing said sample;
(b) introducing said sample to an antibody that binds immunospecifically to the polypeptide; and
(c) determining the presence or amount of antibody bound to said polypeptide,
thereby determining the presence or amount of polypeptide in said sample.

10. A method for determining the presence of or predisposition to a disease associated with altered levels of expression of the polypeptide of claim 1 in a first mammalian subject, the method comprising:
a) measuring the level of expression of the polypeptide in a sample from the first mammalian subject; and
b) comparing the expression of said polypeptide in the sample of step (a) to the expression of the polypeptide present in a control sample from a second mammalian subject known not to have, or not to be predisposed to, said disease,
wherein an alteration in the level of expression of the polypeptide in the first subject as compared to the control sample indicates the presence of or predisposition to said disease.

11. A method of identifying an agent that binds to the polypeptide of claim 1, the method comprising:
(a) introducing said polypeptide to said agent; and
(b) determining whether said agent binds to said polypeptide.

12. The method of claim 11 wherein the agent is a cellular receptor or a downstream effector.

13. A method for identifying a potential therapeutic agent for use in treatment of a pathology, wherein the pathology is related to aberrant expression or aberrant physiological interactions of the polypeptide of claim 1, the method comprising:
(a) providing a cell expressing the polypeptide of claim 1 and having a property or function ascribable to the polypeptide;
(b) contacting the cell with a composition comprising a candidate substance; and
(c) determining whether the substance alters the property or function ascribable to the polypeptide;
whereby, if an alteration observed in the presence of the substance is not observed when the cell is contacted with a composition in the absence of the substance, the substance is identified as a potential therapeutic agent.

14. A method for screening for a modulator of activity of or of latency or predisposition to a pathology associated with the polypeptide of claim 1, said method comprising:
(a) administering a test compound to a test animal at increased risk for a pathology associated with the polypeptide of claim 1, wherein said test animal recombinantly expresses the polypeptide of claim 1;
(b) measuring the activity of said polypeptide in said test animal after administering the compound of step (a); and
(c) comparing the activity of said polypeptide in said test animal with the activity of said polypeptide in a control animal not administered said polypeptide, wherein a change in the activity of said polypeptide in said test animal relative to said control animal indicates the test compound is a modulator activity of or latency or predisposition to, a pathology associated with the polypeptide of claim 1.

15. The method of claim 14, wherein said test animal is a recombinant test animal that expresses a test protein transgene or expresses said transgene under the control of a promoter at an increased level relative to a wild-type test animal, and wherein said promoter is not the native gene promoter of said transgene.

16. A method for modulating the activity of the polypeptide of claim 1, the method comprising contacting a cell sample expressing the polypeptide of claim 1 with a compound that binds to said polypeptide in an amount sufficient to modulate the activity of the polypeptide.

17. A method of treating or preventing a pathology associated with the polypeptide of claim 1, the method comprising administering the polypeptide of claim 1 to a subject in which such treatment or prevention is desired in an amount sufficient to treat or prevent the pathology in the subject.

18. The method of claim 17, wherein the subject is a human.

19. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising the amino acid sequence selected from the group consisting of SEQ ID NO:2n, wherein n is an integer between 1 and 141, or a biologically active fragment thereof.

20. An isolated nucleic acid molecule comprising a nucleic acid sequence selected from the group consisting of SEQ ID NO:2n−1, wherein n is an integer between 1 and 141.

21. The nucleic acid molecule of claim 20, wherein the nucleic acid molecule is naturally occurring.

22. A nucleic acid molecule, wherein the nucleic acid molecule differs by a single nucleotide from a nucleic acid sequence selected from the group consisting of SEQ ID NO: 2n−1, wherein n is an integer between 1 and 141.

23. An isolated nucleic acid molecule encoding the mature form of a polypeptide having an amino acid sequence selected from the group consisting of SEQ ID NO:2n, wherein n is an integer between 1 and 141.

24. A composition comprising an isolated nucleic acid molecule, said molecule comprising a nucleic acid sequence selected from the group consisting of SEQ ID NO: 2n−1, wherein n is an integer between 1 and 141, and a carrier.

25. The nucleic acid molecule of claim 20, wherein said nucleic acid molecule hybridizes under stringent conditions to the nucleotide sequence selected from the group consisting of SEQ ID NO: 2n−1, wherein n is an integer between 1 and 141, or a complement of said nucleotide sequence.

26. A vector comprising the nucleic acid molecule of claim 20.

27. The vector of claim 26, further comprising a promoter operably linked to said nucleic acid molecule.

28. A cell comprising the vector of claim 26.

29. An antibody that immunospecifically binds to the polypeptide of claim 1.

30. The antibody of claim 29, wherein the antibody is a monoclonal antibody.

31. The antibody of claim 29, wherein the antibody is a humanized antibody.

32. A method for determining the presence or amount of the nucleic acid molecule of claim 20 in a sample, the method comprising:
(a) providing said sample;
(b) introducing said sample to a probe that binds to said nucleic acid molecule; and
(c) determining the presence or amount of said probe bound to said nucleic acid molecule,
thereby determining the presence or amount of the nucleic acid molecule in said sample.

33. The method of claim 32 wherein presence or amount of the nucleic acid molecule is used as a marker for cell or tissue type.

34. The method of claim 33 wherein the cell or tissue type is cancerous.

35. A method for determining the presence of or predisposition to a disease associated with altered levels of expression of the nucleic acid molecule of claim 20 in a first mammalian subject, the method comprising:
a) measuring the level of expression of the nucleic acid in a sample from the first mammalian subject; and
b) comparing the level of expression of said nucleic acid in the sample of step (a) to the level of expression of the nucleic acid present in a control sample from a second mammalian subject known not to have or not be predisposed to, the disease;
wherein an alteration in the level of expression of the nucleic acid in the first subject as compared to the control sample indicates the presence of or predisposition to the disease.

36. A method of producing the polypeptide of claim 1, the method comprising culturing a cell under conditions that lead to expression of the polypeptide, wherein said cell comprises a vector comprising an isolated nucleic acid molecule comprising a nucleic acid sequence selected from the group consisting of SEQ ID NO:2n−1, wherein n is an integer between 1 and 141.

37. The method of claim 36 wherein the cell is a bacterial cell.

38. The method of claim 36 wherein the cell is an insect cell.

39. The method of claim 36 wherein the cell is a yeast cell.

40. The method of claim 36 wherein the cell is a mammalian cell.

41. A method of producing the polypeptide of claim 2, the method comprising culturing a cell under conditions that lead to expression of the polypeptide, wherein said cell comprises a vector comprising an isolated nucleic acid molecule comprising a nucleic acid sequence selected from the group consisting of SEQ ID NO:2n−1, wherein n is an integer between 1 and 141.

42. The method of claim 41 wherein the cell is a bacterial cell.

43. The method of claim 41 wherein the cell is an insect cell.

44. The method of claim 41 wherein the cell is a yeast cell.

45. The method of claim 41 wherein the cell is a mammalian cell.

46. An isolated polypeptide comprising an amino acid sequence at least 95% similar to SEQ ID NO: 2, wherein said amino acid sequence comprises at least one amino acid substitution, wherein said substitution is at amino acid position 43 when numbered in accordance with SEQ ID NO: 2.

47. An isolated nucleic acid molecule comprising an nucleic acid sequence at least 95% similar to SEQ ID NO: 1, wherein said nucleic acid sequence comprises at least one nucleic acid substitution, wherein said substitution is at nucleic acid position 135 when numbered in accordance with SEQ ID NO: 1.

48. An isolated polypeptide comprising an amino acid sequence at least 95% similar to SEQ ID NO: 14, wherein said amino acid sequence comprises at least one amino acid substitution, wherein said substitution is at the amino acid position selected from the group consisting of 8, 54, 56, 92, 207, 240, 706, 891 and 923 when numbered in accordance with SEQ ID NO: 14.

49. An isolated nucleic acid molecule comprising an nucleic acid sequence at least 95% similar to SEQ ID NO: 13, wherein said nucleic acid sequence comprises at least one nucleic acid substitution, wherein said substitution is at the nucleic acid position selected from the group consisting of 272, 410, 416, 523, 869, 967, 2366, 2921 and 3018 when numbered in accordance with SEQ ID NO: 13.

50. An isolated polypeptide comprising an amino acid sequence at least 95% similar to SEQ ID NO: 58, wherein said amino acid sequence comprises at least one amino acid substitution, wherein said substitution is at the amino acid position selected from the group consisting of 23, 56, 105, 125, 160, 183 and 215 when numbered in accordance with SEQ ID NO: 58.

51. An isolated nucleic acid molecule comprising an nucleic acid sequence at least 95% similar to SEQ ID NO: 57, wherein said nucleic acid sequence comprises at least one nucleic acid substitution, wherein said substitution is at the nucleic acid position selected from the group consisting of 181, 278, 426, 485, 591, 661 and 756 when numbered in accordance with SEQ ID NO: 57.

52. An isolated polypeptide comprising an amino acid sequence at least 95% similar to SEQ ID NO: 80, wherein said amino acid sequence comprises at least one amino acid substitution, wherein said substitution is at amino acid position 219 when numbered in accordance with SEQ ID NO: 80.

53. An isolated nucleic acid molecule comprising an nucleic acid sequence at least 95% similar to SEQ ID NO: 79, wherein said nucleic acid sequence comprises at least one nucleic acid substitution, wherein said substitution is at nucleic acid position 685 when numbered in accordance with SEQ ID NO: 79.

54. An isolated polypeptide comprising an amino acid sequence at least 95% similar to SEQ ID NO: 92, wherein said amino acid sequence comprises at least one amino acid substitution, wherein said substitution is at amino acid position 470 when numbered in accordance with SEQ ID NO: 92.

55. An isolated nucleic acid molecule comprising an nucleic acid sequence at least 95% similar to SEQ ID NO: 91, wherein said nucleic acid sequence comprises at least one nucleic acid substitution, wherein said substitution is at nucleic acid position 1874 when numbered in accordance with SEQ ID NO: 91.

56. An isolated polypeptide comprising an amino acid sequence at least 95% similar to SEQ ID NO: 100, wherein said amino acid sequence comprises at least one amino acid substitution, wherein said substitution is at the amino acid position selected from the group consisting of 11, 112 and 145 when numbered in accordance with SEQ ID NO: 100.

57. An isolated nucleic acid molecule comprising an nucleic acid sequence at least 95% similar to SEQ ID NO: 99, wherein said nucleic acid sequence comprises at least one nucleic acid substitution, wherein said substitution is at the nucleic acid position selected from the group consisting of 80, 383 and 482 when numbered in accordance with SEQ ID NO: 99.

58. An isolated polypeptide comprising an amino acid sequence at least 95% similar to SEQ ID NO: 122, wherein said amino acid sequence comprises at least one amino acid substitution, wherein said substitution is at the amino acid position selected from the group consisting of 12, 38, 54, 65, 66, 69, 80, 90, 91, 96, 100, 101, 102, 114, 122, 125, 126, 134, 135, 144, 148, 154, 155 and 156 when numbered in accordance with SEQ ID NO: 122.

59. An isolated nucleic acid molecule comprising an nucleic acid sequence at least 95% similar to SEQ ID NO: 121, wherein said nucleic acid sequence comprises at least one nucleic acid substitution, wherein said substitution is at the nucleic acid position selected from the group consisting of 35, 112, 160, 194, 197, 206, 240, 269, 273, 287, 298, 301, 305, 340, 365, 374, 376, 400, 404, 431, 442, 461, 463 and 468 when numbered in accordance with SEQ ID NO: 121.
